(19) United States
(12) Patent Application Publication
(10) Pub. No.: US 2023/0022724 A1
(43) Pub. Date: Jan. 26, 2023

(54) NOVEL IMIDAZOPYRAZINE DERIVATIVES

(71) Applicant: Hoffmann-La Roche Inc., Little Falls, NJ (US)

(72) Inventors: Zhanling CHENG, Shanghai (CN); Nawaz KHAN, Cambridge (GB); Christian KRAMER, Loerrach (DE); Christian LERNER, Binningen (CH); Philippe PFLIEGER, Schwoben (FR); Theodor STOLL, Binningen (CH); Jianhua WANG, Shanghai (CN); Yongguang WANG, Shanghai (CN); Song YANG, Shanghai (CN)

(73) Assignee: Hoffmann-La Roche Inc., Little Falls, NJ (US)

(21) Appl. No.: 17/349,760

(22) Filed: Jun. 16, 2021

Related U.S. Application Data
(63) Continuation of application No. PCT/EP2019/085220, filed on Dec. 16, 2019.

(30) Foreign Application Priority Data
Dec. 17, 2018 (CN) PCT/CN2018/121481
Nov. 7, 2019 (CN) PCT/CN2019/116361

(51) Int. Cl. C07D 487/04; A61P 31/04

(52) U.S. Cl. CPC C07D 487/04; A61P 31/04

(57) ABSTRACT

Provided are novel imidazopyrazine derivatives having the general formula (I), wherein A and R¹ to R¹¹ are as described herein

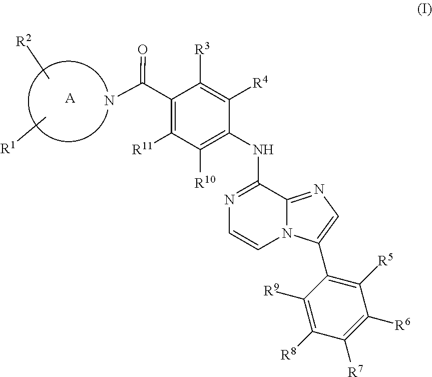

and pharmaceutically acceptable salts thereof.

Further provided are pharmaceutical compositions including the compounds, processes of manufacturing the compounds and methods of using the compounds as medicaments, in particular methods of using the compounds as antibiotics for the treatment or prevention of bacterial infections and resulting diseases.

### CROSS-REFERENCE TO RELATED APPLICATIONS

This application is a Continuation application of International Patent Application No. PCT/EP2019/085220, filed on Dec. 16, 2019, which claims benefit of priority to International Patent Application No. PCT/CN2019/116361, filed on Nov. 7, 2019, and International Patent Application No. PCT/CN2018/121481, filed on Dec. 17, 2018, all of which are incorporated herein by reference in their entirety.

### BACKGROUND

Certain embodiments of the present invention relate to novel imidazopyrazine derivatives which exhibit antibacterial properties. Certain embodiments of the invention also relate to methods of using the compounds for the treatment or prevention of bacterial infections and resulting diseases, in particular for the treatment or prevention of infections with Acinetobacter baumannii and resulting diseases.

Acinetobacter baumannii is a Gram-negative, aerobic, nonfermenting bacterium recognized over the last decades as an emergining pathogen with very limited treatment options.

A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents.

A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistance that allows its survival and spread in the nosocomial setting, making this organism a frequent cause of outbreaks of infection and an endemic, health care-associated pathogen.

Due to increasing antibiotic resistance to most if not all available therapeutic options, Multi-Drug Resistant (MDR) A. baumannii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

The present invention provides novel compounds which exhibit activity against drug-susceptible as well as drug-resistant strains of Acinetobacter baumannii.

### SUMMARY OF THE DISCLOSURE

In a first aspect, the present invention provides compounds of formula (I)

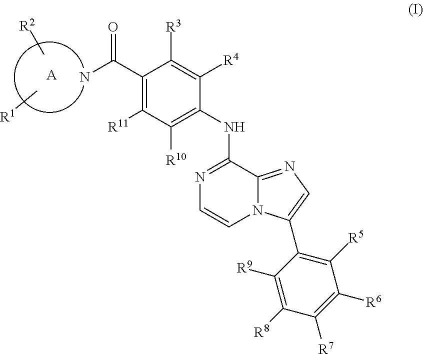

or a pharmaceutically acceptable salt thereof, wherein A and R¹ to R¹¹ are as described herein.

In one aspect, the present invention provides a process of manufacturing the compounds of formula (I) described herein, comprising:
(i) reacting a carboxylic acid IVa, wherein R³ to R¹¹ are as defined herein,

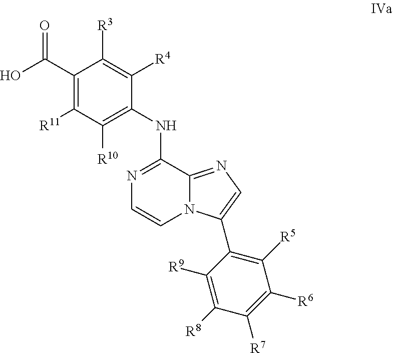

with an amine V, wherein A, R¹ and R² are as defined herein,

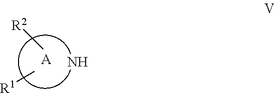

in the presence of a coupling reagent (such as HATU, TBTU, and the like) and a base (such as DIPEA, NEt₃, and the like), to form said compound of formula (I); or
(ii) reacting a compound VI, wherein R¹ to R⁴, R¹⁰, R¹¹ and A are as defined herein and X is halogen,

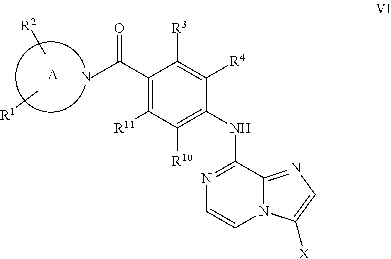

with a boronic acid VII, wherein R⁵ to R⁹ are as defined herein and Y is a boronic acid or a boronic acid ester,

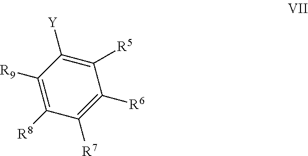

in the presence of a transition metal catalyst (such as PdCl₂(dppf)-CH₂Cl₂ adduct, Pd(PPh₃)₄, and the like) and a base (such as K₃PO₄, NaOtBu, and the like) to form said compound of formula (I).

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a further aspect, the present invention provides a compound of formula (I) as described herein, or a pharmaceutically acceptable salt thereof, for use in the treatment or prevention of infections and resulting diseases caused by Gram-negative bacteria.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

### DETAILED DESCRIPTION OF THE DISCLOSURE

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkenyl” denotes a monovalent linear or branched hydrocarbon group of 2 to 6 carbon atoms with at least one double bond (“C₂-C₆-alkenyl”). In particular embodiments, alkenyl has 2 to 4 carbon atoms with at least one double bond. Examples of alkenyl include ethenyl, propenyl, prop-2-enyl, isopropenyl, n-butenyl and iso-butenyl. Particular alkenyl group is ethenyl.

The term “alkynyl” denotes a monovalent linear or branched hydrocarbon group of 2 to 6 carbon atoms with at least one triple bond (“C₂-C₆-alkynyl”). In particular embodiments, alkynyl has 2 to 4 carbon atoms with at least one triple bond. Examples of alkynyl include ethynyl, propynyl, n-butynyl or isobutynyl. Preferred alkenyl is propynyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “alkynyloxy” refers to an alkynyl group, as previously defined, attached to the parent molecular moiety via an oxygen atom.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 12 ring carbon atoms (“C₃-C₁₂-cycloalkyl”). In some preferred embodiments, the cycloalkyl group is a saturated monocyclic hydrocarbon group of 3 to 10 ring carbon atoms, in particular 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl and cycloheptyl.

The term “cycloalkyloxy” refers to a group cycloalkyl-O—, i.e. a cycloalkyl group substituted with an oxy group and attached to the parent molecular moiety via said oxy group.

The term “cyanocycloalkyloxy” refers to a cycloalkyloxy group, wherein at least one of the hydrogen atoms of the cycloalkyloxy group has been replaced by a cyano group. Preferably, “cyanocycloalkyloxy” refers to a cycloalkyloxy group wherein 1, 2 or 3 hydrogen atoms of the cycloalkyloxy group have been replaced by a cyano group.

The term “aminoalkynyloxy” refers to an alkynyloxy group, wherein at least one of the hydrogen atoms of the alkynyloxy group has been replaced by an amino group. Preferably, “aminoalkynyloxy” refers to an alkynyloxy group wherein 1, 2 or 3 hydrogen atoms of the alkynyloxy group have been replaced by an amino group. A preferred, yet non-limiting example of aminoalkynyloxy is 3-aminoprop-1-ynyl.

The term “aminoalkoxy” refers to an alkoxy group, wherein at least one of the hydrogen atoms of the alkoxy group has been replaced by an amino group. Preferably, “aminoalkoxy” refers to an alkoxy group wherein 1, 2 or 3 hydrogen atoms of the alkoxy group have been replaced by an amino group. A preferred, yet non-limiting example of aminoalkoxy is aminomethoxy.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms of the alkyl group have been replaced by an amino group. A preferred, yet non-limiting example of aminoalkyl is aminomethyl.

The term “carboxyalkyl” refers to an alkyl group, wherein at least one of the hydrogen atoms of the alkyl group has been replaced by a carboxy group. Preferably, “carboxyalkyl” refers to an alkyl group wherein 1, 2 or 3 hydrogen atoms of the alkyl group have been replaced by a carboxy group. A preferred, yet non-limiting example of aminoalkyl is carboxymethyl.

The term “aminoalkoxyalkynyloxy” refers to an alkynyloxy group, wherein at least one of the hydrogen atoms of the alkynyloxy group has been replaced by an aminoalkoxy group. Preferably, “aminoalkoxyalkynyloxy” refers to an alkynyloxy group wherein 1, 2 or 3 hydrogen atoms of the alkynyloxy group have been replaced by an aminoalkoxy group.

The term “hydroxyalkynyloxy” refers to an alkynyloxy group, wherein at least one of the hydrogen atoms of the alkynyloxy group has been replaced by a hydroxy group. Preferably, “hydroxyalkynyloxy” refers to an alkynyloxy group wherein 1, 2 or 3 hydrogen atoms of the alkynyloxy group have been replaced by a hydroxy group. A preferred, yet non-limiting example of hydroxyalkynyloxy is 3-hydroxyprop-1-ynyl.

The terms “heterocycloalkyl” and “heterocyclyl” are used interchangeably and refer to a saturated or partly unsaturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of monocyclic heterocyclyl groups include azetidin-3-yl, azetidin-2-yl, oxetan-3-yl, oxetan-2-yl, 2-oxopyrrolidin-1-yl, 2-oxopyrrolidin-3-yl, 5-oxopyrrolidin-2-yl, 5-oxopyrrolidin-3-yl, 2-oxo-1-piperidyl, 2-oxo-3-piperidyl, 2-oxo-4-piperidyl, 6-oxo-2-piperidyl, 6-oxo-3-piperidyl, 1-piperidinyl, 2-piperidinyl, 3-piperidinyl, 4-piperidinyl, morpholino, morpholin-2-yl and morpholin-3-yl.

The term “heterocyclylalkyl” refers to an alkyl group, wherein at least one of the hydrogen atoms of the alkyl group has been replaced by a heterocyclyl group. Preferably, “heterocyclylalkyl” refers to an alkyl group wherein 1, 2 or 3 hydrogen atoms, most preferably 1 hydrogen atom of the alkyl group have been replaced by a heterocyclyl group.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl and oxazol-5-yl. A particularly preferred, yet non-limiting example of heteroaryl is indolyl, in particular 1H-indol-3-yl.

The term “alkylheteroaryl” refers to a heteroaryl group, wherein at least one of the hydrogen atoms of the heteroaryl group has been replaced by an alkyl group. Preferably, “alkylheteroaryl” refers to a heteroaryl group wherein 1, 2 or 3 hydrogen atoms, most preferably 1 hydrogen atom of the heteroaryl group have been replaced by an alkyl group.

The term “heteroaryloxy” refers to a heteroaryl group attached to the parent molecular moiety via an oxygen atom.

The term “hydroxy” refers to an —OH group.

The term “amino” refers to an —NH₂ group.

The term “cyano” refers to a —CN (nitrile) group.

The term “sulfamoyl” refers to a —SO₂—NH₂ group.

The term “alkylsulfamoyl” refers to a —SO₂—NH(alkyl) group.

The term “dialkylsulfamoyl” refers to a —SO₂—N(alkyl)₂ group.

The term “alkylsulfonyl” refers to a —SO₂-alkyl group.

The term “alkylsulfonyloxy” refers to a —O—SO₂-alkyl group.

The term “alkylsulfanyl” refers to a —S-alkyl group.

The term “carboxy” refers to a —COOH group.

The term “carbamimidoyl” refers to a

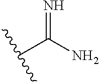 [SMILES: *C(=N)N]

group.

The term “guanidino” refers to a

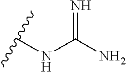 [SMILES: *NC(=N)N]

group.

The term “ureido” refers to a

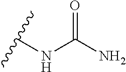 [SMILES: *NC(N)=O]

group.

The term “carbamoyl” refers to a —C(O)NH₂ group.

The term “carbonyl” refers to a —C(O)— group.

The term “alkoxycarbonyl” refers to a —C(O)—O-alkyl group (i.e., an alkyl ester).

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl and trifluoroethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “cyanoalkoxy” refers to an alkoxy group, wherein at least one of the hydrogen atoms of the alkoxy group has been replaced by a cyano group. Preferably, “cyanoalkoxy” refers to an alkoxy group wherein 1, 2 or 3 hydrogen atoms of the alkoxy group have been replaced by a cyano group. A particularly preferred, yet non-limiting example of cyanoalkoxy is cyanomethoxy.

The term “cyanoalkyl” refers to an alkyl group, wherein at least one of the hydrogen atoms of the alkyl group has been replaced by a cyano group. Preferably, “cyanoalkyl” refers to an alkyl group wherein 1, 2 or 3 hydrogen atoms of the alkyl group have been replaced by a cyano group. A particularly preferred, yet non-limiting example of cyanoalkyl is cyanomethyl.

The term “alkoxyalkynyloxy” refers to an alkynyloxy group, wherein at least one of the hydrogen atoms of the alkynyloxy group has been replaced by an alkoxy group. Preferably, “alkoxyalkynyloxy” refers to an alkynyloxy group wherein 1, 2 or 3 hydrogen atoms of the alkynyloxy group have been replaced by an alkoxy group.

The term “cycloalkylalkoxy” refers to an alkoxy group, wherein at least one of the hydrogen atoms of the alkoxy group has been replaced by a cycloalkyl group. Preferably, “cycloalkylalkoxy” refers to an alkoxy group wherein 1, 2 or 3 hydrogen atoms, most preferably 1 hydrogen atom of the alkoxy group have been replaced by a cycloalkyl group. A particularly preferred, yet non-limiting example of cycloalkylalkoxy is cyclopropylmethoxy.

The term “cyanocycloalkylalkoxy” refers to a cycloalkylalkoxy group, wherein at least one of the hydrogen atoms of the cycloalkylalkoxy group has been replaced by a cyano group. Preferably, “cyanocycloalkylalkoxy” refers to a cycloalkylalkoxy group wherein 1, 2 or 3 hydrogen atoms, most preferably 1 hydrogen atom of the cycloalkylalkoxy group have been replaced by a cyano group.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl and hydroxyethyl (e.g. 2-hydroxyethyl). A particularly preferred, yet non-limiting example of hydroxyalkyl is hydroxymethyl.

The term “hydroxyalkoxy” refers to an alkoxy group, wherein at least one of the hydrogen atoms of the alkoxy group has been replaced by a hydroxy group. Preferably, “hydroxyalkoxy” refers to an alkoxy group wherein 1, 2 or 3 hydrogen atoms, most preferably 1 hydrogen atom of the alkoxy group have been replaced by a hydroxy group. Preferred, yet non-limiting examples of hydroxyalkoxy are hydroxymethoxy and hydroxyethoxy (e.g. 2-hydroxyethoxy). A particularly preferred, yet non-limiting example of hydroxyalkoxy is hydroxymethoxy.

The term “hydroxyalkoxyalkyl” refers to an alkyl group, wherein at least one of the hydrogen atoms of the alkyl group has been replaced by a hydroxyalkoxy group. Preferably, “hydroxyalkoxyalkyl” refers to an alkyl group wherein 1, 2 or 3 hydrogen atoms, most preferably 1 hydrogen atom of the alkyl group have been replaced by a hydroxyalkoxy group. A preferred, yet non-limiting example of hydroxyalkoxyalkyl is 2-hydroxyethoxymethyl.

The term “alkoxycarbonylalkoxy” refers to an alkoxy group, wherein at least one of the hydrogen atoms of the alkoxy group has been replaced by an alkoxycarbonyl group. Preferably, “alkoxycarbonylalkoxy” refers to an alkoxy group wherein 1, 2 or 3 hydrogen atoms, most preferably 1 hydrogen atom of the alkoxy group have been replaced by an alkoxycarbonyl group. A preferred, yet non-limiting example of alkoxycarbonylalkoxy is 2-methoxy-2-oxo-ethoxy.

The term “arylalkoxy” refers to an alkoxy group, wherein at least one of the hydrogen atoms of the alkoxy group has been replaced by an aryl group. Preferably, “arylalkoxy” refers to an alkoxy group wherein 1, 2 or 3 hydrogen atoms, most preferably 1 hydrogen atom of the alkoxy group have been replaced by an aryl group. A particularly preferred, yet non-limiting example of arylalkoxy is benzyloxy.

The term “heteroarylalkoxy” refers to an alkoxy group, wherein at least one of the hydrogen atoms of the alkoxy group has been replaced by a heteroaryl group. Preferably, “heteroarylalkoxy” refers to an alkoxy group wherein 1, 2 or 3 hydrogen atoms, most preferably 1 hydrogen atom of the alkoxy group have been replaced by a heteroaryl group.

The term “alkoxyalkyl” refers to an alkyl group, wherein at least one of the hydrogen atoms of the alkyl group has been replaced by an alkoxy group. Preferably, “alkoxyalkyl” refers to an alkyl group wherein 1, 2 or 3 hydrogen atoms, most preferably 1 hydrogen atom of the alkyl group have been replaced by an alkoxy group. A particularly preferred, yet non-limiting example of alkoxyalkyl is 2-methoxyethyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “mammal” as used herein includes both humans and non-humans and includes but is not limited to humans, non-human primates, canines, felines, murines, bovines, equines, and porcines. In a particularly preferred embodiment, the term “mammal” refers to humans.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

### Compounds of the Invention

In a first aspect, the present invention provides compounds of formula (I)

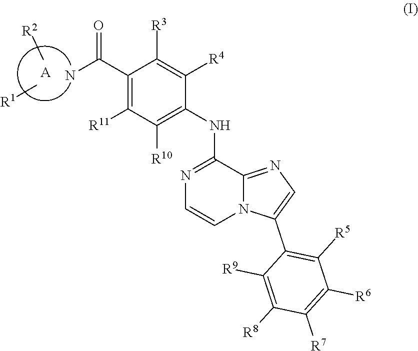

or a pharmaceutically acceptable salt thereof, wherein:
A is a mono- or bicyclic C₂-C₉-heterocycloalkyl ring;
R¹ is hydrogen, amino, C₁-C₆-alkyl-NH—, (C₁-C₆-alkyl)₂N—, sulfamoyl, C₁-C₆-alkylsulfamoyl, di-C₁-C₆-alkylsulfamoyl, C₁-C₆-alkylsulfonyl-NH—C(O)—, C₁-C₆-alkylsulfonyl-N(C₁-C₆-alkyl)-C(O)—, hydroxy, carboxy, carbamimidoyl, carbamoyl, C₁-C₆-alkoxycarbonyl, C₁-C₆-alkoxycarbonyl-NH—, C₁-C₆-alkoxycarbonyl-N(C₁-C₆-alkyl)-, carboxy-NH—, carboxy-N(C₁-C₆-alkyl)-, a group

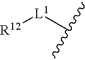

or a group

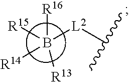

R² is hydrogen, hydroxy, carbamoyl, C₁-C₆-alkyl-NH—C(O)— or (C₁-C₆-alkyl)₂N—C(O)—;
R³ is hydrogen, halogen, NO₂ or CN;
R⁵, R⁶, R⁷, R⁸ and R⁹ are each independently hydrogen, halogen, C₁-C₆-alkoxycarbonyl-C₁-C₆-alkoxy, amino, C₁-C₆-alkyl-NH—, (C₁-C₆-alkyl)₂N—, hydroxy, C₁-C₆-alkoxy, C₁-C₆-alkylsulfanyl, C₁-C₆-alkylsulfonyloxy, C₃-C₁₂-cycloalkyl-C₁-C₆-alkoxy, halo-C₁-C₆-alkoxy, C₆-C₁₄-aryl-C₁-C₆-alkoxy, C₁-C₁₃-heteroaryloxy, C₁-C₁₃-heteroaryl-C₁-C₆-alkoxy, cyano-C₁-C₆-alkoxy, C₃-C₁₂-cycloalkyloxy, C₂-C₆-alkynyloxy, C₁-C₆-alkoxy-C₂-C₆-alkynyloxy, cyano-C₃-C₁₂-cycloalkyloxy, cyano-C₃-C₁₂-cycloalkyl-C₁-C₆-alkoxy, amino-C₂-C₆-alkynyloxy, hydroxy-C₂-C₆-alkynyloxy, halo-C₁-C₆-alkyl, sulfamoyl, C₁-C₆-alkylsulfamoyl, C₁-C₆-alkyl, amino-C₁-C₆-alkoxy-C₂-C₆-alkynyloxy or amino-C₁-C₆-alkoxy;
R⁴, R¹⁰ and R¹¹ are each independently hydrogen, halogen or C₁-C₆-alkyl;
R¹² is C₁-C₆-alkyl substituted with R¹⁷, R¹⁸, R¹⁹, R²⁰ or R²¹, or a combination thereof;
R¹³, R¹⁴, R¹⁵ and R¹⁶ are each independently hydrogen, halogen, cyano, hydroxy, C₁-C₆-alkylsulfonyl, amino, HO—SO₂—, C₁-C₆-alkyl-NH—, (C₁-C₆-alkyl)₂N—, C₁-C₆-alkyl, C₁-C₆-alkoxy, amino-C₁-C₆-alkyl, C₁-C₆-alkyl-NH—C₁-C₆-alkyl-, (C₁-C₆-alkyl)₂N—C₁-C₆-alkyl-, hydroxy-C₁-C₆-alkyl, C₃-C₁₂-cycloalkyl, C₁-C₁₃-heteroaryl, C₁-C₆-alkyl-C₁-C₁₃-heteroaryl, C₂-C₉-heterocycloalkyl-C₁-C₆-alkyl-, carbamoyl, C₁-C₆-alkyl-NH—C(O)—, (C₁-C₆-alkyl)₂N—C(O)— or carboxy;
R¹⁷, R¹⁸, R¹⁹, R²⁰ and R²¹ are each independently hydrogen, HO—SO₂—, hydroxy, cyano, amino, C₁-C₆-alkyl-NH—, (C₁-C₆-alkyl)₂N—, cyano-C₁-C₆-alkyl-NH—, cyano-C₁-C₆-alkyl-N(C₁-C₆-alkyl)-, amino-C₁-C₆-alkyl-C(O)—NH—, C₁-C₆-alkyl-NH—C₁-C₆-alkyl-C(O)—NH—, (C₁-C₆-alkyl)₂N—C₁-C₆-alkyl-C(O)—NH—, amino-C₁-C₆-alkyl-C(O)—N(C₁-C₆-alkyl)-, C₁-C₆-alkyl-NH—C₁-C₆-alkyl-C(O)—N(C₁-C₆-alkyl)-, (C₁-C₆-alkyl)₂N—C₁-C₆-alkyl-C(O)—N(C₁-C₆-alkyl)-, hydroxy-C₁-C₆-alkyl-NH—, hydroxy-C₁-C₆-alkyl-C(O)—NH—, hydroxy-C₁-C₆-alkyl-C(O)—N(C₁-C₆-alkyl)-, guanidino, carboxy, C₁-C₆-alkoxycarbonyl, C₁-C₆-alkoxycarbonyl-NH—, carbamoyl, C₁-C₆-alkyl-NH—C(O)—, (C₁-C₆-alkyl)₂N—C(O)—, C₁-C₆-alkyl-C(O)—NH—, C₁-C₆-alkyl-C(O)—N(C₁-C₆-alkyl)-, hydroxy-C₁-C₆-alkoxy, C₁-C₆-alkoxy, amino-C₁-C₆-alkyl-CH(NH₂)—C(O)—NH—, carboxy-C₁-C₆-alkyl-CH(NH₂)—C(O)—NH—, carboxy-CH(NH₂)—C₁-C₆-alkyl-C(O)—NH—, amino-C₁-C₆-alkyl-CH(COOH)—NH—, carboxy-C₁-C₆-alkyl-N(C₁-C₆-alkyl)-, carboxy-C₁-C₆-alkyl-NH—, ureido, amino-C₁-C₆-alkyl, C₁-C₆-alkyl-NH—C₁-C₆-alkyl- or (C₁-C₆-alkyl)₂N—C₁-C₆-alkyl-;
L¹ is a covalent bond, carbonyl, —NH—, —N(C₁-C₆-alkyl)-, —NH—C(O)—, —C(O)—NH—, —C(O)—N(C₁-C₆-alkyl)- or —N(C₁-C₆-alkyl)-C(O)—;
L² is a covalent bond, —C₁-C₆-alkyl-, carbonyl, SO₂, —C(O)—C₁-C₆-alkyl-, —C₁-C₆-alkyl-C(O)—, —C₁-C₆-alkyl-NH—C(O)—, —C₁-C₆-alkyl-N(C₁-C₆-alkyl)-C(O)—, —C₁-C₆-alkyl-O—C(O)—, —NH—C(O)—, —CH(NH₂)—C(O)—, —O—, —NH—C₁-C₆-alkyl-, —N(C₁-C₆-alkyl)-C₁-C₆-alkyl-, —C(O)—NH—C₁-C₆-alkyl-, —C(O)—N(C₁-C₆-alkyl)-C₁-C₆-alkyl-, —C₁-C₆-alkyl-CH(NH₂)—C(O)—, or —C(O)—NH—; and
B is C₆-C₁₄-aryl, C₁-C₁₃-heteroaryl, C₃-C₁₂-cycloalkyl, or C₂-C₉-heterocycloalkyl.

In one embodiment, the present invention provides a compound of formula (I) as described herein, or a pharmaceutically acceptable salt thereof, wherein R¹ is hydrogen, (C₁-C₆-alkyl)₂N—, hydroxy, carboxy, a group

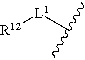

or a group

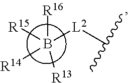

wherein R¹², R¹³, R¹⁴, R¹⁵, R¹⁶, L¹, L² and B are as defined herein.

In a preferred embodiment, the present invention provides a compound of formula (I) as described herein, or a pharmaceutically acceptable salt thereof, wherein R¹ is a group

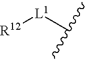

or a group

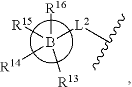

wherein R¹², R¹³, R¹⁴, R¹⁵, R¹⁶, L¹, L² and B are as defined herein.

In a particularly preferred embodiment, the present invention provides a compound of formula (I) as described herein, or a pharmaceutically acceptable salt thereof, wherein R¹ is 2-(dimethylamino)ethyl, 2-(methylamino)ethylcarbamoyl, 2-(methylamino)acetyl, 3-(hydroxymethyl)piperazine-1-carbonyl, 2-(methylamino)ethyl, azetidin-3-ylmethylcarbamoyl or (2S,4R)-4-hydroxypyrrolidine-2-carbonyl.

In one embodiment, the present invention provides a compound of formula (I) as described herein or a pharmaceutically acceptable salt thereof, wherein R¹ is hydrogen, (C₁-C₆-alkyl)₂N—, hydroxy, carboxy, a group

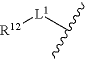

or a group

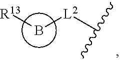

wherein:
R¹² is C₁-C₆-alkyl substituted with R¹⁷, R¹⁸, R¹⁹, R²⁰ or R²¹, or a combination thereof;
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl, C₁-C₆-alkylsulfonyl, C₁-C₁₃-heteroaryl or C₂-C₉-heterocycloalkyl-C₁-C₆-alkyl-;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen, hydroxy or amino;
R¹⁹, R²⁰ and R²¹ are each independently hydrogen or hydroxy;
L¹ is a covalent bond, carbonyl, —N(C₁-C₆-alkyl)-, —NH—C(O)— or —N(C₁-C₆-alkyl)-C(O)—;
L² is a covalent bond, —C₁-C₆-alkyl-, carbonyl, —C₁-C₆-alkyl-C(O)—, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-; and
B is C₁-C₁₃-heteroaryl or C₂-C₉-heterocycloalkyl.

In a preferred embodiment, the present invention provides a compound of formula (I) as described herein or a pharmaceutically acceptable salt thereof, wherein R¹ is a group

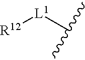

or a group

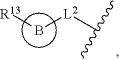

wherein:
R¹² is C₁-C₆-alkyl substituted with R¹⁷;
R¹³ is hydrogen or hydroxy;
R¹⁷ is C₁-C₆-alkyl-NH— or (C₁-C₆-alkyl)₂N—;
L¹ is a covalent bond, carbonyl or —NH—C(O)—;
L² is carbonyl or —C₁-C₆-alkyl-NH—C(O)—; and
B is C₂-C₉-heterocycloalkyl.

In a particularly preferred embodiment, the present invention provides a compound of formula (I) as described herein or a pharmaceutically acceptable salt thereof, wherein R¹ is a group

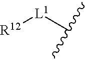

or a group

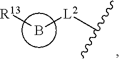

wherein:
R¹² is C₁-C₂-alkyl substituted with R¹⁷;
R¹³ is hydrogen or hydroxy;
R¹⁷ is methylamino or dimethylamino;
L¹ is a covalent bond, carbonyl or —NH—C(O)—;
L² is carbonyl or —CH₂NH—C(O)—; and
B is piperazinyl, pyrrolidinyl or azetidinyl.

In one embodiment, the present invention provides a compound of formula (I) as described herein, or a pharmaceutically acceptable salt thereof, wherein R² is hydrogen or hydroxy.

In one embodiment, the present invention provides a compound of formula (I) as described herein, or a pharmaceutically acceptable salt thereof, wherein R³ is hydrogen, halogen or CN.

In a preferred embodiment, the present invention provides a compound of formula (I) as described herein, or a pharmaceutically acceptable salt thereof, wherein R³ is halogen.

In a particularly preferred embodiment, the present invention provides a compound of formula (I) as described herein, or a pharmaceutically acceptable salt thereof, wherein R³ is chloro.

In a preferred embodiment, the present invention provides a compound of formula (I) as described herein, or a pharmaceutically acceptable salt thereof, wherein R⁴ is hydrogen.

In a preferred embodiment, the present invention provides a compound of formula (I) as described herein, or a pharmaceutically acceptable salt thereof, wherein R⁵ is hydrogen or halogen.

In a particularly preferred embodiment, the present invention provides a compound of formula (I) as described herein, or a pharmaceutically acceptable salt thereof, wherein R⁵ is hydrogen, fluoro or chloro.

In a preferred embodiment, the present invention provides a compound of formula (I) as described herein, or a pharmaceutically acceptable salt thereof, wherein R⁶ is hydrogen or halogen.

In a particularly preferred embodiment, the present invention provides a compound of formula (I) as described herein, or a pharmaceutically acceptable salt thereof, wherein R⁶ is hydrogen or fluoro.

In one embodiment, the present invention provides a compound of formula (I) as described herein, or a pharmaceutically acceptable salt thereof, wherein R⁷ is C₁-C₆-alkoxy, halo-C₁-C₆-alkoxy, cyano-C₁-C₆-alkoxy or hydroxy-C₂-C₆-alkynyloxy.

In a preferred embodiment, the present invention provides a compound of formula (I) as described herein, or a pharmaceutically acceptable salt thereof, wherein R⁷ is C₁-C₆-alkoxy or cyano-C₁-C₆-alkoxy.

In a particularly preferred embodiment, the present invention provides a compound of formula (I) as described herein, or a pharmaceutically acceptable salt thereof, wherein R⁷ is methoxy or cyanomethoxy.

In a preferred embodiment, the present invention provides a compound of formula (I) as described herein, or a pharmaceutically acceptable salt thereof, wherein R⁸ is hydrogen or halogen.

In a particularly preferred embodiment, the present invention provides a compound of formula (I) as described herein, or a pharmaceutically acceptable salt thereof, wherein R⁸ is hydrogen or fluoro.

In a preferred embodiment, the present invention provides a compound of formula (I) as described herein, or a pharmaceutically acceptable salt thereof, wherein R⁹ is hydrogen.

In a preferred embodiment, the present invention provides a compound of formula (I) as described herein, or a pharmaceutically acceptable salt thereof, wherein R¹⁰ is hydrogen.

In one embodiment, the present invention provides a compound of formula (I) as described herein, or a pharmaceutically acceptable salt thereof, wherein R¹¹ is hydrogen or halogen.

In a preferred embodiment, the present invention provides a compound of formula (I) as described herein, or a pharmaceutically acceptable salt thereof, wherein R¹¹ is hydrogen.

In a preferred embodiment, the present invention provides a compound of formula (I) as described herein, or a pharmaceutically acceptable salt thereof, wherein R¹² is C₁-C₂-alkyl substituted with R¹⁷, R¹⁸, R¹⁹, R²⁰ or R²¹, or a combination thereof.

In a preferred embodiment, the present invention provides a compound of formula (I) as described herein, or a pharmaceutically acceptable salt thereof, wherein R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl or C₁-C₆-alkylsulfonyl.

In a particularly preferred embodiment, the present invention provides a compound of formula (I) as described herein, or a pharmaceutically acceptable salt thereof, wherein R¹³ is hydrogen or hydroxy.

In a preferred embodiment, the present invention provides a compound of formula (I) as described herein, or a pharmaceutically acceptable salt thereof, wherein R¹⁴ is hydrogen.

In a preferred embodiment, the present invention provides a compound of formula (I) as described herein, or a pharmaceutically acceptable salt thereof, wherein R¹⁵ is hydrogen.

In a preferred embodiment, the present invention provides a compound of formula (I) as described herein, or a pharmaceutically acceptable salt thereof, wherein R¹⁶ is hydrogen.

In one embodiment, the present invention provides a compound of formula (I) as described herein, or a pharmaceutically acceptable salt thereof, wherein R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-.

In a preferred embodiment, the present invention provides a compound of formula (I) as described herein, or a pharmaceutically acceptable salt thereof, wherein R¹⁷ is C₁-C₆-alkyl-NH— or (C₁-C₆-alkyl)₂N—.

In a particularly preferred embodiment, the present invention provides a compound of formula (I) as described herein, or a pharmaceutically acceptable salt thereof, wherein R¹⁷ is methylamino or dimethylamino.

In a preferred embodiment, the present invention provides a compound of formula (I) as described herein, or a pharmaceutically acceptable salt thereof, wherein R¹⁸ is hydrogen or amino.

In a particularly preferred embodiment, the present invention provides a compound of formula (I) as described herein, or a pharmaceutically acceptable salt thereof, wherein R¹⁸ is hydrogen.

In a preferred embodiment, the present invention provides a compound of formula (I) as described herein, or a pharmaceutically acceptable salt thereof, wherein R¹⁹ is hydrogen.

In a preferred embodiment, the present invention provides a compound of formula (I) as described herein, or a pharmaceutically acceptable salt thereof, wherein R²⁰ is hydrogen.

In a preferred embodiment, the present invention provides a compound of formula (I) as described herein, or a pharmaceutically acceptable salt thereof, wherein R²¹ is hydrogen.

In a preferred embodiment, the present invention provides a compound of formula (I) as described herein, or a pharmaceutically acceptable salt thereof, wherein A is a monoclic C₂-C₉-heterocycloalkyl ring.

In a particularly preferred embodiment, the present invention provides a compound of formula (I) as described herein, or a pharmaceutically acceptable salt thereof, wherein A is piperazinyl or piperidyl, in particular piperazin-1-yl or 1-piperidyl.

In one embodiment, the present invention provides a compound of formula (I) as described herein, or a pharmaceutically acceptable salt thereof, wherein B is C₁-C₁₃-heteroaryl or C₂-C₉-heterocycloalkyl.

In a preferred embodiment, the present invention provides a compound of formula (I) as described herein, or a pharmaceutically acceptable salt thereof, wherein B is C₂-C₉-heterocycloalkyl.

In a particularly preferred embodiment, the present invention provides a compound of formula (I) as described herein, or a pharmaceutically acceptable salt thereof, wherein B is piperazinyl, pyrrolidinyl or azetidinyl, in particular piperazin-1-yl, pyrrolidin-2-yl or azetidin-3-yl.

In a preferred embodiment, the present invention provides a compound of formula (I) as described herein, or a pharmaceutically acceptable salt thereof, wherein L¹ is a covalent bond, carbonyl, —N(C₁-C₆-alkyl)- or —NH—C(O)—.

In a particularly preferred embodiment, the present invention provides a compound of formula (I) as described herein, or a pharmaceutically acceptable salt thereof, wherein L¹ is a covalent bond, carbonyl or —NH—C(O)—.

In one embodiment, the present invention provides a compound of formula (I) as described herein, or a pharmaceutically acceptable salt thereof, wherein L² is a covalent bond, —C₁-C₆-alkyl-, carbonyl, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-.

In a preferred embodiment, the present invention provides a compound of formula (I) as described herein, or a pharmaceutically acceptable salt thereof, wherein L² is carbonyl or —C₁-C₆-alkyl-NH—C(O)—.

In a particularly preferred embodiment, the present invention provides a compound of formula (I) as described herein, or a pharmaceutically acceptable salt thereof, wherein L² is carbonyl or —CH₂NH—C(O)—.

In one embodiment, the present invention provides a compound of formula (I) as described herein or a pharmaceutically acceptable salt thereof, wherein:
A is a monocyclic C₂-C₉-heterocycloalkyl ring;
R¹ is hydrogen, (C₁-C₆-alkyl)₂N—, hydroxy, carboxy, a group

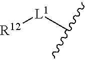

or a group

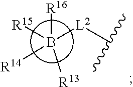

R² is hydrogen or hydroxy;
R³ is hydrogen, halogen or CN;
R⁵, R⁶ and R⁸ are each independently hydrogen or halogen;
R⁷ is C₁-C₆-alkoxy, halo-C₁-C₆-alkoxy, cyano-C₁-C₆-alkoxy or hydroxy-C₂-C₆-alkynyloxy;
R¹¹ is hydrogen or halogen;
R¹² is C₁-C₆-alkyl substituted with R¹⁷, R¹⁸, R¹⁹, R²⁰ or R²¹, or a combination thereof;
R⁴, R⁹, R¹⁰, R¹⁴, R¹⁵ and R¹⁶ are all hydrogen;
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl, C₁-C₆-alkylsulfonyl, C₁-C₁₃-heteroaryl or C₂-C₉-heterocycloalkyl-C₁-C₆-alkyl-;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen, hydroxy or amino;
R¹⁹, R²⁰ and R²¹ are each independently hydrogen or hydroxy;
B is C₁-C₁₃-heteroaryl or C₂-C₉-heterocycloalkyl;
L¹ is a covalent bond, carbonyl, —N(C₁-C₆-alkyl)-, —NH—C(O)— or —N(C₁-C₆-alkyl)-; and
L² is a covalent bond, —C₁-C₆-alkyl-, carbonyl, —C₁-C₆-alkyl-C(O)—, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-.

In one embodiment, the present invention provides a compound of formula (I) as described herein, or a pharmaceutically acceptable salt thereof, wherein:
A is a monocyclic C₂-C₉-heterocycloalkyl ring;
R¹ is hydrogen, (C₁-C₆-alkyl)₂N—, hydroxy, carboxy, a group

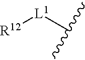

or a group

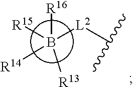

R² is hydrogen or hydroxy;
R³ is hydrogen, halogen or CN;
R⁵, R⁶ and R⁸ are each independently hydrogen or halogen;
R⁷ is C₁-C₆-alkoxy, halo-C₁-C₆-alkoxy, cyano-C₁-C₆-alkoxy or hydroxy-C₂-C₆-alkynyloxy;
R¹² is C₁-C₆-alkyl substituted with R¹⁷, R¹⁸, R¹⁹, R²⁰ or R²¹, or a combination thereof;
R⁴, R⁹, R¹⁰, R¹¹, R¹⁴, R¹⁵, R¹⁶, R¹⁹, R²⁰ and R²¹ are all hydrogen;
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl or C₁-C₆-alkylsulfonyl;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen or amino;
B is C₁-C₁₃-heteroaryl or C₂-C₉-heterocycloalkyl;
L¹ is a covalent bond, carbonyl, —N(C₁-C₆-alkyl)- or —NH—C(O)—; and
L² is a covalent bond, —C₁-C₆-alkyl-, carbonyl, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-.

In a preferred embodiment, the present invention provides a compound of formula (I) as described herein, or a pharmaceutically acceptable salt thereof, wherein:
A is a monocyclic C₂-C₉-heterocycloalkyl ring;
R¹ is a group

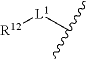

or a group

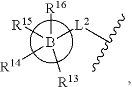

R² is hydrogen or hydroxy;
R³ is halogen;
R⁵, R⁶ and R⁸ are each independently hydrogen or halogen;
R⁷ is C₁-C₆-alkoxy or cyano-C₁-C₆-alkoxy;
R¹² is C₁-C₆-alkyl substituted with R¹⁷, R¹⁸, R¹⁹, R²⁰ or R²¹, or a combination thereof;
R⁴, R⁹, R¹⁰, R¹¹, R¹⁴, R¹⁵, R¹⁶, R¹⁸, R¹⁹, R²⁰ and R²¹ are all hydrogen;
R¹³ is hydrogen or hydroxy;
R¹⁷ is C₁-C₆-alkyl-NH— or (C₁-C₆-alkyl)₂N—;
B is C₂-C₉-heterocycloalkyl;
L¹ is a covalent bond, carbonyl or —NH—C(O)—; and
L² is a carbonyl or —C₁-C₆-alkyl-NH—C(O)—.

In a particularly preferred embodiment, the present invention provides a compound of formula (I) as described herein, or a pharmaceutically acceptable salt thereof, wherein:
A is piperazinyl or piperidyl, in particular piperazin-1-yl or 1-piperidyl;
R¹ is a group

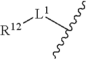

or a group

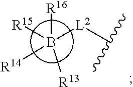

R² is hydrogen or hydroxy;
R³ is chloro;
R⁵ is hydrogen, fluoro or chloro;
R⁶ and R⁸ are each independently hydrogen or fluoro;
R⁷ is methoxy or cyanomethoxy;
R¹² is C₁-C₂-alkyl substituted with R¹⁷, R¹⁸, R¹⁹, R²⁰ or R²¹, or a combination thereof;
R⁴, R⁹, R¹⁰, R¹¹, R¹⁴, R¹⁵, R¹⁶, R¹⁸, R¹⁹, R²⁰ and R²¹ are all hydrogen;
R¹³ is hydrogen or hydroxy;
R¹⁷ is methylamino or dimethylamino;
B is piperazinyl, pyrrolidinyl or azetidinyl, in particular piperazin-1-yl, pyrrolidin-2-yl or azetidin-3-yl;
L¹ is a covalent bond, carbonyl or —NH—C(O)—; and
L² is carbonyl or —CH₂NH—C(O)—.

In one embodiment, the present invention provides a compound of formula (I) as described herein, or a pharmaceutically acceptable salt thereof, wherein:
A is a monocyclic C₂-C₉-heterocycloalkyl ring;
R¹ is hydrogen, (C₁-C₆-alkyl)₂N—, hydroxy, carboxy, a group

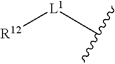

or a group;

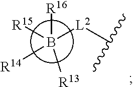

R² is hydrogen or hydroxy;
R¹² is C₁-C₆-alkyl substituted with R¹⁷, R¹⁸, R¹⁹, R²⁰ or R²¹, or a combination thereof;
R¹⁴, R¹⁵, R¹⁶, R¹⁹, R²⁰ and R²¹ are all hydrogen;
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl or C₁-C₆-alkylsulfonyl;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen or amino;
B is C₁-C₁₃-heteroaryl or C₂-C₉-heterocycloalkyl;
L¹ is a covalent bond, carbonyl, —N(C₁-C₆-alkyl)- or —NH—C(O)—; and
L² is a covalent bond, —C₁-C₆-alkyl-, carbonyl, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-.

In a preferred embodiment, the present invention provides a compound of formula (I) as described herein, or a pharmaceutically acceptable salt thereof, wherein:
A is a monocyclic C₂-C₉-heterocycloalkyl ring;
R¹ is a group

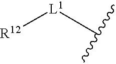

or a group

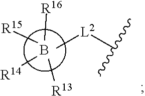

R² is hydrogen or hydroxy;
R¹² is C₁-C₆-alkyl substituted with R¹⁷, R¹⁸, R¹⁹, R²⁰ or R²¹, or a combination thereof;
R¹³ is hydrogen or hydroxy;
R¹⁴, R¹⁵, R¹⁶, R¹⁸, R¹⁹, R²⁰ and R²¹ are all hydrogen;
R¹⁷ is C₁-C₆-alkyl-NH— or (C₁-C₆-alkyl)₂N—;
B is C₂-C₉-heterocycloalkyl;
L¹ is a covalent bond, carbonyl or —NH—C(O)—; and
L² is a carbonyl or —C₁-C₆-alkyl-NH—C(O)—.

In a particularly preferred embodiment, the present invention provides a compound of formula (I) as described herein, or a pharmaceutically acceptable salt thereof, wherein:
A is piperazinyl or piperidyl, in particular piperazin-1-yl or 1-piperidyl;
R¹ is a group

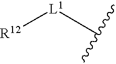

or a group

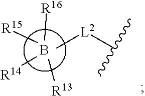

R² is hydrogen or hydroxy;
R¹² is C₁-C₂-alkyl substituted with R¹⁷, R¹⁸, R¹⁹, R²⁰ or R²¹, or a combination thereof;
R¹³ is hydrogen or hydroxy;
R¹⁴, R¹⁵, R¹⁶, R¹⁸, R¹⁹, R²⁰ and R²¹ are all hydrogen;
R¹⁷ is methylamino or dimethylamino;
B is piperazinyl, pyrrolidinyl or azetidinyl, in particular piperazin-1-yl, pyrrolidin-2-yl or azetidin-3-yl;
L¹ is a covalent bond, carbonyl or —NH—C(O)—; and
L² is carbonyl or —CH₂NH—C(O)—.

In one embodiment, the present invention provides a compound of formula (I) as described herein, or a pharmaceutically acceptable salt thereof, wherein:
R³ is hydrogen, halogen or CN; and
R⁴, R¹⁰ and R¹¹ are all hydrogen.

In a preferred embodiment, the present invention provides a compound of formula (I) as described herein, or a pharmaceutically acceptable salt thereof, wherein:
R³ is halogen; and
R⁴, R¹⁰ and R¹¹ are all hydrogen.

In a particularly preferred embodiment, the present invention provides a compound of formula (I) as described herein, or a pharmaceutically acceptable salt thereof, wherein:
R³ is chloro; and
R⁴, R¹⁰ and R¹¹ are all hydrogen.

In one embodiment, the present invention provides a compound of formula (I) as described herein, or a pharmaceutically acceptable salt thereof, wherein:
R⁵, R⁶ and R⁸ are each independently hydrogen or halogen;
R⁷ is C₁-C₆-alkoxy, halo-C₁-C₆-alkoxy, cyano-C₁-C₆-alkoxy or hydroxy-C₂-C₆-alkynyloxy; and
R⁹ is hydrogen.

In a preferred embodiment, the present invention provides a compound of formula (I) as described herein, or a pharmaceutically acceptable salt thereof, wherein:
R⁵, R⁶ and R⁸ are each independently hydrogen or halogen;
R⁷ is C₁-C₆-alkoxy or cyano-C₁-C₆-alkoxy; and
R⁹ is hydrogen.

In a particularly preferred embodiment, the present invention provides a compound of formula (I) as described herein, or a pharmaceutically acceptable salt thereof, wherein:
R⁵, R⁶ and R⁸ are each independently hydrogen or halogen;
R⁷ is C₁-C₆-alkoxy or cyano-C₁-C₆-alkoxy; and
R⁹ is hydrogen.

In a further particularly preferred embodiment, the present invention provides a compound of formula (I) as described herein, or a pharmaceutically acceptable salt thereof, wherein the group

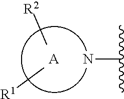

is:

4-(2-aminoethyl)-1-piperidyl; 4-[2-(dimethylamino)ethyl]piperazin-1-yl; 4-(aminomethyl)-1-piperidyl; morpholino; (4-hydroxy-1-piperidyl); 4-(2-hydroxyethyl)piperazin-1-yl; (4-methylpiperazin-1-yl); 1-piperidyl; aziridin-1-yl; (3-carboxypiperazin-1-yl); 3-(dimethylamino)pyrrolidin-1-yl; 4-[2-(methylamino)ethylcarbamoyl]-1-piperidyl; 4-[2-(aminomethyl)morpholine-4-carbonyl]-1-piperidyl; 4-[(dimethylamino)methyl]-1-piperidyl; 4-[2-(methylamino)acetyl]piperazin-1-yl; (4-piperazin-1-yl-1-piperidyl); 4-[3-(hydroxymethyl)piperazine-1-carbonyl]-1-piperidyl; 4-[2-(methylamino)ethyl]piperazin-1-yl; 4-(piperazin-2-ylmethoxycarbonyl)-1-piperidyl; 4-[2-(2-hydroxyethylamino)ethyl]piperazin-1-yl; 4-[2-(hydroxymethyl)piperazine-1-carbonyl]-1-piperidyl; 4-[[(3R)-pyrrolidin-3-yl]carbamoyl]-1-piperidyl; 4-(1H-tetrazol-5-yl)-1-piperidyl; 4-(azetidin-3-ylmethylcarbamoyl)-1-piperidyl; 4-(3-carboxypiperazine-1-carbonyl)-1-piperidyl; 4-[(3S)-3-carboxypiperazine-1-carbonyl]-1-piperidyl; 4-[(3R)-3-carboxypiperazine-1-carbonyl]-1-piperidyl; 4-[(3S)-3-carboxypiperazine-1-carbonyl]piperazin-1-yl; 4-[3-(dimethylamino)propyl]piperazin-1-yl; 4-(2-pyrrolidin-1-ylethyl)piperazin-1-yl; 4-(1H-imidazol-5-yl)-1-piperidyl; 4-(1,2,4-triazol-4-yl)-1-piperidyl; 4-(4-methyl-1,2,4-triazol-3-yl)-1-piperidyl; 4-(3-amino-2-hydroxy-propanoyl)piperazin-1-yl; (4-pyrrolidin-3-ylpiperazin-1-yl); 4-[2-(azetidin-1-yl)ethyl]piperazin-1-yl; 4-[2-(cyclopropylamino)ethyl]piperazin-1-yl; 4-[(pyridine-4-carbonylamino)methyl]-1-piperidyl; 4-(1H-imidazol-5-ylmethyl)piperazin-1-yl; 4-(azetidin-3-yl)piperazin-1-yl; (4-pyrimidin-2-ylpiperazin-1-yl); 4-(4-pyridyl)-1-piperidyl; 4-[2-(dimethylamino)ethyl]-4-hydroxy-1-piperidyl; 4-[(2S,4R)-4-hydroxypyrrolidine-2-carbonyl]piperazin-1-yl; 4-[2-(3-carboxypyrrolidin-1-yl)ethyl]piperazin-1-yl; 4-[2-(dimethylamino)acetyl]piperazin-1-yl; 4-[(3R)-3-(hydroxymethyl)piperazine-1-carbonyl]piperazin-1-yl; 4-[(3S)-3-(hydroxymethyl)piperazine-1-carbonyl]piperazin-1-yl; 4-[2-(3-carbamoylpyrrolidin-1-yl)ethyl]piperazin-1-yl; 4-[2-[3-(hydroxymethyl)pyrrolidin-1-yl]ethyl]piperazin-1-yl; 4-[(3R)-3-(hydroxymethyl)piperazine-1-carbonyl]-1-piperidyl; 4-[2-[3-(hydroxymethyl)azetidin-1-yl]ethyl]piperazin-1-yl; 4-[(3S)-3-(hydroxymethyl)piperazine-1-carbonyl]-1-piperidyl; 4-[2-[2-(hydroxymethyl)pyrrolidin-1-yl]ethyl]piperazin-1-yl; 4-[2-(3-carbamoylazetidin-1-yl)ethyl]piperazin-1-yl; 4-[2-(3-hydroxypyrrolidin-1-yl)ethyl]piperazin-1-yl; 4-[2-(3-hydroxyazetidin-1-yl)ethyl]piperazin-1-yl; 4-[2-(3-methoxyazetidin-1-yl)ethyl]piperazin-1-yl; 4-[(3-amino-2-hydroxy-propyl)carbamoyl]-1-piperidyl; 4-[2-(3-methylsulfonylazetidin-1-yl)ethyl]piperazin-1-yl; 4-[2-[3-[(dimethylamino)methyl]azetidin-1-yl]ethyl]piperazin-1-yl; 4-[2-[3-(dimethylamino)pyrrolidin-1-yl]ethyl]piperazin-1-yl; 4-[2-[4-(dimethylamino)-1-piperidyl]ethyl]piperazin-1-yl; 4-[2-[3-(dimethylamino)azetidin-1-yl]ethyl]piperazin-1-yl; or 4-[2-(3-methylsulfonylpyrrolidin-1-yl)ethyl]piperazin-1-yl.

In one embodiment, the present invention provides a compound of formula (I) as described herein, or a pharmaceutically acceptable salt thereof, wherein the compound of formula (I) is selected from:
[4-(2-aminoethyl)-1-piperidyl]-[2-chloro-4-[[3-(2,3-difluoro-4-methoxy-phenyl)imidazo[1,2-a]pyrazin-8-yl]amino]phenyl]methanone;
[2-chloro-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
[4-(aminomethyl)-1-piperidyl]-[2-chloro-4-[[3-(3-fluoro-4-methoxy-phenyl)imidazo[1,2-a]pyrazin-8-yl]amino]phenyl]methanone;
[4-(aminomethyl)-1-piperidyl]-[2-bromo-4-[[3-[4-(difluoromethoxy)phenyl]imidazo[1,2-a]pyrazin-8-yl]amino]phenyl]methanone;
[4-(aminomethyl)-1-piperidyl]-[2-chloro-4-[[3-(2,3-difluoro-4-methoxy-phenyl)imidazo[1,2-a]pyrazin-8-yl]amino]phenyl]methanone;
[4-(aminomethyl)-1-piperidyl]-[4-[[3-[4-(difluoromethoxy)phenyl]imidazo[1,2-a]pyrazin-8-yl]amino]-2-iodo-phenyl]methanone;
[4-[[3-(3-chloro-4-methoxy-phenyl)imidazo[1,2-a]pyrazin-8-yl]amino]phenyl]-morpholino-methanone;
[4-[[3-(4-methoxyphenyl)imidazo[1,2-a]pyrazin-8-yl]amino]phenyl]-morpholino-methanone;
[4-[[3-(3-chloro-4-methoxy-phenyl)imidazo[1,2-a]pyrazin-8-yl]amino]phenyl]-(4-hydroxy-1-piperidyl)methanone;
[4-(2-hydroxyethyl)piperazin-1-yl]-[4-[[3-(4-methoxyphenyl)imidazo[1,2-a]pyrazin-8-yl]amino]phenyl]methanone;
[4-[[3-(4-methoxyphenyl)imidazo[1,2-a]pyrazin-8-yl]amino]phenyl]-(4-methylpiperazin-1-yl)methanone;
2-[4-(aminomethyl)piperidine-1-carbonyl]-5-[[3-[4-(difluoromethoxy)phenyl]imidazo[1,2-a]pyrazin-8-yl]amino]benzonitrile;
[4-[[3-(3-chloro-4-methoxy-phenyl)imidazo[1,2-a]pyrazin-8-yl]amino]phenyl]-(1-piperidyl)methanone;
aziridin-1-yl-[2-chloro-4-[[3-(2,3-difluoro-4-methoxy-phenyl)imidazo[1,2-a]pyrazin-8-yl]amino]phenyl]methanone;
4-[2-chloro-4-[[3-(2,3-difluoro-4-methoxy-phenyl)imidazo[1,2-a]pyrazin-8-yl]amino]benzoyl]piperazine-2-carboxylic acid;
[4-[[3-(3-chloro-4-methoxy-phenyl)imidazo[1,2-a]pyrazin-8-yl]amino]phenyl]-(4-methylpiperazin-1-yl)methanone;
[3-(dimethylamino)pyrrolidin-1-yl]-[4-[[3-(4-methoxyphenyl)imidazo[1,2-a]pyrazin-8-yl]amino]phenyl]methanone;
2-[4-[8-[4-[4-(aminomethyl)piperidine-1-carbonyl]-3-chloro-anilino]imidazo[1,2-a]pyrazin-3-yl]-2,3-difluoro-phenoxy]acetonitrile;
2-[4-[8-[3-chloro-4-[4-[2-(dimethylamino)ethyl]piperazine-1-carbonyl]anilino]imidazo[1,2-a]pyrazin-3-yl]-2,3-difluoro-phenoxy]acetonitrile;
1-[2-chloro-4-[[3-(3-fluoro-4-methoxy-phenyl)imidazo[1,2-a]pyrazin-8-yl]amino]benzoyl]-N-[2-(methylamino)ethyl]piperidine-4-carboxamide;
[4-(aminomethyl)-1-piperidyl]-[2-bromo-4-[[3-(3-fluoro-4-methoxy-phenyl)imidazo[1,2-a]pyrazin-8-yl]amino]phenyl]methanone;
5 [4-[2-(aminomethyl)morpholine-4-carbonyl]-1-piperidyl]-[2-chloro-4-[[3-(3-fluoro-4-methoxy-phenyl)imidazo[1,2-a]pyrazin-8-yl]amino]phenyl]methanone;
2-[3-chloro-4-[8-[3-chloro-4-[4-[(dimethylamino)methyl]piperidine-1-carbonyl]anilino]imidazo[1,2-a]pyrazin-3-yl]-2-fluoro-phenoxy]acetonitrile;
1-[4-[2-chloro-4-[[3-(3-fluoro-4-methoxy-phenyl)imidazo[1,2-a]pyrazin-8-yl]amino]benzoyl]piperazin-1-yl]-2-(methylamino)ethanone;
[2-chloro-4-[[3-[4-(difluoromethoxy)phenyl]imidazo[1,2-a]pyrazin-8-yl]amino]phenyl]-(4-piperazin-1-yl-1-piperidyl)methanone;
2-[5-chloro-4-[8-[3-chloro-4-[4-[3-(hydroxymethyl)piperazine-1-carbonyl]piperidine-1-carbonyl]anilino]imidazo[1,2-a]pyrazin-3-yl]-2-fluoro-phenoxy]acetonitrile;
[2-chloro-4-[[3-(3-fluoro-4-methoxy-phenyl)imidazo[1,2-a]pyrazin-8-yl]amino]phenyl]-[4-[2-(methylamino)ethyl]piperazin-1-yl]methanone;
piperazin-2-ylmethyl 1-[2-chloro-4-[[3-[4-(cyanomethoxy)-2,3-difluoro-phenyl]imidazo[1,2-a]pyrazin-8-yl]amino]benzoyl]piperidine-4-carboxylate;
[2-chloro-4-[[3-(3-fluoro-4-methoxy-phenyl)imidazo[1,2-a]pyrazin-8-yl]amino]phenyl]-[4-[2-(2-hydroxyethylamino)ethyl]piperazin-1-yl]methanone;
2-[4-[8-[3-chloro-4-[4-[2-(hydroxymethyl)piperazine-1-carbonyl]piperidine-1-carbonyl]anilino]imidazo[1,2-a]pyrazin-3-yl]-2,3-difluoro-phenoxy]acetonitrile;
1-[2-chloro-4-[[3-(2,3-difluoro-4-methoxy-phenyl)imidazo[1,2-a]pyrazin-8-yl]amino]benzoyl]-N-[(3R)-pyrrolidin-3-yl]piperidine-4-carboxamide;
1-[2-chloro-4-[[3-(3-fluoro-4-methoxy-phenyl)imidazo[1,2-a]pyrazin-8-yl]amino]benzoyl]-N-[(3R)-pyrrolidin-3-yl]piperidine-4-carboxamide;
2-[4-[8-[3-chloro-4-[4-(1H-tetrazol-5-yl)piperidine-1-carbonyl]anilino]imidazo[1,2-a]pyrazin-3-yl]-2,3-difluoro-phenoxy]acetonitrile;
2-[4-[8-[3-bromo-4-[4-[2-(dimethylamino)ethyl]piperazine-1-carbonyl]anilino]imidazo[1,2-a]pyrazin-3-yl]-2,3-difluoro-phenoxy]acetonitrile;
N-(azetidin-3-ylmethyl)-1-[2-chloro-4-[[3-(3-fluoro-4-methoxy-phenyl)imidazo[1,2-a]pyrazin-8-yl]amino]benzoyl]piperidine-4-carboxamide;
1-[2-bromo-4-[[3-(3-fluoro-4-methoxy-phenyl)imidazo[1,2-a]pyrazin-8-yl]amino]benzoyl]-N-[2-(methylamino)ethyl]piperidine-4-carboxamide;
4-[1-[2-chloro-4-[[3-(3-fluoro-4-methoxy-phenyl)imidazo[1,2-a]pyrazin-8-yl]amino]benzoyl]piperidine-4-carbonyl]piperazine-2-carboxylic acid;
(2S)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic acid;
(2R)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic acid;
(2S)-4-[4-[4-[[3-[4-(difluoromethoxy)phenyl]imidazo[1,2-a]pyrazin-8-yl]amino]-2-methyl-benzoyl]piperazine-1-carbonyl]piperazine-2-carboxylic acid;
1-[4-[2-chloro-4-[[3-(2,3-difluoro-4-methoxy-phenyl)imidazo[1,2-a]pyrazin-8-yl]amino]benzoyl]piperazin-1-yl]-2-(methylamino)ethanone;
2-[3-chloro-4-[8-[3-chloro-4-[4-[3-(dimethylamino)propyl]piperazine-1-carbonyl]anilino]imidazo[1,2-a]pyrazin-3-yl]-2-fluoro-phenoxy]acetonitrile;
[4-(aminomethyl)-1-piperidyl]-[2-chloro-4-[[3-(2,3-difluoro-4-prop-2-ynoxy-phenyl)imidazo[1,2-a]pyrazin-8-yl]amino]phenyl]methanone;
2-[3-chloro-4-[8-[3-chloro-4-[4-(2-pyrrolidin-1-ylethyl)piperazine-1-carbonyl]anilino]imidazo[1,2-a]pyrazin-3-yl]-2-fluoro-phenoxy]acetonitrile;
[2-chloro-4-[[3-(2-chloro-3-fluoro-4-methoxy-phenyl)imidazo[1,2-a]pyrazin-8-yl]amino]phenyl]-[4-[2-(dimethylamino)ethyl]piperazin-1-yl]methanone;
2-[4-[8-[3-chloro-4-[4-[2-(dimethylamino)ethyl]piperazine-1-carbonyl]anilino]imidazo[1,2-a]pyrazin-3-yl]-2,3-difluoro-phenoxy]propanenitrile;
[2-chloro-4-[[3-[4-(difluoromethoxy)phenyl]imidazo[1,2-a]pyrazin-8-yl]amino]phenyl]-[4-[2-(dimethylamino)ethyl]piperazin-1-yl]methanone;
2-[4-[8-[3-chloro-4-[4-(1H-imidazol-5-yl)piperidine-1-carbonyl]anilino]imidazo[1,2-a]pyrazin-3-yl]-2,3-difluoro-phenoxy]acetonitrile;
2-[4-[8-[3-chloro-4-[4-(1,2,4-triazol-4-yl)piperidine-1-carbonyl]anilino]imidazo[1,2-a]pyrazin-3-yl]-2,3-difluoro-phenoxy]acetonitrile;
2-[4-[8-[3-chloro-4-[4-(4-methyl-1,2,4-triazol-3-yl)piperidine-1-carbonyl]anilino]imidazo[1,2-a]pyrazin-3-yl]-2,3-difluoro-phenoxy]acetonitrile;
2-[4-[8-[3-chloro-4-(morpholine-4-carbonyl)anilino]imidazo[1,2-a]pyrazin-3-yl]-2,3-difluoro-phenoxy]acetonitrile;
2-[4-[8-[3-chloro-4-[4-[3-(dimethylamino)propyl]piperazine-1-carbonyl]anilino]imidazo[1,2-a]pyrazin-3-yl]-2,3-difluoro-phenoxy]propanenitrile;
[4-(aminomethyl)-1-piperidyl]-[2-chloro-4-[[3-[3-fluoro-4-(4-hydroxybut-2-ynoxy)phenyl]imidazo[1,2-a]pyrazin-8-yl]amino]phenyl]methanone;
3-amino-1-[4-[2-chloro-4-[[3-(3-fluoro-4-methoxy-phenyl)imidazo[1,2-a]pyrazin-8-yl]amino]benzoyl]piperazin-1-yl]-2-hydroxy-propan-1-one;
[2-chloro-4-[[3-(3-fluoro-4-methoxy-phenyl)imidazo[1,2-a]pyrazin-8-yl]amino]phenyl]-[4-[3-(hydroxymethyl)piperazine-1-carbonyl]-1-piperidyl]methanone;
[2-chloro-4-[[3-(3-fluoro-4-methoxy-phenyl)imidazo[1,2-a]pyrazin-8-yl]amino]phenyl]-(4-pyrrolidin-3-ylpiperazin-1-yl)methanone;
[2-bromo-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
1-[2-chloro-4-[[3-[4-(cyanomethoxy)-2,3-difluoro-phenyl]imidazo[1,2-a]pyrazin-8-yl]amino]benzoyl]-N-[2-(methylamino)ethyl]piperidine-4-carboxamide;
2-[3-chloro-4-[8-[3-chloro-4-[4-[2-(hydroxymethyl)piperazine-1-carbonyl]piperidine-1-carbonyl]anilino]imidazo[1,2-a]pyrazin-3-yl]-2-fluoro-phenoxy]acetonitrile;
[4-[2-(azetidin-1-yl)ethyl]piperazin-1-yl]-[2-chloro-4-[[3-(3-fluoro-4-methoxy-phenyl)imidazo[1,2-a]pyrazin-8-yl]amino]phenyl]methanone;
[2-chloro-4-[[3-(3-fluoro-4-methoxy-phenyl)imidazo[1,2-a]pyrazin-8-yl]amino]phenyl]-[4-[2-(cyclopropylamino)ethyl]piperazin-1-yl]methanone;
N-[[1-[2-chloro-4-[[3-[4-(cyanomethoxy)-2,3-difluoro-phenyl]imidazo[1,2-a]pyrazin-8-yl]amino]benzoyl]-4-piperidyl]methyl]pyridine-4-carboxamide;
[2-chloro-4-[[3-(3-fluoro-4-methoxy-phenyl)imidazo[1,2-a]pyrazin-8-yl]amino]phenyl]-[4-(1H-imidazol-5-ylmethyl)piperazin-1-yl]methanone;
[4-(azetidin-3-yl)piperazin-1-yl]-[2-chloro-4-[[3-(3-fluoro-4-methoxy-phenyl)imidazo[1,2-a]pyrazin-8-yl]amino]phenyl]methanone;
2-[4-[8-[3-chloro-4-(4-pyrimidin-2-ylpiperazine-1-carbonyl)anilino]imidazo[1,2-a]pyrazin-3-yl]-2,3-difluoro-phenoxy]acetonitrile;
2-[4-[8-[3-chloro-4-[4-(4-pyridyl)piperidine-1-carbonyl]anilino]imidazo[1,2-a]pyrazin-3-yl]-2,3-difluoro-phenoxy]acetonitrile;
[2-chloro-4-[[3-(3-fluoro-4-methoxy-phenyl)imidazo[1,2-a]pyrazin-8-yl]amino]phenyl]-[4-[2-(dimethylamino)ethyl]-4-hydroxy-1-piperidyl]methanone;
[4-[[3-(2,3-difluoro-4-methoxy-phenyl)imidazo[1,2-a]pyrazin-8-yl]amino]-2,6-difluoro-phenyl]-[4-[(2S,4R)-4-hydroxypyrrolidine-2-carbonyl]piperazin-1-yl]methanone;
[2-chloro-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
2-[4-[8-[4-[4-[2-(azetidin-1-yl)ethyl]piperazine-1-carbonyl]-3-chloro-anilino]imidazo[1,2-a]pyrazin-3-yl]-2,3-difluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylic acid;
2-[4-[8-[3-chloro-4-[4-[2-(dimethylamino)acetyl]piperazine-1-carbonyl]anilino]imidazo[1,2-a]pyrazin-3-yl]-2,3-difluoro-phenoxy]acetonitrile;
[2-chloro-4-[[3-(2,3-difluoro-4-methoxy-phenyl)imidazo[1,2-a]pyrazin-8-yl]amino]phenyl]-[4-[(2S,4R)-4-hydroxypyrrolidine-2-carbonyl]piperazin-1-yl]methanone;
2-[4-[8-[3-chloro-4-[4-[(3R)-3-(hydroxymethyl)piperazine-1-carbonyl]piperazine-1-carbonyl]anilino]imidazo[1,2-a]pyrazin-3-yl]-2,3-difluoro-phenoxy]acetonitrile;
2-[4-[8-[3-chloro-4-[4-[(3S)-3-(hydroxymethyl)piperazine-1-carbonyl]piperazine-1-carbonyl]anilino]imidazo[1,2-a]pyrazin-3-yl]-2,3-difluoro-phenoxy]acetonitrile;
2-[4-[8-[3-chloro-4-[4-[(2S,4R)-4-hydroxypyrrolidine-2-carbonyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxamide;
2-[4-[8-[3-chloro-4-[4-[2-[3-(hydroxymethyl)pyrrolidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[(3R)-3-(hydroxymethyl)piperazine-1-carbonyl]piperidine-1-carbonyl]anilino]imidazo[1,2-a]pyrazin-3-yl]-2,3-difluoro-phenoxy]acetonitrile;
2-[4-[8-[3-chloro-4-[4-[2-(methylamino)ethyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(3R)-3-(hydroxymethyl)piperazine-1-carbonyl]piperidine-1-carbonyl]anilino]imidazo[1,2-a]pyrazin-3-yl]-2-fluoro-phenoxy]acetonitrile;
2-[4-[8-[3-chloro-4-[4-[2-[3-(hydroxymethyl)azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[(3S)-3-(hydroxymethyl)piperazine-1-carbonyl]piperidine-1-carbonyl]anilino]imidazo[1,2-a]pyrazin-3-yl]-2,3-difluoro-phenoxy]acetonitrile;
2-[4-[8-[3-chloro-4-[4-[2-[2-(hydroxymethyl)pyrrolidin-1-yl]ethyl]piperazine-1-carbonyl]anilino]imidazo[1,2-a]pyrazin-3-yl]-2,3-difluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]azetidine-3-carboxamide;
2-[4-[8-[3-chloro-4-[4-[2-(3-hydroxypyrrolidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(3-hydroxyazetidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(methylamino)acetyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(3S)-3-(hydroxymethyl)piperazine-1-carbonyl]piperidine-1-carbonyl]anilino]imidazo[1,2-a]pyrazin-3-yl]-2-fluoro-phenoxy]acetonitrile;
2-[4-[8-[3-chloro-4-[4-[2-(3-methoxyazetidin-1-yl)ethyl]piperazine-1-carbonyl]anilino]imidazo[1,2-a]pyrazin-3-yl]-2,3-difluoro-phenoxy]acetonitrile;
N-(3-amino-2-hydroxy-propyl)-1-[2-chloro-4-[[3-(3-fluoro-4-methoxy-phenyl)imidazo[1,2-a]pyrazin-8-yl]amino]benzoyl]piperidine-4-carboxamide;
2-[4-[8-[3-chloro-4-[4-[2-(3-methylsulfonylazetidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-[(dimethylamino)methyl]azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-(dimethylamino)pyrrolidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[4-(dimethylamino)-1-piperidyl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-(dimethylamino)azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(3-methylsulfonylpyrrolidin-1-yl)ethyl]piperazine-1-carbonyl]anilino]imidazo[1,2-a]pyrazin-3-yl]-2,3-difluoro-phenoxy]acetonitrile;
[4-[[3-[4-(difluoromethoxy)-2,3-difluoro-phenyl]imidazo[1,2-a]pyrazin-8-yl]amino]-2-fluoro-phenyl]-[4-[1-(pyrrolidin-3-ylmethyl)piperidine-4-carbonyl]piperazin-1-yl]methanone;
25 [2-chloro-4-[[3-[4-(difluoromethoxy)-2,3-difluoro-phenyl]imidazo[1,2-a]pyrazin-8-yl]amino]phenyl]-[4-[(2S,4R)-4-hydroxypyrrolidine-2-carbonyl]piperazin-1-yl]methanone;
[2-chloro-4-[[3-[2,3-difluoro-4-(fluoromethoxy)phenyl]imidazo[1,2-a]pyrazin-8-yl]amino]phenyl]-[4-[(2S,4R)-4-hydroxypyrrolidine-2-carbonyl]piperazin-1-yl]methanone;
[2-chloro-4-[[3-(2,3-difluoro-4-methoxy-phenyl)imidazo[1,2-a]pyrazin-8-yl]amino]phenyl]-[4-hydroxy-4-(methylaminomethyl)-1-piperidyl]methanone;
[2-chloro-4-[[3-(3-fluoro-4-methoxy-phenyl)imidazo[1,2-a]pyrazin-8-yl]amino]phenyl]-[4-hydroxy-4-(methylaminomethyl)-1-piperidyl]methanone;
[2-bromo-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
[2-bromo-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]-2-oxo-ethyl]pyrrolidine-3-carboxylic acid;
1-[2-chloro-4-[[3-(3-fluoro-4-methoxy-phenyl)imidazo[1,2-a]pyrazin-8-yl]amino]benzoyl]-N-[(2S,3R,4R,5R)-2,3,4,5,6-pentahydroxyhexyl]piperidine-4-carboxamide;
2-[1-[2-chloro-4-[[3-(3-fluoro-4-methoxy-phenyl)imidazo[1,2-a]pyrazin-8-yl]amino]benzoyl]-4-hydroxy-4-piperidyl]-N,N-dimethyl-acetamide;
2-[4-[8-[3-chloro-4-[4-[2-[3-(1H-tetrazol-5-yl)azetidin-1-yl]ethyl]piperazine-1-carbonyl]anilino]imidazo[1,2-a]pyrazin-3-yl]-2,3-difluoro-phenoxy]acetonitrile; and
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-2-carboxylic acid.

In a preferred embodiment, the present invention provides a compound of formula (I) as described herein, or a pharmaceutically acceptable salt thereof, wherein the compound of formula (I) is selected from:
[2-chloro-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
1-[2-chloro-4-[[3-(3-fluoro-4-methoxy-phenyl)imidazo[1,2-a]pyrazin-8-yl]amino]benzoyl]-N-[2-(methylamino)ethyl]piperidine-4-carboxamide;
1-[4-[2-chloro-4-[[3-(3-fluoro-4-methoxy-phenyl)imidazo[1,2-a]pyrazin-8-yl]amino]benzoyl]piperazin-1-yl]-2-(methylamino)ethanone;
2-[5-chloro-4-[8-[3-chloro-4-[4-[3-(hydroxymethyl)piperazine-1-carbonyl]piperidine-1-carbonyl]anilino]imidazo[1,2-a]pyrazin-3-yl]-2-fluoro-phenoxy]acetonitrile;
[2-chloro-4-[[3-(3-fluoro-4-methoxy-phenyl)imidazo[1,2-a]pyrazin-8-yl]amino]phenyl]-[4-[2-(methylamino)ethyl]piperazin-1-yl]methanone;
N-(azetidin-3-ylmethyl)-1-[2-chloro-4-[[3-(3-fluoro-4-methoxy-phenyl)imidazo[1,2-a]pyrazin-8-yl]amino]benzoyl]piperidine-4-carboxamide;
[2-chloro-4-[[3-(3-fluoro-4-methoxy-phenyl)imidazo[1,2-a]pyrazin-8-yl]amino]phenyl]-[4-[2-(dimethylamino)ethyl]-4-hydroxy-1-piperidyl]methanone;
[2-chloro-4-[[3-(2,3-difluoro-4-methoxy-phenyl)imidazo[1,2-a]pyrazin-8-yl]amino]phenyl]-[4-[(2S,4R)-4-hydroxypyrrolidine-2-carbonyl]piperazin-1-yl]methanone;
2-[4-[8-[3-chloro-4-[4-[(2S,4R)-4-hydroxypyrrolidine-2-carbonyl]piperazine-1-carbonyl]anilino]imidazo[1,2-a]pyrazin-3-yl]-2,3-difluoro-phenoxy]acetonitrile; and
2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile.

In one embodiment, the present invention provides pharmaceutically acceptable salts of the compounds of formula (I) as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, N.Y. 2018). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The synthesis of the compound of formula (I) may, for example, be accomplished according to the general synthesis outlined in the following Scheme 1.

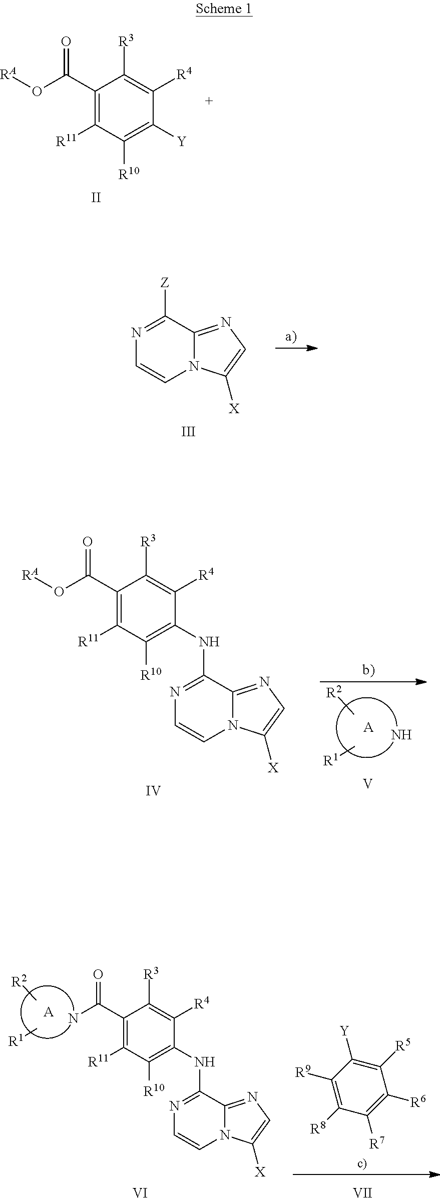

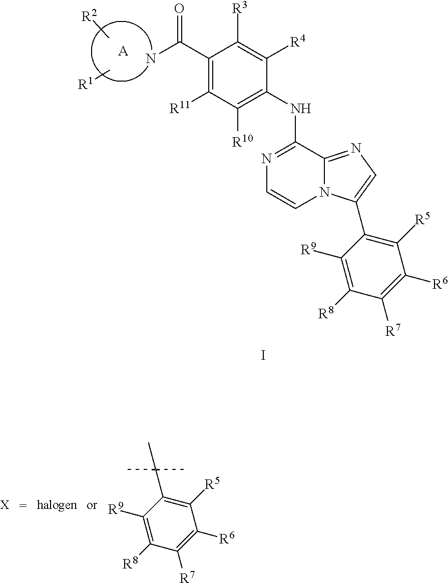

a) Acids or esters II, wherein Y is NH₂ or halogen and R^{A} is H or alkyl, are commercially available or can readily be accessed by methods known in the art and can conveniently be reacted with imidazopyrimidine derivatives III, which are likewise commercially available or can readily be accessed by methods known in the art, to access intermediates IV. Depending on the varying substitution (II: Y=NH₂ or halogen) it is convenient to react acids/esters II with the appropriate imidazopyrimidines derivative III (Z=NH₂ or halogen and X=halogen or appropriately substituted aryl moiety) under metal catalysis reaction conditions or nucleophilic aromatic substitution reaction conditions (as appropriate) to yield acids/esters IV.

b) Acid derivatives IV (R^{A}=H), can be accessed from esters IV (R^{A}=alkyl) upon saponification in the presence of a base. Examples of bases include: LiOH, NaOH and the like. Acid derivatives IV are conveniently reacted with an amine V, under varying coupling reaction conditions (coupling reaction conditions include: HATU, TBTU, and the like in the presence of a base, such as DIPEA, NEt₃, and the like) to afford amides VI. Amines V (and their protected congeners) are commercially available, known in the art or prepared according to methods known in the art. In case X=appropriately substituted aryl ring, these derivatives VI might be the final desired imidazopyridazines derivatives I, or any protecting group might have to be cleaved under appropriate conditions to afford final imidazopyridazines derivatives I. These imidazopyridazines I might be the final desired compounds, however might be further derivatised to yield final imidazopyridazines derivatives I.

c) Amides VI (X=halogen) are conveniently reacted under metal catalysis, such as PdCl₂(dppf)-CH₂Cl₂ adduct, Pd(PPh₃)₄, and the like and in the presence of a base, such as K₃PO₄, NaOtBu, and the like with the appropriate boronic acid or ester VII to afford imidazopyridazines derivatives I. These imidazopyridazines derivatives I might be the final desired compounds however any protecting group will have to be cleaved under appropriate conditions to afford final imidazopyridazines I. These imidazopyridazines I might be the final desired compounds, however might be further derivatised to yield final imidazopyridazines derivatives I.

In one aspect, the present invention provides a process of manufacturing the compounds of formula (I) described herein, comprising:
(i) reacting a carboxylic acid IVa, wherein R³ to R¹¹ are as defined herein,

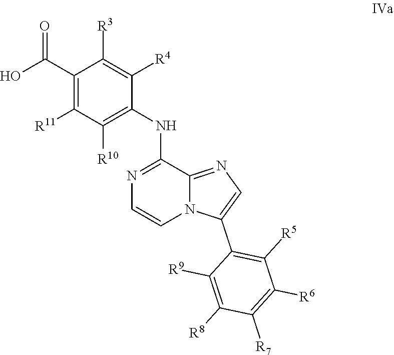

with an amine V, wherein A, R¹ and R² are as defined herein,

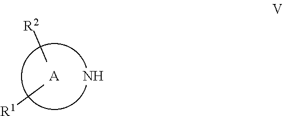

in the presence of a coupling reagent (such as HATU, TBTU, and the like) and a base (such as DIPEA, NEt₃, and the like), to form said compound of formula (I); or
(ii) reacting a compound VI, wherein R¹ to R⁴, R¹⁰, R¹¹ and A are as defined herein and X is halogen,

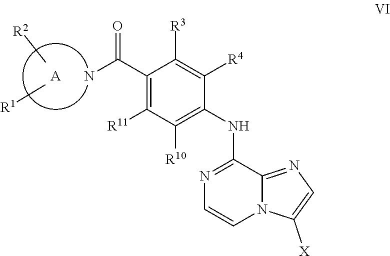

with a boronic acid VII, wherein R⁵ to R⁹ are as defined herein and Y is a boronic acid or a boronic acid ester,

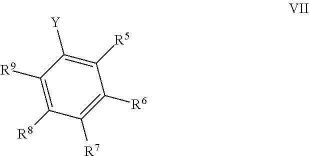

in the presence of a transition metal catalyst (such as PdCl₂(dppf)-CH₂Cl₂ adduct, Pd(PPh₃)₄, and the like) and a base (such as K₃PO₄, NaOtBu, and the like) to form said compound of formula (I).

In a further aspect, the present invention provides a compound of formula (I) as described herein, or a pharmaceutically acceptable salt thereof, when manufactured according to the processes disclosed herein.

### Using the Compounds of the Invention

As illustrated in the experimental section, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

The compounds of formula (I) and their pharmaceutically acceptable salts exhibit activity as antibiotics, particularly as antibiotics against Acinetobacter species, more particularly as antibiotics against Acinetobacter baumannii, most particularly as pathogen-specific antibiotics against Acinetobacter baumannii.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by Acinetobacter species, more particularly in the treatment and prevention of bacterial infections caused by Acinetobacter baumannii.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In one aspect, the present invention provides compounds of formula (I) or their pharmaceutically acceptable salts as described herein for use as therapeutically active substances.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a particular embodiment, said nosocomial infections and resulting diseases are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Gram-negative bacteria.

In a particular embodiment, said infections and resulting diseases caused by Gram-negative bacteria are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a particular embodiment, said infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, are selected from bacteremia, pneumonia, meningitis, urinary tract infection and wound infection, or a combination thereof.

In a further aspect, the present invention provides compounds of formula (I) or their pharmaceutically acceptable salts as defined above for use in the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides a method for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii, which method comprises administering a compound of formula (I) or a pharmaceutically acceptable salt thereof as defined above to a mammal.

In a further aspect, the present invention provides the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii. Such medicaments comprise compounds of formula (I) or their pharmaceutically acceptable salts as defined above.

### Pharmaceutical Compositions and Administration

In one aspect, the present invention provides pharmaceutical compositions comprising compounds of formula (I) or their pharmaceutically acceptable salts as defined above and one or more pharmaceutically acceptable excipients. Exemplary pharmaceutical compositions are described in Examples 115 to 118.

In a further aspect, the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

The following abbreviations are used in the present text:

(R)-BINAP=(R)-2,2′-bis(diphenylphosphino)-1,1′-binaphthyl, ACN=acetonitrile, aq.=aqueous, Boc=tert-butyloxycarbonyl, Boc-Glu-OtBu=Boc-L-glutamic acid 1-tert-butyl ester, Boc-Glu(OtBu)—OH=N-α-t.-Boc-L-glutamic acid γ-t.-butyl ester, Boc-Om(Z)—OH=Nα-Boc-Nδ-Cbz-L-ornithine, Nα-Boc-Nδ-Z-L-ornithine, Nδ-Z—Nα-Boc-L-ornithine, BrettPhos-Pd-G3=[(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate, CAS=chemical abstracts registration number, Cs₂CO₃=cesium carbonate, DCM=dichloromethane, DIAD=diisopropyl azodicarboxylate, DIPEA=ethyl diisopropylamine, DMA=N,N-dimethylacetamide, DMAP=4-(dimethylamino)-pyridine, DMF=N,N-dimethylformamide, DMSO=dimethylsulfoxide, DMSO-d6=deuterated dimethylsulfoxide, EA=ethyl acetate, EDC=1-ethyl-3-(3-dimethylaminopropyl)carbodiimide, EDCI=1-ethyl-3-(3-dimethylaminopropyl)carbodiimide, EI=electron impact, ESI=electrospray ionization, ESI⁺=electrospray ionization positive (mode), ESP=electrospray ionization positive (mode), Et₂O=diethylether, Et₃N=triethylamine, EtOAc=ethyl acetate, EtOH=ethanol, FA=formic acid, Fmoc-Agp(Boc)2-OH=N-α-Fmoc-N,N Æ-γ-di-t.-butoxycarbonyl-L-diaminobutanoic acid, Fmoc-Arg(Boc)2-OH=N-α-Fmoc-N-ω,N-ωÆ-bis-t-butoxycarbonyl-L-arginine, H₂=hydrogen, h=hour(s), HATU=1-[bis(dimethylamino)methylene]-1H-1,2,3-triazolo[4,5-b]pyridinium-3-oxide hexafluorophosphate, HCl=hydrochloric acid, HFIP=1,1,1,3,3,3-hexafluoroisopropanol, H₂O=water, HOBt=1-hydroxy-1H-benzotriazole, HPLC=high performance liquid chromatography, HV=high vacuum, ISN=ion spray negative (mode), K₂CO₃=potassium carbonate, KI=potassium iodide, KOH=potassium hydroxide, K₃PO₄=potassium phosphate tribasic, LC-MS=liquid chromatography coupled with mass spectroscopy, LiOH=lithium hydroxide, MeOH=methanol, MgSO₄=magnesium sulphate, min=minute(s), mL=milliliter, MS=mass spectrometry, MTBE=tert.-butyl methyl ether, N₂=nitrogen, Na₂CO₃=sodium carbonate, Na₂SO₃=sodium sulfite, Na₂SO₄=sodium sulfate, Na₂S₂O₃=sodium thiosulfate, NEt₃=triethylamine, NaHCO₃=sodium hydrogen carbonate, NaOH=sodium hydroxide, NH₄Cl=ammonium chloride, NiCl₂.6H₂O=nickel(II)chloride hexahydrate, NMO=N-methylmorpholine N-oxide, NMP=N-methyl-2-pyrrolidone, Pd/C=palladium on activated carbon, Pd₂(dba)₃=tris(dibenzylideneacetone)dipalladium(O), PdCl₂(PPh₃)₂=bis(triphenylphosphine)palladium(II) dichloride, Pd(dppf)Cl₂=[1,1′-bis(diphenylphosphino)ferrocene]dichloropalladium(II), PdCl₂(dppf)-CH₂Cl₂=[1,1′-bis(diphenylphosphino)ferrocene]dichloropalladium(II) dichloromethane complex, PE=petroleum ether, PhI(OAc)₂=(diacetoxyiodo)benzene, PPA=polyphosphoric acid, pTsOH=para toluenesulfonic acid, Rf=retention factor, RM=reaction mixture, RT=room temperature, SOCl₂=thionyl chloride, SFC=supercritical fluid chromatography, TBTU=2-(1H-Benzotriazole-1-yl)-1,1,3,3-tetramethylaminium tetrafluoroborate, T₃P=propylphosphonic anhydride, t-Bu-X-phos=2-di-tert-butylphosphino-2′,4′,6′-triisopropylbiphenyl, TEA=triethylamine, TEMPO=(2,2,6,6-tetramethylpiperidin-1-yl)oxyl, TFA=trifluoroacetic acid, THF=tetrahydrofurane, prep-TLC=preparative thin layer chromatography, UV=ultraviolet.

### Intermediate 1

### 4-((3-Iodoimidazo[1,2-a]pyrazin-8-yl)amino)-2-methylbenzoic Acid

A mixture of 8-chloro-3-iodoimidazo[1,2-a]pyrazine (100 mg, 358 μmol) and 4-amino-2-methylbenzoic acid (108 mg, 716 μmol) in 1,4-dioxane (2 mL) and acetic acid (2 mL) was stirred at 90° C. for 48 h. The mixture was allowed to cool to room temperature and filtered. The residue was washed with diethyl ether and dried in vacuo to give the title compound (139 mg) as a white solid. MS (ESI, m/z): 395.1 [M+H]⁺.

### Intermediate 2

### 2-Chloro-4-((3-(3-fluoro-4-methoxyphenyl)imidazo[1,2-a]pyrazin-8-yl)amino)benzoic acid

To 8-chloro-3-(3-fluoro-4-methoxyphenyl)imidazo[1,2-a]pyrazine (Intermediate 3, 50 mg, 180 μmol) in acetonitrile (0.9 mL) and acetic acid (100 μL) was added 4-amino-2-chlorobenzoic acid (46.3 mg, 270 μmol), followed by stirring at 80° C. overnight. The reaction mixture was filtered to give the title compound (70 mg) as a light brown solid. MS (ESI, m/z): 411.3 [M−H]⁻.

The following intermediates were prepared in analogy:

| | | MS ESI | |
|---|---|---|---|
| Int. | Name | [M + H]⁺ | Starting Material |
| 4 | 4-((3-(4-methoxyphenyl)imidazo[1,2-a]pyrazin- | 375.2 | 4-amino-2- |
| | 8-yl)amino)-2-methylbenzoic acid | | methylbenzoic acid |
| | | | and Intermediate 5 |
| 6 | 4-((3-(3-fluoro-4-methoxyphenyl)imidazo[1,2- | 393.3 | 4-amino-2- |
| | a]pyrazin-8-yl)amino)-2-methylbenzoic acid | | methylbenzoic acid |
| | | | and Intermediate 3 |
| 7 | 4-((3-(4-(difluoromethoxy)phenyl)imidazo[1,2- | 411 | 4-amino-2- |
| | a]pyrazin-8-yl)amino)-2-methylbenzoic acid | | methylbenzoic acid |
| | | | and Intermediate 8 |
| 9 | 2-chloro-4-[[3-(4-methoxyphenyl)imidazo[1,2- | 395.2 | 4-amino-2- |
| | a]pyrazin-8-yl]amino]benzoic acid | | chlorobenzoic acid |
| | | | and Intermediate 5 |
| 10 | 2-bromo-4-((3-(4- | 475.1 | 4-amino-2- |
| | (difluoromethoxy)phenyl)imidazo[1,2-a]pyrazin- | | bromobenzoic acid |
| | 8-yl)amino)benzoic acid | | and Intermediate 8 |
| 11 | 2-chloro-4-((3-(4- | 431.2 | 4-amino-2- |
| | (difluoromethoxy)phenyl)imidazo[1,2-a]pyrazin- | | chlorobenzoic acid |
| | 8-yl)amino)benzoic acid | | and Intermediate 8 |
| 12 | 4-((3-(4-(difluoromethoxy)phenyl)imidazo[1,2- | 425.1 | methyl 4-amino-2- |
| | a]pyrazin-8-yl)amino)-2-ethylbenzoic acid | | ethylbenzoate and |
| | | | Intermediate 13 |
| | | | followed by ester |
| | | | hydrolysis |
| 14 | 4-((3-(3-chloro-4-methoxyphenyl)imidazo[1,2- | 409.3 | methyl 4-amino-2- |
| | a]pyrazin-8-yl)amino)-2-methylbenzoic acid | | methylbenzoate and |
| | | | Intermediate 15 |
| | | | followed by ester |
| | | | hydrolysis |
| 16 | 4-((3-(3-chloro-4-methoxyphenyl)imidazo[1,2- | 395.1 | 4-aminobenzoic acid |
| | a]pyrazin-8-yl)amino)benzoic acid | | and Intermediate 15 |
| 17 | 4-((3-(4-methoxyphenyl)imidazo[1,2-a]pyrazin- | 360 | 4-aminobenzoic acid |
| | 8-yl)amino)benzoic acid | | and Intermediate 5 |
| 18 | 4-((3-(4-(difluoromethoxy)phenyl)imidazo[1,2- | 465.2 | 4-amino-2- |
| | a]pyrazin-8-yl)amino)-2-(trifluoromethyl)benzoic | | (trifluoromethyl)benzoic |
| | acid | | acid and Intermediate 8 |
| 19 | 4-((3-(4-(difluoromethoxy)phenyl)imidazo[1,2- | 442.2 | 4-amino-2- |
| | a]pyrazin-8-yl)amino)-2-nitrobenzoic acid | | nitrobenzoic acid and |
| | | | Intermediate 8 |
| 20 | 2-chloro-4-[[3-(2,3-difluoro-4-methoxy- | 431 | 4-amino-2- |
| | phenyl)imidazo[1,2-a]pyrazin-8- | | chlorobenzoic acid |
| | yl]amino]benzoic acid | | and Intermediate 21 |
| 22 | 2-chloro-4-[[3-[4-(difluoromethoxy)-2,3- | 467.1 | 4-amino-2- |
| | difluoro-phenyl]imidazo[1,2-a]pyrazin-8- | | chlorobenzoic acid |
| | yl]amino]benzoic acid | | and Intermediate 23 |
| 24 | 2-chloro-4-[[3-[2-chloro-4-(cyanomethoxy)-3- | 471.2 | 4-amino-2- |
| | fluoro-phenyl]imidazo[1,2-a]pyrazin-8- | | chlorobenzoic acid |
| | yl]amino]benzoic acid | | and Intermediate 25 |
| 26 | 4-[[3-(2-chloro-5-fluoro-4-methoxy- | 427.1 | methyl 4-amino-2- |
| | phenyl)imidazo[1,2-a]pyrazin-8-yl]amino]-2- | | methylbenzoate and |
| | methyl-benzoic acid | | Intermediate 27 |
| 28 | 4-[[3-(2,3-difluoro-4-methoxy- | 411.0 | 4-amino-2- |
| | phenyl)imidazo[1,2-a]pyrazin-8-yl]amino]-2- | | methylbenzoic acid |
| | methyl-benzoic acid | | and Intermediate 21 |
| 20 | 2-chloro-4-[[3-(2,3-difluoro-4-methoxy- | 431 | 4-amino-2- |
| | phenyl)imidazo[1,2-a]pyrazin-8- | | chlorobenzoic acid |
| | yl]amino]benzoic acid | | and Intermediate 21 |
| 29 | 2-chloro-4-[[3-[4-(cyanomethoxy)-2,3-difluoro- | 456.1 | 4-amino-2- |
| | phenyl]imidazo[1,2-a]pyrazin-8- | | chlorobenzoic acid |
| | yl]amino]benzoic acid | | and Intermediate 30 |
| 31 | 4-[[3-[4-(cyanomethoxy)-2,3-difluoro- | 436.1 | Intermediate 30 and |
| | phenyl]imidazo[1,2-a]pyrazin-8-yl]amino]-2- | | 4-amino-2- |
| | methyl-benzoic acid | | methylbenzoic acid |
| 32 | 4-[[3-[4-(cyanomethoxy)-2,3-difluoro- | 450.1 | Intermediate 33 and |
| | phenyl]imidazo[1,2-a]pyrazin-8-yl]amino]-2- | | Intermediate 30 |
| | ethyl-benzoic acid | | |
| 34 | 4-((3-(2-chloro-3-fluoro-4- | 427.2 | Intermediate 1 and 2- |
| | methoxyphenyl)imidazo[1,2-a]pyrazin-8- | | (2-chloro-3-fluoro-4- |
| | yl)amino)-2-methylbenzoic acid | | methox y-phenyl)- |
| | | | 4,4,5,5-tetramethyl- |
| | | | 1,3,2-dioxaborolane |
| 35 | 2-chloro-4-[[3-[3-chloro-4-(cyanomethoxy)-2- | 472.0 | Intermediate 36 and |
| | fluoro-phenyl]imidazo[1,2-a]pyrazin-8- | | 4-amino-2- |
| | yl]amino]benzoic acid | | chlorobenzoic acid |
| 37 | 2-chloro-4-[[3-[5-chloro-4-(cyanomethoxy)-2- | 471.9 | Intermediate 38 and |
| | fluoro-phenyl]imidazo[1,2-a]pyrazin-8- | | 4-amino-2- |
| | yl]amino]benzoic acid | | chlorobenzoic acid |
| 39 | 4-[[3-[5-chloro-4-(cyanomethoxy)-2-fluoro- | 452.1 | Intermediate 38 and |
| | phenyl]imidazo[1,2-a]pyrazin-8-yl]amino]-2- | | 4-amino-2- |
| | methyl-benzoic acid | | methylbenzoic acid |
| 40 | 4-[[3-[2-chloro-4-(cyanomethoxy)-3-fluoro- | 466.1 | Intermediate 41 and |
| | phenyl]imidazo[1,2-a]pyrazin-8-yl]amino]-2- | | Intermediate 33 |
| | ethyl-benzoic acid | | |
| 42 | methyl 2-ethyl-4-[(3-iodoimidazo[1,2-a]pyrazin- | 423.2 | 8-chloro-3- |
| | 8-yl)amino]benzoate | | iodoimidazo[1,2- |
| | | | a]pyrazine and |
| | | | methyl 4-amino-2- |
| | | | ethyl-benzoate (CAS |
| | | | No 1211589-24-0) |
| 43 | 4-((3-(4-(difluoromethoxy)-3- | 429.2 | Intermediate 44 and |
| | fluorophenyl)imidazo[1,2-a]pyrazin-8-yl)amino)- | | 4-amino-2- |
| | 2-methylbenzoic acid | | methylbenzoic acid |
| 45 | 2-cyano-4-((3-(4- | 422.2 | methyl 4-amino-2- |
| | (difluoromethoxy)phenyl)imidazo[1,2-a]pyrazin- | | cyanobenzoate |
| | 8-yl)amino)benzoic acid | | and Intermediate 8 |
| | | | followed by ester |
| | | | hydrolysis with LiOH |
| 46 | 4-((3-(4-(difluoromethoxy)phenyl)imidazo[1,2- | 523.1 | methyl 4-amino-2- |
| | a]pyrazin-8-yl)amino)-2-iodobenzoic acid | | iodobenzoate and |
| | | | Intermediate 8 |
| | | | followed by ester |
| | | | hydrolysis with LiOH |
| 47 | 4-((3-(4-(difluoromethoxy)phenyl)imidazo[1,2- | 423.1 | Intermediate 48 and |
| | a]pyrazin-8-yl)amino)-2-vinylbenzoic acid | | Intermediate 8 |
| | | | followed by ester |
| | | | hydrolysis with LiOH |
| 49 | methyl 4-((3-iodoimidazo[1,2-a]pyrazin-8- | 409.1 | From 8-chloro-3- |
| | yl)amino)-2-methylbenzoate | | iodoimidazo[1,2- |
| | | | a]pyrazine and |
| | | | methyl 4-amino-2- |
| | | | methylbenzoate |

### Intermediate 3

### 8-Chloro-3-(3-fluoro-4-methoxyphenyl)imidazo[1,2-a]pyrazine

To 8-chloro-3-iodoimidazo[1,2-a]pyrazine (500 mg, 1.79 mmol) in dioxane (6.5 mL) and water (3.25 mL) was added 2-(3-fluoro-4-methoxyphenyl)-4,4,5,5-tetramethyl-1,3,2-dioxaborolane (474 mg, 1.88 mmol), 1,1′-bis(diphenylphosphino)ferrocenedichloro palladium(II) dichloromethane complex (65.5 mg, 89.5 μmol) and sodium carbonate (379 mg, 3.58 mmol, Eq: 2) followed by stirring at 50° C. for 2 d. The reaction mixture was partitioned between ethyl acetate and water. The organic layers were dried over Na₂SO₄, filtered and concentrated to give a red solid, which was purified by column chromatography (silica gel, DCM/MeOH, 0-5%) to give the title compound (359 mg) as a pink-brown solid. MS (ESI, m/z): 278.1 [M+H]⁺.

The following intermediates were prepared in analogy to Intermediate 3:

| | | ESI MS | |
|---|---|---|---|
| Int. | Name | [M + H]⁺ | Starting Material |
| 5 | 8-Chloro-3-(4-methoxyphenyl)imidazo[1,2- | 260.1 | (4-methoxyphenyl) |
| | a]pyrazine | | boronic acid |
| 8 | 8-chloro-3-(4- | 296 | 2-(4- |
| | (difluoromethoxy)phenyl)imidazo[1,2-a]pyrazine | | (difluoromethoxy)phenyl)- |
| | | | 4,4,5,5-tetramethyl- |
| | | | 1,3,2-dioxaborolane |
| 50 | 4-(8-chloroimidazo[1,2-a]pyrazin-3-yl)phenol | 246.0 | 4- |
| | | | hydroxyphenylboronic |
| | | | acid |
| 13 | 8-chloro-3-(4-chloro-2,3- | 299.9 | (4-chloro-2,3- |
| | difluorophenyl)imidazo[1,2-a]pyrazine | | difluorophenyl)boronic |
| | | | acid |
| 15 | 8-chloro-3-(3-chloro-4- | 294.1 | (3-chloro-4- |
| | methoxyphenyl)imidazo[1,2-a]pyrazine | | methoxyphenyl)boronic |
| | | | acid |
| 51 | 8-chloro-3-(2-chloro-4-methoxy- | 293.1 | (2-chloro-4- |
| | phenyl)imidazo[1,2-a]pyrazine | | methoxyphenyl)boronic |
| | | | acid |
| 21 | 8-chloro-3-(2,3-difluoro-4-methoxy- | 296.0 | (2,3-difluoro-4- |
| | phenyl)imidazo[1,2-a]pyrazine | | methoxyphenyl)boronic |
| | | | acid |
| 30 | 2-[4-(8-chloroimidazo[1,2-a]pyrazin-3-yl)-2,3- | 321.0 | Intermediate 52 |
| | difluoro-phenoxy]acetonitrile | | |
| 36 | 2-[2-chloro-4-(8-chloroimidazo[1,2-a]pyrazin-3- | 337.0 | Intermediate 53 |
| | yl)-3-fluoro-phenoxy]acetonitrile | | |
| 38 | 2-[5-chloro-4-(8-chloroimidazo[1,2-a]pyrazin-3- | 337.0 | Intermediate 53 |
| | yl)-2-fluoro-phenoxy]acetonitrile | | |
| 44 | 8-chloro-3-[4-(difluoromethoxy)-3-fluoro- | 314.0 | 2-[4- |
| | phenyl]imidazo[1,2-a]pyrazine | | (difluoromethoxy)- |
| | | | 3-fluoro-phenyl]- |
| | | | 4,4,5,5-tetramethyl- |
| | | | 1,3,2-dioxaborolane |
| 25 | 2-[3-chloro-4-(8-chloroimidazo[1,2-a]pyrazin-3- | 339.0 | 2-[3-chloro-2- |
| | yl)-2-fluoro-phenoxy]acetonitrile | | fluoro-4-(4,4,5,5- |
| | | | tetramethyl-1,3,2- |
| | | | dioxaborolan-2- |
| | | | yl)phenoxy]acetonitrile |
| 54 | 4-(8-chloroimidazo[1,2-a]pyrazin-3-yl)-2,3- | 281.0 | (2,3-difluoro-4- |
| | difluoro-phenol | | hydroxyphenyl)boronic |
| | | | acid |

### Intermediate 52

### 2-[2,3-difluoro-4-(4,4,5,5-tetramethyl-1,3,2-dioxaborolan-2-yl)phenoxy]acetonitrile

### Step 1

### 2-(4-bromo-2,3-difluoro-phenoxy)acetonitrile

To a solution of 4-bromo-2,3-difluorophenol (5.2 g, 25 mmol, Eq: 1), bromoacetonitrile (6.0 g, 50 mmol, Eq: 2) in DMF (25 mL) was added potassium carbonate (6.9 g, 50 mmol, Eq: 2) and then the resultant mixture was stirred overnight at room temperature.

The mixture was poured into water (50 mL) and the aqueous solution was extracted with ethyl acetate (100 mL×2). The organic layers were combined and washed with water and brine, dried over anhydrous Na₂SO₄ and concentrated under reduced pressure. The residue was purified by prep. HPLC to give the title compound (5.2 g, 84% yield) as white solid.

MS (ESI, m/z): 248.0 [M+H]+.

### Step 2

### 2-[2,3-difluoro-4-(4,4,5,5-tetramethyl-1,3,2-dioxaborolan-2-yl)phenoxy]acetonitrile

To a solution of 2-(4-bromo-2,3-difluorophenoxy)acetonitrile (6.2 g, 25 mmol, Eq: 1) in dioxane (50 mL) and was added (4,4,4′,4′,5,5,5′,5′-octamethyl-2,2′-bi(1,3,2-dioxaborolane) (6.35 g, 25 mmol, Eq: 1), Pd(dppf)Cl2 (1.6 g, 2 mmol, Eq: 0.08) and potassium acetate (4.9 g, 50 mmol, Eq: 2) and then the resultant mixture was degassed for 5 min with nitrogen and then stirred overnight at 80° C. After cooling to room temperature, the mixture was poured into water (100 mL) and the aqueous solution was extracted with ethyl acetate (100 mL×2). The organic layers were combined and washed with water and brine, dried over anhydrous Na₂SO₄ and concentrated under reduced pressure to give a red oil which was purified by silica gel column chromatography to provide the desired compound (4 g, 54% yield) as an off-white solid.

### Intermediate 55

### 2-[3-chloro-2-fluoro-4-(4,4,5,5-tetramethyl-1,3,2-dioxaborolan-2-yl)phenoxy]acetonitrile

Prepared in analogy to Intermediate 52 starting from 4-bromo-3-chloro-2-fluoro-phenol [CAS #1360745-16-9].

### Intermediate 33

### 4-amino-2-ethyl-benzoic Acid

### Step 1: methyl 4-nitro-2-vinyl-benzoate & ethyl 4-nitro-2-vinyl-benzoate

A mixture of methyl 2-bromo-4-nitro-benzoate (5.2 g, 20 mmol, 1 eq), 2,4,6-trivinylcyclotriboroxane pyridine complex (5.78 g, 24 mmol, 1.2 eq), tetrakis(triphenylphosphine)palladium(0) (1.16 g, 1 mmol, 0.050 eq) and potassium carbonate (11.05 g, 79.99 mmol, 4 eq) in toluene (50 mL) and ethanol (50 mL) was stirred at 90° C. under nitrogen for 2 h. The mixture was filtered over Celite. The filtrate was concentrated to dryness. To the crude was added water (50 mL). The mixture was extracted with ethyl acetate (50 mL×3). The combined organic layers were concentrated to dryness. The crude was then purified by flash column chromatography eluting 10% ethyl acetate in petrol ether to afford methyl 4-nitro-2-vinyl-benzoate (1.58 g) as a brown oil.

### Step 2: ethyl 4-amino-2-ethyl-benzoate

A mixture of ethyl 4-nitro-2-vinyl-benzoate (392.0 mg, 1.77 mmol, 1 eq) and Pd/C (10%) (50.0 mg) in MeOH (10 mL) was stirred at 25° C. for 5 h under a hydrogen atmosphere. The mixture was filtered over Celite to afford ethyl 4-amino-2-ethyl-benzoate (331 mg, 1.71 mmol, 96.66% yield) as brown oil. MS (ESI⁺): 194.1 [(M+H)⁺].

### Step 3: 4-amino-2-ethyl-benzoic Acid

To a solution of methyl 4-amino-2-ethylbenzoate (540 mg, 3.0 mmol) in THF (5 mL) and methanol (25 mL) was added 2.0 M LiOH (3.0 mL) aqueous solution. The resultant mixture was stirred for 15 h at room temperature and then acidified to pH=5-6 with 3.0 M hydrochloric acid. The resulting suspension was filtered, the solid was washed with water and then dried to give the title compound (0.3 g, 60.5% yield) as a white solid

MS (ESI, m/z): 166.0 [M+H]+.

### Intermediate 56

### 2-(dimethylamino)-1-piperazin-1-yl-ethanone di-trifluoroacetate

### Step 1: tert-butyl 4-[2-(dimethylamino)acetyl]piperazine-1-carboxylate

To a solution of tert-butyl piperazine-1-carboxylate (500 mg, 2.68 mmol) in DMF (20 mL) was added dimethylglycine (277 mg, 2.68 mmol), triethylamine (815 mg, 1.12 mL, 8.05 mmol) and 1-propanephosphonic anhydride (1.71 g, 5.37 mmol), the reaction was stirred for 20 minutes at room temperature. The reaction mixture was quenched with water and washed with brine. The mixture was extracted in DCM. The organic layer was concentrated in vacuum to give crude product (530 mg), which was used in the next step without further purification. MS (ESI, m/z): [Ms+1]⁺ 272

### Step 2: 2-(dimethylamino)-1-piperazin-1-yl-ethanone di-trifluoroacetate

A solution of tert-butyl 4-(dimethylglycyl)piperazine-1-carboxylate (530 mg) in DCM (5 mL) and TFA (5 mL) was stirred for one hour at room temperature. The reaction mixture was concentrated in vacuo to give the crude product (680 mg), which was used without further purification. MS (ESI, m/z): [M+H]⁺ 172

### Intermediate 57

### 4-(3-aminopropyl)piperazin-2-one

### Step 1

### 2-[3-(3-oxopiperazin-1-yl)propyl]isoindoline-1,3-dione

A mixture of 3-(1,3-dioxoisoindolin-2-yl)propyl methanesulfonate (1.34 g, 5 mmol, Eq: 1), piperazin-2-one (600 mg, 6 mmol, Eq: 1.2) and potassium carbonate (1.38 g, 10 mmol, Eq: 2) in N,N-dimethylformamide (25 mL) was stirred at room temperature overnight. The mixture was diluted with H₂O and extracted with DCM. The DCM layer was dried and concentrated in vacuo to give a yellow oil, which was purified by flash column chromatography to provide the desired compound (1.2 g, 83.5% yield) as a white solid. MS (ESI, m/z): 278.1 [M+H]+.

### Step 2

### 4-(3-aminopropyl)piperazin-2-one

To a mixture of 2-(3-(3-oxopiperazin-1-yl)propyl)isoindoline-1,3-dione (1.15 g, 4 mmol) in EtOH (25 mL) was added hydrazine hydrate (2.0 mL) and then the mixture was stirred at room temperature overnight. The suspension was filtered and the filtrate was concentrated to give the title compound (0.5 g, 80% yield) as a yellow oil. MS (ESI, m/z): 158.1 [M+H]⁺.

The following intermediates were prepared in analogy to intermediate 57

| | | ESI MS | |
|---|---|---|---|
| Int. | Name | [M + H]⁺ | Starting Material |
| 58 | 4-(3-aminopropyl)-1-methyl- | 172.1 | 1-methylpiperazin- |
| | piperazin-2-one | | 2-one |
| 59 | tert-butyl 4-(3-aminopropyl)-3- | 258.1 | tert-butyl 3- |
| | oxo-piperazine-1-carboxylate | | oxopiperazine-1- |
| | | | carboxylate |
| 60 | 1-tert-butyl 2-methyl 4-(3- | 302.2 | 1-tert-butyl 2- |
| | aminopropyl)piperazine-1,2- | | methyl piperazine- |
| | dicarboxylate | | 1,2-dicarboxylate |
| 61 | 2-(2-imidazol-1- | 156.1 | Intermediate 62 and |
| | ylethoxy)ethanamine | | imidazole |

### Intermediate 63

### 2-ethyl-4-[(3-iodoimidazo[1,2-a]pyrazin-8-yl)amino]benzoic acid

### Step 1: methyl 2-ethyl-4-[(3-iodoimidazo[1,2-a]pyrazin-8-yl)amino]benzoate

To a solution of 8-chloro-3-iodo-imidazo[1,2-a]pyrazine (6.0 g, 21.47 mmol, 1 eq) in ACN (60 mL) was added methyl 4-amino-2-ethyl-benzoate [CAS #1211589-24-0] (4.72 g, 26.31 mmol, 1.23 eq) and acetic acid (6.0 mL, 21.47 mmol, 1 eq). The reaction mixture was stirred at 80° C. for 60 h. After cooling to room temperature, the reaction mixture was filtered and washed with (ACN:MeOH=10:1, V:V), and then dried to provide methyl 2-ethyl-4-[(3-iodoimidazo[1,2-a]pyrazin-8-yl)amino]benzoate (9.37 g, crude) as off-white solid. ¹H NMR (400 MHz, DMSO-d6) δ 10.14 (br s, 1H), 7.98-8.06 (m, 2H), 7.89 (s, 1H), 7.86 (d, J=4.77 Hz, 1H), 7.82 (d, J=8.53 Hz, 1H), 7.62 (d, J=4.77 Hz, 1H), 3.80 (s, 3H), 2.93 (q, J=7.40 Hz, 2H), 1.18 (t, J=7.40 Hz, 3H)

### Step 2: 2-ethyl-4-[(3-iodoimidazo[1,2-a]pyrazin-8-yl)amino]benzoic Acid

To a solution of methyl 2-ethyl-4-[(3-iodoimidazo[1,2-a]pyrazin-8-yl)amino]benzoate (9.37 g, 22.19 mmol, 1 eq) in THF (80 mL) was added sodium hydroxide (80.0 mL, 320 mmol, 14.42 eq) and then stirred at 60° C. for 60 h. The reaction mixture was adjusted to pH=1˜2 by 3N HCl, filtered and dried to 2-ethyl-4-[(3-iodoimidazo[1,2-a]pyrazin-8-yl)amino]benzoic acid (8.2 g, 20.09 mmol, 90.520% yield) as white solid. ¹H NMR (400 MHz, DMSO-d6) δ 12.48 (br s, 1H), 9.86 (s, 1H), 8.05 (dd, J=2.13, 8.66 Hz, 1H), 7.99 (d, J=2.01 Hz, 1H), 7.78-7.86 (m, 3H), 7.61 (d, J=4.64 Hz, 1H), 2.95 (q, J=7.40 Hz, 2H), 1.18 (t, J=7.47 Hz, 3H).

The following intermediates were prepared in analogy to Intermediate 63

| | | ESI MS | |
|---|---|---|---|
| Int. | Name | [M + H]⁺ | Starting Material |
| 64 | 2-chloro-4-[(3-iodoimidazo[1,2- | 415.1 | methyl 4-amino-2- |
| | a]pyrazin-8-yl)amino]benzoic acid | | chlorobenzoate |

### Reference Example 1

### 1-(4-((3-(4-(difluoromethoxy)phenyl)imidazo[1,2-a]pyrazin-8-yl)amino)-2-methylbenzoyl)piperidine-4-carboxylic Acid

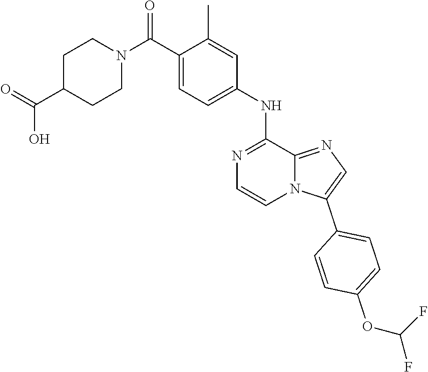 [SMILES: Cc1cc(Nc2nccn3c(-c4ccc(OC(F)F)cc4)cnc23)ccc1C(=O)N1CCC(C(=O)O)CC1]

### Step 1

### Methyl 1-(4-((3-(4-(difluoromethoxy)phenyl)imidazo[1,2-a]pyrazin-8-yl)amino)-2-methylbenzoyl)piperidine-4-carboxylate

A mixture of Intermediate 7, DIPEA (94.5 mg, 128 μL, 731 μmol) and HATU (185 mg, 487 μmol) in DMF (2 mL) was stirred for 30 min. Methyl piperidine-4-carboxylate (52.3 mg, 366 μmol) was added and stirring continued overnight. The mixture was purified by prep. HPLC to yield the title compound as a light brown solid (93 mg).

MS (ESI, m/z): 536.3 [M+H]⁺.

### Step 2

### 1-(4-((3-(4-(difluoromethoxy)phenyl)imidazo[1,2-a]pyrazin-8-yl)amino)-2-methylbenzoyl)piperidine-4-carboxylic Acid

A mixture of methyl 1-(4-((3-(4-(difluoromethoxy)phenyl)imidazo[1,2-a]pyrazin-8-yl)amino)-2-methylbenzoyl)piperidine-4-carboxylate (93 mg), 1M aq LiOH (0.8 mL) in THF (1 mL)/water (0.5 mL) was stirred at 60° C. for 5 h. The reaction mixture was concentrated and acidified by addition of 1M aq HCL. Water (1 mL) was added and the mixture was extracted with DCM. The combined organic layers were dried over sodium sulphate and then concentrated in vacuo to give the title compound (91 mg) as a white solid.

MS (ESI, m/z): 522.2 [M+H]⁺.

The following Examples and Intermediates were prepared in analogy to Reference Example 1

| | | | ESI MS | |
|---|---|---|---|---|
| Ex. | Name | Structure | [M + H]⁺ | Starting Material |
| REF 2 | 1-(4-((3-(3-fluoro-4- methoxyphenyl)imidazo [1,2-a]pyrazin-8- yl)amino)-2- methylbenzoyl)piperidine- 4-carboxylic acid | 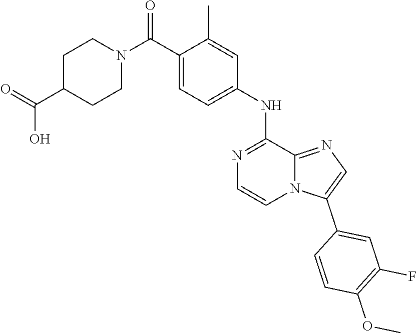 [SMILES: COc1ccc(-c2cnc3c(Nc4ccc(C(=O)N5CCC(C(=O)O)CC5)c(C)c4)nccn23)cc1F] | 504.2 | Intermediate 6 |
| 65 | 1-[4-[[3-(2,3-difluoro- 4-methoxy- phenyl)imidazo[1,2- a]pyrazin-8-yl]amino]- 2-methyl- benzoyl]piperidine-4- carboxylic acid | 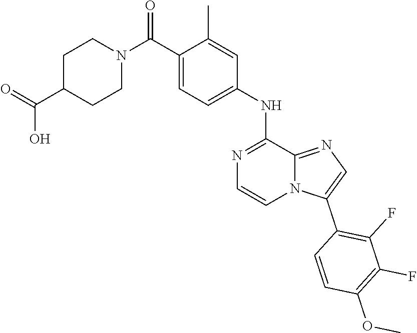 [SMILES: COc1ccc(-c2cnc3c(Nc4ccc(C(=O)N5CCC(C(=O)O)CC5)c(C)c4)nccn23)c(F)c1F] | 522.4 | Intermediate 28 |
| 66 | 1-(2-chloro-4-((3-(3- fluoro-4- methoxyphenyl)imidazo [1,2-a]pyrazin-8- yl)amino)benzoyl)piperidine- 4-carboxylic acid | 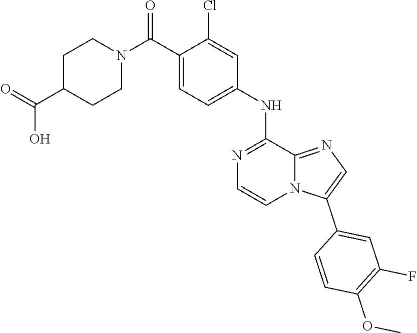 [SMILES: COc1ccc(-c2cnc3c(Nc4ccc(C(=O)N5CCC(C(=O)O)CC5)c(Cl)c4)nccn23)cc1F] | 524.3 | Intermediate 2 |
| 67 | 1-[2-chloro-4-[(3- iodoimidazo[1,2- a]pyrazin-8- yl)amino]benzoyl]piperidine- 4-carboxylic acid | 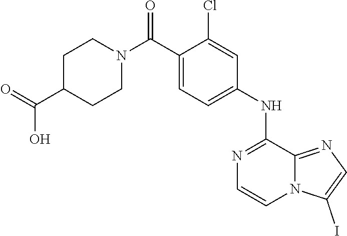 [SMILES: O=C(O)C1CCN(C(=O)c2ccc(Nc3nccn4c(I)cnc34)cc2Cl)CC1] | 526.3 | Intermediate 64 |
| 68 | 1-[4-[(3- iodoimidazo[1,2- a]pyrazin-8-yl)amino]- 2-methyl- benzoyl]piperidine-4- carboxylic acid | 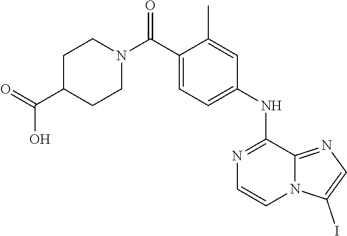 [SMILES: Cc1cc(Nc2nccn3c(I)cnc23)ccc1C(=O)N1CCC(C(=O)O)CC1] | 506.1 | Intermediate 1 |
| 69 | 1-(4-((3-(4- (cyanomethoxy)-2,3- difluorophenyl)imidazo [1,2-a]pyrazin-8- yl)amino)-2- methylbenzoyl)piperidine- 4-carboxylic acid | 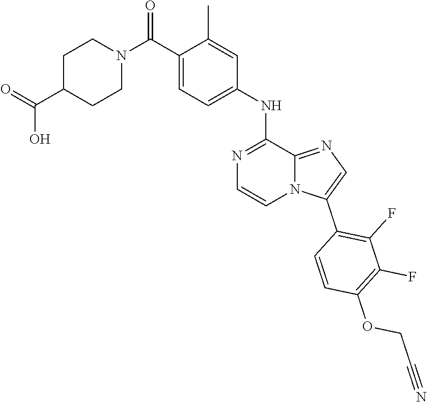 [SMILES: Cc1cc(Nc2nccn3c(-c4ccc(OCC#N)c(F)c4F)cnc23)ccc1C(=O)N1CCC(C(=O)O)CC1] | 547.3 | Intermediate 31 and piperidine-4- carboxylic acid (no hydrolysis step) |

### Reference Example 3

### 4-((3-(2,3-difluoro-4-methoxyphenyl)imidazo[1,2-a]pyrazin-8-yl)amino)-N,N,2-trimethylbenzamide

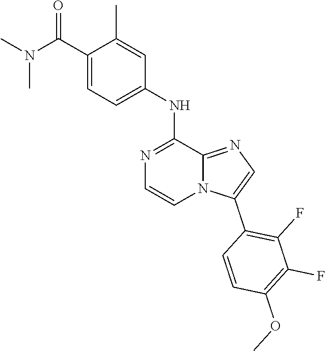 [SMILES: COc1ccc(-c2cnc3c(Nc4ccc(C(=O)N(C)C)c(C)c4)nccn23)c(F)c1F]

### Step 1

### 4-amino-N,N,2-trimethyl-benzamide

To a solution 4-amino-2-methylbenzoic acid (2.7 g, 18 mmol), dimethylamine hydrochloride (1.76 g, 21.6 mmol) in DCM (350 mL) was added TEA (3.6 g, 36 mmol) and then the resultant mixture was stirred for 30 min at room temperature, EDCI (4 g, 21 mmol) was added in the mixture and stirred for extra 10 h. The mixture was poured into water (500 mL) and the aqueous solution was extracted with DCM (100 mL×2). The organic layers were combined and washed with water and brine, dried over anhydrous sodium sulphate and concentrated under reduced pressure to give a yellow oil which was purified by flash column chromatography to provide the desired compound (2.5 g, 78% yield) as an off-white solid

MS (ESI, m/z): 179.1 [M+H]⁺.

### Step 2

### 4-((3-(2,3-difluoro-4-methoxyphenyl)imidazo[1,2-a]pyrazin-8-yl)amino)-N,N,2-trimethylbenzamide

To a solution of Intermediate 21 (295 mg, 1 mmol) in acetonitrile (10 mL) and acetic acid (1 mL) was added 4-amino-N,N,2-trimethyl-benzamide (178 mg, 1 mmol). The mixture was stirred overnight at 85° C. The mixture was poured into water (50 mL) and the aqueous solution was extracted with DCM (75 mL×2). The organic layers were combined and washed with water and brine, dried over anhydrous sodium sulphate and concentrated under reduced pressure to give a red oil which was purified by prep. HPLC to provide the desired compound (200 mg, 45.7% yield) as an off-white solid.

MS (ESI, m/z): 438.1 [M+H]⁺.

### Example 1

### 4-(2-chloro-4-((3-(2,3-difluoro-4-methoxyphenyl)imidazo[1,2-a]pyrazin-8-yl)amino)benzoyl)piperazine-2-carboxylic Acid

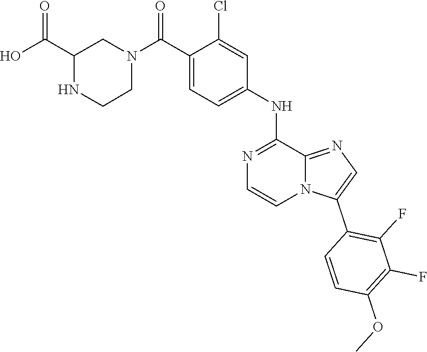 [SMILES: COc1ccc(-c2cnc3c(Nc4ccc(C(=O)N5CCNC(C(=O)O)C5)c(Cl)c4)nccn23)c(F)c1F]

### Step 1

### 1-tert-butyl 2-methyl 4-[2-chloro-4-[[3-(2,3-difluoro-4-methoxy-phenyl)imidazo[1,2-a]pyrazin-8-yl]amino]benzoyl]piperazine-1,2-dicarboxylate

To a solution of 2-chloro-4-((3-(2,3-difluoro-4-methoxyphenyl)imidazo[1,2-a]pyrazin-8-yl)amino)benzoic acid (215 mg, 0.5 mmol), 1-tert-butyl 2-methyl piperazine-1,2-dicarboxylate (146 mg, 0.6 mmol) in anhydrous DMF (5 mL) was added DIPEA (129 mg, 1.0 mmol) and then the resultant mixture was stirred for 30 min at room temperature, HATU (380 mg, 1.0 mmol) was added in the mixture and stirred for extra 10 h. The mixture was poured into water (50 mL) and the aqueous solution was extracted with ethyl acetate (50 mL×2). The organic layers were combined and washed with water and brine, dried and concentrated under reduced pressure to give a red oil, which was used in next step without purification. MS (ESI, m/z): 657.1 [M+H]⁺.

### Step 2

### Methyl 4-[2-chloro-4-[[3-(2,3-difluoro-4-methoxy-phenyl)imidazo[1,2-a]pyrazin-8-yl]amino]benzoyl]piperazine-2-carboxylate

To a solution of 1-tert-butyl 2-methyl 4-(2-chloro-4-((3-(2,3-difluoro-4-methoxyphenyl)imidazo[1,2-a]pyrazin-8-yl)amino)benzoyl)piperazine-1,2-dicarboxylate (200 mg, 0.3 mmol) in ethyl acetate (5 mL) was added 1 M hydrochloric acid in ethyl acetate (5.0 mL) at room temperature. The resultant mixture was stirred for 4 h and then adjusted to pH=7-8 with 2M aq. Na₂CO₃. The mixture was extracted with DCM (75 mL×2), the combined organic layers were washed with water and brine, dried and concentrated to give a red solid, which was used in the next step without purification.

MS (ESI, m/z): 557.1 [M+H]⁺.

### Step 3

### 4-[2-chloro-4-[[3-(2,3-difluoro-4-methoxy-phenyl)imidazo[1,2-a]pyrazin-8-yl]amino]benzoyl]piperazine-2-carboxylic Acid

To a solution of methyl 4-(2-chloro-4-((3-(2,3-difluoro-4-methoxyphenyl)imidazo[1,2-a]pyrazin-8-yl)amino)benzoyl)piperazine-2-carboxylate (167 mg, 0.3 mmol) in THF (5 mL) and MeOH ethyl acetate (5 mL) was added 1M aq. LiOH (3 mL) dropwise at room temperature. The resultant mixture was stirred for 4 h, and then acidified to pH 5-6 with 2 M hydrochloric acid. The mixture was extracted with DCM (50 mL×2), and the combined organic layers were washed with brine, and then dried and then concentrated to give a light yellow oil, which was purified by prep. HPLC to provide the desired compound (200 mg, 45.7% yield) as an off-white solid.

MS (ESI, m/z): 438.1 [M+H]⁺

The following Examples were prepared in analogy to Example 1

| | | | ESI MS | |
|---|---|---|---|---|
| Ex. | Name | Structure | [M + H]⁺ | Starting Material |
| REF 4 | 4-(3-(4- ((3-(3-fluoro- 4- methoxyphenyl) imidazo [1,2-a]pyrazin-8- yl)amino)-2- methyl- benzamido) propyl) piperazine-2- carboxylic acid | 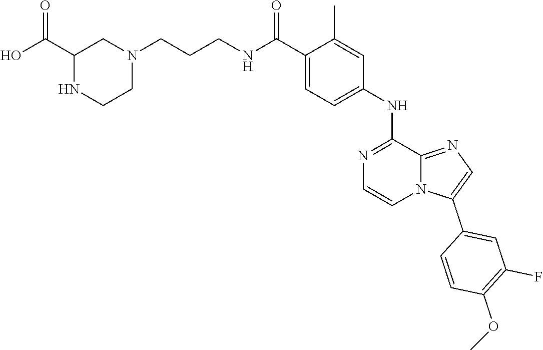 [SMILES: COc1ccc(-c2cnc3c(Nc4ccc(C(=O)NCCCN5CCNC(C(=O)O)C5)c(C)c4)nccn23)cc1F] | 562.2 | Intermediate 6 and Intermediate 60 |
| 2 | 4-(1-(2-chloro- 4-((3-(3- fluoro-4- methoxyphenyl) imidazo [1,2-a]pyrazin-8- yl)amino) benzoyl) piperidine-4- carbonyl) piperazine-2- carboxylic acid, formate salt | 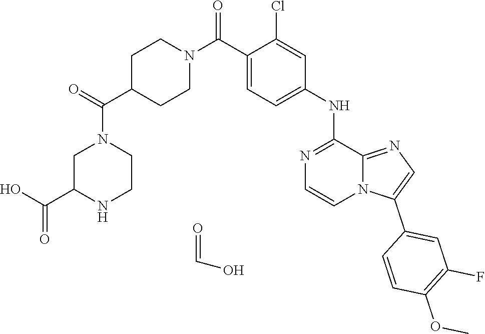 [SMILES: COc1ccc(-c2cnc3c(Nc4ccc(C(=O)N5CCC(C(=O)N6CCNC(C(=O)O)C6)CC5)c(Cl)c4)nccn23)cc1F ; O=CO] | 636.4 | Intermediate 66 and 1-(tert-butyl) 2- methyl piperazine- 1,2- dicarboxylate |

### Intermediate 23

### 8-chloro-3-[4-(difluoromethoxy)-2,3-difluoro-phenyl]imidazo[1,2-a]pyrazine

### Step 1: 1-bromo-4-(difluoromethoxy)-2,3-difluoro-benzene

A mixture of 4-bromo-2,3-difluorophenol (1 g, 4.78 mmol), sodium chlorodifluoroacetate (1.09 g, 7.18 mmol) and potassium carbonate (1.32 g, 9.57 mmol) in DMF (10 mL) was heated to 100° C. with stirring overnight. The mixture was diluted with saturated aq. NaHCO₃ solution and extracted with DCM. The DCM layer was dried and concentrated. The residue was purified by column chromatography (eluting with PE/EA=50/1) to give the title compound (1 g) as colorless oil.

### Step 2: 2-[4-(difluoromethoxy)-2,3-difluoro-phenyl]-4,4,5,5-tetramethyl-1,3,2-dioxaborolane

A mixture of 1-bromo-4-(difluoromethoxy)-2,3-difluorobenzene (850 mg), bis(pinacolato)diboron (833 mg, 3.28 mmol), potassium acetate (644 mg, 6.56 mmol) and PdCl₂(PPh₃)_{z}(115 mg, 164 μmol) in dioxane (20 mL) was heated to 100° C. with stirring overnight. The mixture was concentrated in vacuo and the residue was purified by column chromatography (eluting with PE/EA=30/1) to give the title compound (800 mg, 2.61 mmol) as colorless oil.

### Step 3: 8-chloro-3-[4-(difluoromethoxy)-2,3-difluoro-phenyl]imidazo[1,2-a]pyrazine

A mixture of 8-chloro-3-iodoimidazo[1,2-a]pyrazine (730 mg, 2.61 mmol), 2-(4-(difluoromethoxy)-2,3-difluorophenyl)-4,4,5,5-tetramethyl-1,3,2-dioxaborolane (800 mg, 2.61 mmol), PdCl₂(dppf)-CH₂Cl₂ adduct (95.6 mg, 131 μmol) and K₃PO₄ (1.66 g, 7.84 mmol) in THF (40 mL) and H₂O (10 mL) was heated to 50° C. with stirring overnight. The mixture was diluted with H₂O and extracted with DCM. The DCM layer was dried and concentrated. The residue was purified by silica gel column chromatography (eluting with PE/EA=5/1) to give the title compound (400 mg, 1.21 mmol) as a brown solid. MS (ESI, m/z): 332.2 [M+H]⁺

### Intermediate 41

### 2-[3-chloro-4-(8-chloroimidazo[1,2-a]pyrazin-3-yl)-2-fluoro-phenoxy]acetonitrile

### Step 1: 4-bromo-3-chloro-2-fluoro-phenol

To a stirred solution of 3-chloro-2-fluorophenol (10.00 g, 68.24 mmol) in DCM (200 mL) was added bromine (13.09 g, 81.88 mmol) dropwise at −10° C. The reaction mixture was warmed up to 20° C. and stirred for 16 h. The reaction was quenched with sat. aq. Na₂SO₃ (100 mL) and extracted with DCM (150 mL). The organic phase was washed with sat. NaHCO₃ (100 mL) and brine (100 mL), dried and concentrated under reduced pressure to give 4-bromo-3-chloro-2-fluoro-phenol (13.7 g) as a white solid. ¹H NMR (400 MHz, CDCl3) □: 7.31 (dd, 1H), 6.86 (t, 1H)

### Step 2: 2-(4-bromo-3-chloro-2-fluoro-phenoxy)acetonitrile

A mixture of 4-bromo-3-chloro-2-fluoro-phenol (13.70 g, 60.77 mmol), potassium carbonate (12.60 g, 91.16 mmol) and bromoacetonitrile (8.75 g, 72.92 mmol) in acetonitrile (200 mL) was stirred at 60° C. for 16 h. The reaction mixture was cooled and filtered. The filtrate was concentrated under reduced pressure, purified by the flash column chromatography (eluting with PE/EA=10/1) to give 2-(4-bromo-3-chloro-2-fluoro-phenoxy) acetonitrile (13.0 g) as a white solid. ¹H NMR (400 MHz, CDCl3) □: 7.43 (dd, 1H), 6.96 (dd, 1H), 4.84 (s, 2H)

### Step 3: 2-[3-chloro-2-fluoro-4-(4, 4, 5, 5-tetramethyl-1, 3, 2-dioxaborolan-2-yl)phenoxy]aceto Nitrile

A mixture of 2-(4-bromo-3-chloro-2-fluoro-phenoxy)acetonitrile (13.00 g, 49.15 mmol), bis(pinacolato)diboron (14.98 g, 58.98 mmol), potassium acetate (14.47 g, 147.46 mmol) and Pd(dppf)Cl₂.CH₂C₂ adduct (3.60 g, 4.92 mmol) in 1,4-dioxane (280 mL) was stirred at 70° C. under nitrogen for 16 h. The reaction was cooled and the mixture was filtered. The filtrate was concentrated under reduced pressure and the residue was purified by flash column chromatography (eluting with PE:EA=10:1) to afford desired product (11.6 g) as a light yellow solid.

### Step 4: 2-[3-chloro-4-(8-chloroimidazo[1,2-a]pyrazin-3-yl)-2-fluoro-phenoxy]acetonitrile

### Intermediate 41

A mixture of 8-chloro-3-iodo-imidazo[1,2-a]pyrazine (6.50 g, 23.26 mmol), 2-[3-chloro-2-fluoro-4-(4,4,5,5-tetramethyl-1,3,2-dioxaborolan-2-yl)phenoxy] acetonitrile (11.59 g, 23.26 mmol), sodium carbonate (7.40 g, 69.77 mmol) and Pd(dppf)Cl₂. CH₂Cl₂ adduct (1.7 g, 2.33 mmol) in 1,4-dioxane (150 mL) and water (30 mL) was stirred under nitrogen at 60° C. for 16 h. The reaction mixture was cooled and filtered. The filtrate was concentrated and the residue was diluted with H₂O (100 mL) and extracted with DCM (200 mL×3). The organic phase was washed with brine (100 mL), concentrated under reduced pressure and purified by flash column chromatography (PE/EA=1/1) to give crude product. It was re-purified by trituration (PE/EA=3/1) and dried in vacuo to afford the title compound (5.0 g) as a light red solid.

MS obsd. (ESI+) [(M+H)⁺]: 337.2

### Intermediate 27

### 8-chloro-3-(2-chloro-5-fluoro-4-methoxy-phenyl)imidazo[1,2-a]pyrazine

### Step 1: 1-chloro-4-fluoro-5-methoxy-2-nitro-benzene

Sodium (712 mg, 30.97 mmol) was added to MeOH (50 mL) and the mixture was stirred for 10 min. To the resulting mixture was added a solution of 1-chloro-4, 5-difluoro-2-nitro-benzene (5.0 g, 25.83 mmol) in MeOH (20 mL) at 0° C. The reaction was stirred at 15° C. for 2 h. The mixture was quenched with water (30 mL) and then extracted with DCM (100 mL). The DCM layer was washed with brine (30 mL), dried and concentrated under reduced pressure to give 1-chloro-4-fluoro-5-methoxy-2-nitro-benzene (4.2 g) as a yellow solid. ¹H NMR (400 MHz, CDCl3) □: 7.86 (d, 1H), 7.08 (d, 1H), 4.00 (s, 3H)

### Step 2: 2-chloro-5-fluoro-4-methoxy-aniline

To a stirred suspension of nickel(ii) chloride hexahydrate (2.44 g, 10.25 mmol) and sodium borohydride (380 mg, 10.04 mmol) in methanol (100 mL) was added a solution of 1-chloro-4-fluoro-5-methoxy-2-nitro-benzene (4.2 g, 20.43 mmol) in THF (40 mL) at 0° C. drop wise. Then additional sodium borohydride (2.31 g, 61.06 mmol) was added at 0° C. and the reaction mixture was stirred for 1 h at 15° C. The reaction was quenched by addition of water (20 mL). The solid was filtered and the filtrate was extracted with DCM. The organic phase was washed with brine, dried and concentrated under reduced pressure. The residue was purified by column chromatography (eluting with PE/EA=5/1) to give the title compound (2.8 g) as a yellow solid.

### Step 3: 1-bromo-2-chloro-5-fluoro-4-methoxy-benzene

To a solution of 2-chloro-5-fluoro-4-methoxy-aniline (1.7 g, 9.68 mmol) in aq. HBr (20 mL) was added sodium nitrite (735 mg, 10.65 mmol) in water (8 mL) at 0° C. The mixture was stirred at 0° C. for 30 min. Then a solution of copper (I) bromide (2.08 g, 14.52 mmol) and copper (II) bromide (3.24 g, 14.52 mmol) in aq. HBr (20 mL) was added to the mixture. The reaction was stirred at 60° C. for 2 h. The mixture was diluted with DCM (100 mL), washed with water (30 mL) and brine (30 mL), dried and concentrated under reduced pressure. The residue was purified by column chromatography (PE/EA=20/1) to give the title compound (530 mg) as a white solid.

### Step 4: 2-(2-chloro-5-fluoro-4-methoxy-phenyl)-4, 4, 5, 5-tetramethyl-1, 3, 2-dioxaborolane

A mixture of 1-bromo-2-chloro-5-fluoro-4-methoxy-benzene (530 mg, 2.21 mmol), bis(pinacolato)diboron (843 mg, 3.32 mmol), potassium acetate (652 mg, 6.64 mmol) and Pd(dppf)Cl₂.CH₂C₂ adduct (181 mg, 0.22 mmol) in 1,4-dioxane (2 mL) was stirred at 80° C. under nitrogen for 16 h. The reaction was cooled and the mixture was filtered. The filtrate was concentrated under reduced pressure and the residue was purified by flash column chromatography (PE/EA=100/1) to give desired compound (250 mg) as a white solid.

### Step 5: 8-chloro-3-(2-chloro-5-fluoro-4-methoxy-phenyl)imidazo[1,2-a]pyrazine

A mixture of intermediate 8-chloro-3-iodo-imidazo[1,2-a]pyrazine (400 mg, 1.43 mmol), 2-(2-chloro-5-fluoro-4-methoxy-phenyl)-4,4,5,5-tetramethyl-1,3,2-dioxaborolane (420 mg, 1.47 mmol), sodium carbonate (455 mg, 4.29 mmol) and Pd(dppf)Cl₂.CH₂C₂ adduct (117 mg, 0.14 mmol) in 1,4-dioxane (8 mL) and water (2 mL) was stirred under nitrogen at 50° C. for 16 h. The reaction mixture was cooled and filtered. The filtrate was concentrated under reduced pressure and the residue was purified by flash column chromatography (PE/EA=3/1) to give the desired product (375 mg) as a brown solid. MS obsd. (ESI⁺) [(M+H)+]: 312.2

### Intermediate 70

### 8-chloro-3-(2-chloro-3-fluoro-4-methoxy-phenyl)imidazo[1,2-a]pyrazine

### Step 1: 1-bromo-2-chloro-3-fluoro-4-methoxy-benzene

To a stirred solution of 1-chloro-2-fluoro-3-methoxy-benzene (2.00 g, 12.46 mmol) in chloroform (20 mL) was added bromine (1.89 g, 11.83 mmol) drop wise. The reaction mixture was stirred at 15° C. for 2 h. The reaction was quenched with aq. Na₂SO₃ solution and extracted with DCM. The organic phase was washed with brine (20 mL), dried over anhydrous sodium sulphate, concentrated under reduced pressure to give the desired compound (2.00 g) as a white solid.

### Step 2: 2-(2-chloro-3-fluoro-4-methoxy-phenyl)-4,4,5,5-tetramethyl-1,3,2-dioxaborolane

A mixture of 1-bromo-2-chloro-3-fluoro-4-methoxy-benzene (1.00 g, 2.8 mmol), bis(pinacolato)diboron (710 mg, 2.8 mmol), potassium acetate (824 mg, 8.39 mmol) and Pd (dppf)Cl₂.CH₂Cl₂ adduct (228 mg, 0.28 mmol) in 1,4-dioxane (20 mL) was stirred at 80° C. under nitrogen for 2 h. The reaction was cooled and the mixture was filtered. The filtrate was concentrated under reduced pressure and the residue was purified by flash column chromatography (eluting with PE/EA=50/1) to give the desired compound (200 mg) as a white solid.

### Step 3: 8-chloro-3-(2-chloro-3-fluoro-4-methoxy-phenyl)imidazo[1,2-a]pyrazine

A mixture of 2-(2-chloro-3-fluoro-4-methoxy-phenyl)-4,4,5,5-tetramethyl-1,3,2-dioxaborolane (195 mg, 0.68 mmol), 8-chloro-3-iodo-imidazo[1,2-a]pyrazine (190 mg, 0.68 mmol), sodium carbonate (216 mg, 2.04 mmol) and Pd(dppf)Cl₂.CH₂C₂ adduct (55 mg, 0.07 mmol) in 1,4-dioxane (4 mL) and water (1 mL) was stirred under nitrogen at 50° C. for 16 h. The reaction was cooled to RT and concentrated under reduced pressure. The residue was purified by prep-TLC (PE/EA=2/1) to afford desired compound (67 mg) as a white solid. MS obsd. (ESI⁺) [(M+H)⁺]: 312.

### Intermediate 71

### 2-[5-chloro-2-fluoro-4-(4,4,5,5-tetramethyl-1,3,2-dioxaborolan-2-yl)phenoxy]acetonitrile

### Step 1: 5-chloro-2-fluoro-4-nitro-phenol

A mixture of 1-chloro-4,5-difluoro-2-nitro-benzene (5.0 g, 25.83 mmol) and 15% aqueous KOH (2.9 g, 7.75 mmol) was stirred at 100° C. for 14 h. The mixture was added HCl (1N) until pH 4˜5 and extracted with DCM (100 mL×3). The mixture was then concentrated to dryness and purified by flash column chromatography (PE/EA=100%˜ 10%) to afford 5-chloro-2-fluoro-4-nitro-phenol (4.1 g, 21.41 mmol) as a yellow solid. MS obsd. (ESI⁻): 190.0 [(M−H)−].

### Step 2: 4-amino-5-chloro-2-fluoro-phenol

To a mixture of 5-chloro-2-fluoro-4-nitro-phenol (4.0 g, 20.88 mmol) and ammonium chloride (5.59 g, 104.42 mmol) in ethanol (60 mL) and water (30 mL) was added iron (5.83 g, 104.42 mmol). The mixture was stirred at 25° C. for 2 h. The mixture was filtered by celite. The filtrate was concentrated in vacuo to remove EtOH. The mixture was extracted with EA (30 mL×3). The combined organic layers were concentrated to dryness. The crude product was purified by flash column chromatography to (PE/EA=100% to 90%) afford 4-amino-5-chloro-2-fluoro-phenol (1.68 g) as a brown solid. MS obsd. (ESI⁺): 162.1 [(M−H)⁻].

### Step 3: 4-bromo-5-chloro-2-fluoro-phenol

To a mixture of 4-amino-5-chloro-2-fluoro-phenol (1.55 g, 9.57 mmol) in hydrobromic acid (19.69 mL, 145.02 mmol) was added a solution of sodium nitrite (0.79 g, 11.48 mmol) in water (8 mL) at 0° C. The mixture was kept at the same temperature for 30 min. Then a mixture of copper(II) bromide (0.67 mL, 14.35 mmol) and copper(I) bromide (2.06 g, 14.35 mmol) in hydrobromic acid (19.69 mL, 145.02 mmol) was added. The mixture was stirred at 60° C. for 14 h. The mixture was diluted with water (50 mL) and extracted with DCM (50 mL×3). The combined organic layers were concentrated to dryness. The crude was purified by flash column chromatography (PE/EA=100% to 90%) to afford 4-bromo-5-chloro-2-fluoro-phenol (1.89 g) as a white solid.

MS obsd. (ESI⁻): 223.0 [(M−H)⁻].

### Step 4: 2-(4-bromo-5-chloro-2-fluoro-phenoxy)acetonitrile

A mixture of 4-bromo-5-chloro-2-fluoro-phenol (1.89 g, 8.38 mmol) and potassium carbonate (3.48 g, 25.15 mmol) in acetone (150 mL) was stirred at 25° C. for 10 min. Then bromoacetonitrile (0.63 mL, 10.06 mmol) was added. The mixture was then stirred at 25° C. for 14 h. The mixture was concentrated to dryness and added water (20 mL). The mixture was extracted with ethyl acetate (10 mL×3). The combined organic layers were concentrated to dryness. The crude was purified by flash column chromatography (EA/PE=10%) to afford 2-(4-bromo-5-chloro-2-fluoro-phenoxy)acetonitrile (1.96 g) as a white solid. ¹H NMR (400 MHz, CDCl3) □: 7.44 (d, 1H), 7.23 (d, 1H), 4.83 (s, 2H)

### Step 5: 2-[5-chloro-2-fluoro-4-(4,4,5,5-tetramethyl-1,3,2-dioxaborolan-2-yl)phenoxy]acetonitrile

A mixture of 2-(4-bromo-5-chloro-2-fluoro-phenoxy)acetonitrile (1.96 g, 7.41 mmol), bis(pinacolato)diboron (2.26 g, 8.89 mmol), potassium acetate (1.39 mL, 22.23 mmol) and [1,1′-bis(diphenylphosphino)ferrocene]dichloropalladium(II) (542.26 mg, 0.740 mmol) in 1,4-dioxane (20 mL) was stirred at 100° C. under nitrogen for 14 h. The mixture was filtered over celite. The filtrate was concentrated to dryness. The crude product was then purified by flash column chromatography (EA/PE=5%) to afford 2-[5-chloro-2-fluoro-4-(4,4,5,5-tetramethyl-1,3,2-dioxaborolan-2-yl)phenoxy]acetonitrile (2.12 g) as a white solid.

¹H NMR (400 MHz, CDCl₃) δ ppm: 1.36 (s, 12H) 4.84 (s, 2H) 7.07 (d, J=7.0 Hz, 1H) 7.49 (d, J=11.3 Hz, 1H)

### Intermediate 72

### 2-(2-(2-hydroxyethoxy)ethyl)isoindoline-1,3-dione

A mixture of 2-(2-aminoethoxy)ethanol (3.51 g, 33.4 mmol) and isobenzofuran-1,3-dione (4.5 g, 30.4 mmol) in toluene (40 mL) was heated to 110° C. with stirring overnight. The mixture was concentrated in vacuo. The residue was diluted with water and extracted with DCM. The DCM layer was dried and concentrated to give crude 2-(2-(2-hydroxyethoxy)ethyl)isoindoline-1,3-dione (5.8 g, 81% yield) as yellow solid which was used in next step directly. MS obsd. (ESI⁺) [(M+H)⁺]: 236.

### Intermediate 62

### 2-[2-(1,3-dioxoisoindolin-2-yl)ethoxy]ethyl 4-methylbenzenesulfonate

To a solution of 2-(2-(2-hydroxyethoxy)ethyl)isoindoline-1,3-dione (2.5 g, 10.6 mmol) and TEA (2.15 g, 2.96 mL, 21.2 mmol) in DCM (40 mL) cooled at 0° C. was added 4-methylbenzene-1-sulfonyl chloride (4.05 g, 21.3 mmol). The mixture was warmed slowly to RT and stirred at RT overnight. The mixture was purified by column chromatography (eluting with PE/EA=2/1) to give 2-(2-(1,3-dioxoisoindolin-2-yl)ethoxy)ethyl 4-methylbenzenesulfonate (3.2 g, 78% yield) as white solid. MS obsd. (ESI⁺) [(M+H)⁺]: 390.

### Intermediate 73

### tert-butyl N-[[1-[4-[(3-iodoimidazo[1,2-a]pyrazin-8-yl)amino]-2-methyl-benzoyl]-4-piperidyl]methyl]carbamate

A mixture of 4-(Boc-aminomethyl)piperidine (1.77 g, 8.25 mmol), 4-((3-iodoimidazo[1,2-a]pyrazin-8-yl)amino)-2-methylbenzoic acid (Intermediate 1, 2.5 g, 6.34 mmol), HATU (3.62 g, 9.51 mmol) and Et₃N (1.93 g, 2.65 mL, 19 mmol) in DMF (30 mL) was stirred at room temperature overnight. The mixture was poured into water. The aqueous phase was extracted with DCM. The organic phase was washed with saturated NaCl solution and water. The organic phase was dried and concentrated in vacuo. The residue was purified by flash column to afford the title compound (3 g) as an orange oil. MS (ESI, m/z): 591 [M+H]+

The following intermediates were prepared in analogy:

| | | MS ESI | |
|---|---|---|---|
| Int. | Name | [M + H]⁺ | Starting Material |
| 74 | tert-butyl rac-(3R)-3-[[1-[2-chloro-4- | 694.4 | Intermediate 67 and |
| | [(3-iodoimidazo[1,2-a]pyrazin-8- | | tert-butyl (R)-3- |
| | yl)amino]benzoyl]piperidine-4- | | aminopyrrolidine-1- |
| | carbonyl]amino]pyrrolidine-1-carboxylate | | carboxylate |
| 75 | tert-butyl (2S,4R)-4-hydroxy-2-(4-(4-((3- | 676.2 | Intermediate 76 and |
| | iodoimidazo[1,2-a]pyrazin-8- | | (2S,4R)-1-(tert- |
| | yl)amino)-2-methylbenzoyl)piperazine-1- | | butoxycarbonyl)-4- |
| | carbonyl)pyrrolidine-1-carboxylate | | hydroxypyrrolidine- |
| | | | 2-carboxylic acid |

### Reference Example 5

### 2-chloro-4-[[3-(2,3-difluoro-4-methoxyphenyl)imidazo[1,2-a]pyrazin-8-yl]amino]-N-[2-(2-piperazin-1-ylethoxy)ethyl]benzamide;2,2,2-trifluoroacetic Acid

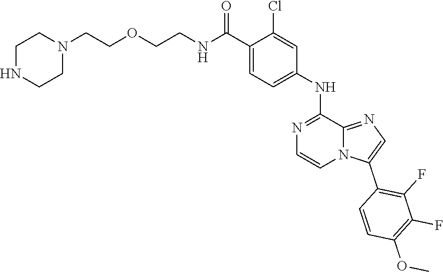 [SMILES: COc1ccc(-c2cnc3c(Nc4ccc(C(=O)NCCOCCN5CCNCC5)c(Cl)c4)nccn23)c(F)c1F]

### Step 1: tert-butyl 4-[2-[2-(1,3-dioxoisoindolin-2-yl)ethoxy]ethyl]piperazine-1-carboxylate

A mixture of Intermediate 62 (650 mg, 1.67 mmol), tert-butyl piperazine-1-carboxylate (466 mg, 2.5 mmol) and potassium carbonate (461 mg, 3.34 mmol) in DMF (10 mL) was stirred at RT overnight. The mixture was diluted with H₂O and extracted with DCM. The DCM layer was combined and washed with brine, concentrated and the residue was purified by column (silica gel, eluting with PE/EA=3/1) to give tert-butyl 4-(2-(2-(1,3-dioxoisoindolin-2-yl)ethoxy)ethyl)piperazine-1-carboxylate (700 mg) which was used in next step directly.

### Step 2: tert-butyl 4-[2-(2-aminoethoxy)ethyl]piperazine-1-carboxylate

To a solution of tert-butyl 4-(2-(2-(1,3-dioxoisoindolin-2-yl)ethoxy)ethyl)piperazine-1-carboxylate (700 mg, 1.73 mmol) in EtOH (10 mL) was added hydrazine hydrate (510 mg, 0.5 mL, 10.2 mmol). The mixture was stirred at rt overnight. The volatiles were removed and the residue was suspended in DCM and an insoluble solid was filtered off. The filtrate was concentrated in vacuo to give crude tert-butyl 4-(2-(2-aminoethoxy)ethyl)piperazine-1-carboxylate (500 mg) as light yellow oil which was used in next step directly.

### Step 3: tert-butyl 4-[2-[2-[[2-chloro-4-[[3-(2,3-difluoro-4-methoxy-phenyl)imidazo[1,2-a]pyrazin-8-yl]amino]benzoyl]amino]ethoxy]ethyl]piperazine-1-carboxylate

A mixture of tert-butyl 4-(2-(2-aminoethoxy)ethyl)piperazine-1-carboxylate (127 mg, 464 μmol), intermediate 20 (100 mg, 232 μmol), HATU (177 mg, 464 μmol) and TEA (363 mg, 0.5 mL) in DMF (5 mL) was stirred at rt overnight. The reaction was diluted with H₂O (50 mL) and extracted with DCM. The DCM layer was dried and concentrated in vacuo to give crude tert-butyl 4-(2-(2-(2-chloro-4-((3-(2,3-difluoro-4-methoxyphenyl)imidazo[1,2-a]pyrazin-8-yl)amino)benzamido)ethoxy)ethyl)piperazine-1-carboxylate (200 mg) as yellow oil which was used in the next step directly.

### Step 4: 2-chloro-4-[[3-(2,3-difluoro-4-methoxy-phenyl)imidazo[1,2-a]pyrazin-8-yl]amino]-N-[2-(2-piperazin-1-ylethoxy)ethyl]benzamide

To a solution of tert-butyl 4-(2-(2-(2-chloro-4-((3-(2,3-difluoro-4-methoxyphenyl)imidazo[1,2-a]pyrazin-8-yl)amino)benzamido)ethoxy)ethyl)piperazine-1-carboxylate (200 mg, 291 μmol) in MeOH (10 mL) was added TFA (2.96 g, 2 mL, 26 mmol). The mixture was heated to 50° C. with stirring overnight. The volatiles were removed and the residue was purified by prep-HPLC to give 2-chloro-4-((3-(2,3-difluoro-4-methoxyphenyl)imidazo[1,2-a]pyrazin-8-yl)amino)-N-(2-(2-(piperazin-1-yl)ethoxy)ethyl)benzamide (30 mg) as light yellow solid. MS obsd. (ESI+) [(M+H)+]. 586.

### Reference Example 6

### 2-chloro-4-[[3-(2,3-difluoro-4-methoxy-phenyl)imidazo[1,2-a]pyrazin-8-yl]amino]-N-[2-[2-(dimethylamino)ethoxy]ethyl]benzamide;2,2,2-trifluoroacetic Acid

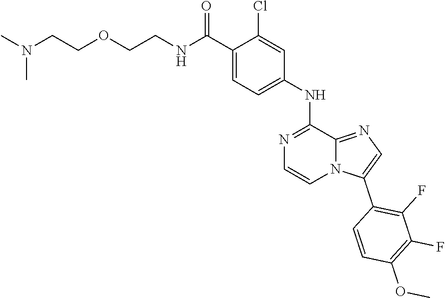 [SMILES: COc1ccc(-c2cnc3c(Nc4ccc(C(=O)NCCOCCN(C)C)c(Cl)c4)nccn23)c(F)c1F]

### Step 1: 2-[2-[2-(dimethylamino)ethoxy]ethyl]isoindoline-1,3-dione

A mixture of Intermediate 62 (400 mg, 1.03 mmol), dimethylamine (770 μL, 1.54 mmol) and potassium carbonate (284 mg, 2.05 mmol) in acetonitrile (10 mL) was stirred at RT overnight. The mixture was diluted with H₂O and extracted with DCM. The DCM layer was combined and washed with brine, concentrated to give crude 2-(2-(2-(dimethylamino)ethoxy)ethyl)isoindoline-1,3-dione (300 mg) which was used in next step directly.

### Step 2: 2-[2-(dimethylamino)ethoxy]ethanamine

To a solution of 2-(2-(2-(dimethylamino)ethoxy)ethyl)isoindoline-1,3-dione (300 mg, 1.14 mmol) in EtOH (10 mL) was added hydrazine hydrate (57.3 mg, 1.14 mmol). The reaction was stirred at RT overnight. The solid was filtered and the filtrate was concentrated in vacuo to give 2-(2-aminoethoxy)-N,N-dimethylethanamine (150 mg) as light yellow oil which was used in next step directly.

### Step 3: 2-chloro-4-[[3-(2,3-difluoro-4-methoxy-phenyl)imidazo[1,2-a]pyrazin-8-yl]amino]-N-[2-[2-(dimethylamino)ethoxy]ethyl]benzamide

A mixture of Intermediate 20 (100 mg, 232 μmol), 3-(2-(dimethylamino)ethoxy)propan-1-amine (33.9 mg, 232 μmol), HATU (177 mg, 464 μmol) and TEA (363 mg, 0.5 mL, 3.59 mmol) in DMF (5 mL) was stirred at rt overnight. The mixture was diluted with H₂O (50 mL) and extracted with DCM (50 mL) for three times. The DCM layer was dried and concentrated in vacuo. The residue was purified by prep-HPLC to give the title compound (40 mg) as light yellow solid. MS (ESI+) [M+H]⁺: 545.1.

### Reference Example 7

### 4-[[3-(2,3-difluoro-4-methoxy-phenyl)imidazo[1,2-a]pyrazin-8-yl]amino]-2-methyl-N-[2-[2-(3-oxopiperazin-1-yl)ethoxy]ethyl]benzamide;2,2,2-trifluoroacetic acid

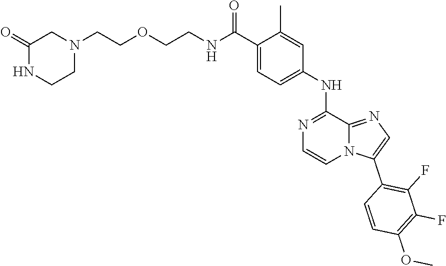 [SMILES: COc1ccc(-c2cnc3c(Nc4ccc(C(=O)NCCOCCN5CCNC(=O)C5)c(C)c4)nccn23)c(F)c1F]

### Step 1: 2-[2-(1,3-dioxoisoindolin-2-yl)ethoxy]ethyl methanesulfonate

To a solution of Intermediate 72 (2 g, 8.5 mmol) and TEA (1.45 g, 2 mL) in CH₂Cl₂ (50 mL) cooled at 0° C. was added MsCl (1.07 g, 9.35 mmol). The mixture was warmed to RT and stirred at RT for 4 h. The mixture was concentrated in vacuo and the residue was purified by column chromatography (eluting with PE/EA=1/1) to give 2-(2-(1,3-dioxoisoindolin-2-yl)ethoxy)ethyl methanesulfonate

### Step 2: 2-[2-[2-(3-oxopiperazin-1-yl)ethoxy]ethyl]isoindoline-1,3-dione

A mixture of 2-(2-(1,3-dioxoisoindolin-2-yl)ethoxy)ethyl methanesulfonate (500 mg, 1.6 mmol), piperazin-2-one (192 mg, 1.91 mmol) and K₂CO₃ (441 mg, 3.19 mmol) in DMF (5 mL) was heated to 100° C. with stirring overnight. The mixture was diluted with H₂O and extracted with DCM. The DCM layer was dried and concentrated in vacuo to give crude 2-(2-(2-(3-oxopiperazin-1-yl)ethoxy)ethyl)isoindoline-1,3-dione (550 mg) as yellow oil which was used in next step directly.

### Step 3: 4-[2-(2-aminoethoxy)ethyl]piperazin-2-one

A mixture of crude 2-(2-(2-(3-oxopiperazin-1-yl)ethoxy)ethyl)isoindoline-1,3-dione (550 mg, 1.73 mmol, Eq: 1) and hydrazine monohydrate (104 mg, 2.08 mmol) in EtOH (5 mL) was stirred at RT overnight. The mixture was concentrated in vacuo and the solid residue was suspended in DCM. The mixture was stirred at RT for 30 min and filtered. The filtrate was concentrated in vacuo to give crude 4-(2-(2-aminoethoxy)ethyl)piperazin-2-one (350 mg) as a yellow oil which was used in next step directly.

### Step 4: 4-[[3-(2,3-difluoro-4-methoxy-phenyl)imidazo[1,2-a]pyrazin-8-yl]amino]-2-methyl-N-[2-[2-(3-oxopiperazin-1-yl)ethoxy]ethyl]benzamide

A mixture of Intermediate 28 (150 mg, 366 μmol), 4-(2-(2-aminoethoxy)ethyl)piperazin-2-one (137 mg, 731 μmol), TEA (363 mg, 0.5 mL) and HATU (278 mg, 731 μmol) in DMF (5 mL) was stirred at rt. The mixture was diluted with H₂O (30 mL) and extracted with ethyl acetate. The ethyl acetate layer was concentrated and the residue was purified by prep-HPLC to give the title compound (36 mg) as light yellow solid. (ESI+) [(M+H)⁺]: 580.

### Reference Example 8

### 4-[[3-(3-fluoro-4-methoxy-phenyl)imidazo[1,2-a]pyrazin-8-yl]amino]-N,2-dimethyl-N-(3-oxo-3-piperazin-1-yl-propyl)benzamide

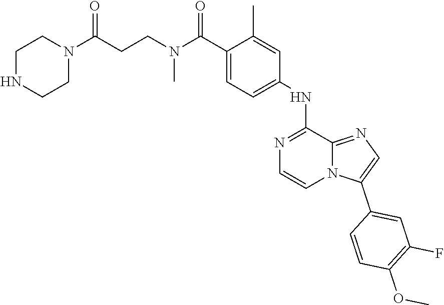 [SMILES: COc1ccc(-c2cnc3c(Nc4ccc(C(=O)N(C)CCC(=O)N5CCNCC5)c(C)c4)nccn23)cc1F]

### Step 1: ethyl 3-[[4-[[3-(3-fluoro-4-methoxy-phenyl)imidazo[1,2-a]pyrazin-8-yl]amino]-2-methyl-benzoyl]-methyl-amino]propanoate

To a stirred solution of ethyl 3-(methylamino) propanoate (100 mg, 0.76 mmol), Intermediate 6 (200 mg, 0.51 mmol) and triethylamine (0.2 mL, 1.53 mmol) in DMF (3 mL) was added 1-propanephosphonic anhydride (487 mg, 0.76 mmol, 50% in ethyl acetate) slowly. The reaction was stirred at 15° C. for 4 h. The reaction mixture was diluted with H₂O (10 mL) and extracted with ethyl acetate. The organic phase was washed with brine, dried over anhydrous sodium sulphate and concentrated under reduced pressure to give the title compound (250 mg) as a yellow oil. MS (ESI, m/z): 506 [M+H]⁺.

### Step 2: 3-[[4-[[3-(3-fluoro-4-methoxy-phenyl)imidazo[1,2-a]pyrazin-8-yl]amino]-2-methyl-benzoyl]-methyl-amino]propanoic Acid

To a stirred solution of ethyl 3-[[4-[[3-(3-fluoro-4-methoxy-phenyl)imidazo[1,2-a]pyrazin-8-yl]amino]-2-methyl-benzoyl]-methyl-amino]propanoate (250 mg, 0.49 mmol) in ethanol (3 mL) was added a solution of sodium hydroxide (40 mg, 0.99 mmol) in water (0.5 mL) slowly. The reaction was stirred at 30° C. for 4 h. Aq. HCl (1.0 M) was added drop wise until pH=4-5. The reaction mixture was concentrated under reduced pressure and the residue was purified by prep-HPLC to afford the title compound (59.4 mg) as a white solid. MS obsd. (ESI⁺) [(M+H)⁺]: 478

### Step 3: tert-butyl 4-[3-[[4-[[3-(3-fluoro-4-methoxy-phenyl)imidazo[1,2-a]pyrazin-8-yl]amino]-2-methyl-benzoyl]-methyl-amino]propanoyl]piperazine-1-carboxylate

To a stirred mixture of 3-[[4-[[3-(3-fluoro-4-methoxy-phenyl)imidazo[1,2-a]pyrazin-8-yl]amino]-2-methyl-benzoyl]-methyl-amino]propanoic acid (140 mg, 0.29 mmol), Boc-piperazine hydrochloride (98 mg, 0.44 mmol) and triethylamine (0.12 mL, 0.88 mmol) in DMF (2 mL) was added 1-propanephosphonic anhydride (280 mg, 0.44 mmol, 50% in ethyl acetate) slowly at 15° C. The reaction was stirred for 4 h. The reaction mixture was diluted with H₂O (10 mL) and extracted with ethyl acetate. The organic phase was washed with brine (10 mL), dried and concentrated under reduced pressure to give the title compound (170 mg) as a yellow oil. MS obsd. (ESI⁺) [(M+H)⁺]: 646

### Step 4: 4-[[3-(3-fluoro-4-methoxy-phenyl)imidazo[1,2-a]pyrazin-8-yl]amino]-N,2-dimethyl-N-(3-oxo-3-piperazin-1-yl-propyl)benzamide

A mixture of tert-butyl 4-[3-[[4-[[3-(3-fluoro-4-methoxy-phenyl)imidazo[1,2-a]pyrazin-8-yl]amino]-2-methyl-benzoyl]-methyl-amino]propanoyl]piperazine-1-carboxylate (170 mg, 0.26 mmol) and a solution of HCl in MeOH (0.2 mL, 0.79 mmol) in methanol (2 mL) was stirred at 15° C. for 4 h. The reaction mixture was concentrated under reduced pressure and purified by prep-HPLC to give the title compound (7.7 mg) as a white solid. MS obsd. (ESI+) [(M+H)+]: 546.1.

### Reference Example 9

### 4-[[3-(3-fluoro-4-methoxy-phenyl)imidazo[1,2-a]pyrazin-8-yl]amino]-N,2-dimethyl-N-(4-oxo-4-piperazin-1-yl-butyl)benzamide

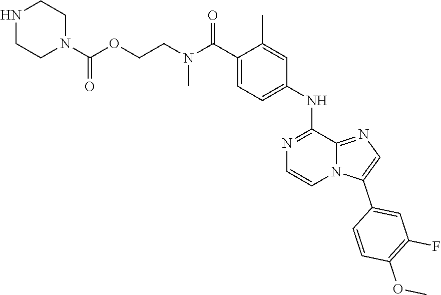 [SMILES: COc1ccc(-c2cnc3c(Nc4ccc(C(=O)N(C)CCOC(=O)N5CCNCC5)c(C)c4)nccn23)cc1F]

Reference Example 9 was prepared using same procedure as for Reference Example 8, changing ethyl 3-(methylamino) propanoate to methyl 4-(methylamino) butanoate hydrochloride. The title compound was purified by prep-HPLC. MS (ESI, m/z): 560.1 [M+H]⁺.

### Intermediate 77

### 2-[[4-[[3-(3-fluoro-4-methoxy-phenyl)imidazo[1,2-a]pyrazin-8-yl]amino]-2-methyl-benzoyl]-methyl-amino]acetic Acid

### Step 1: methyl 2-[methyl-(2-methyl-4-nitro-benzoyl)amino]acetate

A mixture of 2-methyl-4-nitro-benzoic acid (2.00 g, 11.04 mmol), EDC hydrochloride (3.17 g, 16.56 mmol), HOBt (2.24 g, 16.56 mmol) and DIPEA (5.77 mL, 33.12 mmol) in DMF (40 mL) was stirred at 15° C. for 0.5 h. Then sarcosine methyl ester hydrochloride (2.31 g, 16.56 mmol) was added and the mixture was stirred at 15° C. for 16 h. The reaction mixture was diluted with H₂O (50 mL) and extracted with ethyl acetate. The organic phase was washed with brine, dried, concentrated under reduced pressure and purified by flash column chromatography (eluting with PE:EA=3:1) to afford the title compound (1.00 g) as brown oil. MS obsd. (ESI⁺) [M+H]⁺: 267

### Step 2: methyl 2-[(4-amino-2-methyl-benzoyl)-methyl-amino]acetate

A mixture of methyl 2-[methyl-(2-methyl-4-nitro-benzoyl)amino]acetate (1.00 g, 3.76 mmol) and palladium (200 mg, 1.88 mmol, 10 wt % on charcoal) in methanol (20 mL) was stirred under hydrogen (15 psi) at 15° C. for 16 h. The mixture was filtered and the filtrate was concentrated under reduced pressure to give the title compound (850 mg) as a crude product which was used directly in the next step.

### Step 3: methyl 2-[[4-[[3-(3-fluoro-4-methoxy-phenyl)imidazo[1,2-a]pyrazin-8-yl]amino]-2-methyl-benzoyl]-methyl-amino]acetate

A mixture of Intermediate 3 (950 mg, 3.42 mmol) and methyl 2-[[4-[[3-(3-fluoro-4-methoxy-phenyl)imidazo[1,2-a]pyrazin-8-yl]amino]-2-methyl-benzoyl]-methyl-amino]acetate (808 mg, 3.42 mmol) in acetonitrile (18 mL) and acetic acid (2 mL) was stirred at 100° C. for 4 h. The reaction was cooled and concentrated under reduced pressure. The residue was purified by flash column chromatography (eluting with DCM/MeOH=50/1) to afford the title compound (1.20 g) as a yellow solid. MS obsd. (ESI⁺) [(M+H)⁺]: 478

### Step 4: 2-[[4-[[3-(3-fluoro-4-methoxy-phenyl)imidazo[1,2-a]pyrazin-8-yl]amino]-2-methyl-benzoyl]-methyl-amino]acetic Acid

Into a stirred solution of methyl 2-[[4-[[3-(3-fluoro-4-methoxy-phenyl)imidazo[1,2-a]pyrazin-8-yl]amino]-2-methyl-benzoyl]-methyl-amino]acetate (1.10 g, 2.3 mmol) in methanol (20 mL) was added a solution of sodium hydroxide (276 mg, 6.91 mmol) in water (3.5 mL). The reaction was stirred at 30° C. for 4 h and then cooled and concentrated. The residue was diluted with H₂O (20 mL) and acidified with aq. HCl (1.0 M) until pH=5-6. The precipitate was collected by filtration and then triturated (acetonitrile) to afford the title compound (1.02 g) as a white solid, which was used without further purification in the subsequent steps. (ESI⁺) [(M+H)⁺]: 464.1

### Reference Example 10

### 2-[[4-[[3-(3-fluoro-4-methoxy-phenyl)imidazo[1,2-a]pyrazin-8-yl]amino]-2-methyl-phenyl]methyl-methyl-amino]-1-piperazin-1-yl-ethanone Hydrochloride

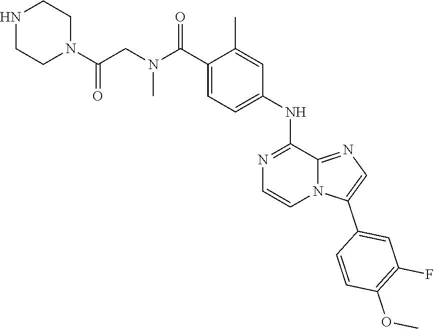 [SMILES: COc1ccc(-c2cnc3c(Nc4ccc(C(=O)N(C)CC(=O)N5CCNCC5)c(C)c4)nccn23)cc1F]

### Step 1: tert-butyl 4-[2-[[4-[[3-(3-fluoro-4-methoxy-phenyl)imidazo[1,2-a]pyrazin-8-yl]amino]-2-methyl-benzoyl]-methyl-amino]acetyl]piperazine-1-carboxylate

Into a stirred solution of Intermediate 77 (206. mg, 0.44 mmol), Boc-piperazine hydrochloride (119 mg, 0.53 mmol) and triethylamine (0.19 mL, 1.33 mmol) in DMF (3 mL) was added 1-propanephosphonic anhydride (425 mg, 0.67 mmol, 50% in ethyl acetate) slowly. The reaction was stirred at 15° C. for 4 h. The reaction mixture was diluted with H₂O (10 mL) and extracted with ethyl acetate. The organic phase was washed with brine, dried over anhydrous sodium sulphate and concentrated under reduced pressure to give the title compound (300 mg) as a yellow oil which was used directly in the next step. MS obsd. (ESI⁺) [(M+H)⁺]: 632

### Step 2: 2-[[4-[[3-(3-fluoro-4-methoxy-phenyl)imidazo[1,2-a]pyrazin-8-yl]amino]-2-methyl-phenyl]methyl-methyl-amino]-1-piperazin-1-yl-ethanone hydrochloride

A mixture of tert-butyl 4-[2-[[4-[[3-(3-fluoro-4-methoxy-phenyl)imidazo[1,2-a]pyrazin-8-yl]amino]-2-methyl-benzoyl]-methyl-amino]acetyl]piperazine-1-carboxylate (200 mg, 0.32 mmol) and a solution of HCl in 1,4-dioxane (0.4 mL, 1.58 mmol) in methanol (2 mL) was stirred at 15° C. for 4 h. The reaction mixture was concentrated under reduced pressure and the residue was purified by prep-HPLC to afford the title compound (88 mg) as a white solid. MS obsd. (ESI⁺) [(M+H)⁺]: 532

The following intermediates were prepared in analogy:

| | | | MS | |
|---|---|---|---|---|
| | | | ESI | Starting |
| Ex # | Name | Structure | [M + H]⁺ | Material |
| REF 11 | 4-[2-[[4- [[3-(3-fluoro-4- methoxy- phenyl) imidazo[1,2- a]pyrazin-8- yl]amino]-2- methyl-phenyl] methyl- methyl- amino]acetyl] piperazin-2- one | 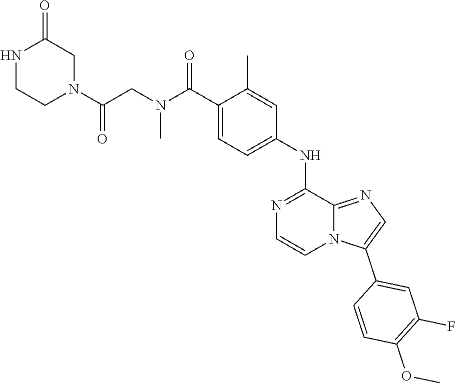 [SMILES: COc1ccc(-c2cnc3c(Nc4ccc(C(=O)N(C)CC(=O)N5CCNC(=O)C5)c(C)c4)nccn23)cc1F] | 546.1 | Intermediate 77 and piperazin- 2-one |
| REF 12 | N-[2- (dimethylamino)-2- oxo-ethyl]- 4-[[3-(3-fluoro- 4-methoxy- phenyl)imidazo[1,2- a]pyrazin-8- yl]amino]-N,2- dimethyl- benzamide | 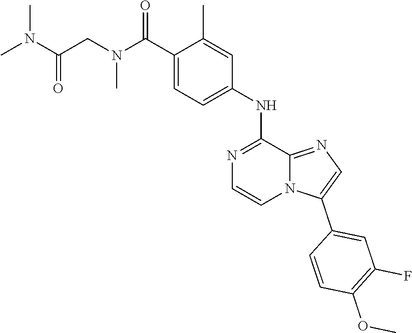 [SMILES: COc1ccc(-c2cnc3c(Nc4ccc(C(=O)N(C)CC(=O)N(C)C)c(C)c4)nccn23)cc1F] | 491.1 | Intermediate 77 and dimethylamine |
| REF 13 | 4-[[3-(3-fluoro- 4-methoxy- phenyl)imidazo[1,2- a]pyrazin-8- yl]amino]-N,2- dimethyl-N- (2-morpholino- 2-oxo-ethyl) benzamide | 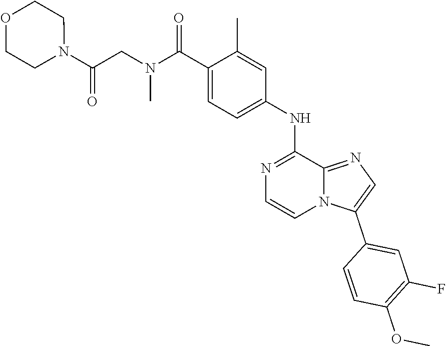 [SMILES: COc1ccc(-c2cnc3c(Nc4ccc(C(=O)N(C)CC(=O)N5CCOCC5)c(C)c4)nccn23)cc1F] | 533.1 | Intermediate 77 and morpholine |
| REF 14 | N-[2-[3- [(dimethylamino) methyl] pyrrolidin-1-yl]- 2-oxo-ethyl]-4- [[3-(3-fluoro- 4-methoxy- phenyl)imidazo[1,2- a]pyrazin-8-yl] amino]-N,2- dimethyl-benzamide | 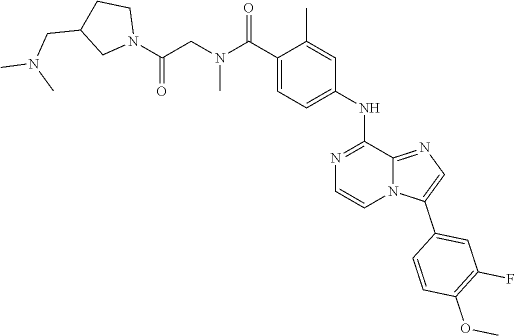 [SMILES: COc1ccc(-c2cnc3c(Nc4ccc(C(=O)N(C)CC(=O)N5CCC(CN(C)C)C5)c(C)c4)nccn23)cc1F] | 574.1 | Intermediate 77 and N,N- dimethyl-1- pyrro lidin-3- ylmethanamine dihydrochloride |

### Intermediate 78

### tert-Butyl N-[2-[2-[(4-amino-2-methyl-benzoyl)amino]ethoxy]ethyl]carbamate

### Step 1

### tert-Butyl N-[2-[2-[(2-methyl-4-nitro-benzoyl)amino]ethoxy]ethyl]carbamate

To a mixture of 2-methyl-4-nitro-benzoic acid (3.45 g, 19.04 mmol, 1 eq), N-Boc-2-(2-amino-ethoxy)-ethylamine (3.89 g, 19.04 mmol, 1 eq) and triethylamine (7.96 mL, 57.13 mmol, 3 eq) in THF (50 mL) was added 1-propanephosphonic anhydride in ethyl acetate (18.18 g, 28.57 mmol, 1.5 eq) at 25° C. The mixture was stirred at 25° C. for 16 h. The reaction was concentrated to dryness and the residue was taken up in ethyl acetate (50 mL) and washed with 2×50 mL water then 1×50 mL brine. The combined organic layers were then separated and dried (MgSO₄) before concentration to dryness to afford the crude product. The product was purified by silica gel column chromatography (30% ethyl acetate/PE) to afford the desired product (5.08 g) as a colorless oil.

### Step 2

### tert-Butyl N-[2-[2-[(4-amino-2-methyl-benzoyl)amino]ethoxy]ethyl]carbamate

A mixture of tert-butyl N-[2-[2-[(2-methyl-4-nitro-benzoyl)amino]ethoxy]ethyl]carbamate (2.0 g, 4.35 mmol, 1 eq) and palladium/C (1.5 mmol, 0.350 eq) in methanol (20 mL) was stirred under H₂ (775 mmHg) at 25° C. for 16 h. The mixture was filtered and purified by flash column chromatography to afford the title product (1.12 g) as a light yellow oil. MS (ESI, m/z): 238 [M+H-Boc]⁺.

### Intermediate 79

### tert-Butyl (2-(2-(4-((3-iodoimidazo[1,2-a]pyrazin-8-yl)amino)-2-methylbenzamido)ethoxy)ethyl)carbamate

The title compound was prepared in analogy to Reference Example 15 step 1 from Intermediate 1 and tert-butyl (2-(2-aminoethoxy)ethyl)carbamate. MS (ESI, m/z): 581.3[M+H]⁺

### Reference Example 16

### N-(2-(2-aminoethoxy)ethyl)-4-((3-(2,3-difluoro-4-methoxyphenyl)imidazo[1,2-a]pyrazin-8-yl)amino)-2-methylbenzamide Hydrochloride

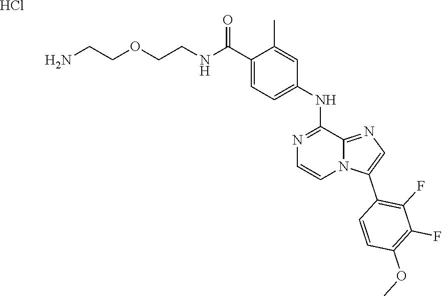 [SMILES: Cl ; COc1ccc(-c2cnc3c(Nc4ccc(C(=O)NCCOCCN)c(C)c4)nccn23)c(F)c1F]

### Step 1

### tert-Butyl (2-(2-(4-((3-iodoimidazo[1,2-a]pyrazin-8-yl)amino)-2-methylbenzamido)ethoxy)ethyl) Carbamate

tert-Butyl (2-(2-aminoethoxy)ethyl)carbamate (104 mg, 510 μmol), diisopropylethylamine (132 mg, 178 μl, 1.02 mmol) and HATU (259 mg, 680 μmol) were added to a solution of 4-((3-iodoimidazo[1,2-a]pyrazin-8-yl)amino)-2-methylbenzoic acid (intermediate 1, 134 mg, 340 μmol) in DMF (5 mL). The mixture was stirred overnight at room temperature. The reaction mixture was poured into 5 mL H₂O and extracted with acetonitrile. The organic layers were dried over sodium sulphate and concentrated in vacuo. The crude material was purified by flash chromatography (silica gel, 50% to 100% ethyl acetate in heptane) to give the title compound (112 mg) as a yellow solid. MS (ESI, m/z): 581.3 [M+H]⁺.

### Step 2

### tert-Butyl (2-(2-(4-((3-(2,3-difluoro-4-methoxyphenyl)imidazo[1,2-a]pyrazin-8-yl)amino)-2-methylbenzamido)ethoxy)ethyl)carbamate

tert-butyl (2-(2-(4-((3-iodoimidazo[1,2-a]pyrazin-8-yl)amino)-2-methylbenzamido)ethoxy)ethyl) carbamate (50 mg, 86.1 μmol), (2,3-difluoro-4-methoxyphenyl)boronic acid (24.3 mg, 129 μmol), Na₂CO₃ (18.3 mg, 172 μmol) and 1,1′-bis(diphenylphosphino)ferrocenedichloro palladium(II) dichloromethane complex (7.03 mg, 8.61 μmol) in dioxane (1000 μl) and water (100 μl) was heated in a microwave at 80° C. for 30 min. The crude reaction mixture was purified by prep. HPLC to give the title compound (28 mg) as a white solid. MS (ESI, m/z): 597.4 [M+H]⁺.

### Step 3

### N-(2-(2-aminoethoxy)ethyl)-4-((3-(2,3-difluoro-4-methoxyphenyl)imidazo[1,2-a]pyrazin-8-yl)amino)-2-methylbenzamide Hydrochloride

tert-Butyl (2-(2-(4-((3-(2,3-difluoro-4-methoxyphenyl)imidazo[1,2-a]pyrazin-8-yl)amino)-2-methylbenzamido)ethoxy)ethyl)carbamate (28 mg, 46.9 μmol) was combined with 3M HCl in MeOH (235 μl, 704 μmol) to give a light yellow solution. The reaction mixture was stirred at room temperature overnight. After removal of the volatiles, the solid obtained was dried in vacuo to give the title product (23.3 mg) as a light yellow solid. MS (ESI, m/z): 497.2 [M+H]⁺.

### Reference Example 15

### N-(2-(2-aminoethoxy)ethyl)-2-chloro-4-((3-(3-fluoro-4-methoxyphenyl)imidazo[1,2-a]pyrazin-8-yl)amino)benzamide Hydrochloride

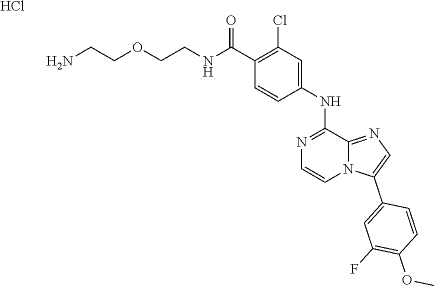 [SMILES: Cl ; COc1ccc(-c2cnc3c(Nc4ccc(C(=O)NCCOCCN)c(Cl)c4)nccn23)cc1F]

### Step 1

### tert-Butyl (2-(2-(2-chloro-4-((3-(3-fluoro-4-methoxyphenyl)imidazo[1,2-a]pyrazin-8-yl)amino)benzamido)ethoxy)ethyl) Carbamate

To 2-chloro-4-((3-(3-fluoro-4-methoxyphenyl)imidazo[1,2-a]pyrazin-8-yl)amino)benzoic acid (intermediate 2, 35 mg, 84.8 μmol) in DMF (1 mL) was added tert-butyl (2-(2-aminoethoxy)ethyl)carbamate (26 mg, 127 μmol), HATU (64.5 mg, 170 μmol) and diisopropylethylamine (32.9 mg, 44.4 μL, 254 μmol) followed by stirring at room temperature for 1 h. The crude reaction mixture was purified by prep. HPLC to give the title compound (31 mg) as an orange solid. MS (ESI, m/z): 599.4 [M+H]⁺.

### Step 2

### N-(2-(2-aminoethoxy)ethyl)-2-chloro-4-((3-(3-fluoro-4-methoxyphenyl)imidazo[1,2-a]pyrazin-8-yl)amino)benzamide Hydrochloride

The title compound was obtained as a white solid (31 mg) in analogy to Reference Example 16, step 3 from tert-butyl (2-(2-(2-chloro-4-((3-(3-fluoro-4-methoxyphenyl)imidazo[1,2-a]pyrazin-8-yl)amino)benzamido)ethoxy)ethyl) carbamate. MS (ESI, m/z): 500.3 [M+H]⁺.

The following examples were prepared in analogy to Reference Example 15, the deprotection step 2 was only applied for intermediates derived from Boc-protected amines.

| | | | MS | |
|---|---|---|---|---|
| | | | ESI | |
| | | | [M + | |
| Ex. | Name | Structure | H]⁺ | Starting Material |
| REF 17 | N-(6-aminohexyl)-4- ((3-(4-methoxy- phenyl)imida- zo[1,2-a]pyrazin-8- yl)amino)-2- methylbenzamide hydrochloride | 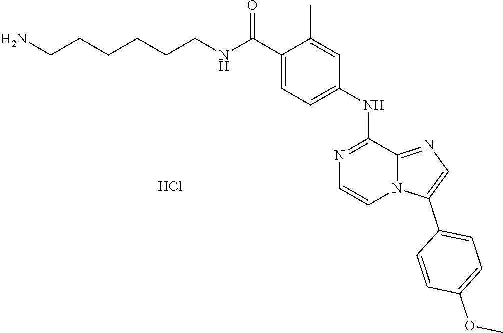 [SMILES: COc1ccc(-c2cnc3c(Nc4ccc(C(=O)NCCCCCCN)c(C)c4)nccn23)cc1 ; Cl] | 473.3 | Intermediate 4 and tert-butyl (6- aminohexyl)carbamate hydrochloride |
| REF 18 | (4-(2-Amino- ethyl)piperidin- 1-yl)(4-((3-(4-(di- fluoromethoxy)phe- nyl)imidazo[1,2- a]pyrazin-8- yl)amino)-2- methylphenyl)metha- none hydrochloride | 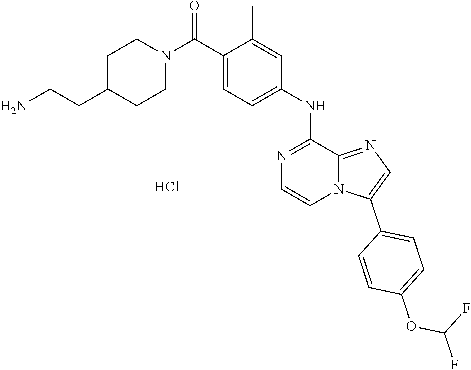 [SMILES: Cc1cc(Nc2nccn3c(-c4ccc(OC(F)F)cc4)cnc23)ccc1C(=O)N1CCC(CCN)CC1 ; Cl] | 521.1 | Intermediate 7 and tert-butyl (2- (piperidin-4- yl)ethyl)carbamate |
| REF 19 | 4-((3-(3-Fluoro-4- methoxy- phenyl)imida- zo[1,2-a]pyrazin-8- yl)amino)-N,2- dimethyl-N-(2- (piperazin-1- yl)ethyl)benzamide hydrochloride | 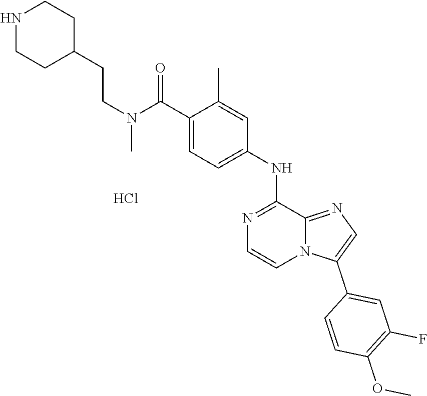 [SMILES: COc1ccc(-c2cnc3c(Nc4ccc(C(=O)N(C)CCC5CCNCC5)c(C)c4)nccn23)cc1F ; Cl] | 517.3 | Intermediate 6 and tert-butyl 4-(2- (methylamino)ethyl)- piperidine-1- carboxylate |
| 3 | (2-Chloro-4-((3-(3- fluoro-4-methoxy- phenyl)imida- zo[1,2-a]pyrazin-8- yl)amino)phenyl)(4- (2-(dimethyl- amino)ethyl) piperazin-1- yl)methanone | 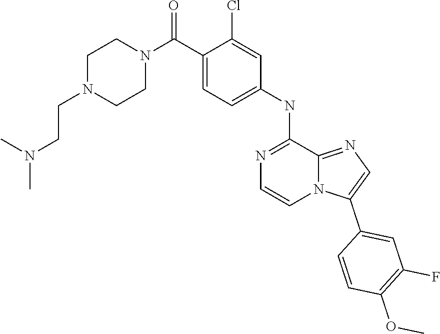 | 553.3 | Intermediate 2 and N,N-dimethyl-2- (piperazin-1- yl)ethanamine |
| REF 20 | 2-Chloro-4-((3-(3- fluoro-4-methoxy- phenyl)imida- zo[1,2-a]pyrazin-8- yl)amino)-N-methyl- N-(2-(piperazin-1- yl)ethyl)benzamide hydrochloride | 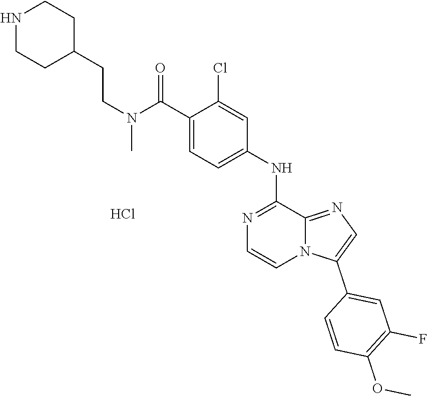 [SMILES: COc1ccc(-c2cnc3c(Nc4ccc(C(=O)N(C)CCC5CCNCC5)c(Cl)c4)nccn23)cc1F ; Cl] | 537.0 | Intermediate 2 and tert-butyl 4-(2- (methylamino)ethyl) piperazine-1- carboxylate |
| 4 | (4- (Aminomethyl)piper- idin-1-yl)(2-chloro- 4-((3-(3-fluoro-4- methoxyphenyl)imi- dazo[1,2-a]pyrazin- 8-yl)amino)phe- nyl)methanone hydrochloride | 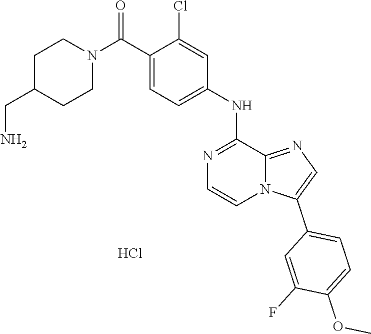 [SMILES: COc1ccc(-c2cnc3c(Nc4ccc(C(=O)N5CCC(CN)CC5)c(Cl)c4)nccn23)cc1F ; Cl] | 510.1 | Intermediate 2 and tert-butyl (piperidin- 4- methyl)carbamate |
| REF 21 | N-(2-(2- aminoethoxy)ethyl)- 2-chloro-4-((3-(4- methoxyphenyl)imi- dazo[1,2-a]pyra- zin-8- yl)amino)benzamide hydrochloride | 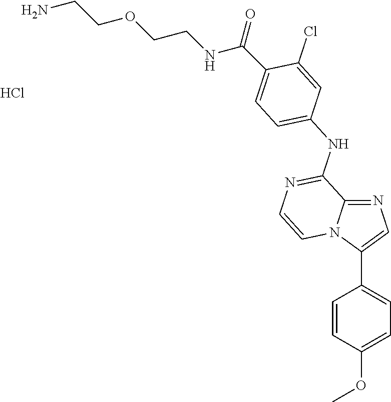 [SMILES: Cl ; COc1ccc(-c2cnc3c(Nc4ccc(C(=O)NCCOCCN)c(Cl)c4)nccn23)cc1] | 481.2 | Intermediate 9 and tert-butyl (2-(2- aminoethoxy)ethyl) carbamate |
| REF 22 | 1-(4-((3-(4-(di- fluoromethoxy)phe- nyl)imidazo[1,2- a]pyrazin-8- yl)amino)-2- methylbenzoyl)piper- idine-4-carboxamide | 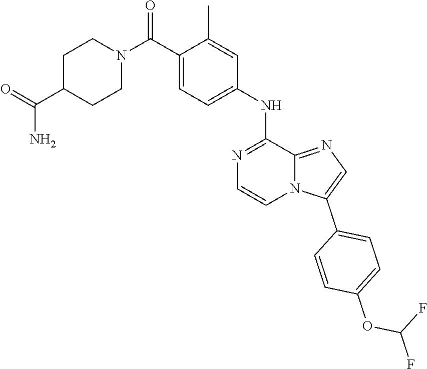 [SMILES: Cc1cc(Nc2nccn3c(-c4ccc(OC(F)F)cc4)cnc23)ccc1C(=O)N1CCC(C(N)=O)CC1] | 521.7 | Intermediate 7 and piperidine-4- carboxamide |
| REF 23 | (4-((3-(4-methoxy- phenyl)imida- zo[1,2-a]pyrazin-8- yl)amino)-2- methylphenyl)(4- methylpiperazin-1- yl)methanone | 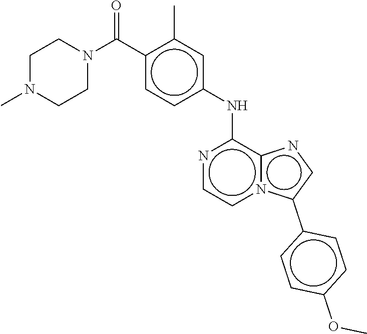 | 456 | Intermediate 4 and 1- methylpiperazine |
| REF 24 | N-(2-((2- hydroxyethyl)amino) ethyl)-4-((3-(4- methoxy- phenyl)imida- zo[1,2-a]pyrazin-8- yl)amino)-2- methylbenzamide | 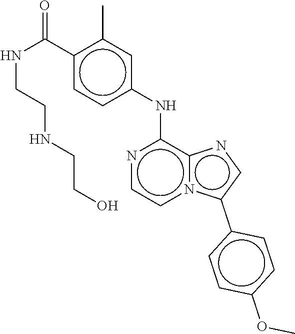 | 460 | Intermediate 4 and 2- ((2- aminoethyl)amino)eth- anol |
| REF 25 | 4-((3-(4-methoxy- phenyl)imida- zo[1,2-a]pyrazin-8- yl)amino)-N,N,2- trimethylbenzamide | 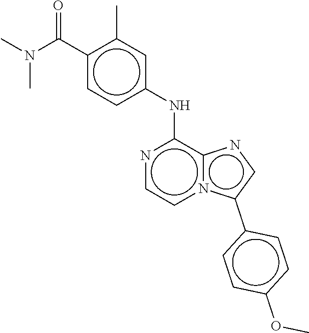 | 401 | Intermediate 4 and dimethylamine hydrochloride |
| REF 26 | (4-((3-(4-methoxy- phenyl)imida- zo[1,2-a]pyrazin-8- yl)amino)-2- methylphenyl)(mor- pholino)methanone | 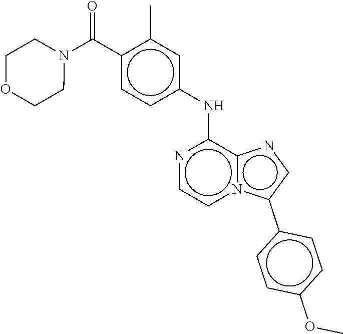 | 443 | Intermediate 4 and morpholine |
| REF 27 | (4-hydroxypiperidin- 1-yl)(4-((3-(4-meth- oxyphenyl)imida- zo[1,2-a]pyrazin-8- yl)amino)-2- methylphenyl)metha- none | 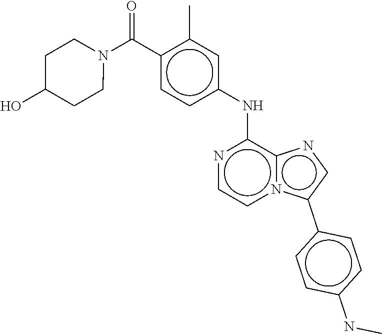 | 457 | Intermediate 4 and piperidin-4-ol hydrochloride |
| REF 28 | 4-((3-(4-methoxy- phenyl)imida- zo[1,2-a]pyrazin-8- yl)amino)-N,2- dimethylbenzamide | 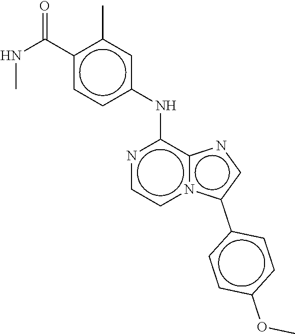 | 387 | Intermediate 4 and methanamine hydrochloride |
| REF 29 | 4-((3-(4-meth- oxyphenyl)imida- zo[1,2-a]pyrazin-8- yl)amino)-2-methyl- N-(1- methylpiperidin-4- yl)benzamide | 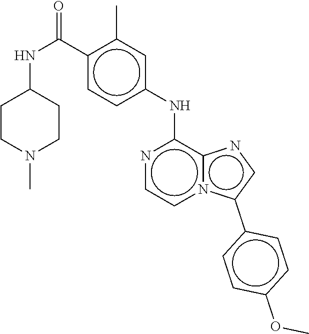 | 470 | Intermediate 4 and 1- methylpiperidin-4- amine hydrochloride |
| REF 30 | 4-((3-(4-methoxy- phenyl)imida- zo[1,2-a]pyrazin-8- yl)amino)-2-methyl- N-(2-(methylamino)- 2- oxoethyl)benzamide | 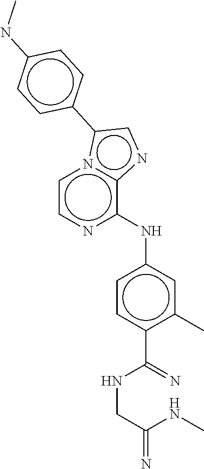 | 444 | Intermediate 4 and 2- amino-N- methylacetamide hydrochloride |
| REF 31 | N-(2- (dimethylamino)-2- oxoethyl)-4-((3-(4- methoxy- phenyl)imida- zo[1,2-a]pyrazin-8- yl)amino)-2- methylbenzamide | 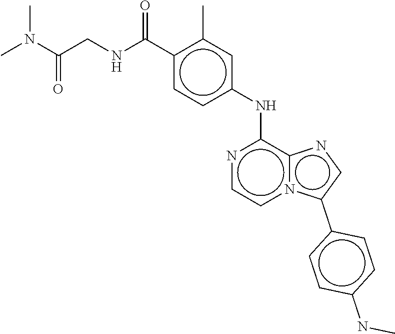 | 458 | Intermediate 4 and 1,1-dimethylurea hydrochloride |
| REF 32 | N-(2-amino-2- oxoethyl)-4-((3-(4- methoxy- phenyl)imida- zo[1,2-a]pyrazin-8- yl)amino)-2- methylbenzamide | 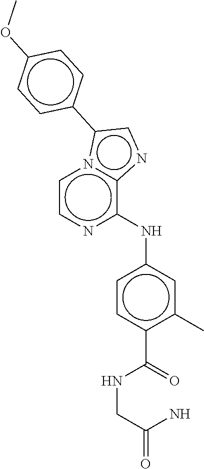 | 430 | Intermediate 4 and 2- aminoacetamide hydrochloride |
| REF 33 | N-(2- (dimethylamino)eth- yl)-4-((3-(4- methoxy- phenyl)imida- zo[1,2-a]pyrazin-8- yl)amino)-N,2- dimethylbenzamide | 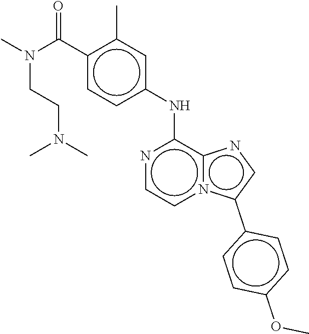 | 458 | Intermediate 4 and N1,N1,N2- trimethylethane-1,2- diamine |
| REF 34 | N-(2-hydroxyethyl)- 4-((3-(4-methoxy- phenyl)imida- zo[1,2-a]pyrazin-8- yl)amino)-N,2- dimethylbenzamide | 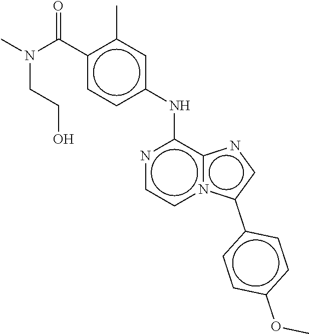 | 431 | Intermediate 4 and 2- (methylamino)ethanol |
| REF 35 | N-(2-(4- hydroxypiperidin-1- yl)-2-oxoethyl)-4- ((3-(4-methoxy- phenyl)imida- zo[1,2-a]pyrazin-8- yl)amino)-2- methylbenzamide | 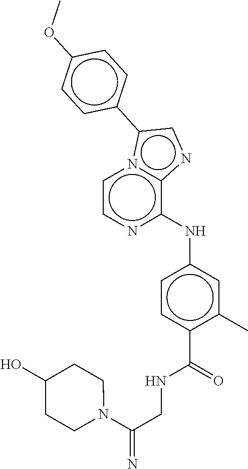 | 514 | Intermediate 4 and 2- amino-1-(4- hydroxypiperidin-1- yl)ethanone |
| REF 36 | N-(6-aminohexyl)-4- ((3-(4-(di- fluoromethoxy)phe- nyl)imidazo[1,2- a]pyrazin-8- yl)amino)-2- methylbenzamide hydrochloride | 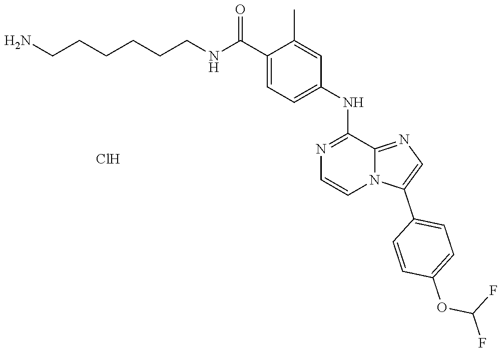 [SMILES: Cc1cc(Nc2nccn3c(-c4ccc(OC(F)F)cc4)cnc23)ccc1C(=O)NCCCCCCN ; Cl] | 509.2 | Intermediate 7 and tert-butyl (6- aminohexyl)carbamate |
| REF 37 | (4-(1H-1,2,4-triazol- 1-yl)piperidin-1- yl)(4-((3-(4-(di- fluoromethoxy)phe- nyl)imidazo[1,2- a]pyrazin-8- yl)amino)-2- methylphenyl)metha- none | 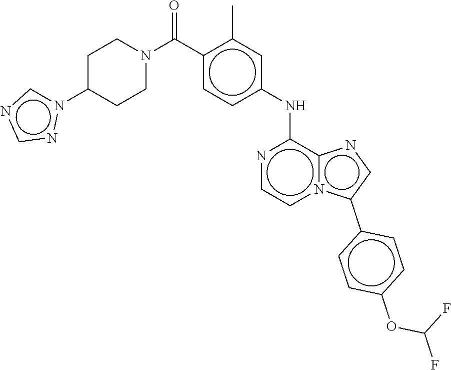 | 545.6 | Intermediate 7 and 4-(1H-1,2,4-triazol-1- yl)piperidine |
| REF 38 | 1-(1-(4-((3-(4- (difluoromethoxy) phenyl)imidazo[1,2- a]pyrazin-8- yl)amino)-2- methylbenzoyl)pipe- ridin-4- yl)imidazolidin-2- one | 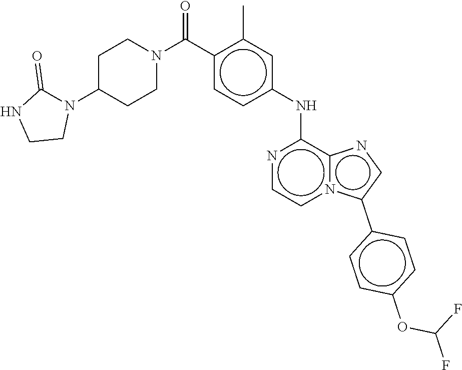 | 562.7 | Intermediate 7 and 1-(piperidin-4- yl)imidazolidin-2-one |
| REF 22 | 1-(4-((3-(4-(di- fluoromethoxy)phe- nyl)imidazo[1,2- a]pyrazin-8- yl)amino)-2- methylbenzoyl)piper- idine-4- carboxamide | 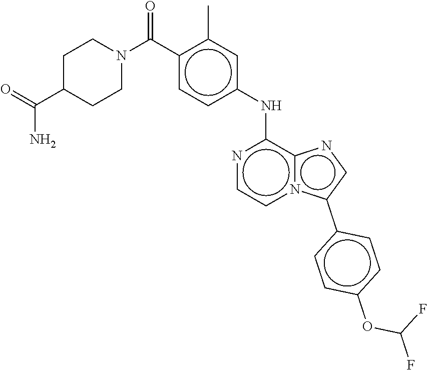 | 521.7 | Intermediate 7 and piperidine-4- carboxamide |
| REF 39 | 1-(4-((3-(4- (difluoromethoxy) phenyl)imidazo[1,2- a]pyrazin-8- yl)amino)-2-methyl- benzoyl)piperi- dine-3-carboxamide | 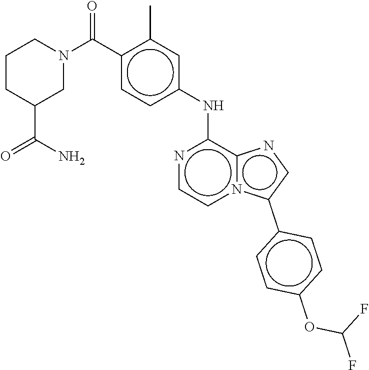 | 521.2 | Intermediate 7 and piperidine-3- carboxamide |
| REF 40 | ethyl (1-(4-((3-(4- (difluoromethoxy) phenyl)imidazo[1,2- a]pyrazin-8- yl)amino)-2-methyl- benzoyl)piperi- din-4-yl)carbamate | 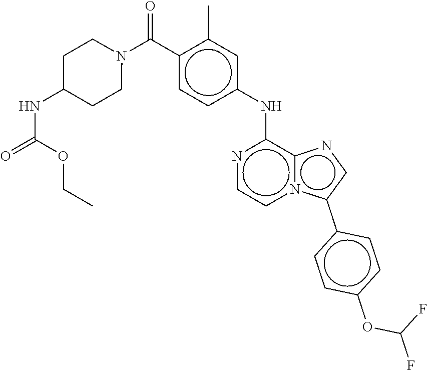 | 565.4 | Intermediate 7 and ethyl piperidin-4- ylcarbamate |
| REF 41 | (4-((3-(4- (difluoromethoxy) phenyl)imidazo[1,2- a]pyrazin-8- yl)amino)-2- methylphenyl)(4- (pyrimidin-2- yloxy)piperidin-1- yl)methanone | 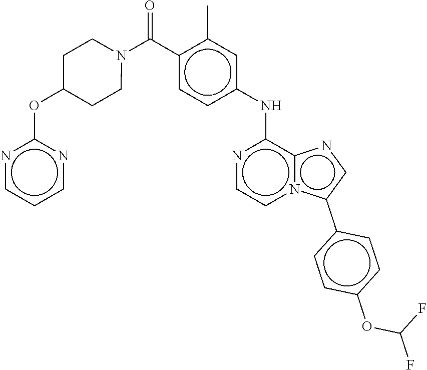 | 572.5 | Intermediate 7 and 2-(piperidin-4- yloxy)pyrimidine dihydrochloride |
| REF 42 | (4-((3-(4- (difluoromethoxy) phenyl)imidazo[1,2- a]pyrazin-8- yl)amino)-2- methylphenyl)(4-(4- methyl-4H-1,2,4- triazol-3- yl)piperidin-1- yl)methanone | 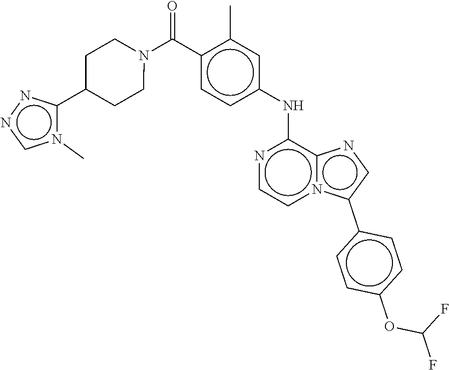 | 559.5 | Intermediate 7 and 4-(4-methyl-4H- 1,2,4-triazol-3- yl)piperidine |
| REF 43 | 2-chloro-4-((3-(3- fluoro-4-methoxy- phenyl)imida- zo[1,2-a]pyrazin-8- yl)amino)-N- methylbenzamide | 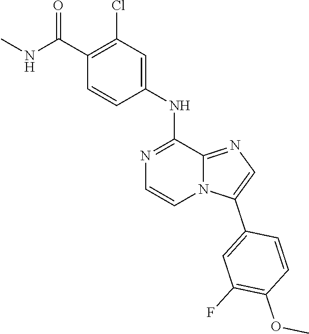 | 426.3 | Intermediate 2 and methylamine hydrochloride |
| REF 44 | (1-(4-((3-(3-fluoro- 4-methoxy- phenyl)imida- zo[1,2-a]pyrazin-8- yl)amino)-2-methyl- benzoyl)piperi- din-4-yl)(piperazin- 1-yl)methanone hydrochloride | 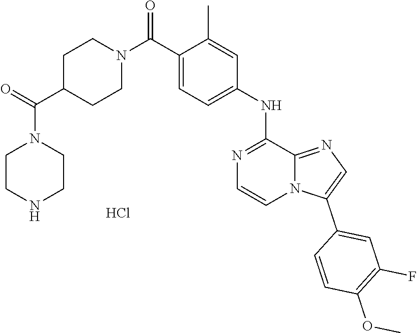 [SMILES: COc1ccc(-c2cnc3c(Nc4ccc(C(=O)N5CCC(C(=O)N6CCNCC6)CC5)c(C)c4)nccn23)cc1F ; Cl] | 572.3 | Intermediate 6 and tert-butyl piperazine- 1-carboxylate |
| 5 | (4-(amino- methyl)piperi- din-1-yl)(2-bromo- 4-((3-(4-(di- fluoromethoxy)phe- nyl)imidazo[1,2- a]pyrazin-8- yl)amino)phenyl) methanone hydrochloride | 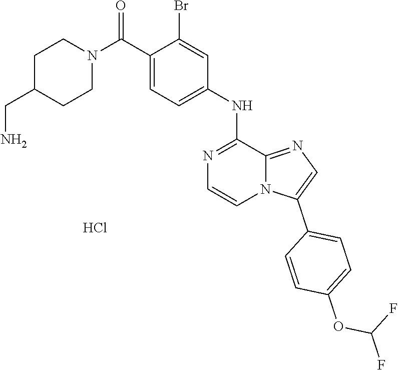 [SMILES: NCC1CCN(C(=O)c2ccc(Nc3nccn4c(-c5ccc(OC(F)F)cc5)cnc34)cc2Br)CC1 ; Cl] | 573.3 | Intermediate 10 and tert-butyl (piperidin- 4- ylmethyl)carbamate |
| REF 45 | 2-chloro-4-((3-(4- (difluorometh- oxy)phenyl)imida- zo[1,2-a]pyrazin-8- yl)amino)-N- methyl- N-(2-(piperidin-4- yl)ethyl)benzamide hydrochlroide | 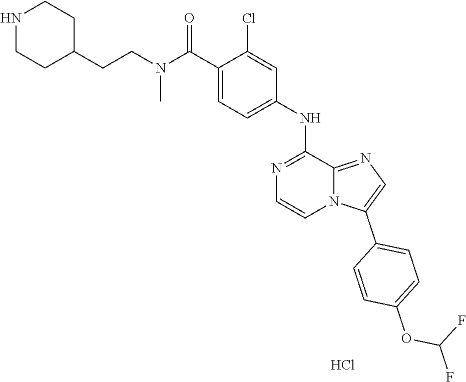 [SMILES: CN(CCC1CCNCC1)C(=O)c1ccc(Nc2nccn3c(-c4ccc(OC(F)F)cc4)cnc23)cc1Cl ; Cl] | 555.2 | Intermediate 11 and tert-butyl 4-(2- (methylamino)ethyl) piperidine-1- carboxylate |
| REF 46 | 1-(4-((3-(4-(di- fluoromethxoy)phe- nyl)imidazo[1,2- a]pyrazin-8- yl)amino)-2- methylbenzoyl)-N- methylpiperidine-4- carboxamide | 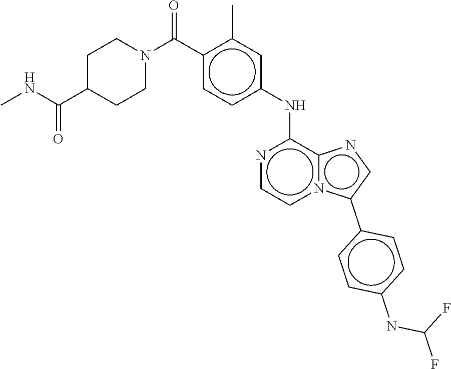 | 535.3 | Intermediate 7 and N- methylpiperidine-4- carboxamide |
| REF 47 | N-((1-carba- mimidoylpiperi- din-4-yl)methyl)-4- ((3-(4-(di- fluoromethoxy)phe- nyl)imidazo[1,2- a]pyrazin-8- yl)amino)-2- methylbenzamide | 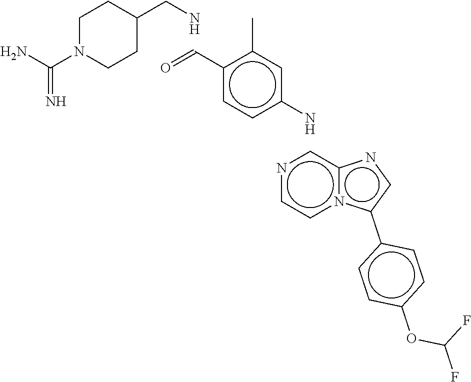 | 549.3 | Intermediate 7 and 4- (aminomethyl)piperi- dine-1- carboximidazmide hydrochloride |
| REF 48 | (4-((1H-pyrazol-1- yl)methyl)piperidin- 1-yl)(4-((3-(4-(di- fluoromethoxy)phe- nyl)imidazo[1,2- a]pyrazin-8- yl)amino)-2- methylphenyl)meth- anone | 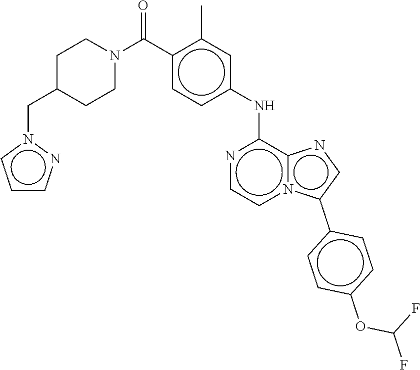 | 558.2 | Intermediate 7 and 4- ((1H-pyrazol-1- yl)methyl)piperidine |
| REF 49 | (4-((3-(4-(di- fluoromethoxy)phe- nyl)imidazo[1,2- a]pyrazin-8- yl)amino)-2- methylphenyl)(1- oxa-4,9- diazaspiro[5.5]unde- can-9-yl)methanone | 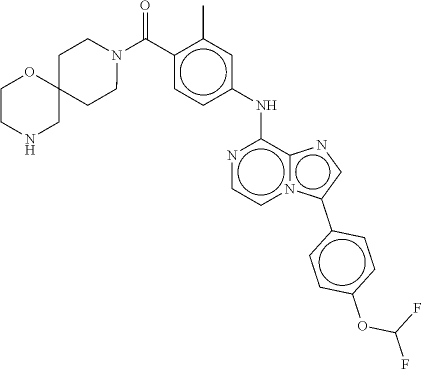 | 549.2 | Intermediate 7 and 1- oxa-4,9- diazaspiro[5.5]unde- cane |
| REF 50 | 4-((3-(4-(di- fluoromethoxy)phe- nyl)imidazo[1,2- a]pyrazin-8- yl)amino)-2-methyl- N-(quinuclidin-3- yl)benzamide | 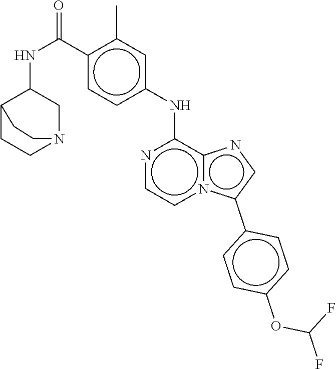 | 519.3 | Intermediate 7 and quinuclidin-3-amine dihydrochloride |
| REF 51 | N-(2-(2- chloroethoxy)ethyl)- 4-((3-(4-methoxy- phenyl)imida- zo[1,2-a]pyrazin-8- yl)amino)-2- methylbenzamide | 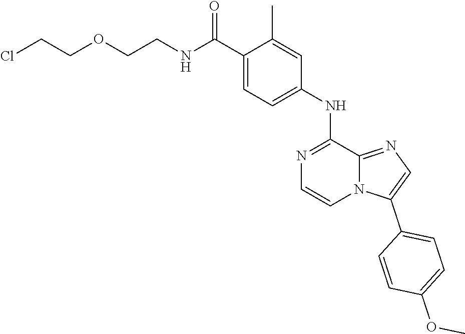 [SMILES: COc1ccc(-c2cnc3c(Nc4ccc(C(=O)NCCOCCCl)c(C)c4)nccn23)cc1] | 480.3 | Intermediate 4 and 2- (2- chloroethoxy)ethana- mine hydrochloride |
| REF 52 | (4-(amino- methyl)piperi- din-1-yl)(4-((3-(4- (difluoro- methoxy)phe- nyl)imidazo[1,2- a]pryazin-8- yl)amino)-2-meth- ylphenyl)metha- none hydrochloride | 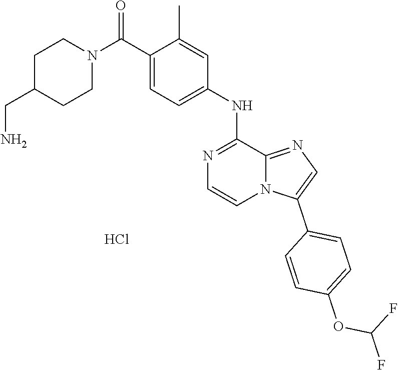 [SMILES: Cc1cc(Nc2nccn3c(-c4ccc(OC(F)F)cc4)cnc23)ccc1C(=O)N1CCC(CN)CC1 ; Cl] | 505.4 [M − H]⁺ | Intermediate 7 and tert-butyl(piperidin- 4- ylmethyl)carbamate |
| REF 53 | (4-(amino- methyl)piperi- din-1-yl)(4-((3-(4- methoxyphenyl)imi- dazo[1,2-a]pyrazin- 8-yl)amino)-2-meth- ylphenyl)metha- none hydrochloride | 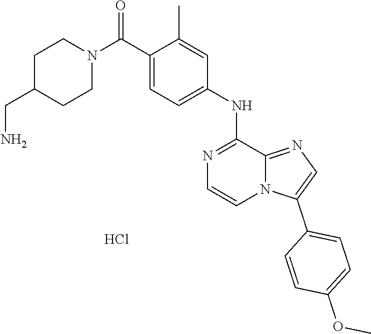 [SMILES: COc1ccc(-c2cnc3c(Nc4ccc(C(=O)N5CCC(CN)CC5)c(C)c4)nccn23)cc1 ; Cl] | 508.0 | Intermediate 4 and tert-butyl (piperidin- 4- ylmethyl)carbamate |
| REF 54 | (4-(azetidin-3- yl)piperidin-1-yl)(4- ((3-(4-(di- fluoromethoxy)phe- nyl)imidazo[1,2- a]pyrazin-8- yl)amino)-2-meth- ylphenyl)metha- none hydrochloride | 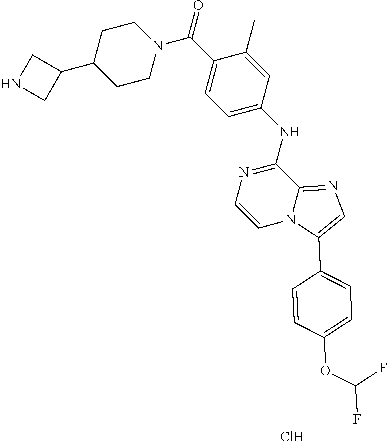 [SMILES: Cc1cc(Nc2nccn3c(-c4ccc(OC(F)F)cc4)cnc23)ccc1C(=O)N1CCC(C2CNC2)CC1 ; Cl] | 533.2 | Intermediate 7 and tert-butyl 3- (piperidin-4- yl)azetidine-1- carboxylate |
| REF 55 | N-(2-(2- aminoethoxy)ethyl)- 4-((3-(4-chloro-2,3- difluorophenyl)imi- dazo[1,2-a]pyrazin- 8-yl)amino)-2- ethylbenzamide hydrochloride | 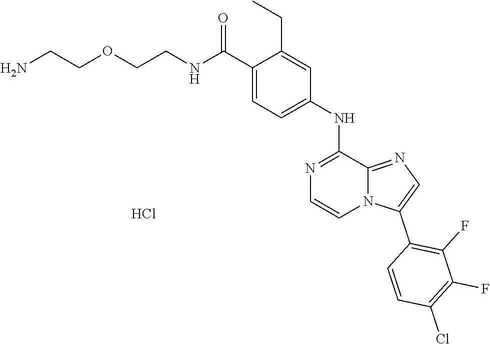 [SMILES: CCc1cc(Nc2nccn3c(-c4ccc(Cl)c(F)c4F)cnc23)ccc1C(=O)NCCOCCN ; Cl] | 515.1 | Intermediate 12 and tert-butyl (2- (2- aminoethoxy)ethyl) carbamate |
| REF 56 | 4-((3-(4-(di- fluoromethoxy)phe- nyl)imidazo[1,2- a]pyrazin-8- yl)amino)-2-methyl- N-(5-morpholino- pentyl)benzamide | 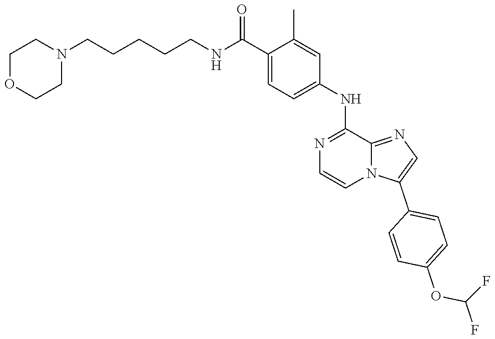 [SMILES: Cc1cc(Nc2nccn3c(-c4ccc(OC(F)F)cc4)cnc23)ccc1C(=O)NCCCCCN1CCOCC1] | 565.2 | Intermediate 7 and 5- morpholinopentan-1- amine |
| REF 57 | N-(2-(2-amino-2- oxoethoxy)ethyl)-4- ((3-(4-(di- fluoromethoxy)phe- nyl)imidazo[1,2- a]pryazin-8- yl)amino)-2- methylbenzamide | 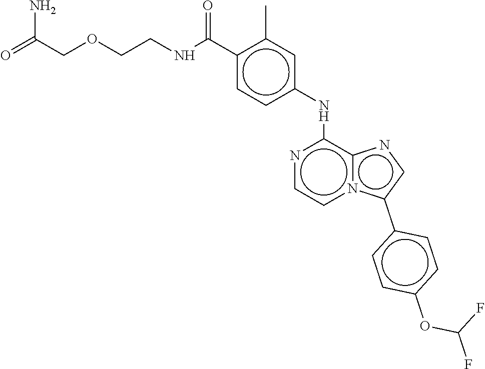 | 511.4 | Intermediate 7 and 2- (2-amino- ethoxy)acetamide |
| REF 58 | 4-((3-(4-(di- fluoromethoxy)phe- nyl)imidazo[1,2- a]pyrazin-8- yl)amino)-2-methyl- N-(2-(2- (methylamino)-2- oxoethoxy)eth- yl)benzamide | 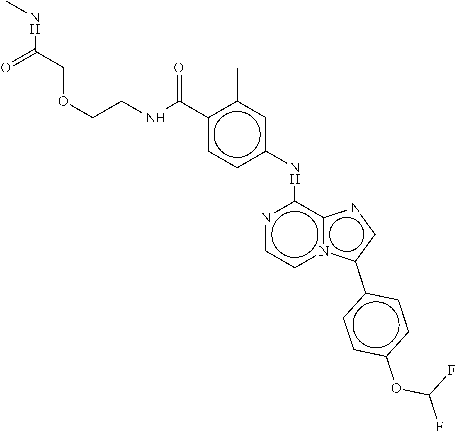 | 525.4 | Intermediate 7 and 2- (2-aminoethoxy)-N- methylacetamide |
| REF 59 | 4-((3-(4-(di- fluoromethoxy)phe- nyl)imidazo[1,2- a]pyrazin-8- yl)amino)-N-(2-(2- (2,4-dioxo- oxazolidin-3- yl)ethoxy)ethyl)-2- methylbenzamide | 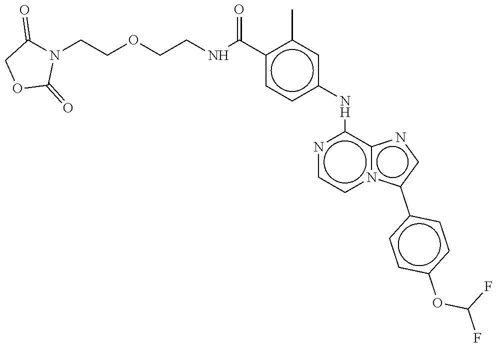 | 581.4 | Intermediate 7 and 3- (2-(2-amino- ethoxy)ethyl)oxa- zolidine-2,4-dione |
| REF 60 | 4-((3-(4-(di- fluoromethoxy)phe- nyl)imidazo[1,2- a]pyrazin-8- yl)methyl)-N,2- dimethylbenzamide | 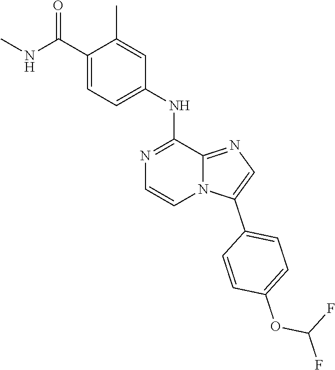 [SMILES: CNC(=O)c1ccc(Nc2nccn3c(-c4ccc(OC(F)F)cc4)cnc23)cc1C] | 422 [M − H]⁻ | Intermediate 7 and methylamine hydrochloride |
| REF 61 | 4-((3-(3-chloro-4- methoxy- phenyl)imida- zo[1,2-a]pyrazin-8- yl)amino)-N,2- dimethylbenzamide | 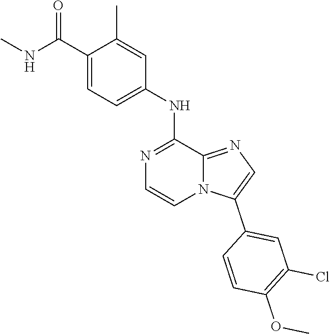 [SMILES: CNC(=O)c1ccc(Nc2nccn3c(-c4ccc(OC)c(Cl)c4)cnc23)cc1C] | 422.3 | Intermediate 14 and methylamine (2M in THF) |
| REF 62 | (4-((3-(3-chloro-4- methoxy- phenyl)imida- zo[1,2-a]pyrazin-8- yl)amino)-2-methyl- phenyl)(morpho- lino)methanone | 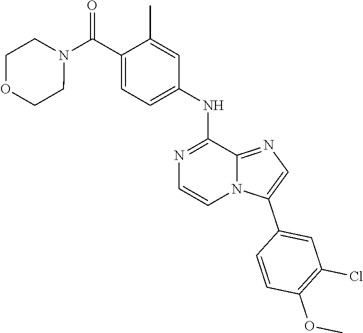 [SMILES: COc1ccc(-c2cnc3c(Nc4ccc(C(=O)N5CCOCC5)c(C)c4)nccn23)cc1Cl] | 478.2 | Intermediate 14 and morpholine |
| REF 63 | 4-((3-(3-chloro-4- methoxy- phenyl)imida- zo[1,2-a]pyrazin-8- yl)amino)-N-(2- (dimethyl- amino)ethyl)-N- methylbenzamide | 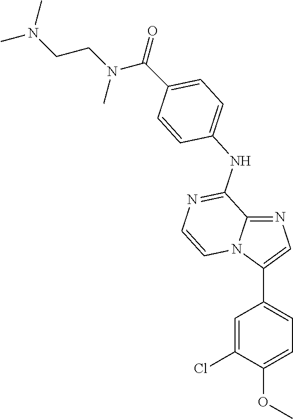 | 479.7 | Intermediate 16 and N1,N1,N2- trimethylethane-1,2- diamine |
| REF 64 | N-(2-aminoethyl)-4- [[3-(3-chloro-4- methoxyphenyl)imi- dazo[1,2-a]pyrazin- 8-yl]amino]benz- amide; hydrochloride | 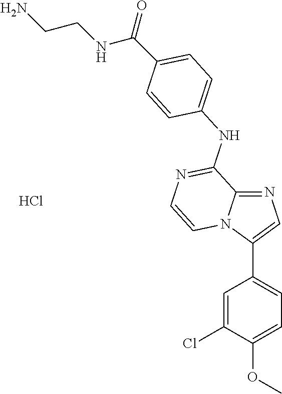 [SMILES: COc1ccc(-c2cnc3c(Nc4ccc(C(=O)NCCN)cc4)nccn23)cc1Cl ; Cl] | 435.3 | Interemdiate 16 and tert-butyl (2- aminoethyl)carbamate |
| REF 65 | 4-[[3-(3-chloro-4- methoxyphenyl)imi- dazo[1,2-a]pyrazin- 8-yl]amino]-N-[2- (methylamino)ethyl] benzamide; hydrochloride | 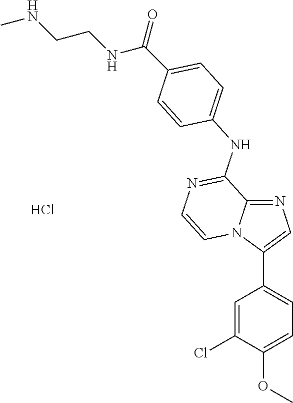 | 449.3 | Intermediate 16 and tert-butyl (2- aminoethyl)(methyl) carbamate |
| REF 66 | 4-((3-(3-chloro-4- methoxyphenyl)imi- dazo[1,2-a]pyrazin- 8-yl)amino)-N-(2- (piperazin-1- yl)ethyl)benzamide hydrochloride | 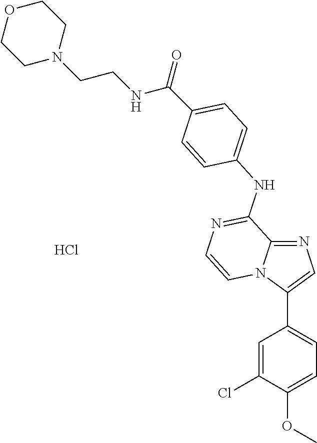 [SMILES: COc1ccc(-c2cnc3c(Nc4ccc(C(=O)NCCN5CCOCC5)cc4)nccn23)cc1Cl ; Cl] | 506.2 | Intermediate 16 and tert-butyl 4-(2-amino- ethyl)piperazine- 1-carboxylate |
| REF 67 | (4-((3-(3-chloro-4- methoxyphenyl)imi- dazo[1,2-a]pyrazin- 8-yl)amino)-2- methylphenyl)(4- methylpiperazin-1- yl)methanone | 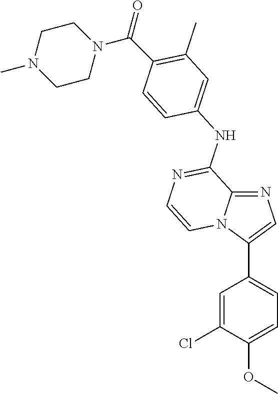 | 389.4 [M − H]⁻ | Intermediate 14 and 1- methylpiperazine |
| REF 68 | N-(2-aminoethyl)-4- ((3-(4-methoxy- phenyl)imida- zo[1,2-a]pyrazin-8- yl)amino)-2- methylbenzamide | 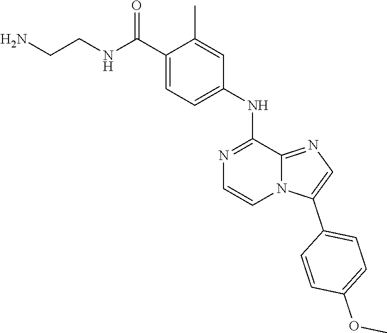 [SMILES: COc1ccc(-c2cnc3c(Nc4ccc(C(=O)NCCN)c(C)c4)nccn23)cc1] | 417.2 | Intermediate 4 and tert-butyl (2- aminoethyl)carbamate |
| REF 69 | N-(2-aminoethyl)-4- ((3-(4-methoxy- phenyl)imida- zo[1,2-a]pyrazin-8- yl)amino)-N,2- dimethylbenzamide | 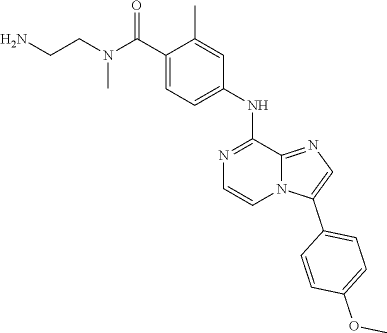 [SMILES: COc1ccc(-c2cnc3c(Nc4ccc(C(=O)N(C)CCN)c(C)c4)nccn23)cc1] | 431.2 | Intermediate 4 and tert-butyl (2- (methylamino)ethyl) carbamate |
| REF 70 | N-(3-aminopropyl)- 4-((3-(4-methoxy- phenyl)imida- zo[1,2-a]pyrazin-8- yl)amino)-N- methylbenzamide hydrochloride | 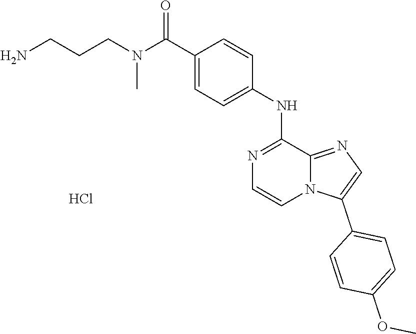 [SMILES: COc1ccc(-c2cnc3c(Nc4ccc(C(=O)N(C)CCCN)cc4)nccn23)cc1 ; Cl] | 431.2 | Intermediate 17 and tert-butyl (3- (methylamino)propyl) carbamate hydrochloride |
| REF 71 | N-(3-aminopropyl)- 4-((3-(4-methoxy- phenyl)imida- zo[1,2-a]pyrazin-8- yl)amino)-N,2- dimethylbenzamide hydrochloride | 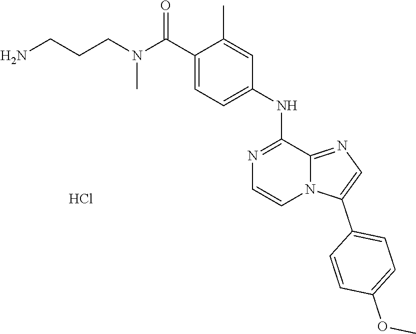 [SMILES: COc1ccc(-c2cnc3c(Nc4ccc(C(=O)N(C)CCCN)c(C)c4)nccn23)cc1 ; Cl] | 445.2 | Intermediate 4 and tert-butyl (3- (methylamino)propyl) carbamate hydrochloride |
| REF 72 | 4-[[3-(3-fluoro-4- methoxy- phenyl)imidazo[1,2- a]pyrazin-8- yl]amino]-N,2- dimethyl-N- (tetrahydropyran-4- ylmethyl)benzamide; 2,2,2-trifluoroacetic acid | 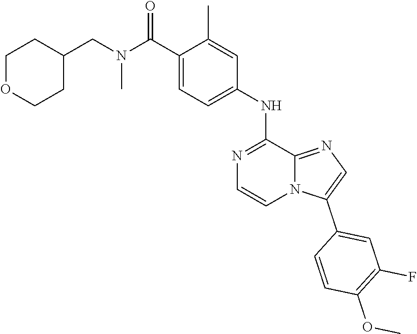 [SMILES: COc1ccc(-c2cnc3c(Nc4ccc(C(=O)N(C)CC5CCOCC5)c(C)c4)nccn23)cc1F] | 504.1 | Intermediate 6 and N- methyl-1-(tetrahydro- 2H-pyran-4- yl)methanamine |
| REF 73 | [2-[(di- methylamino)meth- yl]morpholin-4- yl]-[4-[[3-(3-fluoro- 4-methoxy- phenyl)imidazo[1,2- a]pyrazin-8- yl]amino]-2-methyl- phenyl]methanone; 2,2,2-trifluoroacetic acid | 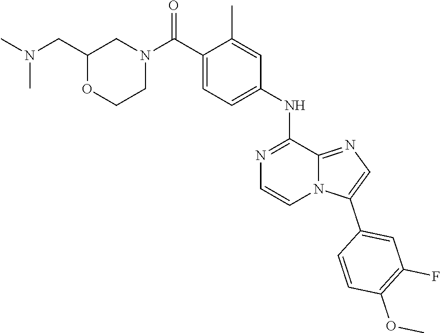 [SMILES: COc1ccc(-c2cnc3c(Nc4ccc(C(=O)N5CCOC(CN(C)C)C5)c(C)c4)nccn23)cc1F] | 519.1 | Intermediate 6 and N,N-dimethyl-1- (morpholin-2- yl)methanamine |
| REF 74 | N-[3-(1,1-dioxo-1,4- thiazinan-4- yl)propyl]-4-[[3-(3- fluoro-4-methoxy- phenyl)imidazo[1,2- a]pyrazin-8- yl]amino]-2-methyl- benzamide; 2,2,2- trifluoroacetic acid | 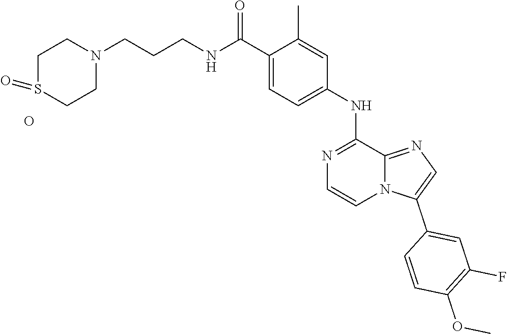 | 567.2 | Intermediate 6 and 4- (3- aminopropyl)thiomor- pholine 1,1-dioxide |
| REF 75 | 4-[[3-(3-fluoro-4- methoxy- phenyl)imidazo[1,2- a]pyrazin-8- yl]amino]-2-methyl- N-(2- tetrahydropyran-4- ylethyl)benzamide; 2,2,2-trifluoroacetic acid | 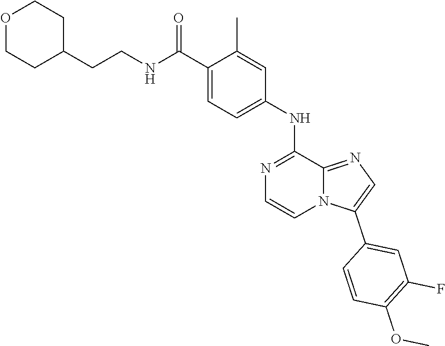 [SMILES: COc1ccc(-c2cnc3c(Nc4ccc(C(=O)NCCC5CCOCC5)c(C)c4)nccn23)cc1F] | 504.2 | Intermediate 6 and 2- (tetrahydro-2H- pyran-4- yl)ethanamine |
| REF 76 | 4-[[3-(3-fluoro-4- methoxy- phenyl)imidazo[1,2- a]pyrazin-8- yl]amino]-N,2- dimethyl-N-(3- pyridylmethyl)ben- zamide | 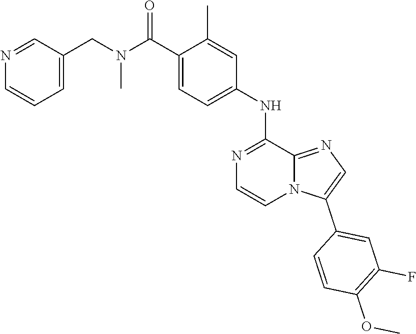 [SMILES: COc1ccc(-c2cnc3c(Nc4ccc(C(=O)N(C)Cc5cccnc5)c(C)c4)nccn23)cc1F] | 497.3 | Intermediate 6 and N- methyl-N-(3- pyridylmethyl)amine |
| REF 77 | 4-[[3-(3-fluoro-4- methoxy- phenyl)imidazo[1,2- a]pyrazin-8- yl]amino]-N,2- dimethyl-N- (pyrimidin-4- ylmethyl)benzamide | 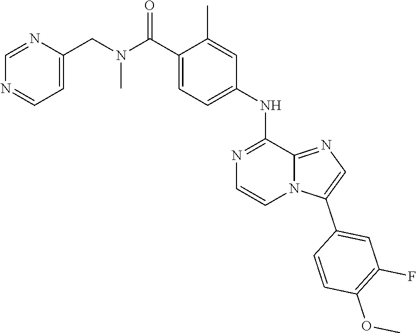 [SMILES: COc1ccc(-c2cnc3c(Nc4ccc(C(=O)N(C)Cc5ccncn5)c(C)c4)nccn23)cc1F] | 498.0 | Intermediate 6 and N- methyl-1-pyrimidin- 4-yl-methanamine |
| REF 78 | 2-chloro-4-[[3-(2,3- difluoro-4-methoxy- phenyl)imidazo[1,2- a]pyrazin-8- yl]amino]-N- (tetrahydropyran-4- ylmethyl)benzamide; 2,2,2-trifluoroacetic acid | 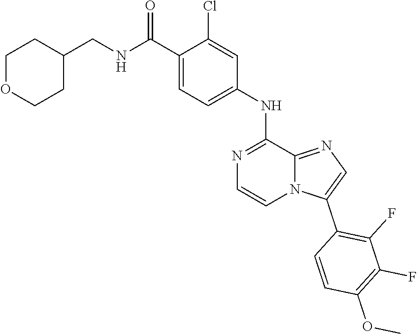 [SMILES: COc1ccc(-c2cnc3c(Nc4ccc(C(=O)NCC5CCOCC5)c(Cl)c4)nccn23)c(F)c1F] | 528.1 | Intermediate 20 and (tetrahydro-2H- pyran-4- yl)methanamine |
| REF 79 | 2-chloro-4-[[3-(2,3- difluoro-4-methoxy- phenyl)imidazo[1,2- a]pyrazin-8- yl]amino]-N-methyl- N-(tetrahydropyran- 4- ylmethyl)benzamide; 2,2,2-trifluoroacetic acid | 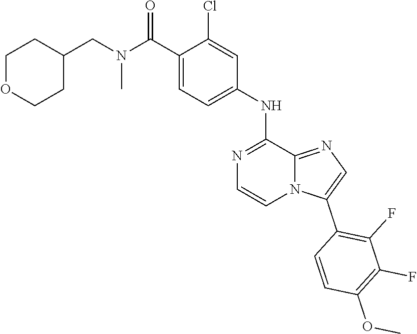 [SMILES: COc1ccc(-c2cnc3c(Nc4ccc(C(=O)N(C)CC5CCOCC5)c(Cl)c4)nccn23)c(F)c1F] | 542.1 | Intermediate 20 and N-methyl-1- (tetrahydro-2H- pyran-4- yl)methanamine |
| REF 80 | 2-chloro-4-[[3-(2,3- difluoro-4-methoxy- phenyl)imidazo[1,2- a]pyrazin-8- yl]amino]-N- (tetrahydrothiopyran- 4- ylmethyl)benzamide | 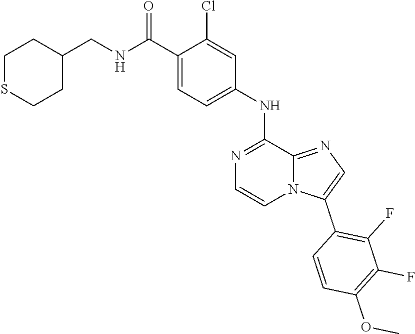 [SMILES: COc1ccc(-c2cnc3c(Nc4ccc(C(=O)NCC5CCSCC5)c(Cl)c4)nccn23)c(F)c1F] | 544.2 | Intermediate 20 and (tetrahydro-2H- thiopyran-4- yl)methanamine |
| REF 81 | [4-(2-amino- ethyl)piperazin- 1-yl]-[4-[[3-[4-(di- fluoromethoxy)phe- nyl]imidazo[1,2- a]pyrazin-8- yl]amino]-2-methyl- phenyl]methanone | 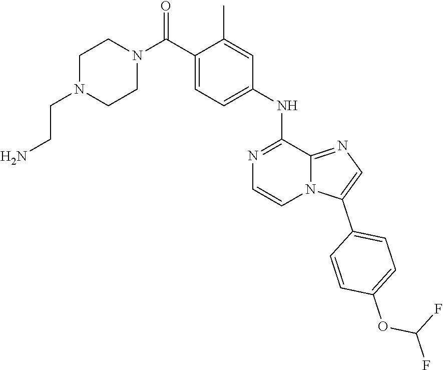 [SMILES: Cc1cc(Nc2nccn3c(-c4ccc(OC(F)F)cc4)cnc23)ccc1C(=O)N1CCN(CCN)CC1] | 522.2 | Intermediate 7 and 2- (piperazin-1- yl)ethanamine |
| REF 82 | 2-chloro-4-[[3-[4- (difluoromethoxy)- 2,3-difluoro- phenyl]imidazo[1,2- a]pyrazin-8- yl]amino]-N-(4- piperidylmethyl) benzamide; 2,2,2- trifluoroacetic acid | 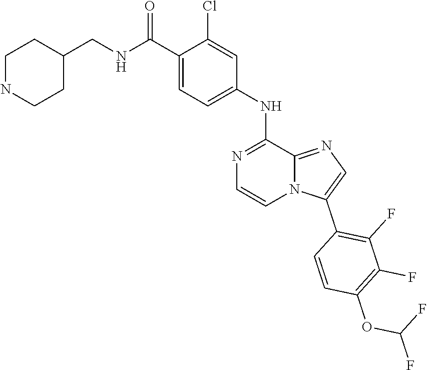 [SMILES: O=C(NCC1CCNCC1)c1ccc(Nc2nccn3c(-c4ccc(OC(F)F)c(F)c4F)cnc23)cc1Cl] | 563.1 | Intermediate 22 and piperidin-4- ylmethanamine |
| REF 83 | 2-chloro-4-[[3-(2,3- difluoro-4-methoxy- phenyl)imidazo[1,2- a]pyrazin-8- yl]amino]-N-methyl- N-(2-piperazin-1- ylethyl)benzamide; 2,2,2-trifluoroacetic acid | 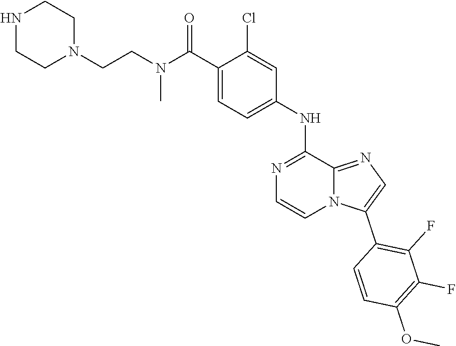 [SMILES: COc1ccc(-c2cnc3c(Nc4ccc(C(=O)N(C)CCN5CCNCC5)c(Cl)c4)nccn23)c(F)c1F] | 556.2 | Intermediate 20 and tert-butyl 4-(2- (methyalmino)ethyl) piperazine-1- carboxylate |
| REF 84 | 4-[[3-[4-(di- fluoromethoxy)phe- nyl]imidazo[1,2- a]pyrazin-8- yl]amino]-N,2- dimethyl-N-(2- piperazin-1- ylethyl)benzamide | 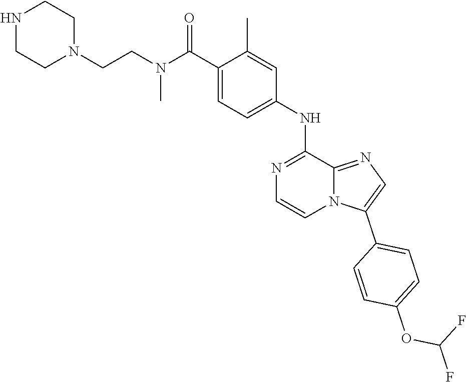 [SMILES: Cc1cc(Nc2nccn3c(-c4ccc(OC(F)F)cc4)cnc23)ccc1C(=O)N(C)CCN1CCNCC1] | 536.3 | Intermediate 7 and tert-butyl 4-(2- (methylamino)ethyl) piperazine-1- carboxylate |
| REF 85 | tert-butyl 4-[2-[[4- [[3-[4-(di- fluoromethoxy)phe- nyl]imidazo[1,2- a]pyrazin-8- yl]amino]-2-methyl- benzoyl]ami- no]ethyl] piperazine-1- carboxylate | 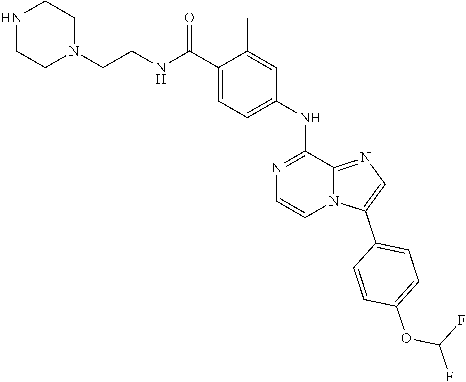 [SMILES: Cc1cc(Nc2nccn3c(-c4ccc(OC(F)F)cc4)cnc23)ccc1C(=O)NCCN1CCNCC1] | 522.2 | Intermediate 7 and tert-butyl 4-(2- aminoethyl)piper- azine-1- carboxylate |
| REF 86 | tert-butyl 3-[[[4-[[3- [4-(di- fluoromethoxy)phe- nyl]imidazo[1,2- a]pyrazin-8- yl]amino]-2-methyl- benzoyl]amino] methyl]pyrroli- dine-1- carboxylate | 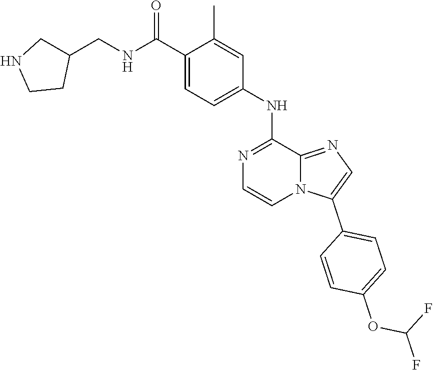 [SMILES: Cc1cc(Nc2nccn3c(-c4ccc(OC(F)F)cc4)cnc23)ccc1C(=O)NCC1CCNC1] | 492.3 | Intermediate 7 and tert-butyl 3-(amino- methyl)pyrroli- dine-1-carboxylate |
| 6 | 2-[3-chloro-4-[8-[3- chloro-4-[4- [(dimethyl- amino)meth- yl]piperidine-1-car- bonyl]anilino]imi- dazo[1,2-a]pyrazin- 3-yl]-2-fluoro- phenoxy] acetonitrile; formic acid | 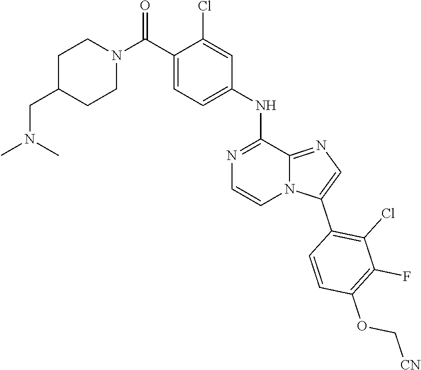 [SMILES: CN(C)CC1CCN(C(=O)c2ccc(Nc3nccn4c(-c5ccc(OCC#N)c(F)c5Cl)cnc34)cc2Cl)CC1] | 596.2 | Intermediate 80 and N,N-dimethyl-1-(4- piperidyl) methanamine |
| REF 87 | 2-[3-chloro-4-[8-[3- chloro-4-[4-[2- (dimethyl- amino)ethyl] piperazine-1- carbonyl] anilino]imidazo[1,2- a]pyrazin-3-yl]-2- fluoro- phenoxy]acetonitrile | 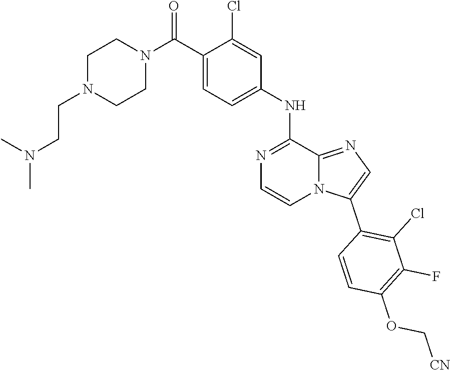 [SMILES: CN(C)CCN1CCN(C(=O)c2ccc(Nc3nccn4c(-c5ccc(OCC#N)c(F)c5Cl)cnc34)cc2Cl)CC1] | 611.2 | Intermediate 80 and 1-(2- dimethylaminoethyl) piperazine |
| REF 88 | [4-(aminomethyl)-1- piperidyl]-[4-[[3-(2- chloro-5-fluoro-4- methoxy- phenyl)imidazo[1,2- a]pyrazin-8- yl]amino]-2-methyl- phenyl]methanone hydrochloride | 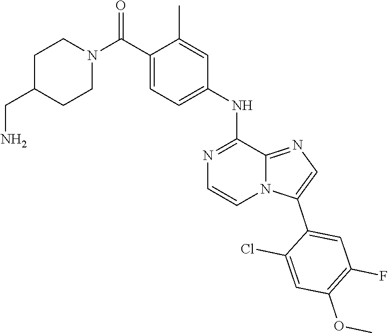 [SMILES: COc1cc(Cl)c(-c2cnc3c(Nc4ccc(C(=O)N5CCC(CN)CC5)c(C)c4)nccn23)cc1F] | 523.2 | Intermediate 26 and 4-(tert-butoxy carbonyl aminomethyl piperidine |
| REF 89 | N-(2-(2- aminoethoxy)ethyl)- 4-((3-(4-methoxy- phenyl)imida- zo[1,2-a]pyrazin-8- yl)amino)-2- methylbenzamide hydrochloride | 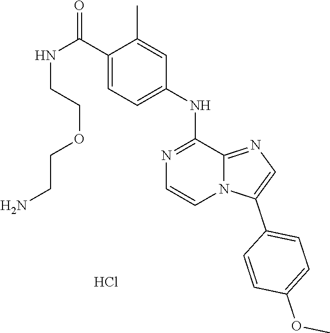 [SMILES: COc1ccc(-c2cnc3c(Nc4ccc(C(=O)NCCOCCN)c(C)c4)nccn23)cc1 ; Cl] | 461.2 | Intermediate 4 and tert-butyl (2-(2- aminoethoxy)ethyl) carbamate |
| REF 90 | N-(2-(2- aminoethoxy)ethyl)- 4-((3-(4-methoxy- phenyl)imida- zo[1,2-a]pyrazin-8- yl)amino)benzamide hydrochloride | 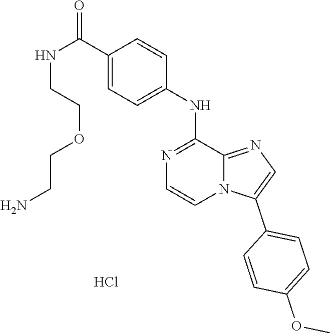 [SMILES: COc1ccc(-c2cnc3c(Nc4ccc(C(=O)NCCOCCN)cc4)nccn23)cc1 ; Cl] | 447.2 | Intermediate 17 and tert-btuyl (2- (2- aminoethoxy)ethyl) carbamate |
| REF 91 | 4-((3-(4-methoxy- phenyl)imida- zo[1,2-a]pyrazin-8- yl)methyl)-2-methyl- N-(2-(1- methylpiperidin-4- yl)ethyl)benzamide | 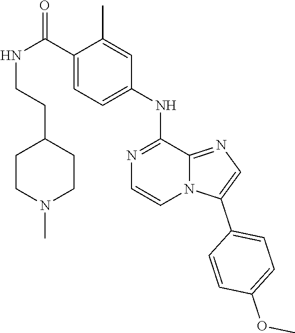 [SMILES: COc1ccc(-c2cnc3c(Nc4ccc(C(=O)NCCC5CCN(C)CC5)c(C)c4)nccn23)cc1] | 497 [M − H]⁻ | Intermediate 4 and 2- (1-methylpiperidin-4- yl)ethanamine |
| REF 92 | 4-((3-(4-methoxy- phenyl)imida- zo[1,2-a]pyrazin-8- yl)methyl)-2-methyl- N-(2-(4- methylpiperazin-1- yl)ethyl)benzamide | 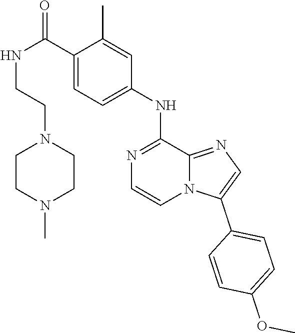 [SMILES: COc1ccc(-c2cnc3c(Nc4ccc(C(=O)NCCN5CCN(C)CC5)c(C)c4)nccn23)cc1] | 500 | Intermediate 4 and 2- (4-methylpiperazin- 1-yl)ethanamine hydrochloride |
| REF 93 | 4-((3-(4-(di- fluoromethoxy)phe- nyl)imidazo[1,2- a]pyrazin-8- yl)amino)-N-(2- (dimethylamino)eth- yl)-N,2- dimethylbenzamide | 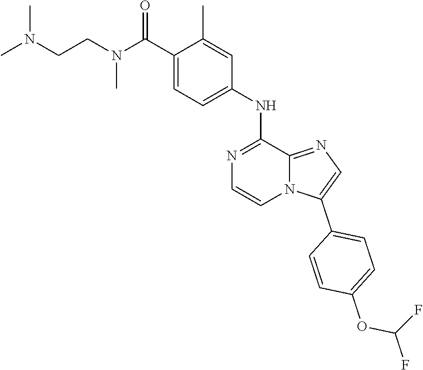 [SMILES: Cc1cc(Nc2nccn3c(-c4ccc(OC(F)F)cc4)cnc23)ccc1C(=O)N(C)CCN(C)C] | 495 | Intermediate 7 and N1,N1,N2- trimethylethane-1,2- diamine |
| REF 94 | N-(3-aminopropyl)- 4-((3-(4-methoxy- phenyl)imida- zo[1,2-a]pyrazin-8- yl)amino)-2- methylbenzamide hydrochloride | 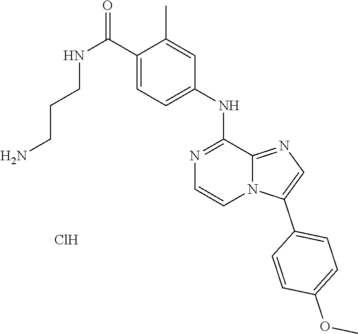 [SMILES: COc1ccc(-c2cnc3c(Nc4ccc(C(=O)NCCCN)c(C)c4)nccn23)cc1 ; Cl] | 431 | Intermediate 4 and tert-butyl (3- aminopropyl)carba- mate |
| REF 95 | 4-[[3-(4-methoxy- phenyl)imida- zo[1,2-a]pyrazin-8- yl]amino]-2-methyl- N-[(1- methylpiperidin-4- yl)methyl]benzamide | 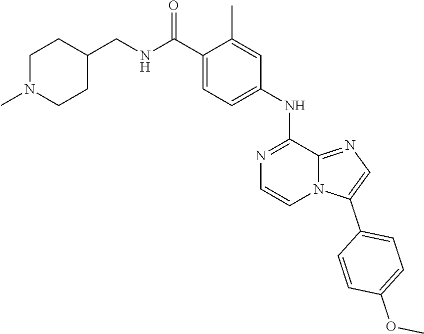 | 483.3 [M − H]⁻ | Intermediate 4 and N,1- dimethylpiperidin-4- amine |
| REF 96 | 4-((3-(4-(di- fluoromethoxy)phe- nyl)imidazo[1,2- a]pyrazin-8- yl)amino)-N-methyl- 2- (trifluoromethyl)ben- zamide | 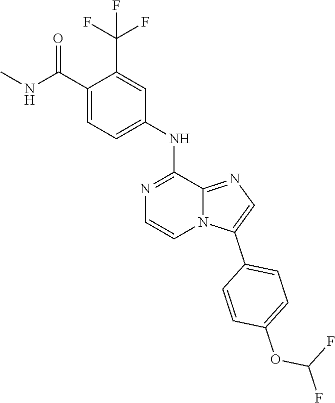 [SMILES: CNC(=O)c1ccc(Nc2nccn3c(-c4ccc(OC(F)F)cc4)cnc23)cc1C(F)(F)F] | 448.3 | Intermediate 18 and methanamine hydrochloride |
| REF 97 | 4-((3-(4-(di- fluoromethoxy)phe- nyl)imidazo[1,2- a]pyrazin-8- yl)amino)-N-methyl- 2-nitrobenzamide | 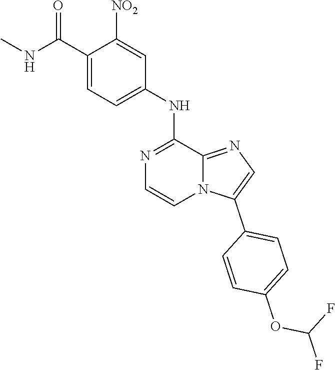 [SMILES: CNC(=O)c1ccc(Nc2nccn3c(-c4ccc(OC(F)F)cc4)cnc23)cc1[N+](=O)[O-]] | 455.3 | Intermediate 19 and methanamine hydrochloride |
| REF 98 | N-(2-(2-(2- aminoethoxy)ethoxy) ethyl)-4-((3-(4-(di- fluoromethoxy)phe- nyl)imidazo[1,2- a]pyrazin-8- yl)amino)-2- methylbenzamide | 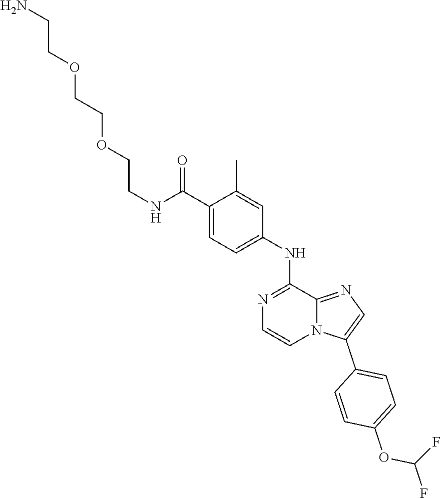 [SMILES: Cc1cc(Nc2nccn3c(-c4ccc(OC(F)F)cc4)cnc23)ccc1C(=O)NCCOCCOCCN] | 539.5 [M − H]⁻ | Intermediate 7 and tert-butyl (2-(2-(2- aminoethoxy)ethoxy) ethyl)carbamate |
| REF 99 | N-(2-(2-(2-(2- aminoethoxy)ethoxy) ethoxy)ethyl)- 4-((3-(4-(difluoro- methoxy)phe- nyl)imidazo[1,2- a]pyrazin-8- yl)amino)-2- methylbenzamide | 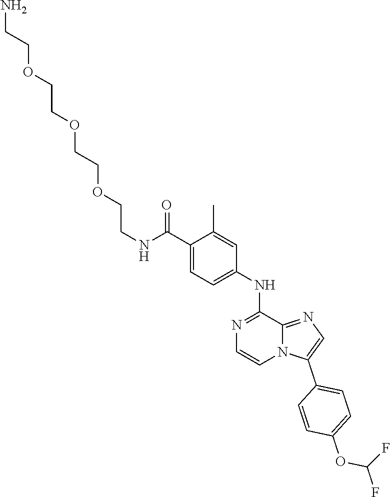 [SMILES: Cc1cc(Nc2nccn3c(-c4ccc(OC(F)F)cc4)cnc23)ccc1C(=O)NCCOCCOCCOCCN] | 583.5 [M − H]⁻ | Intermediate 7 and tert-butyl (2-(2-(2-(2- aminoethoxy)ethoxy) ethoxy)ethyl)carba- mate |
| REF 100 | 4-[[3-(2,3-difluoro- 4-methoxy- phenyl)imidazo[1,2- a]pyrazin-8- yl]amino]-2-methyl- N-(4-piperi- dylmethyl)ben- zamide | 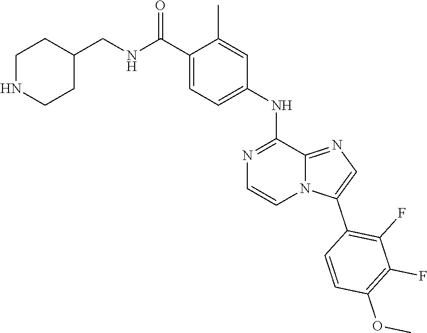 [SMILES: COc1ccc(-c2cnc3c(Nc4ccc(C(=O)NCC5CCNCC5)c(C)c4)nccn23)c(F)c1F] | 507.2 | Intermediate 28 and tetrahydropyridtert- butyl 4- (aminomethyl)piperi- dine-1-carboxylate |
| REF 101 | 4-((3-(2,3-difluoro- 4-methoxy- phenyl)imida- zo[1,2-a]pyrazin-8- yl)amino)-2-methyl- N-(2-(piperazin-1- yl)ethyl)benzamide hydrochloride | 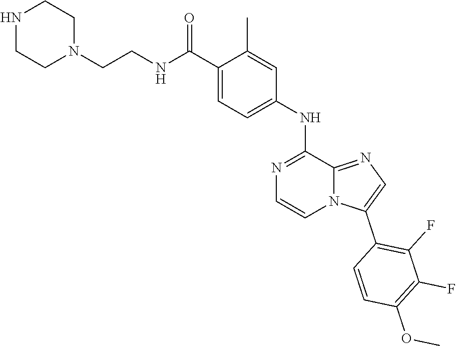 [SMILES: COc1ccc(-c2cnc3c(Nc4ccc(C(=O)NCCN5CCNCC5)c(C)c4)nccn23)c(F)c1F] | 522.2 | Intermediate 28 and 2-(piperazin-1- yl)ethanamine |
| REF 102 | 4-[[3-(2,3-difluoro- 4-methoxy- phenyl)imidazo[1,2- a]pyrazin-8- yl]amino]-2-methyl- N-(pyrrolidin-3- ylmethyl)benzamide | 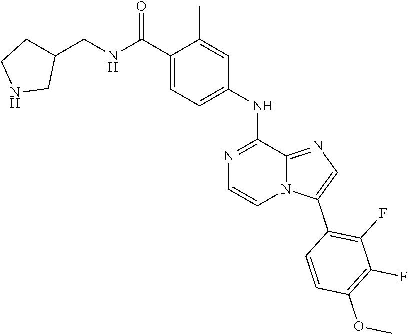 [SMILES: COc1ccc(-c2cnc3c(Nc4ccc(C(=O)NCC5CCNC5)c(C)c4)nccn23)c(F)c1F] | 493.2 | Intermediate 28 and tert-butyl 3- (aminomethyl)pyrroli- dine-1-carboxylate |
| REF 103 | 2-chloro-4-[[3-(2,3- difluoro-4-methoxy- phenyl)imidazo[1,2- a]pyrazin-8- yl]amino]-N-(4- piperidylmethyl)ben- zamide | 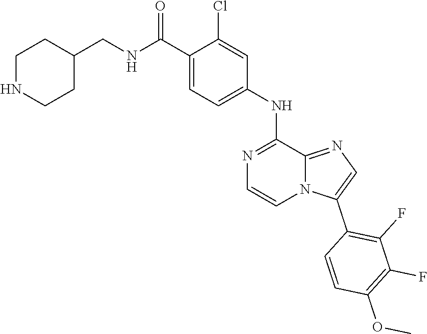 [SMILES: COc1ccc(-c2cnc3c(Nc4ccc(C(=O)NCC5CCNCC5)c(Cl)c4)nccn23)c(F)c1F] | 527.1 | Intermediate 20 and tert-butyl 4-(amino- methyl)piperidine- 1-carboxylate |
| REF 104 | 2-chloro-4-[[3-(2,3- difluoro-4-methoxy- phenyl)imidazo[1,2- a]pyrazin-8- yl]amino]-N-(2- piperazin-1- ylethyl)benzamide | 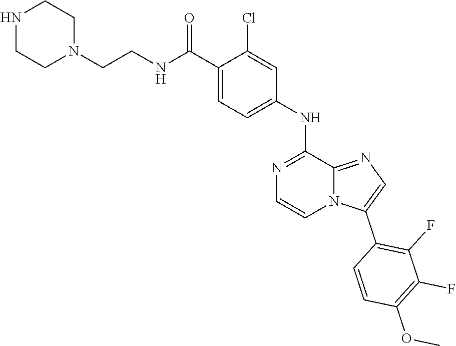 [SMILES: COc1ccc(-c2cnc3c(Nc4ccc(C(=O)NCCN5CCNCC5)c(Cl)c4)nccn23)c(F)c1F] | 542.1 | Intermediate 20 and tert-butyl 4-(2- aminoethyl)pipera- zine-1-carboxylate |
| REF 105 | 2-chloro-4-[[3-(2,3- difluoro-4-methoxy- phenyl)imidazo[1,2- a]pyrazin-8- yl]amino]-N- (pyrrolidin-3- ylmethyl)benzamide | 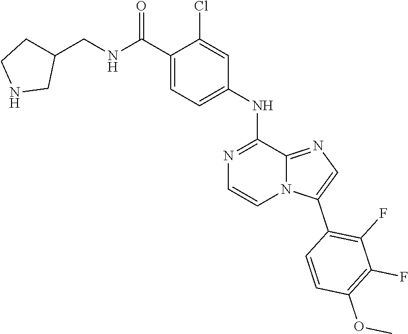 [SMILES: COc1ccc(-c2cnc3c(Nc4ccc(C(=O)NCC5CCNC5)c(Cl)c4)nccn23)c(F)c1F] | 513.1 | Intermediate 20 and tert-butyl 3- (aminomethyl)pyrroli- dine-1-carboxylate |
| 7 | (4-(2-amino- ethyl)piperidin- 1-yl)(2-chloro-4- ((3-(2,3-difluoro-4- methoxyphenyl)imi- dazo[1,2-a]pyrazin- 8-yl)amino)phenyl) methanone | 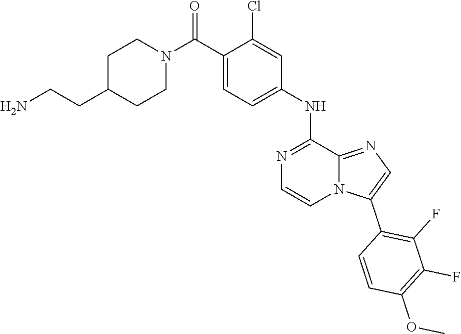 [SMILES: COc1ccc(-c2cnc3c(Nc4ccc(C(=O)N5CCC(CCN)CC5)c(Cl)c4)nccn23)c(F)c1F] | 541.1 | Intermediate 20 and tert-butyl (2- (piperidin-4- yl)ethyl)carbamate |
| 8 | (4- (aminomethyl)piperi- din-1-yl)(2-chloro-4- ((3-(2,3-difluoro-4- methoxyphenyl)imi- dazo[1,2-a]pyrazin- 8-yl)amino)phenyl) methanone | 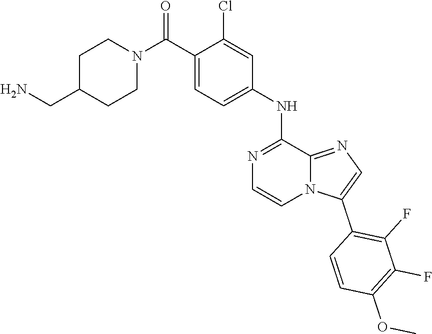 [SMILES: COc1ccc(-c2cnc3c(Nc4ccc(C(=O)N5CCC(CN)CC5)c(Cl)c4)nccn23)c(F)c1F] | 527.1 | Intermediate 20 and piperidin-4- ylmethanamine |
| REF 106 | 4-((3-(2,3-difluoro- 4-methoxy- phenyl)imida- zo[1,2-a]pyrazin-8- yl)amino)-N,2- dimethyl-N-(2- (piperazin-1- yl)ethyl)benzamide | 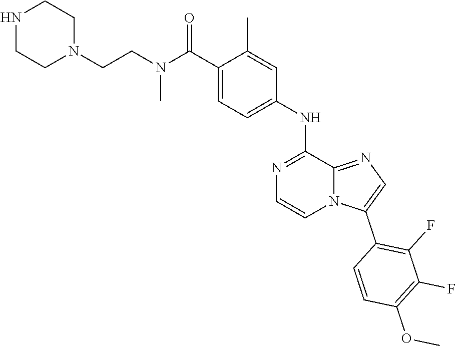 [SMILES: COc1ccc(-c2cnc3c(Nc4ccc(C(=O)N(C)CCN5CCNCC5)c(C)c4)nccn23)c(F)c1F] | 536.2 | Intermediate 28 and tert-butyl 4-(2- (methylamino)ethyl) piperazine-1- carboxylate |
| REF 107 | (4-((3-(2,3-difluoro- 4-methoxy- phenyl)imida- zo[1,2-a]pyrazin-8- yl)amino)-2- methylphenyl)(4-(2- (dimethylami- no)ethyl)piperazin- 1-yl)methanone | 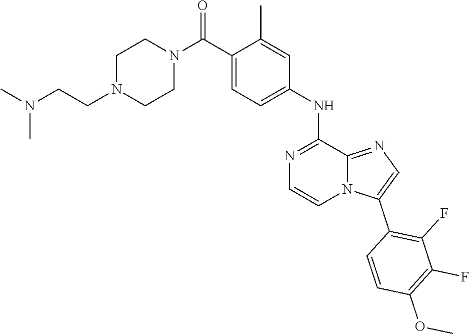 [SMILES: COc1ccc(-c2cnc3c(Nc4ccc(C(=O)N5CCN(CCN(C)C)CC5)c(C)c4)nccn23)c(F)c1F] | 550.2 | Intermediate 28 and N,N-dimethyl-2- (piperazin-1- yl)ethanamine |
| REF 108 | (4- (aminomethyl)piperi- din-1-yl)(4-((3-(2,3- difluoro-4- methoxy- phenyl)imida- zo[1,2-a]pyrazin-8- yl)amino)-2- methylphenyl)metha- none | 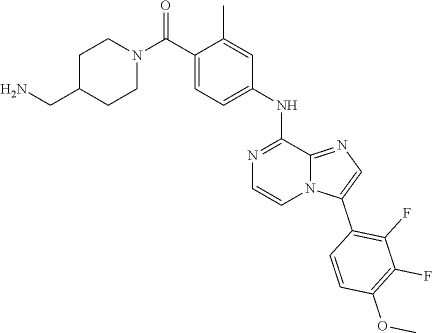 [SMILES: COc1ccc(-c2cnc3c(Nc4ccc(C(=O)N5CCC(CN)CC5)c(C)c4)nccn23)c(F)c1F] | 507.2 | Intermediate 28 and piperidin-4- ylmethanamine |
| REF 109 | N-(2-(2- aminoethoxy)ethyl)- 2-chloro-4-((3-(2,3- difluoro-4-methoxy- phenyl)imida- zo[1,2-a]pyrazin-8- yl)amino)benzamide | 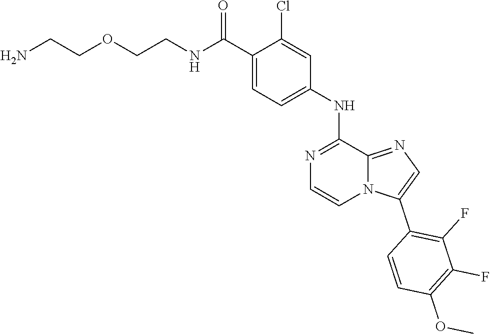 [SMILES: COc1ccc(-c2cnc3c(Nc4ccc(C(=O)NCCOCCN)c(Cl)c4)nccn23)c(F)c1F] | 517.1 | Intermediate 20 and piperidin-4- ylmethanamine |
| REF 110 | (4-((3-(2,3-difluoro- 4-methoxy- phenyl)imida- zo[1,2-a]pyrazin-8- yl)amino)-2- methylphenyl)(4- ((dimethylamino) methyl)piperidin-1- yl)methanone | 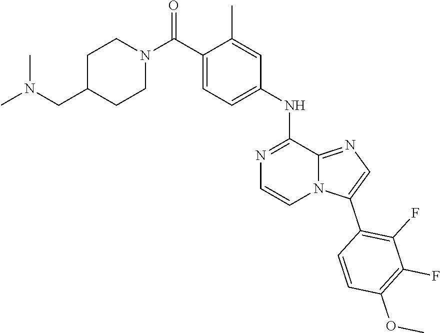 [SMILES: COc1ccc(-c2cnc3c(Nc4ccc(C(=O)N5CCC(CN(C)C)CC5)c(C)c4)nccn23)c(F)c1F] | 535.2 | Intermediate 28 and N,N-Dimethyl-1- (piperidin-4- yl)methanamine dihydrochloride |
| REF 111 | 4-((3-(2,3-difluoro- 4-methoxy- phenyl)imida- zo[1,2-a]pyrazin-8- yl)amino)-2-methyl- N-((1- methylpiperidin-4- yl)methyl)benzamide | 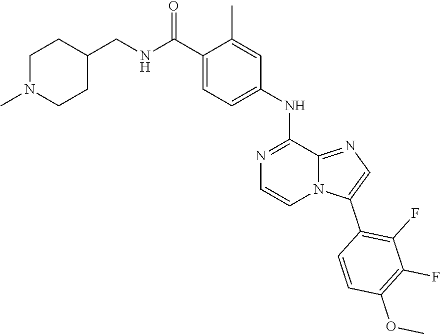 [SMILES: COc1ccc(-c2cnc3c(Nc4ccc(C(=O)NCC5CCN(C)CC5)c(C)c4)nccn23)c(F)c1F] | 521.2 | Intermediate 28 and (1-methylpiperidin-4- yl)methanamine |
| 9 | aziridin-1-yl(2- chloro-4-((3-(2,3- difluoro-4- methoxy- phenyl)imida- zo[1,2-a]pyrazin-8- yl)amino)phenyl) methanone | 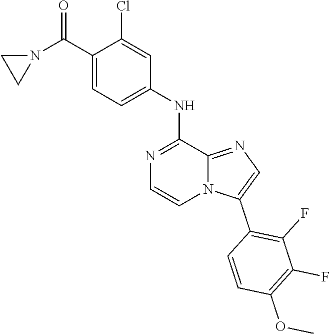 [SMILES: COc1ccc(-c2cnc3c(Nc4ccc(C(=O)N5CC5)c(Cl)c4)nccn23)c(F)c1F] | 456.0 | Intermediate 20 and 2-chloroethanamine hydrochloride |
| REF 112 | 4-((3-(2,3-difluoro- 4-methoxy- phenyl)imida- zo[1,2-a]pyrazin-8- yl)amino)-N,2- dimethyl-N-(2- (piperidin-4- yl)ethyl)benzamide | 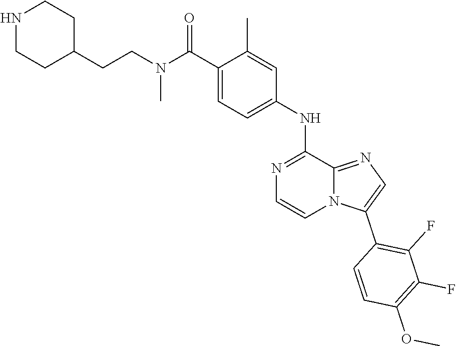 [SMILES: COc1ccc(-c2cnc3c(Nc4ccc(C(=O)N(C)CCC5CCNCC5)c(C)c4)nccn23)c(F)c1F] | 535.2 | Intermediate 28 and tert-butyl 4-[2- (methylamino)ethyl] piperidine-1- carboxylate |
| REF 113 | N-(3-aminopropyl)- 4-((3-(2,3-difluoro- 4-methoxy- phenyl)imida- zo[1,2-a]pyrazin-8- yl)amino)-2- methylbenzamide | 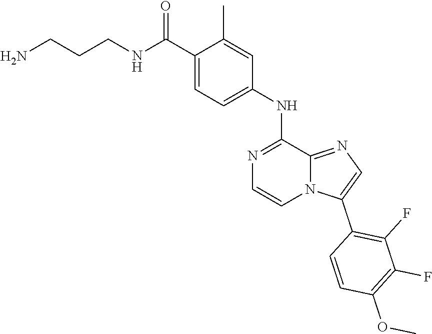 [SMILES: COc1ccc(-c2cnc3c(Nc4ccc(C(=O)NCCCN)c(C)c4)nccn23)c(F)c1F] | 467.1 | Intermediate 28 and tert-butyl (3- aminopropyl)carba- mate |
| REF 114 | 4-((3-(2,3-difluoro- 4-methoxy- phenyl)imida- zo[1,2-a]pyrazin-8- yl)amino)-2-methyl- N-(3-(3- oxopiperazin-1- yl)propyl)benzamide | 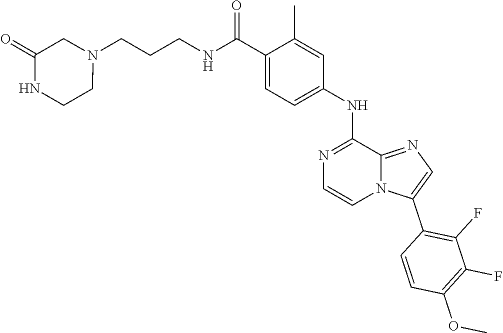 [SMILES: COc1ccc(-c2cnc3c(Nc4ccc(C(=O)NCCCN5CCNC(=O)C5)c(C)c4)nccn23)c(F)c1F] | 550.2 | Intermediate 28 and Intermediate 57-P1 |
| REF 115 | 4-((3-(2,3-difluoro- 4-methoxy- phenyl)imida- zo[1,2-a]pyrazin-8- yl)amino)-2-methyl- N-(3-(4-methyl-3- oxopiperazin-1- yl)propyl)benzamide | 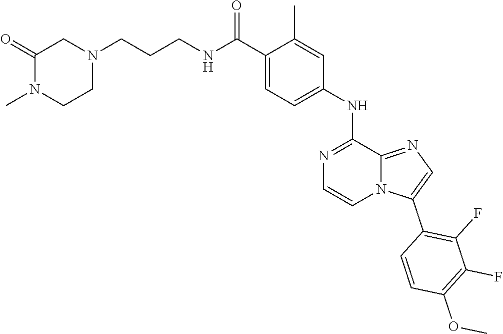 [SMILES: COc1ccc(-c2cnc3c(Nc4ccc(C(=O)NCCCN5CCN(C)C(=O)C5)c(C)c4)nccn23)c(F)c1F] | 564.2 | Intermediate 28 and Intermediate 58 |
| REF 116 | 4-((3-(2,3-difluoro- 4-methoxy- phenyl)imida- zo[1,2-a]pyrazin-8- yl)amino)-2-methyl- N-(3-(piperazin-1- yl)propyl)benzamide | 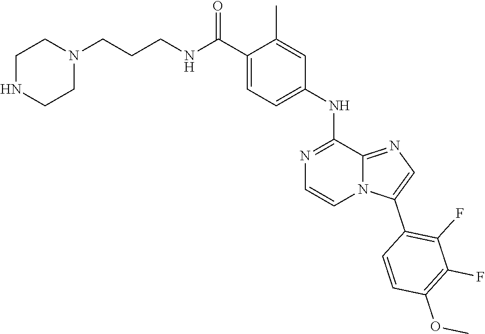 [SMILES: COc1ccc(-c2cnc3c(Nc4ccc(C(=O)NCCCN5CCNCC5)c(C)c4)nccn23)c(F)c1F] | 536.2 | Intermediate 28 and tert-butyl 4-(3- aminopropyl)pipera- zine-1-carboxylate |
| REF 117 | 4-((3-(2,3-difluoro- 4-methoxy- phenyl)imida- zo[1,2-a]pyrazin-8- yl)amino)-2-methyl- N-(3-(piperazin-1- ylsulfonyl)propyl) benzamide | 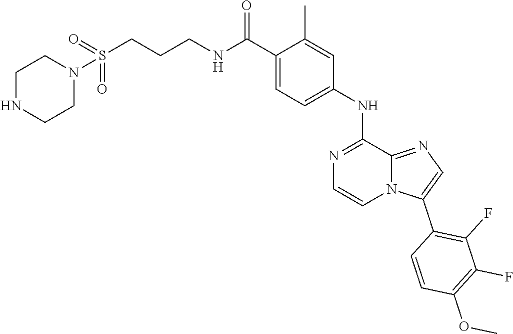 [SMILES: COc1ccc(-c2cnc3c(Nc4ccc(C(=O)NCCCS(=O)(=O)N5CCNCC5)c(C)c4)nccn23)c(F)c1F] | 600.2 | Intermediate 28 and tert-butyl 4-((3- aminopropyl)sulfonyl) piperazine-1- carboxylate |
| REF 118 | 4-((3-(2,3-difluoro- 4-methoxy- phenyl)imida- zo[1,2-a]pyrazin-8- yl)amino)-2-methyl- N-(3-(2- oxopiperazin-1- yl)propyl)benzamide | 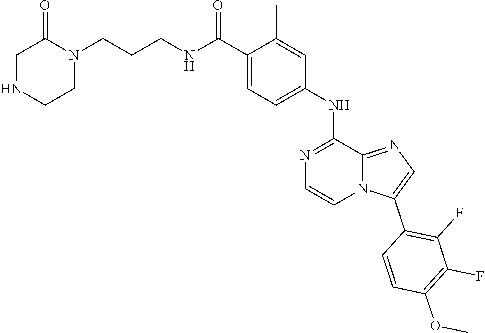 [SMILES: COc1ccc(-c2cnc3c(Nc4ccc(C(=O)NCCCN5CCNCC5=O)c(C)c4)nccn23)c(F)c1F] | 550.2 | Intermediate 28 and Intermediate 59 |
| REF 119 | 4-((3-(3-fluoro-4- methoxyphenyl)imi- dazo[1,2-a]pyra- zin-8-yl)amino)- 2-methyl-N-((tetra- hydrofuran-3- yl)methyl)benz- amide | 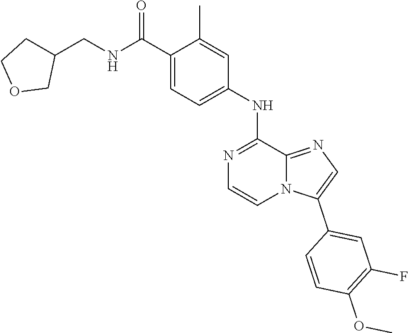 [SMILES: COc1ccc(-c2cnc3c(Nc4ccc(C(=O)NCC5CCOC5)c(C)c4)nccn23)cc1F] | 476.2 | Intermediate 6 and (tetrahydrofuran-3- yl)methanamine |
| REF 120 | 4-((3-(3-fluoro-4- methoxyphenyl)imi- dazo[1,2-a]pyra- zin-8-yl)amino)- N,2-dimethyl-N- ((tetrahydrofuran-3- yl)methyl)benz- amide | 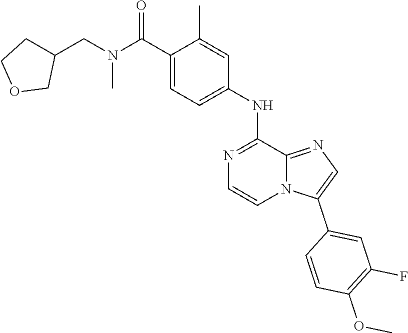 [SMILES: COc1ccc(-c2cnc3c(Nc4ccc(C(=O)N(C)CC5CCOC5)c(C)c4)nccn23)cc1F] | 490.2 | Intermediate 6 and N- methyl-1- (tetrahydrofuran-3- yl)methanamine |
| REF 121 | 4-((3-(3-fluoro-4- methoxy- phenyl)imida- zo[1,2-a]pyrazin-8- yl)amino)-2-methyl- N-((tetrahydro-2H- pyran-3-yl) methyl)benzamide | 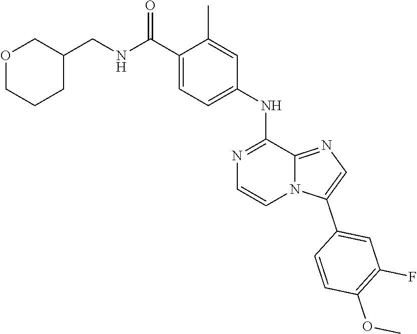 [SMILES: COc1ccc(-c2cnc3c(Nc4ccc(C(=O)NCC5CCCOC5)c(C)c4)nccn23)cc1F] | 490.2 | Intermediate 6 and (tetrahydrofuran-3- yl)methanamine |
| REF 122 | 4-((3-(3-fluoro-4- methoxyphenyl)imi- dazo[1,2-a]pyra- zin-8-yl)amino)- N,2-dimethyl- N-(pyridin-4- ylmethyl)benzamide | 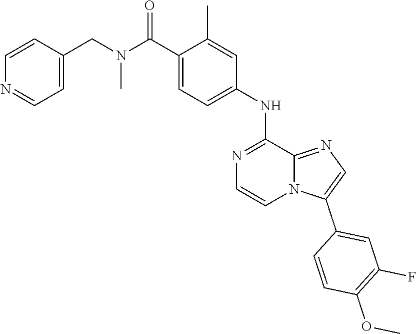 [SMILES: COc1ccc(-c2cnc3c(Nc4ccc(C(=O)N(C)Cc5ccncc5)c(C)c4)nccn23)cc1F] | 497.2 | Intermediate 6 and N- methyl-1-(pyridin-4- yl)methanamine |
| 10 | 2-(4-(8-((3-chloro-4- (4-(2-(dimethyl- amino)ethyl) piperazine-1- carbonyl)phe- nyl)ami- no)imidazo[1,2- a]pyrazin-3-yl)-2,3- difluorophenoxy) acetonitrile | 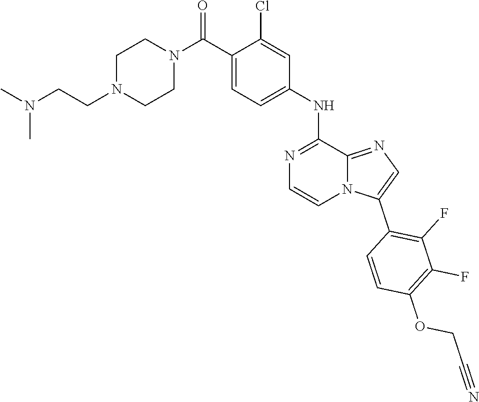 [SMILES: CN(C)CCN1CCN(C(=O)c2ccc(Nc3nccn4c(-c5ccc(OCC#N)c(F)c5F)cnc34)cc2Cl)CC1] | 595.2 | Intermediate 29 and N,N-dimethyl-2- (piperazin-1- yl)ethanamine |
| REF 123 | 2-chloro-4-((3-(4- (cyanomethoxy)-2,3- difluorophenyl)imi- dazo[1,2-a]pyra- zin-8- yl)amino)-N-methyl- N-(2-(piperazin-1- yl)ethyl)benzamide | 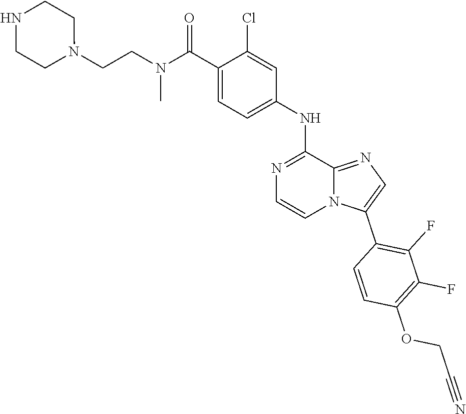 [SMILES: CN(CCN1CCNCC1)C(=O)c1ccc(Nc2nccn3c(-c4ccc(OCC#N)c(F)c4F)cnc23)cc1Cl] | 581.2 | Intermediate 29 and tert-butyl 4-(2- (methylamino)ethyl) piperazine-1- carboxylate |
| REF 124 | 2-chloro-4-((3-(4- (cyanomethoxy)-2,3- difluorophenyl)imi- dazo[1,2-a]pyra- zin-8- yl)amino)-N-(2- (pyridin-4- yl)ethyl)benzamide | 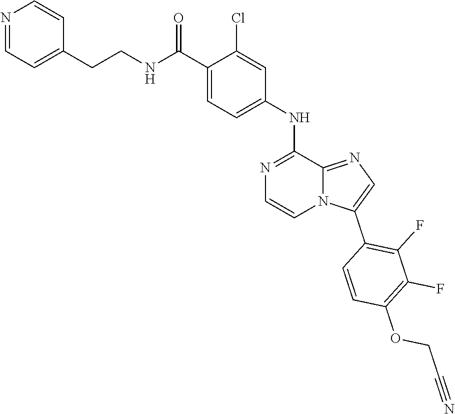 [SMILES: N#CCOc1ccc(-c2cnc3c(Nc4ccc(C(=O)NCCc5ccncc5)c(Cl)c4)nccn23)c(F)c1F] | 560.1 | Intermediate 29 and 2-(pyridin-4- yl)ethanamine |
| REF 125 | 2-chloro-4-[[3-[4- (cyanomethoxy)-2,3- difluoro- phenyl]imidazo[1,2- a]pyrazin-8- yl]amino]-N-[3-(4- pyridyl)propyl]ben- zamide; 2,2,2- trifluoroacetic acid | 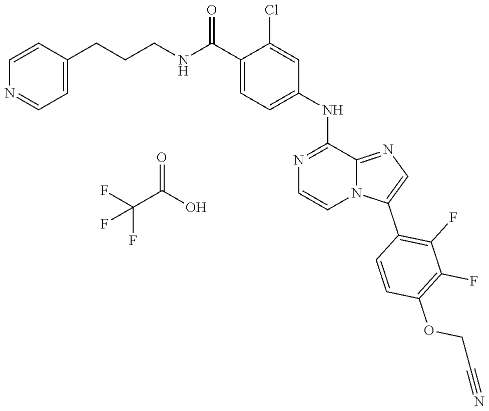 [SMILES: N#CCOc1ccc(-c2cnc3c(Nc4ccc(C(=O)NCCCc5ccncc5)c(Cl)c4)nccn23)c(F)c1F ; O=C(O)C(F)(F)F] | 574.1 | Intermediate 29 and 3-(pyridin-4- yl)propan-1-amine |
| 11 | 2-[4-[8-[3-chloro-4- [4-(1H-imidazol-5- yl)piperidine-1- carbonyl]anilino]imi- dazo[1,2-a]pyrazin- 3-yl]-2,3-difluoro- phenoxy]acetontrile; 2,2,2-trifluoroacetic acid | 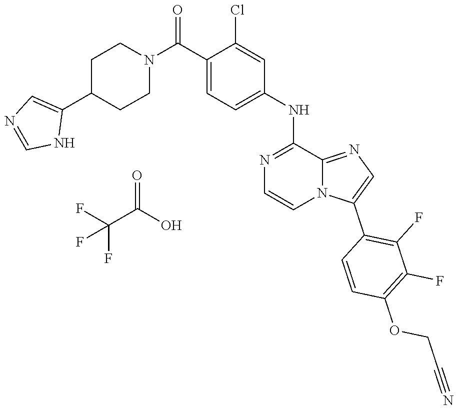 [SMILES: N#CCOc1ccc(-c2cnc3c(Nc4ccc(C(=O)N5CCC(c6cnc[nH]6)CC5)c(Cl)c4)nccn23)c(F)c1F ; O=C(O)C(F)(F)F] | 589.1 | Intermediate 29 and 4-(1H-imidazol-5- yl)piperidine |
| REF 126 | 2-chloro-4-((3-(4- (cyanomethoxy)-2,3- difluorophenyl)imi- dazo[1,2-a]pyrazin- 8-yl)amino)-N-(3- (piperazin-1- yl)propyl)benzamide | 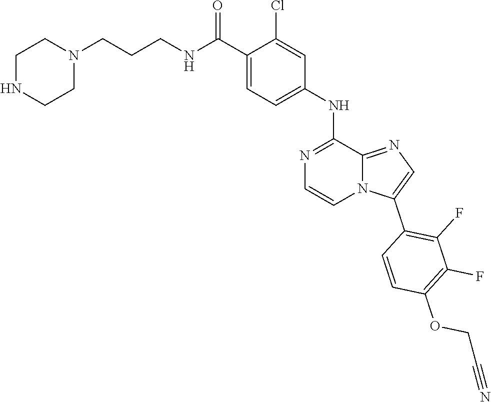 [SMILES: N#CCOc1ccc(-c2cnc3c(Nc4ccc(C(=O)NCCCN5CCNCC5)c(Cl)c4)nccn23)c(F)c1F] | 580.1 | Intermediate 29 and tert-butyl 4-(3- aminopropyl)pipera- zine-1-carboxylate |
| REF 127 | N-(2-(2-(1H- imidazol-1- yl)ethoxy)ethyl)-2- chloro-4-((3-(4- (cyanomethoxy)-2,3- difluorophenyl)imi- dazo[1,2-a]pyrazin- 8-yl)amino)benz- amide | 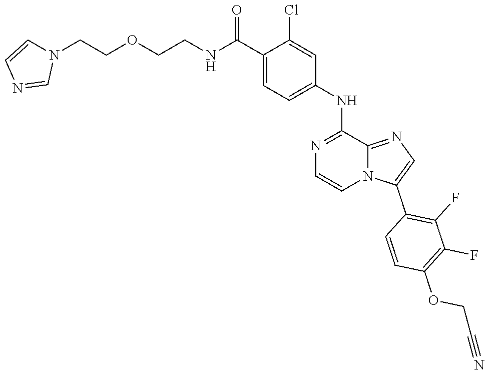 [SMILES: N#CCOc1ccc(-c2cnc3c(Nc4ccc(C(=O)NCCOCCn5ccnc5)c(Cl)c4)nccn23)c(F)c1F] | 593.1 | Intermediate 29 and 61 |
| REF 128 | 2-(4-(8-((4-(4-(2- (dimethylamino)eth- yl)piperazine-1- carbonyl)-3-ethyl- phenyl)amino)imi- dazo[1,2- a]pyrazin-3-yl)-2,3- difluorophenoxy) acetonitrile | 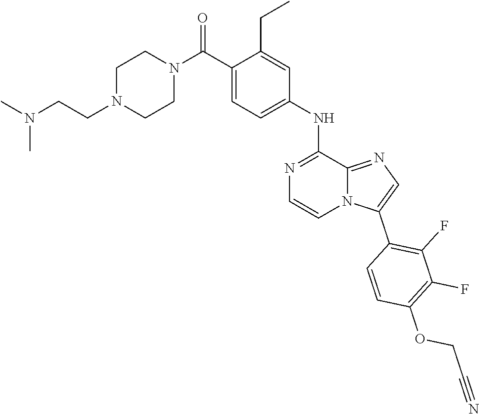 [SMILES: CCc1cc(Nc2nccn3c(-c4ccc(OCC#N)c(F)c4F)cnc23)ccc1C(=O)N1CCN(CCN(C)C)CC1] | 589.2 | Intermediate 32 and N,N-dimethyl-2- (piperazin-1- yl)ethanamine |
| 12 | 2-(4-(8-((4-(4- (aminomethyl)piperi- dine-1-carbonyl)-3- chlorophenyl)amino) imidazo[1,2- a]pyrazin-3-yl)-2,3- difluorophenoxy)ace- tonitrile | 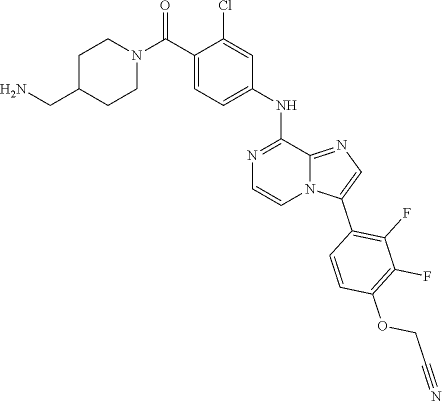 [SMILES: N#CCOc1ccc(-c2cnc3c(Nc4ccc(C(=O)N5CCC(CN)CC5)c(Cl)c4)nccn23)c(F)c1F] | 552.1 | Intermeidate 29 and tert-butyl (piperidin- 4- ylmethyl)carbamate |
| REF 129 | 2-[4-[8-[4-[4- (aminomethyl)piperi- dine-1-carbonyl]-3- methyl- anilino]imidazo[1,2- a]pyrazin-3-yl]-2,3- difluoro- phenoxy]acetonitrile; 2,2,2-trifluoroacetic acid | 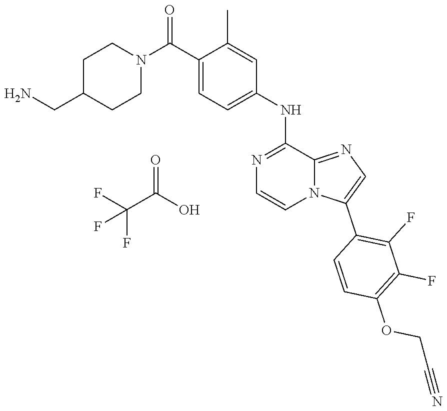 [SMILES: Cc1cc(Nc2nccn3c(-c4ccc(OCC#N)c(F)c4F)cnc23)ccc1C(=O)N1CCC(CN)CC1 ; O=C(O)C(F)(F)F] | 532.2 | Intermediate 31 and tert-butyl (piperidin-4- ylmethyl)carbamate |
| REF 130 | 2-chloro-4-[[3-[4- (cyanomethoxy)- 2,3-difluoro- phenyl]imidazo[1,2- a]pyrazin-8- yl]amino]-N-methyl- N-[2-(4- piperidyl)ethyl]benz- amide; 2,2,2- trifluoroacetic acid | 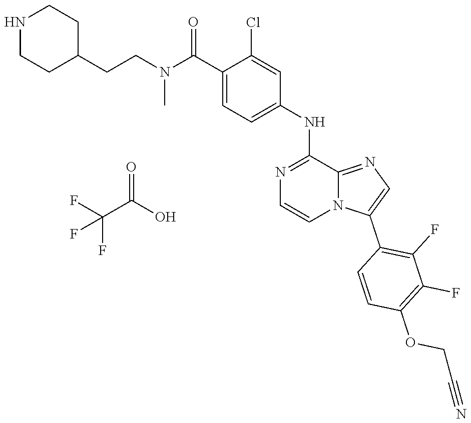 [SMILES: CN(CCC1CCNCC1)C(=O)c1ccc(Nc2nccn3c(-c4ccc(OCC#N)c(F)c4F)cnc23)cc1Cl ; O=C(O)C(F)(F)F] | 580.1 | Intermediate 29 and tert-butyl 4-(2- (methylamino)ethyl) piperidine-1- carboxylate |
| 13 | 2-[4-[8-[3-chloro-4- [4-(1H-tetrazol-5- yl)piperidine-1- carbonyl]ani- lino]imi- dazo[1,2-a]pyrazin- 3-yl]-2,3-difluoro- phenoxy]aceto- nitrile;2,2,2- trifluoroacetic acid | 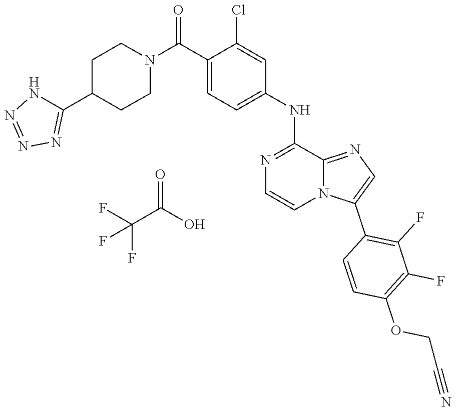 [SMILES: N#CCOc1ccc(-c2cnc3c(Nc4ccc(C(=O)N5CCC(c6nnn[nH]6)CC5)c(Cl)c4)nccn23)c(F)c1F ; O=C(O)C(F)(F)F] | 591.1 | Intermediate 29 and 4-(2H-tetrazol-5- yl)piperidine- hydrochloride |
| 14 | 2-[4-[8-[3-chloro-4- [4-[2-(dimethyl- amino)acetyl] piperazine-1-car- bonyl]anilino]imi- dazo[1,2-a]pyrazin- 3-yl]-2,3-difluoro- phenoxy]aceto- nitrile; 2,2,2- trifluoroacetic acid | 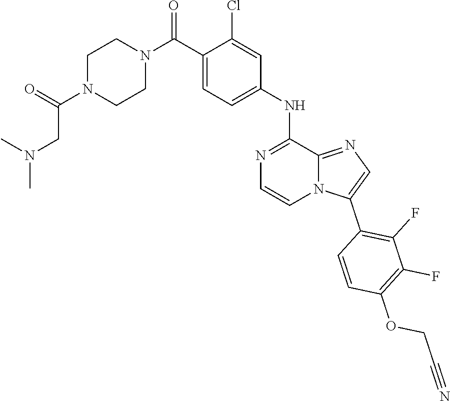 [SMILES: CN(C)CC(=O)N1CCN(C(=O)c2ccc(Nc3nccn4c(-c5ccc(OCC#N)c(F)c5F)cnc34)cc2Cl)CC1] | 609.2 | Intermediate 29 and intermediate 56 |
| REF 131 | 2-chloro-4-[[3-[4- (cyanomethoxy)- 2,3-difluoro- phenyl]imidazo[1,2- a]pyrazin-8- yl]amino]-N-(4- piperidylmethyl) benzamide | 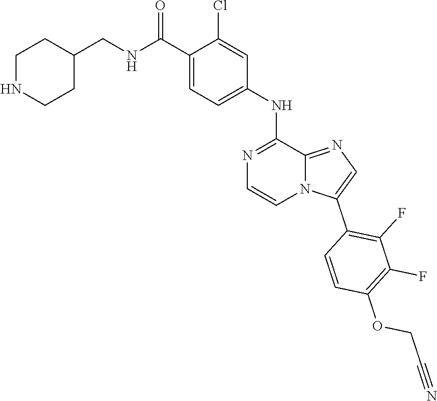 [SMILES: N#CCOc1ccc(-c2cnc3c(Nc4ccc(C(=O)NCC5CCNCC5)c(Cl)c4)nccn23)c(F)c1F] | 552.1 | Intermediate 29 and 4-piperidyl methanamine |
| REF 132 | 2-chloro-4-[[3-[3- chloro-4- (cyanomethoxy)-2- fluoro- phenyl]imidazo[1,2- a]pyrazin-8- yl]amino]-N-[2-(4- pyridyl)ethyl]benza- mide | 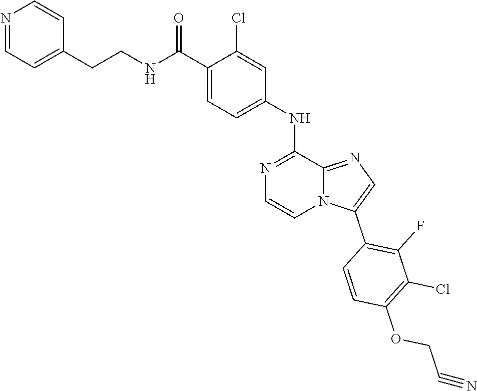 [SMILES: N#CCOc1ccc(-c2cnc3c(Nc4ccc(C(=O)NCCc5ccncc5)c(Cl)c4)nccn23)c(F)c1Cl] | 576.2 | Intermediate 35 and 2-(pyridin-4- yl)ethanamine |
| REF 133 | N-[2-(2- aminoethoxy)ethyl]- 4-[[3-[4- (cyanomethoxy)-2,3- difluoro- phenyl]imidazo[1,2- a]pyrazin-8- yl]amino]-2-ethyl- benzamide; 2,2,2- trifluoroacetic acid | 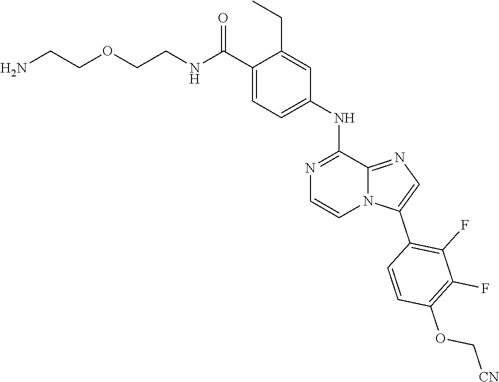 [SMILES: CCc1cc(Nc2nccn3c(-c4ccc(OCC#N)c(F)c4F)cnc23)ccc1C(=O)NCCOCCN] | 536.2 | Intermediate 32 and N-BOC-2-(2- amino-ethoxy)- ethylamine |
| REF 134 | 4-[[3-[4- (cyanomethoxy)-2,3- difluoro- phenyl]imidazo[1,2- a]pyrazin-8- yl]amino]-N-[2-[2- (dimethylamino) ethoxy]ethyl]- 2-ethyl-benzamide | 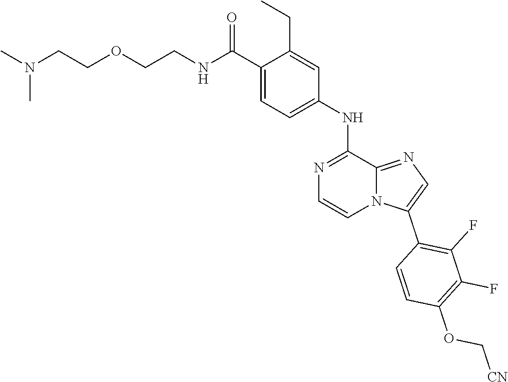 [SMILES: CCc1cc(Nc2nccn3c(-c4ccc(OCC#N)c(F)c4F)cnc23)ccc1C(=O)NCCOCCN(C)C] | 564.2 | Intermediate 32 and 2-[2- (dimethylamino) ethoxy]ethanamine |
| REF 135 | 4-[[3-[2-chloro-4- (cyanomethoxy)-3- fluoro- phenyl]imidazo[1,2- a]pyrazin-8- yl]amino]-N-[2-[2- (dimethyl- amino)ethoxy]- ethyl]-2-ethyl- benzamide | 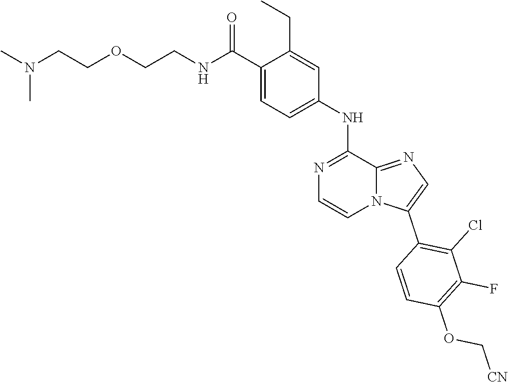 [SMILES: CCc1cc(Nc2nccn3c(-c4ccc(OCC#N)c(F)c4Cl)cnc23)ccc1C(=O)NCCOCCN(C)C] | 580.2 | Intermediate 40 and 2-[2-(dimethylamino) ethoxy]ethanamine |
| 15 | 2-[5-chloro-4-[8-[3- chloro-4-[4-[3- (hydroxymethyl)pi- perazine-1- carbonyl]piperidine- 1-carbo- nyl]anilino]imi- dazo[1,2-a]pyrazin- 3-yl]-2-fluoro- phenoxy]aceto- nitrile; 2,2,2-trifluoroacetic | 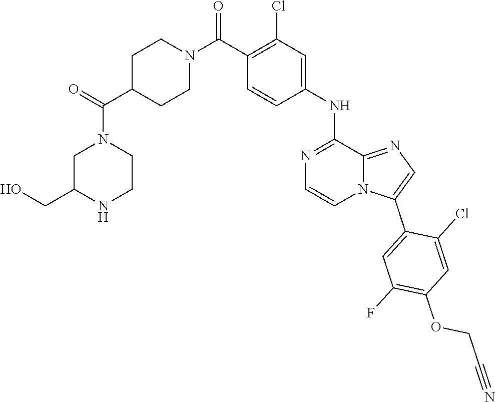 [SMILES: N#CCOc1cc(Cl)c(-c2cnc3c(Nc4ccc(C(=O)N5CCC(C(=O)N6CCNC(CO)C6)CC5)c(Cl)c4)nccn23)cc1F] | 681.1 | Intermediate 37 and 81 |
| REF 136 | 2-[5-chloro-2-fluoro- 4-[8-[4-[4-[3- (hydroxymethyl)pi- perazine-1- carbonyl]piperidine- 1-carbonyl]-3- methyl- anilino]imidazo[1,2- a]pyrazin-3- yl]phenoxy]aceto- nitrile; 2,2,2- trifluoroacetic acid | 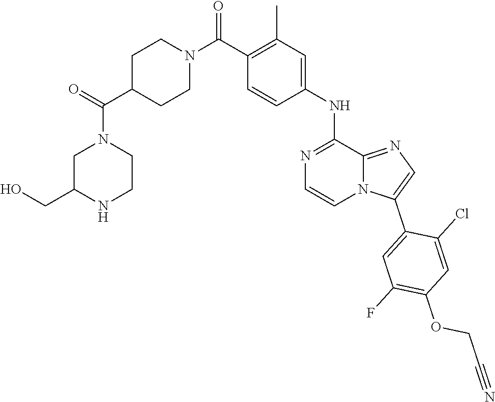 [SMILES: Cc1cc(Nc2nccn3c(-c4cc(F)c(OCC#N)cc4Cl)cnc23)ccc1C(=O)N1CCC(C(=O)N2CCNC(CO)C2)CC1] | 661.3 | Intermediate 39 and 81 |
| REF 137 | (1-(4-((3-(4-(di- fluoromethoxy)phe- nyl)imidazo[1,2- a]pyrazin-8- yl)amino)-2- methylbenzoyl)pi- peridin-4- yl)((3R,4R,5R)-3,4- dihydroxy-5- (hydroxymethyl)pi- peridin-1- yl)methanone | 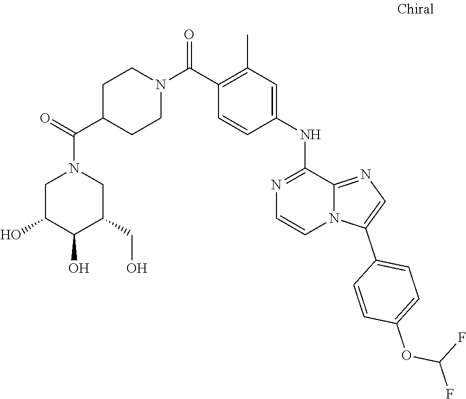 [SMILES: Cc1cc(Nc2nccn3c(-c4ccc(OC(F)F)cc4)cnc23)ccc1C(=O)N1CCC(C(=O)N2C[C@H](CO)[C@@H](O)[C@H](O)C2)CC1] | 651.5 | Intermediate 7 and (3R,4R,5R)-5- (hydroxymethyl)pi- peridine-3,4-diol hydrochloride |
| REF 138 | (4-(amino- methyl)piperi- din-1-yl)(4-((3-(3- fluoro-4- methoxyphenyl)imi- dazo[1,2-a]pyrazin- 8-yl)amino)-2- methyl- phenyl)metha- none hydrochloride | 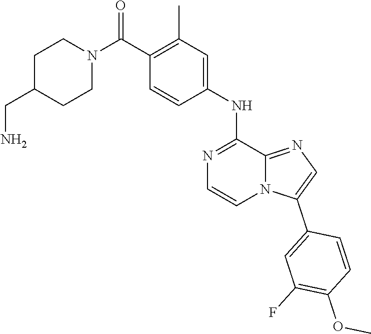 [SMILES: COc1ccc(-c2cnc3c(Nc4ccc(C(=O)N5CCC(CN)CC5)c(C)c4)nccn23)cc1F] | 489.2 | Intermediate 6 and tert-butyl (piperidin-4- ylmethyl)carbamate |
| REF 139 | (4-(2-(dimethyl- amino)ethyl) piperazin-1-yl)(4- ((3-(3-fluoro-4- methoxyphenyl)imi- dazo[1,2-a]pyrazin- 8-yl)amino)-2- methylphenyl)meth- anone | 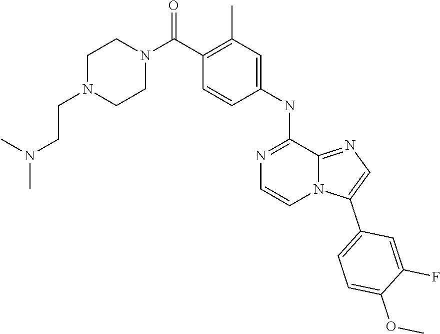 | 532.6 | Intermediate 6 and N,N-dimethyl- 2-(piperazin-1- yl)ethanamine |
| REF 140 | (4-((3-(4-(di- fluoromethoxy)phe- nyl)imidazo[1,2- a]pyrazin-8- yl)amino)-2- methylphenyl)(4-(4- methylpiperazin-1- yl)piperidin-1- yl)methanone | 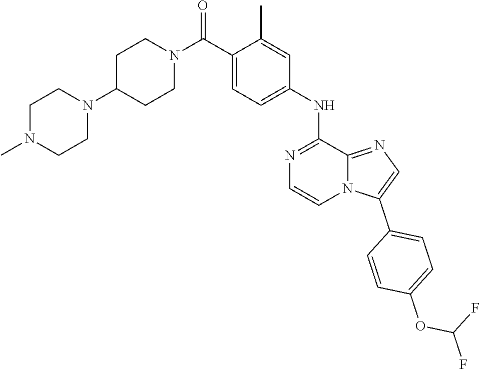 [SMILES: Cc1cc(Nc2nccn3c(-c4ccc(OC(F)F)cc4)cnc23)ccc1C(=O)N1CCC(N2CCN(C)CC2)CC1] | 576.2 | Intermediate 7 and 1-methyl-4- (piperidin-4- yl)piperazine |
| REF 141 | 2-amino-1-(4-(4- ((3-(4-(di- fluoromethoxy)phe- nyl)imidazo[1,2- a]pyrazin-8- yl)amino)-2- methyl- benzoyl)piper- azin-1-yl)ethanone hydrochloride | 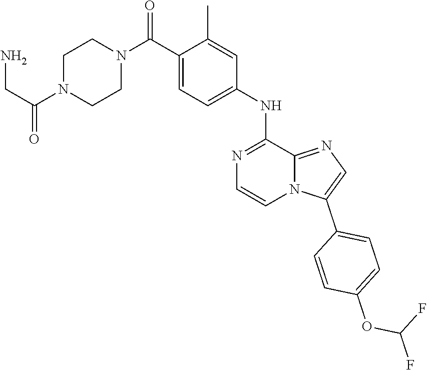 [SMILES: Cc1cc(Nc2nccn3c(-c4ccc(OC(F)F)cc4)cnc23)ccc1C(=O)N1CCN(C(=O)CN)CC1] | 536.2 | Intermediate 7 and tert-butyl (2-oxo-2- (piperazin-1- yl)ethyl)carbamate |
| REF 142 | 1-(4-(4-((3-(4-(di- fluoromethoxy)phe- nyl)imidazo[1,2- a]pyrazin-8- yl)amino)-2- methylbenzoyl)pi- perazin-1-yl)-2- (dimethylamino) ethanone | 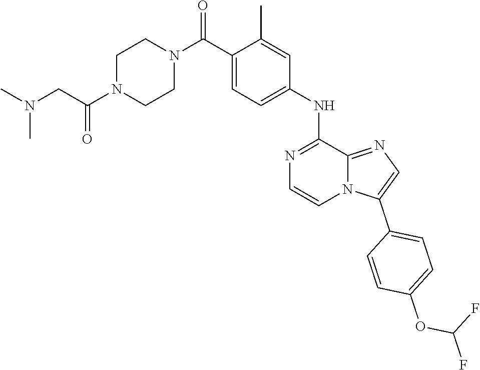 [SMILES: Cc1cc(Nc2nccn3c(-c4ccc(OC(F)F)cc4)cnc23)ccc1C(=O)N1CCN(C(=O)CN(C)C)CC1] | 564.1 | Intermediate 7 and 2- (dimethylamino)-1- (piperazin-1- yl)ethanone dihydrochloride |
| REF 143 | (4-(2-(amino- methyl)morpho- line-4- carbonyl)piperidin- 1-yl)(4-((3-(3- fluoro-4- methoxyphenyl)imi- dazo[1,2-a]pyrazin- 8-yl)amino)-2- methylphe- nyl)metha- none hydrochloride | 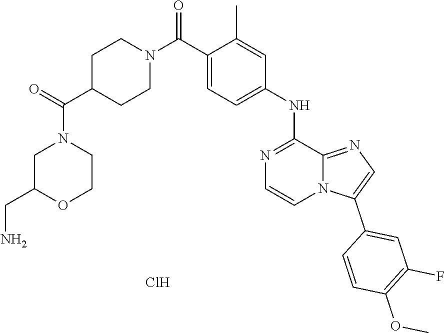 [SMILES: COc1ccc(-c2cnc3c(Nc4ccc(C(=O)N5CCC(C(=O)N6CCOC(CN)C6)CC5)c(C)c4)nccn23)cc1F ; Cl] | 602.3 | Intermediate 6 and tert-butyl (morpholin-2- ylmethyl)carbamate |
| REF 144 | 1-(4-((3-(3-fluoro- 4-methoxy- phenyl)imi- dazo[1,2-a]pyrazin- 8-yl)amino)-2- methylbenzoyl)-N- (2-(methyl- amino)ethyl) piperidine-4- carboxamide hydrochloride | 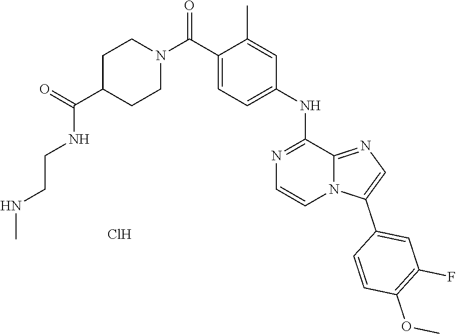 [SMILES: CNCCNC(=O)C1CCN(C(=O)c2ccc(Nc3nccn4c(-c5ccc(OC)c(F)c5)cnc34)cc2C)CC1 ; Cl] | 560.3 | Intermediate 6 and tert-butyl (2- aminoethyl)(methyl) carbamate |
| REF 145 | N-(2-((2-amino- ethyl)(methyl) amino)ethyl)-4-((3- (3-fluoro-4- methoxyphenyl)imi- dazo[1,2-a]pyrazin- 8-yl)amino)-2- methylbenzamide | 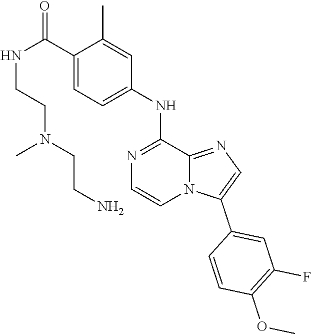 [SMILES: COc1ccc(-c2cnc3c(Nc4ccc(C(=O)NCCN(C)CCN)c(C)c4)nccn23)cc1F] | 492.3 | Intermediate 6 and N1-(2- aminoethyl)-N1- methylethane-1,2- diamine |
| REF 146 | (4-((3-(3-fluoro-4- methoxyphenyl)imi- dazo[1,2-a]pyrazin- 8-yl)amino)-2- methylphenyl)(4-(1- methyl-1H- imidazol- 2-yl)piperazin-1- yl)methanone | 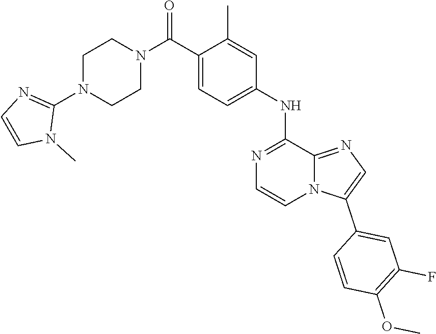 [SMILES: COc1ccc(-c2cnc3c(Nc4ccc(C(=O)N5CCN(c6nccn6C)CC5)c(C)c4)nccn23)cc1F] | 541.3 | Intermediate 6 and 1-(1-methyl- 1H-imidazol-2- yl)piperazine |
| 16 | 1-(2-chloro-4-((3-(3- fluoro-4- methoxyphenyl)imi- dazo[1,2-a]pyrazin- 8-yl)amino)ben- zoyl)-N-(2- (methylamino)ethyl) piperidine-4- carboxamide hydrochloride | 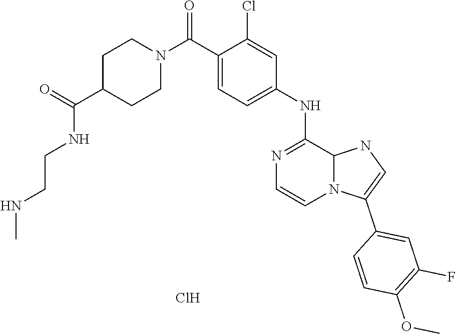 | 580.2 | Intermediate 2 and tert-butyl (2- aminoethyl)(methyl) carbamate |
| REF 147 | (4-((3-(3-fluoro-4- methoxyphenyl)imi- dazo[1,2-a]pyrazin- 8-yl)amino)-2- methylphenyl)(2- (hydroxymethyl)pi- perazin-1- yl)methanone | 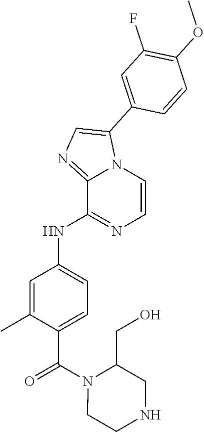 [SMILES: COc1ccc(-c2cnc3c(Nc4ccc(C(=O)N5CCNCC5CO)c(C)c4)nccn23)cc1F] | 489.4 | Intermediate 6 and tert-butyl 2- (hydroxymethyl)piper- azine-1-carboxylate |
| REF 148 | (4-(2- (aminomethyl)- morpholine-4- carbonyl)piperidin- 1-yl)(4-((3-(4-(di- fluoro- methoxy)phe- nyl)imidazo[1,2- a]pyrazin-8- yl)amino)-2- methylphe- nyl)metha- none hydrochloride | 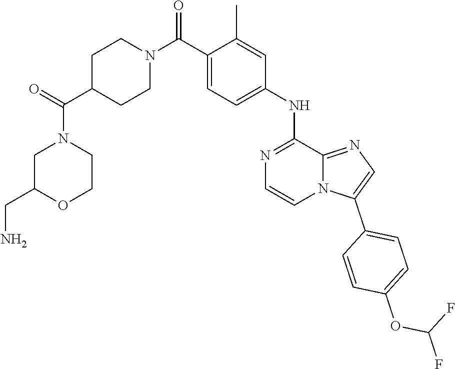 [SMILES: Cc1cc(Nc2nccn3c(-c4ccc(OC(F)F)cc4)cnc23)ccc1C(=O)N1CCC(C(=O)N2CCOC(CN)C2)CC1] | 678.7 | Reference Example 1 and tert-butyl (2- aminoethyl)(methyl) carbamate |
| REF 149 | (4-(6-cyclopropyl- 2,6-diaza- spiro[3.3]heptane- 2- carbonyl)piperidin- 1-yl)(4-((3-(4- (difluoro- methoxy)phe- nyl)imidazo[1,2- a]pyrazin-8- yl)amino)-2- methylphenyl)- methanone | 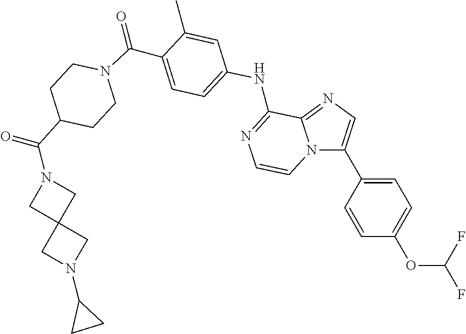 [SMILES: Cc1cc(Nc2nccn3c(-c4ccc(OC(F)F)cc4)cnc23)ccc1C(=O)N1CCC(C(=O)N2CC3(C2)CN(C2CC2)C3)CC1] | 642.5 | Reference Example 1 and 2-cyclopropyl- 2,6- diazaspiro[3.3]heptane |
| REF 150 | (4-((3-(4-(di- fluoromethoxy)phe- nyl)imidazo[1,2- a]pyrazin-8- yl)amino)-2- methylphenyl)(4- (piperazin-1- yl)piperidin-1- yl)methanone hydrochloride | 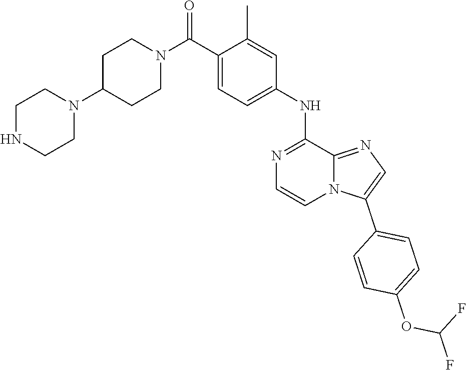 [SMILES: Cc1cc(Nc2nccn3c(-c4ccc(OC(F)F)cc4)cnc23)ccc1C(=O)N1CCC(N2CCNCC2)CC1] | 562.3 | Intermediate 7 and tert-butyl 4- (piperidin-4- yl)piperazine-1- carboxylate |
| REF 151 | N-[3- (dimethylamino)pro- pyl]-1-[4-[[3-(3- fluoro-4- methoxyphenyl)imi- dazo[1,2-a]pyrazin- 8-yl]amino]-2- methylbenzoyl]pi- peridine-4- carboxamide | 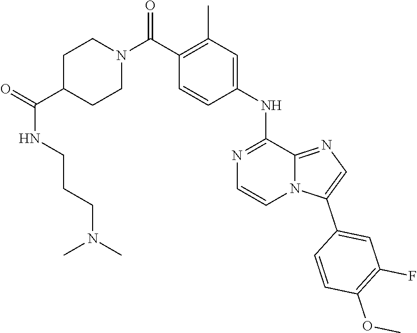 [SMILES: COc1ccc(-c2cnc3c(Nc4ccc(C(=O)N5CCC(C(=O)NCCCN(C)C)CC5)c(C)c4)nccn23)cc1F] | 588.3 | Reference Example 2 and N1,N1- dimethylpropane-1,3- diamine |
| REF 152 | N-[2-(di- methylamino)ethyl]- 1-[4-[[3-(3-fluoro- 4- methoxyphenyl)imi- dazo[1,2-a]pyrazin- 8-yl]amino]-2- methylbenzoyl]pi- peridine-4- carboxamide | 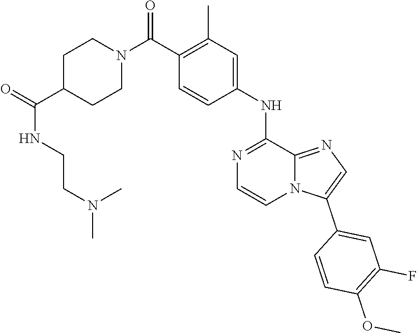 [SMILES: COc1ccc(-c2cnc3c(Nc4ccc(C(=O)N5CCC(C(=O)NCCN(C)C)CC5)c(C)c4)nccn23)cc1F] | 574.7 | Reference Example 2 and N1,N1- dimethylethane-1,2- diamine |
| REF 153 | 4-[1-[4-[[3-(3- fluoro-4-meth- oxyphenyl)imida- zo[1,2-a]pyrazin-8- yl]amino]-2- methylbenzoyl]pi- peridine-4- carbonyl]piperazine- 2-carboxylic acid; hydrochloride | 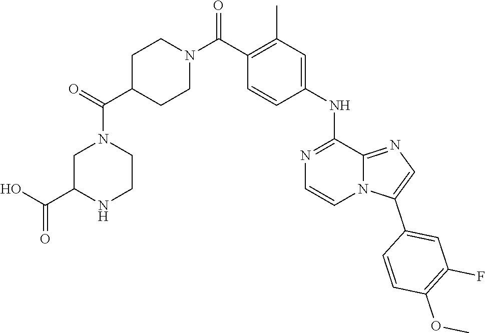 [SMILES: COc1ccc(-c2cnc3c(Nc4ccc(C(=O)N5CCC(C(=O)N6CCNC(C(=O)O)C6)CC5)c(C)c4)nccn23)cc1F] | 616.3 | Reference Example 2 and 1-tert-butyl 2- methyl piperazine- 1,2-dicarboxylate, the intermediate ester was hydrolyzed with LiOH in THF/MeOH/H2O as described previously |
| REF 154 | 1-(4-((3-(3-fluoro-4- methoxyphenyl)imi- dazo[1,2-a]pyrazin- 8-yl)amino)-2- methylbenzoyl)-N- (3- (methylamino)pro- pyl)piperidine-4- carboxamide hydrochloride | 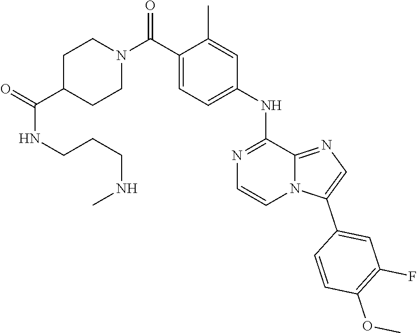 [SMILES: CNCCCNC(=O)C1CCN(C(=O)c2ccc(Nc3nccn4c(-c5ccc(OC)c(F)c5)cnc34)cc2C)CC1] | 574.4 | Reference Example 2 and tert-butyl (3- aminopropyl)(methyl) carbamate |
| REF 155 | (R)-(1-(4-((3-(3- fluoro-4-methoxy- phenyl)imi- dazo[1,2-a]pyrazin- 8-yl)amino)-2- methylbenzoyl)pi- peridin-4-yl)(3- (methylamino)pyr- rolidin-1-yl) methanone hydrochloride | 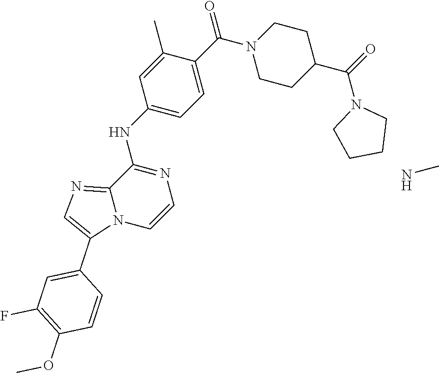 | 586.4 | Reference Example 2 and (R)-tert-butyl methyl(pyrrolidin-3- yl)carbamate |
| REF 156 | 1-(4-((3-(3-fluoro- 4-methoxy- phenyl)imidazo[1,2- a]pyrazin- 8-yl)amino)-2- methylbenzoyl)-N- (4- (methylamino)butyl) piperidine-4- carboxamide hydrochloride | 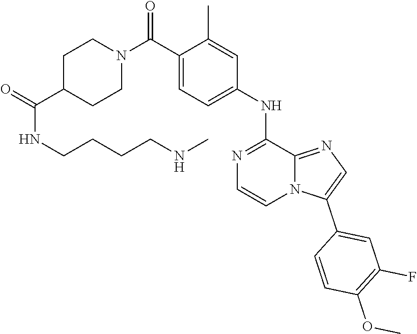 [SMILES: CNCCCCNC(=O)C1CCN(C(=O)c2ccc(Nc3nccn4c(-c5ccc(OC)c(F)c5)cnc34)cc2C)CC1] | 588.5 | Reference Example 2 and tert-butyl (4- aminobutyl)(methyl) carbamate |
| REF 157 | N-(3-aminopropyl)- 1-(4-((3-(3- fluoro-4-methoxy- phenyl)imi- dazo[1,2-a]pyrazin- 8-yl)amino)-2- methylbenzoyl)pi- peridine-4- carboxamide hydrochloride | 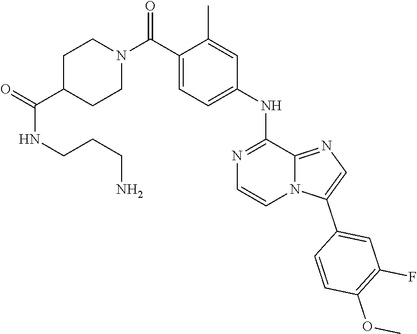 [SMILES: COc1ccc(-c2cnc3c(Nc4ccc(C(=O)N5CCC(C(=O)NCCCN)CC5)c(C)c4)nccn23)cc1F] | 560.4 | Reference Example 2 and tert-butyl (3- aminopropyl)carba- mate |
| REF 158 | (1-(4-((3-(3-fluoro- 4- methoxyphenyl)imida- zo[1,2-a]pyrazin-8- yl)amino)-2- methylbenzoyl)piper- idin-4-yl)(3- (hydroxymethyl)piper- azin-1- yl)methanone 2,2,2- trifluoroacetate | 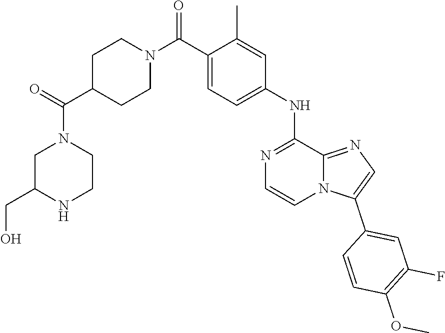 [SMILES: COc1ccc(-c2cnc3c(Nc4ccc(C(=O)N5CCC(C(=O)N6CCNC(CO)C6)CC5)c(C)c4)nccn23)cc1F] | 602.4 | Reference Example 2 and tert-butyl 2- (hydroxymethyl)piper- azine-1-carboxylate |
| REF 159 | 1-(4-((3-(3-fluoro- 4-methoxy- phenyl)imidazo [1,2-a]pyrazin- 8-yl)amino)-2- methylbenzoyl)-N- ((3-hydroxy- pyrrolidin-3- yl)methyl)piperi- dine-4- carboxamide 2,2,2- trifluoroacetate | 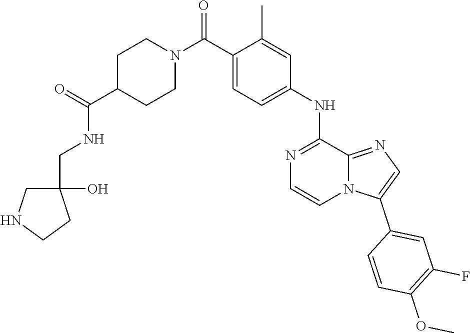 [SMILES: COc1ccc(-c2cnc3c(Nc4ccc(C(=O)N5CCC(C(=O)NCC6(O)CCNC6)CC5)c(C)c4)nccn23)cc1F] | 602.4 | Reference Example 2 and tert-butyl 3- (aminomethyl)-3- hydroxypyrrolidine- 1-carboxylate |
| REF 160 | N-(3-amino-2- hydroxypropyl)-1- (4-((3-(3-fluoro-4- methoxyphenyl)imi- dazo[1,2-a]pyrazin- 8-yl)amino)-2- methyl- benzoyl)piperidine- 4-carboxamide 2,2,2- trifluoroacetate | 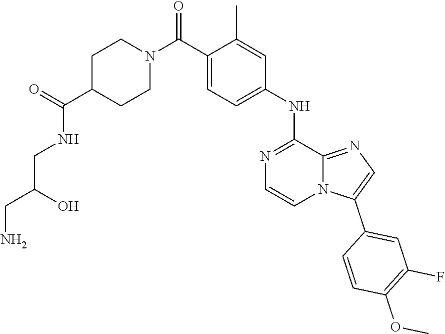 [SMILES: COc1ccc(-c2cnc3c(Nc4ccc(C(=O)N5CCC(C(=O)NCC(O)CN)CC5)c(C)c4)nccn23)cc1F] | 576.3 | Reference Example 2 and tert-butyl (3- amino-2- hydroxypropyl)carba- mate |
| REF 161 | 1-(4-((3-(3-fluoro-4- methoxyphenyl)imi- dazo[1,2-a]pyrazin- 8-yl)amino)-2- methylbenzoyl)-N- ((3R,4R)-4- hydroxypyrrolidin- 3-yl)piperidine-4- carboxamide 2,2,2- trifluoroacetate | 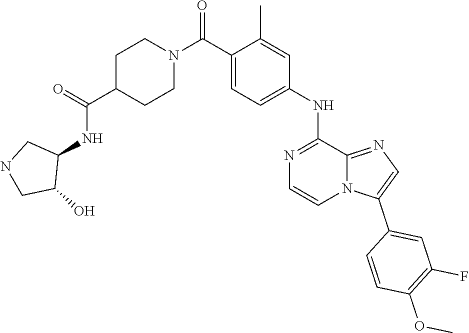 | 588.3 | Reference Example 2 and (3R,4R)-tert- butyl 3-amino-4- hydroxypyrrolidine- 1-carboxylate |
| REF 162 | N-(azetidin-3-yl)-1- (4-((3-(3-fluoro-4- methoxyphenyl)imi- dazo[1,2-a]pyrazin- 8-yl)amino)-2- methylbenzoyl)pi- peridine-4- carboxamide 2,2,2- trifluoroacetate | 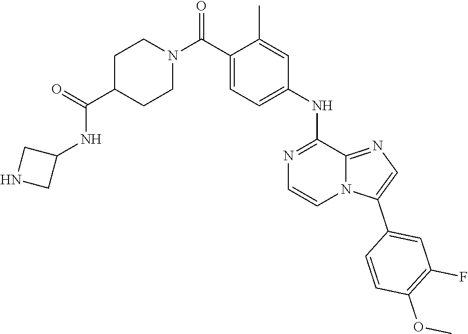 [SMILES: COc1ccc(-c2cnc3c(Nc4ccc(C(=O)N5CCC(C(=O)NC6CNC6)CC5)c(C)c4)nccn23)cc1F] | 558.3 | Reference Example 2 and tert-butyl 3- aminoazetidine-1- carboxylate |
| REF 163 | 1-(4-((3-(3-fluoro-4- methoxyphenyl)imi- dazo[1,2-a]pyrazin- 8-yl)amino)-2- methylbenzoyl)-N- ((3-hydroxy- azetidin- 3-yl)methyl) piperidine- 4-carboxamide 2,2,2- trifluoroacetate | 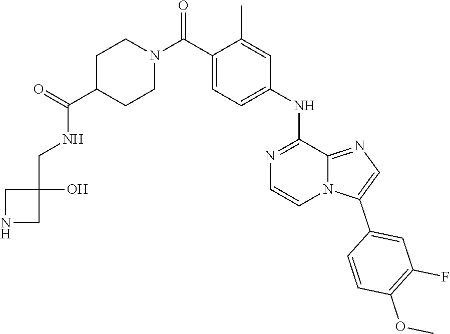 [SMILES: COc1ccc(-c2cnc3c(Nc4ccc(C(=O)N5CCC(C(=O)NCC6(O)CNC6)CC5)c(C)c4)nccn23)cc1F] | 588.3 | Reference Example 2 and tert-butyl 3- (aminomethyl)-3- hydroxyazetidine-1- carboxylate |
| REF 164 | (R)-1-(4-((3-(3- fluoro-4-meth- oxyphenyl)imida- zo[1,2-a]pyrazin-8- yl)amino)-2- methylbenzoyl)-N- (pyrrolidin-3- yl)piperidine-4- carboxamide 2,2,2- trifluoroacetate | 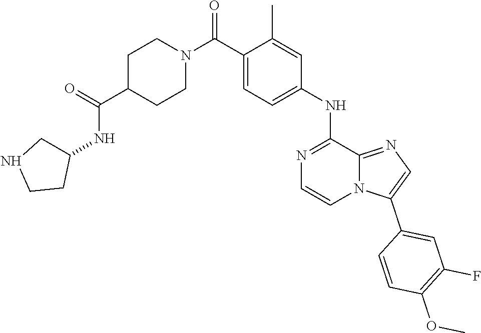 | 572.3 | Reference Example and (R)-tert-butyl 3- aminopyrrolidine-1- carboxylate |
| REF 165 | N-(azetidin-3- ylmethyl)-1-(4-((3- (3-fluoro-4- methoxyphenyl)imi- dazo[1,2-a]pyrazin- 8-yl)amino)-2- methyl- benzoyl)piperi- dine-4-carboxamide 2,2,2- trifluoroacetate | 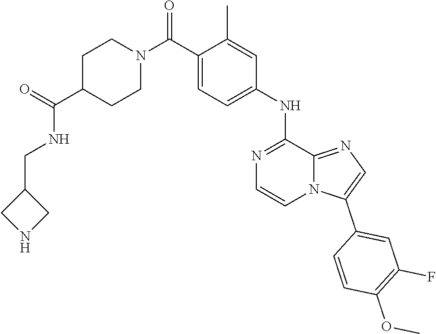 [SMILES: COc1ccc(-c2cnc3c(Nc4ccc(C(=O)N5CCC(C(=O)NCC6CNC6)CC5)c(C)c4)nccn23)cc1F] | 572.4 | Reference Example 2 and tert-butyl 3- (aminomethyl)azeti- dine-1-carboxylate |
| REF 166 | N-(2-(azetidin-1- yl)ethyl)-1-(4- ((3-(3-fluoro-4- methoxyphenyl)imi- dazo[1,2-a]pyrazin- 8-yl)amino)-2- methylbenzoyl)pi- peridine-4- carboxamide | 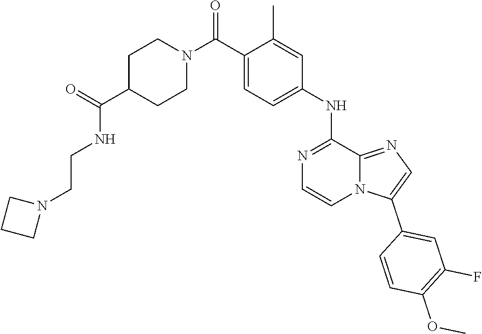 [SMILES: COc1ccc(-c2cnc3c(Nc4ccc(C(=O)N5CCC(C(=O)NCCN6CCC6)CC5)c(C)c4)nccn23)cc1F] | 586.3 | Reference Example 2 and 2-(azetidin-1- yl)ethanamine |
| REF 167 | N-(3-(azetidin-1- yl)propyl)-1-(4-((3- (3-fluoro-4- methoxyphenyl)imi- dazo[1,2-a]pyrazin- 8-yl)amino)-2- methylbenzoyl)pi- peridine-4- carboxamide | 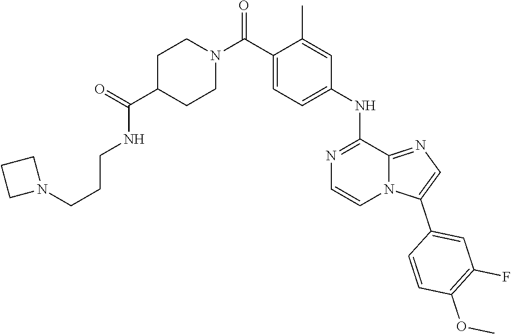 [SMILES: COc1ccc(-c2cnc3c(Nc4ccc(C(=O)N5CCC(C(=O)NCCCN6CCC6)CC5)c(C)c4)nccn23)cc1F] | 600.3 | Reference Example 2 and 3-(azetidin-1- yl)propan-1-amine |
| REF 168 | (R)-1-(4-((3-(2,3- difluoro-4- methoxyphenyl)imi- dazo[1,2-a]pyrazin- 8-yl)amino)-2- methylbenzoyl)-N- (pyrrolidin-3- yl)piperidine-4- carboxamide hydrochloride | 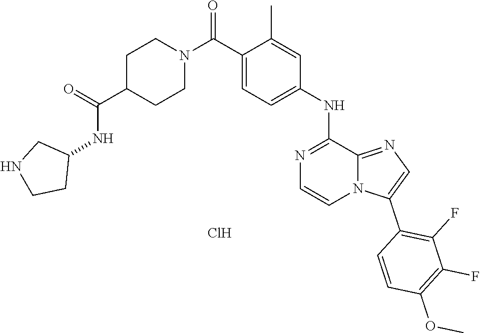 | 590.3 | Intermediate 65 and tert-butyl (R)- 3-aminopyrrolidine- 1-carboxylate |
| 17 | (R)-1-(2-chloro-4- ((3-(3-fluoro-4- methoxyphenyl)imi- dazo[1,2-a]pyrazin- 8-yl)ami- no)benzoyl)- N-(pyrrolidin-3- yl)piperidin-4- carboxamide hydrochlroide | 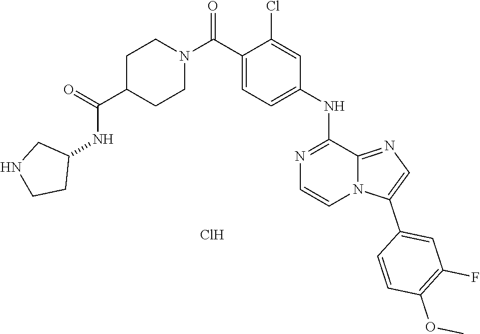 | 593.3 | Intermediate 66 and tert-butyl (R)- 3-aminopyrrolidine- 1-carboxylate |
| 18 | 1-(4-(2-chloro-4- ((3-(3-fluoro-4- methoxyphenyl)imi- dazo[1,2-a]pyrazin- 8-yl)amino) benzoyl)piperazin- 1-yl)-2-(methyl- amino)ethan- 1-one hydrochloride | 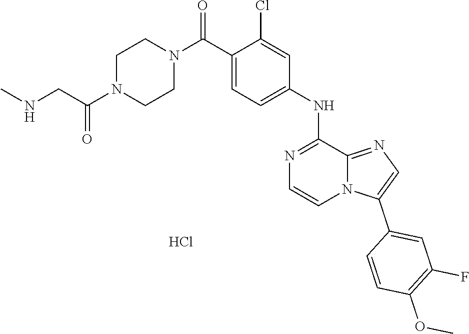 [SMILES: CNCC(=O)N1CCN(C(=O)c2ccc(Nc3nccn4c(-c5ccc(OC)c(F)c5)cnc34)cc2Cl)CC1 ; Cl] | 553.3 | Intermediate 2 and tert-butyl methyl(2- oxo-2-(piperazin-1- yl)ethyl)carbamate |
| REF 169 | 2-(dimethylamino)- 1-(4-(4-((3-(3- fluoro-4- methoxyphenyl)imi- dazo[1,2-a]pyrazin- 8-yl)amino)-2- methylbenzoyl)pi- perazin-1- yl)ethanone | 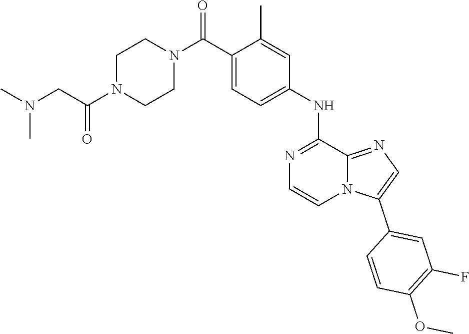 [SMILES: COc1ccc(-c2cnc3c(Nc4ccc(C(=O)N5CCN(C(=O)CN(C)C)CC5)c(C)c4)nccn23)cc1F] | 546.3 | Intermediate 6 and 2- (dimethylamino)-1- (piperazin-1- yl)ethanone dihydrochloride |
| 19 | N-(azetidin-3- ylmethyl)-1-(2- chloro-4-((3-(3- fluoro-4-methoxy- phenyl)imi- dazo[1,2-a]pyrazin- 8-yl)amino)benzoyl) piperidine-4- carboxamide | 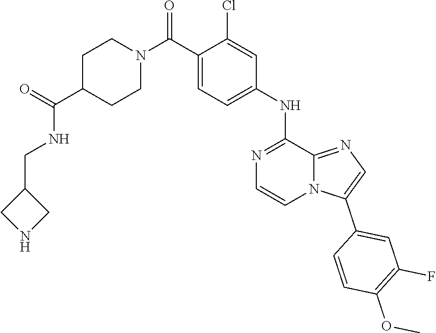 [SMILES: COc1ccc(-c2cnc3c(Nc4ccc(C(=O)N5CCC(C(=O)NCC6CNC6)CC5)c(Cl)c4)nccn23)cc1F] | 590.6 (M − H) | Intermediate 66 and tert-butyl 3- (aminomethyl)azeti- dine-1-carboxylate |
| REF 170 | N-(azetidin-3- ylmethyl)-1-(4-((3- (3-fluoro-4- methoxyphenyl)imi- dazo[1,2-a]pyrazin- 8-yl)amino)-2- methylbenzoyl)pi- peridine- 4-carboxamide | 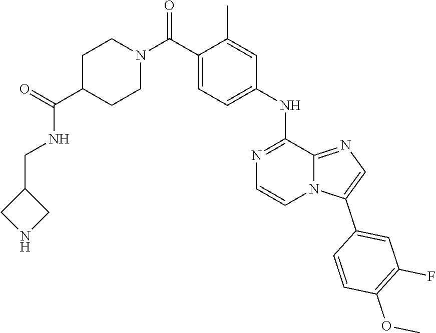 [SMILES: COc1ccc(-c2cnc3c(Nc4ccc(C(=O)N5CCC(C(=O)NCC6CNC6)CC5)c(C)c4)nccn23)cc1F] | 572.5 | Reference Example 2 and tert-butyl 3- (aminomethyl)azeti- dine-1-carboxylate |
| REF 171 | (S)-1-(4-((3-(3- fluoro-4- methoxyphenyl)imi- dazo[1,2-a]pyrazin- 8-yl)amino)-2- methylbenzoyl)-N- (pyrrolidin-3- yl)piperidine-4- carboxamide hydrochloride | 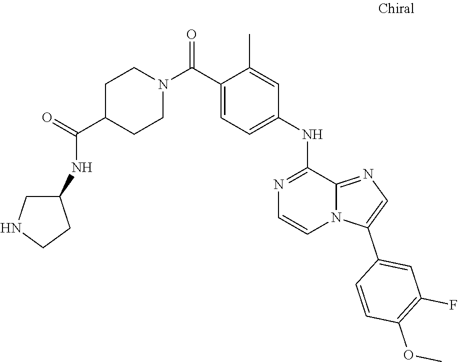 [SMILES: COc1ccc(-c2cnc3c(Nc4ccc(C(=O)N5CCC(C(=O)N[C@H]6CCNC6)CC5)c(C)c4)nccn23)cc1F] | 572.3 | Reference Example 2 and tert-butyl(S)-3- aminopyrrolidine-1- carboxylate |
| REF 172 | (4-((3-(4-(di- fluoromethoxy)phe- nyl)imidazo[1,2- a]pyrazin-8- yl)amino)-2- methylphenyl)(4- (4,5-dihydro-1H- imidazol-2- yl)piperazin-1- yl)methanone | 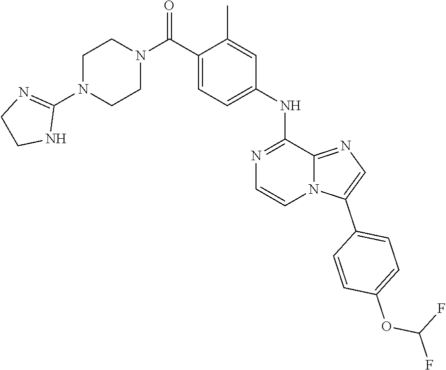 [SMILES: Cc1cc(Nc2nccn3c(-c4ccc(OC(F)F)cc4)cnc23)ccc1C(=O)N1CCN(C2=NCCN2)CC1] | 547.2 | Intermediate 7 and 1- (4,5-dihydro-1H- imidazol-2- yl)piperazine- hydroiodide (CAS 295341-59-2) |
| REF 173 | N-(azetidin-3- ylmethyl)-1-(4-((3- (4-(di- fluoromethoxy)phe- nyl)imidazo[1,2- a]pyrazin-8- yl)amino)-2-meth- ylbenzoyl)piper- idine-4- carboxamide | 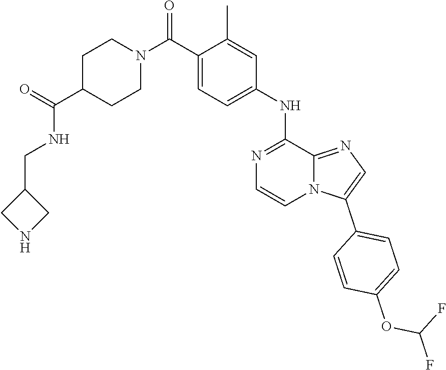 [SMILES: Cc1cc(Nc2nccn3c(-c4ccc(OC(F)F)cc4)cnc23)ccc1C(=O)N1CCC(C(=O)NCC2CNC2)CC1] | 590.4 | Reference Example 1 and tert-butyl 3- (aminomethyl)azeti- dine-1-carboxylate |
| REF 174 | 4-[[3-[4-(di- fluoromethoxy)phe- nyl]imidazo[1,2- a]pyrazin-8- yl]amino]-N,2- dimethyl-N-[2-(4- piperidyl)ethyl] benzamide; hydrochloride | 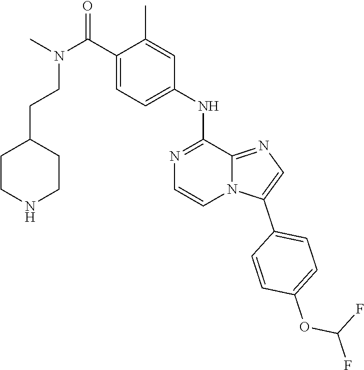 [SMILES: Cc1cc(Nc2nccn3c(-c4ccc(OC(F)F)cc4)cnc23)ccc1C(=O)N(C)CCC1CCNCC1] | 535.5 | Intermediate 7 and tert-butyl 4-(2- (methylamino)eth- yl)piperidine-1- carboxylate |
| REF 175 | N-((1-carba- mimidoylpiperi- din-4-yl)methyl)-4- ((3-(3-chloro-4- methoxyphenyl)imi- dazo[1,2-a]pyrazin- 8-yl)amino)-2- methylbenzamide | 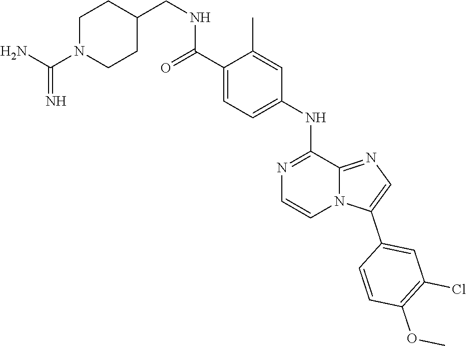 [SMILES: COc1ccc(-c2cnc3c(Nc4ccc(C(=O)NCC5CCN(C(=N)N)CC5)c(C)c4)nccn23)cc1Cl] | 547.4 | Reference Example 2 and 4-(amino- methyl)piperidine- 1- carboximidamide |
| REF 176 | 4-(4-((3-(4-(di- fluoromethoxy)phe- nyl)imidazo[1,2- a]pyrazin-8- yl)amino)-2- methylbenzoyl)pi- perazine-1- carboximidamide | 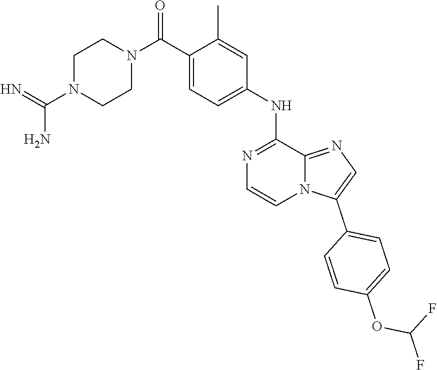 [SMILES: Cc1cc(Nc2nccn3c(-c4ccc(OC(F)F)cc4)cnc23)ccc1C(=O)N1CCN(C(=N)N)CC1] | 521.2 | Intermediate 7 and piperazine-1- carboximidamide |
| REF 177 | 4-((3-(4-(di- fluoromethoxy)phe- nyl)imidazo[1,2- a]pyrazin-8- yl)amino)-N-(4- guanidinobutyl)-2- methylbenzamide | 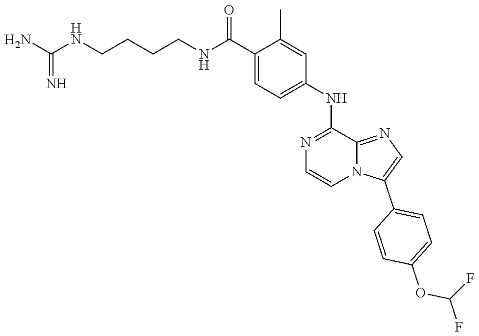 [SMILES: Cc1cc(Nc2nccn3c(-c4ccc(OC(F)F)cc4)cnc23)ccc1C(=O)NCCCCNC(=N)N] | 523.2 | Intermediate 7 and 1- (4- aminobutyl)guanidine sulfate |
| REF 178 | 1-(4-((3-(3-fluoro-4- methoxyphenyl)imi- dazo[1,2-a]pyrazin- 8-yl)amino)-2- methylbenzoyl)-N- ((1-methylazetidin- 3-yl)meth- yl)piperidine- 4-carboxamide | 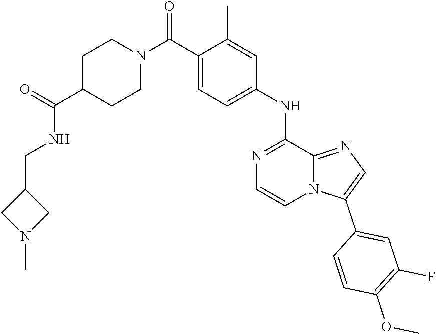 [SMILES: COc1ccc(-c2cnc3c(Nc4ccc(C(=O)N5CCC(C(=O)NCC6CN(C)C6)CC5)c(C)c4)nccn23)cc1F] | 586.3 | Reference Example 2 and (1- methylazetidin-3- yl)methanamine |
| 20 | (2-chloro-4-((3-(3- fluoro-4-methoxy- phenyl)imida- zo[1,2-a]pyrazin-8- yl)amino)phenyl)(4- (2- (methylamino)ethyl) piperazin-1- yl)methanone | 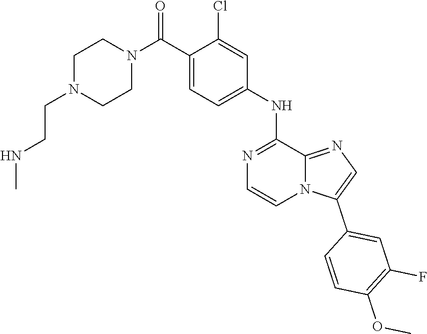 [SMILES: CNCCN1CCN(C(=O)c2ccc(Nc3nccn4c(-c5ccc(OC)c(F)c5)cnc34)cc2Cl)CC1] | 538.2 | Intermediate 2 and (9H-fluoren-9- yl)methyl methyl(2- (piperazin-1- yl)ethyl)carbamate hydrochloride. In situ deprotection with piperidine. |
| REF 125 | 2-chloro-4-[[3-[4- (cyanomethoxy)- 2,3-difluoro- phenyl]imidazo[1,2- a]pyrazin-8- yl]amino]-N-[3-(4- pyridyl)propyl] benzamide; 2,2,2- trifluoroacetic acid | 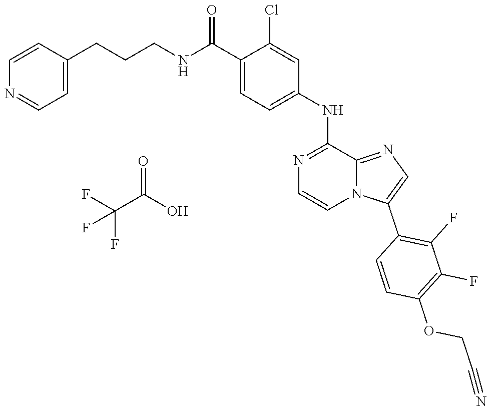 [SMILES: N#CCOc1ccc(-c2cnc3c(Nc4ccc(C(=O)NCCCc5ccncc5)c(Cl)c4)nccn23)c(F)c1F ; O=C(O)C(F)(F)F] | 574.1 | Intermediate 29 and 3-(pyridin-4- yl)propan-1-amine |
| 21 | 2-[4-[8-[3-chloro-4- [4-(4-pyridyl) piperidine-1-car- bonyl]anilino]imi- dazo[1,2-a]pyrazin- 3-yl]-2,3-difluoro- phenoxy]acetonitrile | 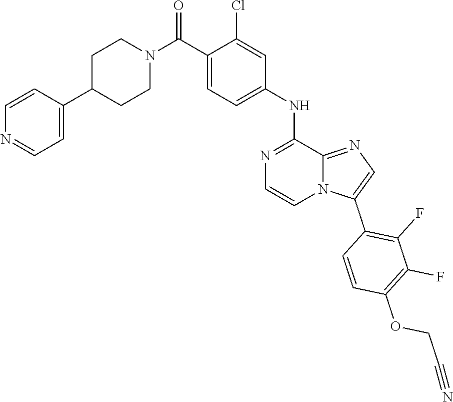 [SMILES: N#CCOc1ccc(-c2cnc3c(Nc4ccc(C(=O)N5CCC(c6ccncc6)CC5)c(Cl)c4)nccn23)c(F)c1F] | 600.1 | Intermediate 29 and 4-(piperidin-4- yl)pyridine |
| REF 179 | 2-chloro-4-[[3-[2- chloro-4- (cyanomethoxy)-3- fluoro- phenyl]imidazo[1,2- a]pyrazin-8- yl]amino]-N-[2-(4- pyridyl)ethyl]benza- mide | 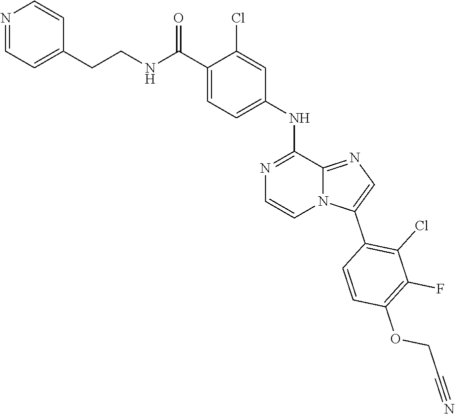 [SMILES: N#CCOc1ccc(-c2cnc3c(Nc4ccc(C(=O)NCCc5ccncc5)c(Cl)c4)nccn23)c(Cl)c1F] | 576.1 | Intermediate 24 and 2-(pyridin-4- yl)ethan-1-amine |
| 22 | 2-[4-[8-[3-chloro-4- (4-pyrimidin-2- ylpiperazine-1-car- bonyl)anilino]imi- dazo[1,2-a]pyrazin- 3-yl]-2,3-difluoro- phenoxy]acetonitrile | 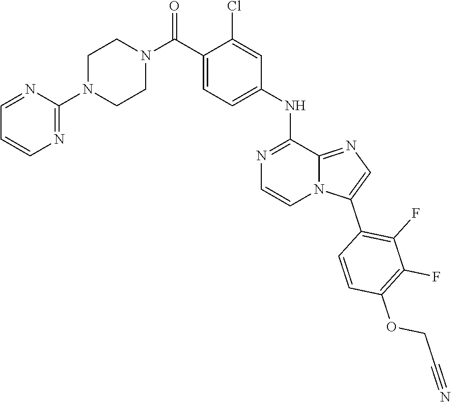 [SMILES: N#CCOc1ccc(-c2cnc3c(Nc4ccc(C(=O)N5CCN(c6ncccn6)CC5)c(Cl)c4)nccn23)c(F)c1F] | 602.1 | Intermediate 29 and 2-(piperazin-1- yl)pyrimidine- hydrochloride |
| 23 | 2-[4-[8-[3-chloro-4- [4-(4-methyl-1,2,4- triazol-3-yl)pi- peridine-1-car- bonyl]anilino]imi- dazo[1,2-a]pyrazin- 3-yl]-2,3-difluoro- phenoxy]aceto- nitrile; 2,2,2- trifluoroacetic acid | 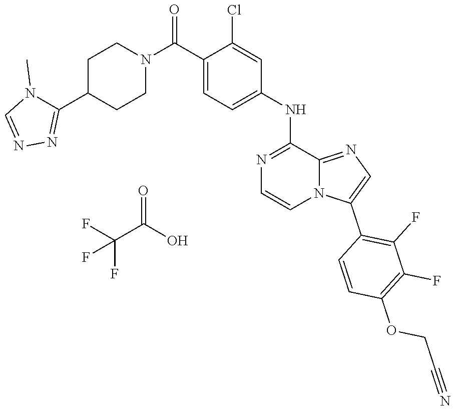 [SMILES: Cn1cnnc1C1CCN(C(=O)c2ccc(Nc3nccn4c(-c5ccc(OCC#N)c(F)c5F)cnc34)cc2Cl)CC1 ; O=C(O)C(F)(F)F] | 604.1 | Intermediate 29 and 4-(4-methyl-4H- 1,2,4-triazol-3- yl)piperidine- hydrochloride |
| REF 180 | 2-chloro-4-[[3-[4- (cyanomethoxy)- 2,3-difluoro- phenyl]imidazo[1,2- a]pyrazin-8- yl]amino]-N-[(2- oxo-1H-pyridin-4- yl)methyl]benz- amide | 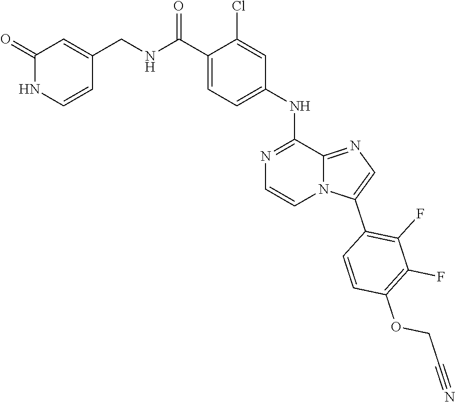 [SMILES: N#CCOc1ccc(-c2cnc3c(Nc4ccc(C(=O)NCc5cc[nH]c(=O)c5)c(Cl)c4)nccn23)c(F)c1F] | 562.1 | Intermediate 29 and 4-(amino- methyl)pyridin- 2(1H)-one hydrochloride |
| REF 181 | 2-chloro-4-[[3-[4- (cyanomethoxy)- 2,3-difluoro- phenyl]imidazo[1,2- a]pyrazin-8- yl]amino]-N-[(1- methyl-2-oxo-4- pyridyl)methyl] benzamide | 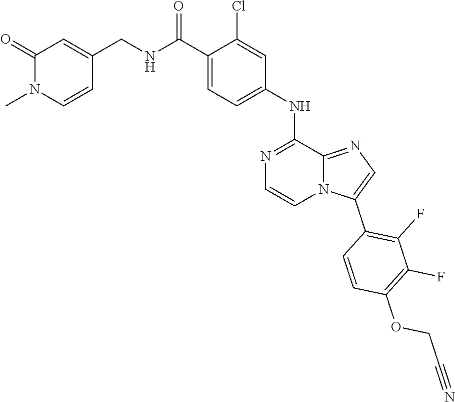 [SMILES: Cn1ccc(CNC(=O)c2ccc(Nc3nccn4c(-c5ccc(OCC#N)c(F)c5F)cnc34)cc2Cl)cc1=O] | 576.1 | Intermediate 29 and 4-(aminomethyl)-1- methylpyridin-2(1H)- one hydrochloride |
| REF 182 | 2-chloro-4-[[3-[4- (cyanomethoxy)- 2,3-difluoro- phenyl]imidazo[1,2- a]pyrazin-8- yl]amino]-N-[(1- methyl-6-oxo-3- pyridyl)methyl] benzamide | 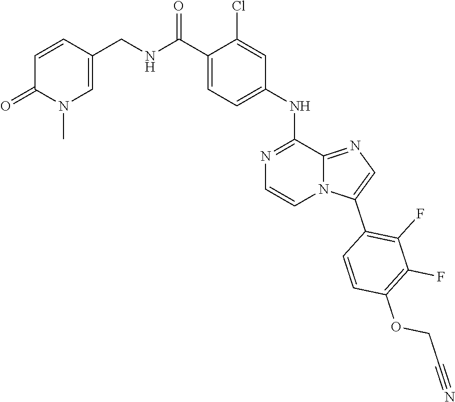 [SMILES: Cn1cc(CNC(=O)c2ccc(Nc3nccn4c(-c5ccc(OCC#N)c(F)c5F)cnc34)cc2Cl)ccc1=O] | 576.1 | Intermediate 29 and 5-(aminomethyl)-1- methylpyridin-2(1H)- one hydrochloride |
| 24 | 2-[4-[8-[3-chloro-4- [4-(1,2,4-triazol-4- yl)piperidine-1- carbonyl]anili- no]imi- dazo[1,2-a]pyrazin- 3-yl]-2,3-difluoro- phenoxy]acetonitrile | 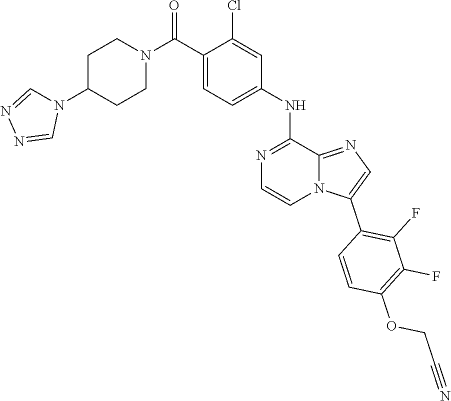 [SMILES: N#CCOc1ccc(-c2cnc3c(Nc4ccc(C(=O)N5CCC(n6cnnc6)CC5)c(Cl)c4)nccn23)c(F)c1F] | 590.1 | Intermediate 29 and 4-(4H-1,2,4-triazol-4- yl)piperidine |
| 11 | 2-[4-[8-[3-chloro-4- [4-(1H-imidazol-4- yl)piperidine-1-car- bonyl]anilino]imi- dazo[1,2-a]pyrazin- 3-yl]-2,3-difluoro- phenoxy]aceto- nitrile; 2,2,2-trifluoroacetic acid | 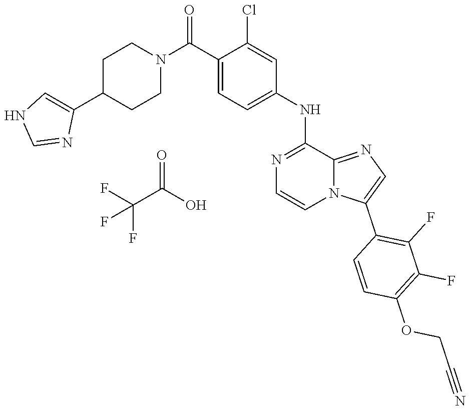 [SMILES: N#CCOc1ccc(-c2cnc3c(Nc4ccc(C(=O)N5CCC(c6c[nH]cn6)CC5)c(Cl)c4)nccn23)c(F)c1F ; O=C(O)C(F)(F)F] | 589.1 | Intermediate 29 and 4-cyclohexyl-1H- imidazole |
| 25 | 2-[3-chloro-4-[8-[3- chloro-4-[4-[2-(di- methylamino)ethyl- amino]piperidine-1- carbonyl]anili- no]imidazo[1,2- a]pyrazin-3-yl]- 2-fluorophenoxy] acetonitrile; 2,2,2-trifluoroacetic acid | 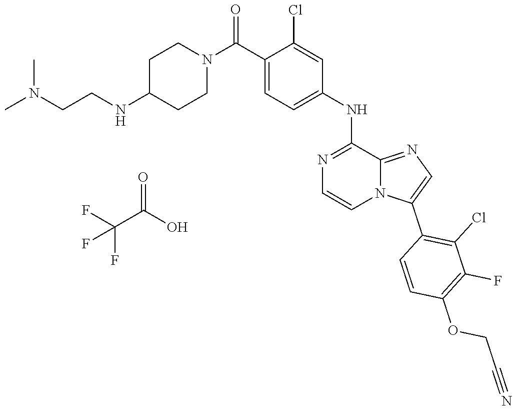 [SMILES: CN(C)CCNC1CCN(C(=O)c2ccc(Nc3nccn4c(-c5ccc(OCC#N)c(F)c5Cl)cnc34)cc2Cl)CC1 ; O=C(O)C(F)(F)F] | 625.1 | Intermediate 24 and N,N-dimethyl-3- (piperazin-1- yl)propan-1-amine |
| 26 | 2-[4-[8-[3-chloro-4- [4-(2-pyrrolidin-1- ylethyl)pipera- zine-1-carbonyl]ani- lino]imi- dazo[1,2-a]pyrazin- 3-yl]-2,3-difluoro- phenoxy]aceto- nitrile; 2,2,2-trifluoroacetic acid | 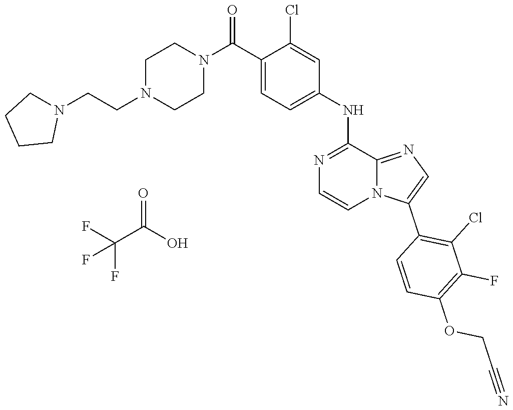 [SMILES: N#CCOc1ccc(-c2cnc3c(Nc4ccc(C(=O)N5CCN(CCN6CCCC6)CC5)c(Cl)c4)nccn23)c(Cl)c1F ; O=C(O)C(F)(F)F] | 637.2 | Intermediate 24 and 1-(2-(pyrrolidin-1- yl)ethyl)piperazine |
| REF 183 | N-(2-(2- aminoethoxy)ethyl)- 4-((3-(4-methoxy- phenyl)imida- zo[1,2-a]pyrazin-8- yl)amino)-N,2- dimethylbenzamide hydrochloride | 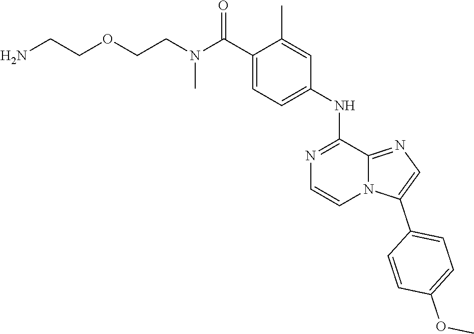 [SMILES: COc1ccc(-c2cnc3c(Nc4ccc(C(=O)N(C)CCOCCN)c(C)c4)nccn23)cc1] | 475.3 | Intermediate 4 and tert-butyl (2-(2- (methylamino)ethoxy) ethyl)carbamate hydrochloride |
| REF 184 | 4-((3-(4-chloro- phenyl)imidazo [1,2-a]pyrazin-8- yl)amino)-N,2- dimethyl-N-(2- (piperidin-4- yl)ethyl)benzamide hydrochloride | 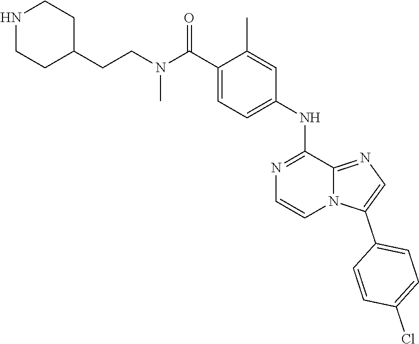 [SMILES: Cc1cc(Nc2nccn3c(-c4ccc(Cl)cc4)cnc23)ccc1C(=O)N(C)CCC1CCNCC1] | 503.4 | Intermediate 82 and tert-butyl 4- (2- (methylamino)ethyl) piperidine-1- carboxylate |
| REF 185 | 4-((3-(4- (difluoromethoxy)- 3-fluoro- phenyl)imidazo [1,2-a]pyrazin-8- yl)amino)-N,2- dimethyl-N-(2- (piperidin-4- yl)ethyl)benzamide hydrochloride | 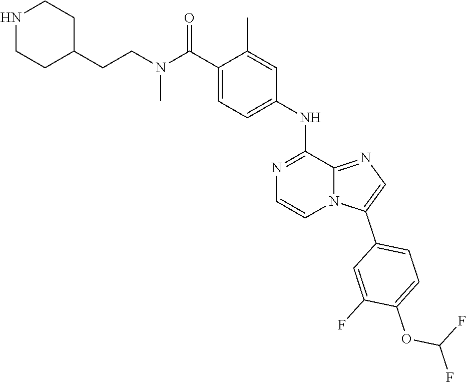 [SMILES: Cc1cc(Nc2nccn3c(-c4ccc(OC(F)F)c(F)c4)cnc23)ccc1C(=O)N(C)CCC1CCNCC1] | 551.5 (M − H) | Intermediate 43 tert-butyl 4-(2- (methylamino)ethyl) piperidine-1- carboxylate |
| REF 186 | 2-chloro-4-((3-(3- fluoro-4-methoxy- phenyl)imida- zo[1,2-a]pyrazin-8- yl)amino)-N- methyl- N-(2-(piperazin-1- yl)ethyl)benzamide hydrochloride | 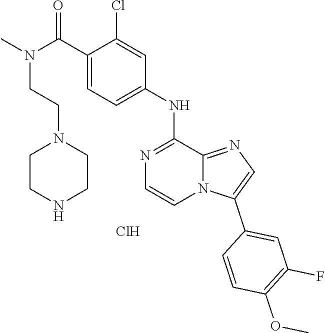 [SMILES: COc1ccc(-c2cnc3c(Nc4ccc(C(=O)N(C)CCN5CCNCC5)c(Cl)c4)nccn23)cc1F ; Cl] | 539.7 | Intermediate 2 and tert-butyl 4-(2- (methylamino)ethyl) piperazine-1- carboxylate |
| 27 | 2-(4-(amino- methyl)piperi- dine-1-carbonyl)-5- ((3-(4-(difluoro- methoxy)phe- nyl)imidazo[1,2- a]pyrazin-8- yl)amino)benzo- nitrile | 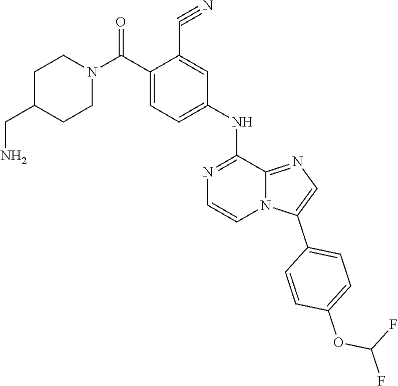 [SMILES: N#Cc1cc(Nc2nccn3c(-c4ccc(OC(F)F)cc4)cnc23)ccc1C(=O)N1CCC(CN)CC1] | 518.2 | Intermediate 45 and tert-butyl (piperidin- 4- ylmethyl)carbamate (Deprotection with TFA) |
| REF 187 | 4-((3-(4-(di- fluoromethoxy)phe- nyl)imidazo[1,2- a]pyrazin-8- yl)amino)-2-iodo-N- methylbenzamide | 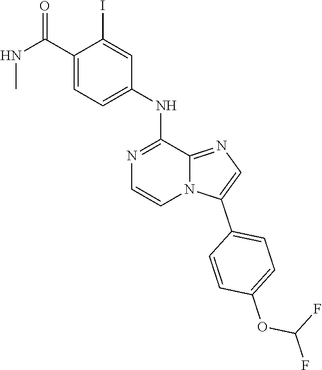 [SMILES: CNC(=O)c1ccc(Nc2nccn3c(-c4ccc(OC(F)F)cc4)cnc23)cc1I] | 536 | Intermediate 46 and methanamine hydrochloride |
| REF 188 | 4-((3-(4-(di- fluoromethoxy)phe- nyl)imidazo[1,2- a]pyrazin-8-yl) amino)-N-methyl- 2-vinylbenzamide | 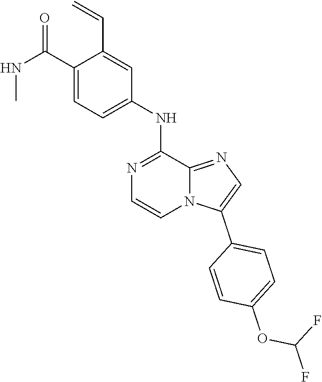 [SMILES: C=Cc1cc(Nc2nccn3c(-c4ccc(OC(F)F)cc4)cnc23)ccc1C(=O)NC] | 436.2 | Intermediate 47 and methanamine hydrochloride |
| 83 | 2-bromo-4-((3- (4-(di- fluoromethoxy)phe- nyl)imidazo[1,2- a]pyrazin-8- yl)amino)-N- methylbenzamide | 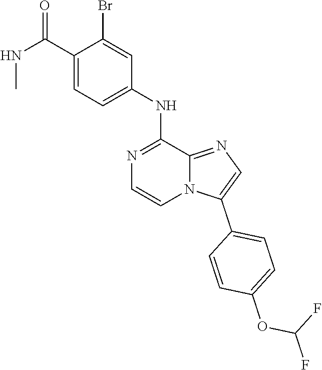 [SMILES: CNC(=O)c1ccc(Nc2nccn3c(-c4ccc(OC(F)F)cc4)cnc23)cc1Br] | 488.1 | Intermediate 10 and methanamine hydrochloride |
| REF 189 | [4-(aminomethyl)-4- hydroxypiperidin-1- yl]-[4-[[3-(3-fluoro- 4-methoxy- phenyl)imida- zo[1,2-a]pyrazin-8- yl]amino]-2- methylphenyl]metha- none; 2,2,2- trifluoroacetic acid | 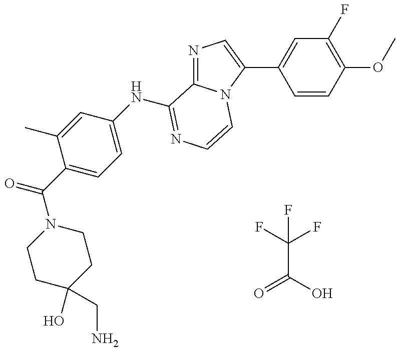 [SMILES: COc1ccc(-c2cnc3c(Nc4ccc(C(=O)N5CCC(O)(CN)CC5)c(C)c4)nccn23)cc1F ; O=C(O)C(F)(F)F] | 505.3 | Intermediate 6 and tert-butyl ((4- hydroxypiperidin-4- yl)methyl)carbamate |
| REF 190 | (4-((1H-imidazol-4- yl)methyl)piperidin- 1-yl)(4-((3-(4-(di- fluoromethoxy)phe- nyl)imidazo[1,2- a]pyrazin-8- yl)amino)-2- methylphe- nyl)methanone | 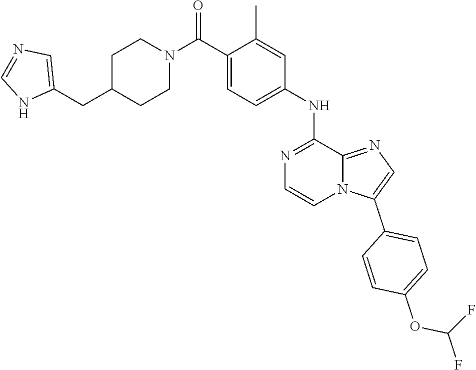 [SMILES: Cc1cc(Nc2nccn3c(-c4ccc(OC(F)F)cc4)cnc23)ccc1C(=O)N1CCC(Cc2cnc[nH]2)CC1] | 558.1 | Intermediate 7 and 4-((1H- imidazol-4- yl)methyl)piperidine- dihydrobromide |
| REF 191 | N-(2-((2-amino- ethyl)thio)ethyl)- 4-((3-(3-fluoro-4- methoxyphenyl)imi- dazo[1,2-a]pyra- zin-8-yl)amino)-2- methylbenzamide | 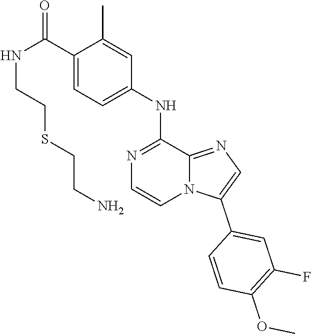 [SMILES: COc1ccc(-c2cnc3c(Nc4ccc(C(=O)NCCSCCN)c(C)c4)nccn23)cc1F] | 495.2 | Intermediate 6 and 2,2′- thiodiethanamine |
| REF 192 | (4-(azetidin-3- yl)piperazin-1-yl)(4- ((3-(3-fluoro-4- methoxy- phenyl)imida- zo[1,2-a]pyrazin-8- yl)amino)-2- methylphenyl)metha- none hydrochloride | 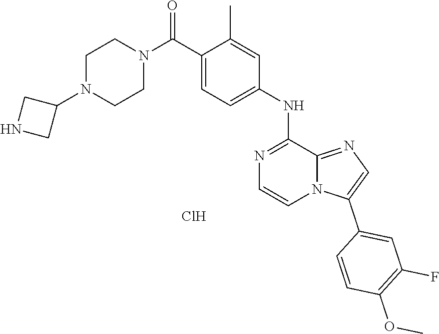 [SMILES: COc1ccc(-c2cnc3c(Nc4ccc(C(=O)N5CCN(C6CNC6)CC5)c(C)c4)nccn23)cc1F ; Cl] | 516.3 | Intermediate 6 and tert-butyl 3- (piperazin-1- yl)azetidine-1- carboxylate |
| 84 | N-(2-((2-amino- ethyl)thio)ethyl)- 4-((3-(4-(di- fluoromethoxy)phe- nyl)imidazo[1,2- a]pyrazin-8- yl)amino)-2- methylbenzamide | 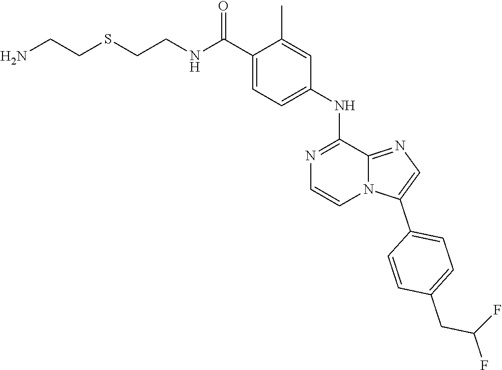 [SMILES: Cc1cc(Nc2nccn3c(-c4ccc(CC(F)F)cc4)cnc23)ccc1C(=O)NCCSCCN] | 513.3 | Intermediate 7 and 2,2′- thiodiethanamine |
| REF 193 | N-(4-aminobutyl)- 4-((3-(4- (difluoromethoxy)- 2,3-difluoro- phenyl)imida- zo[1,2-a]pyrazin-8- yl)amino)-2- ethylbenzamide hydrochloride | 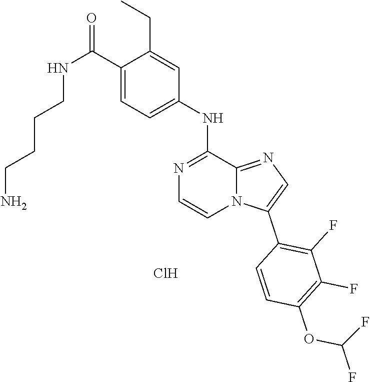 [SMILES: CCc1cc(Nc2nccn3c(-c4ccc(OC(F)F)c(F)c4F)cnc23)ccc1C(=O)NCCCCN ; Cl] | 531.3 | Intermediate 85 and tert-butyl (4- aminobutyl)carba- mate |
| REF 194 | 4-(4-((3-(4-(di- fluoromethoxy)phe- nyl)imidazo[1,2- a]pyrazin-8- yl)amino)-2- methyl- benzoyl)piperazine- 1-carboxamide | 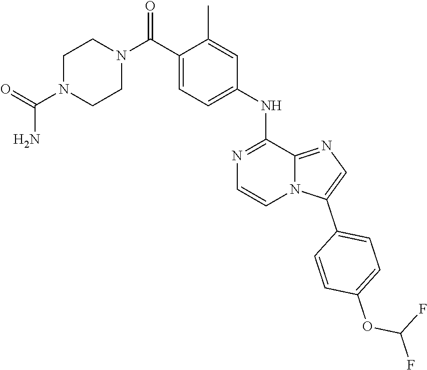 [SMILES: Cc1cc(Nc2nccn3c(-c4ccc(OC(F)F)cc4)cnc23)ccc1C(=O)N1CCN(C(N)=O)CC1] | 522.2 | Intermediate 7 and piperazine-1- carboximidamide |
| REF 195 | N-(azetidin-3- ylmethyl)-1-(4-((3- (2,3-difluoro-4- methoxy- phenyl)imida- zo[1,2-a]pyrazin-8- yl)amino)-2- methyl- benzoyl)piperi- dine-4-carboxamide hydrochloride | 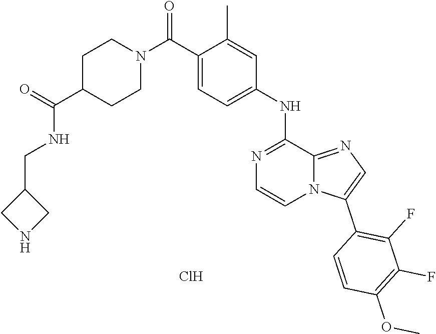 [SMILES: COc1ccc(-c2cnc3c(Nc4ccc(C(=O)N5CCC(C(=O)NCC6CNC6)CC5)c(C)c4)nccn23)c(F)c1F ; Cl] | 590.3 | Intermediate 65 and benzyl 3- (aminomethyl)azeti- dine-1-carboxylate |
| 28 | (2-chloro-4-((3-(3- fluoro-4-methoxy- phenyl)imida- zo[1,2-a]pyrazin-8- yl)amino)phenyl)(4- (2-(dimethyl- amino)ethyl)- 4- hydroxypiperidin-1- yl)methanone | 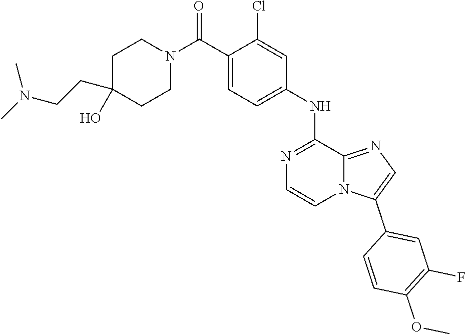 [SMILES: COc1ccc(-c2cnc3c(Nc4ccc(C(=O)N5CCC(O)(CCN(C)C)CC5)c(Cl)c4)nccn23)cc1F] | 567.2 | Intermediate 2 and 4-(2-(di- methylamino)ethyl) piperidin-4-ol |
| REF 196 | 4-((3-(4- (difluoromethoxy)- 2,3-difluoro- phenyl)imida- zo[1,2-a]pyrazin-8- yl)amino)-2-ethyl-N- (3-(pyrrolidin-1- yl)propyl)benzamide | 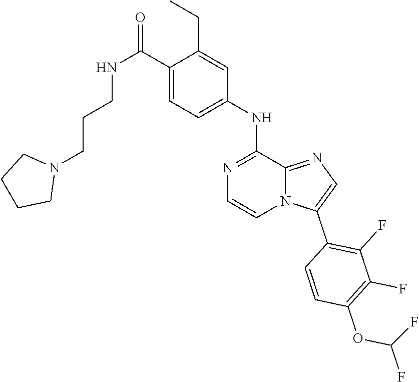 [SMILES: CCc1cc(Nc2nccn3c(-c4ccc(OC(F)F)c(F)c4F)cnc23)ccc1C(=O)NCCCN1CCCC1] | 571.4 | Intermediate 85 and 3-(pyrrolidin-1- yl)propan-1-amine |
| REF 197 | N-(5-aminopentyl)- 4-((3-(4- (difluoromethoxy)- 2,3-difluoro- phenyl)imida- zo[1,2-a]pyrazin-8- yl)amino)-2- ethylbenzamide hydrochlroide yl)amino)-2- ethylbenzamide hydrochloride | 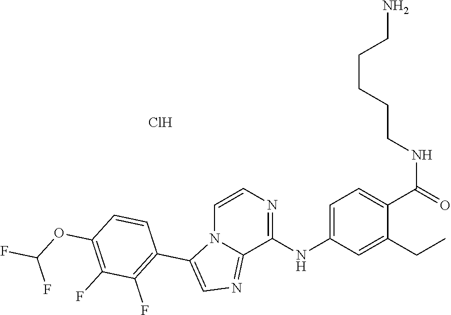 [SMILES: CCc1cc(Nc2nccn3c(-c4ccc(OC(F)F)c(F)c4F)cnc23)ccc1C(=O)NCCCCCN ; Cl] | 545.3 | Intermediate 85 and tert-butyl (5- aminopentyl)carba- mate |
| REF 198 | (R)-4-((3-(2,3- difluoro-4- methoxy- phenyl)imida- zo[1,2-a]pyrazin-8- yl)amino)-2-ethyl- N-(pyrrolidin-3- yl)benzamide hydrochloride | 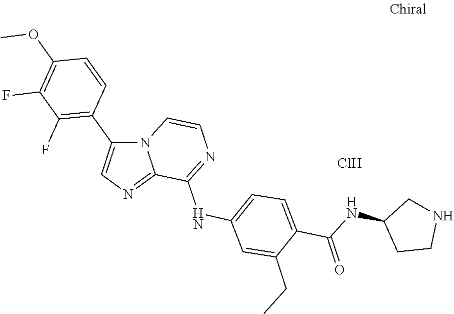 [SMILES: CCc1cc(Nc2nccn3c(-c4ccc(OC)c(F)c4F)cnc23)ccc1C(=O)N[C@@H]1CCNC1 ; Cl] | | Intermediate 86 and rac-tert-butyl (R)-3- aminopyrrolidine-1- carboxylate |
| REF 199 | (S)-N-(4- aminobutan-2-yl)-4- ((3-(2,3-difluoro-4- methoxy- phenyl)imida- zo[1,2-a]pyrazin-8- yl)amino)-2- ethylbenzamide hydrochloride | 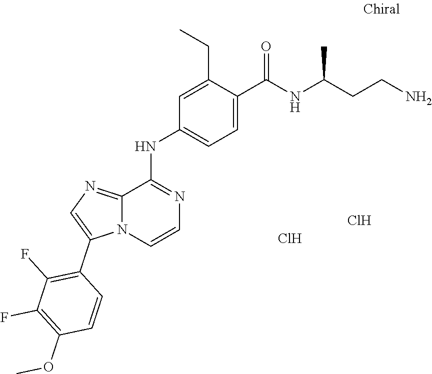 [SMILES: CCc1cc(Nc2nccn3c(-c4ccc(OC)c(F)c4F)cnc23)ccc1C(=O)N[C@@H](C)CCN ; Cl ; Cl] | | Intermediate 86 and rac-tert-butyl (R)-(3-amino- butyl)carbamate |
| REF 200 | N-(3-amino-2,2- dimethylpropyl)-4- ((3-(2,3-difluoro-4- methoxy- phenyl)imida- zo[1,2-a]pyrazin-8- yl)amino)-2- ehtylbenzamide hydrochloride | 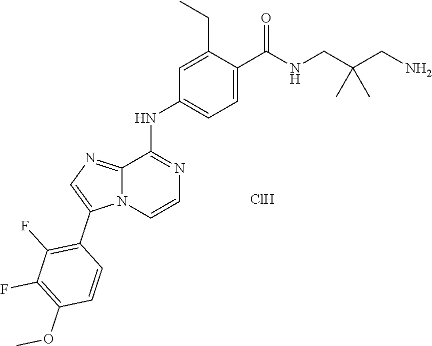 [SMILES: CCc1cc(Nc2nccn3c(-c4ccc(OC)c(F)c4F)cnc23)ccc1C(=O)NCC(C)(C)CN ; Cl] | 509.4 | Intermediate 86 and tert-butyl (3- amino-2,2-di- methylpropyl)carba- mate |
| REF 201 | N-(1-amino-2- methylpropan-2-yl)- 4-((3-(2,3-difluoro- 4-methoxy- phenyl)imida- zo[1,2-a]pyrazin-8- yl)amino)-2- ethylbenzamide hydrochloride | 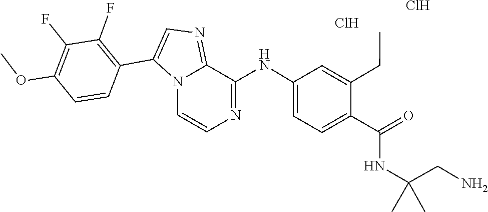 [SMILES: Cl ; CCc1cc(Nc2nccn3c(-c4ccc(OC)c(F)c4F)cnc23)ccc1C(=O)NC(C)(C)CN ; Cl] | 495.4 | Intermediate 86 and tert-butyl (2- amino-2- methylpropyl)carba- mate hydrochloride |
| REF 202 | N-(4-amino-2- methylbutan-2-yl)- 2-((3-(4- (difluoromethoxy)- 2,3-difluoro- phenyl)imida- zo[1,2-a]pyrazin-8- yl)amino)-2- m\ethylbenzamide hydrochloride | 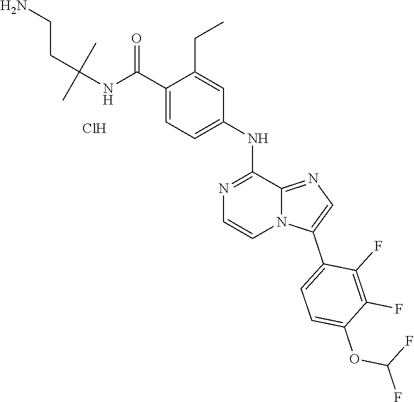 [SMILES: CCc1cc(Nc2nccn3c(-c4ccc(OC(F)F)c(F)c4F)cnc23)ccc1C(=O)NC(C)(C)CCN ; Cl] | 545.2 | Intermediate 85 and tert-butyl (3- amino-3- methyl- butyl)carbamate |
| REF 203 | N-(1-(amino- methyl)cyclo- propyl)-4-((3-(2,3- difluoro-4- methoxy- phenyl)imida- zo[1,2-a]pyrazin-8- yl)amino)-2- ethylbenzamide hydrochloride | 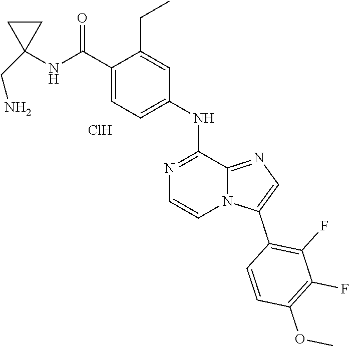 [SMILES: CCc1cc(Nc2nccn3c(-c4ccc(OC)c(F)c4F)cnc23)ccc1C(=O)NC1(CN)CC1 ; Cl] | 493.3 | Intermediate 86 and tert-butyl ((1- aminocyclo- propyl)meth- yl)carbamate |
| REF 204 | (R)-N-(1- aminopropan-2-yl)- 4-((3-(2,3-difluoro- 4-methoxy- phenyl)imida- zo[1,2-a]pyrazin-8- yl)amino)-2- ethylbenzamide dihydrochloride | 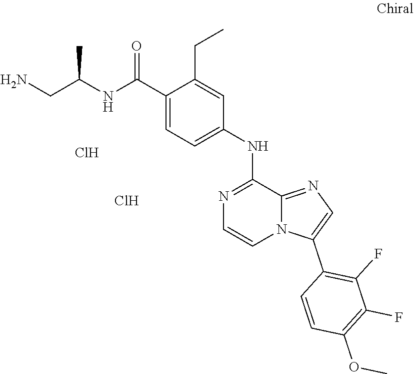 [SMILES: CCc1cc(Nc2nccn3c(-c4ccc(OC)c(F)c4F)cnc23)ccc1C(=O)N[C@H](C)CN ; Cl ; Cl] | 481.3 | Intermediate 86 and tert-butyl (R)-(2- aminopropyl)carba- mate |
| REF 205 | (S)-N-(1- aminopropan-2-yl)- 4-((3-(2,3-difluoro- 4-methoxy- phenyl)imida- zo[1,2-a]pyrazin-8- yl)amino)-2- ethylbenzamide dihydrochloride | 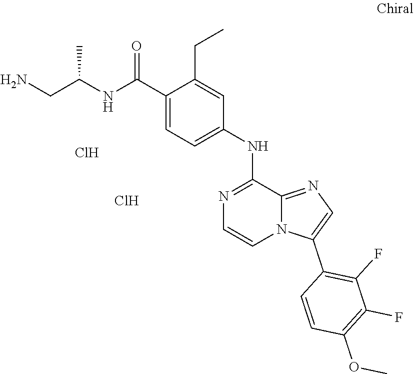 [SMILES: CCc1cc(Nc2nccn3c(-c4ccc(OC)c(F)c4F)cnc23)ccc1C(=O)N[C@@H](C)CN ; Cl ; Cl] | 481.3 | Intermediate 86 and tert-butyl (S)-(2- aminopropyl)carba- mate |
| REF 206 | N-(3-(2- aminoacetamido) propyl)-4-((3-(2,3- difluoro-4-methoxy- phenyl)imida- zo[1,2-a]pyrazin-8- yl)amino)-2- ethylbenzamide hydrochloride | 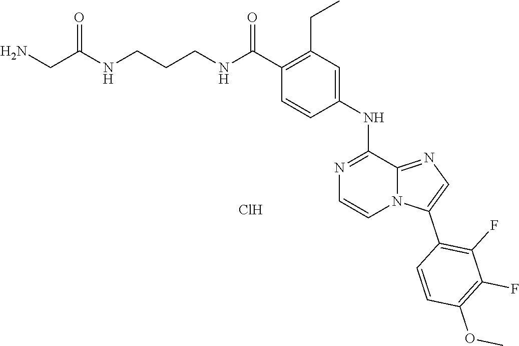 [SMILES: CCc1cc(Nc2nccn3c(-c4ccc(OC)c(F)c4F)cnc23)ccc1C(=O)NCCCNC(=O)CN ; Cl] | 538.3 | Intermediate 87 and (tert- butoxycarbo- nyl)glycine |
| REF 207 | (S)-(4-(4-((3-(2,3- difluoro-4- methoxy- phenyl)imida- zo[1,2-a]pyrazin-8- yl)amino)-2- methylbenzoyl)pi- perazin-1-yl)(4,4- dimethylpyrrolidin- 2-yl)methanone hydrochloride | 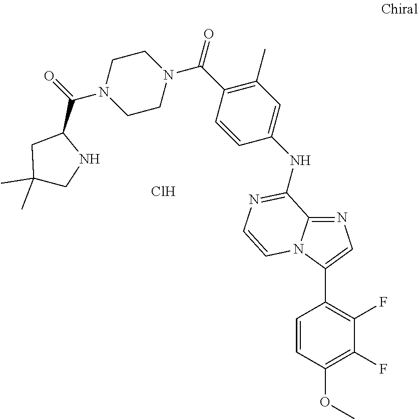 [SMILES: COc1ccc(-c2cnc3c(Nc4ccc(C(=O)N5CCN(C(=O)[C@@H]6CC(C)(C)CN6)CC5)c(C)c4)nccn23)c(F)c1F ; Cl] | 604.4 | Intermediate REF 208 and (S)-1- (tert- butoxycarbonyl)-4,4- dimethylpyrrolidine- 2-carboxylic acid |
| REF 209 | (S)-(4-(4-((3-(2,3- difluoro-4- methoxy- phenyl)imida- zo[1,2-a]pyrazin-8- yl)amino)-2- methylbenzoyl)pi- perazin-1-yl)(5,5- dimethylpyrrolidin- 2-yl)methanone hydrochloride | 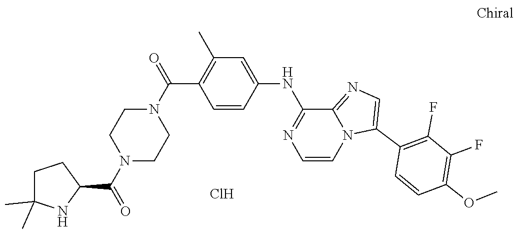 | 604.4 | Intermediate REF 208 and (S)-1- (tert- butoxycarbonyl)-5,5- dimethylpyrrolidine- 2-carboxylic acid |
| REF 210 | (4-(4-((3-(2,3- difluoro-4- methoxy- phenyl)imida- zo[1,2-a]pyrazin-8- yl)amino)-2- methylben- zoyl)piperazin- 1-yl)((2S,3R)- 3-hydroxy- pyrrolidin- 2-yl)methanone hydrochloride | 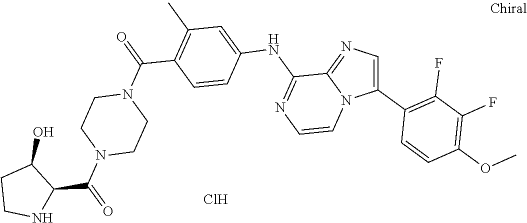 [SMILES: COc1ccc(-c2cnc3c(Nc4ccc(C(=O)N5CCN(C(=O)[C@H]6NCC[C@H]6O)CC5)c(C)c4)nccn23)c(F)c1F ; Cl] | 592.4 | Intermediate REF 208 and (2S,3R)-1-(tert- butoxycarbonyl)-3- hydroxypyrrolidine- 2-carboxylic acid |
| REF 211 | [4-[[3-(2,3-difluoro- 4-methoxy- phenyl)imida- zo[1,2-a]pyrazin-8- yl]amino]-2- methylphenyl]-[4- [(2R,3S)-3- hydroxypyrrolidine- 2- carbonyl]piperazin- 1- yl]methanone; hydrochloride | 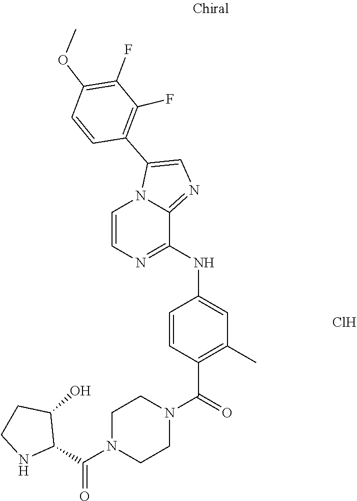 [SMILES: COc1ccc(-c2cnc3c(Nc4ccc(C(=O)N5CCN(C(=O)[C@@H]6NCC[C@@H]6O)CC5)c(C)c4)nccn23)c(F)c1F ; Cl] | 592.4 | Intermediate REF 208 and (2R,3S)-1-(tert- butoxycarbonyl)-3- hydroxypyrrolidine- 2-carboxylic acid |
| REF 212 | (4-(4-((3-(2,3- difluoro-4- methoxy- phenyl)imida- zo[1,2-a]pryazin-8- yl)amino)-2- methylbenzoyl)pi- perazin-1-yl)(2- methylpyrrolidin-2- yl)methanone hydrochloride | 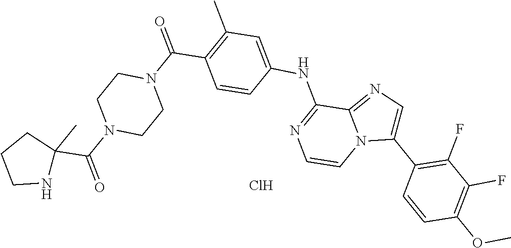 [SMILES: COc1ccc(-c2cnc3c(Nc4ccc(C(=O)N5CCN(C(=O)C6(C)CCCN6)CC5)c(C)c4)nccn23)c(F)c1F ; Cl] | 590.4 | Intermediate REF 208 and 1-(tert- butoxycarbonyl)-2- methylpyrrolidine-2- carboxylic acid |
| REF 213 | (4-(4-((3-(2,3- difluoro-4- methoxy- phenyl)imida- zo[1,2-a]pyrazin-8- yl)amino)-2- methyl- benzoyl)piper- azin-1-yl)((2S,4R)- 4-fluoropyrrolidin- 2-yl)methanone hydrochloride | 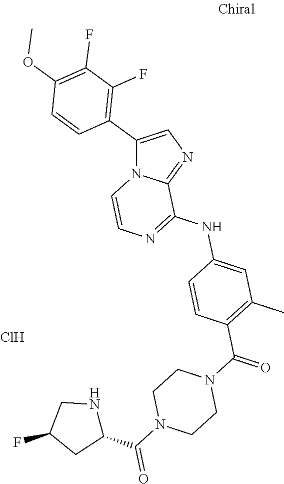 [SMILES: Cl ; COc1ccc(-c2cnc3c(Nc4ccc(C(=O)N5CCN(C(=O)[C@@H]6C[C@@H](F)CN6)CC5)c(C)c4)nccn23)c(F)c1F] | 594.4 | Intermediate REF 208 and (2S,4R)-1-(tert- butoxycarbonyl)-4- fluoropyrrolidine-2- carboxylic acid |
| REF 214 | (4-((1R,2S,5S)-3- azabicyclo[3.1.0] hexane-2- carbonyl)piperazin- 1-yl)(4-((3-(2,3- difluoro-4- methoxy- phenyl)imida- zo[1,2-a]pyrazin-8- yl)amino)-2- methylphe- nyl)methanone hydrochloride | 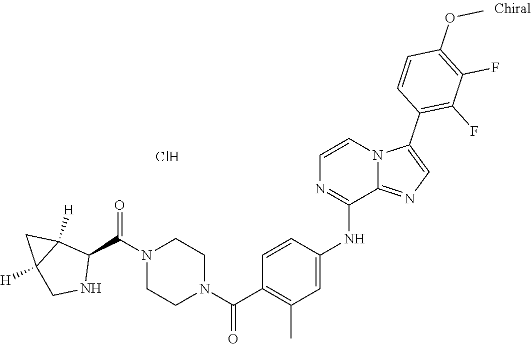 [SMILES: COc1ccc(-c2cnc3c(Nc4ccc(C(=O)N5CCN(C(=O)[C@H]6NC[C@H]7C[C@H]76)CC5)c(C)c4)nccn23)c(F)c1F ; Cl] | 588.4 | Intermediate REF 208 and rac- (1R,2S,5S)-3-(tert- butoxycarbonyl)-3- azabicyclo[3.1.0]hex- ane-2-carboxylic acid |
| REF 215 | (4-(4-((3-(2,3- difluoro-4- methoxy- phenyl)imida- zo[1,2-a]pyrazin-8- yl)amino)-2- methyl- benzoyl)piper- azin-1-yl)((2S,4S)- 4-fluoropyrrolidin- 2-yl)methanone hydrochloride | 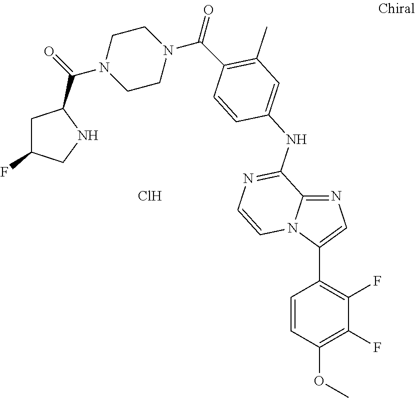 [SMILES: COc1ccc(-c2cnc3c(Nc4ccc(C(=O)N5CCN(C(=O)[C@@H]6C[C@H](F)CN6)CC5)c(C)c4)nccn23)c(F)c1F ; Cl] | 594.4 | Intermediate REF 208 and (2S,4S)-1-(tert- butoxycarbonyl)-4- fluoropyrrolidine-2- carboxylic acid |
| REF 216 | (4-(4-((3-(2,3- difluoro-4- methoxy- phenyl)imida- zo[1,2-a]pyrazin-8- yl)amino)-2-methyl- benzoyl)piperazin- 1-yl)((2S,4S)-4- (methoxymethyl) pyrrolidin-2- yl)methanone hydrochloride | 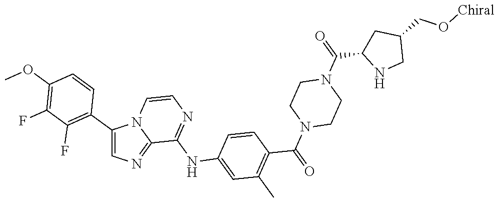 | 620.4 | Intermediate REF 208 and (2S,4S)-1-(tert- butoxycarbonyl)-4- (methoxymethyl)pyr- rolidine-2- carboxylic acid |
| REF 217 | [4-[(2S,4R)-4- aminopyrrolidine-2- carbonyl]piperazin- 1-yl]-[4-[[3- (2,3difluoro-4- methoxy- phenyl)imida- zo[1,2-a]pyrazin-8- yl]amino]-2- methylphe- nyl]methanone | 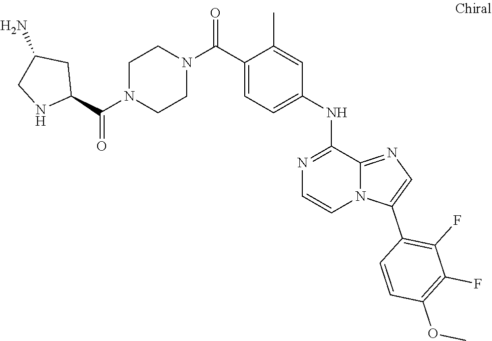 [SMILES: COc1ccc(-c2cnc3c(Nc4ccc(C(=O)N5CCN(C(=O)[C@@H]6C[C@@H](N)CN6)CC5)c(C)c4)nccn23)c(F)c1F] | 591.4 | Intermediate REF 208 and (2S,4R)-4-((((9H- fluoren-9- yl)methoxy)carbonyl) amino)-1-(tert- butoxycarbonyl)pyrro- lidine-2-carboxylic acid. Final Fmoc removal with 4- methylpiperidine |
| REF 218 | (4-amniopiperidin- 4-yl)(4-(4-((3-(2,3- difluoro-4-methoxy- phenyl)imida- zo[1,2-a]pyrazin-8- yl)amino)-2- methyl- benzoyl)piper- azin-1-yl)metha- none | 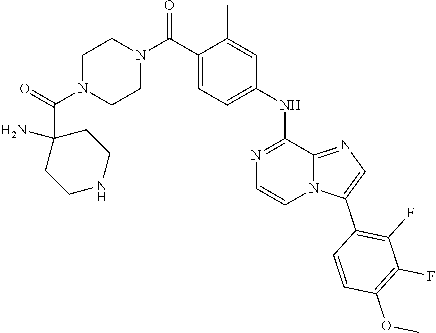 [SMILES: COc1ccc(-c2cnc3c(Nc4ccc(C(=O)N5CCN(C(=O)C6(N)CCNCC6)CC5)c(C)c4)nccn23)c(F)c1F] | 605.3 | Intermediate REF 208 and 4- ((((9H-fluoren-9- yl)methoxy)carbonyl) amino)-1-(tert- butoxycarbonyl)piper- idine-4-carboxylic acid. Final Fmoc removal with piperidine |
| REF 219 | 2-(4-(8-((4-(4-(2,6- diazaspiro[3.3]hep- tane-2- carbonyl)piperidine- 1-carbonyl)-3- methyl- phenyl)amino) imidazo[1,2- a]pyrazin-3-yl)-2,3- difluorophenoxy) acetonitrile | 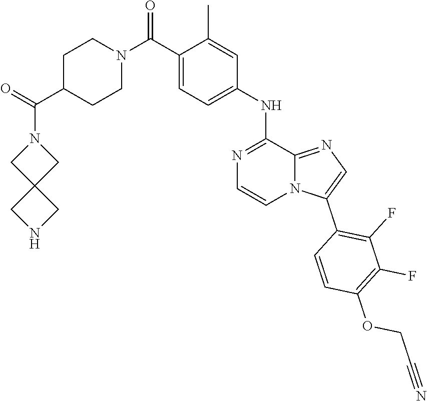 [SMILES: Cc1cc(Nc2nccn3c(-c4ccc(OCC#N)c(F)c4F)cnc23)ccc1C(=O)N1CCC(C(=O)N2CC3(CNC3)C2)CC1] | 625.4 (M − H) | Intermeidate 69 and tert-butyl 2,6- diazaspiro[3.3]hep- tane-2-carboxylate |
| REF 220 | 1-(4-((3-(2,3- difluoro-4-methoxy- phenyl)imida- zo[1,2-a]pyrazin-8- yl)amino)-2- methylbenzoyl)-N- (2-hydroxy-3- ureidopropyl)piperi- dine-4-carboxamide | 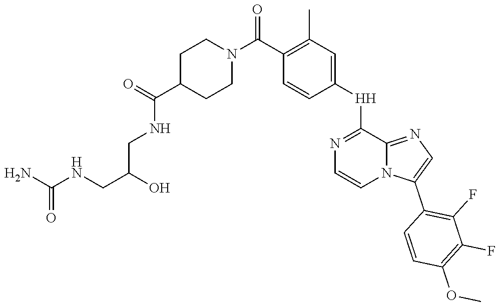 | 637.3 | Intermediate 65 and 1-(3-amino-2- hydroxypropyl)urea |
| REF 221 | 1-(4-((3-(2,3- difluoro-4-methoxy- phenyl)imida- zo[1,2-a]pyrazin-8- yl)amino)-2- methylbenzoyl)-N- (3-(dimethylamino)- 2-hydroxy- propyl)piperi- dine-4-carboxamide | 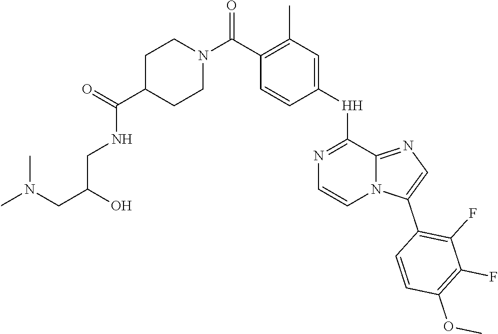 | 622.2 | Intermediate 65 and 1-amino-3- (dimethylamino)pro- pan-2-ol |
| REF 222 | 1-(4-((3-(2,3- difluoro-4-methoxy- phenyl)imida- zo[1,2-a]pyrazin-8- yl)amino)-2- methylbenzoyl)-N- (2-hydroxy-3- (piperazin-1- yl)propyl)piperidine- 4-carboxamide hydrochloride | 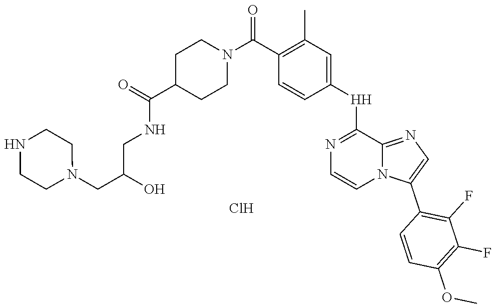 | 663.3 | Intermediate 65 and tert-butyl 4-(3- amino-2-hydroxy- propyl)pipera- zine-1-carboxylate |

### Reference Example 223 and Reference Example 224

### 2-chloro-4-[[3-[4-(cyanomethoxy)-2,3-difluoro-phenyl]imidazo[1,2-a]pyrazin-8-yl]amino]-N-[3-(dimethylamino)propylcarbamoyl]-N-ethyl-benzamide and 2-chloro-4-[[3-[4-(cyanomethoxy)-2,3-difluoro-phenyl]imidazo[1,2-a]pyrazin-8-yl]amino]-N-[2-(1H-tetrazol-5-yl)ethyl]benzamide

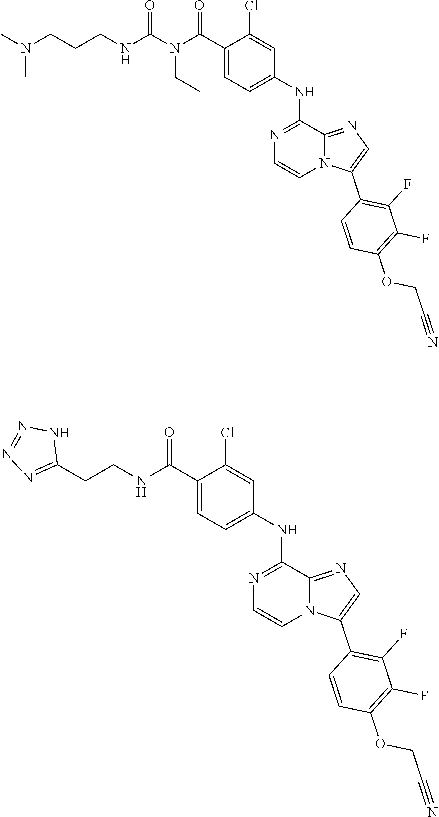 [SMILES: CCN(C(=O)NCCCN(C)C)C(=O)c1ccc(Nc2nccn3c(-c4ccc(OCC#N)c(F)c4F)cnc23)cc1Cl ; N#CCOc1ccc(-c2cnc3c(Nc4ccc(C(=O)NCCc5nnn[nH]5)c(Cl)c4)nccn23)c(F)c1F]

To a solution of Intermediate 29 (230 mg, 0.5 mmol), 4-(4-methyl-4H-1,2,4-triazol-3-yl)piperidine hydrochloride (122 mg, 0.6 mmol) in anhydrous DMF (10 mL) was added DIPEA (129 mg, 1.0 mmol) and DMAP (73 mg, 0.6 mmol), Followed by the resultant mixture was stirred for 30 min at room temperature, EDCI (115 mg, 0.6 mmol) was added in the mixture and stirred for extra 10 h.

The mixture was poured into water (50 mL) and the aqueous solution was extracted with DCM (50 mL×2). The organic layers were combined and washed with water and brine, dried over anhydrous sodium sulphate and concentrated under reduced pressure to give a red oil, which was purified by prep.HPLC to give Reference Example 223 (50 mg, 16% yield) as a white powder MS (ESI, m/z): 611.2 [M+H]+ and Reference Example 224 (60 mg, 21.3% yield) as a white powder. MS (ESI, m/z): 551.1 [M+H]+

### Reference Example 225

### N-[[1-[4-[[3-[4-(cyanomethoxy)-2,3-difluoro-phenyl]imidazo[1,2-a]pyrazin-8-yl]amino]-2-methyl-benzoyl]-4-piperidyl]methyl]-2-hydroxy-acetamide

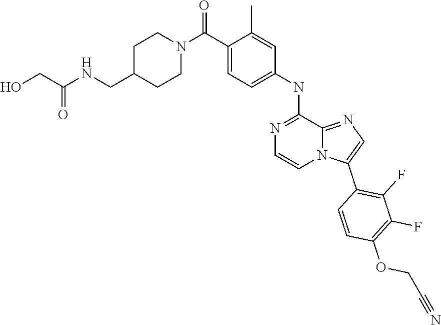 [SMILES: Cc1cc(Nc2nccn3c(-c4ccc(OCC#N)c(F)c4F)cnc23)ccc1C(=O)N1CCC(CNC(=O)CO)CC1]

To a solution of Reference Example 129 (106 mg, 0.2 mmol), 2-hydroxyacetic acid (16 mg, 0.2 mmol) in anhydrous DMF (5 mL) was added DIPEA (52 mg, 0.4 mmol) and then the resultant mixture was stirred for 30 min at room temperature, HATU (152 mg, 0.4 mmol) was added in the mixture and stirred for extra 10 h. The mixture was poured into water (50 mL) and the aqueous solution was extracted with DCM (50 mL×2). The organic layers were combined and washed with water and brine, dried over anhydrous sodium sulphate and concentrated under reduced pressure to give a red oil, which was purified by prep.HPLC to give Reference Example 225 (5 mg, 4.2% yield) as a white powder MS (ESI, m/z): 590.2 [M+H]+

The following example was prepared in analogy to Reference Example 225

| | | | ESI MS | |
|---|---|---|---|---|
| Ex. | Name | Structure | [M + H]⁺ | Starting Material |
| 29 | N-[[1-[2-chloro-4-[[3-[4- (cyanomethoxy)-2,3- difluoro- phenyl]imidazo[1,2- a]pyrazin-8- yl]amino]benzoyl]-4- piperidyl]methyl]pyridine- 4-carboxamide 2,2,2- trifluoroacetate | 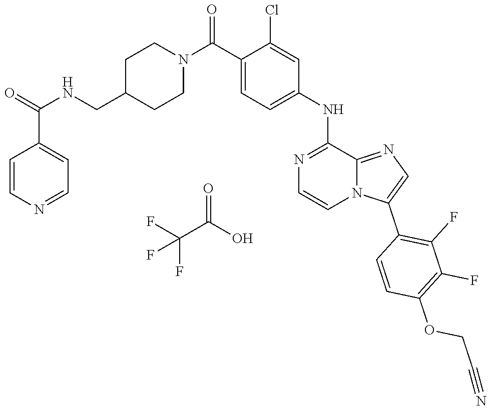 [SMILES: N#CCOc1ccc(-c2cnc3c(Nc4ccc(C(=O)N5CCC(CNC(=O)c6ccncc6)CC5)c(Cl)c4)nccn23)c(F)c1F ; O=C(O)C(F)(F)F] | 657.1 | Example 12 and isonicotinic acid |

### Intermediate 81

### tert-butyl 2-(hydroxymethyl)-4-(piperidine-4-carbonyl)piperazine-1-carboxylate

### Step 1: tert-butyl 4-(1-benzyloxycarbonylpiperidine-4-carbonyl)-2-(hydroxymethyl)piperazine-1-carboxylate

A mixture of 1-[(benzyloxy)carbonyl]piperidine-4-carboxylic acid (2.89 g, 10.99 mmol, 1.1 eq), tert-butyl 2-(hydroxymethyl)piperazine-1-carboxylate (2.16 g, 9.99 mmol, 1 eq), 0-(7-azabenzotriazol-1-yl)-N,N,N′,N′-tetramethyluronium hexafluorophosphate (5.7 g, 14.98 mmol, 1.5 eq) and N,N-diisopropylethylamine (5.22 mL, 29.96 mmol, 3 eq) in DMF (25 mL) was stirred at 25° C. for 14 h. The mixture was added water (50 mL) and extracted with ethyl acetate (50 mL×3). The combined organic layers were washed with saturated NH₄Cl solution (50 mL) and concentrated to dryness. The crude product was purified by prep. HPLC. To the desired fractions were added NaHCO₃ (s) until pH 7˜8 and extracted with ethyl acetate (100 mL×3). The combined organic layers were dried over sodium sulphate and concentrated in vacuo to afford tert-butyl 4-(1-benzyloxycarbonylpiperidine-4-carbonyl)-2-(hydroxymethyl)piperazine-1-carboxylate (2.28 g) as a brown oil. MS obsd. (ESI⁺): 461.9 [(M+H)⁺].

### Step 2: tert-butyl 2-(hydroxymethyl)-4-(piperidine-4-carbonyl)piperazine-1-carboxylate

A mixture of tert-butyl 4-(1-benzyloxycarbonylpiperidine-4-carbonyl)-2-(hydroxymethyl)piperazine-1-carboxylate (2.08 g, 4.51 mmol, 1 eq) and Pd/C (10%, 300 mg) in ethyl acetate (20 mL) was stirred at 25° C. for 72 h under hydrogen atmosphere. The mixture was filtered over celite and the filtrate was concentrated to dryness to afford tert-butyl 2-(hydroxymethyl)-4-(piperidine-4-carbonyl)piperazine-1-carboxylate (Intermediate 81) (1.29 g, 3.94 mmol, 87.43% yield) as black oil. The crude product was used in next step without any purification.

MS obsd. (ESI⁺): 328.2 [(M+H)⁺].

### Reference Example 226

### 4-[[3-(3-fluoro-4-methoxy-phenyl)imidazo[1,2-a]pyrazin-8-yl]amino]-N-(2-imidazol-1-ylethyl)-N,2-dimethyl-benzamide

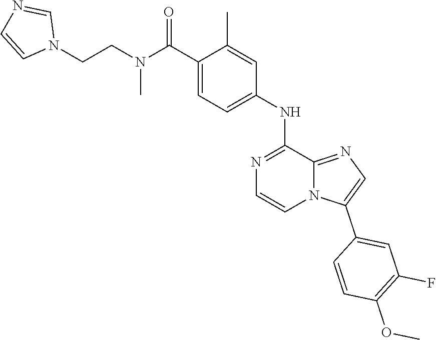 [SMILES: COc1ccc(-c2cnc3c(Nc4ccc(C(=O)N(C)CCn5ccnc5)c(C)c4)nccn23)cc1F]

### Step 1: 2-imidazol-1-ylethyl Methanesulfonate

A mixture of 1-(2-hydroxyethyl)imidazole (1.0 g, 8.92 mmol) in DCM (10 mL) was added methanesulfonyl chloride (1.02 g, 8.92 mmol) and triethylamine (2.5 mL, 17.84 mmol). After stirring at 20° C. for 4 h, the reaction was quenched with H₂O (10 mL) and concentrated to dryness. The residue was diluted with EA (30 mL) and washed with water (30 mL) and brine (30 mL), dried over anhydrous sodium sulphate, concentrated under reduced pressure to afford the crude title product (500 mg) as yellow oil which was used in next step directly.

### Step 2: 2-imidazol-1-yl-N-methyl-ethanamine

A mixture of 2-imidazol-1-ylethyl methanesulfonate (250 mg, 1.31 mmol) and a solution of monomethylamine in EtOH (2 mL) was stirred at 70° C. for 12 h. The reaction mixture was concentrated under reduced pressure to afford crude product (150 mg) as yellow oil which was used directly in next step.

### Step 3: 4-[[3-(3-fluoro-4-methoxy-phenyl)imidazo[1,2-a]pyrazin-8-yl]amino]-N-(2-imidazol-1-ylethyl)-N,2-dimethyl-benzamide

Into a stirred solution of intermediate 6 (200 mg, 0.51 mmol), 2-imidazol-1-yl-N-methyl-ethanamine (96 mg, 0.76 mmol) and triethylamine (0.2 mL, 1.53 mmol) in DMF (2 mL) was added 1-propanephosphonic anhydride (486 mg, 0.76 mmol) slowly. The reaction was stirred at 25° C. for 12 h and then concentrated to dryness. The residue was diluted with ethyl acetate (10 mL) and the resulting mixture was washed with water (3 mL) and brine (3 mL), dried over anhydrous sodium sulphate, concentrated under reduced pressure to afford crude product. The residue was purified by prep-HPLC to afford the title compound (50 mg) as yellow solid. MS obsd. (ESI⁺) [(M+H)⁺]: 500.3

### Reference Example 227

### 2-chloro-4-[[3-(2,3-difluoro-4-methoxy-phenyl)imidazo[1,2-a]pyrazin-8-yl]amino]-N-[(1,1-dioxothian-4-yl)methyl]benzamide;2,2,2-trifluoroacetic Acid

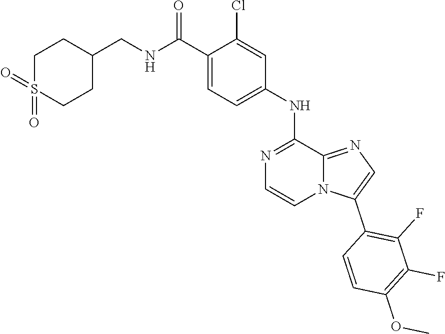 [SMILES: COc1ccc(-c2cnc3c(Nc4ccc(C(=O)NCC5CCS(=O)(=O)CC5)c(Cl)c4)nccn23)c(F)c1F]

A mixture of Reference Example 80 (200 mg, 368 μmol) and oxone (678 mg, 1.1 mmol) in DMF (5 mL) was stirred at rt for 4 hrs. The mixture was diluted with H₂O (40 mL) and extracted with DCM. The organic layer was dried and concentrated in vacuo. The residue was purified by prep-HPLC to give the title compound (56 mg) as light yellow solid. MS (ESI, m/z): 576.1.

### Reference Example 228

### N-[2-(2-Aminoethoxy)ethyl]-2-methyl-4-[[3-(4-prop-2-ynoxyphenyl)imidazo[1,2-a]pyrazin-8-yl]amino]benzamide; Formic Acid

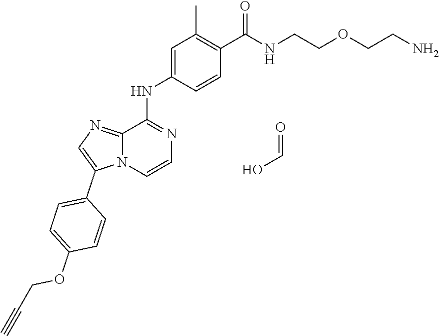 [SMILES: C#CCOc1ccc(-c2cnc3c(Nc4ccc(C(=O)NCCOCCN)c(C)c4)nccn23)cc1 ; O=CO]

### Step 1

### 8-Chloro-3-(4-prop-2-ynoxyphenyl)imidazo[1,2-a]pyrazine

A mixture of 4-(8-chloroimidazo[1,2-a]pyrazin-3-yl)phenol (intermediate 50, 100 mg, 0.410 mmol, 1 eq) and potassium carbonate (169 mg, 1.22 mmol, 3 eq) in DMF (3 mL) was added propargyl bromide (145 mg, 1.22 mmol, 3 eq) at 20° C. and stirred at 20° C. for 16 h. The mixture was filtered, poured into water, extracted with ethyl acetate, concentrated and purified by prep-TLC (PE/ethyl acetate=1:1) to afford the desired product (61 mg) as a yellow solid.

### Step 2

### tert-Butyl N-[2-[2-[[2-methyl-4-[[3-(4-prop-2-ynoxyphenyl)imidazo[1,2-a]pyrazin-8-yl]amino]benzoyl]amino]ethoxy]ethyl]carbamate

A mixture of tert-butyl N-[2-[2-[(4-amino-2-methyl-benzoyl)amino]ethoxy]ethyl]carbamate (intermediate 78, 45.0 mg, 0.130 mmol, 1 eq), 8-chloro-3-(4-prop-2-ynoxyphenyl)imidazo[1,2-a]pyrazine (37.84 mg, 0.130 mmol, 1 eq), cesium carbonate (130.36 mg, 0.400 mmol, 3 eq), tris(dibenzylideneacetone)dipalladium (0) (12.21 mg, 0.010 mmol, 0.100 eq) and 9,9-dimethyl-4,5-bis(diphenylphosphino)xanthene (7.72 mg, 0.010 mmol, 0.100 eq) in 1,4-dioxane (5 mL) was stirred under N₂ at 115° C. on microwave for 2 h. The mixture was filtered and concentrated, purified by prep-TLC (DCM/MeOH/MeCN=10:1:1) to afford product (20 mg) as a light yellow solid.

### Step 3

### N-[2-(2-Aminoethoxy)ethyl]-2-methyl-4-[[3-(4-prop-2-ynoxyphenyl)imidazo[1,2-a]pyrazin-8-yl]amino]benzamide Formate

A solution of tert-butyl N-[2-[2-[[2-methyl-4-[[3-(4-prop-2-ynoxyphenyl)imidazo[1,2-a]pyrazin-8-yl]amino]benzoyl]amino]ethoxy]ethyl]carbamate (50.0 mg, 0.090 mmol, 1 eq) in DCM (4 mL) was added trifluoroacetic acid (0.39 mL, 5.11 mmol, 59.78 eq) and stirred at 20° C. for 16 h. The solution was concentrated and purified by prep-HPLC to afford 13.4 mg product as white solid. MS (ESI, m/z): 485.4

### Reference Example 229

### N-[2-(2-aminoethoxy)ethyl]-4-[[3-[4-(4-hydroxybut-2-ynoxy)phenyl]imidazo[1,2-a]pyrazin-8-yl]amino]-2-methyl-benzamide Formate

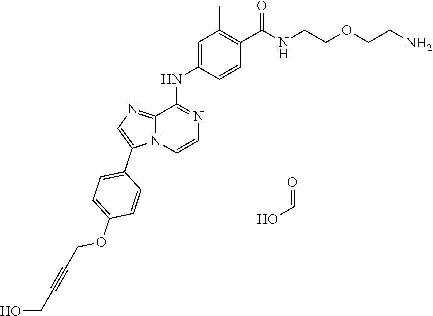 [SMILES: Cc1cc(Nc2nccn3c(-c4ccc(OCC#CCO)cc4)cnc23)ccc1C(=O)NCCOCCN ; O=CO]

The title compound was obtained in analogy to Reference Example 228 using 4-hydroxybut-2-ynyl methanesulfonate instead of propargyl bromide. MS (ESI, m/z): 515.3

### Reference Example 230

### N-(2-(2-aminoethoxy)ethyl)-4-((3-(3-chloro-4-methoxyphenyl)imidazo[1,2-a]pyrazin-8-yl)amino)-2-methylbenzamide Hydrochloride

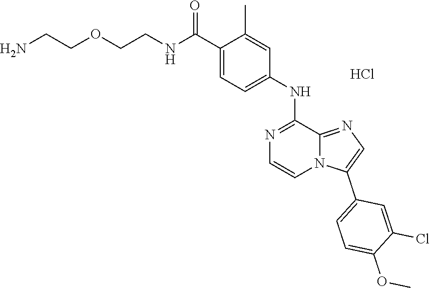 [SMILES: COc1ccc(-c2cnc3c(Nc4ccc(C(=O)NCCOCCN)c(C)c4)nccn23)cc1Cl ; Cl]

To Intermediate 79 (24 mg) in dioxane (900 μl) and water (100 μl) was added (3-chloro-4-methoxyphenyl)boronic acid (11.6 mg), 1,1′-bis(diphenylphosphino)ferrocenedichloro palladium(II) dichloromethane complex (3.03 mg, 4.13 μmol) and potassium carbonate (14.3 mg, 103 μmol) followed by stirring at 105° C. overnight. The reaction mixture was concentrated and purified by prep. HPLC to give a Boc-protected intermediate, which was deprotected to the title compound (11 mg, colorless solid) by addition of 4M HCl in dioxane (1 h), followed by concentration and drying in vacuo. MS (ESI, m/z): 495.3

The following examples were prepared in analogy to Reference Example 230:

| | | | MS | |
|---|---|---|---|---|
| | | | ESI | |
| Ex. | Name | Structure | [M + H]⁺ | Starting Material |
| REF 231 | N-(2-(2- aminoethoxy)ethyl)- 4-((3-(4- fluorophenyl)imidazo [1,2-a]pyrazin-8- yl)amino)-2- methylbenzamide hydrochloride | 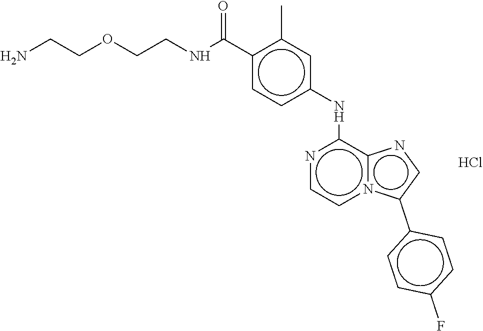 | 449.7 | Intermediate 79 and (4- fluorophenyl) boronic acid |
| REF 232 | N-(2-(2- aminoethoxy)ethyl)- 4-((3-(4-chloro-3- fluorophenyl)imidazo [1,2-a]pyrazin-8- yl)amino)-2- methylbenzamide hydrochloride | 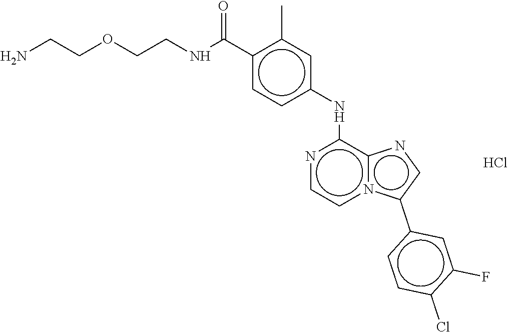 | 483.1 | Intermediate 79 and (4- chloro-3- fluorophenyl) boronic acid |
| REF 233 | N-(2-(2- aminoethoxy)ethyl)- 4-((3-(3,4- difluorophenyl)imidazo [1,2-a]pyrazin-8- yl)amino)-2- methylbenzamide hydrochloride | 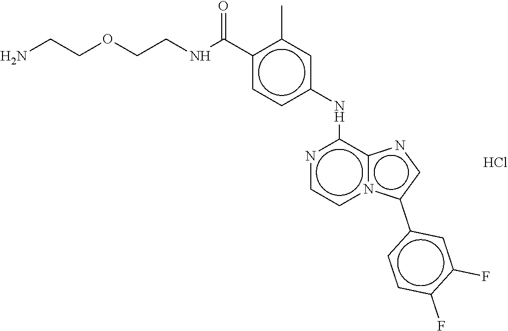 | 467.9 | Intermediate 79 and (3,4- difluorophenyl) boronic acid |
| REF 234 | N-(2-(2- aminoethoxy)ethyl)- 4-((3-(2-fluoro-4- methoxyphenyl)imidazo [1,2-a]pyrazin-8- yl)amino)-2- methylbenzamide hydrochloride | 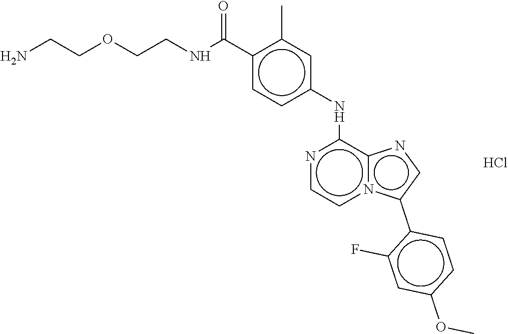 | 479.4 | Intermediate 79 and (2- fluoro-4- methoxyphenyl) boronic acid |
| REF 235 | N-(2-(2- aminoethoxy)ethyl)- 4-((3-(2,4- difluorophenyl)imidazo [1,2-a]pyrazin-8- yl)amino)-2- methylbenzamide hydrochloride | 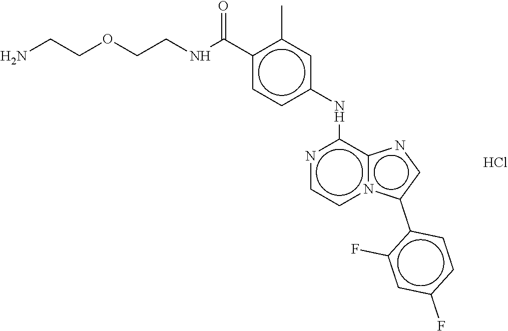 | 467.2 | Intermediate 79 and (2,4- difluorophenyl) boronic acid |
| REF 236 | N-(2-(2- aminoethoxy)ethyl)- 4-((3-(3-fluoro-4- methoxyphenyl)imidazo [1,2-a]pyrazin-8- yl)amino)-2- methylbenzamide hydrochloride | 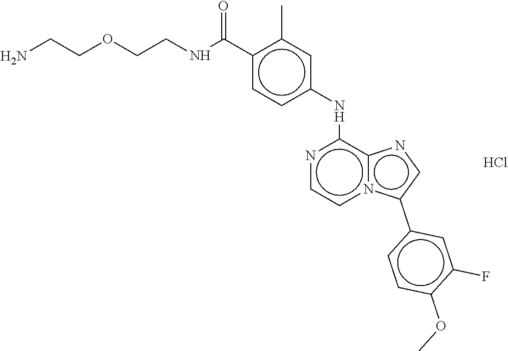 | 479.1 | Intermediate 79 and (3- fluoro-4- methoxyphenyl) boronic acid |
| REF 237 | N-(2-(2- aminoethoxy)ethyl)- 2-methyl-4-((3- (2,3,4- trifluorophenyl)imidazo [1,2-a]pyrazin-8- yl)amino)benzamide hydrochloride | 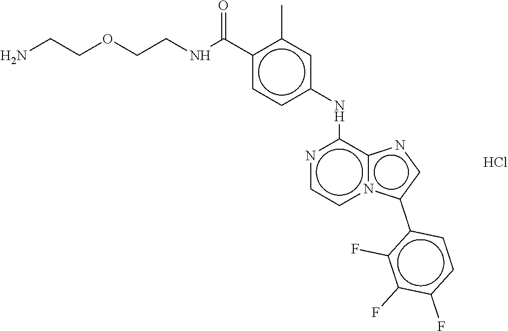 | 485.2 | Intermediate 79 and (2,3,4- trifluorophenyl) boronic acid |
| REF 238 | (4-(4-((3-(3-chloro- 4- methoxyphenyl)imidazo [1,2-a]pyrazin-8- yl)amino)-2- methylbenzoyl)piperazin- 1-yl)((2S,4R)- 4-hydroxypyrrolidin- 2-yl)methanone hydrochloride | 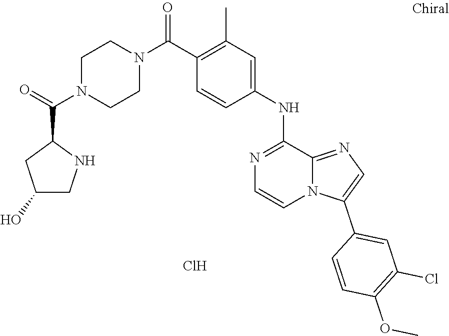 [SMILES: COc1ccc(-c2cnc3c(Nc4ccc(C(=O)N5CCN(C(=O)[C@@H]6C[C@@H](O)CN6)CC5)c(C)c4)nccn23)cc1Cl ; Cl] | 590.2 | Intermediate 75 and (3-chloro-4- methoxyphenyl) boronic acid |
| REF 239 | (4-(4-((3-(3-chloro- 2-fluoro-4- methoxyphenyl)imidazo [1,2-a]pyrazin-8- yl)amino)-2- methylbenzoyl)piperazin- 1-yl)((2S,4R)- 4-hydroxypyrrolidin- 2-yl)methanone hydrochloride | 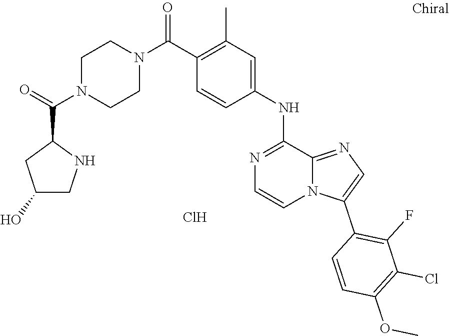 [SMILES: COc1ccc(-c2cnc3c(Nc4ccc(C(=O)N5CCN(C(=O)[C@@H]6C[C@@H](O)CN6)CC5)c(C)c4)nccn23)c(F)c1Cl ; Cl] | 609.1 | Intermediate 75 and (3-chloro- 2-fluoro- 4- methoxyphenyl) boronic acid |
| REF 240 | 2-(2-chloro-4-(8-((4- (4-((2S,4R)-4- hydroxypyrrolidin-2- carbonyl)piperazine- 1-carbonyl)-3- methylphenyl)amino) imidazo[1,2- a]pyrazin-3- yl)phenoxy)acetonitrile | 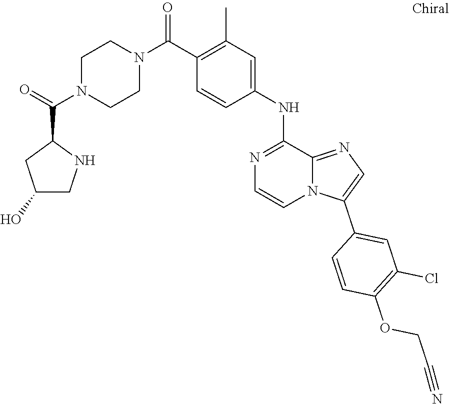 [SMILES: Cc1cc(Nc2nccn3c(-c4ccc(OCC#N)c(Cl)c4)cnc23)ccc1C(=O)N1CCN(C(=O)[C@@H]2C[C@@H](O)CN2)CC1] | 615.2 | Intermediate 75 and (2-(2-chloro-4- (4,4,5,5- tetramethyl- 1,3,2- dioxaborolan-2- yl)phenoxy) acetonitrile (Intermediate 88) |

### Reference Example 241

### 4-[[3-(2,3-difluoro-4-methoxy-phenyl)imidazo[1,2-a]pyrazin-8-yl]amino]-N,N-dimethyl-benzamide

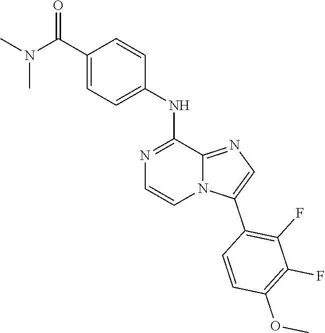 [SMILES: COc1ccc(-c2cnc3c(Nc4ccc(C(=O)N(C)C)cc4)nccn23)c(F)c1F]

A mixture of 4-((3-iodoimidazo[1,2-a]pyrazin-8-yl)amino)-N,N-dimethylbenzamide (150 mg, 368 μmol), (2,3-difluoro-4-methoxyphenyl)boronic acid (69.2 mg, 368 μmol), K₃PO₄ (235 mg, 1.11 mmol) and PdCl₂(dppf)-CH₂Cl₂ adduct (13.5 mg, 18.4 μmol) in THF (5 mL) and H₂O (1 mL) was heated to 50° C. with stirring overnight. The reaction mixture was diluted with H₂O and extracted with DCM (30 mL) twice. The combined DCM layer was dried and concentrated in vacuo. The residue was purified by prep-HPLC to give 4-((3-(2,3-difluoro-4-methoxyphenyl)imidazo[1,2-a]pyrazin-8-yl)amino)-N,N-dimethylbenzamide (20 mg) as white solid. (ESI+) [(M+H)⁺]: 0.424.

### Reference Example 242

### N-[2-[2-[(dimethylamino)methyl]morpholin-4-yl]ethyl]-4-[[3-(3-fluoro-4-methoxy-phenyl)imidazo[1,2-a]pyrazin-8-yl]amino]-N,2-dimethyl-benzamide; Formic Acid

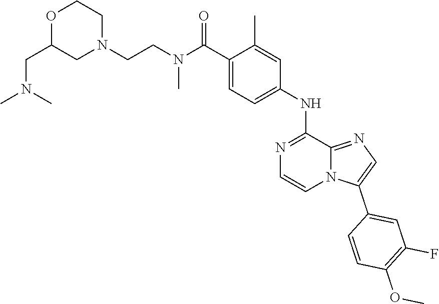 [SMILES: COc1ccc(-c2cnc3c(Nc4ccc(C(=O)N(C)CCN5CCOC(CN(C)C)C5)c(C)c4)nccn23)cc1F]

### Step 1: N-(2-chloroethyl)-N,2-dimethyl-4-nitro-benzamide

To a solution of (2-chloroethyl)methylamine (310 mg, 3.31 mmol), 2-methyl-4-nitro-benzoic acid (500 mg, 2.76 mmol), triethylamine (1.15 mL, 8.28 mmol) in DMF (8 mL) was added T3P (2.63 g, 4.14 mmol). The mixture was stirred at 20° C. for 16 h. The mixture was diluted with ethyl acetate (100 mL), washed with water (30 mL), brine (30 mL), dried over sodium sulphate and concentrated. The residue was purified by column chromatography to give N-(2-chloroethyl)-N,2-dimethyl-4-nitro-benzamide (480 mg) as a colorless oil.

### Step 2: N-[2-[2-[(dimethylamino)methyl]morpholin-4-yl]ethyl]-N,2-dimethyl-4-nitro-benzamide

A mixture of N,N-dimethyl-2-morpholinmethanamine (148 mg, 1.03 mmol), N-(2-chloroethyl)-N,2-dimethyl-4-nitro-benzamide (220 mg, 0.860 mmol), N,N-diisopropylethylamine (0.6 mL, 3.43 mmol) in DMSO (3 mL) was stirred at 100° C. for 16 h. After cooling to room temperature, the mixture was diluted with ethyl acetate (100 mL), washed with water (30 mL), brine (30 mL×2), dried over sodium sulphate and concentrated under reduced pressure. The residue was purified by prep-HPLC (TFA). The fraction was concentrated. The residue was neutralized by aq. NaHCO₃, extracted with ethyl acetate (100 mL), dried over sodium sulphate and concentrated to give N-[2-[2-[(dimethylamino)methyl]morpholin-4-yl]ethyl]-N,2-dimethyl-4-nitro-benzamide (60 mg) as a light-yellow oil.

### Step 3: 4-amino-N-[2-[2-[(dimethylamino)methyl]morpholin-4-yl]ethyl]-N,2-dimethyl-benzamide

To a solution of N-[2-[2-[(dimethylamino)methyl]morpholin-4-yl]ethyl]-N,2-dimethyl-4-nitro-benzamide (60 mg, 0.160 mmol) in ethyl acetate (3 mL) was added Pd/C (10%, 20 mg). The mixture was stirred at 20° C. under H₂ for 16 h. The mixture was filtered and the filtrate was concentrated under reduced pressure to give crude 4-amino-N-[2-[2-[(dimethylamino)methyl]morpholin-4-yl]ethyl]-N,2-dimethyl-benzamide (50 mg) as a yellow oil, which was used directly for the next step without further purification.

### Step 4: N-[2-[2-[(dimethylamino)methyl]morpholin-4-yl]ethyl]-4-[[3-(3-fluoro-4-methoxy-phenyl)imidazo[1,2-a]pyrazin-8-yl]amino]-N,2-dimethyl-benzamide; Formic Acid

A mixture of 8-chloro-3-(3-fluoro-4-methoxy-phenyl)imidazo[1,2-a]pyrazine (50 mg, 0.180 mmol, 3), 4-amino-N-[2-[2-[(dimethylamino)methyl]morpholin-4-yl]ethyl]-N,2-dimethyl-benzamide (50 mg, 0.150 mmol), Brettphos Pd G3 (14 mg, 0.020 mmol, CAS #1470372-59-8), potassium carbonate (62 mg, 0.450 mmol) in tert-butanol (1 mL) was stirred at 100° C. for 16 h. The mixture was diluted with ethyl acetate (100 mL), washed with water (30 mL), brine (30 mL), dried over sodium sulphate and concentrated under reduced pressure. The residue was purified by prep-TLC (DCM:MeOH=10:1), then further purified by prep-HPLC (FA) to give N-[2-[2-[(dimethylamino)methyl]morpholin-4-yl]ethyl]-4-[[3-(3-fluoro-4-methoxy-phenyl)imidazo[1,2-a]pyrazin-8-yl]amino]-N,2-dimethyl-benzamide (25.5 mg) as a white solid. MS obsd. (ESI⁺) [(M+H)⁺]: 576.2

### Reference Example 243

### 4-[[3-(3-fluoro-4-methoxy-phenyl)imidazo[1,2-a]pyrazin-8-yl]amino]-N,2-dimethyl-N-[2-[2-(methylaminomethyl)morpholin-4-yl]ethyl]benzamide Hydrochloride

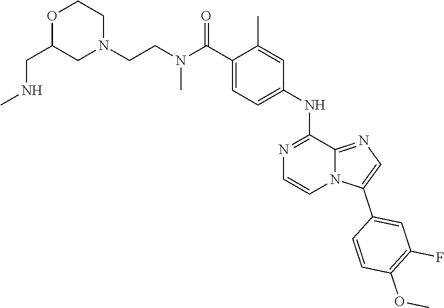 [SMILES: CNCC1CN(CCN(C)C(=O)c2ccc(Nc3nccn4c(-c5ccc(OC)c(F)c5)cnc34)cc2C)CCO1]

### Step 1: tert-butyl N-methyl-N-[[4-[2-[methyl-(2-methyl-4-nitro-benzoyl)amino]ethyl]morpholin-2-yl]methyl]carbamate

A mixture of N-(2-chloroethyl)-N,2-dimethyl-4-nitro-benzamide (230 mg, 0.900 mmol), tert-butyl N-methyl-N-(morpholin-2-ylmethyl)carbamate (250 mg, 1.09 mmol), N,N-diisopropylethylamine (0.62 mL, 3.58 mmol) in DMSO (5 mL) was stirred at 100° C. for 16 h. After cooling to room temperature, the mixture was diluted with ethyl acetate (100 mL), washed with water (30 mL), brine (30 mL×2), dried over sodium sulphate and concentrated under reduced pressure. The residue was purified by prep-HPLC to give tert-butyl N-methyl-N-[[4-[2-[methyl-(2-methyl-4-nitro-benzoyl)amino]ethyl]morpholin-2-yl]methyl]carbamate (160 mg) as a light-yellow oil.

### Step 2: tert-butyl N-[[4-[2-[(4-amino-2-methyl-benzoyl)-methyl-amino]ethyl]morpholin-2-yl]methyl]-N-methyl-carbamate

To a solution of N-methyl-N-[[4-[2-[methyl-(2-methyl-4-nitro-benzoyl)amino]ethyl]morpholin-2-yl]methyl]carbamate (160 mg, 0.360 mmol) in ethyl acetate (3 mL) was added Pd/C (10%, 20 mg). The mixture was hydrogenated at 20° C. under H₂ for 16 h. The mixture was filtered and the filtrate was concentrated under reduced pressure to give crude tert-butyl N-[[4-[2-[(4-amino-2-methyl-benzoyl)-methyl-amino]ethyl]morpholin-2-yl]methyl]-N-methyl-carbamate (120 mg) as a yellow solid.

### Step 3: tert-butyl N-[[4-[2-[[4-[[3-(3-fluoro-4-methoxy-phenyl)imidazo[1,2-a]pyrazin-8-yl]amino]-2-methyl-benzoyl]-methyl-amino]ethyl]morpholin-2-yl]methyl]-N-methyl-carbamate

A mixture of 8-chloro-3-(3-fluoro-4-methoxy-phenyl)imidazo[1,2-a]pyrazine (80 mg, 0.290 mmol), N-[[4-[2-[(4-amino-2-methyl-benzoyl)-methyl-amino]ethyl]morpholin-2-yl]methyl]-N-methyl-carbamate (120 mg, 0.290 mmol), Brettphos Pd G3 (27 mg, 0.030 mmol, cas #1470372-59-8), potassium carbonate (118 mg, 0.850 mmol) in t-BuOH (1.5 mL) was stirred at 100° C. for 16 h. The mixture was diluted with ethyl acetate (100 mL), washed with water (30 mL), brine (30 mL), dried over sodium sulphate and concentrated under reduced pressure. The residue was purified by prep-TLC (DCM/MeOH=15/1) to give tert-butyl N-[[4-[2-[[4-[[3-(3-fluoro-4-methoxy-phenyl)imidazo[1,2-a]pyrazin-8-yl]amino]-2-methyl-benzoyl]-methyl-amino]ethyl]morpholin-2-yl]methyl]-N-methyl-carbamate (120 mg) as a yellow oil.

### Step 4: 4-[[3-(3-fluoro-4-methoxy-phenyl)imidazo[1,2-a]pyrazin-8-yl]amino]-N,2-dimethyl-N-[2-[2-(methylaminomethyl)morpholin-4-yl]ethyl]benzamide

To a solution of tert-butyl N-[[4-[2-[[4-[[3-(3-fluoro-4-methoxy-phenyl)imidazo[1,2-a]pyrazin-8-yl]amino]-2-methyl-benzoyl]-methyl-amino]ethyl]morpholin-2-yl]methyl]-N-methyl-carbamate (120 mg, 0.180 mmol) in DCM (3 mL) was added HCl in dioxane (1.6 mL, 6.4 mmol). The mixture was stirred at 20° C. for 16 h. The mixture was concentrated under reduced pressure. The residue was purified by prep-HPLC to give the title compound (21 mg) as a white solid. MS obsd. (ESI⁺) [(M+H)⁺]: 562.1

### Reference Example 244

### N-(2-(2-(4-hydroxypiperidin-1-yl)ethoxy)ethyl)-4-((3-(4-methoxyphenyl)imidazo[1,2-a]pyrazin-8-yl)amino)-2-methylbenzamide

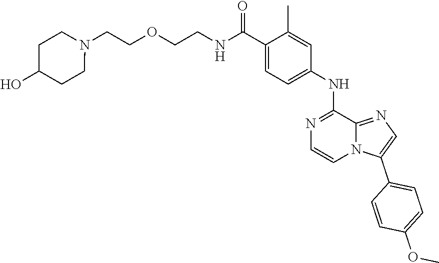 [SMILES: COc1ccc(-c2cnc3c(Nc4ccc(C(=O)NCCOCCN5CCC(O)CC5)c(C)c4)nccn23)cc1]

A mixture of N-(2-(2-chloroethoxy)ethyl)-4-((3-(4-methoxyphenyl)imidazo[1,2-a]pyrazin-8-yl)amino)-2-methylbenzamide (Reference Example 51, 30 mg, 62.5 μmol, Eq: 1), piperidin-4-ol (9.5 mg), sodium carbonate (9.94 mg, 93.8 μmol, Eq: 1.50) and potassium iodide (519 μg, 3.13 μmol, Eq: 0.05) in n-BuOH (0.5 mL) was heated at 105° C. for 48 h. Water was added to the reaction mixture and extracted with DCM. The combined organic layers were dried over sodium sulphate and concentrated to an oil. The product was purified by prep. HPLC to give the title compound (19 mg). MS (ESI, m/z): 547.4.

The following examples were prepared in analogy to Reference Example 244, Boc-protected intermediates were deprotected using HCl (4M in dioxane).

### Intermediate 89

### N-(2-(2-chloroethoxy)ethyl)-4-((3-(4-(difluoromethoxy)phenyl)imidazo[1,2-a]pyrazin-8-yl)amino)-2-methylbenzamide

The title compound was prepared in analogy to Reference Example 51 from Intermediate 7 and 2-(2-chloroethoxy)ethanamine hydrochloride. MS (ESI⁺) [(M+H)⁺]: 516.3

The following examples were prepared in analogy to Reference Example 244

| | | | MS | |
|---|---|---|---|---|
| | | | ESI | |
| Ex. | Name | Structure | [M + H]⁺ | Starting Material |
| REF 245 | 4-((3-(4- methoxyphenyl)imida- zo[1,2-a]pyrazin-8- yl)amino)-2-methyl- N-(2-(2- (methylamino)ethoxy) ethyl)benzamide | 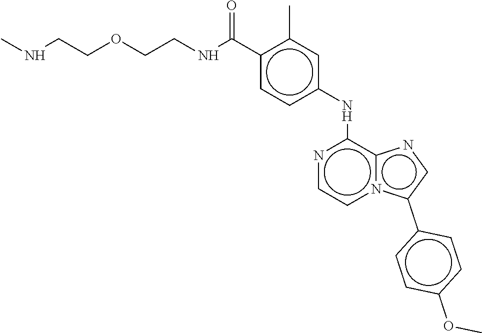 | 475.4 | Reference Example 51 and methanamine |
| REF 246 | N-(2-(2-((2- hydroxyethyl)amino) ethoxy)ethyl)-4-((3- (4- methoxyphenyl)imida- zo[1,2-a]pyrazin-8- yl)amino)-2- methylbenzamide | 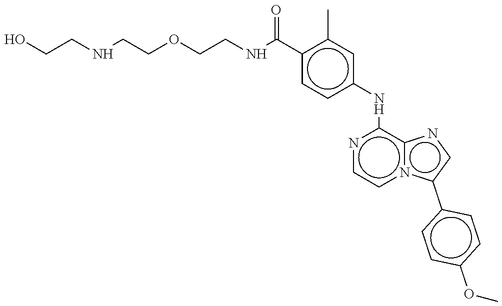 | 505.4 | Reference Example 51 and 2- aminoethanol |
| REF 247 | N-(2-(2-((2,2- difluoroethyl)amino) ethoxy)ethyl)-4-((3- (4- methoxyphenyl)imida- zo[1,2-a]pyrazin-8- yl)amino)-2- methylbenzamide | 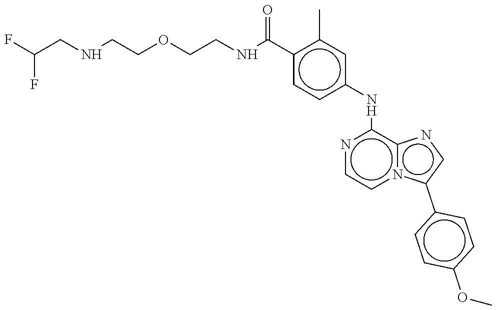 | 525.4 | Reference Example 51 and 2,2- difluoroethanamine |
| REF 248 | N-(2-(2-(2-oxa-6- azaspiro[3.3]heptan- 6-yl)ethoxy)ethyl)-4- ((3-(4- methoxyphenyl)imida- zo[1,2-a]pyrazin-8- yl)amino)-2- methylbenzamide | 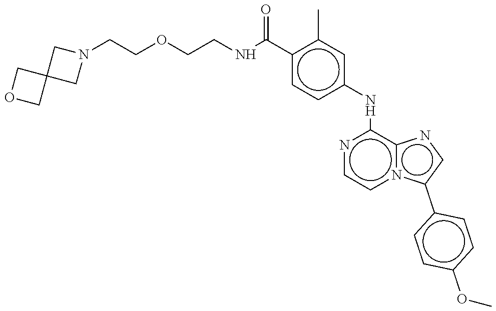 | 543.5 | Reference Example 51 and 2-oxa-6- azaspiro[3.3]heptane |
| REF 249 | N-(2-(2-(4- fluoropiperidin-1- yl)ethoxy)ethyl)-4- ((3-(4- methoxyphenyl)imida- zo[1,2-a]pyrazin-8- yl)amino)-2- methylbenzamide | 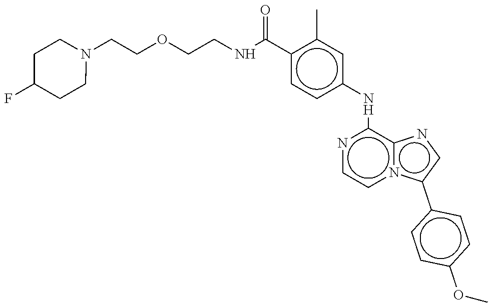 | 547.4 | Reference Example 51 and 4- fluoropiperidine |
| REF 250 | 4-[[3-(4- methoxyphenyl)imida- zo[1,2-a]pyrazin-8- yl]amino]-2-methyl- N-[2-(2-piperazin-1- ylethoxy)ethyl]benza- mide; dihydrochloride | 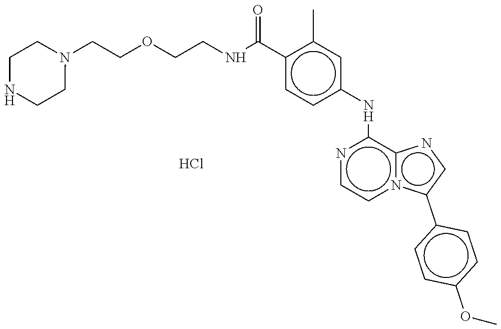 | 530.5 | Reference Example 51 and tert-butyl piperazine-1- carboxylate |
| REF 251 | 4-((3-(4- (difluoromethoxy)phe- nyl)imidazo[1,2- a]pyrazin-8- yl)amino)-2-methyl- N-(2-(2- (methylamino)ethoxy) ethyl)benzamide | 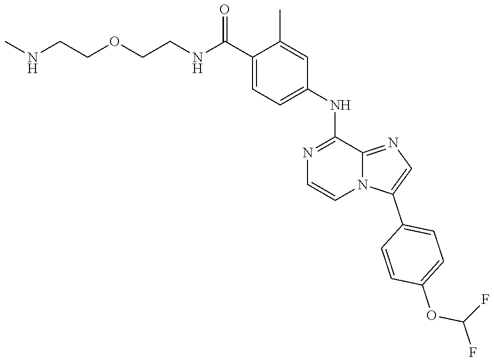 [SMILES: CNCCOCCNC(=O)c1ccc(Nc2nccn3c(-c4ccc(OC(F)F)cc4)cnc23)cc1C] | 511.4 | Intermediate 89 and methylamine |

### Intermediate 90

### 1-(4-amino-2-chloro-benzoyl)piperidine-4-carboxylic Acid

### Step 1

### Methyl 1-(4-amino-2-chloro-benzoyl)piperidine-4-carboxylate

To a solution of 4-amino-2-chlorobenzoic acid (1.70 g, 10 mmol), methyl piperidine-4-carboxylate (2.85 g, 20 mmol) in anhydrous DCM (50 mL) was added DIPEA (2.58 g, 20 mmol) and then the resultant mixture was stirred for 30 min at room temperature, HATU (7.6 g, 20 mmol) was added in the mixture and stirred for extra 10 h. The mixture was poured into water (100 mL) and the aqueous solution was extracted with DCM (100 mL×2). The organic layers were combined and washed with water and brine, dried over anhydrous sodium sulphate and concentrated under reduced pressure to give a red oil, which was purified by flash column chromatography to to provide the desired compound (1.82 g, 61.3% yield) as a white solid. MS (ESI, m/z): 297.1 [M+H]+.

### Step 2

### 1-(4-amino-2-chloro-benzoyl)piperidine-4-carboxylic Acid

To a solution of methyl 1-(4-amino-2-chlorobenzoyl)piperidine-4-carboxylate (890 mg, 3.0 mmol) in THF (5 mL) and methanol (25 mL) was added 2.0 M aq. LiOH (3.0 mL). The resultant mixture was stirred for 15 h at room temperature and then acidified to pH=5-6 with 3.0 M hydrochloric acid. The resulting suspension was filtered, the solid was washed with water and then dried to give the title compound (0.6 g, 70.7% yield) as a white solid. MS (ESI, m/z): 283.0 [M+H]+.

### Intermediate 91

### 1-(4-amino-2-methyl-benzoyl)piperidine-4-carboxylic Acid

### Step 1

### Methyl 1-(2-methyl-4-nitro-benzoyl)piperidine-4-carboxylate

To a solution of 2-methyl-4-nitrobenzoic acid (1.8 g, 10 mmol), methyl piperidine-4-carboxylate (2.85 g, 20 mmol) in anhydrous DCM (50 mL) was added DIPEA (2.58 g, 20 mmol) and then the resultant mixture was stirred for 30 min at room temperature, HATU (7.6 g, 20 mmol) was added in the mixture and stirred for extra 10 h. The mixture was poured into water (100 mL) and the aqueous solution was extracted with DCM (100 mL×2). The organic layers were combined and washed with water and brine, dried over anhydrous sodium sulphate and concentrated under reduced pressure to give a red oil, which was purified by flash column chromatography to provide the desired compound (2.86 g, 93.4% yield) as a yellow solid. MS (ESI, m/z): 307.1 [M+H]+.

### Step 2

### Methyl 1-(4-amino-2-methyl-benzoyl)piperidine-4-carboxylate

To a solution of methyl 1-(2-methyl-4-nitro-benzoyl)piperidine-4-carboxylate (3.0 g, 9.3 mmol) in EtOH (50 mL) was added palladium on carbon (254 mg, 0.1 mol). The mixture was degassed and charged with a H₂ balloon. The reaction was stirred at room temperature overnight. The catalyst was filtered off and the filtrate was concentrated. The residue was purified by column chromatography to give the final compound (1.93 g, 70% yield) as a red oil. MS (ESI, m/z): 277.1 [M+H]+.

### Step 3

### 1-(4-amino-2-methyl-benzoyl)piperidine-4-carboxylic Acid

To a solution of methyl 1-(4-amino-2-methylbenzoyl)piperidine-4-carboxylate (830 mg, 3.0 mmol) in THF (5 mL) and methanol (25 mL) was added 2.0 M aq LiOH (6.0 mL). The resultant mixture was stirred for 15 h at room temperature and then acidified to pH=5-6 with 3.0 M hydrochloric acid. The resulting suspension was filtered, the solid was washed with water and then dried to give the title compound (0.6 g, 76.2% yield) as a white solid. MS (ESI, m/z): 263.1 [M+H]+.

### Intermediate 92

### 1-[2-chloro-4-[[3-[4-(cyanomethoxy)-2,3-difluoro-phenyl]imidazo[1,2-a]pyrazin-8-yl]amino]benzoyl]piperidine-4-carboxylic Acid

To a solution of intermediate 30 (0.96 g, 3.0 mmol) in acetonitrile (30 mL) and acetic acid (3.0 mL) was added intermediate 90 (0.85 g, 3.0 mmol) and then stirred overnight at 95° C. The mixture was poured into water (50 mL) and the resulting suspension filtered. The solid was washed with acetonitrile and water, dried to give the title compound (1.0 g, 58.8% yield) as a light red solid which was used in next step without purification. MS (ESI, m/z): 567.1 [M+H]+.

The following intermediates were prepared in analogy to intermediate 92

| | | ESI MS | |
|---|---|---|---|
| Int. | Name | [M + H]⁺ | Starting Material |
| 93 | 1-[2-chloro-4-[[3-[4-(cyanomethoxy)-2,3- | 583.1 | Intermediate 90 and |
| | difluoro-phenyl]imidazo[1,2-a]pyrazin-8- | | intermediate 41 |
| | yl]amino]benzoyl]piperidine-4-carboxylic acid | | |
| 94 | 1-[4-[[3-[4-(cyanomethoxy)-2,3-difluoro- | 547.1 | Intermediate 91 |
| | phenyl]imidazo[1,2-a]pyrazin-8-yl]amino]-2- | | and intermediate 30 |
| | methyl-benzoyl]piperidine-4-carboxylic acid | | |
| 95 | 1-[4-[[3-[2-chloro-4-(cyanomethoxy)-3-fluoro- | 563.1 | Intermediate 90 and |
| | phenyl]imidazo[1,2-a]pyrazin-8-yl]amino]-2- | | intermediate 41 |
| | methyl-benzoyl]piperidine-4-carboxylic acid | | |

### Reference Example 252

### 2-[2,3-difluoro-4-[8-[4-[4-[3-(hydroxymethyl)piperazine-1-carbonyl]piperidine-1-carbonyl]-3-methyl-anilino]imidazo[1,2-a]pyrazin-3-yl]phenoxy]acetonitrile;2,2,2-trifluoroacetic Acid

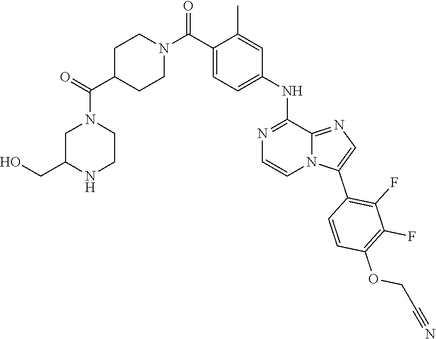 [SMILES: Cc1cc(Nc2nccn3c(-c4ccc(OCC#N)c(F)c4F)cnc23)ccc1C(=O)N1CCC(C(=O)N2CCNC(CO)C2)CC1]

### Step 1

### Tert-butyl 4-[1-[4-[[3-[4-(cyanomethoxy)-2,3-difluoro-phenyl]imidazo[1,2-a]pyrazin-8-yl]amino]-2-methyl-benzoyl]piperidine-4-carbonyl]-2-(hydroxymethyl)piperazine-1-carboxylate

To a solution of intermediate 94 (273 mg, 0.5 mmol), tert-butyl 2-(hydroxymethyl)piperazine-1-carboxylate (130 mg, 0.6 mmol) in anhydrous DMF (10 mL) was added DIPEA (258 mg, 2.0 mmol) and then the resultant mixture was stirred for 30 min at room temperature, T₃P (0.5 mL, 0.75 mmol) was added to the mixture and stirred for extra 10 h. The mixture was poured into water (50 mL) and the aqueous solution was extracted with DCM (50 mL×2). The organic layers were combined and washed with water and brine, dried over anhydrous sodium sulphate and concentrated under reduced pressure to give a red oil which was used in next step without purification. MS (ESI, m/z): 745.3 [M+H]⁺.

### Step 2

### 2-[2,3-difluoro-4-[8-[4-[4-[3-(hydroxymethyl)piperazine-1-carbonyl]piperidine-1-carbonyl]-3-methyl-anilino]imidazo[1,2-a]pyrazin-3-yl]phenoxy]acetonitrile;2,2,2-trifluoroacetic Acid

To a solution of tert-butyl 4-(1-(4-((3-(4-(cyanomethoxy)-2,3-difluorophenyl)imidazo[1,2-a]pyrazin-8-yl)amino)-2-methylbenzoyl)piperidine-4-carbonyl)-2-(hydroxymethyl)piperazine-1-carboxylate (300 mg, 0.4 mmol) in THF (5 mL) was added 3M hydrochloric acid (2.0 mL) at room temperature. The resultant mixture was stirred for 10 h and then adjusted to pH=7-8 with 2M Na₂CO₃ aqueous solution. The mixture was extracted with DCM (50 mL×2), the combined organic layers were washed with water and brine, dried over anhydrous sodium sulphate, and concentrated to give a red oil which was purified by prep. HPLC to provide the desired compound (215 mg, 79.2% yield) as an off-white powder. MS (ESI, m/z): 645.2 [M+H]+.

The following examples were prepared in analogy to Reference Example 252

| | | ESI MS | |
|---|---|---|---|
| Int. | Name | [M + H]⁺ | Starting Material |
| REF | piperazin-2-ylmethyl 1-[2-chloro-4-[[3-[2-chloro- | 681.1 | Intermediate 93 |
| 253 | 4-(cyanomethoxy)-3-fluoro-phenyl]imidazo[1,2- | | and tert-butyl 3- |
| | a]pyrazin-8-yl]amino]benzoyl]piperidine-4- | | (hydroxymethyl)piperazine- |
| | carboxylate trifluoroacetate | | 1-carboxylate |
| REF | piperazin-2-ylmethyl 1-[2-chloro-4-[[3-[4- | 665.2 | Intermediate 92 |
| 107 | (cyanomethoxy)-2,3-difluoro- | | and tert-butyl 3- |
| | phenyl]imidazo[1,2-a]pyrazin-8- | | (hydroxymethyl)piperazine- |
| | yl]amino]benzoyl]piperidine-4-carboxylate | | 1-carboxylate |
| | trifluoroacetate | | |
| 30 | 2-[4-[8-[3-chloro-4-[4-[2- | 665.2 | Intermediate 92 and |
| | (hydroxymethyl)piperazine-1- | | tert-butyl 3- |
| | carbonyl]piperidine-1- | | (hydroxymethyl)piperazine- |
| | carbonyl]anilino]imidazo[1,2-a]pyrazin-3-yl]-2,3- | | 1-carboxylate |
| | difluoro-phenoxy]acetonitrile trifluoroacetate | | |
| REF | 2-[3-chloro-2-fluoro-4-[8-[4-[4-[3- | 661.2 | Intermediate 95 and |
| 254 | (hydroxymethyl)piperazine-1- | | tert-butyl 2- |
| | carbonyl]piperidine-1-carbonyl]-3-methyl- | | (hydroxymethyl)piperazine- |
| | anilino]imidazo[1,2-a]pyrazin-3- | | 1-carboxylate |
| | yl]phenoxy]acetonitrile trifluoroacetate | | |
| REF | piperazin-2-ylmethyl 1-[2-chloro-4-[[3-[4- | 661.2 | Intermediate 95 and |
| 255 | (cyanomethoxy)-2,3-difluoro- | | tert-butyl 3- |
| | phenyl]imidazo[1,2-a]pyrazin-8- | | (hydroxymethyl)piperazine- |
| | yl]amino]benzoyl]piperidine-4-carboxylate | | 1-carboxylate |
| | trifluoroacetate | | |
| 31 | 2-[3-chloro-4-[8-[3-chloro-4-[4-[2- | 681.1 | Intermediate 93 |
| | (hydroxymethyl)piperazine-1- | | and tert-butyl 3- |
| | carbonyl]piperidine-1- | | (hydroxymethyl)piperazine- |
| | carbonyl]anilino]imidazo[1,2-a]pyrazin-3-yl]-2- | | 1-carboxylate |
| | fluoro-phenoxy]acetonitrile trifluoroacetate | | |
| REF | 2-[2,3-difluoro-4-[8-[4-[4-[3- | 645.2 | Intermediate 94 |
| 252 | (hydroxymethyl)piperazine-1- | | and tert-butyl 2- |
| | carbonyl]piperidine-1-carbonyl]-3-methyl- | | (hydroxymethyl)piperazine- |
| | anilino]imidazo[1,2-a]pyrazin-3- | | 1-carboxylate |
| | yl]phenoxy]acetonitrile trifluoroacetate | | |
| 32 | 1-[2-chloro-4-[[3-[4-(cyanomethoxy)-2,3- | 623.2 | Intermediate 92 |
| | difluoro-phenyl]imidazo[1,2-a]pyrazin-8- | | and tert-butyl (2- |
| | yl]amino]benzoyl]-N-[2- | | aminoethyl)(methyl)carbamate |
| | (methylamino)ethyl]piperidine-4-carboxamide | | |
| | trifluoroacetate | | |
| REF | 1-[4-[[3-[4-(cyanomethoxy)-2,3-difluoro- | 642.2 | Intermediate 94 |
| 256 | phenyl]imidazo[1,2-a]pyrazin-8-yl]amino]-2- | | and 2-(2H-tetrazol- |
| | methyl-benzoyl]-N-[2-(1H-tetrazol-5- | | 5-yl)ethan-1-amine |
| | yl)ethyl]piperidine-4-carboxamide | | hydrochloride |
| | trifluoroacetate | | |
| REF | 1-[4-[[3-[4-(cyanomethoxy)-2,3-difluoro- | 624.1 | Intermediate 94 |
| 257 | phenyl]imidazo[1,2-a]pyrazin-8-yl]amino]-2- | | and |
| | methyl-benzoyl]-N-methylsulfonyl-piperidine-4- | | methanesulfonamide |
| | carboxamide trifluoroacetate | | |
| REF | N-(2-amino-3 -hydroxypropyl)-1-(4-((3-(3-fluoro- | 576.3 | Reference Example |
| 258 | 4-methoxyphenyl)imidazo[1,2-a]pyrazin-8- | | 2 and tert-butyl (1- |
| | yl)amino)-2-methylbenzoyl)piperidine-4- | | amino-3- |
| | carboxamide hydrochloride | | hydroxypropan-2- |
| | | | yl)carbamate |

### Intermediate 96

### 2-[4-[8-[3-chloro-4-(piperazine-1-carbonyl)anilino]imidazo[1,2-a]pyrazin-3-yl]-2,3-difluoro-phenoxy]acetonitrile

### Step 1

### Tert-butyl 4-[1-[4-[[3-[4-(cyanomethoxy)-2,3-difluoro-phenyl]imidazo[1,2-a]pyrazin-8-yl]amino]-2-methyl-benzoyl]piperidine-4-carbonyl]-2-(hydroxymethyl)piperazine-1-carboxylate

To a solution of intermediate 29 (1.82 g, 4 mmol), tert-butyl piperazine-1-carboxylate (0.9 g, 4.8 mmol) in anhydrous DMF (35 mL) was added DTPEA (2.6 g, 20 mmol) and then the resultant mixture was stirred for 30 min at room temperature, T₃P (4 mL, 6.4 mmol) was added in the mixture and stirred for extra 10 h. The mixture was poured into water (50 mL) and the aqueous solution was extracted with DCM (50 mL×2). The organic layers were combined and washed with water and brine, dried over anhydrous sodium sulphate and concentrated under reduced pressure to give a red oil which was used in next step without purification. MS (ESI, m/z): 624.1 [M+H]+.

### Step 2

### 2-[4-[8-[3-chloro-4-(piperazine-1-carbonyl)anilino]imidazo[1,2-a]pyrazin-3-yl]-2,3-difluoro-phenoxy]acetonitrile

To a solution of tert-butyl 4-[1-[4-[[3-[4-(cyanomethoxy)-2,3-difluoro-phenyl]imidazo[1,2-a]pyrazin-8-yl]amino]-2-methyl-benzoyl]piperidine-4-carbonyl]-2-(hydroxymethyl)piperazine-1-carboxylate (1.8 g, 3 mmol) in THF (15 mL) was added 3M hydrochloric acid aqueous solution (10 mL) at room temperature. The resultant mixture was stirred for 10 h and then adjusted to pH=7-8 with ammonia solution. The mixture was poured into water (25 mL) and then extracted with dichloromethane/isopropanol (100/10 mL), the organic layer was concentrated to give a red oil, which was purified by prep. HPLC to provide the title compound (1.2 g, 79.4% yield) as a light red solid. MS (ESI, m/z): 524.1 [M+H]+.

The following intermediates were prepared in analogy to intermediate 96

| | | ESI MS | |
|---|---|---|---|
| Int. | Name | [M + H]⁺ | Starting Material |
| 97 | 2-[3-chloro-4-[8-[3-chloro-4-(piperazine-1- | 539.1 | Intermediate 24 and |
| | carbonyl)anilino]imidazo[1,2-a]pyrazin-3-yl]-2- | | tert-butyl |
| | fluoro-phenoxy] acetonitrile | | piperazine-1- |
| | | | carboxylate |
| 98 | [2-chloro-4-[[3-(2,3-difluoro-4-methoxy- | 499.1 | Intermediate 20 and |
| | phenyl)imidazo[1,2-a]pyrazin-8-yl]amino]phenyl]- | | tert-butyl |
| | piperazin-1-yl-methanone | | piperazine-1- |
| | | | carboxylate |
| 76 | (4-((3-iodoimidazo[1,2-a]pyrazin-8-yl)amino)-2- | 463.3 | Intermediate 1 |
| | methylphenyl)(piperazin-1-yl)methanone | | and tert- |
| | hydrochloride | | butylpiperazine-1- |
| | | | carboxylate |

### Example 33

### 2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile Formate

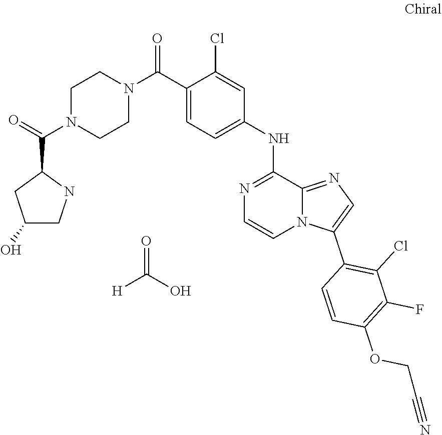 [SMILES: N#CCOc1ccc(-c2cnc3c(Nc4ccc(C(=O)N5CCN(C(=O)[C@@H]6C[C@@H](O)CN6)CC5)c(Cl)c4)nccn23)c(Cl)c1F ; O=CO]

### Step 1

### tert-butyl (2S,4R)-2-[4-[2-chloro-4-[[3-[2-chloro-4-(cyanomethoxy)-3-fluoro-phenyl]imidazo[1,2-a]pyrazin-8-yl]amino]benzoyl]piperazine-1-carbonyl]-4-hydroxy-pyrrolidine-1-carboxylate

To a solution of intermediate 97 (162 mg, 0.3 mmol), (2S,4R)-1-(tert-butoxycarbonyl)-4-hydroxypyrrolidine-2-carboxylic acid (83 mg, 0.36 mmol) was added DIPEA (78 mg, 0.6 mmol), the resultant mixture was stirred for 10 min at room temperature, and then HATU (228 mg, 0.6 mmol) was added in the mixture and stirred for extra 10 h at room temperature. The mixture was poured into water (50 mL) and the aqueous solution was extracted with DCM (50 mL×2). The organic layers were combined and washed with water and brine, dried over anhydrous sodium sulphate and concentrated under reduced pressure to give a red oil which was used in next step without purification. MS (ESI, m/z): 753.2 [M+H]+.

### Step 2

### 2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile Formate

To a solution of tert-butyl (2S,4R)-2-(4-(2-chloro-4-((3-(2-chloro-4-(cyanomethoxy)-3-fluorophenyl)imidazo[1,2-a]pyrazin-8-yl)amino)benzoyl)piperazine-1-carbonyl)-4-hydroxypyrrolidine-1-carboxylate (200 mg, 0.265 mmol) in THF (5 mL) was added 3M hydrochloric acid aqueous solution (1 mL) at room temperature. The resultant mixture was stirred for 10 h and then adjusted to pH=7-8 with ammonia solution. The mixture was poured into water (25 mL) and then extracted with dichloromethane/isopropanol (50/5 mL), the organic layer was concentrated to give a red oil, which was purified by prep. HPLC to provide the title compound (20 mg, 11.3% yield) as a white powder. MS (ESI, m/z): 653.2 [M+H]+.

The following examples were prepared in analogy to example 33

| | | | ESI MS | |
|---|---|---|---|---|
| Ex. | Name | Structure | [M + H]+ | Starting Material |
| 34 | 2-[4-[8-[3-chloro-4-[4- [(2S,4R)-4- hydroxypyrrolidine-2- carbonyl]piperazine-1- carbonyl]anilino]imidazo [1,2-a]pyrazin-3-yl]- 2,3-difluroo- phenoxy]acetonitrile formate | 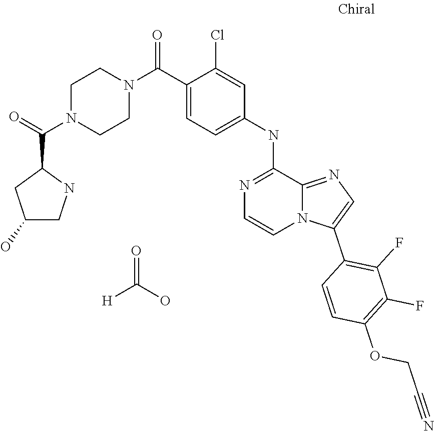 | 637.1 | Intermediate 96 and (2S,4R)-1-(tert- butoxycarbonyl)-4- hydroxypyrrolidine- 2-carboxylic acid |
| 35 | [2-chloro-4-[[3-(2,3- difluoro-4-methoxy- phenyl)imidazo[1,2- a]pyrazin-8- yl]amino]phenyl]-[4- [(2S,4R)-4- hydroxypyrrolidine-2- carbonyl]piperazin-1- yl]methanone formate | 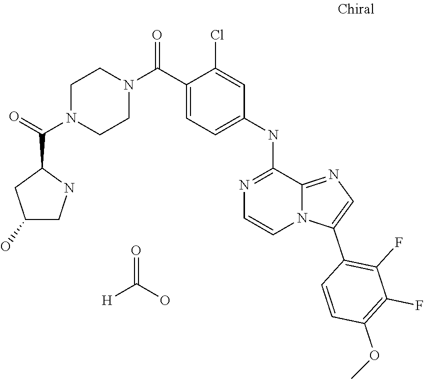 | 612.1 | Intermediate 98 and (2S,4R)-1-(tert- butoxycarbonyl)-4- hydroxypyrrolidine- 2-carboxylic acid |

### Example 36

### 2-[4-[8-[3-chloro-4-[4-[(3R)-3-(hydroxymethyl)piperazine-1-carbonyl]piperazine-1-carbonyl]anilino]imidazo[1,2-a]pyrazin-3-yl]-2,3-difluoro-phenoxy]acetonitrile

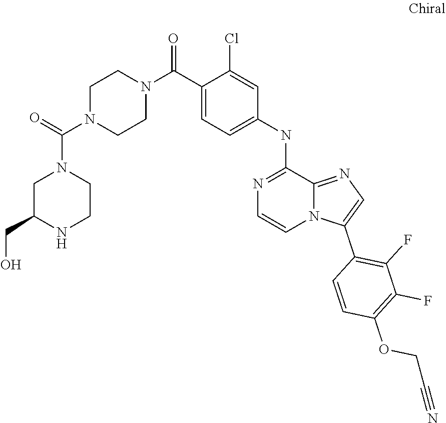 [SMILES: N#CCOc1ccc(-c2cnc3c(Nc4ccc(C(=O)N5CCN(C(=O)N6CCN[C@@H](CO)C6)CC5)c(Cl)c4)nccn23)c(F)c1F]

### Step 1

### Tert-butyl (2R)-4-[4-[2-chloro-4-[[3-[4-(cyanomethoxy)-2,3-difluoro-phenyl]imidazo[1,2-a]pyrazin-8-yl]amino]benzoyl]piperazine-1-carbonyl]-2-(hydroxymethyl)piperazine-1-carboxylate

To a solution of intermediate 96 (210 mg, 0.4 mmol), DIPEA (258 mg, 2.0 mmol) in anhydrous DCM (10 mL) was added triphosgene (104 mg, 0.2 mmol) and then the resultant mixture was stirred for 1.0 h at 0° C., and then treated with tert-butyl (R)-2-(hydroxymethyl)piperazine-1-carboxylate (104 mg, 0.48 mmol), the reaction mixture was allowed to warm to room temperature. The mixture was poured into saturated aq. sodium bicarbonate (50 mL) and the aqueous solution was extracted with DCM (50 mL×2). The organic layers were combined and washed with water and brine, dried over anhydrous sodium sulphate and concentrated under reduced pressure to give a red oil which was used in next step without purification. MS (ESI, m/z): 766.2 [M+H]+.

### Step 2

### 2-[4-[8-[3-chloro-4-[4-[(3R)-3-(hydroxymethyl)piperazine-1-carbonyl]piperazine-1-carbonyl]anilino]imidazo[1,2-a]pyrazin-3-yl]-2,3-difluoro-phenoxy]acetonitrile

To a solution of tert-butyl (2R)-4-[4-[2-chloro-4-[[3-[4-(cyanomethoxy)-2,3-difluoro-phenyl]imidazo[1,2-a]pyrazin-8-yl]amino]benzoyl]piperazine-1-carbonyl]-2-(hydroxymethyl)piperazine-1-carboxylate (153 mg, 0.2 mmol) in THF (10 mL) was added 3M hydrochloric acid (2 mL) at room temperature. The resultant mixture was stirred for 10 h and then adjusted to pH=7-8 with ammonia solution. The mixture was poured into water (30 mL) and then extracted with dichloromethane/isopropanol (100/10 mL), the organic layer was concentrated to give a red oil, which was purified by prep. HPLC to provide the title compound (18 mg, 13.3% yield) as a white powder. MS (ESI, m/z): 666.2 [M+H]+.

The following example was prepared in analogy to example 36

| | | | ESI MS | |
|---|---|---|---|---|
| Ex. | Name | Structure | [M + H]+ | Starting Material |
| 37 | 2-[4-[8-[3-chloro-4-[4- [(3S)-4- (hydroxymethyl)pipera- zine-1- carbonyl]piperazine-1- carbonyl]anilino]imida- zo[1,2-a]pyrazin-3-yl]- 2,3-difluoro- phenoxy]acetonitrile formate | 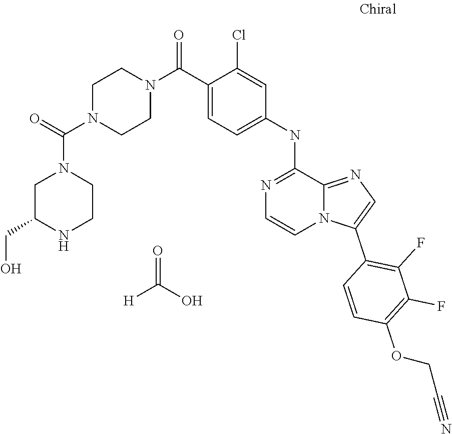 | 666.2 | Intermediate 96 and tert-butyl (S)-2- (hydroxymethyl)piper- azine-1- carboxylate |

### Reference Example 259

### N-[[1-[4-[[3-[4-(cyanomethoxy)-2,3-difluoro-phenyl]imidazo[1,2-a]pyrazin-8-yl]amino]-2-methyl-benzoyl]-4-piperidyl]methyl]-2-(methylamino)acetamide

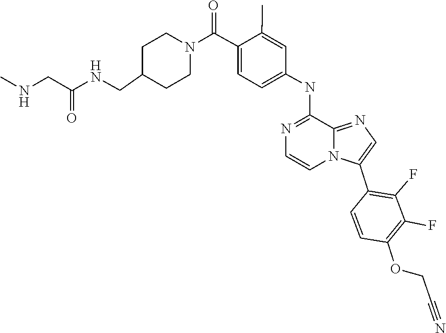

### Step 1

### tert-butyl N-[2-[[1-[4-[[3-[4-(cyanomethoxy)-2,3-difluoro-phenyl]imidazo[1,2-a]pyrazin-8-yl]amino]-2-methyl-benzoyl]-4-piperidyl]methylamino]-2-oxo-ethyl]-N-methyl-carbamate

To a solution of intermediate REF 129 (106 mg, 0.2 mmol), N-(tert-butoxycarbonyl)-N-methylglycine (57 mg, 0.3 mmol) in anhydrous DMF (10 mL) was added DIPEA (52 mg, 0.4 mmol) and then the resultant mixture was stirred for 30 min at room temperature, HATU (152 mg, 0.4 mmol) was added in the mixture and stirred for extra 10 h. The mixture was poured into water (50 mL) and the aqueous solution was extracted with DCM (50 mL×2). The organic layers were combined and washed with water and brine, dried over anhydrous sodium sulphate and concentrated under reduced pressure to give a red oil, which was used in next step without purification. MS (ESI, m/z): 703.2 [M+H]+.

### Step 2

### N-[[1-[4-[[3-[4-(cyanomethoxy)-2,3-difluoro-phenyl]imidazo[1,2-a]pyrazin-8-yl]amino]-2-methyl-benzoyl]-4-piperidyl]methyl]-2-(methylamino)acetamide

To a solution of tert-butyl (2-(((1-(4-((3-(4-(cyanomethoxy)-2,3-difluorophenyl)imidazo[1,2-a]pyrazin-8-yl)amino)-2-methylbenzoyl)piperidin-4-yl)methyl)amino)-2-oxoethyl)(methyl)carbamate (140 mg, 0.2 mmol) in THF (5 mL) was added 3M aq. hydrochloric acid (2.0 mL) at room temperature. The resultant mixture was stirred for 10 h and then adjusted to pH=7-8 with aq. ammonia. The mixture was poured into water (25 mL) and then extracted with dichloromethane/isopropanol (50/5 mL), the organic layer was concentrated to give a red oil, which was purified by prep. HPLC to provide the title compound (25 mg, 20.3% yield) as a white powder. MS (ESI, m/z): 603.2 [M+H]⁺.

### Reference Example 260

### N-((1-(4-((3-(4-methoxyphenyl)imidazo[1,2-a]pyrazin-8-yl)amino)-2-methylbenzoyl)piperidin-4-yl)methyl)acetamide

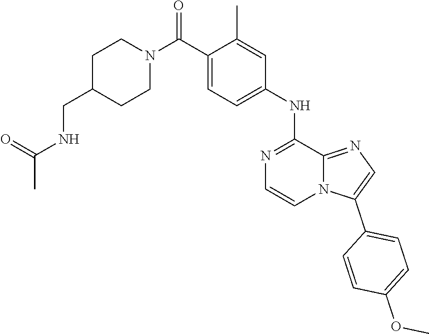 [SMILES: COc1ccc(-c2cnc3c(Nc4ccc(C(=O)N5CCC(CNC(C)=O)CC5)c(C)c4)nccn23)cc1]

tert-Butyl ((1-(4-((3-(4-methoxyphenyl)imidazo[1,2-a]pyrazin-8-yl)amino)-2-methylbenzoyl)piperidin-4-yl)methyl)carbamate (Intermediate obtained in the preparation of Reference Example 53, 200 mg) was treated with 1.05 eq acetyl chloride (0.032 mL) in 5 mL AcOEt/EtOH (9/1) the mixture was stirred overnight at room temperature. A mixture of Reference Example 53 and the title compound was obtained, which was separated by prep. HPLC. White powder (44 mg), MS (ESI, m/z): 513.4.

### Example 38

### (4-((3-(3-chloro-4-methoxyphenyl)imidazo[1,2-a]pyrazin-8-yl)amino)phenyl)(morpholino)methanone

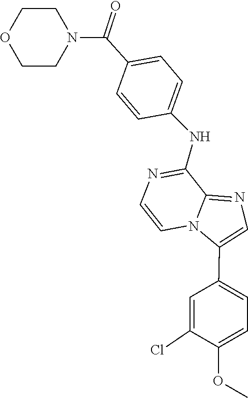 [SMILES: COc1ccc(-c2cnc3c(Nc4ccc(C(=O)N5CCOCC5)cc4)nccn23)cc1Cl]

A mixture of (4-aminophenyl)(morpholino)methanone (31 mg), Intermediate 15 (29.4 mg), potassium carbonate (27.6 mg), t-Bu-X-phos (2 mg) and Pd₂(dba)₃ (1 mg) in dioxane was stirred at 100° C. overnight. DMSO was added, the mixture was filtered over Celite and purified by prep. HPLC to give the title compound (9 mg) as a colorless solid.

MS (ESI, m/z): 464.2

The following examples were prepared in analogy:

| | | | MS | |
|---|---|---|---|---|
| | | | ESI | |
| Ex. | Name | Structure | [M + H]⁺ | Starting material |
| REF 261 | 2-chloro-4-((3-(4- (difluoromethoxy)phe- nyl)imidazo[1,2- a]pyrazin-8- yl)amino)-N- methylbenzamide | 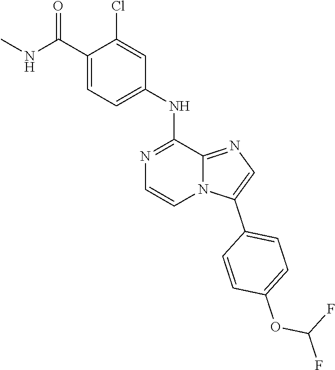 [SMILES: CNC(=O)c1ccc(Nc2nccn3c(-c4ccc(OC(F)F)cc4)cnc23)cc1Cl] | 444.3 | Intermediate 8 and 4- amino-2-chloro-N- methylbenzamide |
| REF 262 | 2-chloro-4-((3-(4- (difluoromethoxy)phe- nyl)imidazo[1,2- a]pyrazin-8- yl)amino)-N- (pyridin-2- ylmethyl)benzamide | 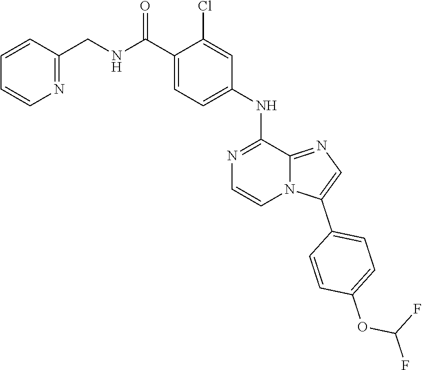 [SMILES: O=C(NCc1ccccn1)c1ccc(Nc2nccn3c(-c4ccc(OC(F)F)cc4)cnc23)cc1Cl] | 521.2 | Intermediate 8 and 4- amino-2-chloro-N- (pyridin-2- ylmethyl)benzamide |

### Reference Example 263

### [4-(aminomethyl)-1-piperidyl]-[4-[[3-(2-chloro-3-fluoro-4-methoxy-phenyl)imidazo[1,2-a]pyrazin-8-yl]amino]-2-methyl-phenyl]methanone Hydrochloride

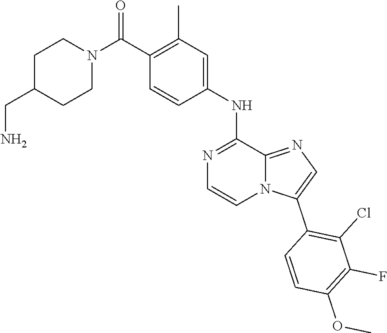 [SMILES: COc1ccc(-c2cnc3c(Nc4ccc(C(=O)N5CCC(CN)CC5)c(C)c4)nccn23)c(Cl)c1F]

### Step 1: tert-butyl N-[[1-(2-methyl-4-nitro-benzoyl)-4-piperidyl]methyl]carbamate

A mixture of 2-methyl-4-nitro-benzoic acid (1.00 g, 5.52 mmol), HATU (2.52 g, 6.62 mmol) and DIPEA (2.88 mL, 16.56 mmol) in DMF (25 mL) was stirred at 15° C. for 0.5 h. Then 4-(tert-butoxycarbonylaminomethyl) piperidine (1.42 g, 6.62 mmol) was added and the reaction was stirred at 15° C. for 16 h. The reaction mixture was diluted with H₂O (50 mL) and extracted with ethyl acetate. The organic phase was washed with brine, dried over anhydrous sodium sulphate, concentrated under reduced pressure and purified by flash column chromatography (eluting with DCM/MeOH=50/1) to afford desired compound (2.00 g) as a light yellow solid. MS obsd. (ESI⁺) [(M+Na)⁺]: 400

### Step 2: tert-butyl N-[[l-(4-amino-2-methyl-benzoyl)-4-piperidyl]methyl]carbamate

A mixture of tert-butyl N-[[1-(2-methyl-4-nitro-benzoyl)-4-piperidyl]methyl]carbamate (1.00 g, 2.65 mmol) and palladium on charcoal (100 mg, 10 wt. %) in methanol (10 mL) was stirred at 15° C. under H₂ for 16 h. The catalyst was filtered off and the filtrate was concentrated under reduced pressure to give desired compound (900 mg) as a red oil which was used directly for the next step.

### Step 3: tert-butyl N-[[l-[4-[[3-(2-chloro-3-fluoro-4-methoxy-phenyl)imidazo[1,2-a]pyrazin-8-yl]amino]-2-methyl-benzoyl]-4-piperidyl]methyl]carbamate

A mixture of intermediate 70 (50 mg, 0.16 mmol) and tert-butyl N-[[1-(4-amino-2-methyl-benzoyl)-4-piperidyl]methyl]carbamate (100 mg, 0.29 mmol) in acetonitrile (0.9 mL) and acetic acid (0.1 mL) was stirred at 90° C. for 16 h. The mixture was cooled to RT and concentrated under reduced pressure. The residue was purified by prep-TLC (DCM/MeOH=20/1) to afford desired compound (20 mg) as a white oil. MS obsd. (ESI⁺) [(M+H)⁺]: 623.1

### Step 4: [4-(aminomethyl)-1-piperidyl]-[4-[[3-(2-chloro-3-fluoro-4-methoxy-phenyl)imidazo[1,2-a]pyrazin-8-yl]amino]-2-methyl-phenyl]methanone Hydrochloride

To a stirred solution of tert-butyl N-[[1-[4-[[3-(2-chloro-3-fluoro-4-methoxy-phenyl)imidazo[1,2-a]pyrazin-8-yl]amino]-2-methyl-benzoyl]-4-piperidyl]methyl]carbamate (20 mg, 0.03 mmol) in methanol (0.5 mL) was added a solution of HCl in dioxane (0.04 mL 4.0 M) drop wise. The reaction mixture was stirred at 15° C. for 2 h. The reaction mixture was concentrated under reduced pressure and the residue was purified by prep. HPLC to give the title compound (5.8 mg) as a white solid. MS obsd. (ESI⁺) [(M+H)⁺]: 523.2.

### Reference Example 264

### 4-[[3-(3-fluoro-4-methoxy-phenyl)imidazo[1,2-a]pyrazin-8-yl]amino]-N-[2-(1H-imidazol-2-yl)ethyl]-N,2-dimethyl-benzamide

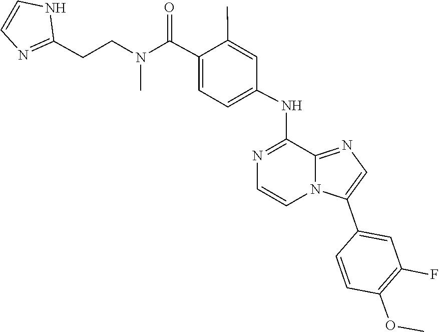 [SMILES: COc1ccc(-c2cnc3c(Nc4ccc(C(=O)N(C)CCc5ncc[nH]5)c(C)c4)nccn23)cc1F]

### Step 1: N-(3-hydroxypropyl)-N,2-dimethyl-4-nitro-benzamide

To a solution of 2-methyl-4-nitro-benzoic acid (1.0 g, 5.52 mmol) in DMF (10 mL) was added HATU (2.73 g, 7.18 mmol), 3-(methylamino)propan-1-ol (590 mg, 6.62 mmol) and triethylamine (1.68 g, 16.6 mmol). The mixture was stirred at 25° C. for 12 h and then diluted with ethyl acetate. The resulting mixture was washed with water and brine successively, dried over anhydrous sodium sulphate, concentrated under reduced pressure. The residue was purified by flash column chromatography (eluted with DCM/MeOH=20) to give the title compound (1.2 g) as a light yellow oil.

### Step 2: N,2-dimethyl-4-nitro-N-(3-oxopropyl)benzamide

To a stirred solution of N-(3-hydroxypropyl)-N,2-dimethyl-4-nitro-benzamide (900 mg, 3.57 mmol) and TEMPO (56 mg, 0.36 mmol) in DCM (10 mL) was added PhI(OAc)₂ (1.38 g, 4.28 mmol) slowly. The reaction was stirred at 20° C. for 1 h and then quenched with sat. Na₂SO₃ solution. The resulting mixture was extracted with DCM. The DCM layer was washed with brine, dried over anhydrous sodium sulphate, concentrated under reduced pressure and purified by flash column chromatography to afford the title compound (460 mg) as a light yellow oil.

### Step 3: N-[2-(1H-imidazol-2-yl)ethyl]-N,2-dimethyl-4-nitro-benzamide

To a stirred solution of N,2-dimethyl-4-nitro-N-(3-oxopropyl)benzamide (450 mg, 1.8 mmol) and ammonium hydroxide (1.76 g, 12.6 mmol) in methanol (5 mL) was added glyoxal (230 mg, 3.96 mmol) slowly. The reaction was stirred at 20° C. for 12 h. The mixture was diluted with H₂O (20 mL) and extracted with DCM (30 mL). The organic phase was washed with brine, dried over anhydrous sodium sulphate, concentrated under reduced pressure to afford the title compound (450 mg) as a light yellow oil.

### Step 4: 4-amino-N-[2-(1H-imidazol-2-yl)ethyl]-N,2-dimethyl-benzamide

Into a stirred solution of N-[2-(1H-imidazol-2-yl)ethyl]-N,2-dimethyl-4-nitro-benzamide (200 mg, 0.69 mmol) in methanol (5 mL) was added Pd on charcoal (74 mg, 10 wt. %). The reaction was stirred under H₂ balloon at 20° C. for 1 h. The catalyst was filtered off and the filtrate was concentrated under reduced pressure to afford the title compound (60 mg) as a light yellow oil which was used directly in next step.

### Step 5: 4-[[3-(3-fluoro-4-methoxy-phenyl)imidazo[1,2-a]pyrazin-8-yl]amino]-N-[2-(1H-imidazol-2-yl)ethyl]-N,2-dimethyl-benzamide

To a solution of Intermediate 3 (60 mg, 0.22 mmol) in tert-butanol (2 mL) was added BrettPhos-Pd-G3 (196 mg, 0.22 mmol, CAS #1470372-59-8), 4-amino-N-[2-(1H-imidazol-2-yl)ethyl]-N,2-dimethyl-benzamide (59 mg, 0.23 mmol), and potassium carbonate (30 mg, 0.22 mmol). The mixture was stirred at 100° C. for 12 h under N₂. After cooled to RT, the reaction mixture was diluted with ethyl acetate (100 mL). The resulting mixture was washed with water and brine successively, dried over anhydrous Na₂SO₄, concentrated under reduced pressure to afford crude product as a yellow oil. It was purified by prep-HPLC to give the title compound (37 mg) as a white solid. MS obsd. (ESI⁺) [(M+H)⁺]: 500.1

### Reference Example 265

### N-[2-[4-(2,2-difluoroethyl)piperazin-1-yl]ethyl]-4-[[3-(3-fluoro-4-methoxy-phenyl)imidazo[1,2-a]pyrazin-8-yl]amino]-N,2-dimethyl-benzamide; Formic Acid

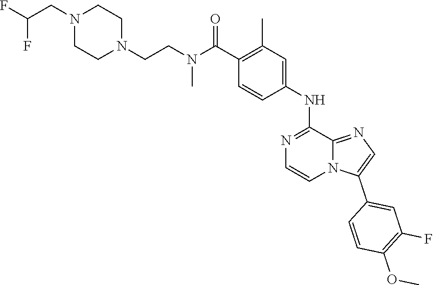 [SMILES: COc1ccc(-c2cnc3c(Nc4ccc(C(=O)N(C)CCN5CCN(CC(F)F)CC5)c(C)c4)nccn23)cc1F]

To a solution of REF 266 (50 mg, 0.09 mmol) in DMF (1 mL) was added potassium carbonate (37 mg, 0.27 mmol) and 1,1-difluoro-2-iodoethane (21 mg, 0.11 mmol). The reaction was stirred at 50° C. for 12 h and then was diluted with ethyl acetate. The resulting mixture was washed with water and brine successively, dried over anhydrous Na₂SO₄ and concentrated under reduced pressure to afford crude product as a yellow oil. It was purified by prep-HPLC to give the title compound (11 mg) as a white solid. MS obsd. (ESI⁺) [(M+H)⁺]: 582.

### Reference Example 267

### N-[2-(1-diethoxyphosphoryl-4-piperidyl)ethyl]-4-[[3-[4-(difluoromethoxy)phenyl]imidazo[1,2-a]pyrazin-8-yl]amino]-N,2-dimethyl-benzamide

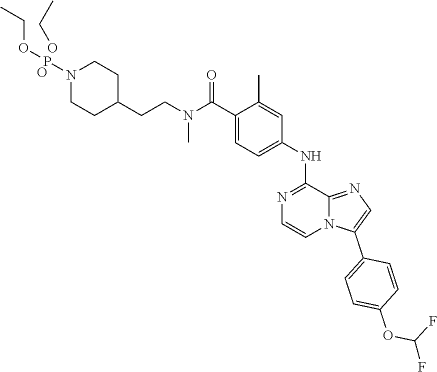

Into a stirred solution of Reference Example 174 (200 mg, 0.35 mmol) and triethylamine (0.15 mL, 1.05 mmol) in DCM (4 mL) was added diethyl chlorophosphate (200 mg, 1.16 mmol) slowly. The reaction was stirred at 15° C. for 2 h. The reaction was quenched with H₂O (5 mL) and extracted with DCM (10 mL×3). The organic phase was washed with brine (10 mL), dried over anhydrous Na₂SO₄, concentrated under reduced pressure and purified by prep-HPLC to afford the title compound (81.4 mg) as a white solid. MS obsd. (ESI⁺) [(M+23)⁺]: 671.1.

### Reference Example 268

### 2-[2-(dimethylamino)ethyl]-6-[[3-(3-fluoro-4-methoxy-phenyl)imidazo[1,2-a]pyrazin-8-yl]amino]-3,4-dihydroisoquinolin-1-one

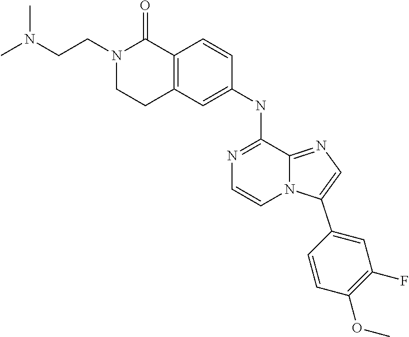 [SMILES: COc1ccc(-c2cnc3c(Nc4ccc5c(c4)CCN(CCN(C)C)C5=O)nccn23)cc1F]

### Step 1: 6-bromo-2-[2-(dimethylamino)ethyl]-3,4-dihydroisoquinolin-1-one

Into a stirred solution of 6-bromo-3, 4-dihydro-2H-isoquinolin-1-one (200 mg, 0.88 mmol) in DMF (5 mL) was added sodium hydride (53 mg, 1.33 mmol, 60 wt %) portion wise at 15° C. The mixture was stirred for 0.5 h. Then (2-bromoethyl)dimethylamine hydrobromide (309 mg, 1.33 mmol) was added and the reaction was stirred at 15° C. for 16 h. Sat. aq. NH₄Cl was added to quench the reaction. The obtained mixture was diluted with H₂O (10 mL) and extracted with ethyl acetate. The organic phase was washed with brine, dried over anhydrous Na₂SO₄, concentrated under reduced pressure and purified by prep-TLC (DCM/MeOH=20, Rf=0.1) to give the title compound (120 mg) as a light yellow oil.

### Step 2: 3-(3-fluoro-4-methoxy-phenyl)imidazo[1,2-a]pyrazin-8-amine

A mixture of 8-chloro-3-iodo-imidazo[1,2-a]pyrazine (500 mg, 1.8 mmol) and a solution of ammonium hydroxide (10 mL, 17.83 mmol) in 1,4-dioxane (5 mL) was stirred at 100° C. for 16 h in a sealed vessel. The reaction mixture was cooled and concentrated under reduced pressure. The residue was diluted with H₂O (20 mL) and extracted with DCM. The organic phase was dried over anhydrous Na₂SO₄ and concentrated under reduced pressure to give 3-(3-fluoro-4-methoxy-phenyl)imidazo[1,2-a]pyrazin-8-amine (300 mg) as a brown solid.

### Step 3: 2-[2-(dimethylamino)ethyl]-6-[[3-(3-fluoro-4-methoxy-phenyl)imidazo[1,2-a]pyrazin-8-yl]amino]-3,4-dihydroisoquinolin-1-one

A mixture of 3-(3-fluoro-4-methoxy-phenyl)imidazo[1,2-a]pyrazin-8-amine (100 mg, 0.39 mmol), 6-bromo-2-[2-(dimethylamino)ethyl]-3,4-dihydroisoquinolin-1-one (115 mg, 0.39 mmol), cesium carbonate (378 mg, 1.16 mmol), (R)-BINAP (48 mg, 0.08 mmol) and Pd₂(dba)₃ (22 mg, 0.04 mmol) in 1,4-dioxane (3 mL) was stirred under nitrogen at 100° C. for 16 h. The reaction mixture was cooled and filtered. The filtrate was concentrated under reduced pressure and purified by prep-TLC (DCM/MeOH=20, R_{f}=0.2) to get a crude product. It was re-purified by trituration (CH₃OH, 2 mL) to afford the title compound (17.9 mg) as a white solid. MS obsd. (ESI⁺) [(M+H)⁺]: 475.1.

### Reference Example 269

### 7-[[3-(3-fluoro-4-methoxy-phenyl)imidazo[1,2-a]pyrazin-8-yl]amino]-2,3,4,5-tetrahydro-2-benzazepin-1-one Hydrochloride

### Step 1: 6-aminotetralin-1-one Oxime

A mixture of 6-amino-1,2,3,4-tetrahydronaphthalen-1-one (1.0 g, 6.2 mmol), hydroxylamine hydrochloride (474 mg, 6.82 mmol), sodium acetate (1.12 g, 13.65 mmol) in ethanol (10 mL) and water (3.3 mL) was stirred at 90° C. for 4 h. The mixture was cooled to RT and diluted with H₂O (20 mL). The precipitate was collected by filtration and washed with water and dried over high vacuum to give 6-aminotetralin-1-one oxime (880 mg) as a white solid.

### Step 2: 7-amino-2,3,4,5-tetrahydro-2-benzazepin-1-one

A mixture of 6-aminotetralin-1-one oxime (880 mg, 4.99 mmol) in PPA (10 mL) was stirred at 120° C. for 2 h. The mixture was cooled to 90° C. and then poured onto ice. The resulting mixture was neutralized with 4N aq. NaOH and extracted with ethyl acetate. The ethyl acetate layer was washed with brine, dried over Na₂SO₄ and concentrated under reduced pressure to give crude compound (850 mg) (mixed of another isomer) as brown solid.

### Step 3: 7-[[3-(3-fluoro-4-methoxy-phenyl)imidazo[1,2-a]pyrazin-8-yl]amino]-2,3,4,5-tetrahydro-2-benzazepin-1-one Hydrochloride

A mixture of Intermediate 3 (150 mg, 0.540 mmol), crude 7-amino-2,3,4,5-tetrahydro-2-benzazepin-1-one (105 mg, 0.600 mmol) in acetonitrile (1.8 mL) and acetic acid (0.200 mL) was stirred at 90° C. for 16 h. The mixture was concentrated under reduced pressure and the residue was purified by prep. HPLC to give 7-[[3-(3-fluoro-4-methoxy-phenyl)imidazo[1,2-a]pyrazin-8-yl]amino]-2,3,4,5-tetrahydro-2-benzazepin-1-one (18.7 mg) as a red solid. (ESI+) [(M+H)⁺]: 418

### Reference Example 270

### 2-[4-[8-[4-[4-(aminomethyl)piperidine-1-carbonyl]-3-methyl-anilino]imidazo[1,2-a]pyrazin-3-yl]-2,3-difluoro-phenoxy]-2-methyl-propanenitrile; Formic Acid

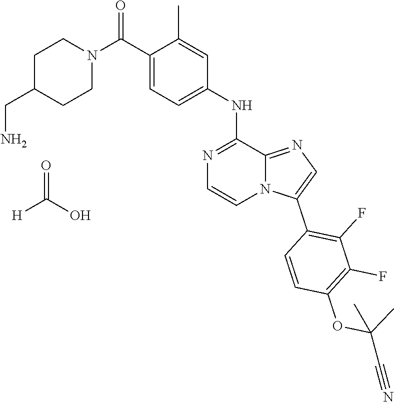 [SMILES: Cc1cc(Nc2nccn3c(-c4ccc(OC(C)(C)C#N)c(F)c4F)cnc23)ccc1C(=O)N1CCC(CN)CC1 ; O=CO]

### Step 1

### Ethyl 2-(4-bromo-2,3-difluoro-phenoxy)-2-methyl-propanoate

4-Bromo-2,3-difluorophenol (6 g, 28.7 mmol), ethyl 2-bromo-2-methylpropanoate (6.72 g, 34.5 mmol), Cs₂CO₃ (9.35 g, 28.7 mmol) and tetrabutylammoniumiodide (530 mg, 1.44 mmol) were suspended in DMF (30 mL). The resulting mixture was heated at 80° C. overnight. Then the mixture was cooled, diluted with water and extracted with ethyl ether. The combined organic phases were dried and concentrated. The residue was purified by flash column chromatography to give the title compound (6 g, 64.7% yield).

### Step 2

### 2-(4-bromo-2,3-difluoro-phenoxy)-2-methyl-propanoic Acid

Ethyl 2-(4-bromo-2,3-difluorophenoxy)-2-methyl-propanoate (4.3 g, 13.3 mmol) and NaOH (1.06 g, 26.6 mmol) was dissolved in a mixed solution of MeOH (36 mL), THF (18 mL) and water (12 mL). The reaction solution was stirred at room temperature for 2 h. Then the solution was acidified by 12 N HCl aqueous solution to pH 2-3. The water layer was extracted with ethyl acetate, dried over anhydrous MgSO₄ and concentrated to give the title compound as a white solid (3.5 g, 89.1% yield).

### Step 3

### 2-(4-bromo-2,3-difluoro-phenoxy)-2-methyl-propanamide

A mixture of 2-(4-bromo-2,3-difluorophenoxy)-2-methyl-propanoic acid (3.3 g, 11.2 mmol), 1-(3-dimethylaminopropyl)-3-ethylcarbodiimide hydrochloride (2.57 g, 13.4 mmol), 1-hydroxybenzotriazole (2.27 g, 16.8 mmol) and DIPEA (2.17 g, 2.93 mL, 16.8 mmol) in THF (30 mL) was stirred at room temperature for 2 h. Then aq. 25% NH₃ (10 mL) was added. The mixture was stirred overnight and then quenched with water. The aqueous layer was extracted with DCM. The combined organic layers were washed with saturated aq. NaHCO₃, brine, dried and concentrated. The residue was purified by flash column chromatography to give the title compound as a yellow solid (2 g, 60.8% yield).

### Step 4

### 2-(4-bromo-2,3-difluoro-phenoxy)-2-methyl-propanenitrile

To a solution of 2-(4-bromo-2,3-difluorophenoxy)-2-methyl-propanamide (2 g, 6.8 mmol) and Et₃N (4.13 g, 5.69 mL, 40.8 mmol) in dichloromethane (40 mL) was added trifluoroacetic anhydride (8.57 g, 5.76 mL, 40.8 mmol) at 0° C. After the addition, the solution was allowed to reach room temperature and stirred for 2 h. Then the mixture was heated at 70° C. overnight. The reaction was concentrated and diluted with water. The water phase was adjusted to pH 8-9 by NaHCO₃ aqueous solution. The water phase was extracted with DCM, dried and concentrated. The residue was used into next step reaction without further purification.

### Step 5

### 2-[2,3-difluoro-4-(4,4,5,5-tetramethyl-1,3,2-dioxaborolan-2-yl)phenoxy]-2-methyl-propanenitrile

A mixture of 4,4,4′,4′,5,5,5′,5′-octamethyl-2,2′-bi(1,3,2-dioxaborolane) (2.48 g, 9.78 mmol), 2-(4-bromo-2,3-difluorophenoxy)-2-methylpropanenitrile (1.8 g, 6.52 mmol), 1,1′-Bis(diphenylphosphino)ferrocene-palladium(II)dichloride dichloromethane complex (532 mg, 652 μmol) and potassium acetate (1.28 g, 13 mmol) in 1,4-dioxane (20 mL) was stirred at 80° C. overnight. Then the mixture was filtered and then concentrated. The residue was used in the next step reaction directly without further purification.

### Step 6

### Tert-butyl N-[[1-[4-[[3-[4-(1-cyano-1-methyl-ethoxy)-2,3-difluoro-phenyl]imidazo[1,2-a]pyrazin-8-yl]amino]-2-methyl-benzoyl]-4-piperidyl]methyl]carbamate

To a solution of tert-butyl ((1-(4-((3-iodoimidazo[1,2-a]pyrazin-8-yl)amino)-2-methylbenzoyl)piperidin-4-yl)methyl)carbamate (intermediate 73, 600 mg, 1.02 mmol) in water (5 mL) and THF (10 mL) was added 2-(2,3-difluoro-4-(4,4,5,5-tetramethyl-1,3,2-dioxaborolan-2-yl)phenoxy)-2-methylpropanenitrile (the crude product from step 5), 1,1′-Bis(diphenylphosphino)ferrocene-palladium(II) dichloride dichloromethane complex (166 mg, 203 μmol) and potassium phosphate tribasic (647 mg, 616 μl, 3.05 mmol) and then the mixture was degassed for 5 min with nitrogen and then stirred overnight at 70° C. The mixture was filtered. The solution was concentrated and the water layer was extracted with DCM. The organic layer was concentrated and the residue was purified by prep. HPLC to give the title compound (400 mg). MS (ESI, m/z): 660.3 [M+H]⁺

### Step 7

### 2-[4-[8-[4-[4-(aminomethyl)piperidine-1-carbonyl]-3-methyl-anilino]imidazo[1,2-a]pyrazin-3-yl]-2,3-difluoro-phenoxy]-2-methyl-propanenitrile Formate

tert-butyl ((1-(4-((3-(4-((2-cyanopropan-2-yl)oxy)-2,3-difluorophenyl)imidazo[1,2-a]pyrazin-8-yl)amino)-2-methylbenzoyl)piperidin-4-yl)methyl)carbamate (400 mg, 606 μmol) in TFA (5 mL) and DCM (10 mL) was stirred at room temperature for 2 h. Then the mixture was neutralized by NaHCO₃ aqueous solution. The water layer was extracted with DCM. The organic layer was dried and concentrated. The residue was purified by prep-HPLC to give the title compound as a white powder (120 mg). MS (ESI, m/z): 560.3 [M+H]⁺

### Intermediate 99

### 1-[2,3-Difluoro-4-(4,4,5,5-tetramethyl-1,3,2-dioxaborolan-2-yl)phenoxy]cyclopropanecarbonitrile

### Step 1

### Methyl 4-bromo-2-(4-bromo-2,3-difluoro-phenoxy)butanoate

K₂CO₃ (6.61 g, 47.8 mmol) was added into a solution of 4-bromo-2,3-difluorophenol (5 g, 23.9 mmol) in dry DMF (20 mL). The mixture was stirred at RT for 10 min. To the mixture was added methyl 2,4-dibromobutanoate (6.22 g, 23.9 mmol) dropwise. The resulting mixture was stirred at RT for 3 h. The mixture was diluted with ethyl acetate (60 mL), removed inorganic solid by filtration, then washed with water and brine. The organic phase was dried over flash column chromatography and concentrated. The residue was purified by flash column chromatography to give the title compound as an oil (4.7 g, 50% yield).

### Step 2

### Methyl 1-(4-bromo-2,3-difluoro-phenoxy)cyclopropanecarboxylate

Methyl 4-bromo-2-(4-bromo-2,3-difluorophenoxy)butanoate (4.7 g, 12.1 mmol) was dissolved in dry THF (30 mL) under N₂ protection, cooled with ice-acetone bath. To the mixture was added solid potassium tert-butoxide (1.36 g, 12.1 mmol) in portions. The resulting mixture was stirred at −10° C. for 30 min, then at room temperature for 2 h. The reaction was dried in vacuo, the residue was directly purified by flash column chromatography to afford the title compound (2 g, 53.8% yield).

### Step 3

### 1-[2,3-difluoro-4-(4,4,5,5-tetramethyl-1,3,2-dioxaborolan-2-yl)phenoxy]cyclopropanecarbonitrile

The title compound was prepared in similar procedures to the step 2, step 3, step 4 and step 5 of Reference Example 270 using methyl 1-(4-bromo-2,3-difluoro-phenoxy)cyclopropanecarboxylate as the starting materials.

### Intermediate 100

### 2-[[2,3-difluoro-4-(4,4,5,5-tetramethyl-1,3,2-dioxaborolan-2-yl)phenoxy]methyl]cyclopropanecarbonitrile

### Step 1

### Ethyl 2-(p-tolylsulfonyloxymethyl)cyclopropanecarboxylate

To a solution of ethyl 2-(hydroxymethyl)cyclopropane-1-carboxylate (4 g, 27.7 mmol) in DCM (30 mL) was added Et₃N (5.62 g, 7.73 mL, 55.5 mmol), DMAP (339 mg, 2.77 mmol) and 4-methylbenzenesulfonyl chloride (6.35 g, 33.3 mmol) at 0° C. The yellow reaction mixture was stirred for 3 hs at room temperature. Then the reaction mixture was poured on aqueous HCl (30 mL) and DCM (30 mL) and the layers were separated. The aqueous layer was extracted with DCM. The organic layer was concentrated and the residue was purified by flash column chromatography to give the title compound as an oil (4.7 g, 56.8% yield).

### Step 2

### Ethyl 2-[(4-bromo-2,3-difluoro-phenoxy)methyl]cyclopropanecarboxylate

4-bromo-2,3-difluorophenol (9 g, 43.1 mmol), ethyl 2-((tosyloxy)methyl)cyclopropane-1-carboxylate (12.8 g, 43.1 mmol) and Cs₂CO₃ (14 g, 43.1 mmol) was suspended in DMF (40 mL). The resulting mixture was heated at 65° C. overnight. Then the mixture was allowed to cool, diluted with water and extracted with ethyl ether. The combined organic phases were washed with Na₂CO₃ aqueous solution and concentrated. The residue was purified by flash column chromatography to give the title compound as an oil (8.5 g, 58.9% yield).

### Step 3

### 2-[[2,3-difluoro-4-(4,4,5,5-tetramethyl-1,3,2-dioxaborolan-2-yl)phenoxy]methyl]cyclopropanecarbonitrile

The title compound was prepared in similar procedures to the step 2, step 3, step 4 and step 5 of Reference Example 270 using ethyl 2-[(4-bromo-2,3-difluoro-phenoxy)methyl]cyclopropanecarboxylate as the starting materials.

### Intermediate 101

### 2-(3-fluoro-4-methylsulfanyl-phenyl)-4,4,5,5-tetramethyl-1,3,2-dioxaborolane

A mixture of 4,4,4′,4′,5,5,5′,5′-octamethyl-2,2′-bi(1,3,2-dioxaborolane) (2.54 g, 10 mmol), 4-bromo-2-fluoro-1-methylsulfanyl-benzene (2.2 g, 10 mmol), 1,1′-bis(diphenylphosphino)ferrocene-palladium(II)dichloride dichloromethane complex (653 mg, 0.8 mmol) and potassium acetate (1.96 g, 20 mmol) in 1,4-dioxane (20 mL) was stirred at 80° C. overnight. Then the mixture was poured into water and extracted with ethyl acetate. The organic layer was dried and concentrated. The residue was purified by flash column chromatography to afford the title compound as an oil (1.6 g, 61% yield).

### Reference Example 271

### (4-(aminomethyl)piperidin-1-yl)(4-((3-(2,5-difluoro-4-methoxyphenyl)imidazo[1,2-a]pyrazin-8-yl)amino)-2-methylphenyl)methanone

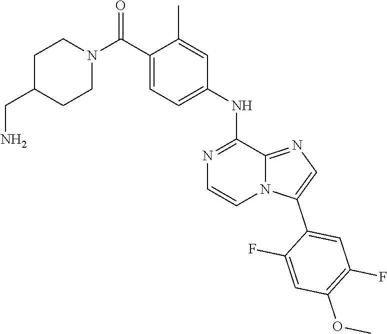 [SMILES: COc1cc(F)c(-c2cnc3c(Nc4ccc(C(=O)N5CCC(CN)CC5)c(C)c4)nccn23)cc1F]

### Step 1

### tert-butyl N-[[1-[4-[[3-(2,5-difluoro-4-methoxy-phenyl)imidazo[1,2-a]pyrazin-8-yl]amino]-2-methyl-benzoyl]-4-piperidyl]methyl]carbamate

To a solution of tert-butyl N-[[1-[4-[(3-iodoimidazo[1,2-a]pyrazin-8-yl)amino]-2-methyl-benzoyl]-4-piperidyl]methyl]carbamate (intermediate 73, 200 mg, 339 μmol) in water (2.5 mL) and THF (5 mL) was added (2,5-difluoro-4-methoxyphenyl)boronic acid (82.8 mg, 440 μmol), 1,1′-bis(diphenylphosphino)ferrocene-palladium(II)dichloride dichloromethane complex (55.3 mg, 67.7 μmol) and potassium phosphate tribasic (216 mg, 1.02 mmol). Then the mixture was degassed for 5 min with nitrogen and then stirred overnight at 80° C. After cooling to room temperature, the mixture was concentrated and DCM was added. The organic layer was dried over Na₂SO₄ and concentrated in vacuo. The residue was used into next step reaction directly. MS (ESI, m/z): 607 [M+H]⁺

### Step 2

### (4-(aminomethyl)piperidin-1-yl)(4-((3-(2,5-difluoro-4-methoxyphenyl)imidazo[1,2-a]pyrazin-8-yl)amino)-2-methylphenyl)methanone

To a solution of tert-butyl N-[[1-[4-[[3-(2,5-difluoro-4-methoxy-phenyl)imidazo[1,2-a]pyrazin-8-yl]amino]-2-methyl-benzoyl]-4-piperidyl]methyl]carbamate from step 1 in DCM (5 mL) was added CF₃COOH (5 mL). The mixture was stirred for 2 h at room temperature. Then the mixture was concentrated and NaHCO₃ aqueous solution was added to neutralize the solution to pH 8-9. The water phase was extracted with DCM. The organic phase was concentrated in vacuo and the residue was purified by prep-HPLC to afford the title compound (37 mg) as a solid. MS (ESI, m/z): 507.1 [M+H]⁺

The following examples were prepared in analogy to Reference Example 271:

| | | | MS | |
|---|---|---|---|---|
| | | | ESI | |
| Ex. | Name | Structure | [M + H]⁺ | Starting Material |
| REF 272 | (4- (aminomethyl)piperi- din-1-yl)(4-((3-(5- chloro-2-fluoro-4- methoxy- phenyl)imida- zo[1,2-a]pyrazin-8- yl)amino)-2- methylphenyl)metha- none | 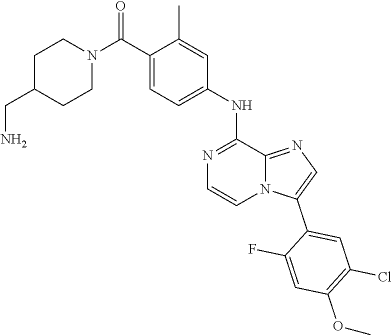 [SMILES: COc1cc(F)c(-c2cnc3c(Nc4ccc(C(=O)N5CCC(CN)CC5)c(C)c4)nccn23)cc1Cl] | 523.1 | Intermediate 73 and (5-chloro-2-fluoro-4- methoxyphenyl)bror- nic acid |
| REF 273 | (4- (aminomethyl)piperi- din-1-yl)(4-((3-(2,4- dichloro- phenyl)imida- zo[1,2-a]pyrazin-8- yl)amino)-2- methylphenyl)metha- none | 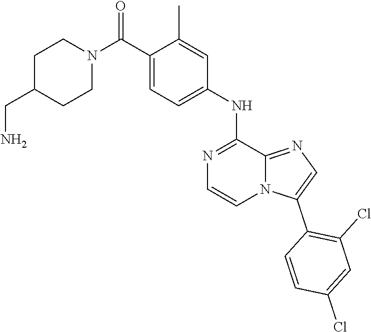 [SMILES: Cc1cc(Nc2nccn3c(-c4ccc(Cl)cc4Cl)cnc23)ccc1C(=O)N1CCC(CN)CC1] | 509.1 | Intermediate 73 and (2,4- dichlorophenyl)boro- nic acid |
| REF 274 | (4- (aminomethyl)piperi- din-1-yl)(4-((3-(2- chloro-4-methoxy- phenyl)imida- zo[1,2-a]pyrazin-8- yl)amino)-2- methylphenyl)metha- none | 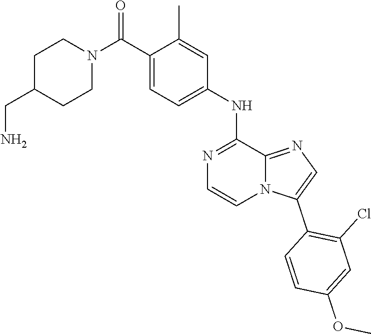 [SMILES: COc1ccc(-c2cnc3c(Nc4ccc(C(=O)N5CCC(CN)CC5)c(C)c4)nccn23)c(Cl)c1] | 505.1 | Intermediate 73 and (2-chloro-4- methoxyphenyl)boro- nic acid |
| REF 275 | (4- (aminomethyl)piperi- din-1-yl)(4-((3-(3- chloro-4-ethoxy-2- fluorophenyl)imidazo [1,2-a]pyrazin-8- yl)amino)-2- methylphenyl)metha- none formate | 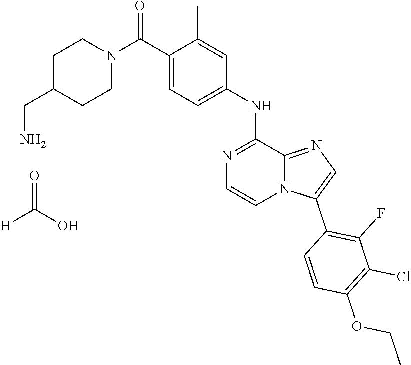 | 537.2 | Intermediate 73 and (3-chloro-4-ethoxy- 2- fluorophenyl)boronic acid |
| REF 276 | (4-(amino- methyl)piperi- din-1-yl)(2-methyl- 4-((3-(3,4,5-tri- fluorophenyl)imida- zo[1,2-a]pyrain-8- yl)amino)phenyl) methanone | 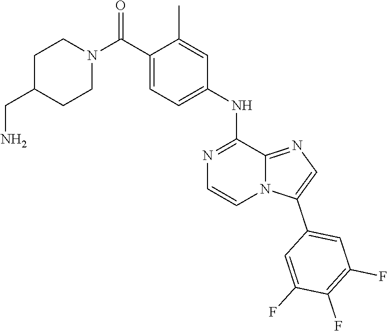 [SMILES: Cc1cc(Nc2nccn3c(-c4cc(F)c(F)c(F)c4)cnc23)ccc1C(=O)N1CCC(CN)CC1] | 495.3 | Intermediate 73 and (3,4,5- trifluorophenyl)boro- nic acid |
| 39 | (R)-1-(2-chloro-4- ((3-(2,3-difluoro-4- methoxyphe- nyl)imida- zo[1,2-a]pyrazin-8- yl)amino)benzoyl)- N-(pyrrolidin-3- yl)piperidine-4- carboxamide hydrochloride | 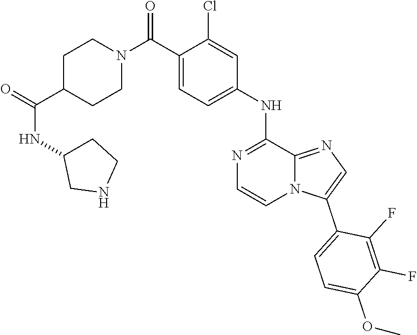 | 611.5 | Intermediate 74 and (2,3-difluoro- 4- methoxyphenyl)boro- nic acid |
| REF 277 | 1-(4-((3-(3-chloro-4- methoxyphe- nyl)imida- zo[1,2-a]pyrazin-8- yl)amino)-2- methylbenzoyl)-N- (2- (methylamino)ethyl) piperidine-4- carboxamide hydrochloride | 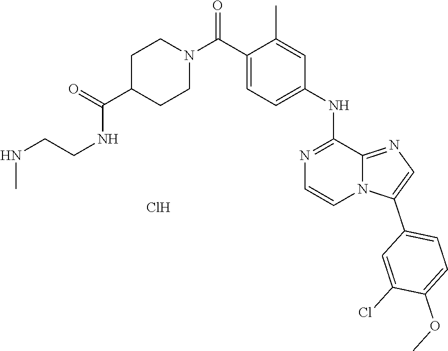 [SMILES: CNCCNC(=O)C1CCN(C(=O)c2ccc(Nc3nccn4c(-c5ccc(OC)c(Cl)c5)cnc34)cc2C)CC1 ; Cl] | 576.2 | Intermediate 102 and (3-chloro-4- methoxyphenyl)boro- nic acid |
| REF 278 | [4-(aminomethyl)-1- piperidyl]-[4-[[3- (2,3-dichloro-4- methoxy- phenyl)imidazo[1,2- a]pyrazin-8- yl]amino]-2-methyl- phenyl]methanone formate | 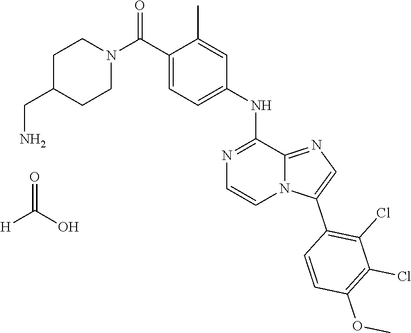 | 539.2 | Intermediate 73 and (2,3-dichloro-4- methoxyphenyl)boro- nic acid |
| REF 279 | 2-[[4-[8-[4-[4- (amino- methyl)piperi- dine-1-carbonyl]-3- methyl- anilino]imidazo[1,2- a]pyrazin-3-yl]-2,3- difluorophenoxy] methyl]cyclo- propanecarbonitrile formate | 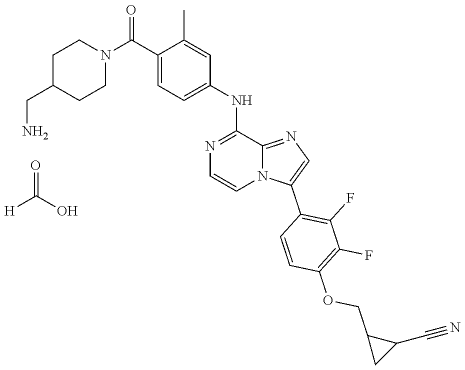 | 572.6 | Intermediate 73 and intermediate 100 |
| REF 280 | 1-[4-[8-[4-[4- (aminomethyl)piperi- dine-1-carbonyl]-3- methyl- anilino]imidazo[1,2- a]pyrazin-3-yl]-2,3- difluoro- phenoxy]cyclo- propane carbonitrile formate | 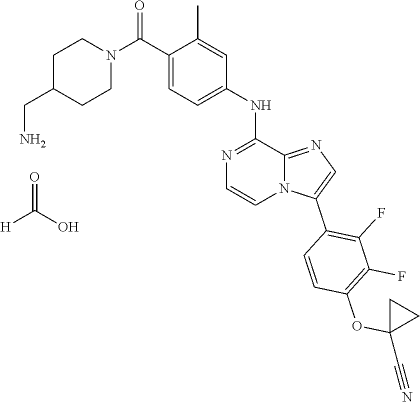 | 558.3 | Intermediate 73 and intermediate 99 |
| REF 281 | [4-(aminomethyl)-1- piperidyl]-[4-[[3-(3- fluoro-4- methylsulfanyl- phenyl)imidazo[1,2- a]pyrazin-8- yl]amino]-2-methyl- phenyl]methanone formate | 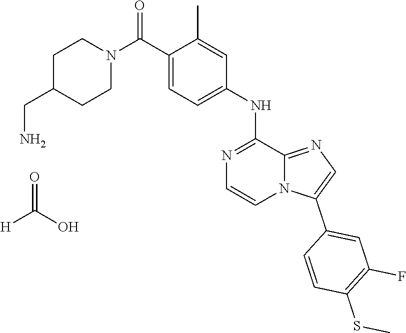 | 505.2 | Intermediate 73 and intermediate 101 |

### Reference Example 282

### [4-(aminomethyl)-1-piperidyl]-[4-[[3-[3-chloro-4-(cyclopropoxy)phenyl]imidazo[1,2-a]pyrazin-8-yl]amino]-2-methyl-phenyl]methanone Formate

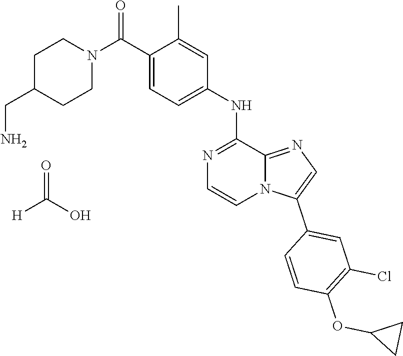 [SMILES: Cc1cc(Nc2nccn3c(-c4ccc(OC5CC5)c(Cl)c4)cnc23)ccc1C(=O)N1CCC(CN)CC1 ; O=CO]

### Step 1

### 4-bromo-2-chloro-1-(cyclopropoxy)benzene

Bromocyclopropane (8.75 g, 72.3 mmol) was added dropwise over 10 mins to a stirred solution of 4-bromo-2-chlorophenol (3 g, 14.5 mmol) and Cs₂CO₃ (11.8 g, 36.2 mmol) in dimethylacetamide (45 mL). The mixture was heated to 150° C. and stirred at this temperature for 16 h. Then the mixture was poured into water. The water layer was extracted with ethyl acetate. The combined organic layers were dried over anhydrous sodium sulphate and concentrated in vacuo. The residue was purified by flash column chromatography to give the title compound (3 g). MS (ESI, m/z): 247 [M+H]⁺

### Step 2

### 2-[3-chloro-4-(cyclopropoxy)phenyl]-4,4,5,5-tetramethyl-1,3,2-dioxaborolane

Under N₂ atmosphere, a mixture of 4,4,4′,4′,5,5,5′,5′-octamethyl-2,2′-bi(1,3,2-dioxaborolane) (3.69 g, 14.5 mmol), 4-bromo-2-chloro-1-cyclopropoxybenzene (3 g, 12.1 mmol), potassium acetate (2.38 g, 24.2 mmol) and 1,1′-bis(diphenylphosphino)ferrocene-palladium(II)dichloride dichloromethane complex (990 mg, 1.21 mmol) in 1,4-dioxane (50 mL) was stirred at 80° C. overnight. After cooling to room temperature, the mixture was concentrated and the residue was dissolved in DCM. The organic phase was washed with water, dried and concentrated. The residue was purified by flash column to give the title compound (2.6 g) as a solid.

### Step 3

### Tert-butyl N-[[1-[4-[[3-[3-chloro-4-(cyclopropoxy)phenyl]imidazo[1,2-a]pyrazin-8-yl]amino]-2-methyl-benzoyl]-4-piperidyl]methyl]carbamate

To a solution of tert-butyl N-[[1-[4-[(3-iodoimidazo[1,2-a]pyrazin-8-yl)amino]-2-methyl-benzoyl]-4-piperidyl]methyl]carbamate (300 mg, 508 μmol) in water (2.5 mL) and THF (5 mL) was added 2-(3-chloro-4-cyclopropoxyphenyl)-4,4,5,5-tetramethyl-1,3,2-dioxaborolane (195 mg, 660 μmol), 1,1′-bis(diphenylphosphino)ferrocene-palladium(II)dichloride dichloromethane complex (41.5 mg, 50.8 μmol) and potassium phosphate tribasic (324 mg, 308 μl, 1.52 mmol). Then the mixture was degassed for 5 min with nitrogen and then stirred overnight at 70° C. After cooling to room temperature, the mixture was concentrated. The water phase was extracted with DCM. The organic phase was dried and concentrated to give the crude product (300 mg). The crude product was used into next step reaction directly without further purification. MS (ESI, m/z): 631 [M+H]⁺

### Step 4

### (4-(aminomethyl)piperidin-1-yl)(4-((3-(3-chloro-4-cyclopropoxyphenyl)imidazo[1,2-a]pyrazin-8-yl)amino)-2-methylphenyl)methanone Formate

A solution of tert-butyl N-[[1-[4-[[3-[3-chloro-4-(cyclopropoxy)phenyl]imidazo[1,2-a]pyrazin-8-yl]amino]-2-methyl-benzoyl]-4-piperidyl]methyl]carbamate (300 mg, 475 μmol) in TFA (5 mL) and DCM (5 mL) was stirred at room temperature for 2 h. Then the solution was concentrated and the residue was diluted with water and DCM. The mixture solution was basified to pH 8-9 with K₂CO₃ aqueous solution. The water layer was extracted with DCM. The combined organic phases were dried and concentrated. The residue was purified by prep-HPLC to give the title compound (14 mg) as a solid. MS (ESI, m/z): 531.2 [M+H]⁺

### Reference Example 283

### (4-(aminomethyl)piperidin-1-yl)(4-((3-(4-(cyclopropylmethoxy)-3-fluorophenyl)imidazo[1,2-a]pyrazin-8-yl)amino)-2-methylphenyl)methanone

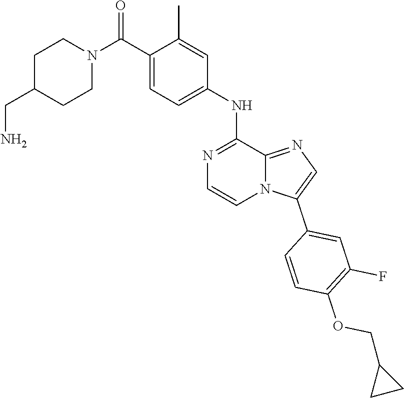 [SMILES: Cc1cc(Nc2nccn3c(-c4ccc(OCC5CC5)c(F)c4)cnc23)ccc1C(=O)N1CCC(CN)CC1]

The title compound was obtained in analogy to Reference Example 282 using (bromomethyl)cyclopropane instead of bromocyclopropane and 4-bromo-2-fluoro-phenol instead of 4-bromo-2-chlorophenol. MS (ESI, m/z): 529.3 [M+H]⁺

### Reference Example 284

### 2-[4-[8-[4-[4-(aminomethyl)piperidine-1-carbonyl]-3-methyl-anilino]imidazo[1,2-a]pyrazin-3-yl]-2,3-difluoro-phenoxy]propanenitrile;2,2,2-trifluoroacetic Acid

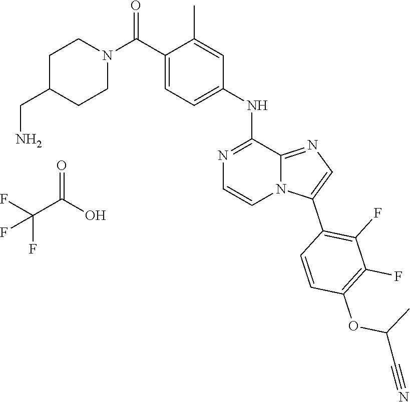 [SMILES: O=C(O)C(F)(F)F ; Cc1cc(Nc2nccn3c(-c4ccc(OC(C)C#N)c(F)c4F)cnc23)ccc1C(=O)N1CCC(CN)CC1]

The title compound was obtained in analogy to Reference Example 282 using 2-bromopropanenitrile instead of bromocyclopropane and 4-bromo-2,3-difluoro-phenol instead of 4-bromo-2-chlorophenol. MS (ESI, m/z): 546.5 [M+H]⁺

### Reference Example 285

### 4-(4-(8-((4-(4-(aminomethyl)piperidine-1-carbonyl)-3-methylphenyl)amino)imidazo[1,2-a]pyrazin-3-yl)-2,3-difluorophenoxy)butanenitrile

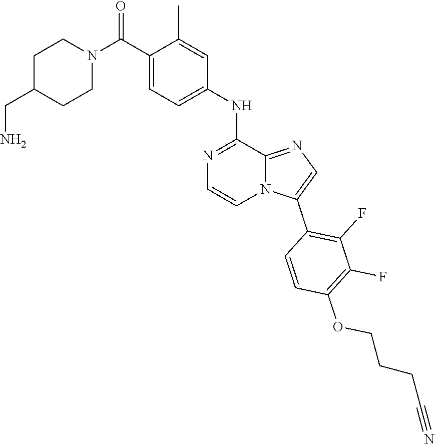 [SMILES: Cc1cc(Nc2nccn3c(-c4ccc(OCCCC#N)c(F)c4F)cnc23)ccc1C(=O)N1CCC(CN)CC1]

The title compound was obtained in analogy to Reference Example 282 using 4-bromobutanenitrile instead of bromocyclopropane and 4-bromo-2,3-difluoro-phenol instead of 4-bromo-2-chlorophenol. MS (ESI, m/z): 560.4 [M+H]⁺

### Reference Example 286

### [4-(aminomethyl)-1-piperidyl]-[4-[[3-(2,3-difluoro-4-prop-2-ynoxy-phenyl)imidazo[1,2-a]pyrazin-8-yl]amino]-2-methyl-phenyl]methanone; Formic Acid

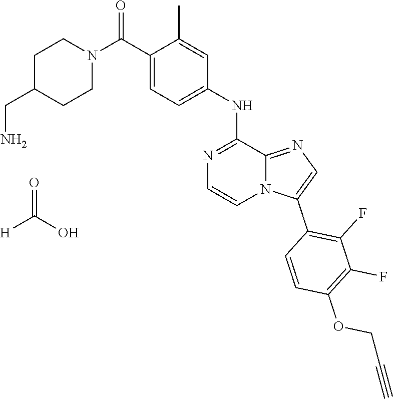 [SMILES: C#CCOc1ccc(-c2cnc3c(Nc4ccc(C(=O)N5CCC(CN)CC5)c(C)c4)nccn23)c(F)c1F ; O=CO]

The title compound was obtained in analogy to Reference Example 282 using 3-bromoprop-1-yne instead of bromocyclopropane and 4-bromo-2,3-difluoro-phenol instead of 4-bromo-2-chlorophenol. MS (ESI, m/z): 531.1 [M+H]⁺

### Reference Example 287

### [4-(aminomethyl)-1-piperidyl]-[4-[[3-[2,3-difluoro-4-(2-pyridyloxy)phenyl]imidazo[1,2-a]pyrazin-8-yl]amino]-2-methyl-phenyl]methanone Formate

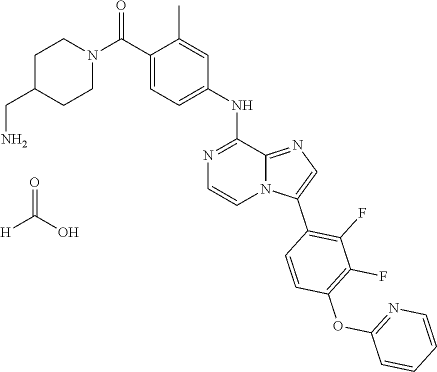 [SMILES: Cc1cc(Nc2nccn3c(-c4ccc(Oc5ccccn5)c(F)c4F)cnc23)ccc1C(=O)N1CCC(CN)CC1 ; O=CO]

### Step 1

### 2-(4-bromo-2,3-difluoro-phenoxy)pyridine

A mixture of 4-bromo-2,3-difluorophenol (3.5 g, 16.7 mmol), 2-fluoropyridine (2.44 g, 25.1 mmol) and K₂CO₃ (5.79 g, 41.9 mmol) in DMSO (20 mL) was heated at 120° C. for 3 days. Then the mixture was poured into water and extracted with DCM. The organic layer was dried and concentrated. The residue was purified by flash column chromatography to give the title compound as an oil (700 mg, 14.6% yield).

### Step 2

### 2-[2,3-difluoro-4-(4,4,5,5-tetramethyl-1,3,2-dioxaborolan-2-yl)phenoxy]pyridine

Under N₂, a mixture of 4,4,4′,4′,5,5,5′,5′-octamethyl-2,2′-bi(1,3,2-dioxaborolane) (746 mg, 2.94 mmol), 2-(4-bromo-2,3-difluorophenoxy)pyridine (0.7 g, 2.45 mmol), potassium acetate (480 mg, 4.89 mmol) and 1,1′-bis(diphenylphosphino)ferrocene-palladium(II)dichloride dichloromethane complex (200 mg, 245 μmol) in 1,4-dioxane (10 mL) was stirred at 80° C. overnight. The reaction solution was filtered and concentrated. The residue was used into next step reaction directly without further purification.

### Step 3

### Tert-butyl N-[[1-[4-[[3-[2,3-difluoro-4-(2-pyridyloxy)phenyl]imidazo[1,2-a]pyrazin-8-yl]amino]-2-methyl-benzoyl]-4-piperidyl]methyl]carbamate

A mixture of 2-(2,3-difluoro-4-(4,4,5,5-tetramethyl-1,3,2-dioxaborolan-2-yl)phenoxy)pyridine (the crude product from step 2), tert-butyl ((1-(4-((3-iodoimidazo[1,2-a]pyrazin-8-yl)amino)-2-methylbenzoyl)piperidin-4-yl)methyl)carbamate (intermediate 73, 400 mg, 677 μmol), potassium phosphate (288 mg, 1.35 mmol) and 1,1′-bis(diphenylphosphino)ferrocene-palladium(II)dichloride dichloromethane complex (55.3 mg, 67.7 μmol) in 1,4-dioxane (10 mL) and water (5 mL) was heated at 95° C. for 1 h in a microwave tube. Then the mixture was concentrated and the water layer was extracted with DCM. The organic layer was dried and concentrated. The residue was used into next step reaction directly without further purification.

### Step 4

### [4-(aminomethyl)-1-piperidyl]-[4-[[3-[2,3-difluoro-4-(2-pyridyloxy)phenyl]imidazo[1,2-a]pyrazin-8-yl]amino]-2-methyl-phenyl]methanone; Formic Acid

tert-butyl ((1-(4-((3-(2,3-difluoro-4-(pyridin-2-yloxy)phenyl)imidazo[1,2-a]pyrazin-8-yl)amino)-2-methylbenzoyl)piperidin-4-yl)methyl)carbamate (the crude product from step 3) was dissolved in DCM (5 mL) and TFA (5 mL). The solution was stirred for 1 h. Then the solution was concentrated and the residue was dissolved in DCM. Water (10 mL) was added. The solution was alkalized by addition of K₂CO₃ to pH 8-9. The water phase was extracted with DCM. The organic phase was concentrated and the residue was purified by prep-HPLC to give the title compound. MS (ESI, m/z): 570.2 [M+H]⁺

### Reference Example 288

### [4-(aminomethyl)-1-piperidyl]-[4-[[3-[2,3-difluoro-4-(4-pyridyloxy)phenyl]imidazo[1,2-a]pyrazin-8-yl]amino]-2-methyl-phenyl]methanone

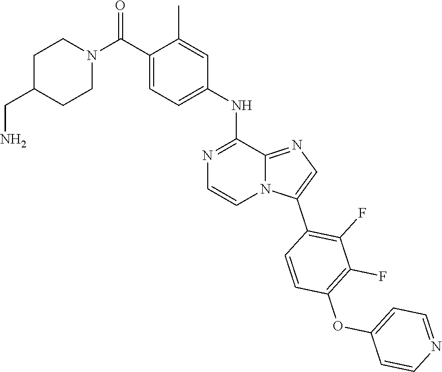 [SMILES: Cc1cc(Nc2nccn3c(-c4ccc(Oc5ccncc5)c(F)c4F)cnc23)ccc1C(=O)N1CCC(CN)CC1]

### Step 1

### 4-(4-bromo-2,3-difluoro-phenoxy)pyridine

4-bromo-2,3-difluorophenol (3.0 g, 14.4 mmol) in DMA (20 mL) was added potassium tert-butoxide (3.22 g, 28.7 mmol) at 0° C. The colorless solution was stirred for 1 h at room temperature. Then 4-fluoropyridine hydrochloride (1.92 g, 14.4 mmol) was added. The organic solution was heated at 100° C. overnight. The mixture was poured into water. The water layer was extracted with ethyl acetate. The combined organic layers were washed with saturated NaCl aqueous solution and concentrated. The residue was purified by flash column to give the title compound as an oil (3.3 g, 80% yield).

### Step 2

### [4-(aminomethyl)-1-piperidyl]-[4-[[3-[2,3-difluoro-4-(4-pyridyloxy)phenyl]imidazo[1,2-a]pyrazin-8-yl]amino]-2-methyl-phenyl]methanone

The title compound was obtained in similar procedures to step 2, step 3 and step 4 of Reference Example 287 using 4-(4-bromo-2,3-difluoro-phenoxy)pyridine as the starting material. MS (ESI, m/z): 570.2 [M+H]⁺

### Reference Example 289

### [4-(aminomethyl)-1-piperidyl]-[4-[[3-[2,3-difluoro-4-(3-pyridylmethoxy)phenyl]imidazo[1,2-a]pyrazin-8-yl]amino]-2-methyl-phenyl]methanone Formate

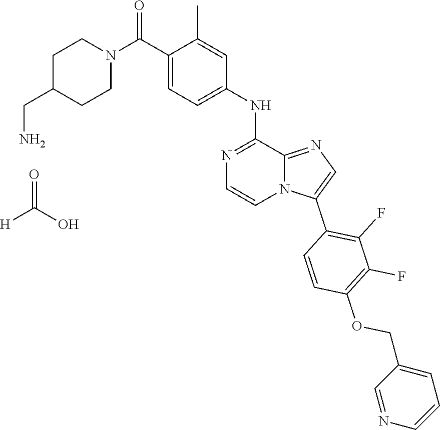

### Step 1: 3-[(4-bromo-2,3-difluoro-phenoxy)methyl]pyridine

4-bromo-2,3-difluorophenol (2.09 g, 10 mmol), 3-(chloromethyl)pyridine (1.64 g, 10 mmol), Cs₂CO₃ (3.25 g, 10 mmol) and tetrabutylammoniumiodide (185 mg, 0.5 mmol) was suspended in DMF (15 mL). The resulting mixture was heated to 60° C. overnight. Then the mixture was cooled, diluted with water and extracted with ethyl ether. The combined organic phases were dried and concentrated. The residue was purified by flash column chromatography to give the title compound as a yellow solid (1.6 g, 53% yield).

### Step 2

### 3-[[2,3-difluoro-4-(4,4,5,5-tetramethyl-1,3,2-dioxaborolan-2-yl)phenoxy]methyl]pyridine

Under N₂, a mixture of 4,4,4′,4′,5,5,5′,5′-octamethyl-2,2′-bi(1,3,2-dioxaborolane) (1.36 g, 5.3 mmol), 3-[(4-bromo-2,3-difluoro-phenoxy)methyl]pyridine (1.6 g, 5.3 mmol), potassium acetate (1.04 g, 10.6 mmol) and 1,1′-bis(diphenylphosphino)ferrocene-palladium(II)dichloride dichloromethane complex (346 mg, 0.424 mmol) in 1,4-dioxane (10 mL) was stirred at 80° C. overnight. The reaction solution was cooled and poured into water. The water phase was extracted with ethyl acetate. The organic phase was concentrated and purified by flash column chromatography to give the title compound as a solid (0.86 g).

### Step 3

### [4-(aminomethyl)-1-piperidyl]-[4-[[3-[2,3-difluoro-4-(3-pyridylmethoxy)phenyl]imidazo[1,2-a]pyrazin-8-yl]amino]-2-methyl-phenyl]methanone Formate

The title compound was prepared in analogy to Reference Example 271 using intermediate 73 and 3-[[2,3-difluoro-4-(4,4,5,5-tetramethyl-1,3,2-dioxaborolan-2-yl)phenoxy]methyl]pyridine as the starting materials. MS (ESI, m/z): 584.2 [M+H]⁺

### Reference Example 290

### [4-(aminomethyl)-1-piperidyl]-[4-[[3-[2,3-difluoro-4-(2-pyridylmethoxy)phenyl]imidazo[1,2-a]pyrazin-8-yl]amino]-2-methyl-phenyl]methanone;Formic Acid

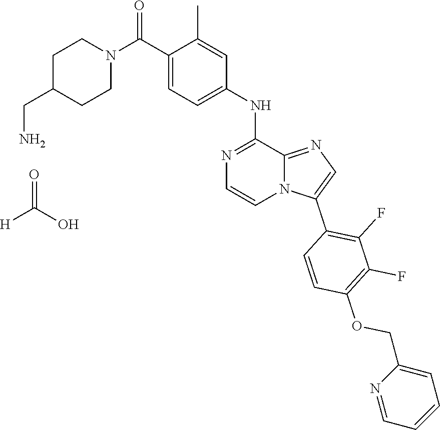

The title compound was obtained in analogy to Reference Example 289 using 2-(chloromethyl)pyridine instead of 3-(chloromethyl)pyridine. MS (ESI, m/z): 584.3 [M+H]⁺

### Reference Example 291

### [4-(aminomethyl)-1-piperidyl]-[4-[[3-(4-benzyloxy-2,3-difluoro-phenyl)imidazo[1,2-a]pyrazin-8-yl]amino]-2-methyl-phenyl]methanone Formate

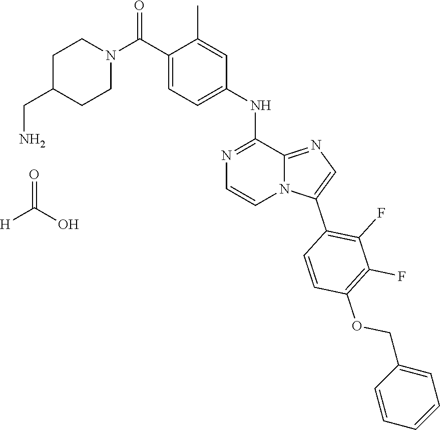

The title compound was obtained in analogy to Reference Example 289 using chloromethylbenzene instead of 3-(chloromethyl)pyridine. MS (ESI, m/z): 583.2 [M+H]⁺

### Reference Example 292

### [4-(aminomethyl)-1-piperidyl]-[4-[[3-[2,3-difluoro-4-(4-pyridylmethoxy)phenyl]imidazo[1,2-a]pyrazin-8-yl]amino]-2-methyl-phenyl]methanone Formate

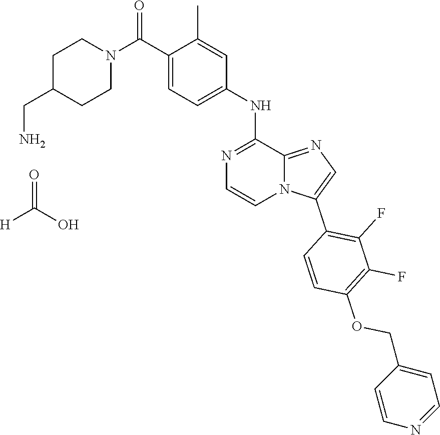

The title compound was obtained in analogy to Reference Example 289 using 4-(chloromethyl)pyridine instead of 3-(chloromethyl)pyridine. MS (ESI, m/z): 584.3 [M+H]⁺

### Reference Example 293

### [4-[[3-[4-(difluoromethoxy)phenyl]imidazo[1,2-a]pyrazin-8-yl]amino]-2-methyl-phenyl]-[4-(4-methylpiperazine-1-carbonyl)piperazin-1-yl]methanone Formate

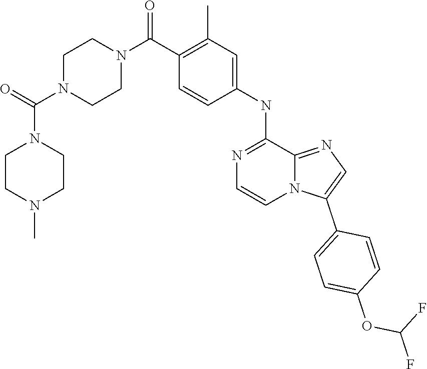 [SMILES: Cc1cc(Nc2nccn3c(-c4ccc(OC(F)F)cc4)cnc23)ccc1C(=O)N1CCN(C(=O)N2CCN(C)CC2)CC1]

To a solution of [4-[[3-[4-(difluoromethoxy)phenyl]imidazo[1,2-a]pyrazin-8-yl]amino]-2-methyl-phenyl]-piperazin-1-yl-methanone (Reference Example 294) (100.0 mg, 0.210 mmol, 1 eq.) in ACN (3 mL) was added N,N-diisopropylethylamine (0.11 mL, 0.630 mmol, 3 eq.) and N.N′-carbonyldiimidazole (37.28 mg, 0.230 mmol, 1.1 eq.), then the reaction was stirred at 20° C. for 3 h. 1-methylpiperazine (41.87 mg, 0.420 mmol, 2 eq) was added and then stirred at 80° C. for 12 h. After concentration, NMP (3 mL) was added and then stirred at 120° C. for 12 h. The reaction mixture was purified by prep-HPLC to give product [4-[[3-[4-(difluoromethoxy)phenyl]imidazo[1, 2-a]pyrazin-8-yl]amino]-2-methyl-phenyl]-[4-(4-methylpiperazine-1-carbonyl)piperazin-1-yl] methanone formate (25.1 mg, 0.040 mmol, 18% yield) as yellow solid.

LC-MS: [M+H]⁺: 605.2

### Reference Example 294

### [4-[[3-[4-(difluoromethoxy)phenyl]imidazo[1,2-a]pyrazin-8-yl]amino]-2-methyl-phenyl]-piperazin-1-yl-methanone Hydrochloride

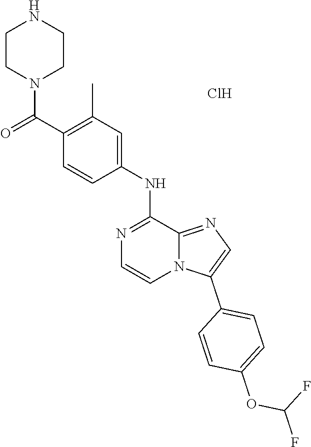 [SMILES: Cc1cc(Nc2nccn3c(-c4ccc(OC(F)F)cc4)cnc23)ccc1C(=O)N1CCNCC1 ; Cl]

### Step 1: tert-butyl 4-[4-[[3-[4-(difluoromethoxy)phenyl]imidazo[1,2-a]pyrazin-8-yl]amino]-2-methyl-benzoyl]piperazine-1-carboxylate

To a solution of 4-[[3-[4-(difluoromethoxy)phenyl]imidazo[1,2-a]pyrazin-8-yl]amino]-2-methyl-benzoic acid (Intermediate 7) (2.4 g, 5.84 mmol, 1 eq.) in DMF (20 mL) was added 1-BOC-piperazine (1.64 g, 8.78 mmol, 1.5 eq.), N,N-diisopropylethylamine (3.06 mL, 17.54 mmol, 3 eq.) and HATU (4.44 g, 11.7 mmol, 2 eq.), then the reaction was stirred at 25° C. for 12 h. 80 mL of water were added and extracted with ethyl acetate (3×100 mL). The combined organic layers were washed with brine (3×80 mL), dried over Na₂SO₄, filtered and concentrated. 40 mL of MTBE was added to the residue and stirred for 1 h. The suspension was filtered and dried to give tert-butyl 4-[4-[[3-[4-(difluoromethoxy)phenyl]imidazo[1,2-a]pyrazin-8-yl]amino]-2-methyl-benzoyl]piperazine-1-carboxylate (3.4 g, 5.88 mmol, 90% yield) as yellow solid.

LC-MS: [M+H]⁺: 579.3

### Step 2) Reference Example 294

### [4-[[3-[4-(difluoromethoxy)phenyl]imidazo[1,2-a]pyrazin-8-yl]amino]-2-methyl-phenyl]-piperazin-1-yl-methanone;hydrochloride

In a 150 mL round-bottomed flask, tert-butyl 4-(4-((3-(4-(difluoromethoxy)phenyl)imidazo[1,2-a]pyrazin-8-yl)amino)-2-methylbenzoyl)piperazine-1-carboxylate (Step 1) (3.087 g, 5.18 mmol, Eq: 1) was combined with dioxane (25 mL) to give a light brown suspension. Heating, sonicating and addition of 1.0 mL MeOH were necessary to get a proper solution. Then hydrogen chloride (4M solution in dioxane) (12.9 mL, 51.8 mmol, Eq: 10) was added slowly. Again 5 mL dioxane were added and the reaction mixture was stirred overnight. Diethylether was added, the suspension sonicated in an ultra sonic bath, filtered and washed with diethylether and dried in high vacuum, leading to the target compound as an off-white solid (2.7 g, yield: 100%). LC-MS: [M+H]⁺: 479.3

The following Examples and Intermediates were prepared in analogy to Reference Example 294:

| | | | MS | |
|---|---|---|---|---|
| | | | ESI | |
| Ex. | Name | Structure | [M + H]⁺ | Starting Material |
| REF 295 | [4-[[3-(3-fluoro-4- methoxy- phenyl)imidazo[1,2- a]pyrazin-8- yl]amino]-2-methyl- phenyl]-piperazin-1- yl-methanone hydrochoride | 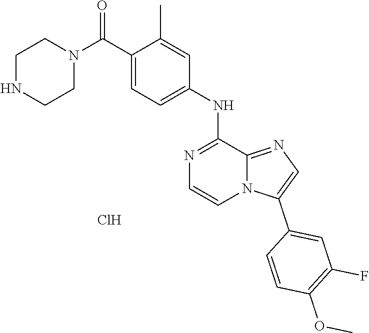 [SMILES: COc1ccc(-c2cnc3c(Nc4ccc(C(=O)N5CCNCC5)c(C)c4)nccn23)cc1F ; Cl] | 461.1 | Intermediate 6. After Step 1, purification by flash chromatography (silica gel, 50 g, 0% to 100% DCM/MeOH/NH4OH (95/5/1) |
| REF 296 | N-(2-aminoethyl)-4- [[3-(2,3-difluoro-4- methoxy- phenyl)imidazo[1,2- a]pyrazin-8- yl]amino]-2-methyl- benzamide; 2,2,2- trifluoroacetic acid | 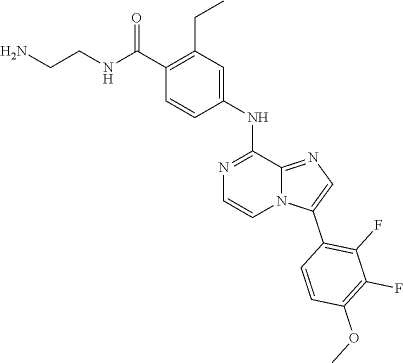 [SMILES: CCc1cc(Nc2nccn3c(-c4ccc(OC)c(F)c4F)cnc23)ccc1C(=O)NCCN] | 467.3 | Intermediate 86 and tert-butyl (2- aminoethyl)carbamate |
| REF 297 | N-(3-aminopropyl)- 4-[[3-(2,3-difluoro- 4-methoxy- phenyl)imidazo[1,2- a]pyrazin-8- yl]amino]-2-ethyl- benzamide; 2,2,2- trifluoroacetic acid | 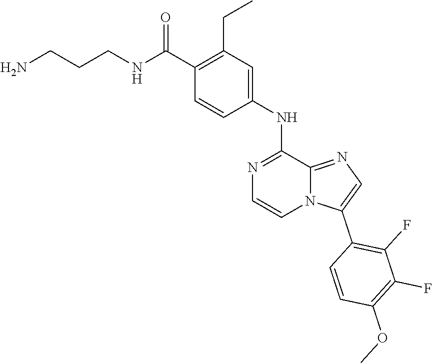 [SMILES: CCc1cc(Nc2nccn3c(-c4ccc(OC)c(F)c4F)cnc23)ccc1C(=O)NCCCN] | 481.4 | Intermediate 86 and tert-butyl (3- aminopropyl)carbamate |
| REF 298 | N-(3-aminopropyl)- 4-[[3-(2,3-difluoro- 4-methoxy- phenyl)imidazo[1,2- a]pyrazin-8- yl]amino]-2-ethyl- benzamide hydrochloride | 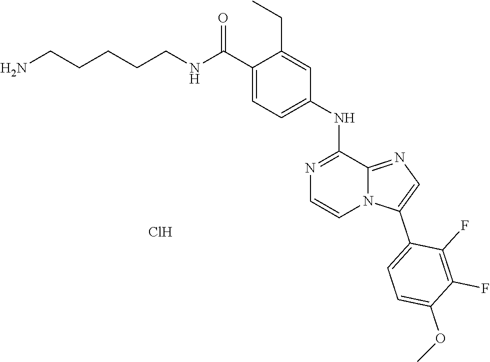 [SMILES: CCc1cc(Nc2nccn3c(-c4ccc(OC)c(F)c4F)cnc23)ccc1C(=O)NCCCCCN ; Cl] | 507.5 | Intermediate 86 and tert-butyl (5- aminopentyl)carbamate |
| 103 | [4-[[3-(2-chloro-3- fluoro-4-methoxy- phenyl)imidazo[1,2- a]pyrazin-8- yl]amino]-2-methyl- phenyl]piperazin-1- yl-methanone hydrochloride | 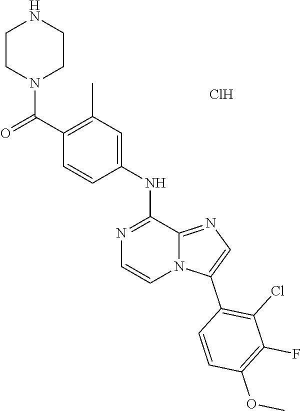 [SMILES: COc1ccc(-c2cnc3c(Nc4ccc(C(=O)N5CCNCC5)c(C)c4)nccn23)c(Cl)c1F ; Cl] | 495.1 | Intermediate 34 |
| 104 | 4-[[3-(2,3-difluoro- 4-methoxy- phenyl)imidazo[1,2- a]pyrazin-8- yl]amino]-2-ethyl-N- [(2R)-2- aminopropyl]benza- mide | 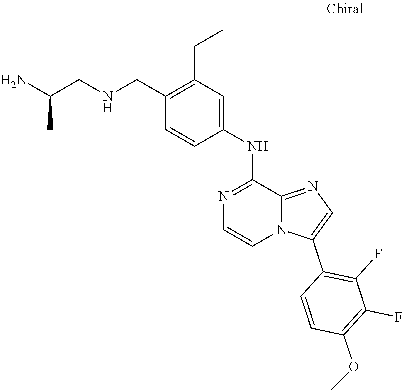 [SMILES: CCc1cc(Nc2nccn3c(-c4ccc(OC)c(F)c4F)cnc23)ccc1CNC[C@@H](C)N] | 481.3 | Intermediate 86 and tert-butyl N-[(1R)-2- amino-1-methyl- ethyl]carbamate |
| 105 | 4-[[3-(2,3-difluoro- 4-methoxy- phenyl)imidazo[1,2- a]pyrazin-8- yl]amino]-2-ethyl-N- [(2S)-2- aminopropyl]benza- mide | 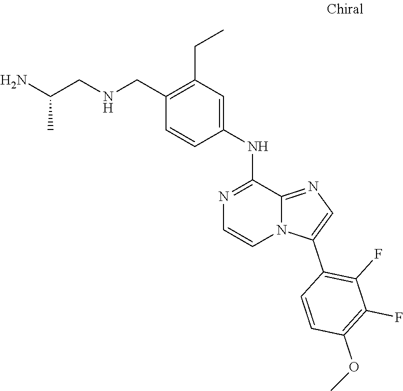 [SMILES: CCc1cc(Nc2nccn3c(-c4ccc(OC)c(F)c4F)cnc23)ccc1CNC[C@H](C)N] | 481.3 | Intermediate 86 and tert-butyl N-[(1S)-1- amino-1-methyl- ethyl]carbamate |

### Intermediate 86

### 4-[[3-(2,3-difluoro-4-methoxy-phenyl)imidazo[1,2-a]pyrazin-8-yl]amino]-2-ethyl-benzoic Acid

### Step 1) Methyl 4-((3-(2,3-difluoro-4-methoxyphenyl)imidazo[1,2-a]pyrazin-8-yl)amino)-2-ethylbenzoate

Under Ar, methyl 2-ethyl-4-((3-iodoimidazo[1,2-a]pyrazin-8-yl)amino)benzoate (Intermediate 42) (2 g, 4.36 mmol, Eq: 1) and (2,3-difluoro-4-methoxyphenyl)boronic acid [CAS #170981-41-6] (860 mg, 4.58 mmol, Eq: 1.05) were combined in dioxane (30 mL). A solution containing Na₂CO₃ [CAS #497-19-8] (1.02 g, 9.59 mmol, Eq: 2.2) in water (3 mL) was added and the off white suspension was degased with Ar. [1,1′-bis(diphenylphosphino)ferrocene]dichloropalladium(II), complex with dichloromethane [CAS #95464-05-4] (53.4 mg, 65.4 μmol, Eq: 0.015) was added and the orange suspension was stirred at 110° C. overnight. At RT, the suspension was filtered. The vessel and the filter cake were washed with ethyl acetate. Isolute was charged into the black suspension. The solvent was evaporated and the crude material was purified by flash chromatography (silica gel, 100 g, 0% to 50% ethyl acetate then 0% to 50% DCM/MeOH/25% aq.NH₃ (95:5:1) in DCM). The target compound was obtained as a light yellow solid (1.53 g, yield: 80%). LC-MS (ESP): m/z=439.3 [M+H]⁺.

### Step 2) Intermediate 86

### 4-[[3-(2,3-difluoro-4-methoxy-phenyl)imidazo[1,2-a]pyrazin-8-yl]amino]-2-ethyl-benzoic Acid

Under Ar, methyl 4-((3-(2,3-difluoro-4-methoxyphenyl)imidazo[1,2-a]pyrazin-8-yl)amino)-2-ethylbenzoate (Step 1) (0.765 g, 1.74 mmol, Eq: 1) was suspended in ethanol (9.79 mL). 1M LiOH solution (3.59 mL, 3.59 mmol, Eq: 2.06) was added and the reaction mixture was stirred at 80° C. overnight. The solvent was evaporated and the residue was partitioned between water (pH=12 with 1M NaOH) and ethyl acetate. The aqueous phase was acidified with 1M HCl to pH=1. The resulting off-white suspension was filtered and the cake was washed with water, leading to the target compound as an off-white solid (574 mg, yield: 78%). LC-MS (ESP): m/z=525.3 [M+H]⁺.

The following intermediates were prepared in analogy to Intermediate 86:

| | | MS ESI | |
|---|---|---|---|
| Int. | Name | [M + H]⁺ | Starting Material |
| 106 | 2-ethyl-4-((3-(3-fluoro-4- | 407.3 | Intermediate 42 and |
| | methoxyphenyl)imidazo[1,2-a]pyrazin-8- | | (3-fluoro-4- |
| | yl)amino)benzoic acid | | methoxyphenyl)boronic |
| | | | acid |
| 85 | 4-((3-(4-(difluoromethoxy)-2,3- | 461.3 | Intermediate 42 and |
| | difluorophenyl)imidazo[1,2-a]pyrazin-8- | | 107 |
| | yl)amino)-2-ethylbenzoic acid | | |
| 108 | 4-((3-(2,3-difluoro-4-m ethoxyphenyl)imidazo[1,2- | 411.3 | Intermediate 49 and |
| | a]pyrazin-8-yl)amino)-2-methylbenzoic acid | | (2,3-difluoro-4- |
| | | | methoxyphenyl)boronic |
| | | | acid |

### Intermediate 109

### 2-[2,3-difluoro-4-[8-[3-methyl-4-(piperazine-1-carbonyl)anilino]imidazo[1,2-a]pyrazin-3-yl]phenoxy]acetonitrile

### Step 1) tert-butyl 4-(4-((3-iodoimidazo[1,2-a]pyrazin-8-yl)amino)-2-methylbenzoyl)piperazine-1-carboxylate

In a 50 mL round-bottomed flask, 4-((3-iodoimidazo[1,2-a]pyrazin-8-yl)amino)-2-methylbenzoic acid (Intermediate 1) (1.880 g, 4.15 mmol, Eq: 1), tert-butyl piperazine-1-carboxylate [CAS #57260-71-6] (1.16 g, 6.22 mmol, Eq: 1.5) and HATU (3.15 g, 8.29 mmol, Eq: 2.0) were combined with DMF (20 mL) (fresh bottle) to give a skin colored emulsion. The reaction mixture was sonicated to break some of the remaining solids. The reaction mixture was stirred at room temperature and DIPEA (2.68 g, 3.62 mL, 20.7 mmol, Eq: 5.0) was added. Vigorous stirring at room temperature was continued for 2 h and then DMF was mostly evaporated in high vacuum at 50° C. The dark brown oil was diluted with DCM/MeOH (9:1) and charged with Isolute. Volatile solvents were evaporated in vacuum, remaining DMF was distilled off in HV at 50° C. The crude material was purified by flash chromatography (silica gel, 120 g, 0% to 100% DCM/MeOH/25% aq. NH₃ (95/5/1), solid loading), leading to tert-butyl 4-(4-((3-iodoimidazo[1,2-a]pyrazin-8-yl)amino)-2-methylbenzoyl)piperazine-1-carboxylate (2.449 g, 4.14 mmol, 99.7% yield) as a white solid. LC-MS (ESP): m/z=563.1 [M+H]⁺.

### Step 2) tert-butyl 4-(4-((3-(4-(cyanomethoxy)-2,3-difluorophenyl)imidazo[1,2-a]pyrazin-8-yl)amino)-2-methylbenzoyl)piperazine-1-carboxylate

In a 100 mL four-necked flask, tert-butyl 4-(4-((3-iodoimidazo[1,2-a]pyrazin-8-yl)amino)-2-methylbenzoyl)piperazine-1-carboxylate (obtained in Step 1) (1 g, 1.69 mmol, Eq: 1) was combined with 2-(2,3-difluoro-4-(4,4,5,5-tetramethyl-1,3,2-dioxaborolan-2-yl)phenoxy)acetonitrile (523 mg, 1.77 mmol, Eq: 1.05), sodium carbonate (394 mg, 3.72 mmol, Eq: 2.2) and dioxane (15 mL). The resulting suspension was stirred and sparged with argon for two minutes. Water (1.5 mL) was added and [1,1′-bis(diphenylphosphino)ferrocene]dichloropalladium(II), complex with dichloromethane [CAS #95464-05-4] (20.7 mg, 25.3 μmol, Eq: 0.015) was added thereafter. The reaction mixture was refluxed for 48 hrs under argon atmosphere. The reaction mixture was diluted with ethyl acetate, filtered and the vessel as well as the filter cake were washed with plenty ethyl acetate and the obtained black solution was concentrated in vacuum. The crude material was purified by flash chromatography (silica gel, 40 g, 40% ethyl acetate in heptane isocratic directly followed by 0%-50% DCM/MeOH/NH₃ (95/5/1), solid loading). The title compound tert-butyl 4-(4-((3-(4-(cyanomethoxy)-2,3-difluorophenyl)imidazo[1,2-a]pyrazin-8-yl)amino)-2-methylbenzoyl)piperazine-1-carboxylate (716 mg, 1.16 mmol, 68.8% yield) was obtained as a brown waxy solid. LC-MS (ESP): m/z=604.3 [M+H]+.

### Step 3) 2-[2,3-difluoro-4-[8-[3-methyl-4-(piperazine-1-carbonyl)anilino]imidazo[1,2-a]pyrazin-3-yl]phenoxy]acetonitrile

In a 50 mL round-bottomed flask, tert-butyl 4-(4-((3-(4-(cyanomethoxy)-2,3-difluorophenyl)imidazo[1,2-a]pyrazin-8-yl)amino)-2-methylbenzoyl)piperazine-1-carboxylate (obtained in Step 2) (716 mg, 1.19 mmol, Eq: 1) was combined with DCM (10 mL) to give a brown solution. TFA (1.48 g, 1 mL, 13 mmol, Eq: 10.9) was added and the reaction mixture was stirred at RT for 6 h and quenched with 5 mL of saturated aqueous sodium bicarbonate solution and 5 mL of water. Phases were separated and the separation funnel was washed with DCM/MeOH (9:1) to dissolve the precipitate. The organic phases were combined, dried with MgSO₄ monohydrate and filtered. The resulting light brown solution was evaporated in vacuo. The crude material was purified by flash chromatography (silica gel, 40 g, 0% to 100% DCM/MeOH/25% aq. NH₃ (90/10/1), solid loading) leading to 2-(2,3-difluoro-4-(8-((3-methyl-4-(piperazine-1-carbonyl)phenyl)amino)imidazo[1,2-a]pyrazin-3-yl)phenoxy)acetonitrile (417 mg, 812 μmol, 68.4% yield) as an off-white solid. LC-MS (ESP) m/z=504.2 [M+H]+.

The following intermediates were prepared in analogy to Intermediate 109:

| | | MS ESI | |
|---|---|---|---|
| Int. | Name | [M + H]⁺ | Starting Material |
| 110 | [4-[[3-(2,3-difluoro-4-methoxy- | 493.1 | Intermediate 63 and |
| | phenyl)imidazo[1,2-a]pyrazin-8-yl]amino]-2- | | (2,3-difluoro-4- |
| | ethyl-phenyl]-piperazin-1-yl-methanone | | methoxyphenyl)boronic |
| | | | acid (Step 2) |
| 111 | 2-[3-chloro-2-fluoro-4-[8-[3-methyl-4- | 520.2 | Intermediate 1 and 2-[3- |
| | (piperazine-1-carbonyl)anilino]imidazo[1,2- | | chloro-2-fluoro-4- |
| | a]pyrazin-3-yl]phenoxy]acetonitrile | | (4,4,5,5-tetramethyl- |
| | | | 1,3,2-dioxaborolan-2- |
| | | | yl)phenyl]acetonitrile |
| 112 | 2-[4-[8-[3-ethyl-4-(piperazine-1- | 518.3 | Intermediate 63 and 2- |
| | carbonyl)anilino]imidazo[1,2-a]pyrazin-3-yl]-2,3- | | [2,3-difluoro-4-(4,4,5,5- |
| | difluoro-phenoxy]acetonitrile | | tetramethyl-1,3,2- |
| | | | dioxaborolan-2- |
| | | | yl)phenoxy] acetonitrile |
| | | | (Step 2) |

### Intermediate 113

### Tert-butyl N-[2-[[2-ethyl-4-[(3-iodoimidazo[1,2-a]pyrazin-8 yl)amino]benzoyl]amino]ethyl]carbamate

A mixture of 2-ethyl-4-((3-iodoimidazo[1,2-a]pyrazin-8-yl)amino)benzoic acid hydrochloride (Intermediate 63) (4.45 g, 0.01 mol, Eq: 1), HATU (5.7 g, 15 mmol, Eq: 1.5) and DIPEA (6.46 g, 8.73 mL, 50 mmol, Eq: 5) in DMF (40 mL) was stirred for 15 min. at rt. Then tert-butyl (2-aminoethyl)carbamate (2.45 g, 2.41 mL, 15 mmol, Eq: 1.5) was added and the resulting solution was stirred at RT for 1½h. The reaction mixture was concentrated to dryness. To the liquid was added 100 mL H₂O and extracted with ethyl acetate. The organic layer was washed with brine, dried over MgSO₄ and evaporated to dryness. The crude product (7.48 g) was purified over 100 g SiO₂ 60 in DCM/DCM:MeOH 9:1 (0-100%) by flash chromatography. The obtained material (4.5 g) was triturated with 10 mL Et₂O. The mixture was stirred for ½ h, filtered, the solid washed with Et₂O and dried, yielding 3.57 g of the title compound as off-white solid (yield: 65%). MS (ESP) m/z=551.2 [M+H]+.

### Intermediate 102

### Tert-butyl (2-(1-(4-((3-iodoimidazo[1,2-a]pyrazin-8-yl)amino)-2-methylbenzoyl)piperidine-4-carboxamido)ethyl)(methyl)carbamate

The title compound was prepared in analogy to Intermediate 113 from Intermediate 68. LC/MS: [M+H]⁺=662.3

The following intermediate was prepared in analogy to Intermediate 113:

| | | MS ESI | |
|---|---|---|---|
| Int. | Name | [M + H]⁺ | Starting Material |
| 114 | tert-butyl N-[2-[[4-[(3-iodoimidazo[1,2- | 479.4 | Intermediate 1 and N- |
| | a]pyrazin-8-yl)amino]-2-methyl- | | BOC-ethylenediamine |
| | benzoyl]amino]ethyl]carbamate | | |
| 115 | tert-butyl N-[3-[[4-[(3-iodoimidazo[1,2- | 551.0 | Intermediate 1 and N- |
| | a]pyrazin-8-yl)amino]-2-methyl- | | BOC-1,3- |
| | benzoyl]amino]propyl]carbamate | | diaminopropane |

### Intermediate 116

### N-(2-aminoethyl)-4-[[3-[4-(cyanomethoxy)-2,3-difluoro-phenyl]imidazo[1,2-a]pyrazin-8-yl]amino]-2-ethyl-benzamide

### Step 1)

### Tert-butyl N-[2-[[4-[[3-[4-(cyanomethoxy)-2,3-difluoro-phenyl]imidazo[1,2-a]pyrazin-8-yl]amino]-2-ethyl-benzoyl]amino]ethyl]carbamate

To a solution of tert-butyl N-[2-[[2-ethyl-4-[(3-iodoimidazo[1,2-a]pyrazin-8-yl)amino]benzoyl]amino]ethyl]carbamate (Intermediate 113) (0.5 g, 0.910 mmol, 1 eq) in water (2 mL)/1,4-dioxane (20 mL) was added 2-[2,3-difluoro-4-(4,4,5,5-tetramethyl-1,3,2-dioxaborolan-2-yl)phenoxy]acetonitrile (0.8 g, 2.73 mmol, 3 eq), 1,1′-bis(diphenylphosphino)ferrocene-palladium(II)dichloride dichloromethane complex (0.04 g, 0.050 mmol, 0.050 eq) and sodium carbonate (0.19 g, 1.82 mmol, 2 eq) at 25° C., the mixture was stirred at 80° C. for 12 h. The mixture was poured into water (50 mL), and extracted with DCM (50 mL×3), the combined organic phases were washed with brine (50 mL×3), dried over anhydrous Na₂SO₄, and concentrated, the crude product was purified by flash column (PE:EA:DCM=1:1:1) to give tert-butyl N-[2-[[4-[[3-[4-(cyanomethoxy)-2,3-difluoro-phenyl]imidazo[1,2-a]pyrazin-8-yl]amino]-2-ethyl-benzoyl]amino]ethyl]carbamate (400 mg, 0.680 mmol, 74.43% yield) (PE:Ethyl acetate=1:1, Rf=0.1) as yellow solid. LC/MS: [M+H]⁺=592.3

### Step 2) Intermediate 116

### N-(2-aminoethyl)-4-[[3-[4-(cyanomethoxy)-2,3-difluoro-phenyl]imidazo[1,2-a]pyrazin-8-yl]amino]-2-ethyl-benzamide

To a solution of tert-butyl N-[2-[[4-[[3-[4-(cyanomethoxy)-2,3-difluoro-phenyl]imidazo[1,2-a]pyrazin-8-yl]amino]-2-ethyl-benzoyl]amino]ethyl]carbamate obtained in step 1 (200.0 mg, 0.340 mmol, 1 eq) in DCM (20 mL) was added trifluoroacetic acid (2.0 mL, 25.96 mmol, 76.79 eq) at 0° C., the mixture was stirred at 20° C. for 2 h. The mixture was concentrated to give N-(2-aminoethyl)-4-[[3-[4-(cyanomethoxy)-2,3-difluoro-phenyl]imidazo[1,2-a]pyrazin-8-yl]amino]-2-ethyl-benzamide (160 mg, 0.330 mmol, 96.3% yield) as a brown gum. LC/MS: [M+H]⁺=492.3

The following intermediates were prepared in analogy to Intermediate 116:

| | | MS ESI | |
|---|---|---|---|
| Int. | Name | [M + H]⁺ | Starting Material |
| 117 | N-(2-aminoethyl)-4-[[3-(2-chloro-3-fluoro- | 483.2 | Intermediate 113 and |
| | 4-methoxy-phenyl)imidazo[1,2-a]pyrazin- | | 2-(2-chloro-3-fluoro- |
| | 8-yl]amino]-2-ethyl-benzamide | | 4-methoxy-phenyl)- |
| | | | 4,4,5,5-tetramethyl- |
| | | | 1,3,2-dioxaborolane |
| 118 | N-(2-aminoethyl)-4-[[3-[2-chloro-4- | 508.2 | Intermediate 113 and |
| | (cyanomethoxy)-3-fluoro- | | Intermediate 55: 2-[3- |
| | phenyl]imidazo[1,2-a]pyrazin-8- | | chloro-2-fluoro-4- |
| | yl]amino]-2-ethyl-benzamide | | (4,4,5,5-tetramethyl- |
| | | | 1,3,2-dioxaborolan-2- |
| | | | yl)phenoxy]acetonitrile |
| 119 | N-(2-aminoethyl)-4-[[3-(4-chloro-2,3- | 471.2 | Intermediate 113 and |
| | difluoro-phenyl)imidazo[1,2-a]pyrazin-8- | | 2-(4-chloro-2,3- |
| | yl]amino]-2-ethyl-benzamide | | difluoro-phenyl)- |
| | | | 4,4,5,5-tetramethyl- |
| | | | 1,3,2-dioxaborolane |
| 120 | N-(2-aminoethyl)-4-[[3-[4- | 503.2 | Intermediate 113 and |
| | (difluoromethoxy)-2,3-difluoro- | | Intermediate 107 |
| | phenyl]imidazo[1,2-a]pyrazin-8- | | |
| | yl]amino]-2-ethyl-benzamide | | |
| 121 | N-(2-aminoethyl)-4-[[3-[4- | 478.2 | Intermediate 114 and |
| | (cyanomethoxy)-2,3-difluoro- | | 2-[2,3-difluoro-4- |
| | phenyl]imidazo[1,2-a]pyrazin-8- | | (4,4,5,5-tetramethyl- |
| | yl]amino]-2-methyl-benzamide | | 1,3,2-dioxaborolan-2- |
| | | | yl)phenoxy]acetonitrile |
| 122 | N-(3-aminopropyl)-4-[[3-[4- | 492.2 | Intermediate 115 and |
| | (cyanomethoxy)-2,3-difluoro- | | 2-[2,3-difluoro-4- |
| | phenyl]imidazo[1,2-a]pyrazin-8- | | (4,4,5,5-tetramethyl- |
| | yl]amino]-2-methyl-benzamide | | 1,3,2-dioxaborolan-2- |
| | | | yl)phenoxy]acetonitrile |

### Intermediate 123

### (2-chloro-4-((3-(3-fluoro-4-methoxyphenyl)imidazo[1,2-a]pyrazin-8-yl)amino)phenyl)(piperazin-1-yl)methanone hydrochloride

The title compound was prepared in analogy to Intermediate REF 15 from Intermediate 2 and tert-butyl piperazine-1-carboxylate.

MS obsd. (ESI⁻) [(M−H)]⁻: 479.4

### Reference Example 299

### N-(2-aminoethyl)-4-[4-[[3-[4-(difluoromethoxy)phenyl]imidazo[1,2-a]pyrazin-8-yl]amino]-2-methyl-benzoyl]piperazine-1-carboxamide Hydrochloride

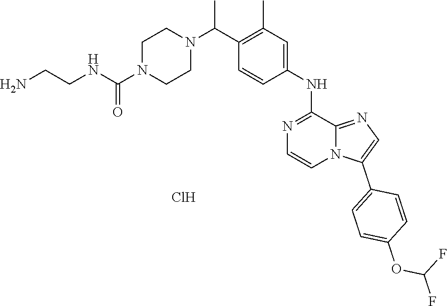 [SMILES: Cc1cc(Nc2nccn3c(-c4ccc(OC(F)F)cc4)cnc23)ccc1C(C)N1CCN(C(=O)NCCN)CC1 ; Cl]

### Step 1)

To a solution of [4-[[3-[4-(difluoromethoxy)phenyl]imidazo[1,2-a]pyrazin-8-yl]amino]-2-methyl-phenyl]-piperazin-1-yl-methanone (Reference Example 294) (100.0 mg, 0.210 mmol, 1 eq) in DCM (5 mL) was added N,N-diisopropylethylamine (0.18 mL, 1.04 mmol, 5 eq) and bis(trichloromethyl)carbonate (24.81 mg, 0.080 mmol, 0.400 eq) at 0° C., the mixture was stirred at 0° C. for 1 h, then N-BOC-ethylenediamine (100.45 mg, 0.630 mmol, 3 eq) was added, the mixture was stirred at 25° C. for 12 h, LC-MS showed the reaction was completed. The mixture was concentrated and purified by prep-HPLC (TFA) to give tert-butyl N-[2-[[4-[4-[[3-[4-(difluoromethoxy)phenyl]imidazo[1,2-a]pyrazin-8-yl]amino]-2-methyl-benzoyl]piperazine-1-carbonyl]amino]ethyl]carbamate (80 mg, 0.120 mmol, 57.59% yield) as a yellow solid.

LC-MS: [M+H]⁺: 665.3

### Step 2)

To a solution of tert-butyl N-[2-[[4-[4-[[3-[4-(difluoromethoxy)phenyl]imidazo [1,2-a]pyrazin-8-yl]amino]-2-methyl-benzoyl]piperazine-1-carbonyl]amino]ethyl]carbamate (obtained in step 1) (80.0 mg, 0.120 mmol, 1 eq) in methanol (20 mL) was added hydrochloric acid in MeOH (0.77 mL, 3.1 mmol, 25.72 eq) and then stirred at 25° C. for 2 h. LC-MS showed the reaction was complete. After concentration, 100 mL of saturated NaHCO₃ aqueous were added and extracted with DCM (100 mL×3). The combined organic layers were washed with brine (100 mL), dried over Na₂SO₄, filtered and concentrated. The residue was purified by prep-HPLC (HCl) to give N-(2-aminoethyl)-4-[4-[[3-[4-(difluoromethoxy)phenyl]imidazo[1,2-a]pyrazin-8-yl]amino]-2-methyl-benzoyl]piperazine-1-carboxamide hydrochloride (28 mg, 0.040 mmol, 36.24% yield) as yellow solid.

LC-MS: [M+H]⁺: 565.1

The following examples were prepared in analogy to Reference Example 299 (Step 2 only required if protecting group needs removal):

| | | | MS | |
|---|---|---|---|---|
| | | | ESI | |
| | | | [M + | |
| Ex. | Name | Structure | H]⁺ | Starting Material |
| REF 300 | [4-[[3-[4- (difluoromethoxy)phe- nyl]imidazo[1,2- a]pyrazin-8- yl]amino]-2-methyl- phenyl]-[4-[2- [(dimethylamino)meth- yl]morpholine-4- carbonyl]piperazin- 1-yl]methanone | 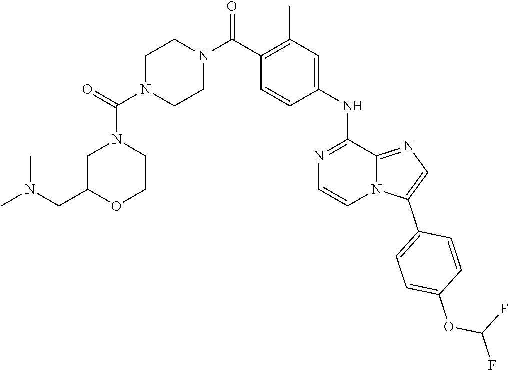 [SMILES: Cc1cc(Nc2nccn3c(-c4ccc(OC(F)F)cc4)cnc23)ccc1C(=O)N1CCN(C(=O)N2CCOC(CN(C)C)C2)CC1] | 649.3 | Reference Example 294 and N,N-dimethyl-2- morpholinmethanamine |
| REF 301 | [4-[[3-[4- (difluoromethoxy)phe- nyl]imidazo[1,2- a]pyrazin-8- yl]amino]-2-methyl- phenyl]-[4-[3- (hydroxymethyl)piper- azine-1- carbonyl]piperazin- 1-yl]methanone hydrochloride | 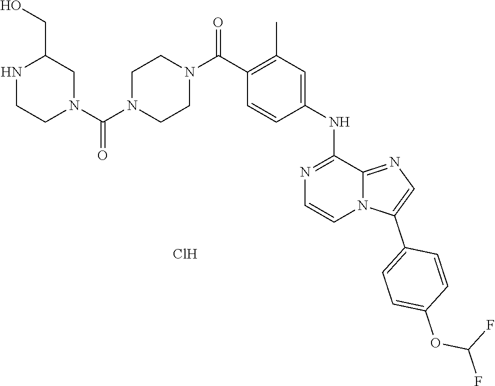 [SMILES: Cc1cc(Nc2nccn3c(-c4ccc(OC(F)F)cc4)cnc23)ccc1C(=O)N1CCN(C(=O)N2CCNC(CO)C2)CC1 ; Cl] | 621.3 | Reference Example 294 and tert-butyl 2-(hydroxy- methyl)piperazine- 1-carboxylate |
| REF 302 | [4-[2- (aminomethyl)morpho- line-4- carbonyl]piperazin- 1-yl]-[4-[[3-[4- (difluoromethoxy)phe- nyl]imidazo[1,2- a]pyrazin-8- yl]amino]-2-methyl- phenyl]methanone hydrochloride | 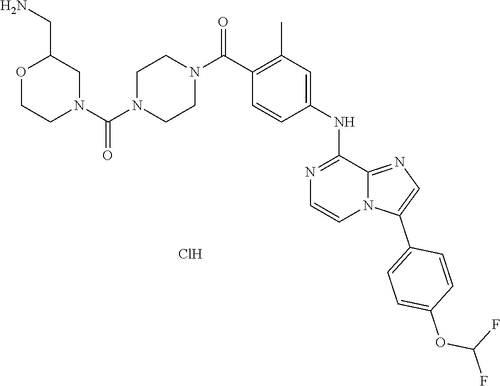 [SMILES: Cc1cc(Nc2nccn3c(-c4ccc(OC(F)F)cc4)cnc23)ccc1C(=O)N1CCN(C(=O)N2CCOC(CN)C2)CC1 ; Cl] | 621.2 | Reference Example 294 and tert-butyl N-(morpholin-2- ylmethyl)carbamate |
| REF 303 | 4-[4-[[3-[4- (difluoromethoxy)phe- nyl]imidazo[1,2- a]pyrazin-8- yl]amino]-2-methyl- benzoyl]-N,N- dimethyl-piperazine-1- carboxamide | 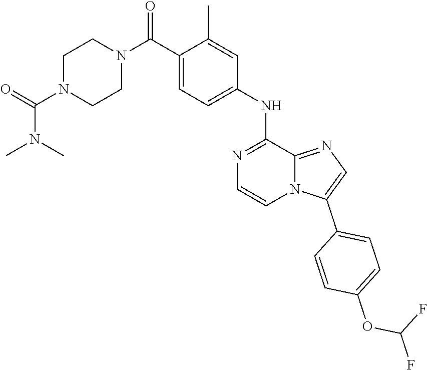 [SMILES: Cc1cc(Nc2nccn3c(-c4ccc(OC(F)F)cc4)cnc23)ccc1C(=O)N1CCN(C(=O)N(C)C)CC1] | 550.2 | Reference Example 294 and dimethylamine |
| REF 304 | [4-[[3-[4- (difluoromethoxy)phe- nyl]imidazo[1,2- a]pyrazin-8- yl]amino]-2-methyl- phenyl]-[4-[2- (methylaminomethyl) morpholine-4- carbonyl]piperazin- 1-yl]methanone hydrochloride | 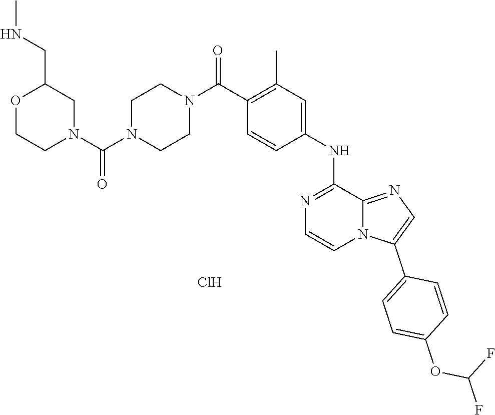 [SMILES: CNCC1CN(C(=O)N2CCN(C(=O)c3ccc(Nc4nccn5c(-c6ccc(OC(F)F)cc6)cnc45)cc3C)CC2)CCO1 ; Cl] | 635.1 | Reference Example 294 and Carbamic acid, methyl(2- morpholinylmethyl)-, 1,1-dimethylethyl ester [CAS# 185692-04-0] |
| REF 305 | 2-[2,3-difluoro-4-[8- [3-methyl-4-[4- (piperazine-1- carbonyl)piperazine-1- carbonyl]anilino]imi- dazo[1,2-a]pyrazin- 3-yl]phe- noxy]acetonitrile formate | 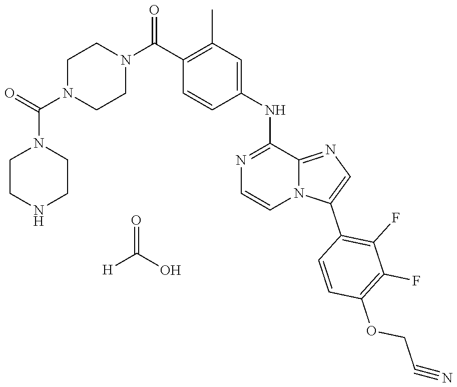 [SMILES: Cc1cc(Nc2nccn3c(-c4ccc(OCC#N)c(F)c4F)cnc23)ccc1C(=O)N1CCN(C(=O)N2CCNCC2)CC1 ; O=CO] | 616.2 | Intermediate 109 and tert-butyl piperazine-1- carboxylate [CAS#57260-71-6]. Purification: prep HPLC with formic acid |
| REF 306 | 4-[4-[[3-[4- (difluoromethoxy)phe- nyl]imidazo[1,2- a]pyrazin-8- yl]amino]-2-methyl- benzoyl]-N-[(3S)- pyrrolidin-3- yl]piperazine-1- carboxamide hydrochloride | 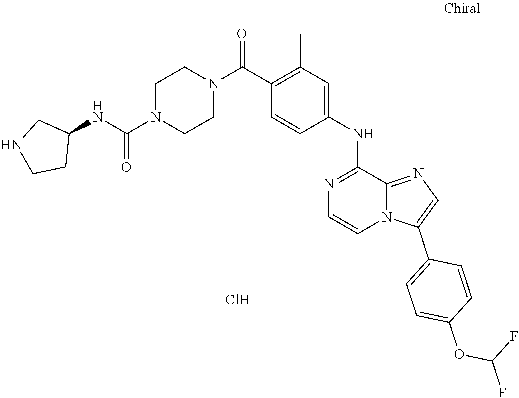 [SMILES: Cc1cc(Nc2nccn3c(-c4ccc(OC(F)F)cc4)cnc23)ccc1C(=O)N1CCN(C(=O)N[C@H]2CCNC2)CC1 ; Cl] | 591.1 | Reference Example 294 and (S)-3-amino-1-N- BOC-pyrrolidine |
| REF 307 | N-(2-aminoethyl)-4- [4-[[3-[4- (cyanomethoxy)-2,3- difluoro- phenyl]imidazo[1,2- a]pyrazin-8- yl]amino]-2-methyl- benzoyl]piperazine- 1-carboxamide formate | 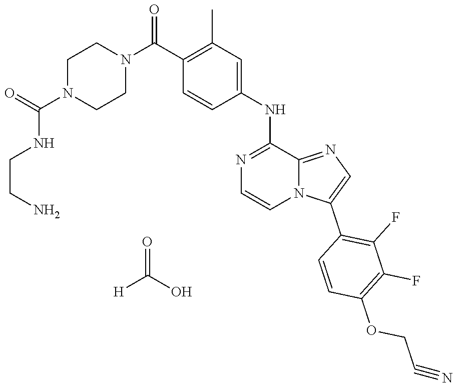 [SMILES: Cc1cc(Nc2nccn3c(-c4ccc(OCC#N)c(F)c4F)cnc23)ccc1C(=O)N1CCN(C(=O)NCCN)CC1 ; O=CO] | 590.2 | Intermediate 109 and N- BOC-ethylenediamine Purification: prep HPLC with formic acid |
| REF 308 | [4-[(2R)-2- (aminomethyl)morpho- line-4- carbonyl]piperazin- 1-yl]-[4-[[3-(2,3- difluoro-4-methoxy- phenyl)imidazo[1,2- a]pyrazin-8- yl]amino]-2-methyl- phenyl]methanone formate | 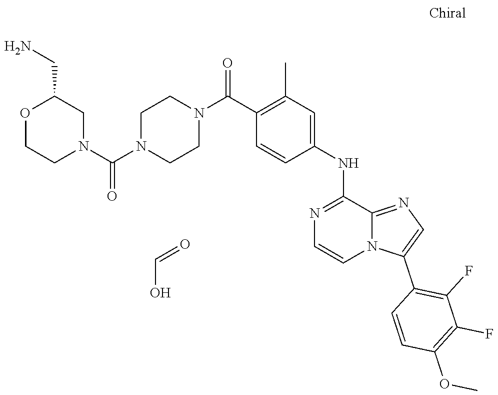 [SMILES: COc1ccc(-c2cnc3c(Nc4ccc(C(=O)N5CCN(C(=O)N6CCO[C@H](CN)C6)CC5)c(C)c4)nccn23)c(F)c1F ; O=CO] | 621.2 | Reference Example 208 and tert-butyl N-[[(2S)- morpholin-2- yl]methyl]carbamate Purification: prep HPLC with formic acid |
| REF 309 | 4-[4-[[3-[4- (difluoromethoxy)phe- nyl]imidazo[1,2- a]pyrazin-8- yl]amino]-2-methyl- benzoyl]-N-[(3R)- pyrrolidin-3- yl]piperazine-1- carboxamide hydrochloride | 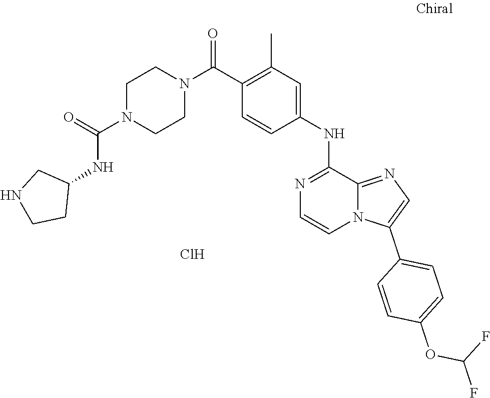 [SMILES: Cc1cc(Nc2nccn3c(-c4ccc(OC(F)F)cc4)cnc23)ccc1C(=O)N1CCN(C(=O)N[C@@H]2CCNC2)CC1 ; Cl] | 591.3 | Reference Example 294 and (R)-(+)-1-BOC-3- aminopyrrolidine |
| REF 310 | 4-[4-[[3-[4- (difluoromethoxy)phe- nyl]imidazo[1,2- a]pyrazin-8- yl]amino]-2-methyl- benzoyl]-N-(4- piperidyl)piperazine- 1-carboxamide hydrochloride | 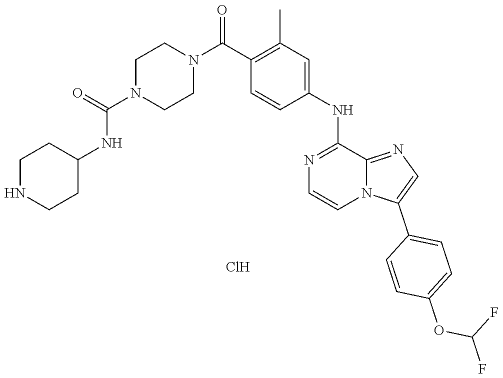 [SMILES: Cc1cc(Nc2nccn3c(-c4ccc(OC(F)F)cc4)cnc23)ccc1C(=O)N1CCN(C(=O)NC2CCNCC2)CC1 ; Cl] | 605.3 | Reference Example 294 and 4-amino-1-boc- piperidine |
| REF 311 | N-(azetidin-3- ylmethyl)-4-[4-[[3- [4- (difluoromethoxy)phe- nyl]imidazo[1,2- a]pyrazin-8- yl]amino]-2-methyl- benzoyl]piperazine- 1-carboxamide formate | 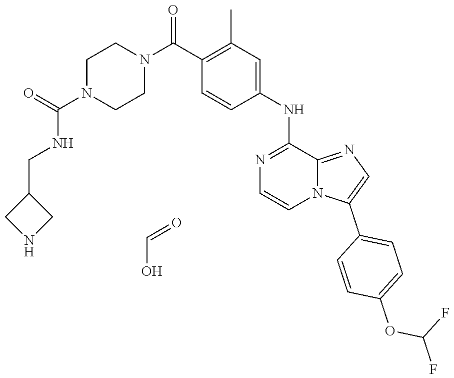 [SMILES: Cc1cc(Nc2nccn3c(-c4ccc(OC(F)F)cc4)cnc23)ccc1C(=O)N1CCN(C(=O)NCC2CNC2)CC1 ; O=CO] | 591.1 | Reference Example 294 and 1-BOC-3- (aminomethyl) azetidine. Purification: prep HPLC with formic acid |
| REF 312 | [4-[(2S)-2- (aminomethyl)morpho- line-4- carbonyl]piperazin- 1-yl]-[4-[[3-(2,3- difluoro-4-methoxy- phenyl)imidazo[1,2- a]pyrazin-8- yl]amino]-2-methyl- phenyl]methanone formate | 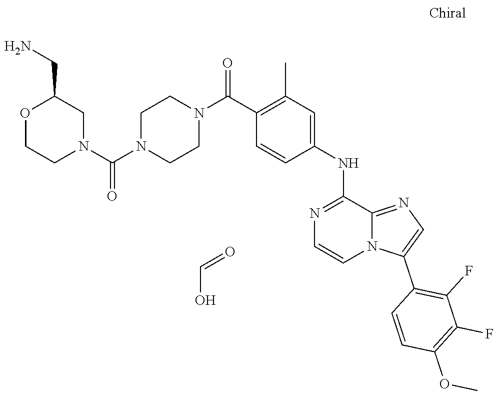 [SMILES: COc1ccc(-c2cnc3c(Nc4ccc(C(=O)N5CCN(C(=O)N6CCO[C@@H](CN)C6)CC5)c(C)c4)nccn23)c(F)c1F ; O=CO] | 621.3 | Reference Example 208 and 1,1-Dimethylethyl [(2R)-2- morpholinylmethyl]carba- mate [CAS# 186202- 57-3]. Purification: prep HPLC with formic acid |
| REF 313 | [4-[(2S)-2- (aminomethyl)morpho- line-4- carbonyl]piperazin- 1-yl]-[4-[[3-[4- (difluoromethoxy)phe- nyl]imidazo[1,2- a]pyrazin-8- yl]amino]-2-methyl- phenyl]methanone formate | 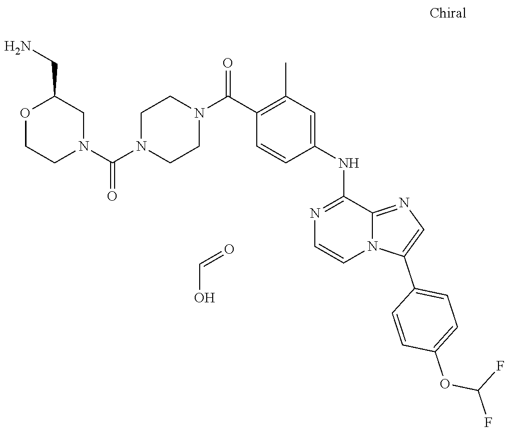 [SMILES: Cc1cc(Nc2nccn3c(-c4ccc(OC(F)F)cc4)cnc23)ccc1C(=O)N1CCN(C(=O)N2CCO[C@@H](CN)C2)CC1 ; O=CO] | 621.3 | Reference Example 294 and 1,1-Dimethylethyl [(2R)-2- morpholinylmethyl]carba- mate [CAS# 186202- 57-3]. Purification: prep HPLC with formic acid |
| REF 314 | N-[(1S)-2-amino-1- methyl-ethyl]-4-[4- [[3-(2,3-difluoro-4- methoxy- phenyl)imidazo[1,2- a]pyrazin-8- yl]amino]-2-methyl- benzoyl]piperazine- 1-carboxamide hydrochloride | 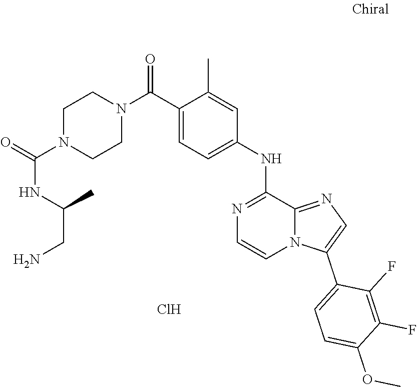 [SMILES: COc1ccc(-c2cnc3c(Nc4ccc(C(=O)N5CCN(C(=O)N[C@@H](C)CN)CC5)c(C)c4)nccn23)c(F)c1F ; Cl] | [M − H]−: 577.6 | Reference Example 208 and tert-butyl (S)-(2- aminopropyl)carbamate [CAS#121103-15-9]. Deprotection with 4M HCl in dioxane. |
| REF 315 | N-[(1R)-2-amino-1- methyl-ethyl]-4-[4- [[3-(2,3-difluoro-4- methoxy- phenyl)imidazo[1,2- a]pyrazin-8- yl]amino]-2-methyl- benzoyl]piperazine- 1-carboxamide hydrochloride | 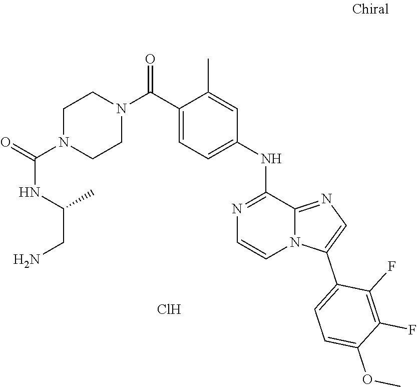 [SMILES: COc1ccc(-c2cnc3c(Nc4ccc(C(=O)N5CCN(C(=O)N[C@H](C)CN)CC5)c(C)c4)nccn23)c(F)c1F ; Cl] | 579.3 | Reference Example 208 and tert-butyl (R)-(2- aminopropyl)carbamate [CAS#333743-54-7]. Deprotection with 4M HCl in dioxane. |
| REF 316 | N-[(2S)-2- aminopropyl]-4-[4- [[3-(2,3-difluoro-4- methoxy- phenyl)imidazo[1,2- a]pyrazin-8- yl]amino]-2-methyl- benzoyl]piperazine- 1-carboxamide | 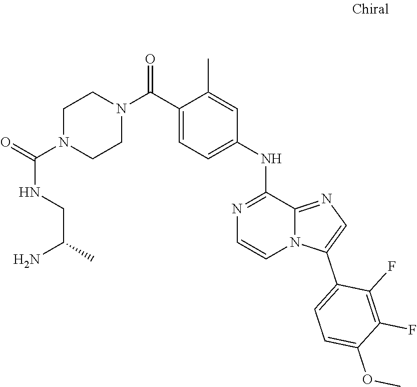 [SMILES: COc1ccc(-c2cnc3c(Nc4ccc(C(=O)N5CCN(C(=O)NC[C@H](C)N)CC5)c(C)c4)nccn23)c(F)c1F] | 579.3 | Reference Example 208 and tert-butyl (S)-(1- aminopropan-2- yl)carbamate [CAS#146552-71-8]. Deprotection with 4M HCl in dioxane. |
| REF 317 | N-[(2R)-2- aminopropyl]-4-[4- [[3-[4- (difluoromethoxy)phe- nyl]imidazo[1,2- a]pyrazin-8- yl]amino]-2-methyl- benzoyl]piperazine- 1-carboxamide hydrochloride | 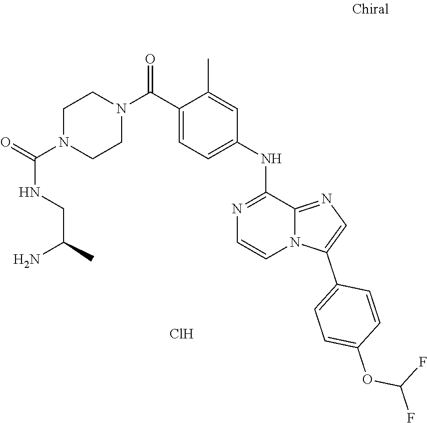 [SMILES: Cc1cc(Nc2nccn3c(-c4ccc(OC(F)F)cc4)cnc23)ccc1C(=O)N1CCN(C(=O)NC[C@@H](C)N)CC1 ; Cl] | [M − H]−: 577.3 | Reference Example 294 and tert-butyl (R)-(1- aminopropan-2- yl)carbamate hydrochloride [CAS#100927-10-4]. Deprotectino with 4M HCl in dioxane. |
| REF 318 | N-[(2S)-2- aminopropyl]-4-[4- [[3-[4- (difluoromethoxy)phe- nyl]imidazo[1,2- a]pyrazin-8- yl]amino]-2-methyl- benzoyl]piperazine- 1-carboxamide hydrochloride | 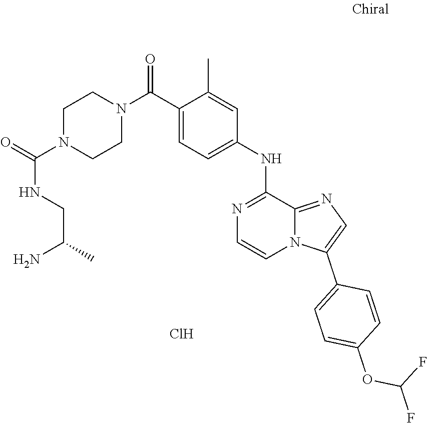 [SMILES: Cc1cc(Nc2nccn3c(-c4ccc(OC(F)F)cc4)cnc23)ccc1C(=O)N1CCN(C(=O)NC[C@H](C)N)CC1 ; Cl] | 579.3 | Reference Example 294 and tert-butyl (S)-(1- aminopropan-2- yl)carbamate hydrochloride [CAS#146552-71-8] |
| REF 319 | N-[(2R)-2- aminopropyl]-4-[4- [[3-(2,3-difluoro-4- methoxy- phenyl)imidazo[1,2- a]pyrazin-8- yl]amino]-2-methyl- benzoyl]piperazine- 1-carboxamide hydrochloride | 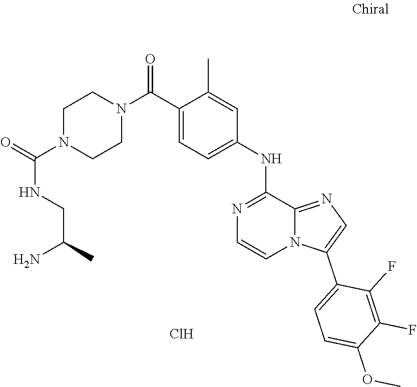 [SMILES: COc1ccc(-c2cnc3c(Nc4ccc(C(=O)N5CCN(C(=O)NC[C@@H](C)N)CC5)c(C)c4)nccn23)c(F)c1F ; Cl] | 579.3 | Reference Example 208 and tert-butyl (R)-(1- aminopropan-2- yl)carbamate hydrochloride [CAS#100927-10-4]. |
| REF 320 | N-(2-aminoethyl)-4- [4-[[3-(2-chloro-3- fluoro-4-methoxy- phenyl)imidazo[1,2- a]pyrazin-8- yl]amino]-2-methyl- benzoyl]piperazine- 1-carboxamide formate | 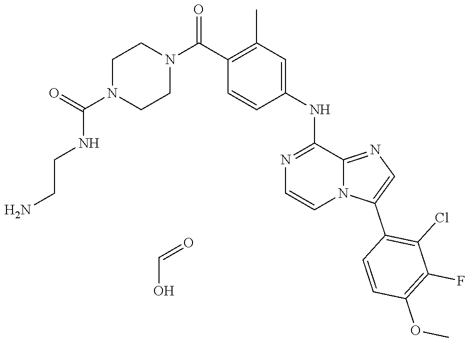 [SMILES: COc1ccc(-c2cnc3c(Nc4ccc(C(=O)N5CCN(C(=O)NCCN)CC5)c(C)c4)nccn23)c(Cl)c1F ; O=CO] | 581.2 | Intermediate 103 ([4- [[3-(2-chloro-3- fluoro-4-methoxy- phenyl)imidazo[1,2- a]pyrazin-8-yl]amino]- 2-methyl-phenyl]- piperazin-1-yl- methanone) and N- BOC-ethylenediamine. Purification: prep HPLC with formic acid |
| REF 321 | N-[(2R)-2- aminopropyl]-4-[4- [[3-(2-chloro-3- fluoro-4-methoxy- phenyl)imidazo[1,2- a]pyrazin-8- yl]amino]-2-methyl- benzoyl]piperazine- 1-carboxamide formate | 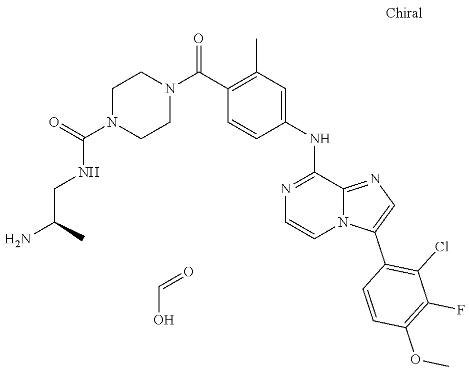 [SMILES: COc1ccc(-c2cnc3c(Nc4ccc(C(=O)N5CCN(C(=O)NC[C@@H](C)N)CC5)c(C)c4)nccn23)c(Cl)c1F ; O=CO] | 595.2 | Interemdiate 103 and tert-butyl (R)-(1- aminopropan-2- yl)carbamate hydrochloride [CAS#100927-10-4]. Purification: prep HPLC with formic acid |
| REF 322 | N-[(2S)-2- aminopropyl]-4-[4- [[3-(2-chloro-3- fluoro-4-methoxy- phenyl)imidazo[1,2- a]pyrazin-8- yl]amino]-2-methyl- benzoyl]piperazine- 1-carboxamide formate | 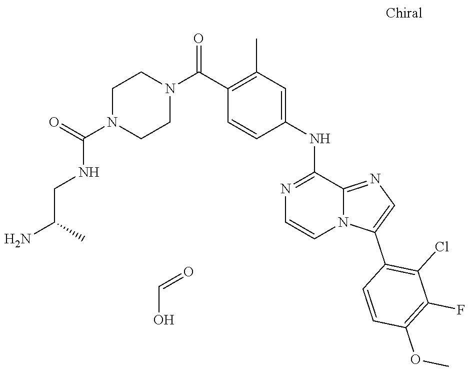 [SMILES: COc1ccc(-c2cnc3c(Nc4ccc(C(=O)N5CCN(C(=O)NC[C@H](C)N)CC5)c(C)c4)nccn23)c(Cl)c1F ; O=CO] | 595.2 | Intermediate 103 and tert-butyl (S)-(1- aminopropan-2- yl)carbamate hydrochloride [CAS#146552-71-8]. Purification: prep HPLC with formic acid |
| REF 323 | 2-[4-[8-[4-[4-[(2R)- 2- (aminomethyl)morpho- line-4- carbonyl]piperazine- 1-carbonyl]-3- methyl- anilino]imidazo[1,2- a]pyrazin-3-yl]-3- chloro-2-fluoro- phenoxy]acetonitrile formate | 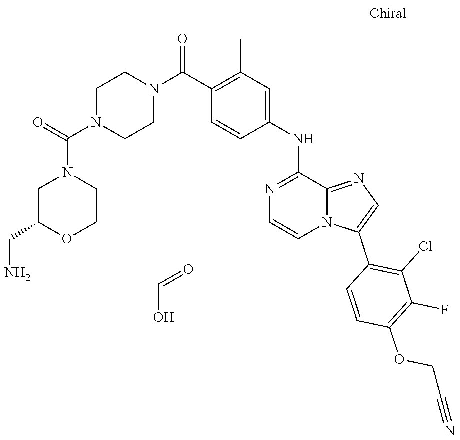 [SMILES: Cc1cc(Nc2nccn3c(-c4ccc(OCC#N)c(F)c4Cl)cnc23)ccc1C(=O)N1CCN(C(=O)N2CCO[C@H](CN)C2)CC1 ; O=CO] | 662.4 | Intermediate 111 and tert-butyl N-[[(2S)- morpholin-2- yl]methyl]carbamate. Deprotection in DCM:TFA 10:2, 20° C. Purification: prep HPLC with formic acid |
| REF 324 | 2-[4-[8-[4-[4-[(2S)- 2- (aminomethyl)morpho- line-4- carbonyl]piperazine- 1-carbonyl]-3- methyl- anilino]imidazo[1,2- a]pyrazin-3-yl]-3- chloro-2-fluoro- phenoxy]acetonitrile formate | 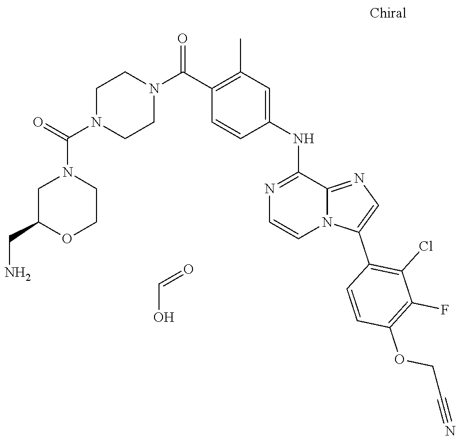 [SMILES: Cc1cc(Nc2nccn3c(-c4ccc(OCC#N)c(F)c4Cl)cnc23)ccc1C(=O)N1CCN(C(=O)N2CCO[C@@H](CN)C2)CC1 ; O=CO] | 662.4 | Intermediate 111 and tert-butyl N-[[(2R)- morpholin-2- yl]methyl]carbamate. Deprotection in DCM:TFA 10:2, 20° C. Purification: prep HPLC with formic acid |
| REF 325 | N-(2-aminoethyl)-4- [4-[[3-[2-chloro-4- (cyanomethoxy)-3- fluoro- phenyl]imidazo[1,2- a]pyrazin-8- yl]amino]-2-methyl- benzoyl]piperazine- 1-carboxamide formate | 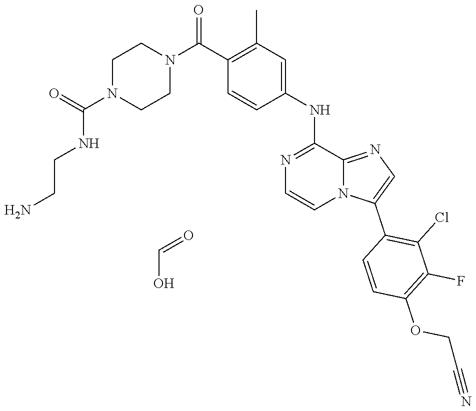 [SMILES: Cc1cc(Nc2nccn3c(-c4ccc(OCC#N)c(F)c4Cl)cnc23)ccc1C(=O)N1CCN(C(=O)NCCN)CC1 ; O=CO] | 606.2 | Intermediate 111 and N- BOC-ethylenediamine. Deprotection in DCM:TFA 10:2, 20° C. Purification: prep HPLC with formic acid |
| REF 326 | N-[(1R)-2-amino-1- methyl-ethyl]-4-[4- [[3-[2-chloro-4- (cyanomethoxy)-3- fluoro- phenyl]imidazo[1,2- a]pyrazin-8- yl]amino]-2-methyl- benzoyl]piperazine- 1-carboxamide formate | 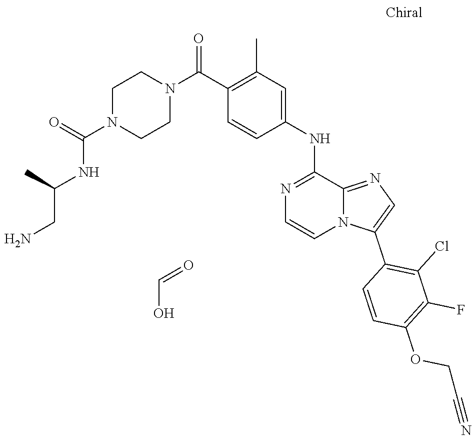 [SMILES: Cc1cc(Nc2nccn3c(-c4ccc(OCC#N)c(F)c4Cl)cnc23)ccc1C(=O)N1CCN(C(=O)N[C@H](C)CN)CC1 ; O=CO] | 620.3 | Intermediate 111 and tert-butyl (R)-(2- aminopropyl)carbamate [CAS#333743-54-7]. Deprotection in DCM:TFA 10:2, 20° C. Purification: prep HPLC with formic acid |
| REF 327 | N-[(1S)-2-amino-1- methyl-ethyl]-4-[4- [[3-[2-chloro-4- (cyanomethoxy)-3- fluoro- phenyl]imidazo[1,2- a]pyrazin-8- yl]amino]-2-methyl- benzoyl]piperazine- 1-carboxamide formate | 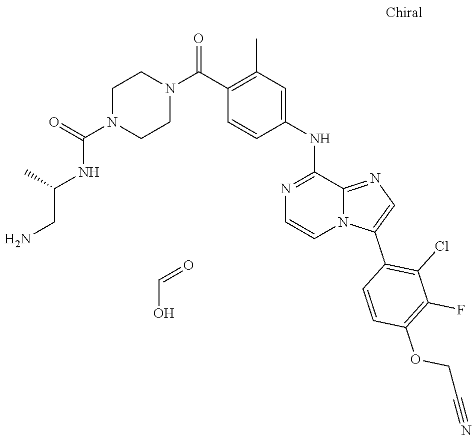 [SMILES: Cc1cc(Nc2nccn3c(-c4ccc(OCC#N)c(F)c4Cl)cnc23)ccc1C(=O)N1CCN(C(=O)N[C@@H](C)CN)CC1 ; O=CO] | 620.3 | Intermediate 111 and tert-butyl (R)-(2- aminopropyl)carbamate [CAS#333743-54-7]. Deprotection in DCM:TFA 10:2, 20° C. Purification: prep HPLC with formic acid |
| REF 328 | N-[(2R)-2- aminopropyl]-4-[4- [[3-[2-chloro-4- (cyanomethoxy)-3- fluoro- phenyl]imidazo[1,2- a]pyrazin-8- yl]amino]-2-methyl- benzoyl]piperazine- 1-carboxamide formate | 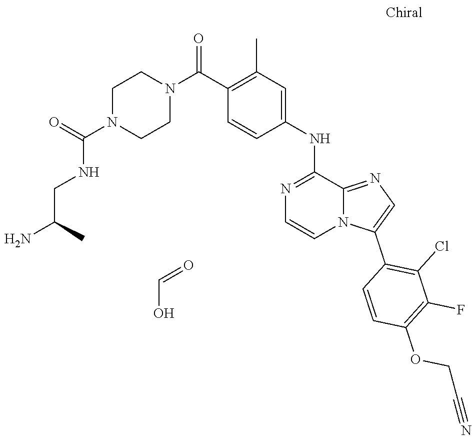 [SMILES: Cc1cc(Nc2nccn3c(-c4ccc(OCC#N)c(F)c4Cl)cnc23)ccc1C(=O)N1CCN(C(=O)NC[C@@H](C)N)CC1 ; O=CO] | 620.3 | Intermediate 111 and tert-butyl (R)-(1- aminopropan-2- yl)carbamate hydrochloride [CAS#100927-10-4]. Deprotection in DCM:TFA 10:2, 20° C. Purification: prep HPLC with formic acid |
| REF 329 | N-[(2S)-2- aminopropyl]-4-[4- [[3-[2-chloro-4- (cyanomethoxy)-3- fluoro- phenyl]imidazo[1,2- a]pyrazin-8- yl]amino]-2-methyl- benzoyl]piperazine- 1-carboxamide formate | 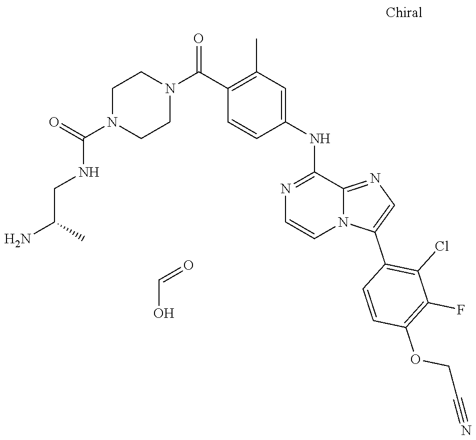 [SMILES: Cc1cc(Nc2nccn3c(-c4ccc(OCC#N)c(F)c4Cl)cnc23)ccc1C(=O)N1CCN(C(=O)NC[C@H](C)N)CC1 ; O=CO] | 620.4 | Intermediate 111 and tert-butyl (S)-(1- aminopropan-2- yl)cabamate hydrochloride [CAS#146552-71-8]. Deprotection in DCM:TFA 10:2, 20° C. Purification: prep HPLC with formic acid |
| REF 330 | N-[(1R)-2-amino-1- methyl-ethyl]-4-[4- [[3-(2-chloro-3- fluoro-4-methoxy- phenyl)imidazo[1,2- a]pyrazin-8- yl]amino]-2-methyl- benzoyl]piperazine- 1-carboxamide formate | 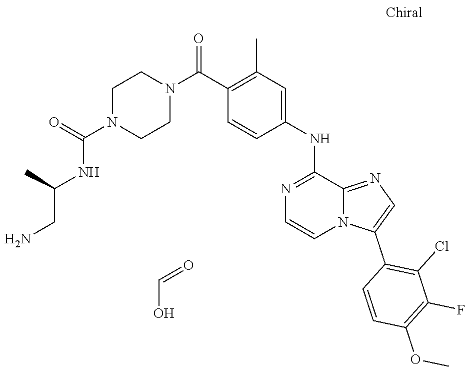 [SMILES: COc1ccc(-c2cnc3c(Nc4ccc(C(=O)N5CCN(C(=O)N[C@H](C)CN)CC5)c(C)c4)nccn23)c(Cl)c1F ; O=CO] | 595.1 | Intermediate 103 and tert-butyl (R)- (2- aminopropyl)carbamate [CAS#333743-54-7]. Purification: prep HPLC with formic acid |
| REF 331 | [4-[(2R)-2- (aminomethyl)morpho- line-4- carbonyl]piperazin- 1-yl]-[4-[[3-(2- chloro-3-fluoro-4- methoxy- phenyl)imidazo[1,2- a]pyrazin-8- yl]amino]-2-methyl- phenyl]methanone formate | 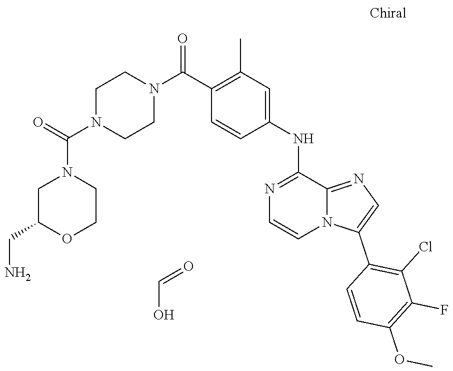 [SMILES: COc1ccc(-c2cnc3c(Nc4ccc(C(=O)N5CCN(C(=O)N6CCO[C@H](CN)C6)CC5)c(C)c4)nccn23)c(Cl)c1F ; O=CO] | 637.2 | Intermediate 103 and tert-butyl N- [[(2S)-morpholin-2- yl]methyl]carbamate Deprotection in DCM:TFA 2:1, rt. Purification: prep HPLC with formic acid |
| REF 332 | N-[3-(2- aminoethylcarbamoyl- amino)propyl]-4- [[3-(2,3-difluoro-4- methoxy- phenyl)imidazo[1,2- a]pyrazin-8- yl]amino]-2-methyl- benzamide | 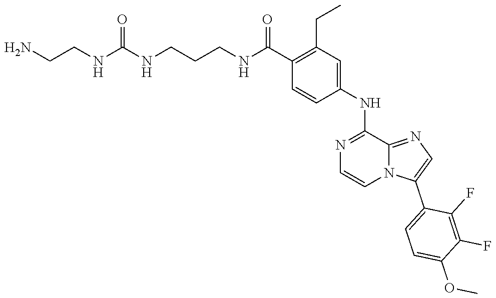 [SMILES: CCc1cc(Nc2nccn3c(-c4ccc(OC)c(F)c4F)cnc23)ccc1C(=O)NCCCNC(=O)NCCN] | 567.3 | Reference Example 297 and N-BOC- ethylenediamine |
| REF 333 | N-[2-(azetidin-1- yl)ethyl]-4-[4-[[3-[4- (difluoromethoxy)phe- nyl]imidazo[1,2- a]pyrazin-8- yl]amino]-2-methyl- benzoyl]piperazine- 1-carboxamide formate | 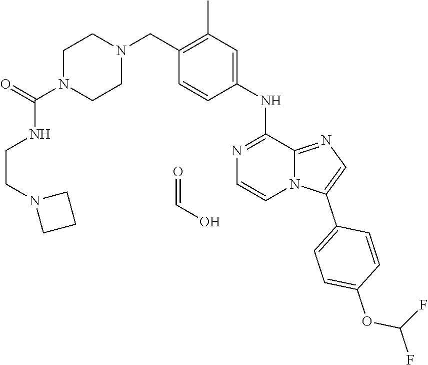 [SMILES: Cc1cc(Nc2nccn3c(-c4ccc(OC(F)F)cc4)cnc23)ccc1CN1CCN(C(=O)NCCN2CCC2)CC1 ; O=CO] | 605.3 | Reference Example 294 and 1- Azetidineethanamine |
| REF 334 | N-(azetidin-3-yl)-4- [4-[[3-[4- (difluoromethoxy)phe- nyl]imidazo[1,2- a]pyrazin-8- yl]amino]-2-methyl- benzoyl]piperazine- 1-carboxamide trifluoroacetate | 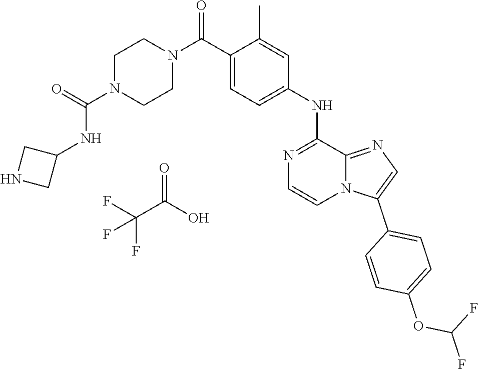 [SMILES: Cc1cc(Nc2nccn3c(-c4ccc(OC(F)F)cc4)cnc23)ccc1C(=O)N1CCN(C(=O)NC2CNC2)CC1 ; O=C(O)C(F)(F)F] | 577.3 | Reference Example 294 and 1-BOC-3- (amino)azetidine Deprotection: 2 h at rt in a 10/1 DCM/TFA mixture. |
| REF 335 | 4-[4-[[3-[4- (difluoromethoxy)phe- nyl]imidazo[1,2- a]pyrazin-8- yl]amino]-2-methyl- benzoyl]-N-[rac- (3R,4R)-4- hydroxypyrrolidin-3- yl]piperazine-1- carboxamide hydrochloride | 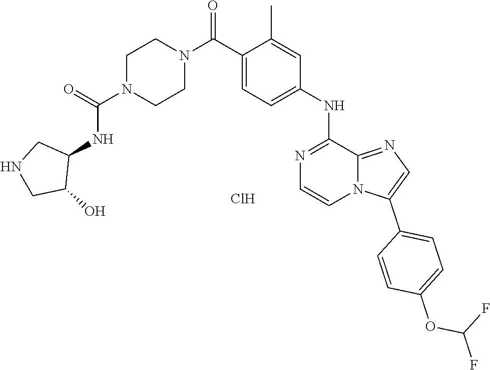 [SMILES: Cc1cc(Nc2nccn3c(-c4ccc(OC(F)F)cc4)cnc23)ccc1C(=O)N1CCN(C(=O)N[C@@H]2CNC[C@H]2O)CC1 ; Cl] | 607.3 | Reference Example 294 and rac-tert-butyl (3R,4R)-3-amino-4- hydroxypyrrolidine-1- carboxylate hydrochloride [CAS#148214-90-8] |
| REF 336 | 4-[4-[[3-[4- (difluoromethoxy)phe- nyl]imidazo[1,2- a]pyrazin-8- yl]amino]-2-methyl- benzoyl]-N-[rac- (3R,4R)-4- methoxypyrrolidin- 3-yl]piperazine-1- carboxamide hydrochloride | 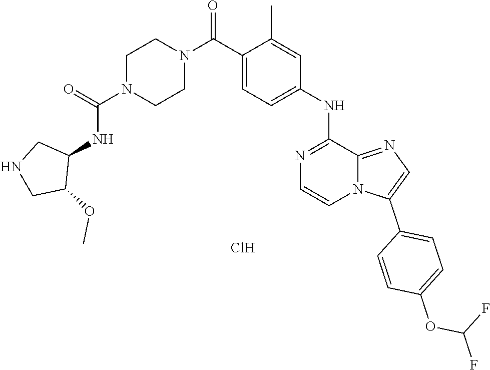 [SMILES: CO[C@@H]1CNC[C@H]1NC(=O)N1CCN(C(=O)c2ccc(Nc3nccn4c(-c5ccc(OC(F)F)cc5)cnc34)cc2C)CC1 ; Cl] | 621.3 | Reference Example 294 and rac-tert-butyl (3R,4R)-3-amino-4- methoxypyrrolidine-1- carboxylate [CAS#429673-79-0] |
| REF 337 | N-[3-(3-amino- propylcarbamoyl- amino)propyl]-4- [[3-(2,3-difluoro-4- methoxy- phenyl)imidazo[1,2- a]pyrazin-8- yl]amino]-2-ethyl- benzamide | 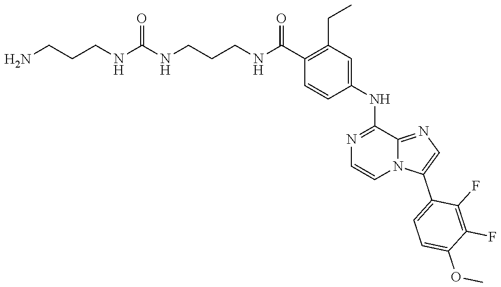 [SMILES: CCc1cc(Nc2nccn3c(-c4ccc(OC)c(F)c4F)cnc23)ccc1C(=O)NCCCNC(=O)NCCCN] | 581.2 | Reference Example 297 and N-BOC-1,3- diaminopropane |
| REF 338 | (R)-(4-(4-((3-(2,3- difluoro-4- methoxyphenyl)imida- zo[1,2-a]pyrazin-8- yl)amino)-2- methylbenzoyl)piper- azin-1-yl)(3- (hydroxymethyl)piper- azin-1- yl)methanone hydrochloride | 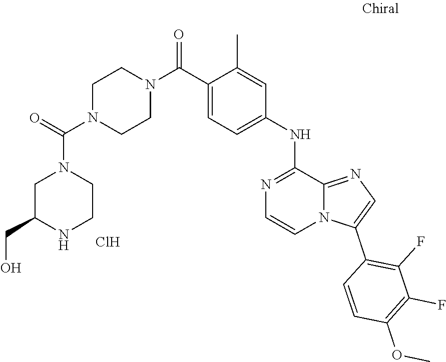 [SMILES: COc1ccc(-c2cnc3c(Nc4ccc(C(=O)N5CCN(C(=O)N6CCN[C@@H](CO)C6)CC5)c(C)c4)nccn23)c(F)c1F ; Cl] | 621.4 | From Reference Example 208 and tert- butyl (R)-2-(hydroxy- methyl)piperazine- 1-carboxylate |

### Example REF 339

### [4-[[3-[4-(difluoromethoxy)phenyl]imidazo[1,2-a]pyrazin-8-yl]amino]-2-methyl-phenyl]-[4-(piperidine-4-carbonyl)piperazin-1-yl]methanone Hydrochloride

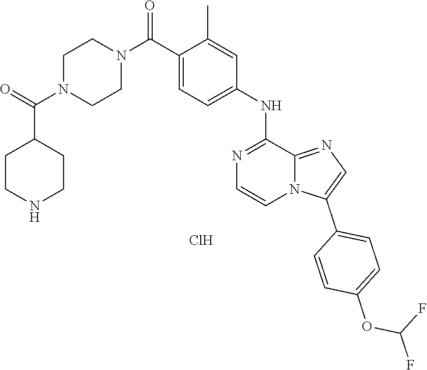 [SMILES: Cc1cc(Nc2nccn3c(-c4ccc(OC(F)F)cc4)cnc23)ccc1C(=O)N1CCN(C(=O)C2CCNCC2)CC1 ; Cl]

### Step 1) tert-butyl 4-[4-[4-[[3-[4-(difluoromethoxy)phenyl]imidazo[1,2-a]pyrazin-8-yl]amino]-2-methyl-benzoyl]piperazine-1-carbonyl]piperidine-1-carboxylate

In a 25 mL vial, (4-((3-(4-(difluoromethoxy)phenyl)imidazo[1,2-a]pyrazin-8-yl)amino)-2-methylphenyl)(piperazin-1-yl)methanone hydrochloride; Reference Example 294 (250 mg, 243 μmol, Eq: 1) and DIPEA (157 mg, 212 μL, 1.21 mmol, Eq: 5.0) were combined with DMF (5 mL) to give a light yellow suspension, that was stirred until most solids were dissolved. Then 1-(tert-butoxycarbonyl)piperidine-4-carboxylic acid (83.5 mg, 364 μmol, Eq: 1.5) and HATU (185 mg, 485 μmol, Eq: 2.0) were added. The walls of the tube were washed down with some DMF and the reaction mixture was stirred at RT for 2 h. The reaction mixture was poured into 10 mL ethyl acetate and extracted once with 0.1 M aq. NaOH. The organic phase was washed with brine, dried with magnesium sulfate monohydrate, filtered and the resulting solution was concentrated in vacuo. The crude material was purified by flash chromatography (silica gel, 25 g, 0% to 75% DCM/MeOH/aq. 25% NH₄OH (95/5/1)) leading to 101 mg off-white solid. MS: [M+H]+; 690.4

### Step 2) Reference Example 339

### [4-[[3-[4-(difluoromethoxy)phenyl]imidazo[1,2-a]pyrazin-8-yl]amino]-2-methyl-phenyl]-[4-(piperidine-4-carbonyl)piperazin-1-yl]methanone Hydrochloride

In a 50 mL round-bottomed flask, tert-butyl 4-(4-(4-((3-(4-(difluoromethoxy)phenyl)imidazo[1,2-a]pyrazin-8-yl)amino)-2-methylbenzoyl)piperazine-1-carbonyl)piperidine-1-carboxylate (Step 1) (100 mg, 145 μmol, Eq: 1) was combined with dioxane (1 mL) to give a colorless solution. Hydrogen chloride (4M in dioxane) (181 μl, 725 μmol, Eq: 5) was added. Stirring was continued and hydrogen chloride (4M in dioxane) (181 μl, 725 μmol, Eq: 5) was added again. The reaction mixture was stirred overnight. The reaction mixture was diluted with anhydrous ether, stirred and then filtered. The filter cake was washed with ether several times and dried in HV leading to the target compound as an off-white solid (93 mg, yield: 93%). MS (ISN): [M−H]⁻; 588.5

The following examples were prepared in analogy to Reference Example 339 (Step 2 only required if protecting group needs removal):

| | | | MS | |
|---|---|---|---|---|
| | | | ESI | |
| Ex. | Name | Structure | [M + H]⁺ | Starting Material |
| REF 340 | [4-[[3-(3-fluoro-4- methoxy- phenyl)imidazo[1,2- a]pyrazin-8- yl]amino]-2-methyl- phenyl]-[4- (pyrrolidine-2- carbonyl)piperazin-1- yl]methanone | 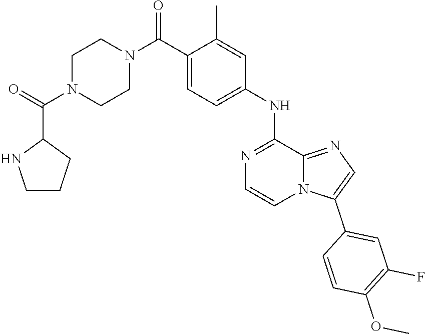 [SMILES: COc1ccc(-c2cnc3c(Nc4ccc(C(=O)N5CCN(C(=O)C6CCCN6)CC5)c(C)c4)nccn23)cc1F] | [M − H]−; 556.6 | Reference Example 295 and 1-(tert- butoxycarbonyl)pyrrolidine- 2-carboxylic acid. Deprotection: 45 min at rt. Purified by flash chromatography (silica gel, 0% to 100% DCM/MeOH/NH4OH (90/10/1)) |
| REF 341 | [4-[(2S)-azetidine-2- carbonyl]piperazin-1- yl]-[4-[[3-(3-fluoro-4- methoxy- phenyl)imidazo[1,2- a]pyrazin-8- yl]amino]-2-methyl- phenyl]methanone | 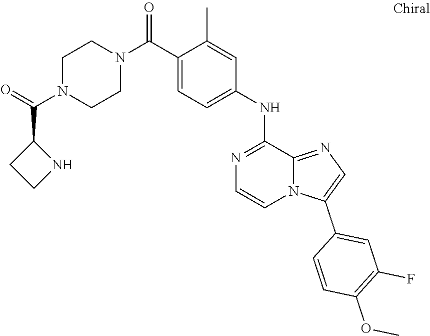 [SMILES: COc1ccc(-c2cnc3c(Nc4ccc(C(=O)N5CCN(C(=O)[C@@H]6CCN6)CC5)c(C)c4)nccn23)cc1F] | [M − H]−; 542.6 | Reference Example 295 and (S)-1-(tert- butoxycarbonyl)azetidine- 2-carboxylic acid. Deprotection: 45 min at rt. Purified by flash chromatography (silica gel, 0% to 100% DCM/MeOH/NH4OH (90/10/1)) |
| REF 342 | [4-[(2S)-azetidine-2- carbonyl]piperazin-1- yl]-[4-[[3-[4- (difluoromethoxy) phenyl]imidazo[1,2- a]pyrazin-8- yl]amino]-2-methyl- phenyl]methanone hydrochloride | 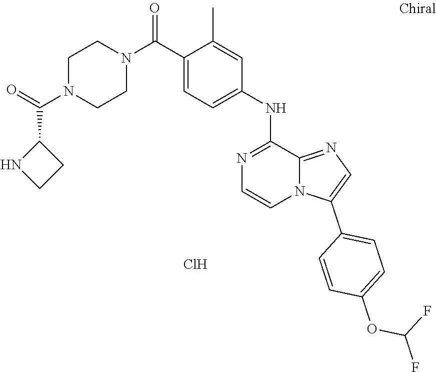 [SMILES: Cc1cc(Nc2nccn3c(-c4ccc(OC(F)F)cc4)cnc23)ccc1C(=O)N1CCN(C(=O)[C@@H]2CCN2)CC1 ; Cl] | [M − H]−; 560.2 | Reference Example 294 and (S)-1-(tert- butoxycarbonyl)azetidine- 2-carboxylic acid. |
| REF 343 | [4-[[3-[4- (difluoromethoxy) phenyl]imidazo[1,2- a]pyrazin-8- yl]amino]-2-methyl- phenyl]-[4- (pyrrolidine-2- carbonyl)piperazin-1- yl]methanone hydrochloride | 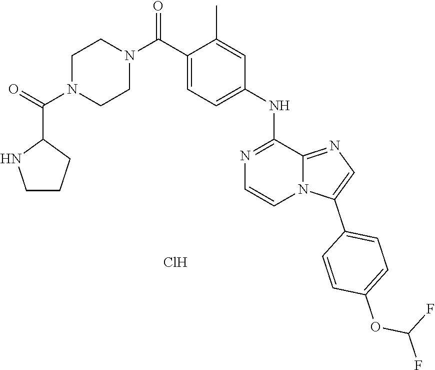 [SMILES: Cc1cc(Nc2nccn3c(-c4ccc(OC(F)F)cc4)cnc23)ccc1C(=O)N1CCN(C(=O)C2CCCN2)CC1 ; Cl] | [M − H]−; 556.6 | Reference Example 294 and 1-(tert- butoxycarbonyl)pyrrolidine- 2-carboxylic acid. |
| REF 344 | [4-[[3-(3-fluoro-4- methoxy- phenyl)imidazo[1,2- a]pyrazin-8- yl]amino]-2-methyl- phenyl]-[4-[(2S)- pyrrolidine-2- carbonyl]piperazin-1- yl]methanone hydrochloride | 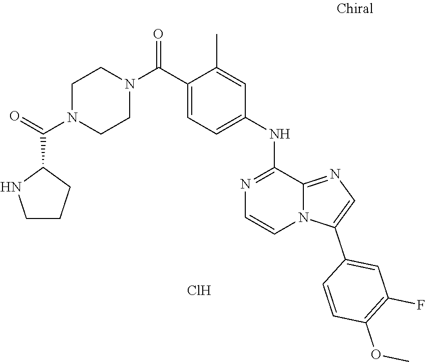 [SMILES: COc1ccc(-c2cnc3c(Nc4ccc(C(=O)N5CCN(C(=O)[C@@H]6CCCN6)CC5)c(C)c4)nccn23)cc1F ; Cl] | [M − H]−; 556.4 | Reference Example 295 and (S)-1-(tert- butoxycarbonyl) pyrrolidine- 2-carboxylic acid. |
| REF 345 | [4-[[3-(3-fluoro-4- methoxy- phenyl)imidazo[1,2- a]pyrazin-8- yl]amino]-2-methyl- phenyl]-[4-[(2R)- pyrrolidine-2- carbonyl]piperazin-1- yl]methanone hydrochloride | 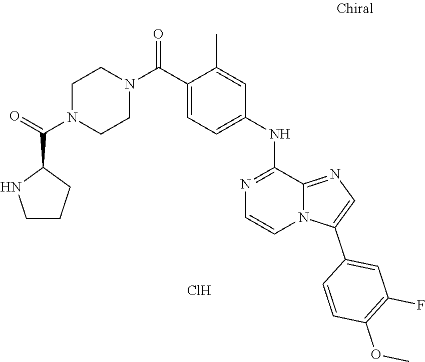 [SMILES: COc1ccc(-c2cnc3c(Nc4ccc(C(=O)N5CCN(C(=O)[C@H]6CCCN6)CC5)c(C)c4)nccn23)cc1F ; Cl] | [M − H]−; 556.4 | Reference Example 295 and (R)-1-(tert- butoxycarbonyl)pyrrolidine- 2-carboxylic acid. |
| REF 346 | [4-[[3-[4- (difluoromethoxy) phenyl]imidazo[1,2- a]pyrazin-8- yl]amino]-2-methyl- phenyl]-[4-[(2S,4R)- 4-hydroxypyrrolidine- 2-carbonyl]piperazin- 1-yl]methanone hydrochloride | 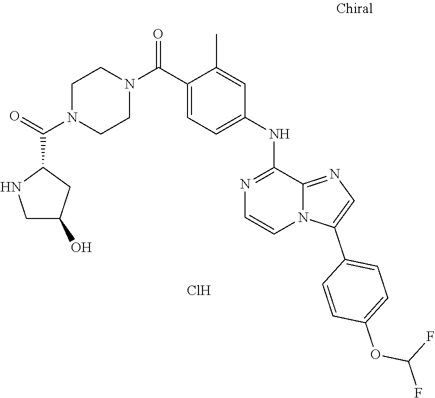 [SMILES: Cc1cc(Nc2nccn3c(-c4ccc(OC(F)F)cc4)cnc23)ccc1C(=O)N1CCN(C(=O)[C@@H]2C[C@@H](O)CN2)CC1 ; Cl] | [M − H]−; 590.4 | Reference Example 294 and (2S,4R)-1- (tert-butoxycarbonyl)- 4-hydroxypyrrolidine- 2-carboxylic acid. |
| REF 347 | [4-[[3-[4- (difluoromethoxy) phenyl]imidazo[1,2- a]pyrazin-8- yl]amino]-2-methyl- phenyl]-[4-[2- (hydroxymethyl) piperazine-1-carbonyl]-1- piperidyl]methanone hydrochloride | 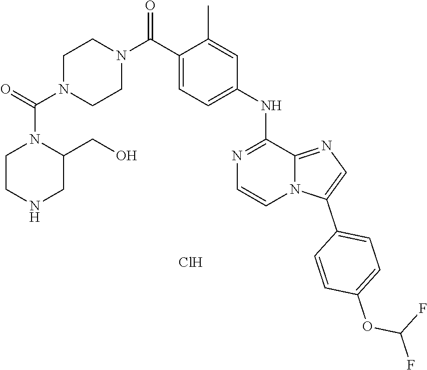 [SMILES: Cc1cc(Nc2nccn3c(-c4ccc(OC(F)F)cc4)cnc23)ccc1C(=O)N1CCN(C(=O)N2CCNCC2CO)CC1 ; Cl] | [M + H]+; 620.2 | Reference Example 294 and tert-butyl 3- (hydroxymethyl)piperazine- 1-carboxylate |
| REF 348 | [4-[[3-[4- (difluoromethoxy) phenyl]imidazo[1,2- a]pyrazin-8- yl]amino]-2-methyl- phenyl]-[4-[(2S,4S)-4- hydroxypyrrolidine-2- carbonyl]piperazin-1- yl]methanone hydrochloride hydrochloride | 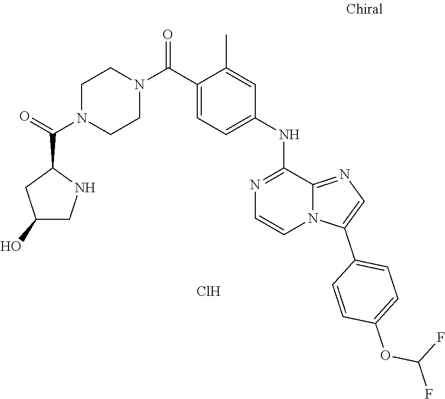 [SMILES: Cc1cc(Nc2nccn3c(-c4ccc(OC(F)F)cc4)cnc23)ccc1C(=O)N1CCN(C(=O)[C@@H]2C[C@H](O)CN2)CC1 ; Cl] | [M − H]−; 590.5 | Reference Example 294 and (2S,4S)-1- (tert-butoxycarbonyl)- 4-hydroxypyrrolidine- 2-carboxylic acid. Deprotection: 5eq HCl in dioxane (4M) in diethylether/MeOH 5/2 at rt overnight. |
| REF 349 | [4-[[3-[4- (difluoromethoxy) phenyl]imidazo[1,2- a]pyrazin-8- yl]amino]-2-methyl- phenyl]-[4-[3- (hydroxymethyl) piperazine-1-carbonyl]-1- piperidyl]methanone hydrochloride | 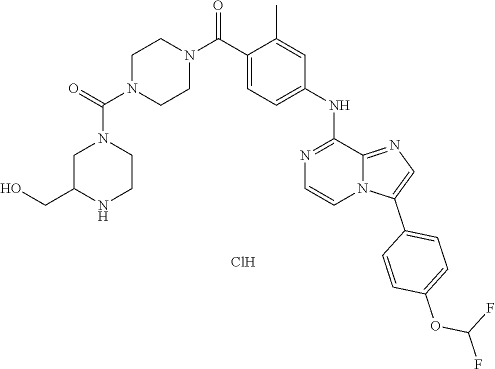 [SMILES: Cc1cc(Nc2nccn3c(-c4ccc(OC(F)F)cc4)cnc23)ccc1C(=O)N1CCN(C(=O)N2CCNC(CO)C2)CC1 ; Cl] | 620.5 (M + H) | Reference Example 1 and tert-butyl 2- (hydroxymethyl) piperazine-1-carboxylate. Deprotection with 10 eq HCl 4M in dioxane, 45 min at rt. |
| REF 350 | [4-[[3-(2,3-difluoro-4- methoxy- phenyl)imidazo[1,2- a]pyrazin-8- yl]amino]-2-methyl- phenyl]-[4-[(2S,4R)- 4-hydroxypyrrolidine- 2-carbonyl]piperazin- 1-yl]methanone hydrochloride | 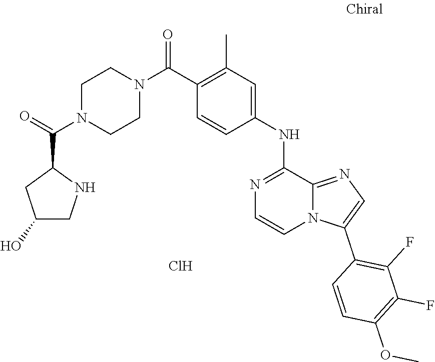 [SMILES: COc1ccc(-c2cnc3c(Nc4ccc(C(=O)N5CCN(C(=O)[C@@H]6C[C@@H](O)CN6)CC5)c(C)c4)nccn23)c(F)c1F ; Cl] | [M − H]−; 590.5 | Reference Example 208 and (2S,4R)-1- (tert-butoxycarbonyl)- 4-hydroxypyrrolidine- 2-carboxylic acid [CAS#13726-69-7] |
| REF 351 | [4-[[3-(2,3-difluoro-4- methoxy- phenyl)imidazo[1,2- a]pyrazin-8- yl]amino]-2-ethyl- phenyl]-[4-[(2S,4R)- 4-hydroxypyrrolidine- 2-carbonyl]piperazin- 1-yl]methanone hydrochloride | 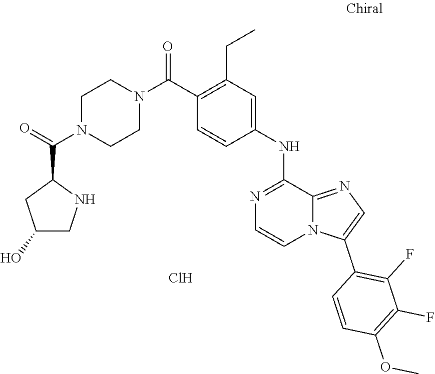 [SMILES: CCc1cc(Nc2nccn3c(-c4ccc(OC)c(F)c4F)cnc23)ccc1C(=O)N1CCN(C(=O)[C@@H]2C[C@@H](O)CN2)CC1 ; Cl] | 606.4 | Intermediate 110 and (2S,4R)-1-(tert- butoxycarbonyl)-4- hydroxypyrrolidine-2- carboxylic acid [CAS#13726-69-7]. Purification: prep HPLC with HCl |
| REF 352 | [4-[[3-(3-fluoro-4- methoxy- phenyl)imidazo[1,2- a]pyrazin-8- yl]amino]-2-methyl- phenyl]-[4-[(3S)- pyrrolidine-3- carbonyl]piperazin-1- yl]methanone hydrochloride | 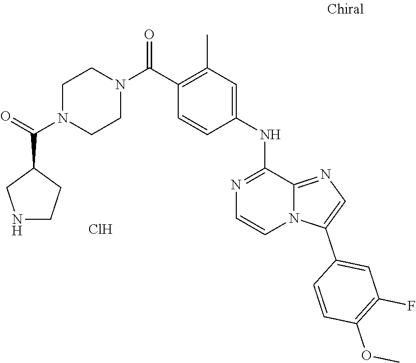 [SMILES: COc1ccc(-c2cnc3c(Nc4ccc(C(=O)N5CCN(C(=O)[C@H]6CCNC6)CC5)c(C)c4)nccn23)cc1F ; Cl] | 558.5 | Reference Example 295 and (3S)-1-(tert- Butoxycarbonyl)-3- pyrrolidinecarboxylic acid |
| REF 353 | [4-(azetidine-3- carbonyl)piperazin-1- yl]-[4-[[3-(3-fluoro-4- methoxy- phenyl)imidazo[1,2- a]pyrazin-8- yl]amino]-2-methyl- phenyl]methanone hydrochloride | 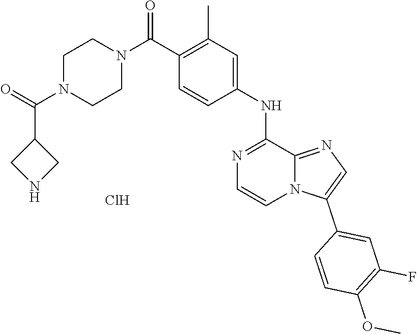 [SMILES: COc1ccc(-c2cnc3c(Nc4ccc(C(=O)N5CCN(C(=O)C6CNC6)CC5)c(C)c4)nccn23)cc1F ; Cl] | 544.5 | Reference Example 295 and 1-Boc- Azetidine-3-carboxylic acid |
| REF 354 | 2-[2,3-difluoro-4-[8- [4-[4-[(3R,4R)-3- hydroxypiperidine-4- carbonyl]piperazine- 1-carbonyl]-3-methyl- anilino]imidazo[1,2- a]pyrazin-3- yl]phenoxy]acetonitrile formate | 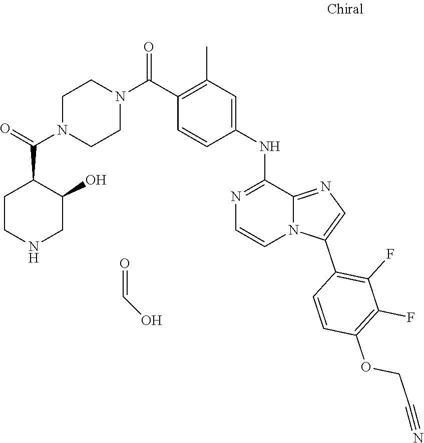 [SMILES: Cc1cc(Nc2nccn3c(-c4ccc(OCC#N)c(F)c4F)cnc23)ccc1C(=O)N1CCN(C(=O)[C@@H]2CCNC[C@@H]2O)CC1 ; O=CO] | 631.1 | Intermediate 109 and (3R,4R)-1-tert- butoxycarbonyl-3- hydroxy-piperidine-4- carboxylic acid [CAS#1932301-36-4]. Deprotection: 2h at rt in a 80/20 DCM/TFA mixture (95 eq TFA), then neutralized with sat. Na₂CO₃ to pH8. Purification: prep HPLC with formic acid |
| REF 355 | 2-[2,3-difluoro-4-[8- [4-[4-[(3S,4S)-3- hydroxypiperidine-4- carbonyl]piperazine- 1-carbonyl]-3-methyl- anilino]imidazo[1,2- a]pyrazin-3- yl]phenoxy]acetonitrile formate | 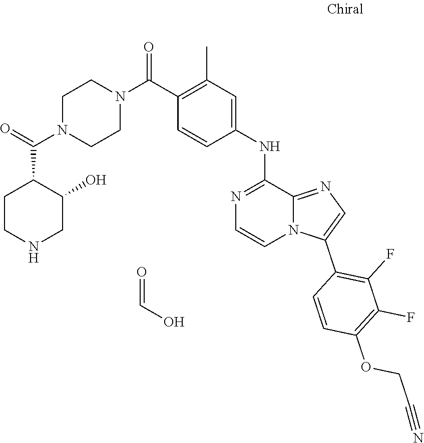 [SMILES: Cc1cc(Nc2nccn3c(-c4ccc(OCC#N)c(F)c4F)cnc23)ccc1C(=O)N1CCN(C(=O)[C@H]2CCNC[C@H]2O)CC1 ; O=CO] | 631.1 | Intermediate 109 and (3S,4S)-1-tert- butoxycarbonyl-3- hydroxy-piperidine-4- carboxylic acid, obtained by saponification of 1,4- Piperidinedicarboxylic acid, 3-hydroxy-, 1-(1, 1-dimethylethyl) 4- methyl ester, (3S,4S)- [CAS#2166250-53-7] Deprotection: 2h at rt in a 80/20 DCM/TFA mixture (95 eq TFA), then neutralized with sat. Na₂CO₃ to pH 8. Purification: prep. HPLC in presence of formic acid |
| REF 356 | 2-[2,3-difluoro-4-[8- [4-[4-[(3S,4R)-3- hydroxypiperidine-4- carbonyl]piperazine- 1-carbonyl]-3-methyl- anilino]imidazo[1,2- a]pyrazin-3- yl]phenoxy]acetonitrile- formate | 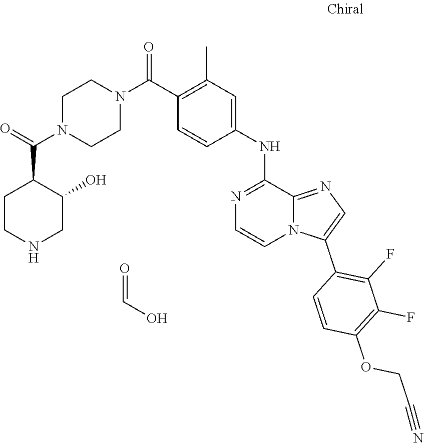 [SMILES: Cc1cc(Nc2nccn3c(-c4ccc(OCC#N)c(F)c4F)cnc23)ccc1C(=O)N1CCN(C(=O)[C@@H]2CCNC[C@H]2O)CC1 ; O=CO] | 631.1 | Intermediate 109 and (3S,4R)-1-tert- butoxycarbonyl-3- hydroxy-piperidine-4- carboxylic acid obtained by saponification of 1,4- Piperidinedicarboxylic acid, 3-hydroxy-, 1-(1, 1-dimethylethyl) 4- ethyl ester, (3S,4R)- [CAS#1805790-50-4]. Deprotection: 2h at rt in a 80/20 DCM/TFA mixture (95 eq TFA), then neutralized with sat. Na2CO3 to pH8. Purification: prep HPLC with formic acid as additive |
| REF 357 | 2-[2,3-difluoro-4-[8- [4-[4-[(3R,4S)-3- hydroxypiperidine-4- carbonyl]piperazine- 1-carbonyl]-3-methyl- anilino]imidazo[1,2- a]pyrazin-3- yl]phenoxy]acetonitrile formate | 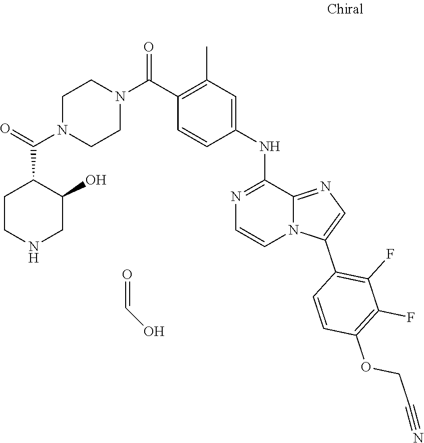 [SMILES: Cc1cc(Nc2nccn3c(-c4ccc(OCC#N)c(F)c4F)cnc23)ccc1C(=O)N1CCN(C(=O)[C@H]2CCNC[C@@H]2O)CC1 ; O=CO] | 631.1 | Intermediate 109 and 1, 4-Piperidinedicarboxylic acid, 3-hydroxy-, 1- (1,1-dimethylethyl) ester, (3R,4S)- [CAS# 1821775-81-8]. Deprotection: 2h at rt in a 80/20 DCM/TFA mixture (95 eq TFA), then neutralized with sat. Na2CO3 to pH8. Purification: prep HPLC with formic acid as additive |
| REF 358 | [4-[[3-(2,3-difluoro-4- methoxy- phenyl)imidazo[1,2- a]pyrazin-8- yl]amino]-2-methyl- phenyl]-[4-[(3R,4S)- 3-hydroxypiperidine- 4-carbonyl]piperazin- 1-yl]methanone hydrochloride | 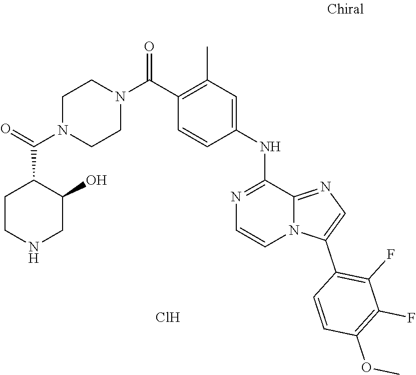 [SMILES: COc1ccc(-c2cnc3c(Nc4ccc(C(=O)N5CCN(C(=O)[C@H]6CCNC[C@@H]6O)CC5)c(C)c4)nccn23)c(F)c1F ; Cl] | 606.3 | Reference Example 208 and 1,4- Piperidinedicarboxylic acid, 3-hydroxy-, 1- (1,1-dimethylethyl) ester, (3R,4S)- [CAS# 1821775-81-8]. |
| REF 359 | [4-[[3-(2,3-difluoro-4- methoxy- phenyl)imidazo[1,2- a]pyrazin-8- yl]amino]-2-methyl- phenyl]-[4-[(3S,4R)- 3-hydroxypiperidine- 4-carbonyl]piperazin- 1-yl]methanone hydrochloride | 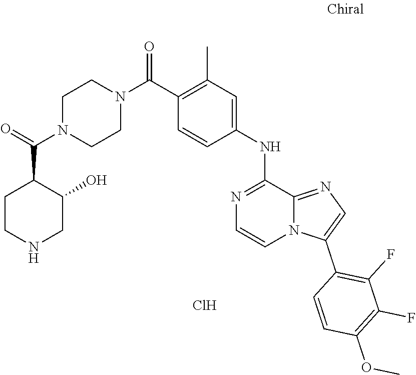 [SMILES: COc1ccc(-c2cnc3c(Nc4ccc(C(=O)N5CCN(C(=O)[C@@H]6CCNC[C@H]6O)CC5)c(C)c4)nccn23)c(F)c1F ; Cl] | 606.3 | Reference Example 208 and (3S,4R)-1-tert- butoxycarbonyl-3- hydroxy-piperidine-4- carboxylic acid obtained by saponification of 1,4- Piperidinedicarboxylic acid, 3-hydroxy-, 1- (1,1-dimethylethyl) 4- ethyl ester, (3S,4R)- [CAS# 1805790-50-4]. |
| REF 360 | 2-[4-[8-[3-ethyl-4-[4- (piperidine-4- carbonyl)piperazine- 1- carbonyl]anilino]imidazo [1,2-a]pyrazin-3- yl]-2,3-difluoro- phenoxy]acetonitrile formate | 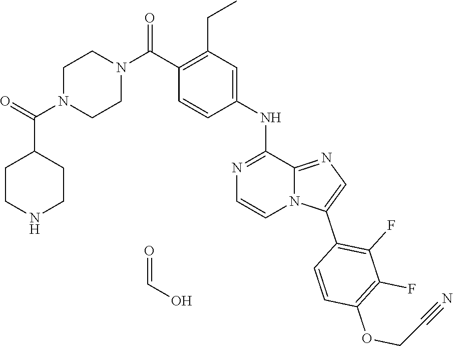 [SMILES: CCc1cc(Nc2nccn3c(-c4ccc(OCC#N)c(F)c4F)cnc23)ccc1C(=O)N1CCN(C(=O)C2CCNCC2)CC1 ; O=CO] | 629.1 | Intermediate 112 and 1- (tert- butoxycarbonyl)piperidine- 4-carboxylic acid. Deprotection: 0.5h at rt in a 80/20 DCM/TFA mixture (13 eq TFA), then neutralized with sat. Na2CO3 to pH8. Purification: prep HPLC with formic acid as additive |
| REF 361 | [4-[[3-(2,3-difluoro-4- methoxy- phenyl)imidazo[1,2- a]pyrazin-8- yl]amino]-2-methyl- phenyl]-[4-[rac- (3S,4S)-3- hydroxypiperidine-4- carbonyl]piperazin-1- yl]methanone | 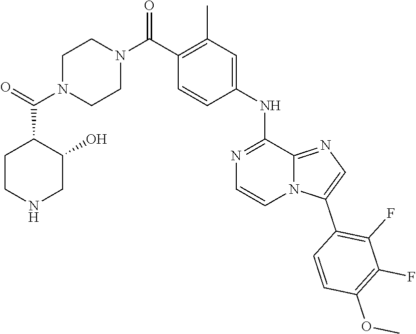 [SMILES: COc1ccc(-c2cnc3c(Nc4ccc(C(=O)N5CCN(C(=O)[C@H]6CCNC[C@H]6O)CC5)c(C)c4)nccn23)c(F)c1F] | 606.3 | Reference Example 208 and (3S,4S)-1-tert- butoxycarbonyl-3- hydroxy-piperidine-4- carboxylic acid, obtained by saponification of 1,4- Piperidinedicarboxylic acid, 3-hydroxy-, 1- (1,1-dimethylethyl) 4- methyl ester, (3S,4S)- [CAS# 2166250-53-7] Purification: prep HPLC |
| REF 362 | N-[2-[[(2S)-2-amino- 5-guanidino- pentanoyl]amino]ethyl]- 4-[[3-(2,3-difluoro- 4-methoxy- phenyl)imidazo[1,2- a]pyrazin-8- yl]amino]-2-ethyl- benzamide trifluoroacetate | 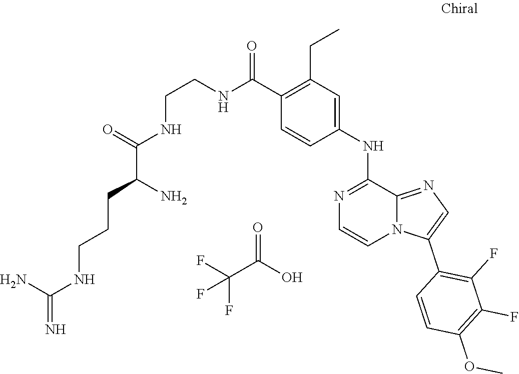 [SMILES: CCc1cc(Nc2nccn3c(-c4ccc(OC)c(F)c4F)cnc23)ccc1C(=O)NCCNC(=O)[C@@H](N)CCCNC(=N)N ; O=C(O)C(F)(F)F] | 623.3 | Reference Example 296 and FMOC-ARG- OH. Deprotection using 22 eq piperidine at 0- 20° C. for 12h. Purification by prep HPLC (TFA as additive). |
| 40 | 1-(4-(2-chloro-4-((3- (3-fluoro-4- methoxyphenyl)imidazo [1,2-a]pyrazin-8- yl)amino)benzoyl) piperazin-1-yl)-2- (methylamino)ethanone | 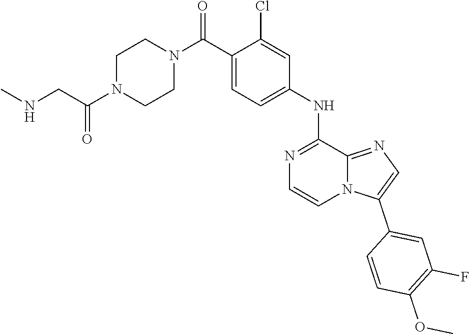 [SMILES: CNCC(=O)N1CCN(C(=O)c2ccc(Nc3nccn4c(-c5ccc(OC)c(F)c5)cnc34)cc2Cl)CC1] | 552.2 | Intermediate 123 and 2- ((tert- butoxycarbonyl)(methyl) amino)acetic acid |
| REF 363 | 2-[2,3-difluoro-4-[8- [3-methyl-4-[4-[rac- (3R,4R)-3- hydroxypiperidine-4- carbonyl]piperazine- 1- carbonyl]anilino]imidazo [1,2-a]pyrazin-3- yl]phenoxy]acetonitrile formate | 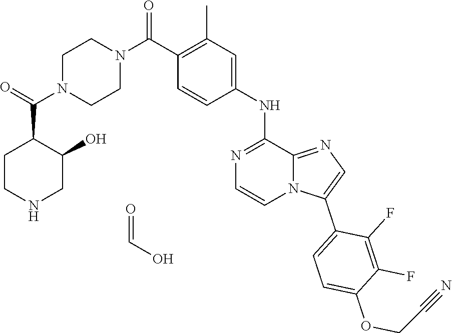 [SMILES: Cc1cc(Nc2nccn3c(-c4ccc(OCC#N)c(F)c4F)cnc23)ccc1C(=O)N1CCN(C(=O)[C@@H]2CCNC[C@@H]2O)CC1 ; O=CO] | 631.3 | Intermediate 109 and 1, 4-Piperidinedicarboxylic acid, 3-hydroxy-, 1- (1,1-dimethylethyl) ester, (3R,4R)-rel- [CAS# 1932301-36-4]. Deprotection: 1h at rt in a 80/20 DCM/TFA mixture (13 eq TFA), then neutralized with sat. Na2CO3 to pH8. Purification: prep HPLC with formic acid as additive |
| REF 364 | 2-[2,3-difluoro-4-[8- [3-methyl-4-[4- (piperidine-4- carbonyl)piperazine- 1- carbonyl]anilino]imidazo [1,2-a]pyrazin-3- yl]phenoxy]acetonitrile formate | 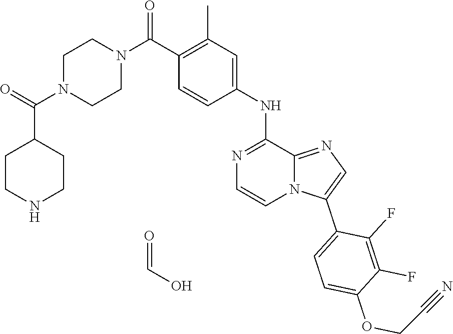 [SMILES: Cc1cc(Nc2nccn3c(-c4ccc(OCC#N)c(F)c4F)cnc23)ccc1C(=O)N1CCN(C(=O)C2CCNCC2)CC1 ; O=CO] | 615.3 | Intermediate 109 and N-BOC-isonipecotic acid. Deprotection: 1h at rt in a 80/20 DCM/TFA mixture (13 eq TFA), then neutralized with sat. Na2CO3 to pH8. Purification: prep. HPLC with formic acid as additive |
| REF 365 | 2-[2,3-difluoro-4-[8- [3-methyl-4-[4-[(3R)- pyrrolidine-3- carbonyl]piperazine- 1- carbonyl]anilino]imidazo [1,2-a]pyrazin-3- yl]phenoxy]acetonitrile formate | 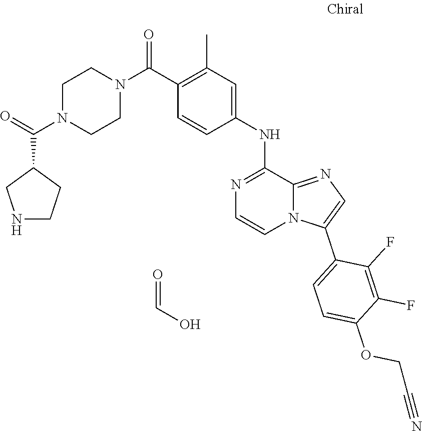 [SMILES: Cc1cc(Nc2nccn3c(-c4ccc(OCC#N)c(F)c4F)cnc23)ccc1C(=O)N1CCN(C(=O)[C@@H]2CCNC2)CC1 ; O=CO] | 601.3 | Intermediate 109 and (R)-1-BOC- pyrrolidine-3- carboxylic acid. Deprotection: 1h at rt in a 80/20 DCM/TFA mixture (60 eq TFA), then neutralized with sat. Na2CO3 to pH8. Purification: prep. HPLC with formic acid as additive |
| REF 366 | 2-[4-[8-[3-ethyl-4-[4- [rac-(3S,4S)-3- hydroxypiperidine-4- carbonyl]piperazine- 1- carbonyl]anilino]imidazo [1,2-a]pyrazin-3- yl]-2,3-difluoro- phenoxy]acetonitrile formate | 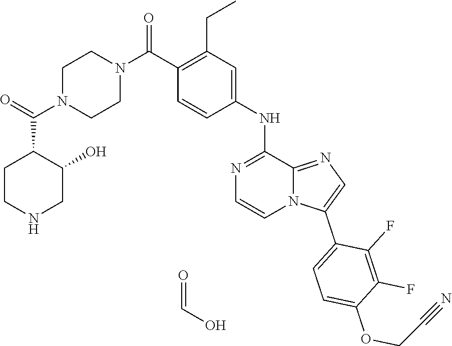 [SMILES: CCc1cc(Nc2nccn3c(-c4ccc(OCC#N)c(F)c4F)cnc23)ccc1C(=O)N1CCN(C(=O)[C@H]2CCNC[C@H]2O)CC1 ; O=CO] | 645.3 | Intermediate 112 and (3S,4S)-1-tert- butoxycarbonyl-3- hydroxy-piperidine-4- carboxylic acid, obtained by saponification of 1,4- Piperidinedicarboxylic acid, 3-hydroxy-, 1- (1,1-dimethylethyl) 4- methyl ester, (3S,4S)- [CAS# 2166250-53-7] Deprotection: 1h at rt in a 80/20 DCM/TFA mixture (95 eq TFA), then neutralized with sat. Na2CO3 to pH8. Purification: prep HPLC |
| REF 367 | 2-[2,3-difluoro-4-[8- [4-[4-[(2S,4R)-4- hydroxypyrrolidine-2- carbonyl]piperazine- 1-carbonyl]-3-methyl- anilino]imidazo[1,2- a]pyrazin-3- yl]phenoxy]acetonitrile formate | 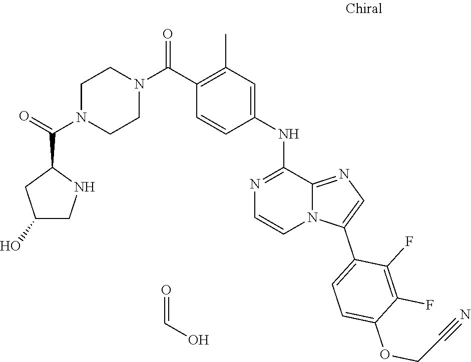 [SMILES: Cc1cc(Nc2nccn3c(-c4ccc(OCC#N)c(F)c4F)cnc23)ccc1C(=O)N1CCN(C(=O)[C@@H]2C[C@@H](O)CN2)CC1 ; O=CO] | 617.3 | Intermediate 109 and (2R,4S)-1-tert- butoxycarbonyl-4- hydroxy-pyrrolidine- 2-carboxylic acid. Deprotection: 1h at rt in a 80/20 DCM/TFA mixture (62 eq TFA), then neutralized with sat. Na2CO3 to pH8. Purification: prep HPLC |
| REF 368 | 2-[2,3-difluoro-4-[8- [3-methyl-4-[4-[(3S)- pyrrolidine-3- carbonyl]piperazine- 1- carbonyl]anilino]imidazo [1,2-a]pyrazin-3- yl]phenoxy]acetonitrile; formic acid | 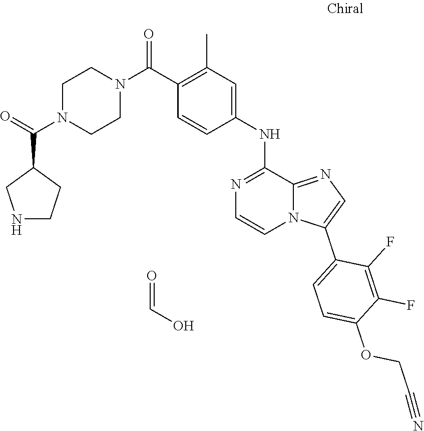 [SMILES: Cc1cc(Nc2nccn3c(-c4ccc(OCC#N)c(F)c4F)cnc23)ccc1C(=O)N1CCN(C(=O)[C@H]2CCNC2)CC1 ; O=CO] | 601.1 | Intermediate 109 and (S)-1-BOC-pyrrolidine- 3-carboxylic acid. Deprotection: 1h at rt in a 100/10 DCM/TFA mixture (160 eq TFA), Purification: prep HPLC |
| REF 369 | (R)-(4-(4-((3-(2,3- difluoro-4- methoxyphenyl)imidazo [1,2-a]pyrazin-8- yl)amino)-2- methylbenzoyl)piperazin- 1-yl)(pyrrolidin-3- yl)methanone hydrochloride | 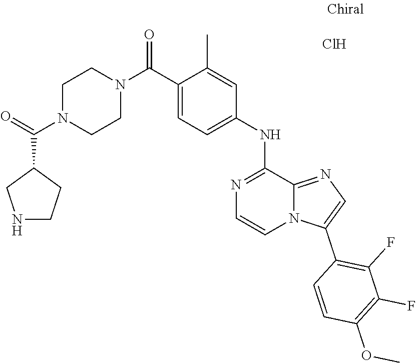 [SMILES: COc1ccc(-c2cnc3c(Nc4ccc(C(=O)N5CCN(C(=O)[C@@H]6CCNC6)CC5)c(C)c4)nccn23)c(F)c1F ; Cl] | 576.5 | Reference Example 208 and (R)-1-BOC- pyrrolidine-3- carboxylic acid |
| REF 370 | [4-(azetidine-3- carbonyl)piperazin-1- yl]-[4-[[3-(2,3- difluoro-4-methoxy- phenyl)imidazo[1,2- a]pyrazin-8- yl]amino]-2-methyl- phenyl]methanone; hydrochloride | 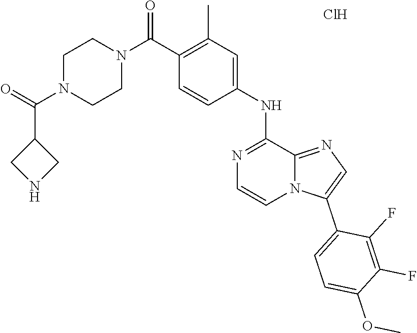 [SMILES: COc1ccc(-c2cnc3c(Nc4ccc(C(=O)N5CCN(C(=O)C6CNC6)CC5)c(C)c4)nccn23)c(F)c1F ; Cl] | 562.7 | Reference Example 208 and 1-(tert- butoxycarbonyl)azetidine- 3-carboxylic acid |
| REF 371 | [4-[[3-(2,3-difluoro-4- methoxy- phenyl)imidazo[1,2- a]pyrazin-8- yl]amino]-2-methyl- phenyl]-[4-(4- hydroxypiperidine-4- carbonyl)piperazin-1- yl]methanone hydrochloride | 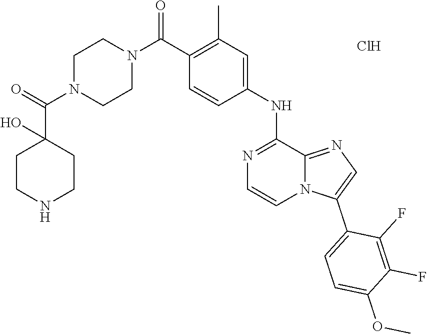 [SMILES: COc1ccc(-c2cnc3c(Nc4ccc(C(=O)N5CCN(C(=O)C6(O)CCNCC6)CC5)c(C)c4)nccn23)c(F)c1F ; Cl] | 606.5 | Reference Example 208 and 1-(tert- butoxycarbonyl)-4- hydroxypiperidine-4- carboxylic acid |
| REF 372 | [4-(3- azabicyclo[3.2.1]octane- 8- carbonyl)piperazin-1- yl]-[4-[[3-(2,3- difluoro-4-methoxy- phenyl)imidazo[1,2- a]pyrazin-8- yl]amino]-2-methyl- phenyl]methanone hydrochloride | 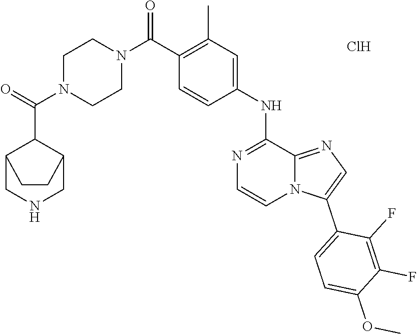 [SMILES: COc1ccc(-c2cnc3c(Nc4ccc(C(=O)N5CCN(C(=O)C6C7CCC6CNC7)CC5)c(C)c4)nccn23)c(F)c1F ; Cl] | 616.4 | Reference Example 208 and 3-(tert- butoxycarbonyl)bicyclo [3.2.1]octane-8- carboxylic acid |
| REF 373 | 2-[2,3-difluoro-4-[8- [4-[4-(4- hydroxypiperidine-4- carbonyl)piperazine- 1-carbonyl]-3-methyl- anilino]imidazo[1,2- a]pyrazin-3- yl]phenoxy]acetonitrile formate | 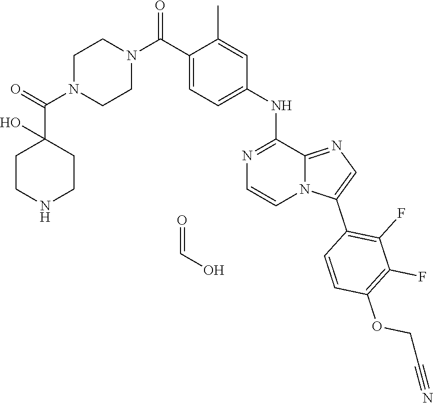 [SMILES: Cc1cc(Nc2nccn3c(-c4ccc(OCC#N)c(F)c4F)cnc23)ccc1C(=O)N1CCN(C(=O)C2(O)CCNCC2)CC1 ; O=CO] | 631.3 | Intermediate 109 and 1- (tert-butoxycarbonyl)- 4-hydroxypiperidine-4- carboxylic acid |
| REF 374 | N-[(2R)-2-[[(2S)-2- amino-5-guanidino- pentanoyl]amino]propyl]- 4-[[3-(2,3- difluoro-4-methoxy- phenyl)imidazo[1,2- a]pyrazin-8- yl]amino]-2-ethyl- benzamide formate | 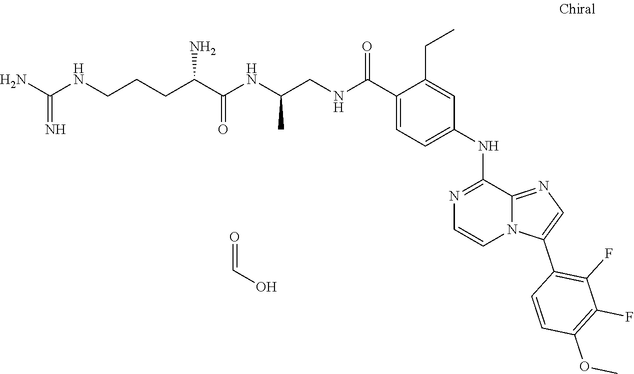 [SMILES: CCc1cc(Nc2nccn3c(-c4ccc(OC)c(F)c4F)cnc23)ccc1C(=O)NC[C@@H](C)NC(=O)[C@@H](N)CCCNC(=N)N ; O=CO] | 637.1 | Intermediate 104 and FMOC-ARG-OH. Deprotection using 22 eq piperidine at 0-20° C. for 12h. Purification by prep HPLC (formic acid as additive). |
| REF 375 | N-[(2S)-2-[[(2S)-2- amino-5-guanidino- pentanoyl]amino]propyl]- 4-[[3-(2,3- difluoro-4-methoxy- phenyl)imidazo[1,2- a]pyrazin-8- yl]amino]-2-ethyl- benzamide formate | 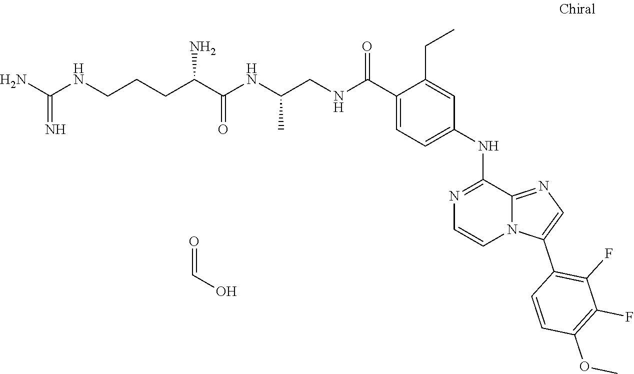 [SMILES: CCc1cc(Nc2nccn3c(-c4ccc(OC)c(F)c4F)cnc23)ccc1C(=O)NC[C@H](C)NC(=O)[C@@H](N)CCCNC(=N)N ; O=CO] | 637.1 | Intermediate 105 and FMOC-ARG-OH. Deprotection using 22 eq piperidine at 0-20° C. for 12h. Purification by prep HPLC (formic acid as additive). |
| REF 376 | N-[2-[[(2S)-2-amino- 5-guanidino- pentanoyl]amino]ethyl]- 4-[[3-[4- (cyanomethoxy)-2,3- difluoro- phenyl]imidazo[1,2- a]pyrazin-8- yl]amino]-2-ethyl- benzamide formate | 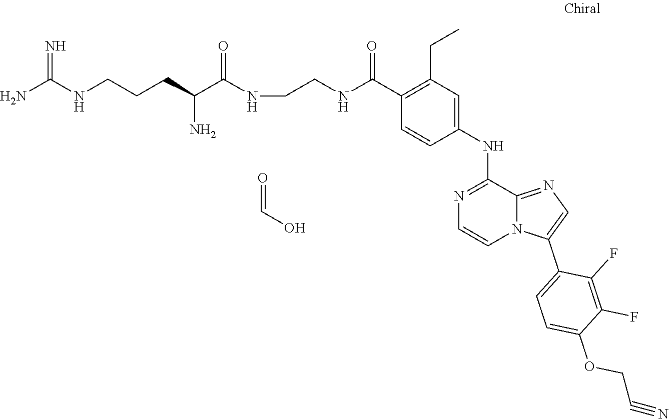 [SMILES: CCc1cc(Nc2nccn3c(-c4ccc(OCC#N)c(F)c4F)cnc23)ccc1C(=O)NCCNC(=O)[C@@H](N)CCCNC(=N)N ; O=CO] | 648.2 | Intermediate 116 and BOC-ARG-OH. Deprotection: 2h at rt in a 100/20 DCM/TFA mixture (200 eq TFA), then neutralized with sat. Na2CO3 to pH8. Purification: prep HPLC |
| REF 377 | 2-[3-chloro-2-fluoro- 4-[8-[4-[4-[(2R)-2- (hydroxymethyl) piperazine-1- carbonyl]piperidine-1- carbonyl]-3-methyl- anilino]imidazo[1,2- a]pyrazin-3- yl]phenoxy]acetonitrile | 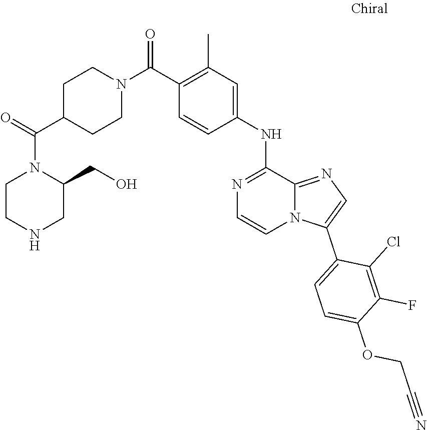 [SMILES: Cc1cc(Nc2nccn3c(-c4ccc(OCC#N)c(F)c4Cl)cnc23)ccc1C(=O)N1CCC(C(=O)N2CCNC[C@@H]2CO)CC1] | 661.3 | Intermediate 111 and 124. Deprotection: 1h at rt in a 100/10 DCM/TFA mixture (160 eq TFA), Purification: prep HPLC |
| REF 378 | 2-[3-chloro-2-fluoro- 4-[8-[4-[4-[(2S)-2- (hydroxymethyl) piperazine-1- carbonyl]piperidine-1- carbonyl]-3-methyl- anilino]imidazo[1,2- a]pyrazin-3- yl]phenoxy]acetonitrile formate | 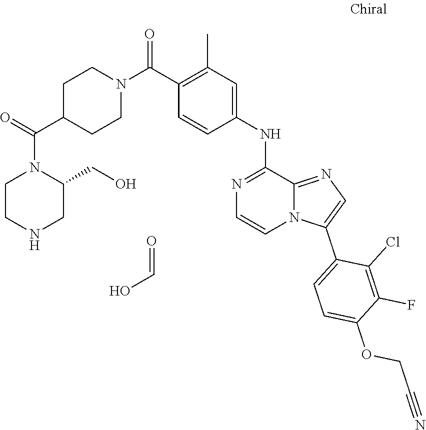 [SMILES: Cc1cc(Nc2nccn3c(-c4ccc(OCC#N)c(F)c4Cl)cnc23)ccc1C(=O)N1CCC(C(=O)N2CCNC[C@H]2CO)CC1 ; O=CO] | 661.2 | Intermediate 111 and Intermediate 125. Deprotection: 1 h at rt in a 100/10 DCM/TFA mixture (160 eq TFA), Purification: prep HPLC |
| REF 379 | N-[2-[[(2R)-2-amino- 5-guanidino- pentanoyl]amino]ethyl | 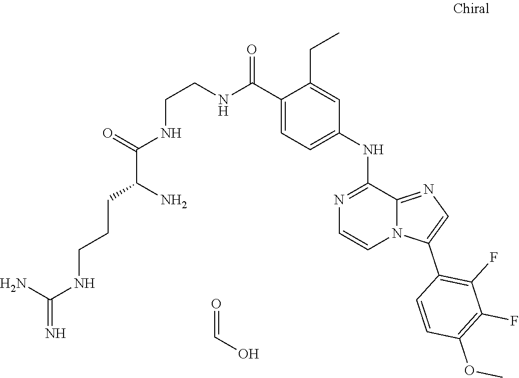 [SMILES: CCc1cc(Nc2nccn3c(-c4ccc(OC)c(F)c4F)cnc23)ccc1C(=O)NCCNC(=O)[C@H](N)CCCNC(=N)N ; O=CO] | 623.1 | Reference Example 296 and FMOC-D- ARG-OH. Deprotection using 22 eq piperidine at 0-20° C. for 12h. Purification by prep HPLC. |
| REF 380 | N-[2-[[(2S)-2-amino- 5-guanidino- pentanoyl]amino]ethyl]- 4-[[3-(2-chloro-3- fluoro-4-methoxy- phenyl)imidazo[1,2- a]pyrazin-8- yl]amino]-2-ethyl- benzamide formate | 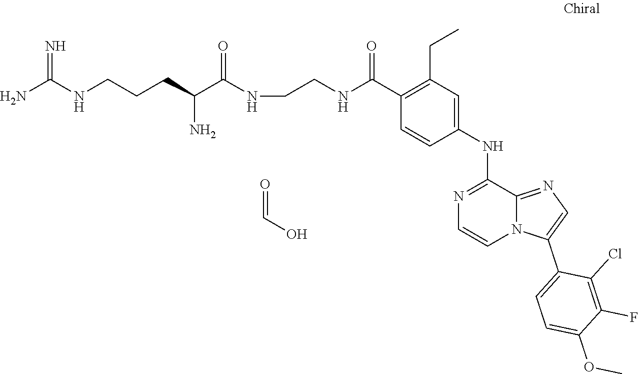 [SMILES: CCc1cc(Nc2nccn3c(-c4ccc(OC)c(F)c4Cl)cnc23)ccc1C(=O)NCCNC(=O)[C@@H](N)CCCNC(=N)N ; O=CO] | 639.3 | Intermediate 117 and BOC-ARG-OH. Deprotection: 2h at rt in a 100/10 DCM/TFA mixture (38 eq TFA). Purification: prep HPLC |
| REF 381 | 2-[3-chloro-2-fluoro- 4-[8-[3-methyl-4-[4- (piperidine-4- carbonyl)piperazine- 1- carbonyl]anilino]imidazo [1,2-a]pyrazin-3- yl]phenoxy]acetonitrile formate | 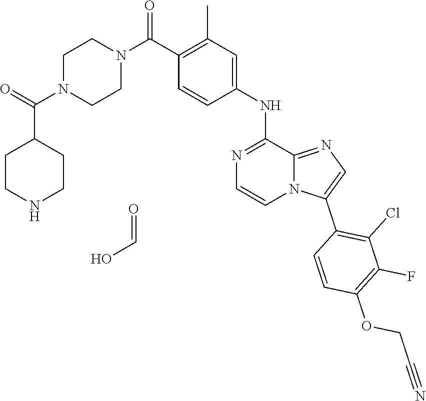 [SMILES: Cc1cc(Nc2nccn3c(-c4ccc(OCC#N)c(F)c4Cl)cnc23)ccc1C(=O)N1CCN(C(=O)C2CCNCC2)CC1 ; O=CO] | 631.3 | Intermediate 111 and N-BOC-isonipecotic acid. Deprotection: 1h at rt in a 100/10 DCM/TFA mixture. Purification: prep HPLC. |
| REF 382 | N-[2-[[(2R)-2-amino- 5-guanidino- pentanoyl]amino]ethyl]- 4-[[3-[2-chloro-4- (cyanomethoxy)-3- fluoro- phenyl]imidazo[1,2- a]pyrazin-8- yl]amino]-2-ethyl- benzamide formate | 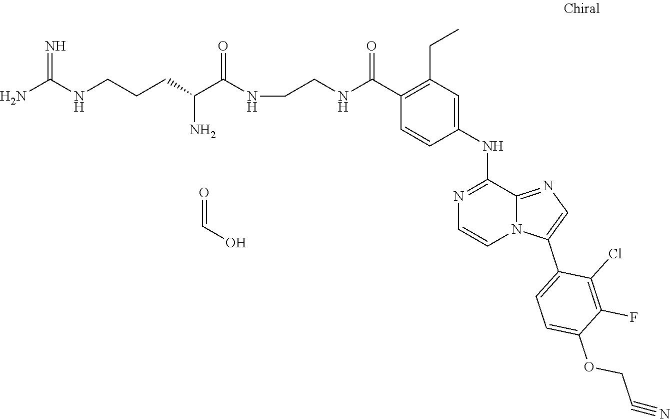 [SMILES: CCc1cc(Nc2nccn3c(-c4ccc(OCC#N)c(F)c4Cl)cnc23)ccc1C(=O)NCCNC(=O)[C@H](N)CCCNC(=N)N ; O=CO] | 664.1 | Intermediate 118 and FMOC-D-ARG-OH. Deprotection using 22 eq piperidine at 0-20° C. for 12h. Purification by prep HPLC. |
| REF 383 | [4-[[3-(2-chloro-3- fluoro-4-methoxy- phenyl)imidazo[1,2- a]pyrazin-8- yl]amino]-2-methyl- phenyl]-[4-[(3S)- pyrrolidine-3- carbonyl]piperazin-1- yl]methanone formate | 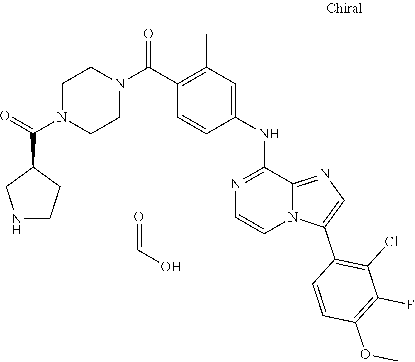 [SMILES: COc1ccc(-c2cnc3c(Nc4ccc(C(=O)N5CCN(C(=O)[C@H]6CCNC6)CC5)c(C)c4)nccn23)c(Cl)c1F ; O=CO] | 592.1 | Intermediate 103 and (S)-1-BOC- pyrrolidine-3- carboxylic acid. Deprotection: 1h at rt in a 100/10 DCM/TFA mixture. Purification: prep HPLC |
| REF 384 | 2-[3-chloro-2-fluoro- 4-[8-[4-[4-[(2S,4R)-4- hydroxypyrrolidine-2- carbonyl]piperazine- 1-carbonyl]-3-methyl- anilino]imidazo[1,2- a]pyrazin-3- yl]phenoxy]acetonitrile formate | 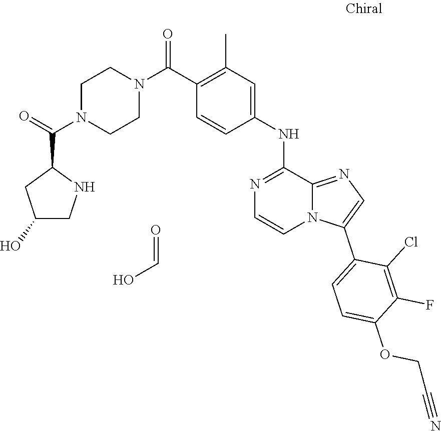 [SMILES: Cc1cc(Nc2nccn3c(-c4ccc(OCC#N)c(F)c4Cl)cnc23)ccc1C(=O)N1CCN(C(=O)[C@@H]2C[C@@H](O)CN2)CC1 ; O=CO] | 633.3 | Intermediate 111 and (2R,4S)-1-tert- butoxycarbonyl-4- hydroxy-pyrrolidine- 2-carboxylic acid. Deprotection: 1h at rt in a 10/1 DCM/TFA mixture (95 eq TFA). Purification: prep. HPLC |
| REF 385 | 2-[3-chloro-2-fluoro- 4-[8-[3-methyl-4-[4- [(3R)-pyrrolidine-3- carbonyl]piperazine- 1- carbonyl]anilino]imidazo [1,2-a]pyrazin-3- yl]phenoxy]acetonitrile formate | 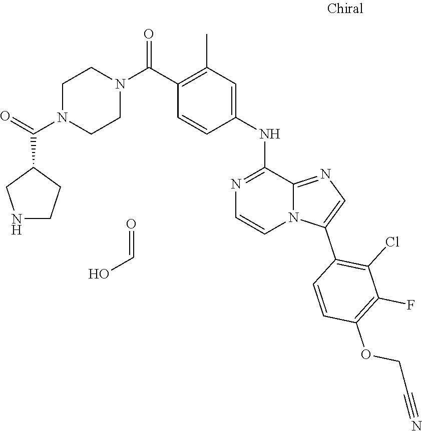 [SMILES: Cc1cc(Nc2nccn3c(-c4ccc(OCC#N)c(F)c4Cl)cnc23)ccc1C(=O)N1CCN(C(=O)[C@@H]2CCNC2)CC1 ; O=CO] | 617.3 | Intermediate 111 and (R)-1-BOC- pyrrolidine-3- carboxylic acid. Deprotection: 1h at rt in a 10/1 DCM/TFA mixture Purification: prep. HPLC |
| REF 386 | 2-[3-chloro-2-fluoro- 4-[8-[3-methyl-4-[4- [(3S)-pyrrolidine-3- carbonyl]piperazine- 1- carbonyl]anilino]imidazo [1,2-a]pyrazin-3- yl]phenoxy]acetonitrile formate | 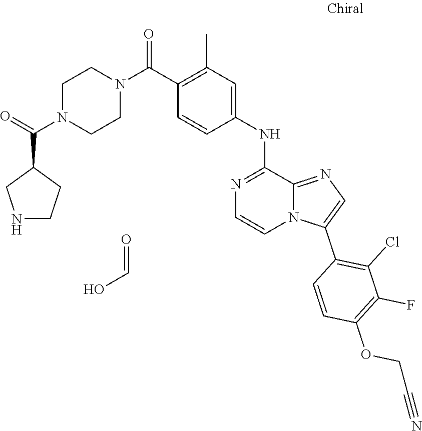 [SMILES: Cc1cc(Nc2nccn3c(-c4ccc(OCC#N)c(F)c4Cl)cnc23)ccc1C(=O)N1CCN(C(=O)[C@H]2CCNC2)CC1 ; O=CO] | 617.3 | Intermediate 111 and (S)-1-BOC-pyrrolidine- 3-carboxylic acid. Deprotection: 1h at rt in a 10/1 DCM/TFA mixture Purification: prep. HPLC |
| REF 387 | N-[2-[[(2S)-2-amino- 5-(4,5-dihydro-1H- imidazol-2- ylamino)pentanoyl] amino]ethyl]-4-[[3-(2,3- difluoro-4-methoxy- phenyl)imidazo[1,2- a]pyrazin-8- yl]amino]-2-ethyl- benzamide formate | 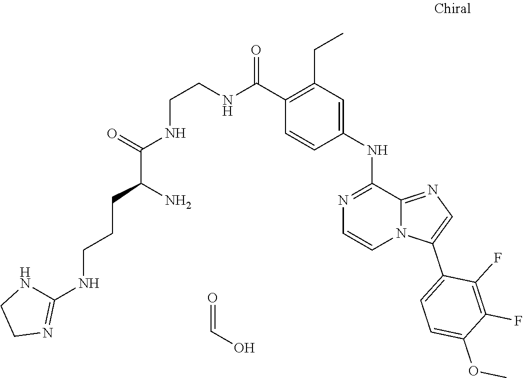 [SMILES: CCc1cc(Nc2nccn3c(-c4ccc(OC)c(F)c4F)cnc23)ccc1C(=O)NCCNC(=O)[C@@H](N)CCCNC1=NCCN1 ; O=CO] | 649.4 | Reference Example 296 and (2S)-2-(tert- butoxycarbonylamino)- 5-(4,5-dihydro-1H- imidazol-2- ylamino)pentanoic acid. Purification: prep. HPLC |
| REF 388 | N-[2-[[(2R)-2-amino- 5-guanidino- pentanoyl]amino]ethyl]- 4-[[3-(2-chloro-3- fluoro-4-methoxy- phenyl)imidazo[1,2- a]pyrazin-8- yl]amino]-2-ethyl- benzamide formate | 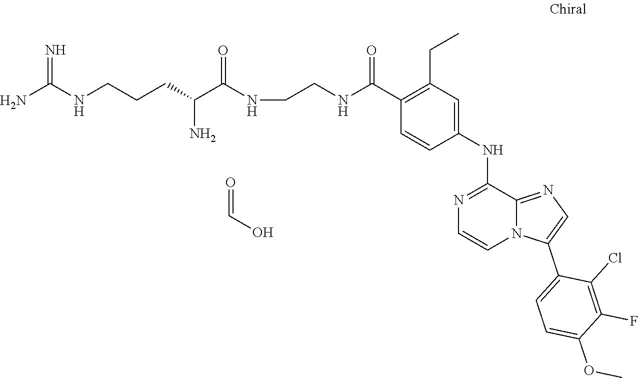 [SMILES: CCc1cc(Nc2nccn3c(-c4ccc(OC)c(F)c4Cl)cnc23)ccc1C(=O)NCCNC(=O)[C@H](N)CCCNC(=N)N ; O=CO] | 639.4 | Intermediate 117 and FMOC-D-ARG-OH. Deprotection using 22 eq piperidine at 20° . for 12h. Purification by prep HPLC. |
| REF 389 | N-[2-[[(2R)-2-amino- 5-guanidino- pentanoyl]amino]ethyl]- 4-[[3-(4-chloro-2,3- difluoro- phenyl)imidazo[1,2- a]pyrazin-8- yl]amino]-2-ethyl- benzamide formate | 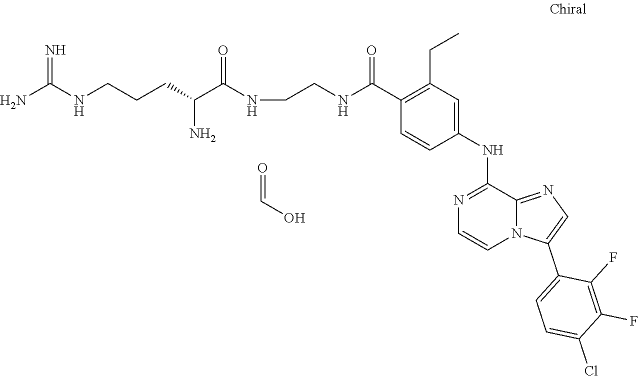 [SMILES: CCc1cc(Nc2nccn3c(-c4ccc(Cl)c(F)c4F)cnc23)ccc1C(=O)NCCNC(=O)[C@H](N)CCCNC(=N)N ; O=CO] | 627.3 | Intermediate 119 and FMOC-D-ARG-OH. Deprotection using 22 eq piperidine at 20° C. for 12h. Purification by prep HPLC. |
| REF 390 | 2-[3-chloro-2-fluoro- 4-[8-[4-[4-(4- hydroxypiperidine-4- carbonyl)piperazine- 1-carbonyl]-3-methyl- anilino]imidazo[1,2- a]pyrazin-3- yl]phenoxy]acetonitrile formate | 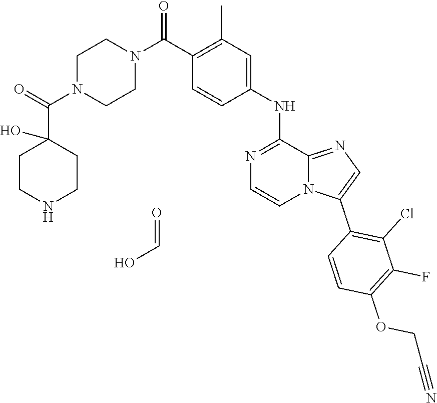 [SMILES: Cc1cc(Nc2nccn3c(-c4ccc(OCC#N)c(F)c4Cl)cnc23)ccc1C(=O)N1CCN(C(=O)C2(O)CCNCC2)CC1 ; O=CO] | 647.3 | Intermediate 111 and 1- (tert-butoxycarbonyl)- 4-hydroxypiperidine-4- carboxylic acid. Deprotection: 1h at rt in a 10/1 DCM/TFA mixture Purification: prep. HPLC. |
| REF 391 | N-[2-[[(2S)-2-amino- 4-guanidino- butanoyl]amino]ethyl]- 4-[[3-(2,3-difluoro-4- methoxy- phenyl)imidazo[1,2- a]pyrazin-8- yl]amino]-2-ethyl- benzamide formate | 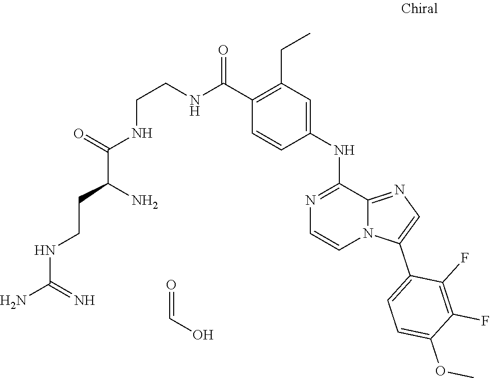 [SMILES: CCc1cc(Nc2nccn3c(-c4ccc(OC)c(F)c4F)cnc23)ccc1C(=O)NCCNC(=O)[C@@H](N)CCNC(=N)N ; O=CO] | 609.2 | Reference Example 296 and (2S)-2-(tert- butoxycarbonylamino)- 4-guanidino-butanoic acid. Purification: prep. HPLC |
| REF 392 | [4-[[3-(2-chloro-3- fluoro-4-methoxy- phenyl)imidazo[1,2- a]pyrazin-8- yl]amino]-2-methyl- phenyl]-[4-(4- hydroxypiperidine-4- carbonyl)piperazin-1- yl]methanone formate | 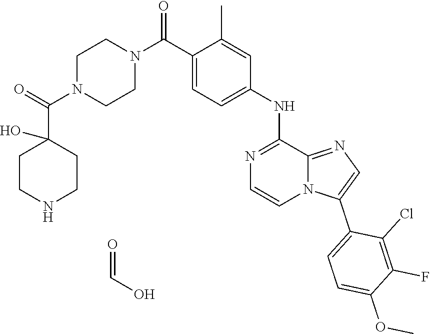 [SMILES: COc1ccc(-c2cnc3c(Nc4ccc(C(=O)N5CCN(C(=O)C6(O)CCNCC6)CC5)c(C)c4)nccn23)c(Cl)c1F ; O=CO] | 622.1 | Intermediate 103 and 1-(tert- butoxycarbonyl)-4- hydroxypiperidine-4- carboxylic acid. Purification: prep. HPLC |
| REF 393 | [4-[[3-(2-chloro-3- fluoro-4-methoxy- phenyl)imidazo[1,2- a]pyrazin-8- yl]amino]-2-methyl- phenyl]-[4-[(3S,4R)- 3-hydroxypiperidine- 4-carbonyl]piperazin- 1-yl]methanone formate | 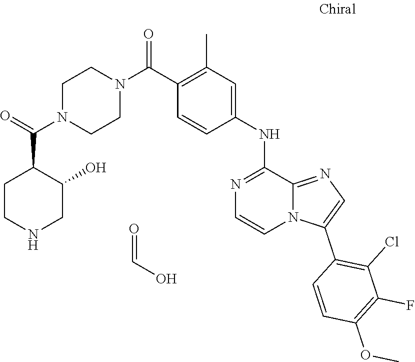 [SMILES: COc1ccc(-c2cnc3c(Nc4ccc(C(=O)N5CCN(C(=O)[C@@H]6CCNC[C@H]6O)CC5)c(C)c4)nccn23)c(Cl)c1F ; O=CO] | 622.2 | Intermediate 103 and (3S,4R)-1-tert- butoxycarbonyl-3- hydroxy-piperidine-4- carboxylic acid obtained by saponification of 1,4- Piperidinedicarboxylic acid, 3-hydroxy-, 1- (1,1-dimethylethyl) 4- ethyl ester, (3S,4R)- [CAS# 1805790-50-4]. |
| REF 394 | [4-[[3-(2-chloro-3- fluoro-4-methoxy- phenyl)imidazo[1,2- a]pyrazin-8- yl]amino]-2-methyl- phenyl]-[4-[(3S,4S)-3- hydroxypiperidine-4- carbonyl]piperazin-1- yl]methanone | 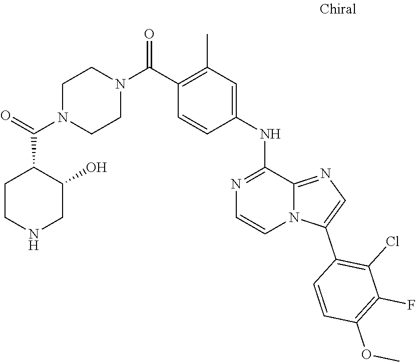 [SMILES: COc1ccc(-c2cnc3c(Nc4ccc(C(=O)N5CCN(C(=O)[C@H]6CCNC[C@H]6O)CC5)c(C)c4)nccn23)c(Cl)c1F] | 622.2 | Intermediate 103 and (3S,4S)-1-tert- butoxycarbonyl-3- hydroxy-piperidine-4- carboxylic acid, obtained by saponification of 1,4- piperidinedicarboxylic acid, 3-hydroxy-, 1- (1,1-dimethylethyl) 4- methyl ester, (3S,4S)- [CAS# 2166250-53-7] |
| REF 395 | [4-[[3-(2-chloro-3- fluoro-4-methoxy- phenyl)imidazo[1,2- a]pyrazin-8- yl]amino]-2-methyl- phenyl]-[4-[(3R,4R)- 3-hydroxypiperidine- 4-carbonyl]piperazin- 1-yl]methanone | 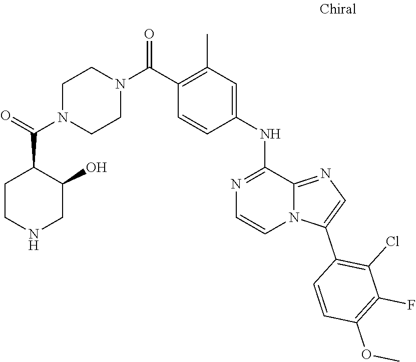 [SMILES: COc1ccc(-c2cnc3c(Nc4ccc(C(=O)N5CCN(C(=O)[C@@H]6CCNC[C@@H]6O)CC5)c(C)c4)nccn23)c(Cl)c1F] | 622.2 | Intermediate 103 and 1,4- piperidinedicarboxylic acid, 3-hydroxy-, 1-(1, 1-dimethylethyl) ester, (3R,4R)-rel- [CAS# 206111-42-4]. |
| REF 396 | [4-[[3-(2-chloro-3- fluoro-4-methoxy- phenyl)imidazo[1,2- a]pyrazin-8- yl]amino]-2-methyl- phenyl]-[4-[(3R,4S)- 3-hydroxypiperidine- 4-carbonyl]piperazin- 1-yl]methanone formate | 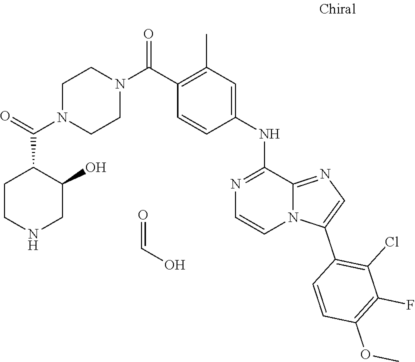 [SMILES: COc1ccc(-c2cnc3c(Nc4ccc(C(=O)N5CCN(C(=O)[C@H]6CCNC[C@@H]6O)CC5)c(C)c4)nccn23)c(Cl)c1F ; O=CO] | 622.2 | Intermediate 103 and 1,4- piperidinedicarboxylic acid, 3-hydroxy-, 1- (1,1-dimethylethyl) ester, (3R,4S)- [CAS# 1821775-81-8]. Purification: prep. HPLC |
| REF 397 | N-[2-[[(2R)-2-amino- 5-guanidino- pentanoyl]amino]ethyl]- 4-[[3-[4- (difluoromethoxy)- 2,3-difluoro- phenyl]imidazo[1,2- a]pyrazin-8- yl]amino]-2-ethyl- benzamide formate | 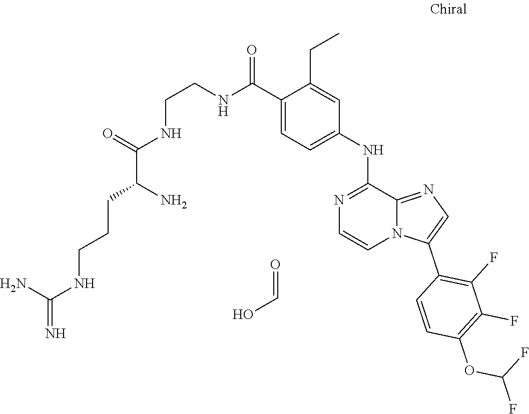 [SMILES: CCc1cc(Nc2nccn3c(-c4ccc(OC(F)F)c(F)c4F)cnc23)ccc1C(=O)NCCNC(=O)[C@H](N)CCCNC(=N)N ; O=CO] | 659.2 | Intermediate 120 and (2R)-2-(tert- butoxycarbonylamino)- 5-guanidino-pentanoic acid hydrate hydrochloride |
| REF 398 | 2-[3-chloro-2-fluoro- 4-[8-[4-[4-[(3R,4S)-3- hydroxypiperidine-4- carbonyl]piperazine- 1-carbonyl]-3-methyl- anilino]imidazo[1,2- a]pyrazin-3- yl]phenoxy]acetonitrile formate | 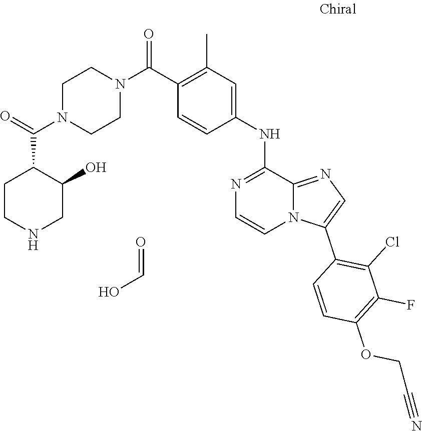 [SMILES: Cc1cc(Nc2nccn3c(-c4ccc(OCC#N)c(F)c4Cl)cnc23)ccc1C(=O)N1CCN(C(=O)[C@H]2CCNC[C@@H]2O)CC1 ; O=CO] | 647.2 | Intermediate 111 and 1,4- Piperidinedicarboxylic acid, 3-hydroxy-, 1- (1,1-dimethylethyl) ester, (3R,4S)- [CAS# 1821775-81-8]. Deprotection: 1h at rt in a 10/1 DCM/TFA mixture Purification: prep. HPLC |
| REF 399 | 2-[3-chloro-2-fluoro- 4-[8-[4-[4-[(3S,4R)-3- hydroxypiperidine-4- carbonyl]piperazine- 1-carbonyl]-3-methyl- anilino]imidazo[1,2- a]pyrazin-3- yl]phenoxy]acetonitrile formate | 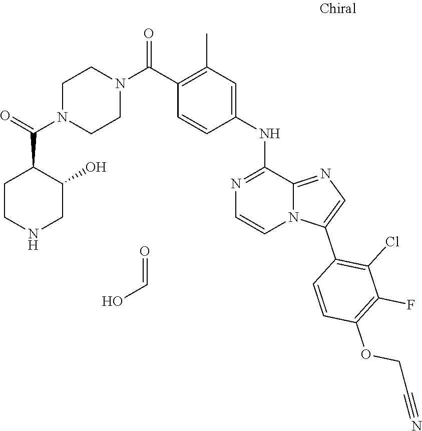 [SMILES: Cc1cc(Nc2nccn3c(-c4ccc(OCC#N)c(F)c4Cl)cnc23)ccc1C(=O)N1CCN(C(=O)[C@@H]2CCNC[C@H]2O)CC1 ; O=CO] | 647.2 | Intermediate 111 and (3S,4R)-1-tert- butoxycarbonyl-3- hydroxy-piperidine-4- carboxylic acid obtained by saponification of 1,4- Piperidinedicarboxylic acid, 3-hydroxy-, 1- (1,1-dimethylethyl) 4- ethyl ester, (3S,4R)- [CAS# 1805790-50-4]. Deprotection: 1h at rt in a 10/1 DCM/TFA mixture Purification: prep HPLC (FA as additive) |
| REF 400 | 2-[3-chloro-2-fluoro- 4-[8-[4-[4-[(3S,4S)-3- hydroxypiperidine-4- carbonyl]piperazine- 1-carbonyl]-3-methyl- anilino]imidazo[1,2- a]pyrazin-3- yl]phenoxy]acetonitrile formate | 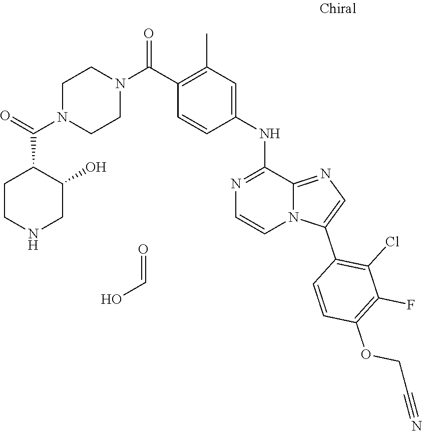 [SMILES: Cc1cc(Nc2nccn3c(-c4ccc(OCC#N)c(F)c4Cl)cnc23)ccc1C(=O)N1CCN(C(=O)[C@H]2CCNC[C@H]2O)CC1 ; O=CO] | 647.2 | Intermediate 111 and (3S,4S)-1-tert- butoxycarbonyl-3- hydroxy-piperidine-4- carboxylic acid, obtained by saponification of 1,4- Piperidinedicarboxylic acid, 3-hydroxy-, 1- (1,1-dimethylethyl) 4- methyl ester, (3S,4S)- [CAS# 2166250-53-7]. Deprotection: 1h at rt in a 10/1 DCM/TFA mixture Purification: prep HPLC (FA as additive) |
| REF 401 | 2-[3-chloro-2-fluoro- 4-[8-[4-[4-[(3R,4R)-3- hydroxypiperidine-4- carbonyl]piperazine- 1-carbonyl]-3-methyl- anilino]imidazo[1,2- a]pyrazin-3- yl]phenoxy]acetonitrile formate | 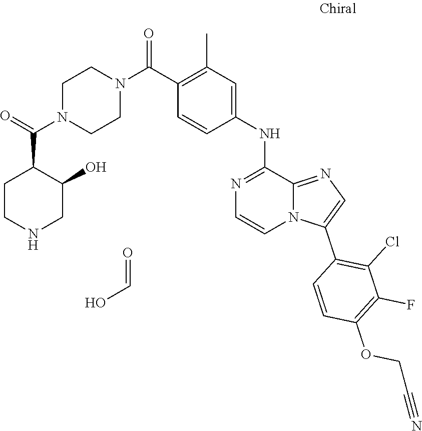 [SMILES: Cc1cc(Nc2nccn3c(-c4ccc(OCC#N)c(F)c4Cl)cnc23)ccc1C(=O)N1CCN(C(=O)[C@@H]2CCNC[C@@H]2O)CC1 ; O=CO] | 647.2 | Intermediate 111 and 1,4- Piperidinedicarboxylic acid, 3-hydroxy-, 1- (1,1-dimethylethyl) ester, (3R,4R)-rel- [CAS# 206111-42-4]. Deprotection: 1h at rt in a 10/1 DCM/TFA mixture Purification: prep HPLC (FA as additive) |
| REF 402 | 1-(4-(4-((3-(3-fluoro- 4- methoxyphenyl)imidazo [1,2-a]pyrazin-8- yl)amino)-2- methylbenzoyl)piperazin- 1-yl)-2- (methylamino)ethanone hydrochloride | 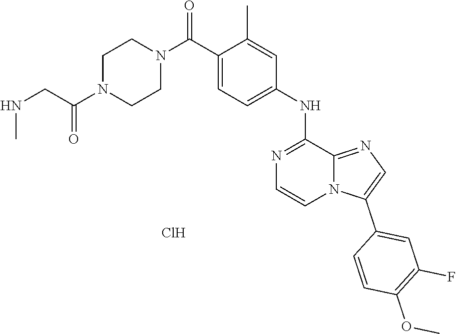 [SMILES: CNCC(=O)N1CCN(C(=O)c2ccc(Nc3nccn4c(-c5ccc(OC)c(F)c5)cnc34)cc2C)CC1 ; Cl] | 532.2 | Reference Example 295 and 2-((tert- butoxycarbonyl)(methyl) amino)acetic acid |
| REF 403 | 1-(3-(4-(4-((3-(4- (difluoromethoxy) phenyl)imidazo[1,2- a]pyrazin-8- yl)amino)-2- methylbenzoyl) piperazin-1-yl)-3- oxopropyl)guanidine | 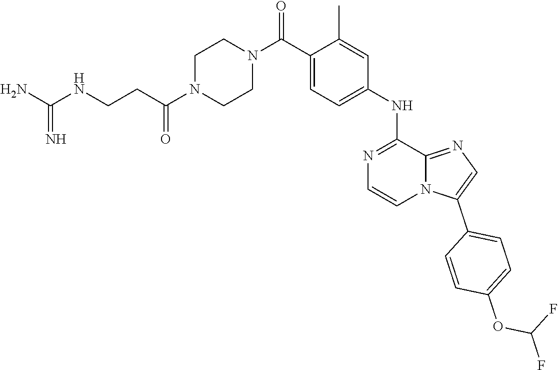 [SMILES: Cc1cc(Nc2nccn3c(-c4ccc(OC(F)F)cc4)cnc23)ccc1C(=O)N1CCN(C(=O)CCNC(=N)N)CC1] | 592.3 | Reference Example 294 and 3- guanidinopropanoic acid |
| REF 404 | (2S)-2-amino-1-[4-[4- [[3-[4- (difluoromethoxy) phenyl]imidazo[1,2- a]pyrazin-8- yl]amino]-2-methyl- benzoyl]piperazin-1- yl]propan-1-one hydrochloride | 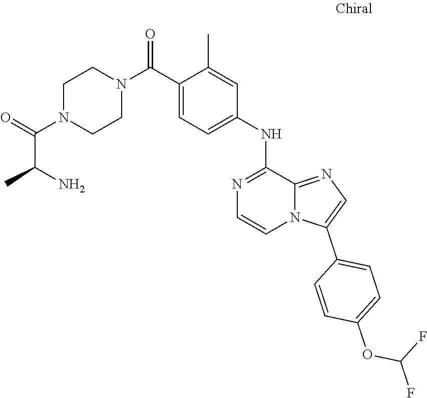 [SMILES: Cc1cc(Nc2nccn3c(-c4ccc(OC(F)F)cc4)cnc23)ccc1C(=O)N1CCN(C(=O)[C@H](C)N)CC1] | [M − H]−; 584.4 | Reference Example 294 and N-Boc-L- Alanine |
| REF 405 | (2S,4S)-N-[2-[[4-[[3- [4-(cyanomethoxy)- 2,3-difluoro- phenyl]imidazo[1,2- a]pyrazin-8- yl]amino]-2-methyl- benzoyl]amino]ethyl]- 4-hydroxy-4-methyl- pyrrolidine-2- carboxamide formate | 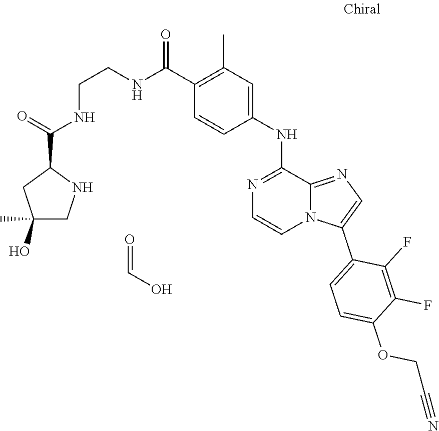 [SMILES: Cc1cc(Nc2nccn3c(-c4ccc(OCC#N)c(F)c4F)cnc23)ccc1C(=O)NCCNC(=O)[C@@H]1C[C@](C)(O)CN1 ; O=CO] | 605.2 | Intermediate 121 and 1,2- Pyrrolidinedicarboxylic acid, 4-hydroxy-4- methyl-, 1-(1,1- dimethylethyl) ester, (2S,4S)- [CAS#1199793-52-6] |
| REF 406 | (2S,4S)-N-[3-[[4-[[3- [4-(cyanomethoxy)- 2,3-difluoro- phenyl]imidazo[1,2- a]pyrazin-8- yl]amino]-2-methyl- benzoyl]amino]propyl]- 4-hydroxy-4-methyl- pyrrolidine-2- carboxamide formate | 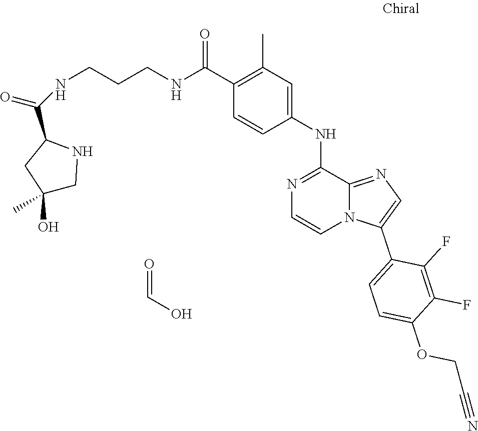 [SMILES: Cc1cc(Nc2nccn3c(-c4ccc(OCC#N)c(F)c4F)cnc23)ccc1C(=O)NCCCNC(=O)[C@@H]1C[C@](C)(O)CN1 ; O=CO] | 619.1 | Intermediate 122 and 1,2- Pyrrolidinedicarboxylic acid, 4-hydroxy-4- methyl-, 1-(1,1- dimethylethyl) ester, (2S,4S)- [CAS#1199793-52-6] |
| REF 407 | [4-[[3-(2,3-difluoro-4- methoxy- phenyl)imidazo[1,2- a]pyrazin-8- yl]amino]-2-methyl- phenyl]-[4-[(2S,4S)-4- hydroxypyrrolidine-2- carbonyl]piperazin-1- yl]methanone | 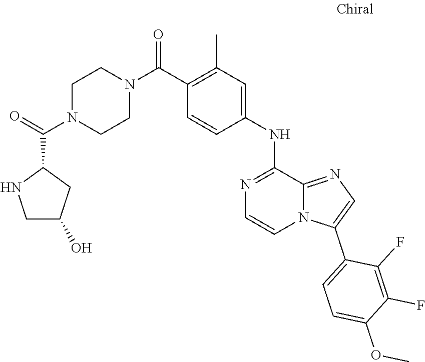 [SMILES: COc1ccc(-c2cnc3c(Nc4ccc(C(=O)N5CCN(C(=O)[C@@H]6C[C@H](O)CN6)CC5)c(C)c4)nccn23)c(F)c1F] | [M − H]−; 590.0 | Reference Example 208 and (2S,4S)-1- (tert-butoxycarbonyl)- 4-hydroxypyrrolidine- 2-carboxylic acid [CAS#87691-27-8] |
| REF 408 | [4-[[3-(2,3-difluoro-4- methoxy- phenyl)imidazo[1,2- a]pyrazin-8- yl]amino]-2-methyl- phenyl]-[4-[rac- (2R,4S)-4- hydroxypyrrolidine-2- carbonyl]piperazin-1- yl]methanone | 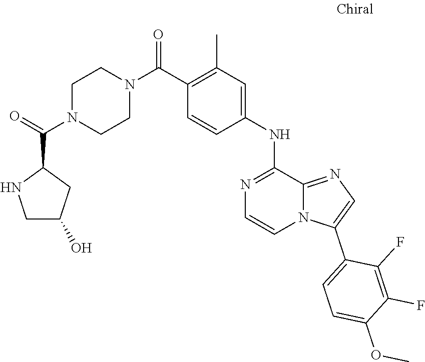 [SMILES: COc1ccc(-c2cnc3c(Nc4ccc(C(=O)N5CCN(C(=O)[C@H]6C[C@H](O)CN6)CC5)c(C)c4)nccn23)c(F)c1F] | [M − H]−; 590.4 | Reference Example 208 and (2R,4S)-1- (tert-butoxycarbonyl)- 4-hydroxypyrrolidine- 2-carboxylic acid [CAS#147266-92-0] |
| REF 409 | 2-[4-[8-[4-[4- [(2S,4S)-4-ethyl-4- hydroxy-pyrrolidine- 2- carbonyl]piperazine- 1-carbonyl]-3-methyl- anilino]imidazo[1,2- a]pyrazin-3-yl]-2,3- difluoro- phenoxy]acetonitrile formate | 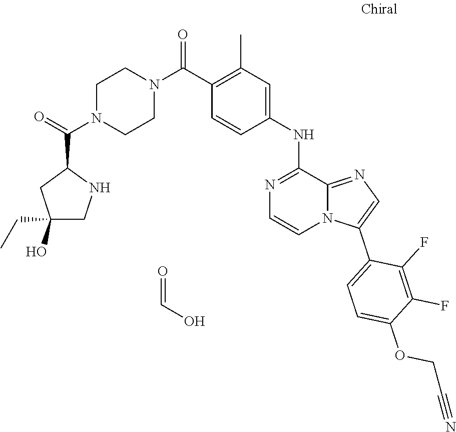 [SMILES: CC[C@@]1(O)CN[C@H](C(=O)N2CCN(C(=O)c3ccc(Nc4nccn5c(-c6ccc(OCC#N)c(F)c6F)cnc45)cc3C)CC2)C1 ; O=CO] | 645.5 | Intermediate 109 and Intermediate 126 |
| REF 410 | 2-[2,3-difluoro-4-[8- [4-[4-[(2S,4S)-4- hydroxy-4-methyl- pyrrolidine-2- carbonyl]piperazine- 1-carbonyl]-3-methyl- anilino]imidazo[1,2- a]pyrazin-3- yl]phenoxy]acetonitrile formate | 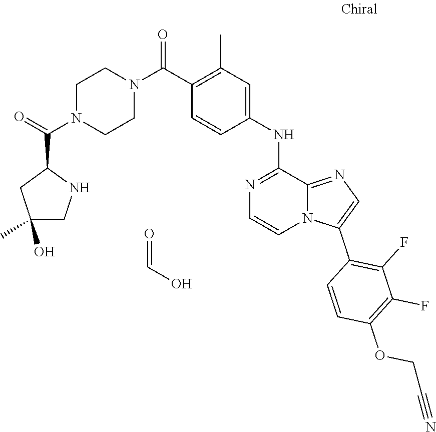 [SMILES: Cc1cc(Nc2nccn3c(-c4ccc(OCC#N)c(F)c4F)cnc23)ccc1C(=O)N1CCN(C(=O)[C@@H]2C[C@](C)(O)CN2)CC1 ; O=CO] | 631.2 | Intermediate 109 and 1, 2-pyrrolidinedicarboxylic acid, 4-hydroxy-4- methyl-, 1-(1,1- dimethylethyl) ester, (2S,4S)- [CAS#1199793-52-6] |
| REF 411 | 2-[2,3-difluoro-4-[8- [4-[4-[(2S,4R)-4- hydroxy-4-methyl- pyrrolidine-2- carbonyl]piperazine- 1-carbonyl]-3-methyl- anilino]imidazo[1,2- a]pyrazin-3- yl]phenoxy]acetonitrile formate | 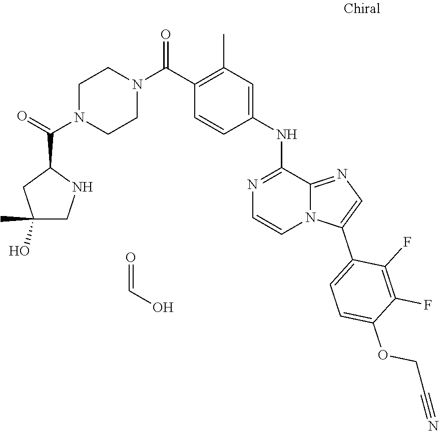 [SMILES: Cc1cc(Nc2nccn3c(-c4ccc(OCC#N)c(F)c4F)cnc23)ccc1C(=O)N1CCN(C(=O)[C@@H]2C[C@@](C)(O)CN2)CC1 ; O=CO] | 631.4 | Intermediate 109 and 1, 2-Pyrrolidinedicarboxylic acid, 4-hydroxy-4- methyl-, 1-(1,1- dimethylethyl) ester, (2S,4R)- [CAS#1365970-67-7] |
| REF 412 | 2-[4-[8-[4-[4- [(2S,4R)-4-ethyl-4- hydroxy-pyrrolidine- 2- carbonyl]piperazine- 1-carbonyl]-3-methyl- anilino]imidazo[1,2- a]pyrazin-3-yl]-2,3- difluoro- phenoxy]acetonitrile formate | 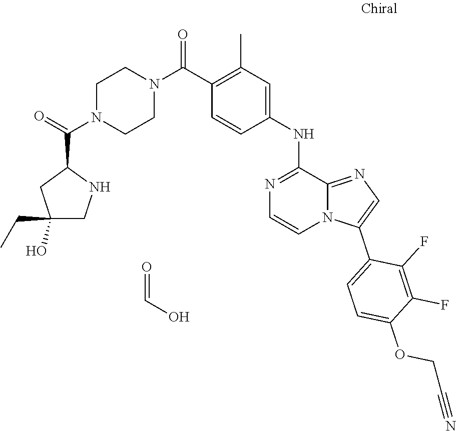 [SMILES: CC[C@]1(O)CN[C@H](C(=O)N2CCN(C(=O)c3ccc(Nc4nccn5c(-c6ccc(OCC#N)c(F)c6F)cnc45)cc3C)CC2)C1 ; O=CO] | 645.4 | Intermediate 109 and Intermediate 127 |
| REF 413 | N-[2-[[(2S)-2-amino- 3-hydroxy- propanoyl]amino]ethyl]- 4-[[3-[4- (cyanomethoxy)-2,3- difluoro- phenyl]imidazo[1,2- a]pyrazin-8- yl]amino]-2-ethyl- benzamide formate | 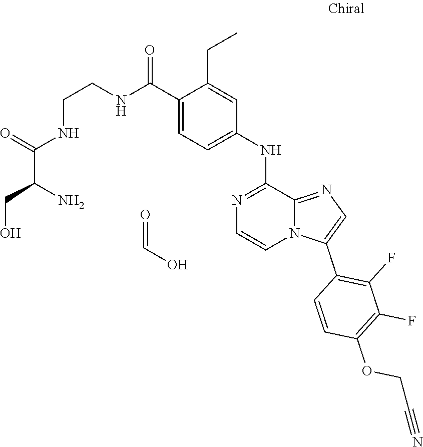 [SMILES: CCc1cc(Nc2nccn3c(-c4ccc(OCC#N)c(F)c4F)cnc23)ccc1C(=O)NCCNC(=O)[C@@H](N)CO ; O=CO] | 579.4 | Intermediate 116 and BOC-SER-OH |
| REF 414 | N-[2-[(2- aminoacetyl)amino] ethyl]-4-[[3-[4- (cyanomethoxy)-2,3- difluoro- phenyl]imidazo[1,2- a]pyrazin-8- yl]amino]-2-ethyl- benzamide formate | 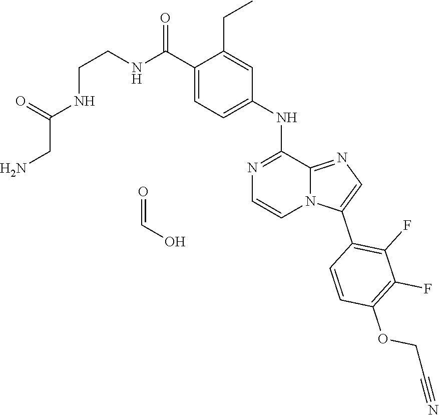 [SMILES: CCc1cc(Nc2nccn3c(-c4ccc(OCC#N)c(F)c4F)cnc23)ccc1C(=O)NCCNC(=O)CN ; O=CO] | 549.4 | Intermediate 116 and BOC-glycine |
| REF 415 | N-[2-(3- aminopropanoylamino) ethyl]-4-[[3-[4- (cyanomethoxy)-2,3- difluoro- phenyl]imidazo[1,2- a]pyrazin-8- yl]amino]-2-ethyl- benzamide formate | 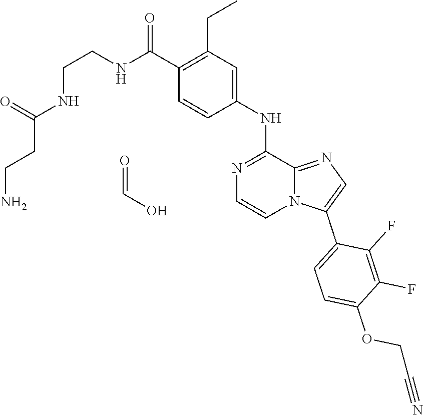 [SMILES: CCc1cc(Nc2nccn3c(-c4ccc(OCC#N)c(F)c4F)cnc23)ccc1C(=O)NCCNC(=O)CCN ; O=CO] | 563.3 | Intermediate 116 and BOC-BETA-ALA-OH |
| REF 416 | N-[2-[[(2S,3R)-2- amino-3-hydroxy- butanoyl]amino]ethyl]- 4-[[3-[4- (cyanomethoxy)-2,3- difluoro- phenyl]imidazo[1,2- a]pyrazin-8- yl]amino]-2-ethyl- benzamide formate | 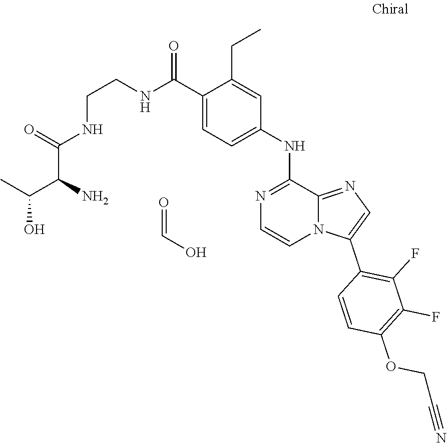 [SMILES: CCc1cc(Nc2nccn3c(-c4ccc(OCC#N)c(F)c4F)cnc23)ccc1C(=O)NCCNC(=O)[C@@H](N)[C@@H](C)O ; O=CO] | 593.3 | Intermediate 116 and BOC-THR-OH |
| REF 417 | 4-[[3-(2,3-difluoro-4- methoxy- phenyl)imidazo[1,2- a]pyrazin-8- yl]amino]-2-ethyl-N- [2-(5- guanidinopentanoyl- amino)ethyl]benzamide formate | 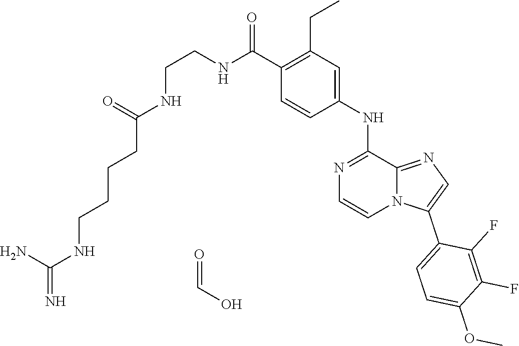 [SMILES: CCc1cc(Nc2nccn3c(-c4ccc(OC)c(F)c4F)cnc23)ccc1C(=O)NCCNC(=O)CCCCNC(=N)N ; O=CO] | 608.1 | Reference Example 296 and 5- guanidinopentanoic acid |
| REF 418 | 4-[[3-[4- (cyanomethoxy)-2,3- difluoro- phenyl]imidazo[1,2- a]pyrazin-8- yl]amino]-N-[2- [[(2S)-2,5- diaminopentanoyl] amino]ethyl]-2-ethyl- benzamide formate | 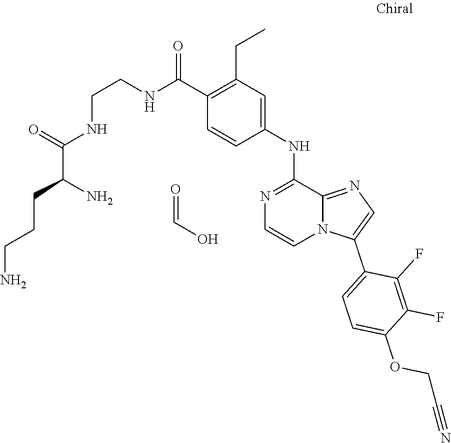 [SMILES: CCc1cc(Nc2nccn3c(-c4ccc(OCC#N)c(F)c4F)cnc23)ccc1C(=O)NCCNC(=O)[C@@H](N)CCCN ; O=CO] | 606.2 | Intermediate 116 and BOC-ORN(BOC)-OH |
| REF 419 | N-[2-(4- aminobutanoylamino) ethyl]-4-[[3-[4- (cyanomethoxy)-2,3- difluoro- phenyl]imidazo[1,2- a]pyrazin-8- yl]amino]-2-ethyl- benzamide formate | 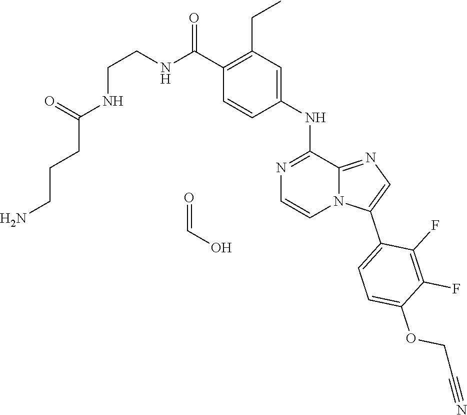 [SMILES: CCc1cc(Nc2nccn3c(-c4ccc(OCC#N)c(F)c4F)cnc23)ccc1C(=O)NCCNC(=O)CCCN ; O=CO] | 577.5 | Intermediate 116 and BOC-gamma-abu-OH |
| REF 420 | N-[2-[(4-amino-3- hydroxy- butanoyl)amino+ethyl]- 4-[[3-[4- (cyanomethoxy)-2,3- difluoro- phenyl]imidazo[1,2- a]pyrazin-8- yl]amino]-2-ethyl- benzamide formate | 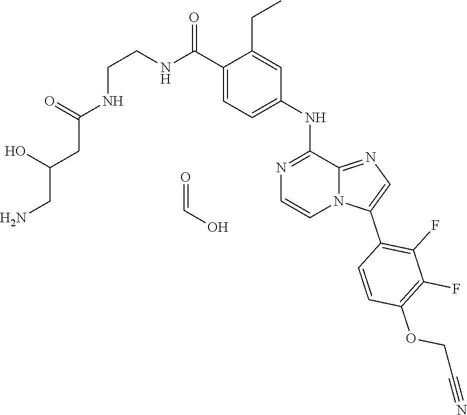 [SMILES: CCc1cc(Nc2nccn3c(-c4ccc(OCC#N)c(F)c4F)cnc23)ccc1C(=O)NCCNC(=O)CC(O)CN ; O=CO] | 593.4 | Intermediate 116 and 4- (tert- butoxycarbonylamino)- 3-hydroxy-butanoic acid |
| REF 421 | 4-[[3-[4- (cyanomethoxy)-2,3- difluoro- phenyl]imidazo[1,2- a]pyrazin-8- yl]amino]-N-[2- [[(2S)-2,6- diaminohexanoyl]amino] ethyl]-2-ethyl- benzamide formate | 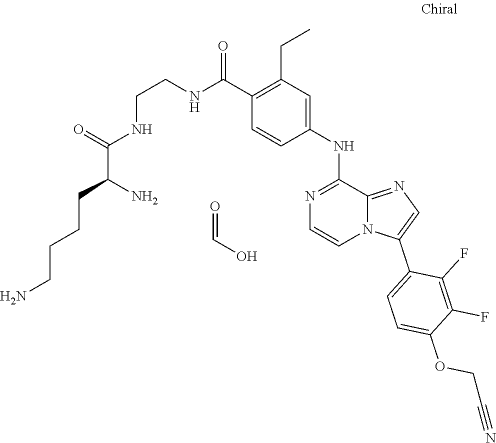 [SMILES: CCc1cc(Nc2nccn3c(-c4ccc(OCC#N)c(F)c4F)cnc23)ccc1C(=O)NCCNC(=O)[C@@H](N)CCCCN ; O=CO] | 620.4 | Intermediate 116 and BOC-LYS(BOC)-OH |
| REF 422 | 4-[[3-[4- (cyanomethoxy)-2,3- difluoro- phenyl]imidazo[1,2- a]pyrazin-8- yl]amino]-N-[2- [[(2S)-2,4- diaminobutanoyl]amino] ethyl]-2-ethyl- benzamide formate | 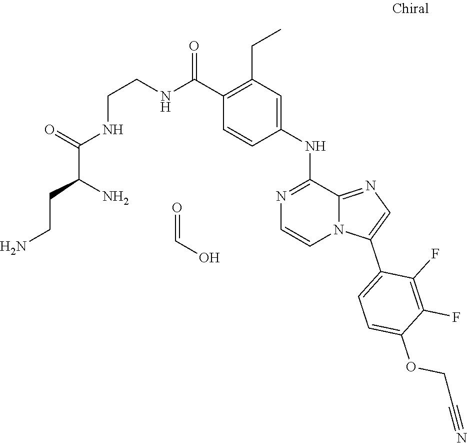 [SMILES: CCc1cc(Nc2nccn3c(-c4ccc(OCC#N)c(F)c4F)cnc23)ccc1C(=O)NCCNC(=O)[C@@H](N)CCN ; O=CO] | 592.4 | Intermediate 116 and (2S)-2,4-bis(tert- butoxycarbonylamino) butanoic acid |
| REF 423 | 4-[[3-[4- (cyanomethoxy)-2,3- difluoro- phenyl]imidazo[1,2- a]pyrazin-8- yl]amino]-N-[2- [[(2S)-2,3- diaminopropanoyl ] amino]ethyl]-2-ethyl- benzamide formate | 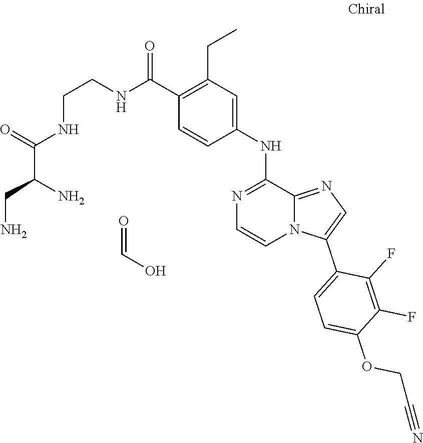 [SMILES: CCc1cc(Nc2nccn3c(-c4ccc(OCC#N)c(F)c4F)cnc23)ccc1C(=O)NCCNC(=O)[C@@H](N)CN ; O=CO] | 578.4 | Intermediate 116 and (2S)-2,3-bis(tert- butoxycarbonylamino) propanoic acid |
| REF 424 | N-[3-(3- aminopropanoylamino)- 2-hydroxy-propyl]- 4-[[3-(2,3-difluoro-4- methoxy- phenyl)imidazo[1,2- a]pyrazin-8- yl]amino]-2-ethyl- benzamide hydrochloride | 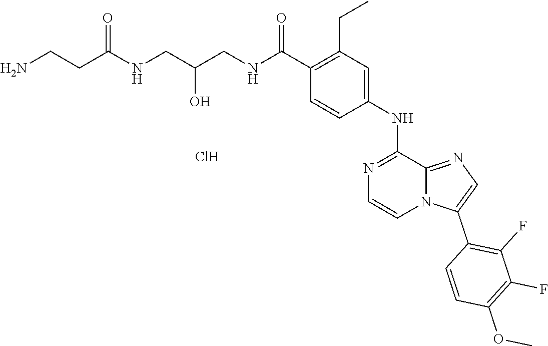 [SMILES: CCc1cc(Nc2nccn3c(-c4ccc(OC)c(F)c4F)cnc23)ccc1C(=O)NCC(O)CNC(=O)CCN ; Cl] | 568.3 | Intermediate 128 and 3- ((tert- butoxycarbonyl)amino) propanoic acid [CAS#3303-84-2] |
| REF 425 | N-[4-[[(2S)-2-amino- 5-guanidino- pentanoyl]amino] cyclohexyl]-4-[[3-(2,3- difluoro-4-methoxy- phenyl)imidazo[1,2- a]pyrazin-8- yl]amino]-2-ethyl- benzamide trifluoroacetate | 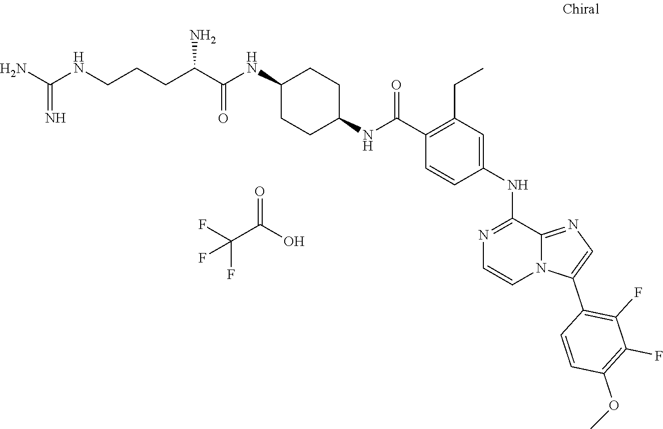 [SMILES: CCc1cc(Nc2nccn3c(-c4ccc(OC)c(F)c4F)cnc23)ccc1C(=O)N[C@H]1CC[C@@H](NC(=O)[C@@H](N)CCCNC(=N)N)CC1 ; O=C(O)C(F)(F)F] | 677.4 | Reference Example 426 and L-arginine. Deprotection with TFA/DCM 2/1 at rt for 2 h, then precipitated from the reaction mixture by the addition of diethylether. |
| REF 427 | N-[2-[[(2R)-2-amino- 5-guanidino- pentanoyl]amino]ethyl]- 4-[[3-[4- (cyanomethoxy)-2,3- difluoro- phenyl]imidazo[1,2- a]pyrazin-8- yl]amino]-2-ethyl- benzamide formate | 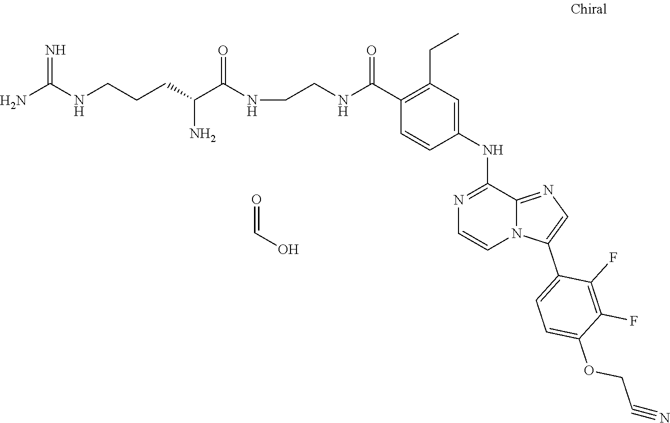 [SMILES: CCc1cc(Nc2nccn3c(-c4ccc(OCC#N)c(F)c4F)cnc23)ccc1C(=O)NCCNC(=O)[C@H](N)CCCNC(=N)N ; O=CO] | 648.1 | Intermediate 116 and FMOC-D-ARG-OH. Deprotection with 10 eq piperidine in DCM at rt fro 12h. Purification with prep HPLC (FA) |

### Intermediate 126

### (2S,4S)-1-tert-butoxycarbonyl-4-ethyl-4-hydroxy-pyrrolidine-2-carboxylic Acid

### And Intermediate 127

### (2S,4R)-1-tert-butoxycarbonyl-4-ethyl-4-hydroxy-pyrrolidine-2-carboxylic Acid

Ethylmagnesium bromide (7.27 mL, 21.81 mmol, 2.5 eq) was added dropwise to a THF (50 mL) solution of (2S)-1-tert-butoxycarbonyl-4-oxo-pyrrolidine-2-carboxylic acid [CAS #84348-37-8](2.0 g, 8.72 mmol, 1 eq) at −20° C. under nitrogen atmosphere. The resulting mixture was stirred at the same temperature for 1 h and then further stirred at 0° C. for 10 h. The reaction mixture was poured into 1 N aqueous hydrochloric acid solution (100 mL) under ice cooling, followed by extraction with ethyl acetate. The organic layer was washed with brine and dried over anhydrous Na₂SO₄. The solvent was evaporated under reduced pressure and the crude product was purified by prep. HPLC (FA as additive) to deliver (2S,4S)-1-tert-butoxycarbonyl-4-ethyl-4-hydroxy-pyrrolidine-2-carboxylic acid (Intermediate 126) (0.800 g, 3.09 mmol, 35.36% yield) as off white solid and (2S,4R)-1-tert-butoxycarbonyl-4-ethyl-4-hydroxy-pyrrolidine-2-carboxylic acid (Intermediate 127) (0.200 g, 0.770 mmol, 8.84% yield) as off white solid.

### Reference Example 428

### ((2S,3R,4S)-3,4-dihydroxypyrrolidin-2-yl)(4-(4-((3-(3-fluoro-4-methoxyphenyl)imidazo[1,2-a]pyrazin-8-yl)amino)-2-methylbenzoyl)piperazin-1-yl)methanone Hydrochloride

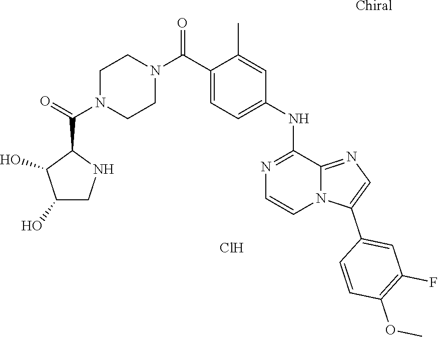 [SMILES: "Chiral" ; COc1ccc(-c2cnc3c(Nc4ccc(C(=O)N5CCN(C(=O)[C@H]6NC[C@H](O)[C@@H]6O)CC5)c(C)c4)nccn23)cc1F ; Cl]

### Step 1: tert-butyl (S)-2-(4-(4-((3-(3-fluoro-4-methoxyphenyl)imidazo[1,2-a]pyrazin-8-yl)amino)-2-methylbenzoyl)piperazine-1-carbonyl)-2,5-dihydro-1H-pyrrole-1-carboxylate

The title compound was prepared in analogy to Reference Example 339 from Reference Example 295 and (S)-1-(tert-butoxycarbonyl)-2,5-dihydro-1H-pyrrole-2-carboxylic acid without cleavage of the Boc-protective group.

MS (ESI) [M+H]⁺: 656.5

### Step 2: tert-butyl (2S,3R,4S)-2-(4-(4-((3-(3-fluoro-4-methoxyphenyl)imidazo[1,2-a]pyrazin-8-yl)amino)-2-methylbenzoyl)piperazine-1-carbonyl)-3,4-dihydroxypyrrolidine-1-carboxylate

tert-butyl (S)-2-(4-(4-((3-(3-fluoro-4-methoxyphenyl)imidazo[1,2-a]pyrazin-8-yl)amino)-2-methylbenzoyl)piperazine-1-carbonyl)-2,5-dihydro-1H-pyrrole-1-carboxylate (50 mg, 76.3 μmol, Eq: 1) was dissolved in a mixture of tert-BuOH (750 μl), tetrahydrofuran (200 μl) and water (50 μl). Osmium tetroxide in water (4%) (48.5 mg, 59.8 μL, 7.63 μmol, Eq: 0.1) was added, followed by 4-methyolmorpholine N-oxide (13.4 mg, 114 μmol, Eq: 1.5). The mixture was stirred overnight. Then additional osmium tetroxide in water (4%) (48.5 mg, 59.8 μl, 7.63 μmol, Eq: 0.1) and 4-methyolmorpholine N-oxide (13.4 mg, 114 μmol, Eq: 1.5) were added and the mixture was stirred over 72 h. The reaction was quenched by addition of sat. aq. Na₂S₂O₃ and then extracted with 2-MeTHF. The combined organic layers were washed with sat. aq. Na₂S₂O₃ and brine and then concentrated in vacuo. The residue was purified by prep. HPLC to obtain the title compound (52.6 mg) as a light brown solid.

MS (ESI) [M+H]⁺: 690.4

### Step 3: ((2S,3R,4S)-3,4-dihydroxypyrrolidin-2-yl)(4-(4-((3-(3-fluoro-4-methoxyphenyl)imidazo[1,2-a]pyrazin-8-yl)amino)-2-methylbenzoyl)piperazin-1-yl)methanone hydrochloride

4M HCl in dioxane (50 μl) was added to tert-butyl (2S,3R,4S)-2-(4-(4-((3-(3-fluoro-4-methoxyphenyl)imidazo[1,2-a]pyrazin-8-yl)amino)-2-methylbenzoyl)piperazine-1-carbonyl)-3,4-dihydroxypyrrolidine-1-carboxylate (8.3 mg, 12 μmol, Eq: 1) in DCM (200 μL). The reaction mixture was stirred overnight and then concentrated in vacuo to give the title compound (8.9 mg) as a white solid. MS (ESI) [M+H]⁺: 590.3

The following examples were prepared in analogy to Reference Example 428

| | | | MS | |
|---|---|---|---|---|
| | | | ESI | |
| Ex. | Name | Structure | [M + H]⁺ | Starting Material |
| REF 429 | (4-(4-((3-(2,3- difluoro-4- methoxy- phenyl)imida- zo[1,2-a]pyrazin-8- yl)amino)-2- methylbenzoyl)piper- azin-1- yl)((2S,3R,4S)-3,4- dihydroxypyrrolidin- 2-yl)methanone | 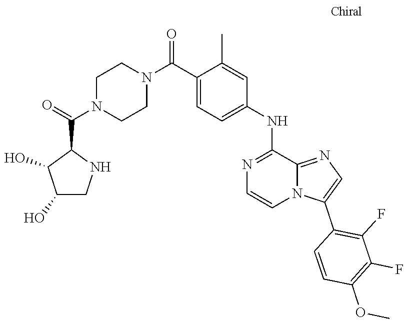 [SMILES: COc1ccc(-c2cnc3c(Nc4ccc(C(=O)N5CCN(C(=O)[C@H]6NC[C@H](O)[C@@H]6O)CC5)c(C)c4)nccn23)c(F)c1F] | 608.2 | from Reference Example 208 and (S)- 1-(tert- butoxycarbonyl)-2,5- dihydro-1H-pyrrole-2- carboxylic acid |
| REF 430 | (4-(4-((3-(2,3- difluoro-4-meth- oxyphenyl)imida- zo[1,2-a]pyrazin-8- yl)amino)-2- methylbenzoyl)piper- azin-1- yl((2R,3S,4R)-3,4- dihydroxypyrrolidin- 2-yl)methanone | 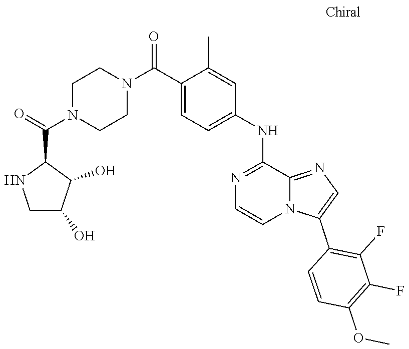 [SMILES: COc1ccc(-c2cnc3c(Nc4ccc(C(=O)N5CCN(C(=O)[C@@H]6NC[C@@H](O)[C@H]6O)CC5)c(C)c4)nccn23)c(F)c1F] | 608.3 | from Reference Example 208 and (R)- 1-(tert- butoxycarbonyl)-2,5- dihydro-1H-pyrrole-2- carboxylic acid |

### Reference Example 431

### Rel-(4-(4-((3-(2,3-difluoro-4-methoxyphenyl)imidazo[1,2-a]pyrazin-8-yl)amino)-2-methylbenzoyl)piperazin-1-yl)((3R,4S)-3,4-dihydroxypiperidin-3-yl)methanone

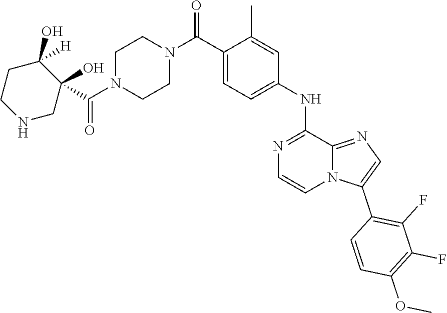 [SMILES: COc1ccc(-c2cnc3c(Nc4ccc(C(=O)N5CCN(C(=O)[C@]6(O)CNCC[C@H]6O)CC5)c(C)c4)nccn23)c(F)c1F]

### Step 1: 1-(tert-butoxycarbonyl)-1,2,5,6-tetrahydropyridine-3-carboxylic acid

1,2,5,6-tetrahydropyridine-3-carboxylic acid hydrochloride (1 g, 6.11 mmol, Eq: 1) was combined with dioxane (7.8 mL) and water (7.8 mL) to give a orange solution. Then di-tert-butyl dicarbonate (1.47 g, 6.72 mmol, Eq: 1.1) was slowly added as, a solution in dioxane (7.8 mL). After 15 min, NaOH (8 mL, 8 mmol, Eq: 1.31) was added and the RM stirred at RT overnight. The volatiles were removed, the reaction mixture was poured into 50 mL tBuOMe and extracted with 1 M HCl (2×25 mL). The aqueous layer was back-extracted with tBuOMe (2×25 mL). The organic layers were combined, washed with sat NaCl (2×25 mL), then dried over MgSO₄, filtered and concentrated in vacuo, the crude intermediate was used in the next step without further purification. MS (ESI) [M+H]⁺: 228.0

### Step 2: 1-tert-butyl 5-O-methyl 3,6-dihydro-2H-pyridine-1,5-dicarboxylate

1-(tert-Butoxycarbonyl)-1,2,5,6-tetrahydropyridine-3-carboxylic acid (409 mg, 1.8 mmol, Eq: 1) was dissolved in DMF (9 mL). potassium carbonate (298 mg, 2.16 mmol, Eq: 1.2) and Mel (511 mg, 225 μL, 3.6 mmol, Eq: 2) were successively added and RM was stirred at RT overnight. The RM was concentrated under HV. Residue was dissolved in ethyl acetate, filtered and concentrated under vacuum. MS (ESI) [M+H]⁺: 186.1 (carbamic acid, M-55)

### Step 3: 1-tert-butyl 3-O-methyl (3R,4S)-3,4-dihydroxypiperidine-1,3-dicarboxylate

1-(tert-butyl) 3-methyl 5,6-dihydropyridine-1,3(2H)-dicarboxylate (434 mg, 1.8 mmol, Eq: 1) was dissolved in tBuOH (20 mL). NMO (211 mg, 1.8 mmol, Eq: 1) and 4% osmium tetroxide in water (1.14 g, 1.41 mL, 180 μmol, Eq: 0.1) were successively added. Sodium thiosulfate (1.42 g, 8.99 mmol, Eq: 5) was added to quench the reaction but insoluble in tBuOH. The minimum amount of saturated Na₂S₂O₃ solution was added to solubilize the salt and RM was stirred for 1 h. RM was filtered through a pad of celite and concentrated under vacuum. Purification by combiflash. MS (ESI) [M+H]⁺: 176.1 (M-Boc)

### Step 4: 5-(tert-butyl) 3a-methyl (3aR,7aS)-2,2-dimethyldihydro-[1,3]dioxolo[4,5-c]pyridine-3a,5(4H,6H)-dicarboxylate

To a solution of rac-1-(tert-butyl) 3-methyl (3R,4S)-3,4-dihydroxypiperidine-1,3-dicarboxylate (320 mg, 1.16 mmol, Eq: 1) in DMF (1.16 mL) was added successively 2,2-dimethoxypropane (484 mg, 570 μl, 4.65 mmol, Eq: 4) and pTsOH (22.1 mg, 116 μmol, Eq: 0.1). RM was stirred and heated at 40° C. for 8 h and at 30° C. for 48 h. Purification by column chromatography, solid loaded with 1.2 g of silica, 12 g, heptane/ethyl acetate. Enantiomers were separated by SFC.

MS (ESI) [M+H]⁺: 260.2 (M-tBu)

### Step 5: (3aS,7aR)-5-(tert-butoxycarbonyl)-2,2-dimethyltetrahydro-[1,3]dioxolo[4,5-c]pyridine-3a(4H)-carboxylic Acid

To a solution of 5-(tert-butyl) 3a-methyl (3aS,7aR)-2,2-dimethyldihydro-[1,3]dioxolo[4,5-c]pyridine-3a,5(4H,6H)-dicarboxylate (100 mg, 317 μmol, Eq: 1) in THF (1 mL)/MeOH (500 μL) was added LiOH (1 mL, 2 mmol, Eq: 6.31). The RM was stirred at RT for 2 h. Volatiles were removed under vacuum and mixture was put in the freezer overnight. DCM was added and mixture was stirred. Aqueous phase was acidified with ammonium chloride and then with HCl 1M until pH 4. Phases were separated and extraction with 2×10 mL of DCM. Organic layers were combined, filtered through a pad of MgSO₄, concentrated in vacuo to provide an oil. MS (ESI) [M−H]⁻: 300.3

### Step 6: rel-(4-(4-((3-(2,3-difluoro-4-methoxyphenyl)imidazo[1,2-a]pyrazin-8-yl)amino)-2-methylbenzoyl)piperazin-1-yl)((3R,4S)-3,4-dihydroxypiperidin-3-yl)methanone

The title compound was prepared in analogy to Reference Example 339 from Reference Example 208 and rel-(3aR,7aS)-5-(tert-butoxycarbonyl)-2,2-dimethyltetrahydro-[1,3]dioxolo[4,5-c]pyridine-3a(4H)-carboxylic acid. MS (ESI) [M+H]⁺: 622.4

### Intermediate 124

### tert-butyl (3R)-3-[[tert-butyl(dimethyl)silyl]oxymethyl]piperazine-1-carboxylate

To a solution of tert-butyl (3R)-3-(hydroxymethyl)piperazine-1-carboxylate [CAS #278788-66-2] (200.0 mg, 0.920 mmol, 1 eq) and imidazole (75.54 mg, 1.11 mmol, 1.2 eq) in DCM (2 mL) was added tert-butyldimethylchlorosilane (153.31 mg, 1.02 mmol, 1.1 eq). The reaction was stirred at 20° C. for 12 h. The reaction was concentrated. The residue was purified by prep-TLC (PE:EtOAc=0:1) to give tert-butyl (3R)-3-[[tert-butyl(dimethyl)silyl]oxymethyl]piperazine-1-carboxylate (180 mg, 0.540 mmol, 58.89% yield) as colorless oil.

The following Intermediate was prepared in analogy to Intermediate 124

| Int. | Name | Starting Material |
|---|---|---|
| 125 | tert-butyl (3S)-3-[[tert- | tert-butyl (3S)-3- |
| | butyl(dimethyl)silyl]oxymethyl]piperazine- | (hydroxymethyl)piperazine- |
| | 1-carboxylate | 1-carboxylate |

### Intermediate 129

### 2-(4-(8-chloroimidazo[1,2-a]pyrazin-3-yl)-2,3-difluorophenoxy)propanenitrile

To a solution of Intermediate 54 (500 mg, 1.78 mmol) in MeCN (30 mL) was added potassium carbonate (491 mg, 3.55 mmol) and 3-bromobutanenitrile (263 mg, 1.78 mmol), the reaction was stirred for 16 h at 60° C. The reaction mixture was filtered and the filtrate was concentrated in vacuum. The residue was washed with brine and extracted in DCM. The organic layer was dried over anhydrous Na₂SO₄ and concentrated under reduced pressure to give 2-(4-(8-chloroimidazo[1,2-a]pyrazin-3-yl)-2,3-difluorophenoxy)propanenitrile (530 mg, 1.58 mmol, 89.2% yield) which was directly used for the next step without further purification.

### Intermediate 130

### 4-((3-(4-(1-Cyanoethoxy)-2,3-difluorophenyl)imidazo[1,2-a]pyrazin-8-yl)amino)-2-methylbenzoic Acid

To a solution of Intermediate 129 (520 mg, 1.55 mmol) in the mixture solvent of MeCN (20 mL) and acetic acid (4 mL) was added 4-amino-2-methylbenzoic acid (235 mg, 1.55 mmol), the reaction was stirred for 15 hours at 90° C. The reaction mixture was cooled to room temperature and filtered. The filter cake was dried in vacuum to give 4-((3-(4-(1-cyanoethoxy)-2,3-difluorophenyl)imidazo[1,2-a]pyrazin-8-yl)amino)-2-methylbenzoic acid (560 mg, 1.25 mmol, 80.2% yield). MS (ESI) [M+H]⁺: 450.1

### Intermediate 131

### 2-chloro-4-((3-(4-(1-cyanoethoxy)-2,3-difluorophenyl)imidazo[1,2-a]pyrazin-8-yl)amino)benzoic Acid

To a solution of Intermediate 129 (500 mg, 1.49 mmol) in the mixture solvent of MeCN (20 mL) and acetic acid (4 mL) was added 4-amino-2-chlorobenzoic acid (256 mg, 1.49 mmol), the reaction was stirred for 15 hours at 90° C. The reaction mixture was cooled to room temperature and filtered. The filter cake was dried in vacuum to give 2-chloro-4-((3-(4-(1-cyanoethoxy)-2,3-difluorophenyl)imidazo[1,2-a]pyrazin-8-yl)amino)benzoic acid (530 mg, 1.13 mmol, 75.5% yield). MS (ESI) [M+H]⁺: 470.3

### Example REF 432

### 2-Chloro-4-[[3-[4-(1-cyanoethoxy)-2,3-difluoro-phenyl]imidazo[1,2-a]pyrazin-8-yl]amino]-N-(2-piperazin-1-ylethyl)benzamide Formate

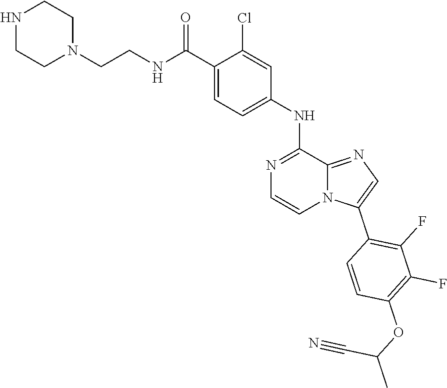 [SMILES: CC(C#N)Oc1ccc(-c2cnc3c(Nc4ccc(C(=O)NCCN5CCNCC5)c(Cl)c4)nccn23)c(F)c1F]

### Step 1: tert-butyl 4-[2-[[2-chloro-4-[[3-[4-(1-cyanoethoxy)-2,3-difluoro-phenyl]imidazo[1,2-a]pyrazin-8-yl]amino]benzoyl]amino]ethyl]piperazine-1-carboxylate

To a solution of Intermediate 131 (100 mg, 213 μmol) in DMF (3 mL) was added tert-butyl 4-(2-aminoethyl)piperazine-1-carboxylate (48.8 mg, 213 μmol), triethylamine (43.1 mg, 426 μmol) and 2,4,6-tripropyl-1,3,5,2,4,6-trioxatriphosphinane 2,4,6-trioxide (102 mg, 319 μmol). The reaction was stirred for 30 minutes at room temperature. The mixture was poured into water and filtered. The filter cake was dried in vacuum to give tert-butyl 4-[2-[[2-chloro-4-[[3-[4-(1-cyanoethoxy)-2,3-difluoro-phenyl]imidazo[1,2-a]pyrazin-8-yl]amino]benzoyl]amino]ethyl]piperazine-1-carboxylate (135 mg).

### Step 2: 2-chloro-4-[[3-[4-(1-cyanoethoxy)-2,3-difluoro-phenyl]imidazo[1,2-a]pyrazin-8-yl]amino]-N-(2-piperazin-1-ylethyl)benzamide Formate

To a solution of tert-butyl 4-(2-(2-chloro-4-((3-(4-(1-cyanoethoxy)-2,3-difluorophenyl)imidazo[1,2-a]pyrazin-8-yl)amino)benzamido)ethyl)piperazine-1-carboxylate (135 mg) in THF (10 mL) was added concentrated HCl (2 mL), the reaction was stirred for two hours at room temperature. The reaction mixture was cooled to 0° C. and basified with ammonia. The mixture was extracted in ethyl acetate and the organic layer was concentrated in vacuum. The residue was purified by preparative HPLC to give 2-chloro-4-[[3-[4-(1-cyanoethoxy)-2,3-difluoro-phenyl]imidazo[1,2-a]pyrazin-8-yl]amino]-N-(2-piperazin-1-ylethyl)benzamide (45 mg). MS obsd. (ESI⁺) [(M+H)⁺]: 581

The following examples were prepared in analogy to Example REF 432, the deprotection step 2 was only applied for intermediates derived from Boc-protected amines.

| | | | ESI | |
|---|---|---|---|---|
| Ex^{#} | Name | Structure | [M + H]⁺ | Starting Material |
| REF 433 | 2-[4-[8-[4-[4-[3- (dimethylamino)pro- pyl]piperazine-1- carbonyl]-3-methyl- anilino]imidazo[1,2- a]pyrazin-3-yl]-2,3- difluoro- phenoxy]propanenitrile formate | 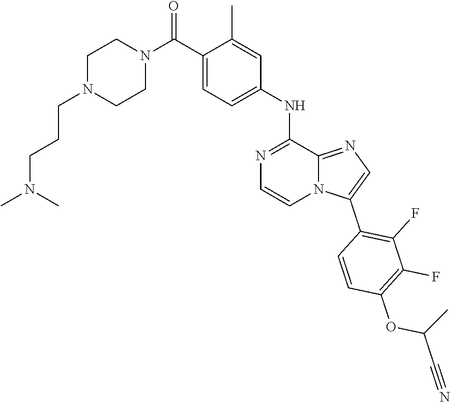 [SMILES: Cc1cc(Nc2nccn3c(-c4ccc(OC(C)C#N)c(F)c4F)cnc23)ccc1C(=O)N1CCN(CCCN(C)C)CC1] | 603.4 | Intermediate 130 and N,N-dimethyl-3- (piperazin-1- yl)propan-1-amine |
| REF 434 | 2-[4-[8-[4-[4-[2- (dimethylamino)ethyl] piperazine-1- carbonyl]-3-methyl- anilino]imidazo[1,2- a]pryazin-3-yl]-2,3- difluoro- phenoxy]propanenitrile; 2,2,2- trifluoroacetic acid | 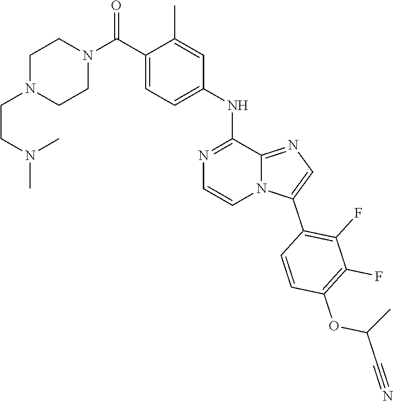 [SMILES: Cc1cc(Nc2nccn3c(-c4ccc(OC(C)C#N)c(F)c4F)cnc23)ccc1C(=O)N1CCN(CCN(C)C)CC1] | 589.4 | Intermediate 130 and N,N-dimethyl-2- (piperazin-1- yl)ethan-1-amine |
| 41 | 2-[4-[8-[3-chloro-4- [4-[2- (dimethylamino)ethyl] piperazine-1- carbonyl]anilino]imi- dazo[1,2-a]pyrazin- 3-yl]-2,3-difluoro- phenoxy]propanenitrile formate | 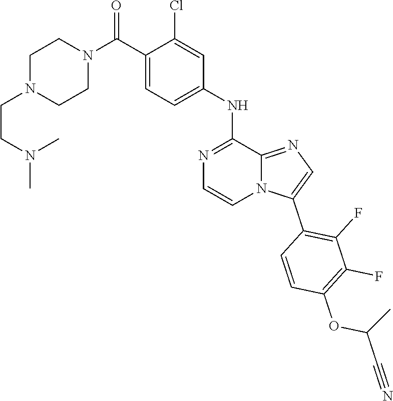 [SMILES: CC(C#N)Oc1ccc(-c2cnc3c(Nc4ccc(C(=O)N5CCN(CCN(C)C)CC5)c(Cl)c4)nccn23)c(F)c1F] | 609.5 | Intermediate 131 and N,N-dimethyl-2- (piperazin-1- yl)ethan-1-amine |
| 42 | 2-[4-[8-[3-chloro-4- [4-[3- (dimethylamino)pro- pyl]piperazine-1- carbonyl]anilino]imi- dazo[1,2-a]pyrazin- 3-yl]-2,3-difluoro- phenoxy]propanenitrile | 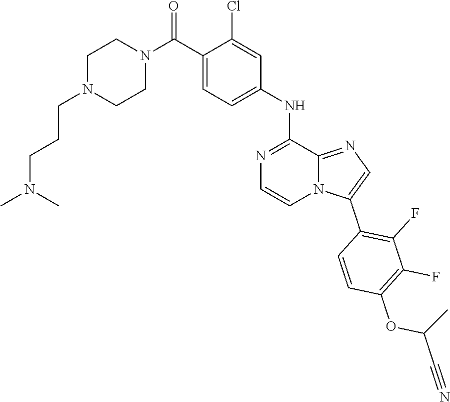 [SMILES: CC(C#N)Oc1ccc(-c2cnc3c(Nc4ccc(C(=O)N5CCN(CCCN(C)C)CC5)c(Cl)c4)nccn23)c(F)c1F] | 623.5 | Intermediate 131 and N,N-dimethyl-3- (piperazin-1- y)propan-1-amine |
| REF 435 | (2R)-2-[4-[8-[4-[4- [2- (dimethylamino)ethyl] piperazine-1- carbonyl]-3-methyl- anilino]imidazo[1,2- a]pryazin-3-yl]-2,3- difluoro- phenoxy]propanenitrile formate | 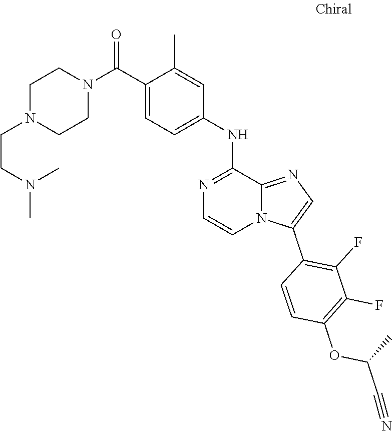 [SMILES: Cc1cc(Nc2nccn3c(-c4ccc(O[C@H](C)C#N)c(F)c4F)cnc23)ccc1C(=O)N1CCN(CCN(C)C)CC1] | 589.4 | The compound obtained by chiral separation of Reference Example 434 |
| REF 436 | ((2S)-2-[4-[8-[4-[4- [2- (dimethylamino)ethyl] piperazine-1- carbonyl]-3-methyl- anilino]imidazo[1,2- a]pyrazin-3-yl]-2,3- difluoro- phenoxy]propanenitrile formate | 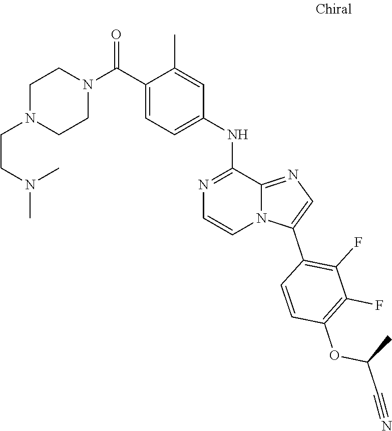 [SMILES: Cc1cc(Nc2nccn3c(-c4ccc(O[C@@H](C)C#N)c(F)c4F)cnc23)ccc1C(=O)N1CCN(CCN(C)C)CC1] | 589.4 | The compound was obtained by chiral separation of Reference Example 434 |

### Reference Example 437

### 3-(4-(2-(4-((3-(4-(difluoromethoxy)phenyl)imidazo[1,2-a]pyrazin-8-yl)amino)-N,2-dimethylbenzamido)ethyl)piperidin-1-yl)propanoic Acid

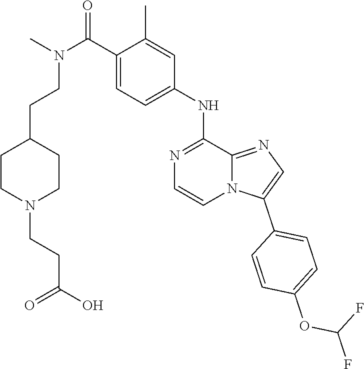 [SMILES: Cc1cc(Nc2nccn3c(-c4ccc(OC(F)F)cc4)cnc23)ccc1C(=O)N(C)CCC1CCN(CCC(=O)O)CC1]

### Step 1)

### N-[2-[1-(2-cyanoethyl)-4-piperidyl]ethyl]-4-[[3-[4-(difluoromethoxy)phenyl]imidazo[1,2-a]pyrazin-8-yl]amino]-N,2-dimethyl-benzamide

4-((3-(4-(difluoromethoxy)phenyl)imidazo[1,2-a]pyrazin-8-yl)amino)-N,2-dimethyl-N-(2-(piperidin-4-yl)ethyl)benzamide (Reference Example 174, 96 mg, 180 μmol, Eq: 1), acrylonitrile (95.3 mg, 1.8 mmol, Eq: 10) and DIPEA (116 mg, 157 μL, 898 μmol, Eq: 5) were combined with dioxane (3 mL) and stirred at 100° C. overnight. The reaction mixture was concentrated to dryness and purified by flash chromatography to give a brown viscous oil (53 mg, yield: 54%). MS (ESI): [M+H]⁺: 588.5

### Step 2)

### 3-[4-[2-[[4-[[3-[4-(difluoromethoxy)phenyl]imidazo[1,2-a]pyrazin-8-yl]amino]-2-methyl-benzoyl]-methyl-amino]ethyl]-1-piperidyl]propanoic Acid

N-(2-(1-(2-cyanoethyl)piperidin-4-yl)ethyl)-4-((3-(4-(difluoromethoxy)phenyl)imidazo[1,2-a]pyrazin-8-yl)amino)-N,2-dimethylbenzamide (25 mg, 42.5 μmol, Eq: 1) was combined with dioxane (0.25 mL). 2M aq NaOH (425 μL, 851 μmol, Eq: 20) was added, and the reaction mixture was stirred at 100° C. overnight. After cooling down to RT, the reaction mixture was directly acidified with 2M aq HCl solution. The crude product obtained was purified by preparative HPLC. Finally the product was lyophilized to give the target compound as a light brown solid (3.7 mg, yield: 14%). MS (ESI): [M−H]⁻: 605.8

### Intermediate 132

### [4-(2-chloroethyl)piperazin-1-yl]-[2-chloro-4-[[3-(3-fluoro-4-methoxy-phenyl)imidazo[1,2-a]pyrazin-8-yl]amino]phenyl]methanone

2-chloro-4-((3-(3-fluoro-4-methoxyphenyl)imidazo[1,2-a]pyrazin-8-yl)amino)benzoic acid (Intermediate 2) (200 mg, 484 μmol, Eq: 1) was combined with DMF (6 mL). DIPEA (188 mg, 254 μl, 1.45 mmol, Eq: 3) and HATU (368 mg, 969 μmol, Eq: 2.00) were added, followed, after stirring at RT for 15 minutes, by addition of 1-(2-chloroethyl)piperazine [CAS 61308-25-6](108 mg, 727 μmol, Eq: 1.5). After stirring for 3 h at RT, the reaction mixture was poured into 25 mL H₂O and extracted with ethyl acetate. The crude product was used without further purification. MS (ESI): [M+H]⁺: 544.3

### Example 43

### (2-Chloro-4-((3-(3-fluoro-4-methoxyphenyl)imidazo[1,2-a]pyrazin-8-yl)amino)phenyl)(4-(2-((2-hydroxyethyl)amino)ethyl)piperazin-1-yl)methanone

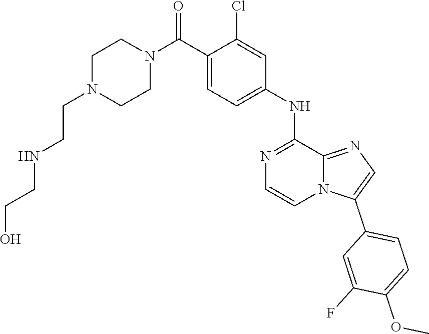 [SMILES: COc1ccc(-c2cnc3c(Nc4ccc(C(=O)N5CCN(CCNCCO)CC5)c(Cl)c4)nccn23)cc1F]

(2-chloro-4-((3-(3-fluoro-4-methoxyphenyl)imidazo[1,2-a]pyrazin-8-yl)amino)phenyl)(4-(2-chloroethyl)piperazin-1-yl)methanone (Intermediate 132) (40 mg, 73.6 μmol, Eq: 1), 2-aminoethane-1-ol (6.72 mg, 110 μmol, Eq: 1.50), sodium carbonate (11.7 mg, 110 μmol, Eq: 1.50), potassium iodide (611 μg, 3.68 μmol, Eq: 0.05) were combined with BuOH (800 μl) and stirred at 105° C. for 24 h. After extraction with DCM/water, the crude material was purified via prep HPLC to give the target compound (6.9 mg, yield: 16%). MS (ESI): [M+H]⁺: 568.2

### Intermediate 48

### Methyl 4-amino-2-vinylbenzoate

A mixture of methyl 4-amino-2-bromobenzoate (500 mg, 2.17 mmol, Eq: 1), 4,4,5,5-tetramethyl-2-vinyl-1,3,2-dioxaborolane (502 mg, 553 μL, 3.26 mmol, Eq: 1.5), Na₂CO₃ (461 mg, 4.35 mmol, Eq: 2) and 1,1′-bis(diphenylphosphino)ferrocenedichloro palladium(II) dichloromethane complex (159 mg, 217 μmol, Eq: 0.1) in dioxane (6 mL) and water (600 μL) was heated in a microwave at 100° C. for 30 min. The reaction mixture was then poured into 30 mL H₂O and extracted with ethyl acetate (3×50 mL). The organic layers were dried over MgSO₄ and concentrated in vacuo. The crude material was purified by flash chromatography. MS (ESI): [M+H]⁺: 178.2

### Reference Example 438

### (E)-4-((3-(4-(Difluoromethoxy)phenyl)imidazo[1,2-a]pyrazin-8-yl)amino)-N-methyl-2-(prop-1-en-1-yl)benzamide

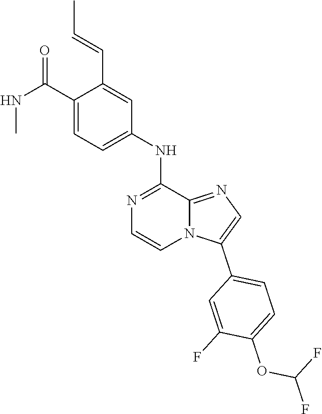 [SMILES: C/C=C/c1cc(Nc2nccn3c(-c4ccc(OC(F)F)c(F)c4)cnc23)ccc1C(=O)NC]

2-Bromo-4-((3-(4-(difluoromethoxy)phenyl)imidazo[1,2-a]pyrazin-8-yl)amino)-N-methylbenzamide (Intermediate 83) (50 mg, 102 μmol, Eq: 1), (E)-prop-1-en-1-ylboronic acid (13.2 mg, 154 μmol, Eq: 1.5), Na₂CO₃ (21.7 mg, 205 μmol, Eq: 2) and 1,1′-bis(diphenylphosphino)ferrocenedichloro palladium(II) dichloromethane complex (7.49 mg, 10.2 μmol, Eq: 0.1) were combined in dioxane (1.5 mL) and water (150 μL) and heated in the microwave at 90° C. for 30 min. The reaction mixture was poured into 50 mL H₂O and extracted with ethyl acetate (3×75 mL). The organic layers were dried over MgSO₄ and concentrated in vacuo. The crude material was purified by prep HPLC to give the target compound (68%). MS (ESI): [M+H]⁺: 450.2

### Reference Example 439

### 4-((3-(4-(Difluoromethoxy)phenyl)imidazo[1,2-a]pyrazin-8-yl)amino)-N-methyl-2-propylbenzamide

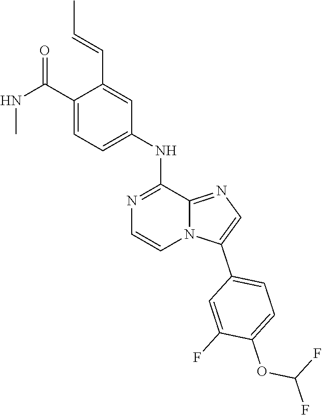 [SMILES: C/C=C/c1cc(Nc2nccn3c(-c4ccc(OC(F)F)c(F)c4)cnc23)ccc1C(=O)NC]

4-((3-(4-(Difluoromethoxy)phenyl)imidazo[1,2-a]pyrazin-8-yl)amino)-N-methyl-2-propylbenzamide (Reference Example 438) (20.8 mg, 46.1 μmol, 76.7% yield) was dissolved in MeOH and palladium on carbon was added. The reaction was stirred under hydrogen. The reaction mixture was carefully filtered under argon through Celite. MS (ESI): [M+H]⁺: 452.1

### Intermediate 133

### N-(2,2-Diethoxyethyl)-4-((3-(4-methoxyphenyl)imidazo[1,2-a]pyrazin-8-yl)amino)-N,2-dimethylbenzamide

4-((3-(4-methoxyphenyl)imidazo[1,2-a]pyrazin-8-yl)amino)-2-methylbenzoic acid (Intermediate 4) (150 mg, 401 μmol, Eq: 1) and 2,2-diethoxy-N-methylethanamine (70.8 mg, 481 μmol, Eq: 1.20) were combined with DMF (4.5 mL). HATU (305 mg, 801 μmol, Eq: 2.00) and DIPEA (155 mg, 210 μl, 1.2 mmol, Eq: 3.00) were added, and the reaction mixture was stirred at RT. The reaction mixture was directly purified by flash chromatography (reverse phase, 20 g, 0% to 100% acetonitrile in water). MS (ESI): [M+H]⁺: 504.3

### Reference Example 440

### N-((5-Amino-1,3-dioxan-2-yl)methyl)-4-((3-(4-methoxyphenyl)imidazo[1,2-a]pyrazin-8-yl)amino-N,2-dimethylbenzamide

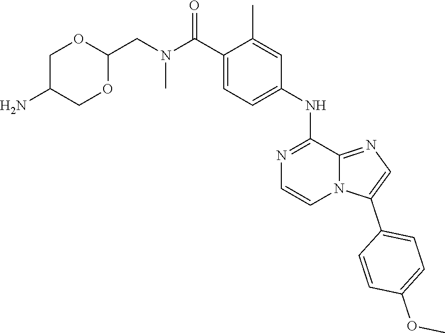 [SMILES: COc1ccc(-c2cnc3c(Nc4ccc(C(=O)N(C)CC5OCC(N)CO5)c(C)c4)nccn23)cc1]

N-(2,2-Diethoxyethyl)-4-((3-(4-methoxyphenyl)imidazo[1,2-a]pyrazin-8-yl)amino)-N,2-dimethylbenzamide (Intermediate 133) (100 mg, 199 μmol, Eq: 1) and benzyl (1,3-dihydroxypropan-2-yl)carbamate (47 mg, 209 μmol, Eq: 1.05) were combined with toluene (1.5 mL), pTsOH (1.89 mg, 9.93 μmol, Eq: 0.05) was added, and the reaction mixture was stirred at reflux overnight. After cooling down to RT, the reaction mixture was concentrated to dryness, and purified by flash chromatography. MS (ESI): [M+H]⁺: 637.4.

The product obtained was dissolved in MeOH, palladium on carbon 10% was added and the reaction mixture obtained was stirred under hydrogen. The crude was purified by flash chromatography. MS (ESI): [M+H]⁺: 503.3

The following examples were prepared in analogy to Example REF 440

| | | | MS | |
|---|---|---|---|---|
| | | | ESI | |
| Ex. | Name | Structure | [M + H]⁺ | Starting Material |
| REF 441 | N-(2-(aminomethyl)- 1,3-dioxan-5-yl)-4- ((3-(4- methoxyphenyl)imidazo [1,2-a]pyrazin-8- yl)amino)-2- methylbenzamide | 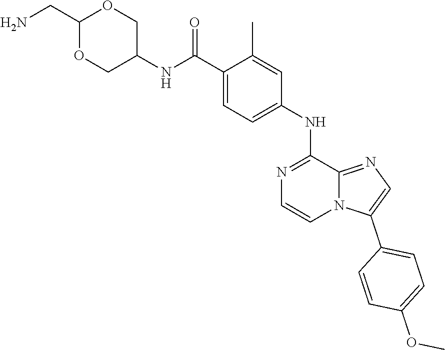 [SMILES: COc1ccc(-c2cnc3c(Nc4ccc(C(=O)NC5COC(CN)OC5)c(C)c4)nccn23)cc1] | 489.2 | Intermediate 134 and benzyl (2,2- diethoxyethyl)carbamate |

### Intermediate 134

### N-(1,3-Dihydroxypropan-2-yl)-4-((3-(4-methoxyphenyl)imidazo[1,2-a]pyrazin-8-yl)amino)-2-methylbenzamide

4-((3-(4-Methoxyphenyl)imidazo[1,2-a]pyrazin-8-yl)amino)-2-methylbenzoic acid (Intermediate 4) (150 mg, 401 μmol, Eq: 1) and 2-aminopropane-1,3-diol (36.5 mg, 401 μmol, Eq: 1.50) were combined with DMF (3 mL) to give a light yellow suspension. HATU (152 mg, 401 μmol, Eq: 1.50) and DIPEA (104 mg, 140 μl, 801 μmol, Eq: 3.00) were added, and the reaction mixture was stirred at RT. The reaction mixture was directly purified by flash chromatography (reverse phase, 20 g, 0% to 100% acetonitrile in water). MS (ESI): [M+H]⁺: 448.2.

### Reference Example 442

### 1-(2-Ethyl-4-((3-(3-fluoro-4-methoxyphenyl)imidazo[1,2-a]pyrazin-8-yl)amino)benzoyl)piperidine-4-carboxylic Acid

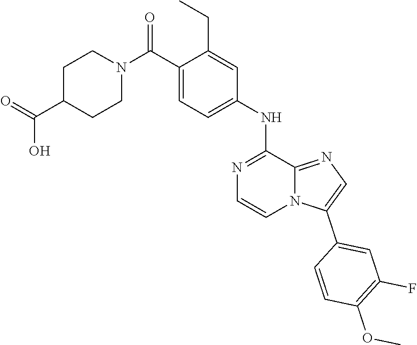 [SMILES: CCc1cc(Nc2nccn3c(-c4ccc(OC)c(F)c4)cnc23)ccc1C(=O)N1CCC(C(=O)O)CC1]

This example was prepared in analogy to Reference Example 1 from Intermediate 106. MS (ESI): [M+H]⁺: 518.3

### Reference Example 443

### N-(2-((2-Aminoethyl)sulfonyl)ethyl)-4-((3-(4-(difluoromethoxy)phenyl)imidazo[1,2-a]pyrazin-8-yl)amino)-2-methylbenzamide

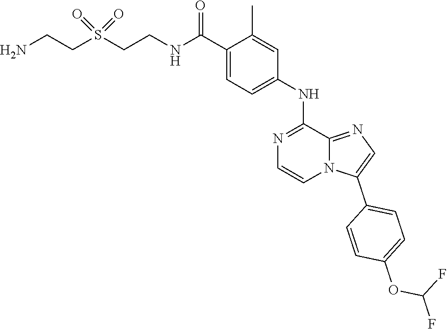 [SMILES: Cc1cc(Nc2nccn3c(-c4ccc(OC(F)F)cc4)cnc23)ccc1C(=O)NCCS(=O)(=O)CCN]

A solution of 3-chloroperoxybenzoic acid (19.9 mg, 80.7 μmol, Eq: 2.2) in DCM (2 mL) was added slowly to a solution of N-(2-((2-aminoethyl)thio)ethyl)-4-((3-(4-(difluoromethoxy)phenyl)imidazo[1,2-a]pyrazin-8-yl)amino)-2-methylbenzamide (Intermediate 84) (18.8 mg, 36.7 μmol, Eq: 1) in DCM (2 mL) at −78° C. under Ar. The mixture was stirred at −78° C. for 1 h and then warmed to RT. The reaction mixture was poured into 5 mL 1 M NaOH and extracted with DCM (5×20 mL). The organic layers were dried over sodium sulphate and concentrated in vacuo. MS (ESI): [M+H]⁺: 545.2

### Reference Example 444

### N-(2-((2-Aminoethyl)sulfinyl)ethyl)-4-((3-(4-(difluoromethoxy)phenyl)imidazo[1,2-a]pyrazin-8-yl)amino)-2-methylbenzamide

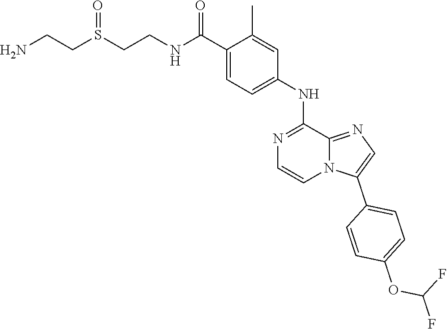 [SMILES: Cc1cc(Nc2nccn3c(-c4ccc(OC(F)F)cc4)cnc23)ccc1C(=O)NCCS(=O)CCN]

A solution of 3-chloroperoxybenzoic acid (7.4 mg, 33 μmol, Eq: 0.9) in DCM (2 mL) was added slowly to a solution of N-(2-((2-aminoethyl)thio)ethyl)-4-((3-(4-(difluoromethoxy)phenyl)imidazo[1,2-a]pyrazin-8-yl)amino)-2-methylbenzamide (84-001) (18.8 mg, 36.7 μmol, Eq: 1) in DCM (2 mL) at −78° C. under Ar. The mixture was stirred at −78° C. for 1 h. The RM was quenched at −78° C. with NaOH. The reaction mixture was poured into 5 mL 1 M NaOH and extracted with DCM (3×20 mL). The organic layers were dried over Na₂SO₄ and concentrated in vacuo. MS (ESI): [M+H]⁺: 529.2

### Intermediate 107

### 2-(4-(Difluoromethoxy)-2,3-difluorophenyl)-4,4,5,5-tetramethyl-1,3,2-dioxaborolane

### Step 1: 1-bromo-4-(difluoromethoxy)-2,3-difluorobenzene

4-bromo-2,3-difluorophenol (500 mg, 2.39 mmol, Eq: 1), sodium 2-chloro-2,2-difluoroacetate (730 mg, 4.78 mmol, Eq: 2) and potassium carbonate (397 mg, 2.87 mmol, Eq: 1.2) were dissolved in DMF (6 mL) and water (1.5 mL). The reaction mixture was heated to 100° C. and stirred for 3 h. The reaction mixture was poured into 20 mL sat NaHCO₃ and extracted with DCM (5×40 mL). The organic layers were dried over Na₂SO₄ and concentrated in vacuo. The crude material was purified by flash chromatography.

### Step 2: 2-(4-(difluoromethoxy)-2,3-difluorophenyl)-4,4,5,5-tetramethyl-1,3,2-dioxaborolane

1-bromo-4-(difluoromethoxy)-2,3-difluorobenzene (240 mg, 927 μmol, Eq: 1), 4,4,4′,4′,5,5,5′,5′-octamethyl-2,2′-bi(1,3,2-dioxaborolane) (282 mg, 1.11 mmol, Eq: 1.2), potassium acetate (182 mg, 1.85 mmol, Eq: 2) and dichlorobis(triphenylphosphine)palladium(II) (32.5 mg, 46.3 μmol, Eq: 0.05) were dissolved in dioxane (1 mL). The reaction mixture was heated to 100° C. and stirred for O/N. The crude material was purified by flash chromatography (silica gel, 40 g, 0% to 35% ethyl acetate in heptane). MS (ESI): [M+H]⁺: 306.1

### Intermediate 88

### 2-(2-chloro-4-(4,4,5,5-tetramethyl-1,3,2-dioxaborolan-2-yl)phenoxy)acetonitrile

2-(4-bromo-2-chlorophenoxy)acetonitrile (500 mg, 2.03 mmol, Eq: 1), 4,4,4′,4′,5,5,5′,5′-octamethyl-2,2′-bi(1,3,2-dioxaborolane) (567 mg, 2.23 mmol, Eq: 1.10), potassium acetate (398 mg, 4.06 mmol, Eq: 2.00) and bis(triphenylphosphine)palladium(II)chloride (71.2 mg, 101 μmol, Eq: 0.05) were combined with dioxane (7.5 mL). After degassing with N₂, the reaction mixture was heated to 100° C. and stirred overnight. The reaction mixture was cooled to RT, adsorbed on Isolute HM-N and after evaporation to dryness, the crude material was purified by flash chromatography (silica gel, 40 g, 0% to 80% ethyl acetate in heptane). MS (ESI): [M+H]⁺: 293.9

### Reference Example 445

### (2-(4-((3-(2,3-difluoro-4-methoxyphenyl)imidazo[1,2-a]pyrazin-8-yl)amino)-2-ethylbenzamido)ethyl)glycine compound with 2,2,2-trifluoroacetic Acid

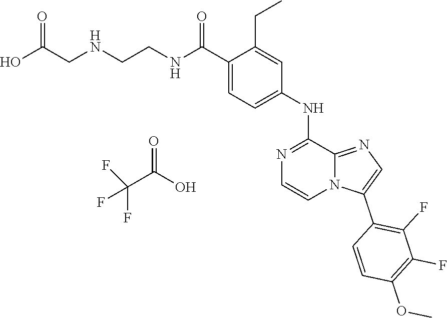 [SMILES: CCc1cc(Nc2nccn3c(-c4ccc(OC)c(F)c4F)cnc23)ccc1C(=O)NCCNCC(=O)O ; O=C(O)C(F)(F)F]

### Step 1: tert-butyl (2-(4-((3-(2,3-difluoro-4-methoxyphenyl)imidazo[1,2-a]pyrazin-8-yl)amino)-2-ethylbenzamido)ethyl)glycinate

N-(2-aminoethyl)-4-((3-(2,3-difluoro-4-methoxyphenyl)imidazo[1,2-a]pyrazin-8-yl)amino)-2-ethylbenzamide hydrochloride (Intermediate REF 296) (40 mg, 79.5 μmol, Eq: 1) was combined with DMF (600 μl). TEA (32.2 mg, 44.3 μl, 318 μmol, Eq: 4.00) was added dropwise, followed by addition of tert-butyl 2-chloroacetate (13.2 mg, 12.5 μL, 87.5 μmol, Eq: 1.10). The reaction mixture was stirred at RT for 24 h. The crude material was purified by prep HPLC. MS (ESI): [M+H]⁺: 581.4

### Step 2: 2 (2-(4-((3-(2,3-difluoro-4-methoxyphenyl)imidazo[1,2-a]pyrazin-8-yl)amino)-2-ethylbenzamido)ethyl)glycine Compound Trifluoroacetate

tert-Butyl (2-(4-((3-(2,3-difluoro-4-methoxyphenyl)imidazo[1,2-a]pyrazin-8-yl)amino)-2-ethylbenzamido)ethyl)glycinate (15 mg, 25.8 μmol, Eq: 1) was combined with DCM (200 μL). TFA (29.5 mg, 19.9 μL, 258 μmol, Eq: 10.0) was added, and the reaction mixture was stirred at RT. The reaction mixture was concentrated to dryness, and lyophilized.

MS (ESI): [M+H]⁺: 525.2

The following examples were prepared in analogy to Reference Example 445

| | | | MS | |
|---|---|---|---|---|
| | | | ESI | |
| Ex# | Name | Structure | [M + H]⁺ | Starting Material |
| REF 446 | (3-(4-((3-(2,3- difluoro-4- methoxyphenyl)imi- dazo[1,2-a]pyrazin- 8-yl)amino)-2- ethylbenzami- do)propyl)glycine compound trifluoroacetate | 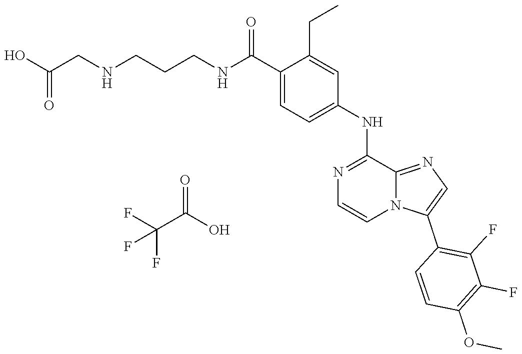 [SMILES: CCc1cc(Nc2nccn3c(-c4ccc(OC)c(F)c4F)cnc23)ccc1C(=O)NCCCNCC(=O)O ; O=C(O)C(F)(F)F] | 539.2 | From Intermediate 87 and tert-butyl 2- chloroacetate |
| REF 447 | (5-(4-((3-(2,3- difluoro-4-meth- oxyphenyl)imi- dazo[1,2-a]pyrazin- 8-yl)amino)-2- entylbenzami- do)pentyl)glycine hydrochloride | 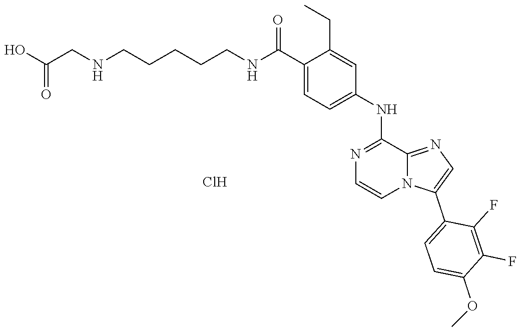 [SMILES: CCc1cc(Nc2nccn3c(-c4ccc(OC)c(F)c4F)cnc23)ccc1C(=O)NCCCCCNCC(=O)O ; Cl] | 567.2 | From Intermediate REF 298 and tert-butyl 2-chloroacetate |

### Intermediate 135

### N-(2-(2-chloroethoxy)ethyl)-4-((3-(4-(difluoromethoxy)-2,3-difluorophenyl)imidazo[1,2-a]pyrazin-8-yl)amino)-2-ethylbenzamide

To a solution of 4-((3-(4-(difluoromethoxy)-2,3-difluorophenyl)imidazo[1,2-a]pyrazin-8-yl)amino)-2-ethylbenzoic acid (75 mg, 163 μmol, Eq: 1) in DMF (815 μl) was added DTPEA (84.2 mg, 114 μl, 652 μmol, Eq: 4) and HATU (124 mg, 326 μmol, Eq: 2). RM was stirred for 15 min. Then 2-(2-chloroethoxy)ethan-1-amine hydrochloride (26.1 mg, 163 μmol, Eq: 1) was added and the RM was stirred overnight. The RM was purified via prep HPLC. MS (ESI): [M+H]⁺:566.3

### The following intermediates were prepared in analogy to Intermediate 135

| | | MS ESI | |
|---|---|---|---|
| Int. | Name | [M + H]⁺ | Starting Material |
| 136 | N-(2-(2-chloroethoxy)ethyl)-4-((3-(2,3-difluoro- | 530.2 | From 86 and 2-(2- |
| | 4-methoxyphenyl)imidazo[1,2-a]pyrazin-8- | | chloroethoxy)ethan-1- |
| | yl)amino)-2-ethylbenzamide | | amine hydrochloride |

### Reference Example 448

### N-(2-(2-(4-((3-(4-(Difluoromethoxy)-2,3-difluorophenyl)imidazo[1,2-a]pyrazin-8-yl)amino)-2-ethylbenzamido)ethoxy)ethyl)-N-methylglycine Trifluoroacetate

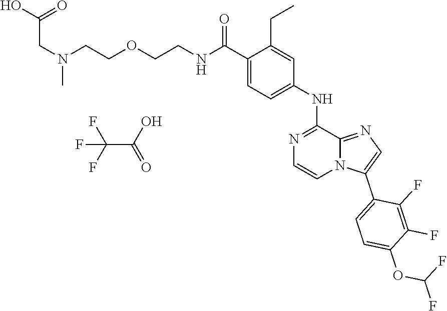 [SMILES: CCc1cc(Nc2nccn3c(-c4ccc(OC(F)F)c(F)c4F)cnc23)ccc1C(=O)NCCOCCN(C)CC(=O)O ; O=C(O)C(F)(F)F]

### Step 1: tert-Butyl N-(2-(2-(4-((3-(4-(difluoromethoxy)-2,3-difluorophenyl)imidazo[1,2-a]pyrazin-8-yl)amino)-2-ethylbenzamido)ethoxy)ethyl)-N-methylglycinate

A mixture of N-(2-(2-chloroethoxy)ethyl)-4-((3-(4-(difluoromethoxy)-2,3-difluorophenyl)imidazo[1,2-a]pyrazin-8-yl)amino)-2-ethylbenzamide (Intermediate 135) (50 mg, 88.3 μmol, Eq: 1), tert-butyl methylglycinate (25.7 mg, 177 μmol, Eq: 2), K₂CO₃ (24.4 mg, 177 μmol, Eq: 2) and KI (14.7 mg, 88.3 μmol, Eq: 1) in MeCN/dioxane was heated at 90° C. for 2 days. Purification by flash chromatography. MS (ESI): [M+H]⁺: 675.4

### Step 2: N-(2-(2-(4-((3-(4-(difluoromethoxy)-2,3-difluorophenyl)imidazo[1,2-a]pyrazin-8-yl)amino)-2-ethylbenzamido)ethoxy)ethyl)-N-methylglycine compound Trifluoroacetate

2,2,2-Trifluoroacetic acid (298 mg, 200 μL, 2.61 mmol, Eq: 36.7) was added to a solution of tert-butyl N-(2-(2-(4-((3-(4-(difluoromethoxy)-2,3-difluorophenyl)imidazo[1,2-a]pyrazin-8-yl)amino)-2-ethylbenzamido)ethoxy)ethyl)-N-methylglycinate (48 mg, 71.1 μmol, Eq: 1) in DCM (200 μL). RM was stirred for 24 h. The reaction mixture was concentrated to dryness, and lyophilized. MS (ESI): [M+H]⁺: 619.4

The following examples were prepared in analogy to Reference Example 448

| | | | MS | |
|---|---|---|---|---|
| | | | ESI | Starting |
| Ex # | Name | Structure | [M + H]⁺ | Material |
| REF 449 | N-(2-(2-(4-((3- (2,3-difluoro-4- methoxyphenyl) imidazo[1,2- a]pyrazin- 8-yl)amino)-2- ethyl- benzamido) ethoxy) ethyl)-N- methylglycine hydrochloride | 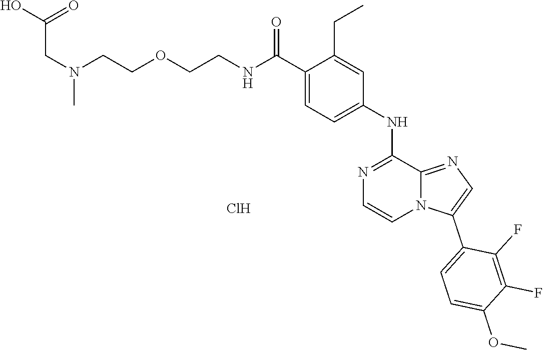 [SMILES: CCc1cc(Nc2nccn3c(-c4ccc(OC)c(F)c4F)cnc23)ccc1C(=O)NCCOCCN(C)CC(=O)O ; Cl] | 583.3 | From Intermediate 136 and tert-butyl methyl- glycinate. Deprotection with HCl |

### Reference Example 450

### 1-(4-((3-(4-(cyanomethoxy)-2,3-difluorophenyl)imidazo[1,2-a]pyrazin-8-yl)amino)-2-methylbenzoyl)-N-(3-(methylamino)propyl)piperidine-4-carboxamide

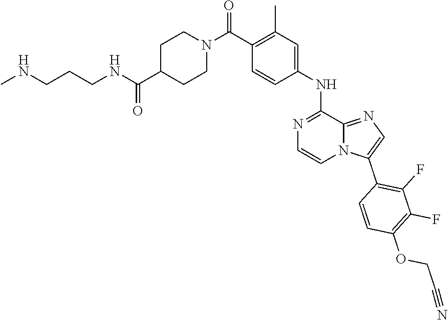 [SMILES: CNCCCNC(=O)C1CCN(C(=O)c2ccc(Nc3nccn4c(-c5ccc(OCC#N)c(F)c5F)cnc34)cc2C)CC1]

### Step 1: benzyl 4-((3-((tert-butoxycarbonyl)(methyl)amino)propyl)carbamoyl)piperidine-1-carboxylate

To a stirred solution of 1-[(benzyloxy)carbonyl]piperidine-4-carboxylic acid (500.0 mg, 1.9 mmol, 1 eq) and N-(3-aminopropyl)-N-methylcarbamic acid tert-butyl ester (357.53 mg, 1.9 mmol, 1 eq) in DMF (5 mL) was added O-(7-azabenzotriazol-1-yl)-N,N,N′,N′-tetramethyluronium hexafluorophosphate (HATU) (1444.15 mg, 3.8 mmol, 2 eq) and N,N-diisopropylethylamine (1.32 mL, 7.6 mmol, 4 eq) in 20° C. and the mixture stirred at 20° C. for 4 h. The reaction was quenched by water and extracted with EA (20 mL×2), and the combined organic layers were concentrated under reduce pressure. The residue was purified by flash chromatography to get the product benzyl 4-[3-[tert-butoxycarbonyl(methyl)amino]propylcarbamoyl]piperidine-1-carboxylate (640 mg, 1.48 mmol, 77.73% yield) as a yellow oil. (ESI⁺) [(M+23)⁺]: 456.3

### Step 2: tert-butyl methyl(3-(piperidine-4-carboxamido)propyl)carbamate

To a solution of benzyl 4-[3-[tert-butoxycarbonyl(methyl)amino]propylcarbamoyl]piperidine-1-carboxylate (500.0 mg, 1.15 mmol, 1 eq) in methanol (10 mL) was added Pd/C (50.0 mg, 1.15 mmol, 1 eq) slowly at 20° C., the mixture was stirred at 20° C. for 16 h under H₂ atmosphere, the mixture was filtered and concentrated to get the product tert-butyl N-methyl-N-[3-(piperidine-4-carbonylamino)propyl]carbamate (360 mg, 1.2 mmol, 88.62% yield) as a yellow oil in crude form. (ESI⁺) [(M+1)⁺]: 300.2

### Step 3: tert-butyl methyl(3-(1-(2-methyl-4-nitrobenzoyl)piperidine-4-carboxamido)propyl)carbamate

To a solution of 2-methyl-4-nitro-benzoic acid (254.11 mg, 1.4 mmol, 1.2 eq) and 2-methyl-4-nitro-benzoic acid (254.11 mg, 1.4 mmol, 1.2 eq) in DMF (5 mL), was added N,N-diisopropylethylamine (0.2 mL, 1.17 mmol, 1 eq) and O-(7-azabenzotriazol-1-yl)-N,N,N′,N′-tetramethyluronium hexafluorophosphate (444.48 mg, 1.17 mmol, 1 eq) at 20° C., the mixture was stirred at 20° C. for 4 h, the mixture was concentrated to get a residue, which was purified by flash chromatography to get the title compound (300 mg, 0.650 mmol, 47.16% yield) as a white solid. (ESI⁺) [(M+23)⁺]: 485.2

### Step 4: tert-butyl (3-(1-(4-amino-2-methylbenzoyl)piperidine-4-carboxamido)propyl)(methyl)carbamate

To a solution of tert-butyl N-methyl-N-[3-[[1-(2-methyl-4-nitro-benzoyl)piperidine-4-carbonyl]amino]propyl]carbamate (50.0 mg, 0.110 mmol, 1 eq) in methanol (10 mL) was added Pd/C (5.0 mg, 0.110 mmol, 1 eq) at 20° C., the mixture was stirred at 20° C. for 16 h under H₂. The mixture was filtered and concentrated and the residue was purified by prep-TLC (DCM:MeOH=10:1) to give the title compound (25 mg, 0.060 mmol, 53.47% yield) as a colorless oil. (ESI⁺) [(M+23)⁺]: 455.2

### Step 5: 2-(4-bromo-2,3-difluorophenoxy)acetonitrile

To a mixture of bromoacetonitrile (2.3 g, 19.14 mmol, 2 eq) and potassium carbonate (2.65 g, 19.14 mmol, 2 eq) in DMF (25 mL) was added bromoacetonitrile (2.3 g, 19.14 mmol, 2 eq) and the mixture was stirred for 12 h at 25° C. The reaction was diluted with water (100 mL) and extracted with ethyl acetate (75 mL×2). The combined organic layers were washed with 50 mL water and 50 mL saturated brine sequentially, dried by MgSO₄ and concentrated to dryness. The crude product was then purified by flash column chromatography eluting 20% ethyl acetate in petroleum ether to give the desired product as light yellow oil. ¹H NMR (400 MHz, CHLOROFORM-d) δ=7.39-7.31 (m, 1H), 6.91-6.81 (m, 1H), 4.87 (d, J=1.3 Hz, 2H) ppm.

### Step 6: 2-(2,3-difluoro-4-(4,4,5,5-tetramethyl-1,3,2-dioxaborolan-2-yl)phenoxy)acetonitrile

The title compound was obtained in analogy to step 4 in the preparation of Intermediate 27 using 2-(4-bromo-2,3-difluorophenoxy)acetonitrile, used in crude form.

### Step 7: 2-(4-(8-chloroimidazo[1,2-a]pyrazin-3-yl)-2,3-difluorophenoxy)acetonitrile

The title compound was obtained in analogy to step 5 in the preparation of Intermediate 27 using 2-(2,3-difluoro-4-(4,4,5,5-tetramethyl-1,3,2-dioxaborolan-2-yl)phenoxy)acetonitrile. (ESI⁺) [(M+1)⁺]:321.0

### Step 8: tert-butyl (3-(1-(4-((3-(4-(cyanomethoxy)-2,3-difluorophenyl)imidazo[1,2-a]pyrazin-8-yl)amino)-2-methylbenzoyl)piperidine-4-carboxamido)propyl)(methyl)carbamate

To a solution of 2-[4-(8-chloroimidazo[1,2-a]pyrazin-3-yl)-2,3-difluoro-phenoxy]acetonitrile (18.53 mg, 0.060 mmol, 1 eq) and tert-butyl N-[3-[[1-(4-amino-2-methyl-benzoyl)piperidine-4-carbonyl]amino]propyl]-N-methyl-carbamate (25.0 mg, 0.060 mmol, 1 eq) in tert-butanol (2 mL) was added potassium carbonate (15.98 mg, 0.120 mmol, 2 eq) and Brettphos Pd G3 (2.62 mg, 0 mmol, 0.050 eq) at 20° C., the mixture was stirred at 80° C. for 4 h, the mixture was filtered and concentrated to give the title compound (20 mg, 0.030 mmol, 44.42% yield) as a yellow oil. (ESI⁺) [(M+1)⁺]: 717.3

### Step 9: 1-(4-((3-(4-(cyanomethoxy)-2,3-difluorophenyl)imidazo[1,2-a]pyrazin-8-yl)amino)-2-methylbenzoyl)-N-(3-(methylamino)propyl)piperidine-4-carboxamide

The title compound was obtained in analogy to step 2, Reference Example 10 using tert-butyl (3-(1-(4-((3-(4-(cyanomethoxy)-2,3-difluorophenyl)imidazo[1,2-a]pyrazin-8-yl)amino)-2-methylbenzoyl)piperidine-4-carboxamido)propyl)(methyl)carbamate. (ESI⁺) [(M+1)]⁺: 617.2

### Reference Example 451

### N-(2-(2-aminoethoxy)ethyl)-4-((3-(4-(cyanomethoxy)-2,3-difluorophenyl)imidazo[1,2-a]pyrazin-8-yl)amino)-2-ethylbenzamide

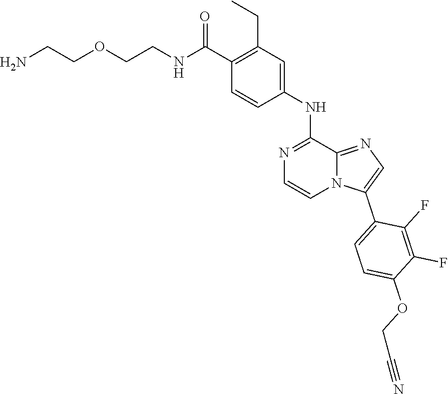 [SMILES: CCc1cc(Nc2nccn3c(-c4ccc(OCC#N)c(F)c4F)cnc23)ccc1C(=O)NCCOCCN]

### Step 1: methyl 4-nitro-2-vinylbenzoate

A solution of methyl 2-bromo-4-nitro-benzoate (15.0 g, 57.68 mmol, 1 eq), vinylboronic acid pinacol ester (13.33 g, 86.53 mmol, 1.5 eq), potassium phosphate (14.33 mL, 173.05 mmol, 3 eq) and 1,1′-bis(di-tert-butylphosphino)ferrocene palladium dichloride (3.76 g, 5.77 mmol, 0.100 eq) in toluene (150 mL) and water (5 mL) was heated to 100° C. for 15 h under nitrogen atmosphere. The reaction mixture was concentrated and the residue was purified by silica gel chromatography eluting with petroleum ether/ethyl acetate=50:1 to afford product methyl 4-nitro-2-vinyl-benzoate (6.2 g, 29.93 mmol, 51.88% yield) as a light yellow solid.

### Step 2: 4-nitro-2-vinylbenzoic Acid

To a solution of methyl 4-nitro-2-vinyl-benzoate (2.0 g, 9.65 mmol, 1 eq) in THF (25 mL) and water (5 mL) was added lithium hydroxide hydrate (0.81 g, 19.31 mmol, 2 eq). The resulting mixture was stirred at 20° C. for 15 h. Then most solvent was removed, the mixture was neutralized with 3 N HCl and extracted with DCM, the obtained organic layer was dried over Na₂SO₄ and concentrated to afford 4-nitro-2-vinyl-benzoic acid (1.68 g, 8.7 mmol, 90.1% yield) as a yellow solid, which was used directly in next step without further purification.

### Step 3: tert-butyl (2-(2-(4-nitro-2-vinylbenzamido)ethoxy)ethyl)carbamate

The title compound was obtained in analogy to step 3, Reference Example 450 using tert-butyl (2-(2-aminoethoxy)ethyl)carbamate and 4-nitro-2-vinylbenzoic acid. (ESI⁺) [(M+23)⁺]: 402.3

### Step 4: tert-butyl (2-(2-(4-amino-2-ethylbenzamido)ethoxy)ethyl)carbamate

The title compound was obtained in analogy to step 4 in Reference Example 450 using tert-butyl (2-(2-(4-nitro-2-vinylbenzamido)ethoxy)ethyl)carbamate. (ESI⁺) [(M+23)]⁺: 374.1

### Step 5: tert-butyl (2-(2-(4-((3-(4-(cyanomethoxy)-2,3-difluorophenyl)imidazo[1,2-a]pyrazin-8-yl)amino)-2-ethylbenzamido)ethoxy)ethyl)carbamate

The title compound was obtained in analogy to step 8 in Reference Example 450 using 2-(4-(8-chloroimidazo[1,2-a]pyrazin-3-yl)-2,3-difluorophenoxy)acetonitrile and tert-butyl (2-(2-(4-amino-2-ethylbenzamido)ethoxy)ethyl)carbamate. (ESI⁺) [(M+1)⁺]:636.1

### Step 6: N-(2-(2-aminoethoxy)ethyl)-4-((3-(4-(cyanomethoxy)-2,3-difluorophenyl)imidazo[1,2-a]pyrazin-8-yl)amino)-2-ethylbenzamide

The title compound was obtained in analogy to step 2, Reference Example 10 using tert-butyl (2-(2-(4-((3-(4-(cyanomethoxy)-2,3-difluorophenyl)imidazo[1,2-a]pyrazin-8-yl)amino)-2-ethylbenzamido)ethoxy)ethyl)carbamate. (ESI⁺) [(M+1)⁺]: 536.4

### Example 44

### 1-(2-bromo-4-((3-(3-fluoro-4-methoxyphenyl)imidazo[1,2-a]pyrazin-8-yl)amino)benzoyl)-N-(2-(methylamino)ethyl)piperidine-4-carboxamide; Formic Acid

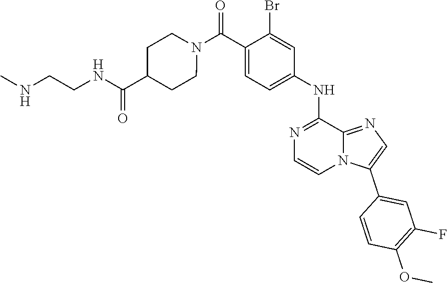 [SMILES: CNCCNC(=O)C1CCN(C(=O)c2ccc(Nc3nccn4c(-c5ccc(OC)c(F)c5)cnc34)cc2Br)CC1]

### Step 1: benzyl 4-((2-((tert-butoxycarbonyl)(methyl)amino)ethyl)carbamoyl)piperidine-1-carboxylate; Formic Acid

To a solution of 1-[(benzyloxy)carbonyl]piperidine-4-carboxylic acid (1.0 g, 3.8 mmol, 1 eq) in DCM (30 mL) was added N-(2-aminoethyl)-N-methyl carbamic acid tert-butyl ester (727.96 mg, 4.18 mmol, 1.1 eq), triethylamine (1.59 mL, 11.39 mmol, 3 eq) and 1-propanephosphonic anhydride (3625.43 mg, 5.7 mmol, 1.5 eq). The mixture was stirred at 25° C. for 6 h. The reaction mixture was washed with aqueous hydrochloric acid, dried over magnesium sulfate, filtered and the filtrate concentrated in vacuo. The residue was purified by prep-HPLC (FA as modifier) to give benzyl 4-[2-[tert-butoxycarbonyl(methyl)amino]ethylcarbamoyl]piperidine-1-carboxylate (1.3 g, 3.1 mmol, 81.59% yield) as colorless oil. MS (ESI+) [M-Boc+H]⁺: 320

### Step 2: tert-butyl methyl(2-(piperidine-4-carboxamido)ethyl)carbamate

To a solution of benzyl 4-[2-[tert-butoxycarbonyl(methyl)amino]ethylcarbamoyl]piperidine-1-carboxylate (1200.0 mg, 2.86 mmol, 1 eq) in ethyl acetate (30 mL) was added Pd/C (302.92 mg, 0.290 mmol, 0.100 eq). The mixture was stirred at 25° C. for 14 h under H₂ balloon. The mixture was filtered through a Celite pad, and the filtrate was concentrated to give tert-butyl N-methyl-N-[2-(piperidine-4-carbonylamino)ethyl]carbamate (750 mg, 2.63 mmol, 91.88% yield) as black oil. MS (ESI⁺) [M+H]⁺: 286

### Step 3: methyl 2-bromo-4-((3-iodoimidazo[1,2-a]pyrazin-8-yl)amino)benzoate

To a solution of 8-chloro-3-iodo-imidazo[1,2-a]pyrazine (2.0 g, 7.16 mmol, 1 eq) in MeCN (18 mL)/acetic acid (2 mL) was added methyl 4-amino-2-bromo-benzoate (1646.4 mg, 7.16 mmol, 1 eq). The mixture was stirred at 90° C. for 14 h. The mixture was filtered and the solid was washed by acetonitrile to give methyl 2-bromo-4-[(3-iodoimidazo[1,2-a]pyrazin-8-yl)amino]benzoate (2.8 g, 5.92 mmol, 73% yield) as white solid. MS (ESI⁺) [M+H]⁺: 474.7

### Step 4: methyl 2-bromo-4-((3-(3-fluoro-4-methoxyphenyl)imidazo[1,2-a]pyrazin-8-yl)amino)benzoate

The title compound was obtained in analogy to step 5 in the preparation of Intermediate 27 using methyl 2-bromo-4-((3-iodoimidazo[1,2-a]pyrazin-8-yl)amino)benzoate and 2-(3-fluoro-4-methoxyphenyl)-4,4,5,5-tetramethyl-1,3,2-dioxaborolane. MS (ESI⁺) [M+H]⁺: 472.8

### Step 5: 2-bromo-4-((3-(3-fluoro-4-methoxyphenyl)imidazo[1,2-a]pyrazin-8-yl)amino)benzoic Acid

A solution of methyl 2-bromo-4-[[3-(3-fluoro-4-methoxy-phenyl)imidazo[1,2-a]pyrazin-8-yl]amino]benzoate (200.0 mg, 0.420 mmol, 1 eq) in methanol (10 mL) was stirred at 80° C. for 10 min. Then 4M aq. sodium hydroxide (5.0 mL, 20 mmol, 47.13 eq) was added. The mixture was stirred at 80° C. for 4 h. The reaction mixture was concentrated in vacuo, diluted with water, and acidified with 2 N HCl. The solid was collected and thoroughly dried to give 2-bromo-4-[[3-(3-fluoro-4-methoxy-phenyl)imidazo[1,2-a]pyrazin-8-yl]amino]benzoic acid (180 mg, 0.390 mmol, 64.93% yield) as off white solid. (ESI⁺) [M+H]⁺: 458.9

### Step 6: 1-(2-bromo-4-((3-(3-fluoro-4-methoxyphenyl)imidazo[1,2-a]pyrazin-8-yl)amino)benzoyl)-N-(2-(methylamino)ethyl)piperidine-4-carboxamide; Formic Acid

To a mixture of O-(7-azabenzotriazol-1-yl)-N,N,N′,N′-tetramethyluronium hexafluorophosphate (99.79 mg, 0.260 mmol, 1.2 eq) and triethylamine (0.06 mL, 0.440 mmol, 2 eq) in DMF (4 mL) was added 2-bromo-4-[[3-(3-fluoro-4-methoxy-phenyl)imidazo[1,2-a]pyrazin-8-yl]amino]benzoic acid (100.0 mg, 0.220 mmol, 1 eq) and tert-butyl N-methyl-N-[2-(piperidine-4-carbonylamino)ethyl]carbamate (93.62 mg, 0.330 mmol, 1.5 eq) slowly at 25° C. Then the mixture was stirred at 25° C. for 12 h. Then to the mixture was added HCl indioxane (3 mL). The mixture was stirred at 25° C. for 4 h. The mixture was filtered and the filtrate was purified by prep-HPLC (FA as additive) to give 1-[2-bromo-4-[[3-(3-fluoro-4-methoxy-phenyl)imidazo[1,2-a]pyrazin-8-yl]amino]benzoyl]-N-[2-(methylamino)ethyl]piperidine-4-carboxamide (40 mg, 0.060 mmol, 26.73% yield) as light yellow solid. MS (ESI⁺) [M+H]⁺: 626

### Example 45

### (2S)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic Acid; Formic Acid

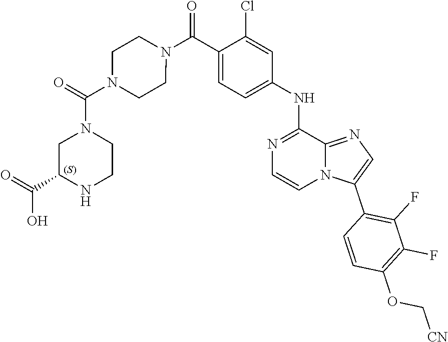

### Step 1: (S)-1-tert-butyl 2-methyl 4-(piperidine-4-carbonyl)piperazine-1,2-dicarboxylate

To a mixture of 1-benzylpiperidine-4-carboxylic acid (300 mg, 1.37 mmol), (S)-1-tert-butyl 2-methyl piperazine-1,2-dicarboxylate (501 mg, 2.05 mmol) and triethylamine (0.57 mL, 4.1 mmol) in DMF (10 mL) was added dropwise 1-propanephosphonic anhydride (1295 mg, 2.05 mmol) at 25° C. under N₂. The mixture was stirred at 25° C. for 2 hours. The reaction mixture was poured into water (50 mL), it was extracted by ethyl acetate (50 mL×3), the combined organic layers were washed by brine (50 mL), dried by Na₂SO₄, filtered and concentrated to give the crude intermediate. A mixture of above residue (400 mg, 0.900 mmol) and palladium hydroxide on carbon (40 mg, 0.900 mmol) in methanol (10 mL) was stirred at 25° C. under a hydrogen balloon for 16 hours. The reaction mixture was filtered and concentrated to afford (S)-1-tert-butyl 2-methyl 4-(piperidine-4-carbonyl)piperazine-1,2-dicarboxylate (220 mg, 0.620 mmol, 69% yield) as a colorless oil. MS (ESI⁺) [M+H]⁺: 356.1

### Step 2: 2-chloro-4-((3-iodoimidazo[1,2-a]pyrazin-8-yl)amino)benzoic acid

The title compound was obtained in analogy to step 3 of Example 44 using 8-chloro-3-iodoimidazo[1,2-a]pyrazine and 4-amino-2-chlorobenzoic acid. MS (ESI⁺) [M+H]⁺: 415.0

### Step 3: (S)-1-tert-butyl 2-methyl 4-(1-(2-chloro-4-((3-iodoimidazo[1,2-a]pyrazin-8-yl)amino)benzoyl)piperidine-4-carbonyl)piperazine-1,2-dicarboxylate

To a mixture of 2-chloro-4-((3-iodoimidazo[1,2-a]pyrazin-8-yl)amino)benzoic acid (2.0 g, 4.82 mmol), N-hydroxysuccinimide (0.72 g, 6.27 mmol) in DMF (10 mL) and THF (30 mL) was added 1-ethyl-(3-(3-dimethylamino)propyl)-carbodiimide hydrochloride (1.4 mL, 5.31 mmol) at 25° C. under N₂. The mixture was stirred at 25° C. for 16 hours. LC-MS indicated the reaction was completed. The reaction mixture was concentrated and the residue was poured into water (50 mL). The mixture was filtered and the filtrate was concentrated to afford a residue (1.1 g, 2.15 mmol) as a white solid. A mixture of the above residue (303 mg, 0.590 mmol), (S)-1-tert-butyl 2-methyl 4-(piperidine-4-carbonyl)piperazine-1,2-dicarboxylate (220 mg, 0.590 mmol) and triethylamine (0.12 mL, 0.890 mmol) in THF (5 mL) was stirred at 25° C. for 2 h. The reaction mixture was concentrated to afford (S)-1-tert-butyl 2-methyl 4-(1-(2-chloro-4-((3-iodoimidazo[1,2-a]pyrazin-8-yl)amino)benzoyl)piperidine-4-carbonyl)piperazine-1,2-dicarboxylate (350 mg, 0.470 mmol) as a white solid. MS (ESI⁺) [M+H]⁺: 752.1

### Step 4: (S)-1-tert-butyl 2-methyl 4-(1-(2-chloro-4-((3-(4-(cyanomethoxy)-2,3-difluorophenyl)imidazo[1,2-a]pyrazin-8-yl)amino)benzoyl)piperidine-4-carbonyl)piperazine-1,2-dicarboxylate

The title compound was obtained in analogy to step 5 in the preparation of Intermediate 27 using (S)-1-tert-butyl 2-methyl 4-(1-(2-chloro-4-((3-iodoimidazo[1,2-a]pyrazin-8-yl)amino)benzoyl)piperidine-4-carbonyl)piperazine-1,2-dicarboxylate and 2-(2,3-difluoro-4-(4,4,5,5-tetramethyl-1,3,2-dioxaborolan-2-yl)phenoxy)acetonitrile. (ESI⁺) [(M+H)⁺]: 793.0

### Step 5: (2S)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic Acid; Formic Acid

A mixture of (S)-1-tert-butyl 2-methyl 4-(1-(2-chloro-4-((3-(4-(cyanomethoxy)-2,3-difluorophenyl)imidazo[1,2-a]pyrazin-8-yl)amino)benzoyl)piperidine-4-carbonyl)piperazine-1,2-dicarboxylate (100 mg, 0.130 mmol) and lithium chloride (107 mg, 2.52 mmol) in triethylamine (0.1 mL, 0.720 mmol) and DMF (0.5 mL) was stirred at 100° C. under N₂ for 16 hours. To the mixture was added dropwise trifluoroacetic acid (0.2 mL, 2.6 mmol) at 25° C. The mixture was stirred at 25° C. under N₂ for 16 hours. The reaction mixture was purified directly via prep-HPLC and then lyophilized to afford the title compound (7.5 mg, 0.010 mmol) as a white solid. (ESI⁺) [M+H]⁺: 679.0

### Example 46

### (R)-4-(1-(2-chloro-4-((3-(4-(cyanomethoxy)-2,3-difluorophenyl)imidazo[1,2-a]pyrazin-8-yl)amino)benzoyl)piperidine-4-carbonyl)piperazine-2-carboxylic Acid; Formic Acid

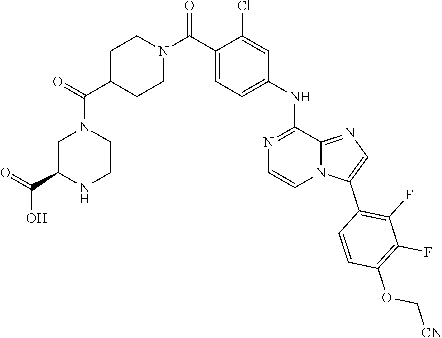 [SMILES: N#CCOc1ccc(-c2cnc3c(Nc4ccc(C(=O)N5CCC(C(=O)N6CCN[C@@H](C(=O)O)C6)CC5)c(Cl)c4)nccn23)c(F)c1F]

The title compound was obtained in analogy to Example 45 via a 5-step sequence using (R)-1-tert-butyl 2-methyl piperazine-1,2-dicarboxylate instead of (S)-1-tert-butyl 2-methyl piperazine-1,2-dicarboxylate.

(ESI⁺) [M+H]⁺. 679.0

### Intermediate 137

### 2-[4-[8-[3-chloro-4-[4-(2-chloroethyl)piperazine-1-carbonyl]anilino]imidazo[1,2-a]pyrazin-3-yl]-2,3-difluoro-phenoxy]acetonitrile

### Step 1: 1-(2-chloroethyl)piperazine bis(2,2,2-trifluoroacetate)

To a solution of tert-butyl 4-(2-chloroethyl)piperazine-1-carboxylate (1 g, 4.02 mmol) in DCM (10 mL)/TFA (10 mL), the reaction was stirred for two hours at room temperature. The reaction mixture was concentrated in vacuum to give 1-(2-chloroethyl)piperazine bis(2,2,2-trifluoroacetate) (1.48 g).

### Step 2: 2-[4-[8-[3-chloro-4-[4-(2-chloroethyl)piperazine-1-carbonyl]anilino]imidazo[1,2-a]pyrazin-3-yl]-2,3-difluoro-phenoxy]acetonitrile

To a solution of Intermediate 29 (200 mg, 439 μmol) in DMF (5 mL) was added 1-(2-chloroethyl)piperazine bis(2,2,2-trifluoroacetate) (165 mg, 439 μmol), triethylamine (178 mg, 245 μL, 1.76 mmol) and 2,4,6-tripropyl-1,3,5,2,4,6-trioxatriphosphinane 2,4,6-trioxide (411 μL, 658 μmol), the reaction was stirred for two hours at room temperature. The mixture was washed with brine and extracted in DCM. The organic layer was concentrated in vacuo to give crude 2-(4-(8-((3-chloro-4-(4-(2-chloroethyl)piperazine-1-carbonyl)phenyl)amino)imidazo[1,2-a]pyrazin-3-yl)-2,3-difluorophenoxy)acetonitrile (270 mg, 428 μmol, 97.6% yield). MS (ESI) [M+H]⁺: 586.1

### Example 47

### 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylic Acid Trifluoroacetate

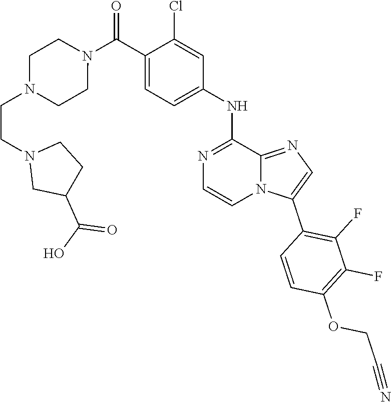 [SMILES: N#CCOc1ccc(-c2cnc3c(Nc4ccc(C(=O)N5CCN(CCN6CCC(C(=O)O)C6)CC5)c(Cl)c4)nccn23)c(F)c1F]

### Step 1: tert-butyl 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylate

To a solution of Intermediate 137 (80 mg, 136 μmol) in DMSO (3 mL) was added sodium carbonate (28.9 mg, 273 μmol) and tert-butyl pyrrolidine-3-carboxylate (46.7 mg, 273 μmol), the reaction was stirred for 15 hours at 60° C. The reaction mixture was cooled to room temperature and filtered. The filtrate was poured into water and the mixture was filtered. The filter cake was dried in vacuum to give tert-butyl 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylate (100 mg, 128 μmol, 93.5% yield).

### Step 2: 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylic Acid Trifluoroacetate

To a solution of tert-butyl 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylate (100 mg, 128 μmol) in THF (5 mL) was added 6N HCl (2 mL), the reaction mixture was stirred for two hours at room temperature. The mixture was neutralized with ammonium hydroxide. The mixture was extracted in ethyl acetate and the organic layer was concentrated in vacuum. The residue was purified by preparative HPLC to give 1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylic acid (16 mg). MS (ESI) [M+H]+: 665.1

The following examples were prepared in analogy to Example 47, the hydrolysis of tert butyl ester step 2 was only applied for intermediates containing a tert butyl ester group.

| | | | ESI | |
|---|---|---|---|---|
| Ex. | Name | Structure | [M + H]⁺ | Starting Material |
| 48 | 2-[4-[8-[4-[4-[2- (azetidin-1- yl)ethyl]piperazine- 1-carbonyl]-3- chloro- anilino]imidazo[1,2- a]pyrazin-3-yl]-2,3- difluoro- phenoxy]acetonitrile trifluoroacetate | 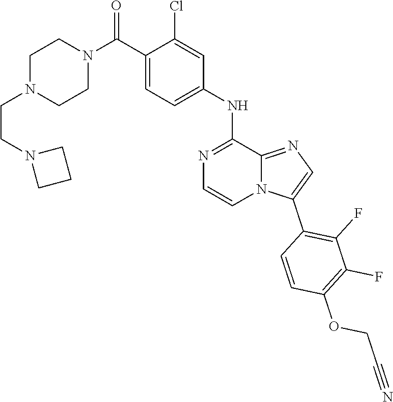 [SMILES: N#CCOc1ccc(-c2cnc3c(Nc4ccc(C(=O)N5CCN(CCN6CCC6)CC5)c(Cl)c4)nccn23)c(F)c1F] | 607.1 | Intermediate 137 and azetidine hydrochloride |

### Reference Example 452

### N-[2-(2-aminoethoxy)ethyl]-4-[[3-(2,6-difluoro-4-methoxyphenyl)imidazo[1,2-a]pyrazin-8-yl]amino]-2-ethylbenzamide Hydrochloride

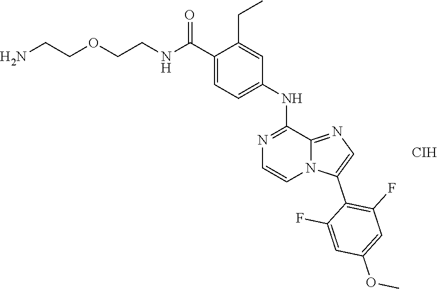

### Step 1

### tert-butyl N-[2-[2-[[2-ethyl-4-[(3-iodoimidazo[1,2-a]pyrazin-8-yl)amino]benzoyl]amino]ethoxy]ethyl]carbamate (Intermediate 138)

A mixture Intermediate 63 (1.421 g, 3.2 mmol), triethylamine (1.62 g, 2.23 mL, 16 mmol), TBTU (1.22 g, 3.68 mmol) and tert-butyl (2-(2-aminoethoxy)ethyl)carbamate (816 mg, 3.99 mmol) in DMF (20 mL) was stirred at room temperature overnight. The reaction mixture was poured into 150 mL water and extracted with ethyl acetate (2×100 mL). The crude material was adsorbed on Isolute and purified by flash chromatography (silica gel, 80 g, 0% to 100% ethyl acetate in heptane) to yield tert-butyl (2-(2-(2-ethyl-4-((3-iodoimidazo[1,2-a]pyrazin-8-yl)amino)benzamido)ethoxy)ethyl)carbamate (1.561 g, 2.63 mmol, 82.2%). MS (ESI, m/z): 595.4 [M+H]⁺.

### Step 2

### tert-butyl N-[2-[2-[[4-[[3-(2,6-difluoro-4-methoxy-phenyl)imidazo[1,2-a]pyrazin-8-yl]amino]-2-ethyl-benzoyl]amino]ethoxy]ethyl]carbamate

A mixture of tert-butyl N-[2-[2-[[2-ethyl-4-[(3-iodoimidazo[1,2-a]pyrazin-8-yl)amino]benzoyl]amino]ethoxy]ethyl]carbamate (Intermediate 138) (89.2 mg, 150 μmol), (2,6-difluoro-4-methoxyphenyl)boronic acid (19.7 mg, 225 μmol), 1,1′-bis (diphenylphosphino)ferrocene-palladium(II)dichloride dichloromethane complex (12.2 mg, 15 μmol) and Na₂CO₃ (31.8 mg, 300 μmol) in dioxane (1 mL)/water (0.1 mL) was stirred at 110° C. overnight. The mixtures were concentrated in vacuo, pre-purified by passing through a 4 g silica column eluting with 30 mL of a ethyl acetate/MeOH 9/1 solution and concentrated. Purification with preparative HPLC on reversed phase (Gemini 5 um C18 75×30) eluting with a gradient formed from water (+0.1% NEt₃)/acetonitrile yielded after evaporation of the product containing fractions tert-butyl N-[2-[2-[[4-[[3-(2,6-difluoro-4-methoxy-phenyl)imidazo[1,2-a]pyrazin-8-yl]amino]-2-ethyl-benzoyl]amino]ethoxy]ethyl]carbamate (7.8 mg, 12.8 μmol, 8.5%). MS (ESI, m/z): 611.4 [M+H]+.

### Step 3

### Reference Example 452

### N-[2-(2-aminoethoxy)ethyl]-4-[[3-(2,6-difluoro-4-methoxyphenyl)imidazo[1,2-a]pyrazin-8-yl]amino]-2-ethylbenzamide;hydrochloride

A mixture of tert-butyl N-[2-[2-[[4-[[3-(2,6-difluoro-4-methoxy-phenyl)imidazo[1,2-a]pyrazin-8-yl]amino]-2-ethyl-benzoyl]amino]ethoxy]ethyl]carbamate and excess 4N HCl (dioxane) in DCM (2 mL) was stirred at room temperature for 2 h and evaporated to dryness. The residue was triturated with 2 mL of Et₂O and the product was filtered off to yield after drying N-(2-(2-aminoethoxy)ethyl)-4-((3-(2,6-difluoro-4-methoxyphenyl)imidazo[1,2-a]pyrazin-8-yl)amino)-2-ethylbenzamide hydrochloride (4.8 mg, 9.4 μmol, 73.5%). MS (ESI, m/z): 509.4 [M+H]+.

### Reference Example 453

### N-[2-(2-aminoethoxy)ethyl]-2-ethyl-4-[[3-[4-methoxy-2-(methylsulfamoyl)phenyl]imidazo[1,2-a]pyrazin-8-yl]amino]benzamide;formic Acid

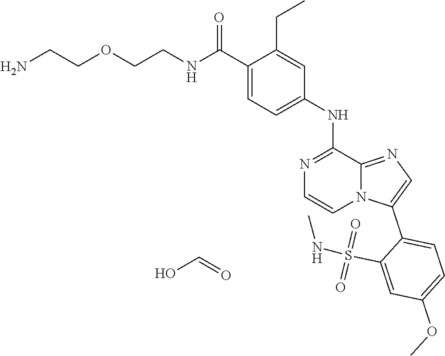 [SMILES: CCc1cc(Nc2nccn3c(-c4ccc(OC)cc4S(=O)(=O)NC)cnc23)ccc1C(=O)NCCOCCN ; O=CO]

### Step 1

### tert-butyl N-[2-[2-[[2-ethyl-4-[[3-[4-methoxy-2-(methylsulfamoyl)phenyl]imidazo[1,2-a]pyrazin-8-yl]amino]benzoyl]amino]ethoxy]ethyl]carbamate

In analogy to the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl N-[2-[2-[[2-ethyl-4-[(3-iodoimidazo[1,2-a]pyrazin-8-yl)amino]benzoyl]amino]ethoxy]ethyl]carbamate (Intermediate 138) and 5-methoxy-N-methyl-2-(4,4,5,5-tetramethyl-1,3,2-dioxaborolan-2-yl)benzenesulfonamide. MS (ESI, m/z): 668.4 [M+H]+.

### Step 2

### N-[2-(2-aminoethoxy)ethyl]-2-ethyl-4-[[3-[4-methoxy-2-(methylsulfamoyl)phenyl]imidazo[1,2-a]pyrazin-8-yl]amino]benzamide Formate

In analogy the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl N-[2-[2-[[2-ethyl-4-[[3-[4-methoxy-2-(methylsulfamoyl)phenyl]imidazo[1,2-a]pyrazin-8-yl]amino]benzoyl]amino]ethoxy]ethyl]carbamate through acidic cleavage of the protecting group followed by reversed phase column chromatography eluting with a gradient formed from water (+0.1% formic acid)/acetonitrile. Evaporation of the product containing fractions yielded the title compound. MS (ESI, m/z): 568.3 [M+H]+.

### Reference Example 454

### N-(2-(2-aminoethoxy)ethyl)-2-ethyl-4-((3-(4-methoxy-2-(trifluoromethyl)phenyl)imidazo[1,2-a]pyrazin-8-yl)amino)benzamide Hydrochloride

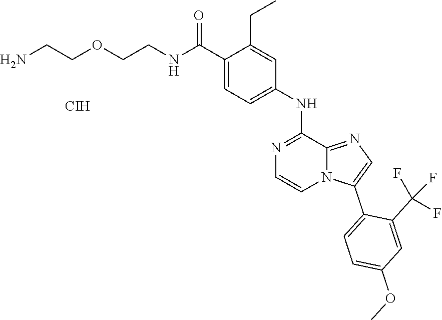 [SMILES: CCc1cc(Nc2nccn3c(-c4ccc(OC)cc4C(F)(F)F)cnc23)ccc1C(=O)NCCOCCN ; "CIH"]

### Step 1

### tert-butyl (2-(2-(2-ethyl-4-((3-(4-methoxy-2-(trifluoromethyl)phenyl)imidazo[1,2-a]pyrazin-8-yl)amino)benzamido)ethoxy)ethyl)carbamate

In analogy to the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl N-[2-[2-[[2-ethyl-4-[(3-iodoimidazo[1,2-a]pyrazin-8-yl)amino]benzoyl]amino]ethoxy]ethyl]carbamate (Intermediate 138) and 2-(4-methoxy-2-(trifluoromethyl)phenyl)-4,4,5,5-tetramethyl-1,3,2-dioxaborolane. MS (ESI, m/z): 643.3 [M+H]+.

### Step 2

### N-(2-(2-aminoethoxy)ethyl)-2-ethyl-4-((3-(4-methoxy-2-(trifluoromethyl)phenyl)imidazo[1,2-a]pyrazin-8-yl)amino)benzamide Hydrochloride

In analogy the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl (2-(2-(2-ethyl-4-((3-(4-methoxy-2-(trifluoromethyl)phenyl)imidazo[1,2-a]pyrazin-8-yl)amino)benzamido)ethoxy)ethyl)carbamate through acidic cleavage of the protecting group. MS (ESI, m/z): 543.3 [M+H]+.

### Reference Example 455

### 4-((3-(2-amino-4-methoxyphenyl)imidazo[1,2-a]pyrazin-8-yl)amino)-N-(2-(2-aminoethoxy)ethyl)-2-ethylbenzamide Hydrochloride

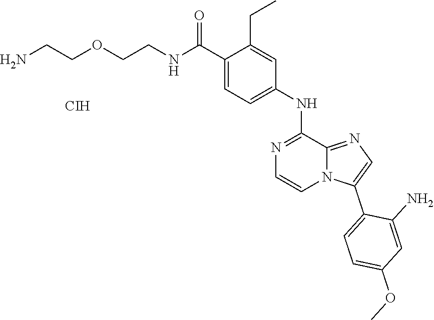 [SMILES: CCc1cc(Nc2nccn3c(-c4ccc(OC)cc4N)cnc23)ccc1C(=O)NCCOCCN ; "CIH"]

### Step 1

### tert-butyl (2-(2-(4-((3-(2-((tert-butoxycarbonyl)amino)-4-methoxyphenyl)imidazo[1,2-a]pyrazin-8-yl)amino)-2-ethylbenzamido)ethoxy)ethyl)carbamate

In analogy to the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl N-[2-[2-[[2-ethyl-4-[(3-iodoimidazo[1,2-a]pyrazin-8-yl)amino]benzoyl]amino]ethoxy]ethyl]carbamate (Intermediate 138) and tert-butyl (5-methoxy-2-(4,4,5,5-tetramethyl-1,3,2-dioxaborolan-2-yl)phenyl)carbamate. MS (ESI, m/z): 690.5 [M+H]+.

### Step 2

### 4-((3-(2-amino-4-methoxyphenyl)imidazo[1,2-a]pyrazin-8-yl)amino)-N-(2-(2-aminoethoxy)ethyl)-2-ethylbenzamide Hydrochloride

In analogy the procedure described for the synthesis of Reference Example 452 N-[2-(2-aminoethoxy)ethyl]-4-[[3-(2,6-difluoro-4-methoxyphenyl)imidazo[1,2-a]pyrazin-8-yl]amino]-2-ethylbenzamide hydrochloride the title compound was prepared from tert-butyl (2-(2-(4-((3-(2-((tert-butoxycarbonyl)amino)-4-methoxyphenyl)imidazo[1,2-a]pyrazin-8-yl)amino)-2-ethylbenzamido)ethoxy)ethyl)carbamate through acidic cleavage of the protecting group. MS (ESI, m/z): 490.4 [M+H]+.

### Intermediate 139

### 3-(2,3-difluoro-4-methoxyphenyl)imidazo[1,2-a]pyrazin-8-amine

In a sealed pressure tube a suspension of 8-chloro-3-(2,3-difluoro-4-methoxyphenyl)imidazo[1,2-a]pyrazine (Intermediate 21, 0.062 g, 210 μmol, Eq: 1) in isopropanol (839 μl) and 25% aq ammonia (1.31 g, 1.46 mL, 19.3 mmol, Eq: 92) was heated to 115° C. for 19 h. The reaction mixture was diluted with water, the suspension filtered and washed with water. The solid was collected and dried in vacuo. The compound 3-(2,3-difluoro-4-methoxyphenyl)imidazo[1,2-a]pyrazin-8-amine (0.046 g, 167 μmol, 79.4% yield) was obtained as light brown solid. MS ESI (m/z): 277.2 [M+H]⁺

### Intermediate 140

### Tert-butyl (2S,4R)-4-hydroxy-2-(piperazine-1-carbonyl)pyrrolidine-1-carboxylate

### Step 1: benzyl 4-[(2S,4R)-1-tert-butoxycarbonyl-4-hydroxy-pyrrolidine-2-carbonyl]piperazine-1-carboxylate

To a clear solution of (2S,4R)-1-(tert-butoxycarbonyl)-4-hydroxypyrrolidine-2-carboxylic acid (1.5 g, 6.29 mmol, Eq: 1) and DIPEA (1.63 g, 2.2 mL, 12.6 mmol, Eq: 2) in dry DMF (15.7 mL) was added HATU (2.39 g, 6.29 mmol, Eq: 1) and the mixture stirred 10 minutes at room temperature. Then a solution of benzyl piperazine-1-carboxylate (1.41 g, 6.29 mmol, Eq: 1) in dry DMF (15.7 mL) was added and stirring at room temperature was continued for 2 h. Then the reaction mixture was concentrated in vacuo. The crude material was purified by silica gel chromatography using heptane/(ethyl acetate/EtOH/NH₄OH 75:25:2) as eluent. The compound benzyl 4-((2S,4R)-1-(tert-butoxycarbonyl)-4-hydroxypyrrolidine-2-carbonyl)piperazine-1-carboxylate (3.177 g, 5.79 mmol, 92% yield) was obtained as yellow oil with an purity of 79% (contains 21% DMF acc to NMR) and was used without further purification. MS ESI (m/z): 478.2184 [M+HCOO⁻]⁻

### Step 2: tert-butyl (2S,4R)-4-hydroxy-2-(piperazine-1-carbonyl)pyrrolidine-1-carboxylate

A flask containing a solution of benzyl 4-((2S,4R)-1-(tert-butoxycarbonyl)-4-hydroxypyrrolidine-2-carbonyl)piperazine-1-carboxylate (3.17 g, 5.78 mmol, Eq: 1) in methanol (38.5 mL) was evacuated 3× (frothing) and flushed with argon. Then 10% palladium on carbon (123 mg, 116 μmol, Eq: 0.02) was added and degassing repeated. Then the apparatus was again 4× evacuated (frothing) and flushed with hydrogen. The reaction was stirred 5 hours at room temperature under hydrogen. Then the reaction was filtered through a glass fibre filter, washed with MeOH and the obtained solution concentrated in vacuo. The obtained material was triturated with heptane/diisopropyl ether, filtered washed and dried in vacuo. The compound tert-butyl (2S,4R)-4-hydroxy-2-(piperazine-1-carbonyl)pyrrolidine-1-carboxylate (1.660 g, 5.38 mmol, 93.1% yield) was obtained as light yellow solid. MS ESI (m/z): 300.2 [M+H]⁺

### Intermediate 141

### Tert-butyl (E)-4-(N,N′-bis(tert-butoxycarbonyl)-1H-pyrazole-1-carboximidamido)butanoate

Biorg and Med Chem Lett 2014, vol 24 #23 p 5525-5529

A solution of tert-butyl (E)-(((tert-butoxycarbonyl)imino)(1H-pyrazol-1-yl)methyl)carbamate (0.155 g, 484 μmol, Eq: 1), triphenylphosphine (201 mg, 727 μmol, Eq: 1.5) and tert-butyl 4-hydroxybutanoate (101 mg, 630 μmol, Eq: 1.3) in dry THF (1.86 mL) was cooled to 0° C. Then DIAD (156 mg, 150 μL, 727 μmol, Eq: 1.5) was added dropwise. Then the cooling bath was removed and the reaction heated to reflux for 16 hours. Then the reaction was quenched with water and diluted with dichloromethane. The mixture was extracted 2× with dichloromethane and the organic layers were washed 1× with water. The combined organic layers were dried with sodium sulfate, filtered and concentrated in vacuo. The crude material was purified by silica gel chromatography using heptane/ethyl acetate as eluent. The compound tert-butyl (E)-4-(N,N′-bis(tert-butoxycarbonyl)-1H-pyrazole-1-carboximidamido)butanoate (52 mg, 107 μmol, 22.1% yield) was obtained as colorless oil with a purity of 93% (UV, 220 nm). MS ESI (m/z): 453.4 [M+H]⁺, ¹H NMR (300 MHz, chloroform-d) 6=7.94 (br s, 1H), 7.68 (dd, J=0.6, 1.6 Hz, 1H), 6.41 (dd, J=1.6, 2.8 Hz, 1H), 3.72 (br t, J=7.4 Hz, 2H), 2.32 (t, J=7.5 Hz, 2H), 2.01 (quin, J=7.4 Hz, 2H), 1.50 (s, 9H), 1.43 (s, 9H), 1.27 (s, 9H)

### Reference Example 456

### N-(2-(2-Aminoethoxy)ethyl)-4-((3-(2,3-difluoro-4-methoxyphenyl)imidazo[1,2-a]pyrazin-8-yl)amino)-2-fluoro-6-methylbenzamide Formate

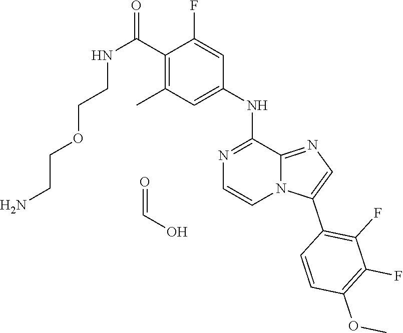 [SMILES: COc1ccc(-c2cnc3c(Nc4cc(C)c(C(=O)NCCOCCN)c(F)c4)nccn23)c(F)c1F ; O=CO]

### Step 1: tert-Butyl 4-bromo-2-fluoro-6-methylbenzoate

In a pressure tube to a white suspension of 4-bromo-2-fluoro-6-methylbenzoic acid (700 mg, 3 mmol, Eq: 1) in dry toluene (1.88 mL) was added N,N-dimethylformamide di-tert-butyl acetal (4.41 g, 5.19 mL, 19.5 mmol, Eq: 6.5). The tube was sealed and the mixture heated to 80° C. for 3 hours. The reaction mixture was diluted with water, ethyl acetate and sat. aqueous NaHCO₃ solution. The mixture was extracted 2× with ethyl acetate and the organic layers washed 1× with sat. aqueous NaHCO₃ solution and 2× with brine. The combined organic layers were dried with sodium sulfate, filtered and concentrated in vacuo. The crude material (drypack on silica gel) was purified by silica gel chromatography using heptane/ethyl acetate as eluent. The compound tert-butyl 4-bromo-2-fluoro-6-methylbenzoate (794.9 mg, 2.69 mmol, 89.7% yield) was obtained as colorless oil and was used without further purification. MS EI (m/z): 290.0 [M]⁺

### Step 2: tert-Butyl 4-((3-(2,3-difluoro-4-methoxyphenyl)imidazo[1,2-a]pyrazin-8-yl)amino)-2-fluoro-6-methylbenzoate

A brown suspension of 3-(2,3-difluoro-4-methoxyphenyl)imidazo[1,2-a]pyrazin-8-amine (Intermediate 139, 300 mg, 1.09 mmol, Eq: 1), tert-butyl 4-bromo-2-fluoro-6-methylbenzoate (628 mg, 2.17 mmol, Eq: 2) and sodium tert-butoxide (157 mg, 1.63 mmol, Eq: 1.5) in THF (10.9 mL) in a pressure tube was sparged with argon for 5 minutes while sonicating the vessel in an ultra-sonic bath. Then 1,1′-bis(diphenylphosphino)ferrocene (72.2 mg, 130 μmol, Eq: 0.12) and tris(dibenzylideneacetone)dipalladium (0) (39.8 mg, 43.4 μmol, Eq: 0.04) were added and degassing continued for 1 minute. The tube was sealed and heated to 130° C. for 3 hours. Then the mixture was concentrated in vacuo. The crude material (drypack on silica gel) was purified by silica gel chromatography using heptane/ethyl acetate as eluent. The compound tert-butyl 4-((3-(2,3-difluoro-4-methoxyphenyl)imidazo[1,2-a]pyrazin-8-yl)amino)-2-fluoro-6-methylbenzoate (367 mg, 758 μmol, 69.8% yield) was obtained as yellow solid and was used without further purification. MS ESI (m/z): 485.2 [M+H]⁺

### Step 3: 4-((3-(2,3-Difluoro-4-methoxyphenyl)imidazo[1,2-a]pyrazin-8-yl)amino)-2-fluoro-6-methylbenzoic Acid Hydrochloride

To a solution of tert-butyl 4-((3-(2,3-difluoro-4-methoxyphenyl)imidazo[1,2-a]pyrazin-8-yl)amino)-2-fluoro-6-methylbenzoate (367 mg, 758 μmol, Eq: 1) in dioxane (1.52 mL) was added 4 M HCl in dioxane (11.4 mL, 45.5 mmol, Eq: 60) and the reaction was heated to 70° C. for 3 hours. Then again 4 M HCl in dioxane (5.68 mL, 22.7 mmol, Eq: 30) was added and the reaction stirred at 70° C. for 1 hour. The mixture was further diluted with dioxane and concentrated in vacuo. The compound 4-((3-(2,3-difluoro-4-methoxyphenyl)imidazo[1,2-a]pyrazin-8-yl)amino)-2-fluoro-6-methylbenzoic acid hydrochloride (410 mg, 750 μmol, 99% yield) was obtained as light brown solid and was used without further purification. MS ESI (m/z): 429.2 [M+H]⁺

### Step 4: tert-Butyl N-[2-[2-[[4-[[3-(2,3-difluoro-4-methoxy-phenyl)imidazo[1,2-a]pyrazin-8-yl]amino]-2-fluoro-6-methyl-benzoyl]amino]ethoxy]ethyl]carbamate

To a solution of 4-((3-(2,3-difluoro-4-methoxyphenyl)imidazo[1,2-a]pyrazin-8-yl)amino)-2-fluoro-6-methylbenzoic acid hydrochloride (20 mg, 40.4 μmol, Eq: 1) and DIPEA (20.9 mg, 28.3 μl, 162 μmol, Eq: 4) in dry DMF (207 μl) was added HATU (15.4 mg, 40.4 μmol, Eq: 1) and the mixture stirred 10 minutes at room temperature (thick suspension). Then tert-butyl (2-(2-aminoethoxy)ethyl)carbamate hydrochloride (14.6 mg, 60.7 μmol, Eq: 1.5) was added, the reaction diluted with dry DMF (104 μl) and the mixture stirred at room temperature for 1 hour. Then the reaction was concentrated in vacuo. The crude material was purified by silica gel chromatography using dichloromethane/methanol as eluent. The compound tert-butyl (2-(2-(4-((3-(2,3-difluoro-4-methoxyphenyl)imidazo[1,2-a]pyrazin-8-yl)amino)-2-fluoro-6-methylbenzamido)ethoxy)ethyl)carbamate (0.031 g, 40.3 μmol, 99.8% yield) was obtained as colorless amorphous solid and was used without further purification. MS ESI (m/z): 615.4 [M+H]⁺

### Step 5: N-(2-(2-Aminoethoxy)ethyl)-4-((3-(2,3-difluoro-4-methoxyphenyl)imidazo[1,2-a]pyrazin-8-yl)amino)-2-fluoro-6-methylbenzamide Formate

To a solution of tert-butyl (2-(2-(4-((3-(2,3-difluoro-4-methoxyphenyl)imidazo[1,2-a]pyrazin-8-yl)amino)-2-fluoro-6-methylbenzamido)ethoxy)ethyl)carbamate (31 mg, 40.3 μmol, Eq: 1) in dioxane (202 μl) was added 4 M HCl in dioxane (403 μl, 1.61 mmol, Eq: 40) and the resulting mixture stirred at room temperature (suspension). The reaction was diluted with dichloromethane and basified with 0.5 mL 7 M ammonia in MeOH. Then 1 spoon amine-silica gel was added and the mixture concentrated in vacuo. The crude material (drypack on amine silica gel) was purified by amine silica gel chromatography using dichloromethane/methanol as eluent. The obtained material was further purified by preparative reversed phase HPLC (Column: Phenomenex Gemini-NX 5u 110A, 1:100 mm, dia: 30 mm) using water containing 0.1% formic acid/acetonitrile as eluent. The compound N-(2-(2-aminoethoxy)ethyl)-4-((3-(2,3-difluoro-4-methoxyphenyl)imidazo[1,2-a]pyrazin-8-yl)amino)-2-fluoro-6-methylbenzamide formate (9 mg, 16.1 μmol, 39.8% yield) was obtained as white solid. MS ESI (m/z): 515.3 [M+H]⁺, 258.2 [M+2H]²⁺

The following examples were prepared in analogy to Reference Example 456. HCl-salts were isolated in case the compounds were clean after the last step without further purification. The free base was isolated, if the compounds were clean after silica gel chromatography or when a basic eluent was used during preparative HPLC.

| | | | Color and form, | Starting |
|---|---|---|---|---|
| Ex. | Name | Structure | analytics | Materials |
| REF 457 | N-(2-(2- aminoethoxy)ethyl)-4- ((3-(2,3-difluoro-4- methoxy- phenyl)imidazo [1,2-a]pyrazin-8- yl)amino)-2,6- difluorobenzamide | 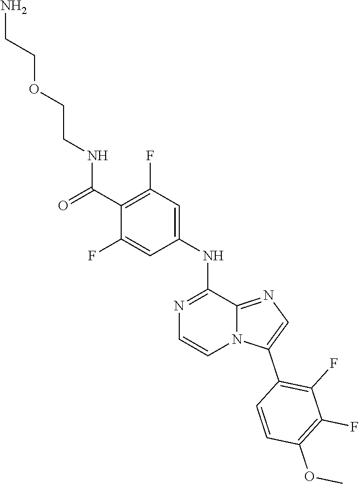 [SMILES: COc1ccc(-c2cnc3c(Nc4cc(F)c(C(=O)NCCOCCN)c(F)c4)nccn23)c(F)c1F] | White lyoph powder, MS ESI (m/z): 519.2 [M + H]⁺, 260.2 [M + 2H]²⁺ | 4-bromo- 2,6- difluoroben- zoic acid |
| REF 458 | N-(2-(2- aminoethoxy)ethyl)-2- chloro-4-((3-(2,3- difluoro-4- methoxy- phenyl)imidazo [1,2-a]pyrazin-8- yl)amino)-6- fluorobenzamide | 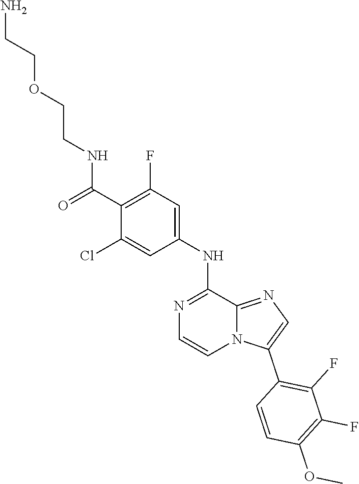 [SMILES: COc1ccc(-c2cnc3c(Nc4cc(F)c(C(=O)NCCOCCN)c(Cl)c4)nccn23)c(F)c1F] | White lyoph powder, MS ESI (m/z): 535.2, 537.2 [M + H]⁺, 268.2, 269.1 [M + 2H]²⁺ | 4-bromo-2- chloro-6- fluoroben- zoic acid |
| REF 459 | N-(2-(2- aminoethoxy)ethyl)-4- ((3-(2,3-difluroo-4- methoxy- phenyl)imidazo [1,2-a]pyrazin-8- yl)amino)-3-fluoro-2- methylbenzamide dihydrochloride | 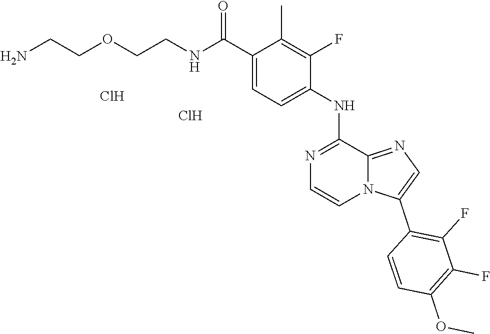 [SMILES: COc1ccc(-c2cnc3c(Nc4ccc(C(=O)NCCOCCN)c(C)c4F)nccn23)c(F)c1F ; Cl ; Cl] | Off-white solid, MS ESI (m/z): 513.3 [M − H]⁻ | 4-bromo-3- fluoro-2- methylben- zoic acid |
| REF 460 | N-(2-(2- aminoethoxy)ethyl)-2- chloro-4-((3-(2,3- difluoro-4-methoxy- phenyl)imidazo [1,2-a]pyrazin-8- yl)amino)-6- methylbenzamide | 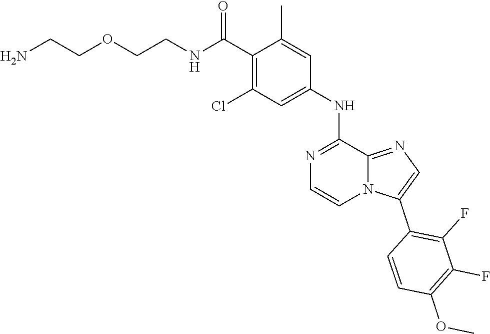 [SMILES: COc1ccc(-c2cnc3c(Nc4cc(C)c(C(=O)NCCOCCN)c(Cl)c4)nccn23)c(F)c1F] | Off-white solid, MS ESI (m/z): 531.2 [M + H]⁺ | 4-bromo-2- chloro-6- methylben- zoic acid |
| REF 461 | (4-(4-((3-(2,3-difluoro- 4-methoxy- phenyl)imidazo [1,2-a]pyrazin-8- yl)amino)-2-fluoro-6- methylbenzoyl)piper- azin-1-yl)((2S,4R)-4- hydroxypyrrolidin-2- yl)methanone dihydrochloride | 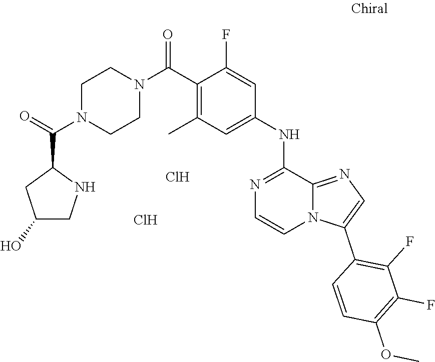 [SMILES: COc1ccc(-c2cnc3c(Nc4cc(C)c(C(=O)N5CCN(C(=O)[C@@H]6C[C@@H](O)CN6)CC5)c(F)c4)nccn23)c(F)c1F ; Cl ; Cl] | MS ESI (m/z): 610.2391 [M + H]⁺ | Intermediate 139 |
| 49 | (4-(4-((3-(2,3-difluoro- 4-methoxy- phenyl)imidazo [1,2-a]pyrazin-8- yl)amio)-2,6-difluoro- benzoyl)piperazin- 1-yl)((2S,4R)-4- hydroxypyrrolidin-2- yl)methanone dihydrochloride | 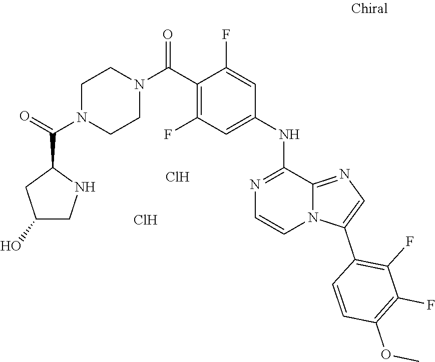 [SMILES: COc1ccc(-c2cnc3c(Nc4cc(F)c(C(=O)N5CCN(C(=O)[C@@H]6C[C@@H](O)CN6)CC5)c(F)c4)nccn23)c(F)c1F ; Cl ; Cl] | MS ESI (m/z): 614.3 [M + H]⁺, 307.7 [M + 2H]²⁺ | 4-bromo- 2,6- difluoroben- zoic acid, Intermediate 139 |
| 50 | (4-(2-chloro-4-((3- (2,3-difluoro-4- methoxyphenyl)imi- dazo[1,2-a]pyrazin-8- yl)amino)-6-fluoro- benzoyl)piperazin- 1-yl)((2S,4R)-4- hydroxypyrrolidin-2- yl)methanone dihydrochloride | 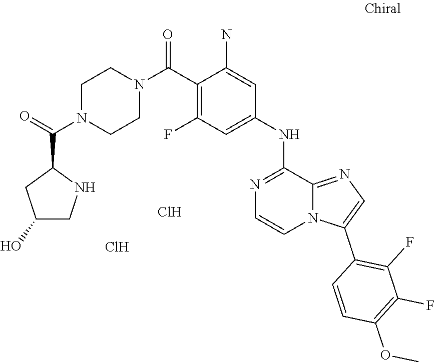 | MS ESI (m/z): 630.3, 632.4 [M + H]⁺, 315.7, 316.5 [M + 2H]²⁺ | 4-bromo-2- chloro-6- fluorobenzoic acid, Intermediate 139 |
| REF 462 | (4-(4-((3-(2,3-di- fluoro-4-methoxy- phenyl)imidazo [1,2-a]pyrazin-8- yl)amino)-2,5-di- fluorobenzoyl)piper- azin-1-yl)((2S,4R)-4- hydroxypyrrolidin-2- yl)methanone | 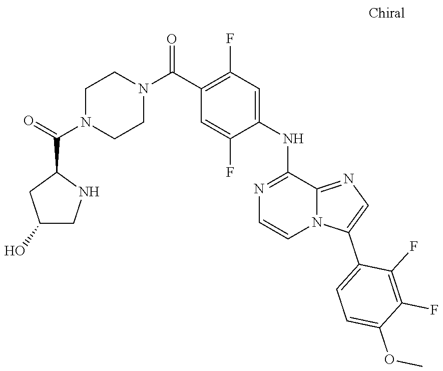 [SMILES: COc1ccc(-c2cnc3c(Nc4cc(F)c(C(=O)N5CCN(C(=O)[C@@H]6C[C@@H](O)CN6)CC5)cc4F)nccn23)c(F)c1F] | MS ESI (m/z): 614.3 [M + H]⁺, 307.7 [M + 2H]²⁺ | 4-bromo- 2,5- difluoroben- zoic acid, Intermediate 139 |
| REF 463 | N-(2-(2-amino- ethoxy)ethyl)-4- ((3-(2,3-difluoro-4- methoxyphenyl)imi- dazo[1,2-a]pyrazin-8- yl)amino)-2,3- difluorobenzamide | 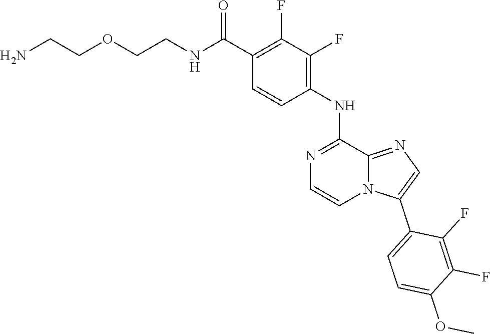 [SMILES: COc1ccc(-c2cnc3c(Nc4ccc(C(=O)NCCOCCN)c(F)c4F)nccn23)c(F)c1F] | MS ESI (m/z): 519.3 [M + H]⁺, 260.2 [M + 2H]²⁺ | 4-bromo- 2,3- difluoroben- zoic acid |

### Reference Example 464

### N-(2-((2-Aminoethyl)amino)ethyl)-4-((3-(2,3-difluoro-4-methoxyphenyl)imidazo[1,2-a]pyrazin-8-yl)amino)-2-ethylbenzamide

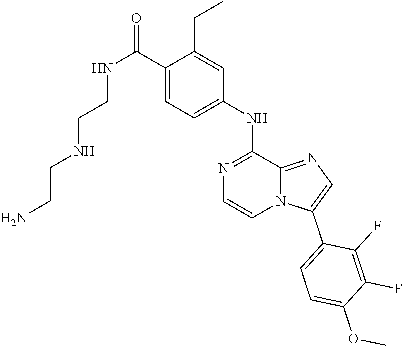 [SMILES: CCc1cc(Nc2nccn3c(-c4ccc(OC)c(F)c4F)cnc23)ccc1C(=O)NCCNCCN]

To a solution of 4-((3-(2,3-difluoro-4-methoxyphenyl)imidazo[1,2-a]pyrazin-8-yl)amino)-2-ethylbenzoic acid hydrochloride (Example 86, 100 mg, 217 μmol, Eq: 1) and DIPEA (112 mg, 152 μl, 868 μmol, Eq: 4) in dry DMF (1.08 mL) was added HATU (107 mg, 282 μmol, Eq: 1.3) and the mixture was shaken for 15 minutes at room temperature. Then N1-(2-aminoethyl)ethane-1,2-diamine (89.5 mg, 93.8 μl, 868 μmol, Eq: 4) was added and shaking continued at room temperature for 3 hours. Then the reaction mixture was concentrated in vacuo. The material was purified by preparative reversed phase HPLC (Column: YMC Actus Triart C18 5 um, 1:100 mm, dia: 30 mm) using water containing 0.1% triethylamine/acetonitrile as eluent. The compound N-(2-((2-aminoethyl)amino)ethyl)-4-((3-(2,3-difluoro-4-methoxyphenyl)imidazo[1,2-a]pyrazin-8-yl)amino)-2-ethylbenzamide (39 mg, 74.2 μmol, 34.2% yield) was obtained as white solid. MS ESI (m/z): 510.2433 [M+H]⁺

The following examples were prepared in analogy to Reference Example 464.

| | | | | Starting |
|---|---|---|---|---|
| Ex. | Name | Structure | Color and form, MS | Materials |
| REF 465 | rac-N-((1R,2S)-2- aminocyclohexyl)-4- ((3-(2,3-difluoro-4- methoxyphenyl)imidazo [1,2-a]pyrazin-8- yl)amino)-2- ethylbenzamide | 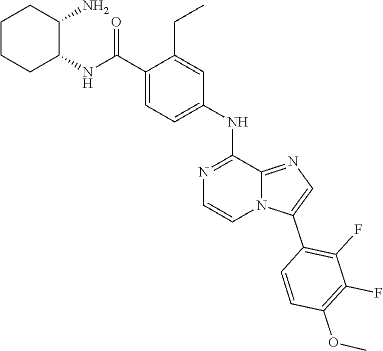 [SMILES: CCc1cc(Nc2nccn3c(-c4ccc(OC)c(F)c4F)cnc23)ccc1C(=O)N[C@@H]1CCCC[C@@H]1N] | Brown solid, MS ESI (m/z): 521.2 [M + H]⁺ | rac- (1R,2S)- cyclohexane- 1,2- diamine |
| REF 466 | N-((1S,2S)-2- aminocyclopentyl)-4- ((3-(2,3-difluoro-4- methoxyphenyl)imidazo [1,2-a]pyrazin-8- yl)amino)-2- ethylbenzamide | 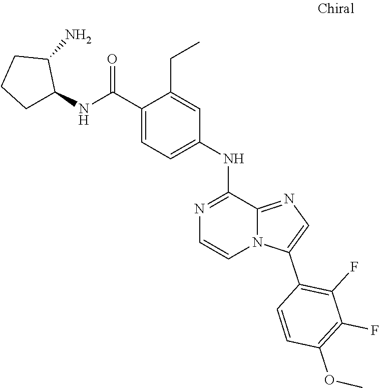 [SMILES: CCc1cc(Nc2nccn3c(-c4ccc(OC)c(F)c4F)cnc23)ccc1C(=O)N[C@H]1CCC[C@@H]1N] | White solid, MS ESI (m/z) 507.3 [M + H]⁺ | (1S,2S)- cyclopentane- 1,2- diamine dihydrochlo- ride |

### Reference Example 467

### N-(2-(2-(2-Aminoethoxy)ethoxy)ethyl)-4-((3-(2,3-difluoro-4-methoxyphenyl)imidazo[1,2-a]pyrazin-8-yl)amino)-2-ethylbenzamide Dihydrochloride

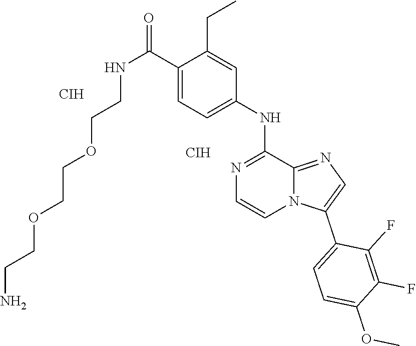 [SMILES: CCc1cc(Nc2nccn3c(-c4ccc(OC)c(F)c4F)cnc23)ccc1C(=O)NCCOCCOCCN ; "CIH" ; "CIH"]

### Step 1: tert-Butyl (2-(2-(2-(4-((3-(2,3-difluoro-4-methoxyphenyl)imidazo[1,2-a]pyrazin-8-yl)amino)-2-ethylbenzamido)ethoxy)ethoxy)ethyl)carbamate

To a solution of 4-((3-(2,3-difluoro-4-methoxyphenyl)imidazo[1,2-a]pyrazin-8-yl)amino)-2-ethylbenzoic acid hydrochloride (Example 86, 40 mg, 86.8 μmol, Eq: 1) and DIPEA (44.9 mg, 60.6 μl, 347 μmol, Eq: 4) in dry DMF (434 μl) was added HATU (36.3 mg, 95.5 μmol, Eq: 1.1) and the mixture was shaken for 10 minutes at room temperature.

Then tert-butyl (2-(2-(2-aminoethoxy)ethoxy)ethyl)carbamate (28 mg, 113 μmol, Eq: 1.3) was added and shaking continued at room temperature for 4 hours. The reaction mixture was diluted with ethyl acetate, sat. aqueous NaHCO₃ solution and brine. The mixture was extracted 2× with ethyl acetate and the organic layers were washed 2× with brine. The combined organic layers were dried with sodium sulfate, filtered and concentrated in vacuo. The crude material was purified by silica gel chromatography using dichloromethane/methanol as eluent. The compound tert-butyl (2-(2-(2-(4-((3-(2,3-difluoro-4-methoxyphenyl)imidazo[1,2-a]pyrazin-8-yl)amino)-2-ethylbenzamido)ethoxy)ethoxy)ethyl)carbamate (41.1 mg, 62.8 μmol, 72.3% yield) was obtained as off-white solid. MS ESI (m/z): 655.4 [M+H]⁺

### Step 2: N-(2-(2-(2-aminoethoxy)ethoxy)ethyl)-4-((3-(2,3-difluoro-4-methoxyphenyl)imidazo[1,2-a]pyrazin-8-yl)amino)-2-ethylbenzamide Dihydrochloride

In a 5 mL round-bottomed flask, tert-butyl (2-(2-(2-(4-((3-(2,3-difluoro-4-methoxyphenyl)imidazo[1,2-a]pyrazin-8-yl)amino)-2-ethylbenzamido)ethoxy)ethoxy)ethyl)carbamate (41.4 mg, 63.2 μmol, Eq: 1) and 4 M HCl in dioxane (632 μl, 2.53 mmol, Eq: 40) were combined to give a light yellow solution. The reaction mixture was stirred at room temperature for 3 hours. The reaction was diluted with water and directly lyophilized. The compound N-(2-(2-(2-aminoethoxy)ethoxy)ethyl)-4-((3-(2,3-difluoro-4-methoxyphenyl)imidazo[1,2-a]pyrazin-8-yl)amino)-2-ethylbenzamide dihydrochloride (37.3 mg, 59.4 μmol, 94% yield) was obtained as yellow solid. MS ESI (m/z): 278.3 [M+2H]²⁺

The following examples were prepared in analogy to Reference Example 467. In case the free base was isolated, the obtained material was further purified by silica gel chromatography and/or preparative HPLC.

| | | | | Starting |
|---|---|---|---|---|
| Ex. | Name | Structure | Color and form, MS | Materials |
| REF 468 | N-((1S,2R)-2- aminocyclopentyl)-4- ((3-(2,3-difluoro-4- methoxyphenyl)imidazo [1,2-a]pyrazin-8- yl)amino)-2- ethylbenzamide | 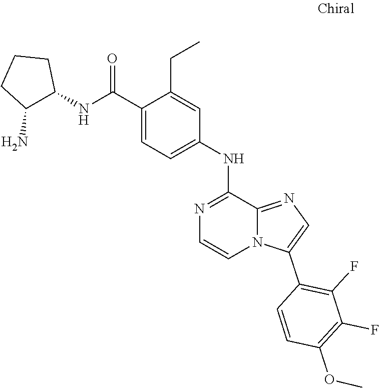 [SMILES: CCc1cc(Nc2nccn3c(-c4ccc(OC)c(F)c4F)cnc23)ccc1C(=O)N[C@H]1CCC[C@H]1N] | Light brown solid, MS ESI (m/z): 507.3 [M + H]⁺, 254.3 [M + 2H]²⁺ | tert-butyl ((1R,2S)-2- aminocyclo- pentyl)car- bamate |

### Reference Example 469

### 4-((3-(2,3-difluoro-4-methoxyphenyl)imidazo[1,2-a]pyrazin-8-yl)amino)-2-ethyl-N-(2-(2-(methyl(2-(methylsulfonamido)-2-oxoethyl)amino)ethoxy)ethyl)benzamide Dihydrochloride

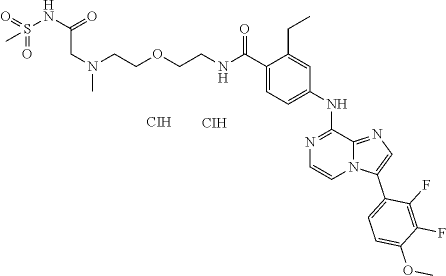 [SMILES: CCc1cc(Nc2nccn3c(-c4ccc(OC)c(F)c4F)cnc23)ccc1C(=O)NCCOCCN(C)CC(=O)NS(C)(=O)=O ; "CIH" ; "CIH"]

To a solution of N-(2-(2-(4-((3-(2,3-difluoro-4-methoxyphenyl)imidazo[1,2-a]pyrazin-8-yl)amino)-2-ethylbenzamido)ethoxy)ethyl)-N-methylglycine hydrochloride (Reference Example 449, 20 mg, 32.3 μmol, Eq: 1), methanesulfonamide (3.99 mg, 42 μmol, Eq: 1.3) and DMAP (5.13 mg, 42 μmol, Eq: 1.3) in dry dichloromethane (215 μl) was added 1-(3-dimethylaminopropyl)-3-ethylcarbodiimide (6.52 mg, 7.43 μL, 42 μmol, Eq: 1.3) and the mixture stirred at room temperature for 18 hours. Then the reaction was concentrated in vacuo.

The crude material was purified by preparative reversed phase HPLC (Column: YMC Actus Triart C18, 12 nm, 5 μm, 1:100 mm, dia: 30 mm) using acetonitrile/water containing 0.1% formic acid as eluent. The obtained solution was lyophilized. The residue was redisolved in 0.1M aq. HCl and again lyophilized.

The compound 4-((3-(2,3-difluoro-4-methoxyphenyl)imidazo[1,2-a]pyrazin-8-yl)amino)-2-ethyl-N-(2-(2-(methyl(2-(methylsulfonamido)-2-oxoethyl)amino)ethoxy)ethyl)benzamide dihydrochloride (16.5 mg, 22.5 μmol, 69.7% yield) was obtained as light yellow solid. MS ESI (m/z): 660.2417 [M+H]⁺

### Reference Example 470

### (4S)-4-Amino-5-((1-((3-(4-((3-(2,3-difluoro-4-methoxyphenyl)imidazo[1,2-a]pyrazin-8-yl)amino)-2-ethylbenzamido)propyl)amino)-3-guanidino-1-oxopropan-2-yl)amino)-5-oxopentanoic acid trihydrochloride (Epimers 1:1)

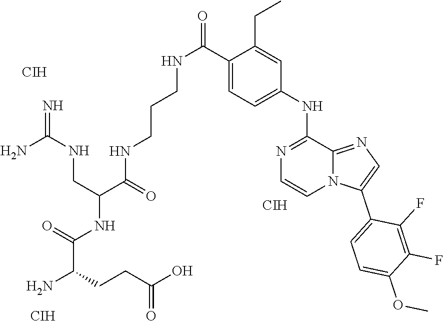

### Step 1: Boc-Glu(OtBu)-Agp(Boc)₂-OH

A solution of Fmoc-Agp(Boc)₂-OH (426 mg, 750 μmol, Eq: 0.625) and DIPEA biotech grade (775 mg, 1.05 mL, 6 mmol, Eq: 5) in dry dichloromethane (6.5 mL) was added to 2-chlorotrityl chloride-resin (Bachem, 1.6 mmol/g, 0.75 g, 1.2 mmol, Eq: 1) in a dried glass bottle and put under argon. The reaction mixture was shaken under argon atmosphere for 16 hours at room temperature. To the mixture was added methanol (596 μl) (0.8 mL per gram resin) and the reaction mixture was shaken for 4 hours at room temperature to cap the remaining chloride. The mixture was filtered and then washed 3× with 5 mL dichloromethane, followed by 3×5 mL DMF. 4-methylpiperidine/DMF/DCM 2:1:1 (4.65 mL) was added to the resin. The reaction mixture was shaken for 30 minutes at room temperature. The resin was filtered and washed 2× with 5 mL DCM and 2× with 5 mL DMF. Again 4-methylpiperidine/DMF/DCM 2:1:1 (4.65 mL) was added to the resin and the mixture shaken for 30 minutes at room temperature. The resin was filtered and washed 2× with 5 mL DCM and 2× with 2 mL DMF. On the side a solution of Boc-Glu(OtBu)—OH (455 mg, 1.5 mmol, Eq: 1.25) and DIPEA (388 mg, 524 μl, 3 mmol, Eq: 2.5) in DMF/DCM 1:1 (4.65 mL) was treated with HATU (570 mg, 1.5 mmol, Eq: 1.25) and stirred for 10 minutes. The resulting mixture was added to the resin and shaken for 18 hours. The resin was filtered and washed 3× with 5 mL DMF and 3× with 5 mL DCM. Then the resin was treated with 5 mL DCM/HFIP 4:1 and shaken 1 hour. The mixture was filtered and washed 3× with DCM. This cleavage procedure was repeated 1 more time. The obtained filtrates were combined and concentrated in vacuo. The obtained oil was redisolved in acetonitrile/water and was lyophilized. The compound Boc-Glu(OtBu)-Agp(Boc)2-OH (156 mg, 247 μmol, 32.9% yield) was obtained as light brown lyoph powder and was used without further purification. MS ESI (m/z): 632.5 [M+H]⁺

### Step 2: tert-butyl (12S,E)-6,12-bis((tert-butoxycarbonyl)amino)-9-((3-(4-((3-(2,3-difluoro-4-methoxyphenyl)imidazo[1,2-a]pyrazin-8-yl)amino)-2-ethylbenzamido)propyl)carbamoyl)-2,2-dimethyl-4,11-dioxo-3-oxa-5,7,10-triazapentadec-5-en-15-oate (Epimers 1:1)

To a solution of Boc-Glu(OtBu)-Agp(Boc)2-OH (74.2 mg, 117 μmol, Eq: 1.3) and DIPEA (46.7 mg, 63.1 μl, 361 μmol, Eq: 4) in dry DMF (452 μl) was added HATU (44.7 mg, 117 μmol, Eq: 1.3) and the mixture stirred 10 minutes at room temperature. Then N-(3-aminopropyl)-4-((3-(2,3-difluoro-4-methoxyphenyl)imidazo[1,2-a]pyrazin-8-yl)amino)-2-ethylbenzamide dihydrochloride (Reference Example 297, 50 mg, 90.3 μmol, Eq: 1) was added and stirring at room temperature continued for 1.5 hours, then stored in the fridge for 16 hours. The reaction was concentrated in vacuo at 45° C. The crude material was purified by silica gel chromatography using dichloromethane/methanol as eluent. The obtained material was further purified by preparative reversed phase HPLC (Column: Phenomenex Gemini-NX 5u 110A, 1:100 mm, dia: 30 mm) using acetonitrile/water containing 0.1% triethylamine as eluent.

The compound tert-butyl (12S,E)-6,12-bis((tert-butoxycarbonyl)amino)-9-((3-(4-((3-(2,3-difluoro-4-methoxyphenyl)imidazo[1,2-a]pyrazin-8-yl)amino)-2-ethylbenzamido)propyl)carbamoyl)-2,2-dimethyl-4,11-dioxo-3-oxa-5,7,10-triazapentadec-5-en-15-oate (Epimers 1:1, 0.035 g, 30.7 μmol, 34% yield) was obtained as off-white amorphous with a purity of 96% (total UV, 210-400 nm). MS ESI (m/z): 1080.5 [M+H]⁺

### Step 3: (4S)-4-amino-5-((1-((3-(4-((3-(2,3-difluoro-4-methoxyphenyl)imidazo[1,2-a]pyrazin-8-yl)amino)-2-ethylbenzamido)propyl)amino)-3-guanidino-1-oxopropan-2-yl)amino)-5-oxopentanoic Acid Trihydrochloride (Epimers 1:1)

To a solution of tert-butyl (12S,E)-6,12-bis((tert-butoxycarbonyl)amino)-9-((3-(4-((3-(2,3-difluoro-4-methoxyphenyl)imidazo[1,2-a]pyrazin-8-yl)amino)-2-ethylbenzamido)propyl)carbamoyl)-2,2-dimethyl-4,11-dioxo-3-oxa-5,7,10-triazapentadec-5-en-15-oate (0.035 g, 32 μmol, Eq: 1) in dioxane (107 μL) was added 4M HCl in dioxane (480 μL, 1.92 mmol, Eq: 60) and the mixture stirred 16 hours at room temperature. Then the reaction was diluted with more dioxane and directly lyophilized. The compound (4)-4-amino-5-((1-((3-(4-((3-(2,3-difluoro-4-methoxyphenyl)imidazo[1,2-a]pyrazin-8-yl)amino)-2-ethylbenzamido)propyl)amino)-3-guanidino-1-oxopropan-2-yl)amino)-5-oxopentanoic acid trihydrochloride (30 mg, 29.7 μmol, 93% yield) was obtained as white lyoph powder with a purity of 84% (960 by total UV (210-400 nm), 13% dioxane acc to NM/R). MS ESI (m/z): 738.4 [M+H]⁺]

The following examples were prepared in analogy to Reference Example 470.

| | | | Color and | |
|---|---|---|---|---|
| | | | form, | Starting |
| Ex. | Name | Structure | analytics | Materials |
| REF 471 | (S)-4-amino-5-(((S)- 1-((3-(4-((3-(2,3- difluoro-4-methoxy- phenyl)imida- zo[1,2-a]pyrazin-8- yl)amino)-2-ethyl- benzamido)propyl) amino)-5-guanidino- 1-oxopentan-2- yl)amino)-5- oxopentanoic acid trihydrochloride | 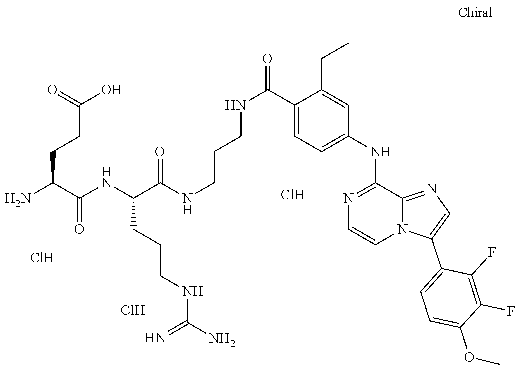 [SMILES: CCc1cc(Nc2nccn3c(-c4ccc(OC)c(F)c4F)cnc23)ccc1C(=O)NCCCNC(=O)[C@H](CCCNC(=N)N)NC(=O)[C@@H](N)CCC(=O)O ; Cl ; Cl ; Cl] | Off-white lyophilized powder, MS ESI (m/z): 766.4 [M + H]⁺ | Fmoc- Arg/(Boc)₂-OH, Boc-Glu-OtBu |

### Reference Example 472

### (S)-7-amino-1-(4-((3-(2,3-difluoro-4-methoxyphenyl)imidazo[1,2-a]pyrazin-8-yl)amino)-2-ethylphenyl)-12-imino-1,6-dioxo-2,5,11,13-tetraazaheptadecan-17-oic Acid Trihydrochloride

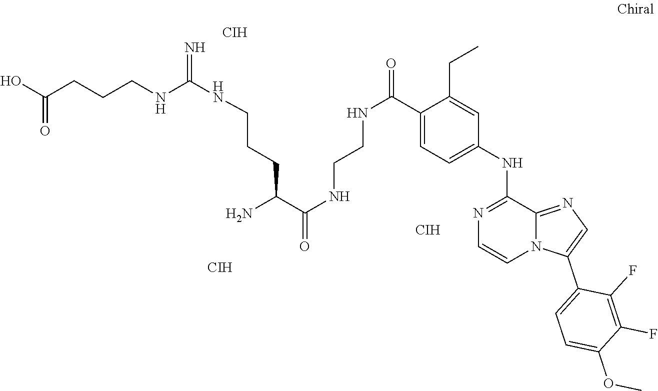

### Step 1: benzyl tert-butyl (5-((2-(4-((3-(2,3-difluoro-4-methoxyphenyl)imidazo[1,2-a]pyrazin-8-yl)amino)-2-ethylbenzamido)ethyl)amino)-5-oxopentane-1,4-diyl)(S)-dicarbamate

To a solution of Boc-Orn(Z)—OH (112 mg, 306 μmol, Eq: 1.1) in dry DMF (1.39 mL) and DIPEA (180 mg, 243 μL, 1.39 mmol, Eq: 5) was added HATU (116 mg, 306 μmol, Eq: 1.1) and the mixture stirred 10 minutes at room temperature. Then N-(2-aminoethyl)-4-((3-(2,3-difluoro-4-methoxyphenyl)imidazo[1,2-a]pyrazin-8-yl)amino)-2-ethylbenzamide dihydrochloride (Reference Example 296, 0.15 g, 278 μmol, Eq: 1) was added and stirring at room temperature continued for 2 hours. Then the reaction mixture was diluted with ethyl acetate, water and sat. aqueous NaHCO₃ solution. The mixture was extracted 2× with ethyl acetate and the organic layers were washed 2× with brine. The combined organic layers were dried with sodium sulfate, filtered and concentrated in vacuo. The crude material was purified by silica gel chromatography using dichloromethane/methanol as eluent. The compound benzyl tert-butyl (5-((2-(4-((3-(2,3-difluoro-4-methoxyphenyl)imidazo[1,2-a]pyrazin-8-yl)amino)-2-ethylbenzamido)ethyl)amino)-5-oxopentane-1,4-diyl)(S)-dicarbamate (0.207 g, 254 μmol, 91.3% yield) was obtained as light brown amorphous solid. MS ESI (m/z): 815.6 [M+H]⁺

### Step 2: tert-butyl (S)-(5-amino-1-((2-(4-((3-(2,3-difluoro-4-methoxyphenyl)imidazo[1,2-a]pyrazin-8-yl)amino)-2-ethylbenzamido)ethyl)amino)-1-oxopentan-2-yl)

A suspension of benzyl tert-butyl (5-((2-(4-((3-(2,3-difluoro-4-methoxyphenyl)imidazo[1,2-a]pyrazin-8-yl)amino)-2-ethylbenzamido)ethyl)amino)-5-oxopentane-1,4-diyl)(S)-dicarbamate (0.207 g, 254 μmol, Eq: 1) in methanol (2.54 mL) was evacuated 3× (frothing) and flushed with argon. Then 10% palladium on charcoal (27 mg, 25.4 μmol, Eq: 0.1) was added and degassing repeated. Then again the mixture was evacuated 3× (frothing) and flushed with hydrogen. The reaction was stirred 3 hours at room temperature under hydrogen. Then the reaction was diluted with ethanol (2.54 mL) and stirring under hydrogen continued for another 20 hours. The reaction mixture was filtered and washed with EtOH and dichloromethane/MeOH 9:1. The obtained solution was concentrated in vacuo. The compound tert-butyl (S)-(5-amino-1-((2-(4-((3-(2,3-difluoro-4-methoxyphenyl)imidazo[1,2-a]pyrazin-8-yl)amino)-2-ethylbenzamido)ethyl)amino)-1-oxopentan-2-yl)carbamate (162 mg, 238 μmol, 93.7% yield) was obtained as light brown amorphous solid and was used without further purification. MS ESI (m/z): 681.5 [M+H]⁺

### Step 3: tert-butyl (S,Z)-13-(tert-butoxycarbonyl)-7-((tert-butoxycarbonyl)amino)-12-((tert-butoxycarbonyl)imino)-1-(4-((3-(2,3-difluoro-4-methoxyphenyl)imidazo[1,2-a]pyrazin-8-yl)amino)-2-ethylphenyl)-1,6-dioxo-2,5,11,13-tetraazaheptadecan-17-oate

A solution of tert-butyl (E)-4-(N,N′-bis(tert-butoxycarbonyl)-1H-pyrazole-1-carboximidamido)butanoate (Intermediate 141, 21.4 mg, 44.1 μmol, Eq: 1) in acetonitrile (294 μl) was added to tert-butyl (S)-(5-amino-1-((2-(4-((3-(2,3-difluoro-4-methoxyphenyl)imidazo[1,2-a]pyrazin-8-yl)amino)-2-ethylbenzamido)ethyl)amino)-1-oxopentan-2-yl)carbamate (30 mg, 44.1 μmol, Eq: 1). To the resulting suspension DIPEA (12.5 mg, 16.9 μL, 97 μmol, Eq: 2.2) was added and the reaction stirred 16 hours at room temperature. Again tert-butyl (E)-4-(N,N′-bis(tert-butoxycarbonyl)-1H-pyrazole-1-carboximidamido)butanoate (Intermediate 141, 9.97 mg, 22 μmol, Eq: 0.5) in acetonitrile (147 μl) was added and stirring at room temperature continued. Then the reaction was concentrated in vacuo. The crude material was purified by silica gel chromatography using heptane/(ethyl acetate/EtOH/NH4OH 75:25:2) as eluent. The compound tert-butyl (S,Z)-13-(tert-butoxycarbonyl)-7-((tert-butoxycarbonyl)amino)-12-((tert-butoxycarbonyl)imino)-1-(4-((3-(2,3-difluoro-4-methoxyphenyl)imidazo[1,2-a]pyrazin-8-yl)amino)-2-ethylphenyl)-1,6-dioxo-2,5,11,13-tetraazaheptadecan-17-oate (0.014 g, 13.1 μmol, 29.8% yield) was obtained as colorless amorphous solid and was used without further purification. MS ESI (m/z): 1065.6 [M+H]⁺

### Step 4: (S)-7-amino-1-(4-((3-(2,3-difluoro-4-methoxyphenyl)imidazo[1,2-a]pyrazin-8-yl)amino)-2-ethylphenyl)-12-imino-1,6-dioxo-2,5,11,13-tetraazaheptadecan-17-oic Acid Trihydrochloride

To a suspension of tert-butyl (S,Z)-13-(tert-butoxycarbonyl)-7-((tert-butoxycarbonyl)amino)-12-((tert-butoxycarbonyl)imino)-1-(4-((3-(2,3-difluoro-4-methoxyphenyl)imidazo[1,2-a]pyrazin-8-yl)amino)-2-ethylphenyl)-1,6-dioxo-2,5,11,13-tetraazaheptadecan-17-oate (13 mg, 12.2 μmol, Eq: 1) in dioxane (40.7 μL) was added 4M HCl in dioxane (305 μL, 1.22 mmol, Eq: 100) and the resulting solution stirred at room temperature for 4 hours. The reaction was diluted with dioxane and directly lyophilized. The compound (S)-7-amino-1-(4-((3-(2,3-difluoro-4-methoxyphenyl)imidazo[1,2-a]pyrazin-8-yl)amino)-2-ethylphenyl)-12-imino-1,6-dioxo-2,5,11,13-tetraazaheptadecan-17-oic acid trihydrochloride (7 mg, 8.13 μmol, 66.6% yield) was obtained as white lyoph powder with a purity of 95% (UV, 265 nm). MS ESI (m/z): 709.3379 [M+H]⁺

### Reference Example 473

### Cis N-(3-aminocyclobutyl)-4-[[3-(2,3-difluoro-4-methoxy-phenyl)imidazo[1,2-a]pyrazin-8-yl]amino]-2-ethyl-benzamide

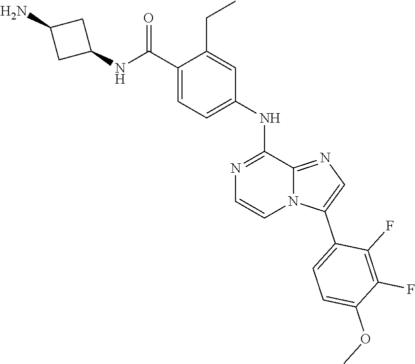 [SMILES: CCc1cc(Nc2nccn3c(-c4ccc(OC)c(F)c4F)cnc23)ccc1C(=O)N[C@H]1C[C@@H](N)C1]

### Step 1

### Cis-tert-butyl N-[3-[[4-[[3-(2,3-difluoro-4-methoxy-phenyl)imidazo[1,2-a]pyrazin-8-yl]amino]-2-ethyl-benzoyl]amino]cyclobutyl]carbamate

Under Ar, 4-((3-(2,3-difluoro-4-methoxyphenyl)imidazo[1,2-a]pyrazin-8-yl)amino)-2-ethylbenzoic acid, Intermediate 86 (100 mg, 236 μmol, Eq: 1) was suspended in DMF (1 mL). tert-Butyl cis-N-(3-aminocyclobutyl)carbamate [CAS #1212395-34-0] (1.18 mmol, Eq: 5) was added. Additional tert-Butyl cis-N-(3-aminocyclobutyl)carbamate [CAS #1212395-34-0] (283 μmol, Eq: 1.2) and 2-(3H-[1,2,3]triazolo[4,5-b]pyridin-3-yl)-1,1,3,3-tetramethylisouronium hexafluorophosphate(V) (HATU) (179 mg, 471 μmol, Eq: 2) were added and the yellow solution was stirred at RT over 2 h. The solvent was evaporated and the crude material was purified by flash chromatography (silica gel, 20 g, 0% to 100% DCM/MeOH/NH₄₀H (95/5/1)) leading to the target compound (orange foam, 140 mg). MS (ESI, m/z): 593.3 [M+H]⁺.

### Step 2

### Cis N-(3-aminocyclobutyl)-4-[[3-(2,3-difluoro-4-methoxy-phenyl)imidazo[1,2-a]pyrazin-8-yl]amino]-2-ethyl-benzamide

Under Ar, cis-tert-butyl N-[3-[[4-[[3-(2,3-difluoro-4-methoxy-phenyl)imidazo[1,2-a]pyrazin-8-yl]amino]-2-ethyl-benzoyl]amino]cyclobutyl]carbamate obtained in step 1 (140 mg) was dissolved in MeOH (1 mL). 4M HCl in dioxane (1.07 mL, 4.27 mmol, Eq: 10) was added and the RM was stirred at RT over 3 h. DCM/MeOH/aq. NH₃ was added till the HCl was neutralized and the RM was evaporated. The crude product was purified by flash chromatography (silica gel, 20 g, 0% to 100% DCM/MeOH/aq. 25% NH₄OH (90/10/1)) leading to the title compound (white solid, 103 mg). MS (ESI, m/z): 493.2 [M+H]+.

The following examples were prepared in analogy to Reference Example 473.

| | | | ESI | |
|---|---|---|---|---|
| | | | MS | |
| Ex. | Name | Structure | [M + H]+ | Starting Materials |
| REF 474 | N-(azetidin-3-yl)-4-[[3- (2,3-difluoro-4- methoxy- phenyl)imidazo[1,2- a]pyrazin-8-yl]amino]- 2-ethyl-benzamide | 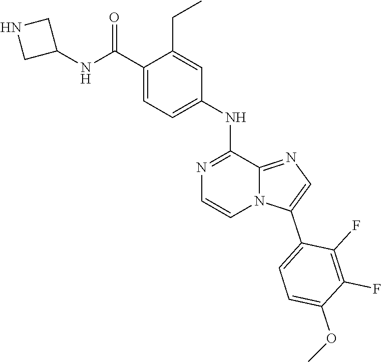 [SMILES: CCc1cc(Nc2nccn3c(-c4ccc(OC)c(F)c4F)cnc23)ccc1C(=O)NC1CNC1] | 479.3 | Intermediate 86 and tert-butyl 3- aminoazetidine-1- carboxylate. Deprotection with TFA/DCM 1:2 at rt for 1 h. |
| REF 475 | 4-[[3-(2,3-difluroo-4- methoxy- phenyl)imidazo[1,2- a]pryazin-8-yl]amino]- 2-ethyl-N-(4- piperidyl)benzamide | 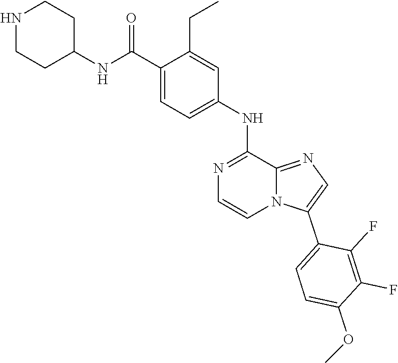 [SMILES: CCc1cc(Nc2nccn3c(-c4ccc(OC)c(F)c4F)cnc23)ccc1C(=O)NC1CCNCC1] | 507.3 | Intermediate 86 and tert-butyl 4- aminopiperidine-1- carboxylate |
| REF 426 | Cis-N-(4- aminocyclohexyl)-4- [[3-(2,3-difluoro-4- methoxy- phenyl)imidazo[1,2- a]pyrazin-8-yl]amino]- 2-ethyl-benzamide | 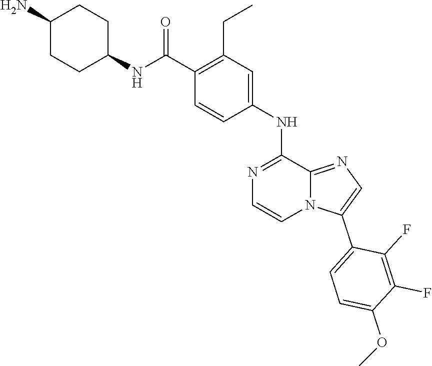 [SMILES: CCc1cc(Nc2nccn3c(-c4ccc(OC)c(F)c4F)cnc23)ccc1C(=O)N[C@H]1CC[C@@H](N)CC1] | 521.3 | Intermediate 86 and cis-tert-butyl (-4- aminocyclohexyl)car- bamate |
| REF 476 | N-(3-aminocyclobutyl)- 4-[[3-(2,3-difluoro-4- methoxy- phenyl)imidazo[1,2- a]pyrazin-8-yl]amino]- 2-ethyl-benzamide | 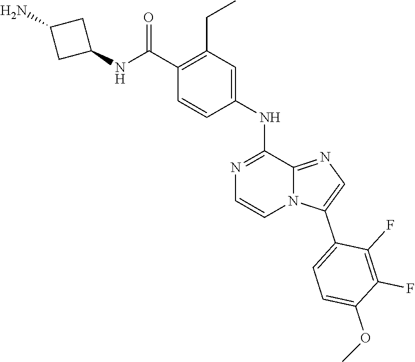 [SMILES: CCc1cc(Nc2nccn3c(-c4ccc(OC)c(F)c4F)cnc23)ccc1C(=O)N[C@H]1C[C@H](N)C1] | 493.3 | Intermediate 86 and trans-tert-butyl (3- aminocyclobutyl)car- bamate |
| 128 | N-(3-amino-2-hydroxy- propyl)-4-[[3-(2,3- difluoro-4-methoxy- phenyl)imidazo[1,2- a]pyrazin-8-yl]amino]- 2-ethyl- benzamide; hydrochloride | 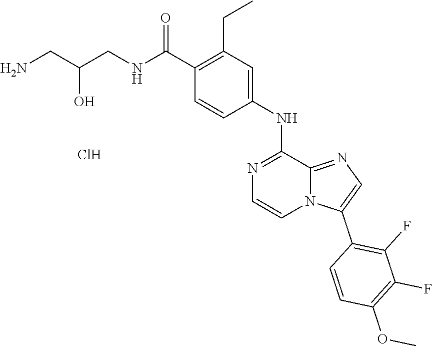 [SMILES: CCc1cc(Nc2nccn3c(-c4ccc(OC)c(F)c4F)cnc23)ccc1C(=O)NCC(O)CN ; Cl] | 495.4 | 86 and tert-butyl (3- amino-2- hydroxypropyl)carba- mate [CAS#144912-84- 5]. Product isolated by filtration of reaction mixture following deprotection step. |

### Reference Example 477

### 4-[4-[[3-[4-(difluoromethoxy)phenyl]imidazo[1,2-a]pyrazin-8-yl]amino]-2-methyl-benzoyl]-N-(4-pyridyl)piperazine-1-carboxamide

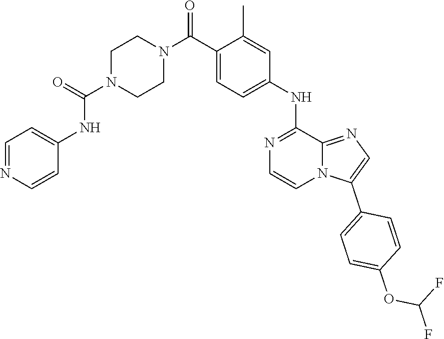 [SMILES: Cc1cc(Nc2nccn3c(-c4ccc(OC(F)F)cc4)cnc23)ccc1C(=O)N1CCN(C(=O)Nc2ccncc2)CC1]

To a solution of [4-[[3-[4-(difluoromethoxy)phenyl]imidazo[1,2-a]pyrazin-8-yl]amino]-2-methyl-phenyl]-piperazin-1-yl-methanone hydrochloride (Reference Example 294) (100.0 mg, 0.210 mmol, 1 eq) in DMF (5 mL) was added phenyl N-(4-pyridyl)carbamate (67.16 mg, 0.310 mmol, 1.5 eq) and N,N-diisopropylethylamine (0.11 mL, 0.630 mmol, 3 eq), then the reaction was stirred at 25° C. for 12 h. The reaction mixture was purified by prep-HPLC (basic) to give product 4-[4-[[3-[4-(difluoromethoxy)phenyl]imidazo[1,2-a]pyrazin-8-yl]amino]-2-methyl-benzoyl]-N-(4-pyridyl)piperazine-1-carboxamide (30.5 mg, 0.050 mmol, 24% yield) as a yellow solid. MS (ESI, m/z): 599.1 [M+H]+.

### Reference Example 478

### N-[2-(2-aminoethoxy)ethyl]-4-[[3-(4-but-2-ynoxyphenyl)imidazo[1,2-a]pyrazin-8-yl]amino]-2-methyl-benzamide; 2,2,2-trifluoroacetic Acid

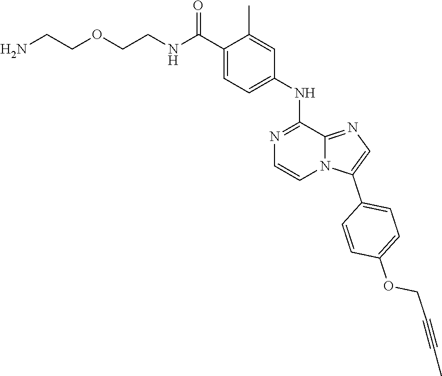 [SMILES: CC#CCOc1ccc(-c2cnc3c(Nc4ccc(C(=O)NCCOCCN)c(C)c4)nccn23)cc1]

### Step 1: tert-butyl N-[2-[2-[(2-methyl-4-nitro-benzoyl)amino]ethoxy]ethyl]carbamate

The title compound was obtained in analogy to step 1 in the preparation of Reference Example 10 using tert-butyl N-[2-(2-aminoethoxy)ethyl]carbamate and 2-methyl-4-nitro-benzoic acid for condensation. MS (ESI) m/z: 390.1 [M+Na]⁺

### Step 2: tert-butyl N-[2-[2-[(4-amino-2-methyl-benzoyl)amino]ethoxy]ethyl]carbamate

The title compound was obtained in analogy to step 4 in the preparation of Reference Example 450 using tert-butyl N-[2-[2-[(2-methyl-4-nitro-benzoyl)amino]ethoxy]ethyl]carbamate as starting material in hydrogenation. MS (ESI) m/z: 360.2 [M+Na]⁺

### Step 3: 4-(8-chloroimidazo[1,2-a]pyrazin-3-yl)phenol

The title compound was obtained in analogy to step 1 in the preparation of Reference Example 479 using 8-chloro-3-iodo-imidazo[1,2-a]pyrazine and (4-hydroxyphenyl)boronic acid as reaction parterners. MS (ESI) m/z: 245.9 [M+H]⁺

### Step 4: 3-(4-but-2-ynoxyphenyl)-8-chloro-imidazo[1,2-a]pyrazine

A mixture of 4-(8-chloroimidazo[1,2-a]pyrazin-3-yl)phenol (100.0 mg, 0.410 mmol, 1 eq), 1-bromo-2-butyne (81.2 mg, 0.610 mmol, 1.5 eq) and potassium carbonate (112.52 mg, 0.810 mmol, 2 eq) in DMF (3 mL) was stirred at 25° C. for 16 h. The mixture was filtered and the obtained crude product was purified by flash column to afford 78 mg of 3-(4-but-2-ynoxyphenyl)-8-chloro-imidazo[1,2-a]pyrazine as yellow solid. MS (ESI) m/z: 298.0 [M+H]⁺

### Step 5: tert-butyl N-[2-[2-[[4-[[3-(4-but-2-ynoxyphenyl)imidazo[1,2-a]pyrazin-8-yl]amino]-2-methyl-benzoyl]amino]ethoxy]ethyl]carbamate

A mixture of tert-butyl N-[2-[2-[(4-amino-2-methyl-benzoyl)amino]ethoxy]ethyl]carbamate (80.0 mg, 0.240 mmol, 1 eq), 3-(4-but-2-ynoxyphenyl)-8-chloro-imidazo[1,2-a]pyrazine (70.59 mg, 0.240 mmol, 1 eq), cesium carbonate (231.76 mg, 0.710 mmol, 3 eq), tris(dibenzylideneacetone)dipalladium (0) (21.71 mg, 0.020 mmol, 0.100 eq) and 9,9-dimethyl-4,5-bis(diphenylphosphino)xanthene (13.72 mg, 0.020 mmol, 0.100 eq) in 1,4-dioxane (5 mL) was stirred under N₂ at 115° C. under microwave irradiation for 2 h. The mixture was filtered and concentrated to give the crude product, which was purified by prep-TLC (DCM/MeOH/MeCN=10:1:1) to afford 65 mg of the title compound as light yellow solid. MS (ESI) m/z: 599.3. [M+H]⁺

### Step 6: N-[2-(2-aminoethoxy)ethyl]-4-[[3-(4-but-2-ynoxyphenyl)imidazo[1,2-a]pyrazin-8-yl]amino]-2-methyl-benzamide; 2,2,2-trifluoroacetic Acid

A solution of tert-butyl N-[2-[2-[[4-[[3-(4-but-2-ynoxyphenyl)imidazo[1,2-a]pyrazin-8-yl]amino]-2-methyl-benzoyl]amino]ethoxy]ethyl]carbamate (65.0 mg, 0.110 mmol, 1 eq) in DCM (4 mL) was added trifluoroacetic acid (0.5 mL, 6.49 mmol, 59.78 eq) and stirred at 20° C. for 16 h. The mixture was concentrated and the obtained residue was purified by prep-HPLC (TFA) to afford 20.6 mg of the title compound as white solid. MS (ESI) m/z: 499.2. [M+H]+

### Reference Example 480

### N-[2-(2-aminoethoxy)ethyl]-4-[[3-[4-(cyanomethoxy)-3-fluoro-phenyl]imidazo[1,2-a]pyrazin-8-yl]amino]-2-methyl-benzamide; Formic Acid

### Step 1: 4-(8-chloroimidazo[1,2-a]pyrazin-3-yl)-2-fluoro-phenol

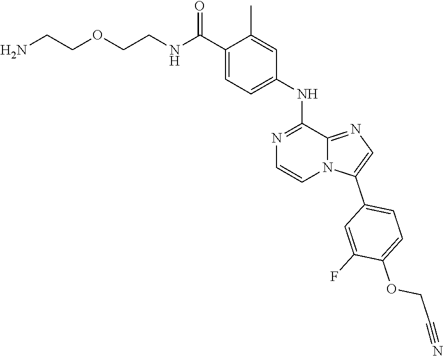 [SMILES: Cc1cc(Nc2nccn3c(-c4ccc(OCC#N)c(F)c4)cnc23)ccc1C(=O)NCCOCCN]

The title compound was obtained in analogy to step 1 in the preparation of Reference Example 479 using 8-chloro-3-iodo-imidazo[1,2-a]pyrazine and 3-fluoro-4-hydroxyphenylboronic acid as starting compounds. MS (ESI) m/z: 264.0 [M+H]⁺

### Step 2: 2-[4-(8-chloroimidazo[1,2-a]pyrazin-3-yl)-2-fluoro-phenoxy]acetonitrile

The title compound was obtained in analogy to step 4 in the preparation of Reference Example 478 using 4-(8-chloroimidazo[1,2-a]pyrazin-3-yl)-2-fluoro-phenol and bromoacetonitrile as reactants. MS (ESI) m/z: 303.0. [M+H]⁺

### Step 3: tert-butyl N-[2-[2-[[4-[[3-[4-(cyanomethoxy)-3-fluoro-phenyl]imidazo[1,2-a]pyrazin-8-yl]amino]-2-methyl-benzoyl]amino]ethoxy]ethyl]carbamate

The title compound was obtained in analogy to step 5 in the preparation of Reference Example 478 using tert-butyl N-[2-[2-[(4-amino-2-methyl-benzoyl)amino]ethoxy]ethyl]carbamate and 2-[4-(8-chloroimidazo[1,2-a]pyrazin-3-yl)-2-fluoro-phenoxy]acetonitrile as coupling reactants. MS (ESI) m/z: 604.5 [M+H]⁺

### Step 4: N-(2-(2-aminoethoxy)ethyl)-4-((3-(4-(cyanomethoxy)-3-fluorophenyl)imidazo[1,2-a]pyrazin-8-yl)amino)-2-methylbenzamide; Formic Acid

The title compound was obtained in analogy to step 6 in the preparation of Reference Example 478 using tert-butyl N-[2-[2-[[4-[[3-[4-(cyanomethoxy)-3-fluoro-phenyl]imidazo[1,2-a]pyrazin-8-yl]amino]-2-methyl-benzoyl]amino]ethoxy]ethyl]carbamate as starting material. MS (ESI) m/z: 504.2 [M+H]⁺

### Reference Example 481

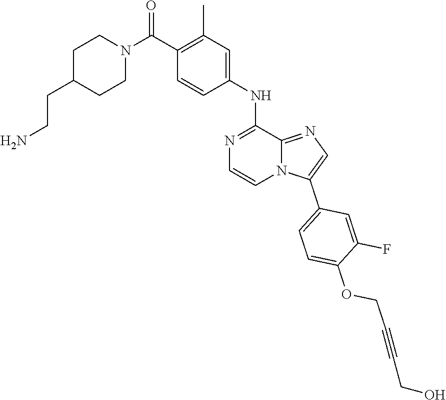 [SMILES: Cc1cc(Nc2nccn3c(-c4ccc(OCC#CCO)c(F)c4)cnc23)ccc1C(=O)N1CCC(CCN)CC1]

### Step 1: 4-hydroxybut-2-yn-1-yl methanesulfonate

The title compound was obtained in analogy to step 4 in the preparation of REF 482 using but-2-yne-1,4-diol as starting material. The product was directly used in crude form.

### Step 2: 4-[4-(8-chloroimidazo[1,2-a]pyrazin-3-yl)-2-fluoro-phenoxy]but-2-yn-1-ol

The title compound was obtained in analogy to step 7 in the preparation of Reference Example 482 using 4-(8-chloroimidazo[1,2-a]pyrazin-3-yl)-2-fluoro-phenol and 4-hydroxybut-2-ynyl methanesulfonate as starting material. MS (ESI) m/z: 332.0 [M+H]⁺

### Step 3: tert-butyl N-[2-[1-(4-amino-2-methyl-benzoyl)-4-piperidyl]ethyl]carbamate

The title compound was obtained in analogy to step 1 in the preparation of Reference Example 482 using 4-amino-2-methyl benzoic acid and 4-(2-BOC-aminoethyl)piperidine for. MS (ESI) m/z: 362.2 [M+H]⁺

### Step 4: tert-butyl N-[2-[1-[4-[[3-[3-fluoro-4-(4-hydroxybut-2-ynoxy)phenyl]imidazo[1,2-a]pyrazin-8-yl]amino]-2-methyl-benzoyl]-4-piperidyl]ethyl]carbamate

The title compound was obtained in analogy to step 2 in the preparation of Reference Example 479 using tert-butyl N-[2-[1-(4-amino-2-methyl-benzoyl)-4-piperidyl]ethyl]carbamate and 4-[4-(8-chloroimidazo[1,2-a]pyrazin-3-yl)-2-fluoro-phenoxy]but-2-yn-1-ol. MS (ESI) m/z: 657.4 [M+H]⁺

### Step 5: [4-(2-aminoethyl)-1-piperidyl]-[4-[[3-[3-fluoro-4-(4-hydroxybut-2-ynoxy)phenyl]imidazo[1,2-a]pyrazin-8-yl]amino]-2-methyl-phenyl]methanone

The title compound was obtained in analogy to step 2 in the preparation of Reference Example 10 using tert-butyl N-[2-[1-[4-[[3-[3-fluoro-4-(4-hydroxybut-2-ynoxy)phenyl]imidazo[1,2-a]pyrazin-8-yl]amino]-2-methyl-benzoyl]-4-piperidyl]ethyl]carbamate as substrate. MS (ESI) m/z: 557.4 [M+H]+

### Reference Example 483

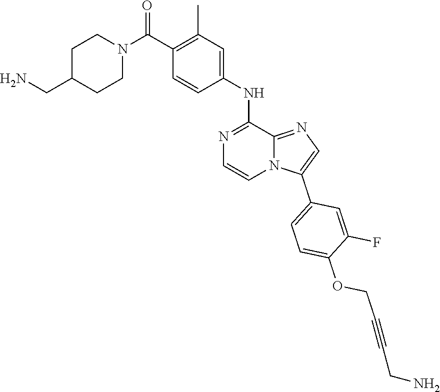 [SMILES: Cc1cc(Nc2nccn3c(-c4ccc(OCC#CCN)c(F)c4)cnc23)ccc1C(=O)N1CCC(CN)CC1]

### [4-[[3-[4-(4-aminobut-2-ynoxy)-3-fluoro-phenyl]imidazo[1,2-a]pyrazin-8-yl]amino]-2-methyl-phenyl]-[4-(aminomethyl)-1-piperidyl]methanone; Formic Acid

### Step 1: 4-((tert-butoxycarbonyl)amino)but-2-yn-1-yl methanesulfonate

The title compound was obtained in analogy to step 4 in the preparation of Reference Example 482 using tert-butyl (4-hydroxybut-2-yn-1-yl)carbamate as starting material. The product was directly used in crude form.

### Step 2: tert-butyl N-[4-[4-(8-chloroimidazo[1,2-a]pyrazin-3-yl)-2-fluoro-phenoxy]but-2-ynyl]carbamate

The title compound was obtained in analogy to step 7 in the preparation of Reference Example 482 using 4-(8-chloroimidazo[1,2-a]pyrazin-3-yl)-2-fluoro-phenol and 4-(tert-butoxycarbonylamino)but-2-ynyl methanesulfonate as starting materials. MS (ESI) m/z: 431.0 [M+H]⁺

### Step 3: tert-butyl ((1-(2-methyl-4-nitrobenzoyl)piperidin-4-yl)methyl)carbamate

The title compound was obtained in analogy to step 1 in the preparation of Reference Example 10 using 4-amino-2-methyl benzoic acid and 4-(tert-butoxycarbonylaminomethyl)piperidine. MS (ESI) m/z: 400.1 [M+Na]⁺

### Step 4: tert-butyl N-[[1-(4-amino-2-methyl-benzoyl)-4-piperidyl]methyl]carbamate

To a stirred suspension mixture of NiCl₂.6H₂O (503.81 mg, 2.12 mmol, 0.500 eq) and sodium borohydride (80.19 mg, 2.12 mmol, 0.500 eq) in methanol (20 mL) was dropwise added a solution of tert-butyl N-[[1-(2-methyl-4-nitro-benzoyl)-4-piperidyl]methyl]carbamate (1.6 g, 4.24 mmol, 1 eq) in THF (6 mL) at 0° C. Then another batch of sodium borohydride (481.14 mg, 12.72 mmol, 3 eq) was added at 0° C. and the reaction mixture was stirred for 1 h at 10° C. The solid was filtered and the solvent was removed in vacuum. The residue was treated with a mixture of EA/H₂O (1/1, 200 mL), the organic layer was washed with sat. NH₄Cl (50 mL) and brine (50 mL), dried over sodium sulfate and filtered, concentrated in vacuum to give tert-butyl N-[[1-(4-amino-2-methyl-benzoyl)-4-piperidyl]methyl]carbamate (1.44 g, 4.14 mmol, 97.77% yield) as white solid. MS (ESI) m/z: 348.1 [M+H]+

### Step 5: tert-butylN-[[1-[4-[[3-[4-[4-(tert-butoxycarbonylamino)but-2-ynoxy]-3-fluoro-phenyl]imidazo[1,2-a]pyrazin-8-yl]amino]-2-methyl-benzoyl]-4-piperidyl]methyl]carbamate

The title compound was obtained in analogy to step 2 in the preparation of Reference Example 479 using tert-butyl N-[[1-(4-amino-2-methyl-benzoyl)-4-piperidyl]methyl]carbamate and tert-butyl N-[4-[4-(8-chloroimidazo[1,2-a]pyrazin-3-yl)-2-fluoro-phenoxy]but-2-ynyl]carbamate. MS (ESI) m/z: 742.5 [M+H]⁺

### Step 6: [4-[[3-[4-(4-aminobut-2-ynoxy)-3-fluoro-phenyl]imidazo[1,2-a]pyrazin-8-yl]amino]-2-methyl-phenyl]-[4-(aminomethyl)-1-piperidyl]methanone; Formic Acid

The title compound was obtained in analogy to step 2 in the preparation of Reference Example 10 using tert-butyl N-[[1-[4-[[3-[4-[4-(tert-butoxycarbonylamino)but-2-ynoxy]-3-fluoro-phenyl]imidazo[1,2-a]pyrazin-8-yl]amino]-2-methyl-benzoyl]-4-piperidyl]methyl]carbamate. MS (ESI) m/z: 542.3 [M+H]⁺

### Reference Example 484

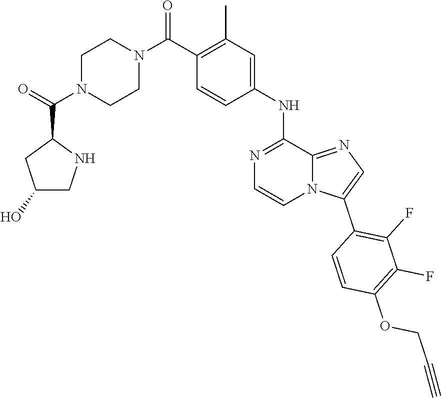 [SMILES: C#CCOc1ccc(-c2cnc3c(Nc4ccc(C(=O)N5CCN(C(=O)[C@@H]6C[C@@H](O)CN6)CC5)c(C)c4)nccn23)c(F)c1F]

### Step 1: tert-butyl 4-(2-methyl-4-nitrobenzoyl)piperazine-1-carboxylate

The title compound was obtained in analogy to step 3 in the preparation of Reference Example 479 using 2-methyl-4-nitrobenzoic acid and tert-butyl piperazine-1-carboxylate. MS (ESI) m/z: 350.2. [M+H]⁺

### Step 2: tert-butyl 4-(4-amino-2-methylbenzoyl)piperazine-1-carboxylate

The title compound was obtained in analogy to step 4 in the preparation of Reference Example 450 using tert-butyl 4-(2-methyl-4-nitrobenzoyl)piperazine-1-carboxylate as substrate. MS (ESI) m/z: 320.2. [M+H]+

### Step 3: tert-butyl 4-[4-[[3-(2,3-difluoro-4-prop-2-ynoxy-phenyl)imidazo[1,2-a]pyrazin-8-yl]amino]-2-methyl-benzoyl]piperazine-1-carboxylate

The title compound was obtained in analogy to step 2 in the preparation of Reference Example 479 using tert-butyl 4-(4-amino-2-methyl-benzoyl)piperazine-1-carboxylate and 8-chloro-3-(2,3-difluoro-4-prop-2-ynoxy-phenyl)imidazo[1,2-a]pyrazine. MS (ESI) m/z: 603.1 [M+H]⁺

### Step 4: [4-[[3-(2,3-difluoro-4-prop-2-ynoxy-phenyl)imidazo[1,2-a]pyrazin-8-yl]amino]-2-methyl-phenyl]-piperazin-1-yl-methanone

The title compound was obtained in analogy to step 2 in the preparation of Reference Example 10 using tert-butyl 4-[4-[[3-(2,3-difluoro-4-prop-2-ynoxy-phenyl)imidazo[1,2-a]pyrazin-8-yl]amino]-2-methyl-benzoyl]piperazine-1-carboxylate as substrate. MS (ESI) m/z: 503.3. [M+H]⁺

### Step 5: tert-butyl (2S,4R)-2-[4-[4-[[3-(2,3-difluoro-4-prop-2-ynoxy-phenyl)imidazo[1,2-a]pyrazin-8-yl]amino]-2-methyl-benzoyl]piperazine-1-carbonyl]-4-hydroxy-pyrrolidine-1-carboxylate

The title compound was obtained in analogy to step 3 in the preparation of Reference Example 479 using [4-[[3-(2,3-difluoro-4-prop-2-ynoxy-phenyl)imidazo[1,2-a]pyrazin-8-yl]amino]-2-methyl-phenyl]-piperazin-1-yl-methanone and (2S,4R)-1-(tert-butoxycarbonyl)-4-hydroxypyrrolidine-2-carboxylic acid. MS (ESI) m/z: 716.3. [M+H]⁺

### Step 6: [4-[[3-(2,3-difluoro-4-prop-2-ynoxy-phenyl)imidazo[1,2-a]pyrazin-8-yl]amino]-2-methyl-phenyl]-[4-[(2S,4R)-4-hydroxypyrrolidine-2-carbonyl]piperazin-1-yl]methanone

The title compound was obtained in analogy to step 2 in the preparation of Reference Example 10 using tert-butyl (2S,4R)-2-[4-[4-[[3-(2,3-difluoro-4-prop-2-ynoxy-phenyl)imidazo[1,2-a]pyrazin-8-yl]amino]-2-methyl-benzoyl]piperazine-1-carbonyl]-4-hydroxy-pyrrolidine-1-carboxylate as substrate. MS (ESI) m/z: 616.3. [M+H]⁺

### Reference Example 485

### [4-(3-aminocyclobutanecarbonyl)piperazin-1-yl]-[4-[[3-[4-(difluoromethoxy)phenyl]imidazo[1,2-a]pyrazin-8-yl]amino]-2-methyl-phenyl]methanone Hydrochloride

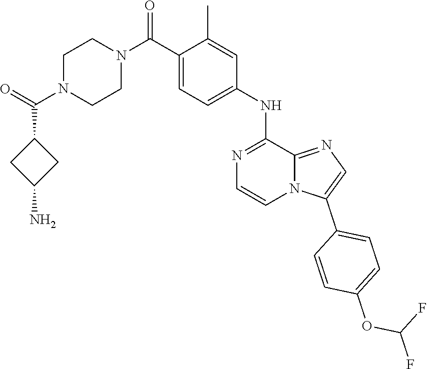 [SMILES: Cc1cc(Nc2nccn3c(-c4ccc(OC(F)F)cc4)cnc23)ccc1C(=O)N1CCN(C(=O)[C@H]2C[C@@H](N)C2)CC1]

The title compound was obtained in analogy to Reference Example 486 using (1S,3S)-3-((tert-butoxycarbonyl)amino)cyclobutanecarboxylic acid instead of (1R,3R)-3-((tert-butoxycarbonyl)amino)cyclobutanecarboxylic acid. MS (ESI, m/z): 576.2 [M+H]⁺

### Reference Example 486

### [4-(3-aminocyclobutanecarbonyl)piperazin-1-yl]-[4-[[3-[4-(difluoromethoxy)phenyl]imidazo[1,2-a]pyrazin-8-yl]amino]-2-methyl-phenyl]methanone Hydrochloride

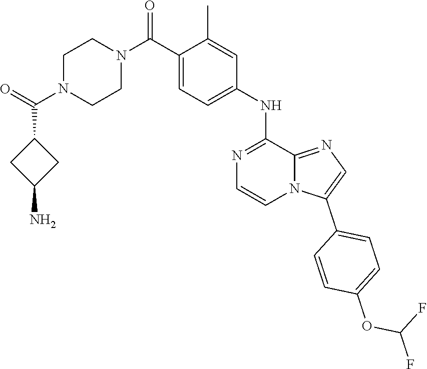 [SMILES: Cc1cc(Nc2nccn3c(-c4ccc(OC(F)F)cc4)cnc23)ccc1C(=O)N1CCN(C(=O)[C@H]2C[C@H](N)C2)CC1]

### Step 1: tert-butyl 4-(4-((3-(4-(difluoromethoxy)phenyl)imidazo[1,2-a]pyrazin-8-yl)amino)-2-methylbenzoyl)piperazine-1-carboxylate

The title compound was obtained in analogy to step 2 in the preparation of Reference Example 479 using 8-chloro-3-[4-(difluoromethoxy)phenyl]imidazo[1,2-a]pyrazine and tert-butyl 4-(4-amino-2-methyl-benzoyl)piperazine-1-carboxylate. MS (ESI) m/z: 579.1 [M+H]⁺

### Step 2: [4-[[3-[4-(difluoromethoxy)phenyl]imidazo[1,2-a]pyrazin-8-yl]amino]-2-methyl-phenyl]-piperazin-1-yl-methanone

The title compound was obtained in analogy to step 2 in the preparation of Reference Example 10 using tert-butyl 4-[4-[[3-[4-(difluoromethoxy)phenyl]imidazo[1,2-a]pyrazin-8-yl]amino]-2-methyl-benzoyl]piperazine-1-carboxylate. MS (ESI) m/z: 479.2 [M+H]⁺

### Step 3: [4-(3-aminocyclobutanecarbonyl)piperazin-1-yl]-[4-[[3-[4-(difluoromethoxy)phenyl]imidazo[1,2-a]pyrazin-8-yl]amino]-2-methyl-phenyl]methanone Hydrochloride

To a mixture of (1r,3r)-3-((tert-butoxycarbonyl)amino)cyclobutanecarboxylic acid (53.98 mg, 0.250 mmol, 1.5 eq) and [4-[[3-[4-(difluoromethoxy)phenyl]imidazo[1,2-a]pyrazin-8-yl]amino]-2-methyl-phenyl]-piperazin-1-yl-methanone (80.0 mg, 0.170 mmol, 1 eq) in DMF (1 mL) was added triethylamine (0.07 mL, 0.500 mmol, 3 eq) and 1-propanephosphonic anhydride (159.59 mg, 0.250 mmol, 1.5 eq) (50% in DMF solution) slowly at 25° C. Then the mixture was stirred at 25° C. for 16 h, and conc. HCl (0.5 mL, 6 mmol, 35.89 eq) was added to the mixture and the mixture was stirred at 25° C. for another 48 h. After that the mixture was filtered and purified by prep-HPLC to give [4-(3-aminocyclobutanecarbonyl)piperazin-1-yl]-[4-[[3-[4-(difluoromethoxy)phenyl]imidazo[1,2-a]pyrazin-8-yl]amino]-2-methyl-phenyl]methanone hydrochloride (31.08 mg, 0.050 mmol, 28.62% yield) as white solid. MS (ESI) m/z: 576.3 [M+H]⁺

### Reference Example 487

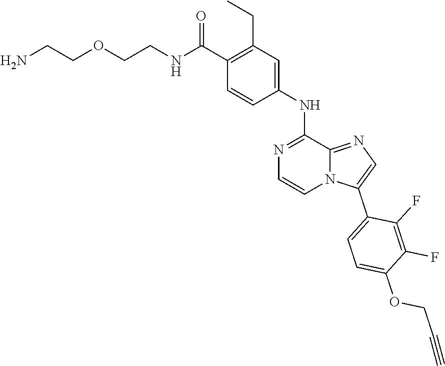 [SMILES: C#CCOc1ccc(-c2cnc3c(Nc4ccc(C(=O)NCCOCCN)c(CC)c4)nccn23)c(F)c1F]

### Step 1: tert-butyl (2-(2-(4-((3-(2,3-difluoro-4-(prop-2-yn-1-yloxy)phenyl)imidazo[1,2-a]pyrazin-8-yl)amino)-2-ethylbenzamido)ethoxy)ethyl)carbamate

The title compound was obtained in analogy to step 3 in the preparation of Reference Example 479 using 4-((3-(2,3-difluoro-4-(prop-2-yn-1-yloxy)phenyl)imidazo[1,2-a]pyrazin-8-yl)amino)-2-ethylbenzoic acid and tert-butyl (2-(2-aminoethoxy)ethyl)carbamate as coupling partners. MS (ESI) m/z: 635.5 [M+H]+

### Step 2: N-(2-(2-aminoethoxy)ethyl)-4-((3-(2,3-difluoro-4-(prop-2-yn-1-yloxy)phenyl)imidazo[1,2-a]pyrazin-8-yl)amino)-2-ethylbenzamide

The title compound was obtained in analogy to step 2 in the preparation of Reference Example 10 using tert-butyl (2-(2-(4-((3-(2,3-difluoro-4-(prop-2-yn-1-yloxy)phenyl)imidazo[1,2-a]pyrazin-8-yl)amino)-2-ethylbenzamido)ethoxy)ethyl)carbamate as substrate. MS (ESI) m/z: 535.3 [M+H]⁺

### Reference Example 488

### 2-[2-[2-[[4-[[3-(2,3-difluoro-4-methoxy-phenyl)imidazo[1,2-a]pyrazin-8-yl]amino]-2-ethyl-benzoyl]amino]ethoxy]ethylamino]acetic Acid; Formic Acid

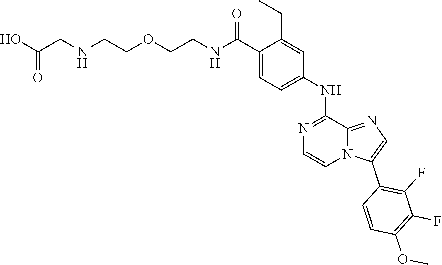 [SMILES: CCc1cc(Nc2nccn3c(-c4ccc(OC)c(F)c4F)cnc23)ccc1C(=O)NCCOCCNCC(=O)O]

### Step 1: 2,5-dioxopyrrolidin-1-yl 4-((3-(2,3-difluoro-4-methoxyphenyl)imidazo[1,2-a]pyrazin-8-yl)amino)-2-ethylbenzoate

To a solution of 4-((3-(2,3-difluoro-4-methoxyphenyl)imidazo[1,2-a]pyrazin-8-yl)amino)-2-ethylbenzoic acid (2.0 g, 4.71 mmol, 1 eq) in THF (30 mL)/DMF (10 mL) was added N-hydroxysuccinimide (813.55 mg, 7.07 mmol, 1.5 eq) and 1-ethyl-(3-(3-dimethylamino)propyl)-carbodiimide hydrochloride (1084.07 mg, 5.66 mmol, 1.2 eq). The mixture was stirred at 25° C. for 3 h. The solvent was evaporated and water was added. The resulting suspension was filtered and the solid was dried in vacuo to give the title compound (1.8 g, 3.45 mmol, 73% yield) as light yellow solid. MS (ESI) m/z: 522 [M+H]⁺

### Step 2: N-(2-(2-aminoethoxy)ethyl)-4-((3-(2,3-difluoro-4-methoxyphenyl)imidazo[1,2-a]pyrazin-8-yl)amino)-2-ethylbenzamide

To a solution of 2,5-dioxopyrrolidin-1-yl 4-((3-(2,3-difluoro-4-methoxyphenyl)imidazo[1,2-a]pyrazin-8-yl)amino)-2-ethylbenzoate (1100 mg, 2.11 mmol, 1 eq) in THF (20 mL) was added triethylamine (0.44 mL, 3.16 mmol, 1.5 eq) and 1,5-diamino-3-oxapentane (329 mg, 3.16 mmol, 1.5 eq). The mixture was stirred at 25° C. for 3 h. The solvent was evaporated. The solid was triturated with water and filtered to afford the title compound (912 mg, 1.79 mmol) as off-white solid. MS (ESI) m/z: 511 [M+H]⁺

### Step 3: methyl 2-((2-(2-(4-((3-(2,3-difluoro-4-methoxyphenyl)imidazo[1,2-a]pyrazin-8-yl)amino)-2-ethylbenzamido)ethoxy)ethyl)amino)acetate

To a solution of N-(2-(2-aminoethoxy)ethyl)-4-((3-(2,3-difluoro-4-methoxyphenyl)imidazo[1,2-a]pyrazin-8-yl)amino)-2-ethylbenzamide (500 mg, 0.980 mmol, 1 eq) in DMF (5 mL) was added triethylamine (0.2 mL, 1.47 mmol, 1.5 eq) and methyl chloroacetate (138 mg, 1.27 mmol, 1.3 eq). The mixture was stirred at 25° C. for 4 h. To the mixture was added water and the pH of mixture was adjusted to around 4 by 1 M aq. HCl. The mixture was extracted with ethyl acetate (50 mL×3). The pH of aqueous solution was adjusted to around 8. The resulting suspension was filtered and the residue was dried to give the title compound (500 mg, 0.980 mmol, 1 eq) as a yellow solid. MS (ESI) m/z: 583 [M+H]⁺

### Step 4: 2-((2-(2-(4-((3-(2,3-difluoro-4-methoxyphenyl)imidazo[1,2-a]pyrazin-8-yl)amino)-2-ethylbenzamido)ethoxy)ethyl)amino)acetic Acid; Formic Acid

To a solution of methyl 2-((2-(2-(4-((3-(2,3-difluoro-4-methoxyphenyl)imidazo[1,2-a]pyrazin-8-yl)amino)-2-ethylbenzamido)ethoxy)ethyl)amino)acetate (50 mg, 0.09 mmol, 1 eq) in methanol (2 mL) was added aq. sodium hydroxide solution (0.5 mL, 2 mmol, 23.3 eq, 4 N). The mixture was stirred at 25° C. for 4 h. The pH was adjusted to around 6 by addition of 2 M aq. HCl. The solvent was evaporated and the residue was purified by prep. HPLC to give the title compound (18 mg, 0.030 mmol) as a white solid. MS (ESI) m/z: 569 [M+H]⁺

### Reference Example 489

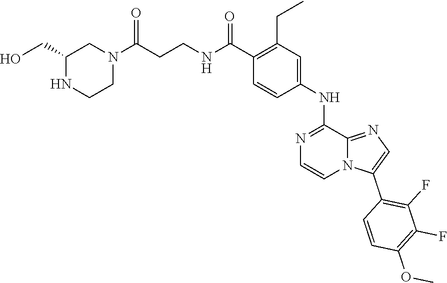 [SMILES: CCc1cc(Nc2nccn3c(-c4ccc(OC)c(F)c4F)cnc23)ccc1C(=O)NCCC(=O)N1CCN[C@H](CO)C1]

### Step 1: 3-[[4-[[3-(2,3-difluoro-4-methoxy-phenyl)imidazo[1,2-a]pyrazin-8-yl]amino]-2-ethyl-benzoyl]amino]propanoic Acid

The title compound was obtained in analogy to step 3 in the preparation of Reference Example 479 using 4-[[3-(2,3-difluoro-4-methoxy-phenyl)imidazo[1,2-a]pyrazin-8-yl]amino]-2-ethyl-benzoic acid and beta-alanine. MS (ESI) m/z: 496.3. [M+H]⁺

### Step 2: tert-butyl (2S)-4-[3-[[4-[[3-(2,3-difluoro-4-methoxy-phenyl)imidazo[1,2-a]pyrazin-8-yl]amino]-2-ethyl-benzoyl]amino]propanoyl]-2-(hydroxymethyl)piperazine-1-carboxylate

The title compound was obtained in analogy to step 3 in the preparation of Reference Example 479 using 3-[[4-[[3-(2,3-difluoro-4-methoxy-phenyl)imidazo[1,2-a]pyrazin-8-yl]amino]-2-ethyl-benzoyl]amino]propanoic acid and tert-butyl (2S)-2-(hydroxymethyl)piperazine-1-carboxylate as reactants. MS (ESI) m/z: 694.2 [M+H]⁺

### Step 3: 4-[[3-(2,3-difluoro-4-methoxy-phenyl)imidazo[1,2-a]pyrazin-8-yl]amino]-2-ethyl-N-[3-[(3S)-3-(hydroxymethyl)piperazin-1-yl]-3-oxo-propyl]benzamide

The title compound was obtained in analogy to step 2 in the preparation of Reference Example 10 using tert-butyl (2S)-4-[3-[[4-[[3-(2,3-difluoro-4-methoxy-phenyl)imidazo[1,2-a]pyrazin-8-yl]amino]-2-ethyl-benzoyl]amino]propanoyl]-2-(hydroxymethyl)piperazine-1-carboxylate as starting material. MS (ESI) m/z: 594.3. [M+H]⁺

### Reference Example 490

### 4-[[3-(2,3-difluoro-4-methoxy-phenyl)imidazo[1,2-a]pyrazin-8-yl]amino]-2-ethyl-N-[4-[(3S)-3-(hydroxymethyl)piperazin-1-yl]-4-oxo-butyl]benzamide

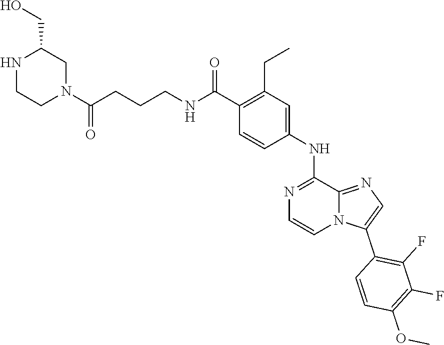 [SMILES: CCc1cc(Nc2nccn3c(-c4ccc(OC)c(F)c4F)cnc23)ccc1C(=O)NCCCC(=O)N1CCN[C@H](CO)C1]

The title compound was obtained in analogy to Reference Example 489 by using 4-aminobutyric acid as starting material instead of beta-alanine. MS (ESI) m/z: 608.4. [M+H]⁺

### Reference Example 491

### 4-[[3-(2,3-difluoro-4-methoxy-phenyl)imidazo[1,2-a]pyrazin-8-yl]amino]-2-ethyl-N-[6-[(3S)-3-(hydroxymethyl)piperazin-1-yl]-6-oxo-hexyl]benzamide

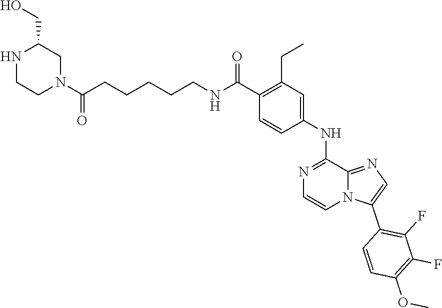 [SMILES: CCc1cc(Nc2nccn3c(-c4ccc(OC)c(F)c4F)cnc23)ccc1C(=O)NCCCCCC(=O)N1CCN[C@H](CO)C1]

The title compound was obtained in analogy to Reference Example 489 by using 6-aminohexanoic acid as starting material instead of beta-alanine. MS (ESI) m/z: 636.4 [M+H]⁺

### Reference Example 492

### 4-[[3-(2,3-difluoro-4-methoxy-phenyl)imidazo[1,2-a]pyrazin-8-yl]amino]-2-ethyl-N-[5-[(3S)-3-(hydroxymethyl)piperazin-1-yl]-5-oxo-pentyl]benzamide

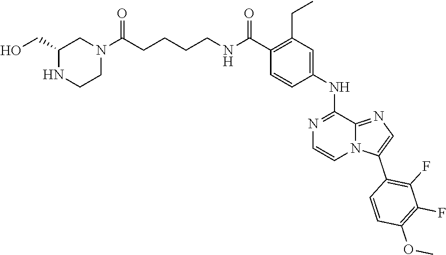 [SMILES: CCc1cc(Nc2nccn3c(-c4ccc(OC)c(F)c4F)cnc23)ccc1C(=O)NCCCCC(=O)N1CCN[C@H](CO)C1]

The title compound was obtained in analogy to Reference Example 489 by using 5-aminovaleric acid as starting material instead of beta-alanine. MS (ESI) m/z: 622.4. [M+H]⁺

### Reference Example 493

### N-[2-(2-amino-1,1-dimethyl-ethoxy)ethyl]-4-[[3-(2,3-difluoro-4-methoxy-phenyl)imidazo[1,2-a]pyrazin-8-yl]amino]-2-ethyl-benzamide; Formic Acid

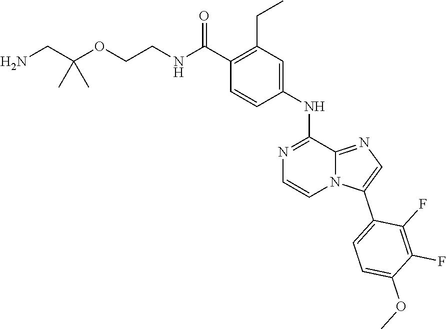 [SMILES: CCc1cc(Nc2nccn3c(-c4ccc(OC)c(F)c4F)cnc23)ccc1C(=O)NCCOC(C)(C)CN]

### Step 1: tert-butyl N-(2-allyloxy-2-methyl-propyl)carbamate

The title compound was obtained in analogy to step 2 in the preparation of Reference Example 494 using tert-butyl 2-hydroxy-2-methylpropylcarbamate and allyl tert-butyl carbonate for coupling reaction.

¹H NMR (400 MHz, CHLOROFORM-d) δ=6.01-5.83 (m, 1H), 5.37-5.10 (m, 2H), 4.86 (br s, 1H), 3.90 (d, J=5.4 Hz, 2H), 3.18 (d, J=5.9 Hz, 2H), 1.46 (s, 9H), 1.20 (s, 6H) ppm.

### Step 2: tert-butyl N-[2-(2-hydroxyethoxy)-2-methyl-propyl]carbamate

The title compound was obtained in analogy to step 3 in the preparation of Reference Example 494 using tert-butyl N-(2-allyloxy-2-methyl-propyl)carbamate for ozonolysis.

¹H NMR (400 MHz, CHLOROFORM-d) δ=4.85 (br s, 1H), 3.64 (t, J=4.3 Hz, 2H), 3.43-3.36 (m, 2H), 3.10 (d, J=6.0 Hz, 2H), 2.10 (br s, 1H), 1.38 (s, 9H), 1.11 (s, 6H) ppm.

### Step 3: 2-[2-(tert-butoxycarbonylamino)-1,1-dimethyl-ethoxy]ethyl Methanesulfonate

The title compound was obtained in analogy to step 4 in the preparation of Reference Example 482 using tert-butyl N-[2-(2-hydroxyethoxy)-2-methyl-propyl]carbamate as starting material. The product was directly used in crude form.

### Step 4: tert-butyl N-[2-(2-azidoethoxy)-2-methyl-propyl]carbamate

The title compound was obtained in analogy to step 5 in the preparation of Reference Example 494 using 2-[2-(tert-butoxycarbonylamino)-1,1-dimethyl-ethoxy]ethyl methanesulfonate and sodium azide.

¹H NMR (400 MHz, CHLOROFORM-d) δ=4.94 (br s, 1H), 3.60-3.53 (m, 1H), 3.60-3.53 (m, 1H), 3.35 (t, J=4.9 Hz, 2H), 3.19 (d, J=6.0 Hz, 2H), 1.47 (s, 9H), 1.21 (s, 6H) ppm.

### Step 5: tert-butyl N-[2-(2-aminoethoxy)-2-methyl-propyl]carbamate

The title compound was obtained in analogy to step 6 in the preparation of Reference Example 494 using tert-butyl N-[2-(2-azidoethoxy)-2-methyl-propyl]carbamate for reduction, used directly in crude form.

### Step 6: tert-butyl N-[2-methyl-2-[2-[(4-nitro-2-vinyl-benzoyl)amino]ethoxy]propyl]carbamate

The title compound was obtained in analogy to step 3 in the preparation of Reference Example 479 using 4-nitro-2-vinyl-benzoic acid and tert-butyl N-[2-(2-aminoethoxy)-2-methyl-propyl]carbamate. MS (ESI) m/z: 420.2 [M+Na]⁺

### Step 7: tert-butyl N-[2-[2-[(4-amino-2-ethyl-benzoyl)amino]ethoxy]-2-methyl-propyl]carbamate

The title compound was obtained in analogy to step 4 of the preparation of Reference Example 495 using tert-butyl N-[2-methyl-2-[2-[(4-nitro-2-vinyl-benzoyl)amino]ethoxy]propyl]carbamate as substrate for this hydrogenation. MS (ESI) m/z: 380.1 [M+H]⁺

### Step 8: tert-butyl N-[2-[2-[[4-[[3-(2,3-difluoro-4-methoxy-phenyl)imidazo[1,2-a]pyrazin-8-yl]amino]-2-ethyl-benzoyl]amino]ethoxy-2-methyl-propyl]carbamate

The title compound was obtained in analogy to step 2 in the preparation of Reference Example 479 using tert-butyl N-[2-[2-[(4-amino-2-ethyl-benzoyl)amino]ethoxy]-2-methyl-propyl]carbamate and 8-chloro-3-(2,3-difluoro-4-methoxy-phenyl)imidazo[1,2-a]pyrazine. MS (ESI) m/z: 639.2 [M+H]⁺

### Step 9: N-[2-(2-amino-1,1-dimethyl-ethoxy)ethyl]-4-[[3-(2,3-difluoro-4-methoxy-phenyl)imidazo[1,2-a]pyrazin-8-yl]amino]-2-ethyl-benzamide

The title compound was obtained in analogy to step 2 in the preparation of Reference Example 10 using tert-butyl N-[2-[2-[[4-[[3-(2,3-difluoro-4-methoxy-phenyl)imidazo[1,2-a]pyrazin-8-yl]amino]-2-ethyl-benzoyl]amino]ethoxy]-2-methyl-propyl]carbamate as substrate. MS (ESI) m/z: 539.3 [M+H]⁺

### Reference Example 482

### 4-[[3-[4-[4-(2-aminoethoxy)but-2-ynoxy]-2,3-difluoro-phenyl]imidazo[1,2-a]pyrazin-8-yl]amino]-N-(3-aminopropyl)-2-ethyl-benzamide

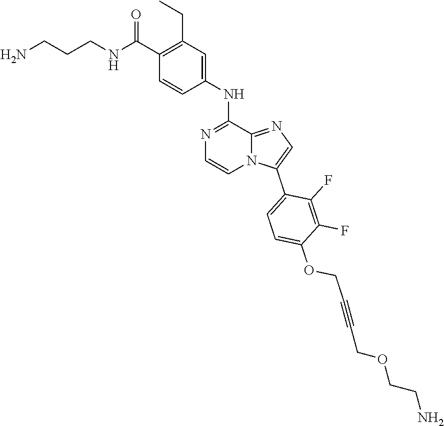 [SMILES: CCc1cc(Nc2nccn3c(-c4ccc(OCC#CCOCCN)c(F)c4F)cnc23)ccc1C(=O)NCCCN]

### Step 1: tert-butyl (3-(2-ethyl-4-nitrobenzamido)propyl)carbamate

The title compound was obtained in analogy to step 3 in the preparation of Reference Example 479 using tert-butyl (3-aminopropyl)carbamate and 2-ethyl-4-nitrobenzoic acid. MS (ESI) m/z: 352.2 [M+H]⁺

### Step 2: tert-butyl (3-(4-amino-2-ethylbenzamido)propyl)carbamate

The title compound was obtained in analogy to step 4 in the preparation of Reference Example 495 using tert-butyl (3-(2-ethyl-4-nitrobenzamido)propyl)carbamate as starting material. MS (ESI) m/z: 332.2 [M+H]⁺

### Step 3: tert-butyl N-[3-[[4-[[3-(2,3-difluoro-4-hydroxy-phenyl)imidazo[1,2-a]pyrazin-8-yl]amino]-2-ethyl-benzoyl]amino]propyl]carbamate

The title compound was obtained in analogy to step 2 in the preparation of Reference Example 479 using 4-(8-chloroimidazo[1,2-a]pyrazin-3-yl)-2,3-difluoro-phenol and tert-butyl N-[3-[(4-amino-2-ethyl-benzoyl)amino]propyl]carbamate as reactants. MS (ESI) m/z: 567.2 [M+H]⁺

### Step 4: 2-(tert-butoxycarbonylamino)ethyl Methanesulfonate

To a solution of N-BOC-ethanolamine (2.0 g, 12.41 mmol, 1 eq) and triethylamine (3.46 mL, 24.81 mmol, 2 eq) in DCM (5 mL) was added methanesulfonyl chloride (1.44 mL, 18.61 mmol, 1.5 eq) at 20° C., the mixture was stirred at 20° C. for 2 h. The mixture was quenched with 1 N aq. HCl, and then the mixture was extracted with ethyl acetate (30 mL×2), and dried over Na₂SO₄, filtered and concentrated to get the title compound (2.5 g, 10.45 mmol, 84.21% yield) as a yellow oil, which was used without further purification.

### Step 5: tert-butyl N-[2-(4-hydroxybut-2-ynoxy)ethyl]carbamate

A mixture of 2-butyne-1,4-diol (1.44 g, 16.72 mmol, 2 eq) and sodium hydroxide in water (8.36 mL, 16.72 mmol, 2 eq) was stirred at 90° C. for 0.5 h, then 2-(tert-butoxycarbonylamino)ethyl methanesulfonate (2.0 g, 8.36 mmol, 1 eq) was added to the mixture at 90° C. and stirred for 5 h. The solution was poured into water and extracted with ethyl acetate (30 mL×2), the combined organic layers were concentrated and the obtained residue was purified by silica gel chromatography (PE/EA=4:1) to afford tert-butyl N-[2-(4-hydroxybut-2-ynoxy)ethyl]carbamate (500 mg, 2.18 mmol) as colorless oil.

### Step 6: 4-[2-(tert-butoxycarbonylamino)ethoxy]but-2-ynyl Methanesulfonate

The title compound was obtained in analogy to step 4 in the preparation of Reference Example 482 using tert-butyl N-[2-(4-hydroxybut-2-ynoxy)ethyl]carbamate as starting material. The product was directly used in crude form.

### Step 7: tert-butyl N-[3-[[4-[[3-[4-[4-[2-(tert-butoxycarbonylamino)ethoxy]but-2-ynoxy]-2,3-difluoro-phenyl]imidazo[1,2-a]pyrazin-8-yl]amino]-2-ethyl-benzoyl]amino]propyl]carbamate

To a solution of tert-butyl N-[3-[[4-[[3-(2,3-difluoro-4-hydroxy-phenyl)imidazo[1,2-a]pyrazin-8-yl]amino]-2-ethyl-benzoyl]amino]propyl]carbamate (50.0 mg, 0.090 mmol, 1 eq) and potassium carbonate (24.39 mg, 0.180 mmol, 2 eq) in acetonitrile (1 mL) was added 4-[2-(tert-butoxycarbonylamino)ethoxy]but-2-ynyl methanesulfonate (40.68 mg, 0.130 mmol, 1.5 eq) at 20° C., the mixture was stirred at 60° C. for 16 h. The mixture was concentrated and purified by prep-HPLC to get the title compound (30 mg, 0.040 mmol, 43.7% yield) as a white solid. MS (ESI) m/z: 778.2 [M+H]⁺

### Step 8: 4-[[3-[4-[4-(2-aminoethoxy)but-2-ynoxy]-2,3-difluoro-phenyl]imidazo[1,2-a]pyrazin-8-yl]amino]-N-(3-aminopropyl)-2-ethyl-benzamide

The title compound was obtained in analogy to step 2 in the preparation of Reference Example 10 using tert-butyl N-[3-[[4-[[3-[4-[4-[2-(tert-butoxycarbonylamino)ethoxy]but-2-ynoxy]-2,3-difluoro-phenyl]imidazo[1,2-a]pyrazin-8-yl]amino]-2-ethyl-benzoyl]amino]propyl]carbamate as reactant. MS (ESI) m/z: 578.4 [M+H]⁺

### Reference Example 494

### N-[2-(2-amino-2-methyl-propoxy)ethyl]-4-[[3-(2,3-difluoro-4-methoxy-phenyl)imidazo[1,2-a]pyrazin-8-yl]amino]-2-ethyl-benzamide; Formic Acid

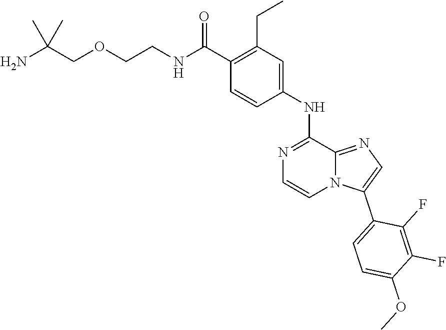 [SMILES: CCc1cc(Nc2nccn3c(-c4ccc(OC)c(F)c4F)cnc23)ccc1C(=O)NCCOCC(C)(C)N]

### Step 1: Allyl Tert-Butyl Carbonate

To a solution of allyl alcohol (19.96 g, 343.64 mmol, 3 eq) and di-tert-butyldicarbonate (25.0 g, 114.55 mmol, 1 eq) was slowly added 4-dimethylaminopyridine (2.8 g, 22.91 mmol, 0.200 eq). The mixture was stirred at 15° C. for 1 h. The mixture was diluted with MTBE, washed with brine, dried over Na₂SO₄ and concentrated. The residue was purified by silica gel chromatography eluting with PE:EA=50:1 to afford allyl tert-butyl carbonate (12 g, 75.86 mmol) as colorless oil.

¹H NMR (400 MHz, CHLOROFORM-d) δ=5.99-5.87 (m, 1H), 5.33 (dd, J=1.4, 17.2 Hz, 1H), 5.24 (dd, J=1.2, 10.4 Hz, 1H), 4.55 (td, J=1.2, 5.8 Hz, 2H), 1.48 (s, 9H) ppm.

### Step 2: tert-butyl N-(2-allyloxy-1,1-dimethyl-ethyl)carbamate

To a mixture of tert-butyl (1-hydroxy-2-methylpropan-2-yl)carbamate (500.0 mg, 2.64 mmol, 1 eq), allyl tert-butyl carbonate (835 mg, 5.28 mmol, 2 eq) in THF (10 mL) was added tetrakis(triphenylphosphine)palladium(0) (610.6 mg, 0.530 mmol, 0.200 eq). The resulting mixture was stirred at 80° C. for 12 h under nitrogen. The mixture was concentrated and the residue was purified by silica gel chromatography eluting with PE:EA=50:1 to afford tert-butyl N-(2-allyloxy-1,1-dimethyl-ethyl)carbamate (250 mg, 1.09 mmol, 41.26% yield) as colorless oil.

¹H NMR (400 MHz, CHLOROFORM-d) δ=5.90-5.75 (m, 1H), 5.26-5.05 (m, 2H), 4.69 (br s, 1H), 3.93 (d, J=5.5 Hz, 2H), 3.30 (s, 2H), 1.36 (s, 9H), 1.23 (s, 6H) ppm.

### Step 3: tert-butyl N-[2-(2-hydroxyethoxy)-1,1-dimethyl-ethyl]carbamate

Through a solution of tert-butyl N-(2-allyloxy-1,1-dimethyl-ethyl)carbamate (250.0 mg, 1.09 mmol, 1 eq) in DCM (20 mL) cooled to −78° C. was bubbled ozone until the mixture turned blue. The mixture was warmed to 0° C. and then sodium borohydride (82.49 mg, 2.18 mmol, 2 eq) was added. The mixture was stirred for 3 h, quenched with saturated NH₄Cl solution and then the organic phase was separated. The mixture was dried over sodium sulfate and concentrated. The residue was purified by silica gel chromatography eluting with PE:EA from 10:1 to 3:1 to afford tert-butyl N-[2-(2-hydroxyethoxy)-1,1-dimethyl-ethyl]carbamate (80 mg, 0.340 mmol, 31.45% yield) as colorless oil.

¹H NMR (400 MHz, CHLOROFORM-d) δ=4.61 (br s, 1H), 3.69-3.65 (m, 2H), 3.55-3.49 (m, 2H), 3.41 (s, 2H), 1.37 (s, 9H), 1.22 (s, 6H) ppm.

### Step 4: 2-[2-(tert-butoxycarbonylamino)-2-methyl-propoxy]ethyl methanesulfonate

The title compound was obtained in analogy to step 4 in the preparation of Reference Example 482 using tert-butyl N-[2-(2-hydroxyethoxy)-1,1-dimethyl-ethyl]carbamate as starting material. The product was directly used in crude form.

### Step 5: tert-butyl N-[2-(2-azidoethoxy)-1,1-dimethyl-ethyl]carbamate

To a solution of 2-[2-(tert-butoxycarbonylamino)-2-methyl-propoxy]ethyl methanesulfonate (550.0 mg, 1.77 mmol, 1 eq) in DMF (5 mL) was added sodium azide (0.31 mL, 8.83 mmol, 5 eq). The resulting mixture was stirred at 50° C. for 2 h. The mixture was diluted with water, extracted with ethyl acetate, washed with brine, dried over sodium sulfate and concentrated. The residue was purified by silica gel chromatography eluting with PE:EA=10:1 to afford tert-butyl N-[2-(2-azidoethoxy)-1,1-dimethyl-ethyl]carbamate (380 mg, 1.47 mmol, 83.29% yield) as colorless oil.

¹H NMR (400 MHz, chloroform-d) 6=4.63 (br s, 1H), 3.62-3.57 (m, 2H), 3.40 (s, 2H), 3.29 (t, J=4.9 Hz, 2H), 1.36 (s, 9H), 1.23 (s, 6H) ppm.

### Step 6: tert-butyl N-[2-(2-aminoethoxy)-1,1-dimethyl-ethyl]carbamate

To a solution of tert-butyl N-[2-(2-azidoethoxy)-1,1-dimethyl-ethyl]carbamate (380.0 mg, 1.47 mmol, 1 eq) in ethyl acetate (5 mL) was added palladium on carbon (38.0 mg, 0.040 mmol, 0.020 eq). The resulting mixture was hydrogenated at 760 mmHg at 15° C. for 2 hand the catalyst was removed by filtration. The filtrate was concentrated to afford tert-butyl N-[2-(2-aminoethoxy)-1,1-dimethyl-ethyl]carbamate (360 mg, 1.55 mmol, crude) as colorless oil, which was used without further purification.

### Step 7: tert-butyl N-[1,1-dimethyl-2-[2-[(4-nitro-2-vinyl-benzoyl)amino]ethoxy]ethyl]carbamate

The title compound was obtained in analogy to step 3 in the preparation of Reference Example 479 using 4-nitro-2-vinyl-benzoic acid and tert-butyl N-[2-(2-aminoethoxy)-1,1-dimethyl-ethyl]carbamate for condensation. MS (ESI) m/z: 430.3. [M+Na]⁺

### Step 8: tert-butyl N-[2-[2-[(4-amino-2-ethyl-benzoyl)amino]ethoxy]-1,1-dimethyl-ethyl]carbamate

The title compound was obtained in analogy to step 4 in the preparation of Reference Example 450 using tert-butyl N-[1,1-dimethyl-2-[2-[(4-nitro-2-vinyl-benzoyl)amino]ethoxy]ethyl]carbamate as substrate. MS (ESI) m/z: 402.3. [M+Na]⁺

### Step 9: tert-butyl N-[2-[2-[[4-[[3-(2,3-difluoro-4-methoxy-phenyl)imidazo[1,2-a]pyrazin-8-yl]amino]-2-ethyl-benzoyl]amino]ethoxy]-1,1-dimethyl-ethyl]carbamate

The title compound was obtained in analogy to step 2 in the preparation of Reference Example 479 using tert-butyl N-[2-[2-[(4-amino-2-ethyl-benzoyl)amino]ethoxy]-1,1-dimethyl-ethyl]carbamate and 8-chloro-3-(2,3-difluoro-4-methoxy-phenyl)imidazo[1,2-a]pyrazine. MS (ESI) m/z: 639.4. [M+H]⁺

### Step 10: N-[2-(2-amino-2-methyl-propoxy)ethyl]-4-[[3-(2,3-difluoro-4-methoxy-phenyl)imidazo[1,2-a]pyrazin-8-yl]amino]-2-ethyl-benzamide; Formic Acid

The title compound was obtained in analogy to step 2 in the preparation of Reference Example 10 using tert-butyl N-[2-[2-[[4-[[3-(2,3-difluoro-4-methoxy-phenyl)imidazo[1,2-a]pyrazin-8-yl]amino]-2-ethyl-benzoyl]amino]ethoxy]-1,1-dimethyl-ethyl]carbamate as substrate. MS (ESI) m/z: 539.2 [M+H]⁺

### Reference Example 496

### 4-[[3-(2,3-difluoro-4-prop-2-ynoxy-phenyl)imidazo[1,2-a]pyrazin-8-yl]amino]-2-ethyl-N-[3-(2-hydroxyethylamino)propyl]benzamide

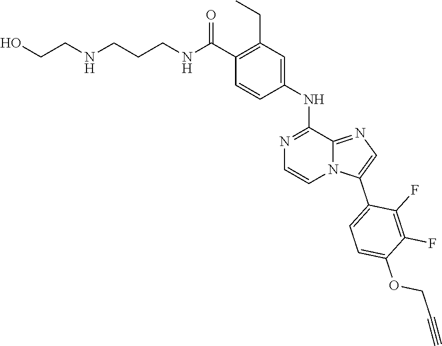 [SMILES: C#CCOc1ccc(-c2cnc3c(Nc4ccc(C(=O)NCCCNCCO)c(CC)c4)nccn23)c(F)c1F]

### Step 1: 4-(8-chloroimidazo[1,2-a]pyrazin-3-yl)-2,3-difluoro-phenol

The title compound was obtained in analogy to step 5 in the preparation of Intermediate 27 using 8-chloro-3-iodo-imidazo[1,2-a]pyrazine and 2,3-difluoro-4-(4,4,5,5-tetramethyl-1,3,2-dioxaborolan-2-yl)phenol. MS (ESI) m/z: 282.0 [M+H]⁺

### Step 2: 8-chloro-3-(2,3-difluoro-4-prop-2-ynoxy-phenyl)imidazo[1,2-a]pyrazine

The title compound was obtained in analogy to step 4 in the preparation of Reference Example 478 using 4-(8-chloroimidazo[1,2-a]pyrazin-3-yl)-2,3-difluoro-phenol and propargyl bromide as reactants. MS (ESI) m/z: 320.1. [M+H]⁺

### Step 3: tert-butyl N-[3-(benzyloxycarbonylamino)propyl]-N-(2-hydroxyethyl)carbamate

To a solution of benzyl N-[3-(2-hydroxyethylamino)propyl]carbamate (120.0 mg, 0.480 mmol, 1 eq) and triethylamine (0.07 mL, 0.480 mmol, 1 eq) in DCM (5 mL) was added di-t-butyldicarbonate (103.8 mg, 0.480 mmol, 1 eq), the mixture was stirred at 20° C. for 16 h. The mixture was concentrated and the obtained residue purified by reverse phase flash column chromatography to get the product tert-butyl N-[3-(benzyloxycarbonylamino)propyl]-N-(2-hydroxyethyl)carbamate (80 mg, 0.230 mmol, 47.73% yield) as a colorless oil. MS (ESI) m/z: 353.2 [M+H]⁺

### Step 4: tert-butyl N-(3-aminopropyl)-N-(2-hydroxyethyl)carbamate

To a solution of tert-butyl N-[3-(benzyloxycarbonylamino)propyl]-N-(2-hydroxyethyl)carbamate (80.0 mg, 0.230 mmol, 1 eq) in methanol (2 mL) was added Pd/C (0.230 mmol, 1 eq) at 20° C., the mixture was stirred at 20° C. for 24 h, filtered and concentrated to get the product tert-butyl N-(3-aminopropyl)-N-(2-hydroxyethyl)carbamate (30 mg, 0.140 mmol, 40.36% yield) as a colorless oil, which was used without further purification in the next step.

### Step 5: 4-((3-(2,3-difluoro-4-(prop-2-yn-1-yloxy)phenyl)imidazo[1,2-a]pyrazin-8-yl)amino)-2-ethylbenzoic Acid

The title compound was obtained in analogy to step 2 in the preparation of Reference Example 479 using 8-chloro-3-(2,3-difluoro-4-prop-2-ynoxy-phenyl)imidazo[1,2-a]pyrazine and 4-amino-2-ethylbenzoic acid as reactants. MS (ESI) m/z: 449.1 [M+H]⁺

### Step 6: tert-butyl N-[3-[[4-[[3-(2,3-difluoro-4-prop-2-ynoxy-phenyl)imidazo[1,2-a]pyrazin-8-yl]amino]-2-ethyl-benzoyl]amino]propyl]-N-(2-hydroxyethyl)carbamate

The title compound was obtained in analogy to step 3 in the preparation of Reference Example 479 using 4-((3-(2,3-difluoro-4-(prop-2-yn-1-yloxy)phenyl)imidazo[1,2-a]pyrazin-8-yl)amino)-2-ethylbenzoic acid and tert-butyl N-(3-aminopropyl)-N-(2-hydroxyethyl)carbamate as coupling partners. MS (ESI) m/z: 649.3 [M+H]⁺

### Step 7: 4-[[3-(2,3-difluoro-4-prop-2-ynoxy-phenyl)imidazo[1,2-a]pyrazin-8-yl]amino]-2-ethyl-N-[3-(2-hydroxyethylamino)propyl]benzamide

The title compound was obtained in analogy to step 2 in the preparation of Reference Example 10 using tert-butyl N-[3-[[4-[[3-(2,3-difluoro-4-prop-2-ynoxy-phenyl)imidazo[1,2-a]pyrazin-8-yl]amino]-2-ethyl-benzoyl]amino]propyl]-N-(2-hydroxyethyl)carbamate as starting material. MS (ESI) m/z: 549.4 [M+H]⁺

### Reference Example 479

### 4-((3-(2,3-difluoro-4-methoxyphenyl)imidazo[1,2-a]pyrazin-8-yl)amino)-2-ethyl-N-(2-(methyl(2-(methylamino)ethyl)amino)-2-oxoethyl)benzamide; Formic Acid

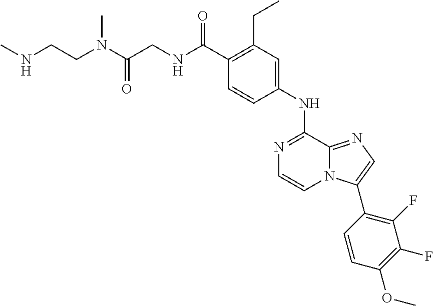 [SMILES: CCc1cc(Nc2nccn3c(-c4ccc(OC)c(F)c4F)cnc23)ccc1C(=O)NCC(=O)N(C)CCNC]

### Step 1: 8-chloro-3-(2,3-difluoro-4-methoxyphenyl)imidazo[1,2-a]pyrazine

A mixture of 8-chloro-3-iodo-imidazo[1,2-a]pyrazine (10.0 g, 35.78 mmol, 1 eq), 2,3-difluoro-4-methoxyphenylboronic acid (8.07 g, 42.94 mmol, 1.2 eq), [1,1′-bis(diphenylphosphino)ferrocene]dichloropalladium(II) (2.62 g, 3.58 mmol, 0.100 eq) and sodium carbonate (7.58 g, 71.56 mmol, 2 eq) in 1,4-dioxane (72 mL) and water (8 mL) was stirred for 15 h at 80° C. under N₂. The mixture was filtered and the filtrate was concentrated in vacuo to give a crude product, which was purified by silica gel chromatography eluting with petroleum ether/ethyl acetate=2:1 to give product 8-chloro-3-(2,3-difluoro-4-methoxy-phenyl)imidazo[1,2-a]pyrazine (6 g, 20.29 mmol, 50.81% yield) as a light yellow solid. MS (ESI) m/z: 296.0 [M+H]⁺

### Step 2: 4-((3-(2,3-difluoro-4-methoxyphenyl)imidazo[1,2-a]pyrazin-8-yl)amino)-2-ethylbenzoic Acid

A mixture of 4-amino-2-ethyl-benzoic acid (216.91 mg, 1.12 mmol, 1.1 eq) and 8-chloro-3-(2,3-difluoro-4-methoxy-phenyl)imidazo[1,2-a]pyrazine (300.0 mg, 1.01 mmol, 1 eq) in acetonitrile (6.3 mL) and acetic acid (0.700 mL) was stirred for 12 h at 65° C. The solvent was removed in vacuo to give 4-[[3-(2,3-difluoro-4-methoxy-phenyl)imidazo[1,2-a]pyrazin-8-yl]amino]-2-ethyl-benzoic acid (400 mg, 0.940 mmol, 72.64% yield) as an off-white solid, which was used without further purification in the next step. MS (ESI) m/z: 425.0 [M+H]⁺

### Step 3: tert-butyl 2-(4-((3-(2,3-difluoro-4-methoxyphenyl)imidazo[1,2-a]pyrazin-8-yl)amino)-2-ethylbenzamido)acetate

To a solution of 4-[[3-(2,3-difluoro-4-methoxy-phenyl)imidazo[1,2-a]pyrazin-8-yl]amino]-2-ethyl-benzoic acid (400.0 mg, 0.940 mmol, 1 eq) in DMF (8 mL) was added 0-(7-azabenzotriazol-1-yl)-N,N,N′,N′-tetramethyluronium hexafluorophosphate (430 mg, 1.13 mmol, 1.2 eq) and N,N-diisopropylethylamine (0.33 mL, 1.89 mmol, 2 eq), then the mixture was stirred for 0.2 h at 10° C., tert-butyl glycinate (135.99 mg, 1.04 mmol, 1.1 eq) was added and the mixture was stirred for 15 h at 10° C. The mixture was diluted with water, filtered and dried to give tert-butyl 2-[[4-[[3-(2,3-difluoro-4-methoxy-phenyl)imidazo[1,2-a]pyrazin-8-yl]amino]-2-ethyl-benzoyl]amino]acetate (536 mg, 1 mmol, 88% yield) as a light yellow solid. MS (ESI) m/z: 538.1 [M+H]⁺

### Step 4: 2-(4-((3-(2,3-difluoro-4-methoxyphenyl)imidazo[1,2-a]pyrazin-8-yl)amino)-2-ethylbenzamido)acetic Acid

To a solution of tert-butyl 2-[[4-[[3-(2,3-difluoro-4-methoxy-phenyl)imidazo[1,2-a]pyrazin-8-yl]amino]-2-ethyl-benzoyl]amino]acetate (536.0 mg, 0.830 mmol, 1 eq) in 1,4-dioxane (6 mL) was added 4 M HCl in dioxane (6.22 mL, 24.89 mmol, 30 eq), and the mixture was stirred for 15 h at 30° C. The solvent was evaporated to give crude 2-[[4-[[3-(2,3-difluoro-4-methoxy-phenyl)imidazo[1,2-a]pyrazin-8-yl]amino]-2-ethyl-benzoyl]amino]acetic acid (455 mg, 0.950 mmol, 91.14% yield) as an off-white solid, which was used in the next step without further purification. MS (ESI) m/z: 482.2 [M+H]⁺

### Step 5: tert-butyl (2-(2-(4-((3-(2,3-difluoro-4-methoxyphenyl)imidazo[1,2-a]pyrazin-8-yl)amino)-2-ethylbenzamido)-N-methylacetamido)ethyl)(methyl)carbamate

The title compound was obtained in analogy to step 1 in the preparation of Reference Example 10 using 2-(4-((3-(2,3-difluoro-4-methoxyphenyl)imidazo[1,2-a]pyrazin-8-yl)amino)-2-ethylbenzamido)acetic acid and tert-butyl N-methyl-N-[2-(methylamino)ethyl]carbamate as reactants. MS (ESI) m/z: 652.3 [M+H]⁺

### Step 6: 4-((3-(2,3-difluoro-4-methoxyphenyl)imidazo[1,2-a]pyrazin-8-yl)amino)-2-ethyl-N-(2-(methyl(2-(methylamino)ethyl)amino)-2-oxoethyl)benzamide

The title compound was obtained in analogy to step 2 in the preparation of Reference Example 10 using tert-butyl (2-(2-(4-((3-(2,3-difluoro-4-methoxyphenyl)imidazo[1,2-a]pyrazin-8-yl)amino)-2-ethylbenzamido)-N-methylacetamido)ethyl)(methyl)carbamate as reactant. MS (ESI) m/z: 552.1 [M+H]⁺

### Reference Example 497

### N-(3-aminopropyl)-4-[[3-(2,3-difluoro-4-methoxy-phenyl)imidazo[1,2-a]pyrazin-8-yl]amino]-2-(difluoromethyl)benzamide; Formic Acid

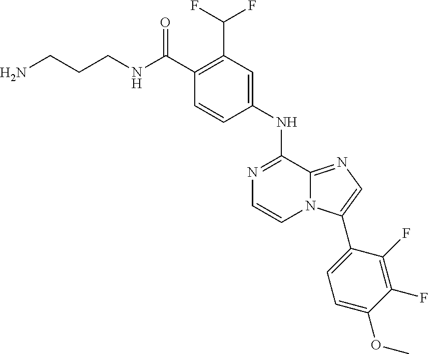 [SMILES: COc1ccc(-c2cnc3c(Nc4ccc(C(=O)NCCCN)c(C(F)F)c4)nccn23)c(F)c1F]

### Step 1: methyl 2-(difluoromethyl)-4-nitrobenzoate

A solution of methyl 2-formyl-4-nitro-benzoate (500.0 mg, 2.39 mmol, 1 eq) in DCM (20 mL) was cooled to −15° C. and diethylaminosulfur trifluoride (1926.64 mg, 11.95 mmol, 5 eq) was added. The resulting mixture was stirred at 10° C. for 15 h. The mixture was quenched with sat. NaHCO₃. The organic separated layer was dried over sodium sulfate and concentrated. The residue was purified by silica gel chromatography eluting with PE:EA=100:1 to afford methyl 2-(difluoromethyl)-4-nitro-benzoate (380 mg, 1.64 mmol, 68.77% yield) as yellow oil.

¹H NMR (400 MHz, CHLOROFORM-d) δ=8.68 (d, J=2.0 Hz, 1H), 8.41 (dd, J=2.3, 8.5 Hz, 1H), 8.24 (d, J=8.5 Hz, 1H), 7.70-7.41 (m, 1H), 4.03 (s, 3H) ppm.

### Step 2: 2-(difluoromethyl)-4-nitrobenzoic Acid

To a solution of methyl 2-(difluoromethyl)-4-nitro-benzoate (380.0 mg, 1.64 mmol, 1 eq) in THF (10 mL) and water (1 mL) was added LiOH.H₂O (137.7 mg, 3.28 mmol, 2 eq). The resulting mixture was stirred at 10° C. for 2 h. The mixture was acidified with 1N HCl to pH=3 and extracted with ethyl acetate (50 mL×2), washed with brine, dried over Na₂SO₄ and concentrated to afford 2-(difluoromethyl)-4-nitro-benzoic acid (350 mg, 1.61 mmol, 98.05% yield) as yellow solid, which was used directly in next step.

### Step 3: tert-butyl N-[3-[[2-(difluoromethyl)-4-nitro-benzoyl]amino]propyl]carbamate

The title compound was obtained in analogy to step 3 in the preparation of Reference Example 479 using 2-(difluoromethyl)-4-nitro-benzoic acid and N-BOC-1,3-diaminopropane. MS (ESI) m/z: 396.3. [M+Na]⁺

### Step 4: tert-butyl N-[3-[[4-amino-2-(difluoromethyl)benzoyl]amino]propyl]carbamate

The title compound was obtained in analogy to step 4 in the preparation of Reference Example 450 using tert-butyl N-[3-[[2-(difluoromethyl)-4-nitro-benzoyl]amino]propyl] carbamate as substrate for hydrogenation. MS (ESI) m/z: 366.1 [M+Na]⁺

### Step 5: tert-butyl N-[3-[[4-[[3-(2,3-difluoro-4-methoxy-phenyl)imidazo[1,2-a]pyrazin-8-yl]amino]-2-(difluoromethyl)benzoyl]amino]propyl]carbamate

A mixture of 8-chloro-3-(2,3-difluoro-4-methoxy-phenyl)imidazo[1,2-a]pyrazine (70.0 mg, 0.240 mmol, 1 eq), tert-butyl N-[3-[[4-amino-2-(difluoromethyl)benzoyl]amino]propyl]carbamate (97.55 mg, 0.280 mmol, 1.2 eq), Brettphos Pd G3 (21.46 mg, 0.020 mmol, 0.100 eq), potassium carbonate (98.16 mg, 0.710 mmol, 3 eq) in tert-butanol (5 mL) were stirred for 15 h at 110° C. under nitrogen protection. The mixture was filtered and the solvent was removed in vacuum to give crude product, which was purified by prep-TLC (DCM/MeOH=10/1) to give product tert-butyl N-[3-[[4-[[3-(2,3-difluoro-4-methoxy-phenyl)imidazo[1,2-a]pyrazin-8-yl]amino]-2-(difluoromethyl)benzoyl]amino]propyl]carbamate (110 mg, 0.180 mmol, 77% yield). MS (ESI) m/z: 603.2 [M+H]⁺

### Step 6: N-(3-aminopropyl)-4-[[3-(2,3-difluoro-4-methoxy-phenyl)imidazo[1,2-a]pyrazin-8-yl]amino]-2-(difluoromethyl)benzamide

The title compound was obtained in analogy to step 2 in the preparation of Reference Example 10 using tert-butyl N-[3-[[4-[[3-(2,3-difluoro-4-methoxy-phenyl)imidazo[1,2-a]pyrazin-8-yl]amino]-2-(difluoromethyl)benzoyl]amino]propyl]carbamate as substrate. MS (ESI) m/z: 503.3 [M+H]⁺

### Reference Example 498

### 4-[[3-[4-(difluoromethoxy)phenyl]imidazo[1,2-a]pyrazin-8-yl]amino]-2-isopropyl-N-methyl-benzamide

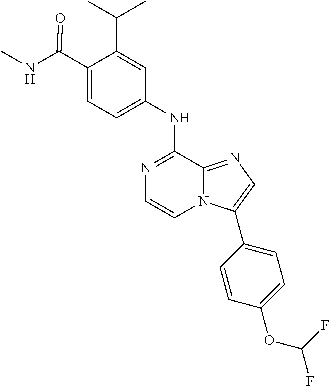 [SMILES: CNC(=O)c1ccc(Nc2nccn3c(-c4ccc(OC(F)F)cc4)cnc23)cc1C(C)C]

### Step 1: methyl 2-isopropenyl-4-nitro-benzoate

The title compound was obtained in analogy to step 1 in the preparation of Reference Example 499 using 2-bromo-4-nitro-benzoate and 4,4,5,5-tetramethyl-2-(prop-1-en-2-yl)-1,3,2-dioxaborolane.

¹H NMR (400 MHz, chloroform-d) δ=8.20-8.12 (m, 2H), 7.94-7.90 (m, 1H), 5.25 (quin, J=1.4 Hz, 1H), 4.99-4.94 (m, 1H), 3.92 (s, 3H), 2.14 (dd, J=0.9, 1.5 Hz, 3H) ppm.

### Step 2: 2-isopropenyl-N-methyl-4-nitro-benzamide

The title compound was obtained in analogy to step 4 in the preparation of Reference Example 450 using methyl 2-isopropenyl-4-nitro-benzoate.

### Step 3: 4-amino-2-isopropyl-N-methyl-benzamide

The title compound was obtained in analogy to step 3 in the preparation of Reference Example 499 using 2-isopropenyl-N-methyl-4-nitro-benzamide as substrate. MS (ESI) m/z: 193.2 [M+H]⁺

### Step 4: 4-[[3-[4-(difluoromethoxy)phenyl]imidazo[1,2-a]pyrazin-8-yl]amino]-2-isopropyl-N-methyl-benzamide

The title compound was obtained in analogy to step 2 in the preparation of Reference Example 479 using 8-chloro-3-(4-(difluoromethoxy)phenyl)imidazo[1,2-a]pyrazine and 4-amino-2-isopropyl-N-methyl-benzamide for this substitution reaction. MS (ESI) m/z: 452.2 [M+H]⁺

### Reference Example 500

### N-[2-(2-aminoethoxy)ethyl]-4-[[3-(2,3-difluoro-4-methoxy-phenyl)imidazo[1,2-a]pyrazin-8-yl]amino]-2-(2-fluoroethyl)benzamide

### Step 1: tert-butyl 2-formyl-4-nitrobenzoate

To a solution of tert-butyl 4-nitro-2-vinyl-benzoate (3.2 g, 12.84 mmol, 1 eq) in DCM (50 mL) cooled to −78 was bubbled ozone (6162.24 mg, 128.38 mmol, 10 eq) until the reaction mixture turn blue, and the nitrogen was bubbled for 5 min. Dimethylsulfide (10.0 mL, 12.84 mmol, 1 eq) was added, the resulting mixture was stirred at 25° C. for 15 h. The mixture was concentrated and the residue was purified by silica gel chromatography eluting with PE:EA=10:1 to afford tert-butyl 2-formyl-4-nitro-benzoate (1.9 g, 7.56 mmol, 58.91% yield) as white solid. MS (ESI) m/z: 252.1 [M+H]⁺

### Step 2: tert-butyl 2-[(E)-2-bromo-2-fluoro-vinyl]-4-nitro-benzoate

To a solution of tert-butyl 2-formyl-4-nitro-benzoate (0.6 g, 2.39 mmol, 1 eq) and triphenylphosphine (2505.51 mg, 9.55 mmol, 4 eq) in THF (20 mL) was added tribromo(fluoro)methane (1616.3 mg, 5.97 mmol, 2.5 eq). The resulting mixture was stirred at 70° C. under nitrogen for 15 h. The mixture was concentrated and then purified by silica gel chromatography eluting with PE:EA=20:1 to afford tert-butyl 2-[(E)-2-bromo-2-fluoro-vinyl]-4-nitro-benzoate (600 mg, 1.73 mmol, 73% yield) as white solid.

### Step 3: tert-butyl 4-amino-2-(2-fluoroethyl)benzoate

The title compound was obtained in analogy to step 4 in the preparation of Reference Example 450 using tert-butyl 2-[(E)-2-bromo-2-fluoro-vinyl]-4-nitro-benzoate. MS (ESI) m/z: 240.1 [M+H]⁺

### Step 4: 4-[[3-(2,3-difluoro-4-methoxy-phenyl)imidazo[1,2-a]pyrazin-8-yl]amino]-2-(2-fluoroethyl)benzoic Acid

A solution of tert-butyl 4-amino-2-(2-fluoroethyl)benzoate (194.24 mg, 0.810 mmol, 1.2 eq) and 8-chloro-3-(2,3-difluoro-4-methoxy-phenyl)imidazo[1,2-a]pyrazine (200.0 mg, 0.680 mmol, 1 eq) in ACN (4.5 mL) and acetic acid (0.500 mL) was heated to 80° C. for 15 h. The mixture was purified by prep-HPLC to afford 4-[[3-(2,3-difluoro-4-methoxy-phenyl)imidazo[1,2-a]pyrazin-8-yl]amino]-2-(2-fluoroethyl)benzoic acid (160 mg, 0.360 mmol, 53% yield) as off-white solid. MS (ESI) m/z: 443.2 [M+H]⁺

### Step 5: tert-butyl N-[2-[2-[[4-[[3-(2,3-difluoro-4-methoxy-phenyl)imidazo[1,2-a]pyrazin-8-yl]amino]-2-(2-fluoroethyl)benzoyl]amino]ethoxy]ethyl]carbamate

The title compound was obtained in analogy to step 3 in the preparation of Reference Example 479 using 4-[[3-(2,3-difluoro-4-methoxy-phenyl)imidazo[1,2-a]pyrazin-8-yl]amino]-2-(2-fluoroethyl)benzoic acid and tert-butyl (2-(2-aminoethoxy)ethyl)carbamate. MS (ESI) m/z: 629.1 [M+H]⁺

### Step 6: N-[2-(2-aminoethoxy)ethyl]-4-[[3-(2,3-difluoro-4-methoxy-phenyl)imidazo[1,2-a]pyrazin-8-yl]amino]-2-(2-fluoroethyl)benzamide

The title compound was obtained in analogy to step 2 in the preparation of Reference Example 10 using tert-butyl N-[2-[2-[[4-[[3-(2,3-difluoro-4-methoxy-phenyl)imidazo[1,2-a]pyrazin-8-yl]amino]-2-(2-fluoroethyl)benzoyl]amino]ethoxy]ethyl]carbamate as. MS (ESI) m/z: 529.3 [M+H]⁺

### Reference Example 499

### 2-cyclopropyl-4-[[3-[4-(difluoromethoxy)phenyl]imidazo[1,2-a]pyrazin-8-yl]amino]-N-methyl-benzamide

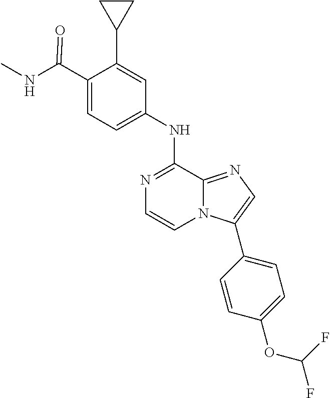 [SMILES: CNC(=O)c1ccc(Nc2nccn3c(-c4ccc(OC(F)F)cc4)cnc23)cc1C1CC1]

### Step 1: 2-cyclopropyl-4-nitro-benzoate

A solution of methyl 2-bromo-4-nitro-benzoate (600.0 mg, 2.31 mmol, 1 eq), cyclopropylboronic acid (594.49 mg, 6.92 mmol, 3 eq), potassium phosphate (0.96 mL, 11.54 mmol, 5 eq) and 1,1′-bis(di-tert-butylphosphino)ferrocene palladium dichloride (150 mg, 0.230 mmol, 0.100 eq) in toluene (10 mL) and water (0.4 mL) was heated to 100° C. for 15 h under nitrogen. The reaction mixture was concentrated and the residue was purified by silica gel chromatography eluting with PE:EA from 10:1 to 5:1 to afford methyl 2-cyclopropyl-4-nitro-benzoate (450 mg, 2.03 mmol, 88% yield).

¹H NMR (400 MHz, chloroform-d) 6=8.04 (dd, J=2.3, 8.5 Hz, 1H), 7.92 (d, J=8.5 Hz, 1H), 7.86 (d, J=2.3 Hz, 1H), 3.99 (s, 3H), 2.69 (tt, J=5.4, 8.5 Hz, 1H), 1.19-1.11 (m, 2H), 0.86-0.79 (m, 2H) ppm.

### Step 2: 2-cyclopropyl-N-methyl-4-nitro-benzamide

To a solution of methyl 2-cyclopropyl-4-nitro-benzoate (350.0 mg, 1.58 mmol, 1 eq) in methanol (10 mL) was added methylamine in methanol (10.0 mL). The resulting mixture was heated to 80° C. for 15 h. The mixture was concentrated and the obtained residue was triturated with MTBE (10 mL) to give 2-cyclopropyl-N-methyl-4-nitro-benzamide (220 mg, 1 mmol, 63.14% yield). MS (ESI) m/z: 222.2 [M+H]⁺

### Step 3: 4-amino-2-cyclopropyl-N-methyl-benzamide

The title compound was obtained in analogy to step 4 in the preparation of Reference Example 450 using 2-cyclopropyl-N-methyl-4-nitro-benzamide. MS (ESI) m/z: 191.2. [M+H]⁺

### Step 4: 8-chloro-3-(4-(difluoromethoxy)phenyl)imidazo[1,2-a]pyrazine

The title compound was obtained in analogy to step 1 in the preparation of Reference Example 479 using 8-chloro-3-iodoimidazo[1,2-a]pyrazine and (4-(difluoromethoxy)phenyl)boronic acid. MS (ESI) m/z: 296.1 [M+H]⁺

### Step 5: 2-cyclopropyl-4-[[3-[4-(difluoromethoxy)phenyl]imidazo[1,2-a]pyrazin-8-yl]amino]-N-methyl-benzamide

The title compound was obtained in analogy to step 2 in the preparation of Reference Example 479 using 8-chloro-3-(4-(difluoromethoxy)phenyl)imidazo[1,2-a]pyrazine and 4-amino-2-cyclopropyl-N-methyl-benzamide as substrates. MS (ESI) m/z: 450.1. [M+H]⁺

### Reference Example 501

### N-[2-(2-aminoethoxy)ethyl]-2-(2,2-difluoroethyl)-4-[[3-(2,3-difluoro-4-methoxy-phenyl)imidazo[1,2-a]pyrazin-8-yl]amino]benzamide; Formic Acid

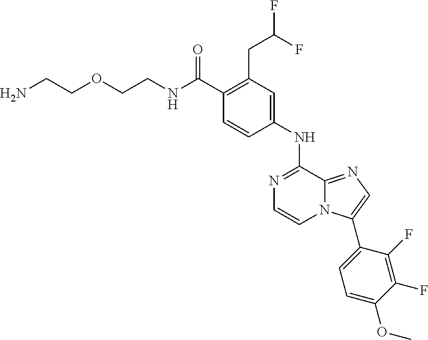 [SMILES: COc1ccc(-c2cnc3c(Nc4ccc(C(=O)NCCOCCN)c(CC(F)F)c4)nccn23)c(F)c1F]

### Step 1: methyl 2-formyl-4-nitrobenzoate

A solution of methyl 4-nitro-2-vinyl-benzoate (3.5 g, 17 mmol, 1 eq) in DCM (20 mL) was stirred under ozone (100 mL, 16.9 mmol, 1 eq) at −40° C. for 0.5 h, the mixture was quenched with dimethylsulfide (10.0 mL, 16.9 mmol, 1 eq) and then concentrated to give the desired product methyl 2-formyl-4-nitro-benzoate (2.8 g, 13 mmol, 79% yield), which was used in the next step without further purification. MS (ESI) m/z: 210.1 [M+H]⁺

### Step 2: methyl 2-(2,2-difluorovinyl)-4-nitro-benzoate

To a solution of methyl 2-formyl-4-nitro-benzoate (647.0 mg, 3.09 mmol, 1 eq) in DMF (5 mL) was added triphenylphosphine (973.6 mg, 3.71 mmol, 1.2 eq) and (2-chloro-2,2-difluoro-acetyl)oxysodium (707.41 mg, 4.64 mmol, 1.5 eq). The resulting suspension was stirred at 100° C. for 0.5 h under nitrogen. The mixture was diluted with water (100 mL), extracted with ethyl acetate (50 mL×2), washed with brine, dried over sodium sulfate and concentrated. The residue was purified by prep-TLC (PE:EA=5:1) to afford methyl 2-(2,2-difluorovinyl)-4-nitro-benzoate (130 mg, 0.530 mmol, 17.28% yield) as white solid.

¹H NMR (400 MHz, CHLOROFORM-d) δ=8.48-8.43 (m, 1H), 8.17-8.11 (m, 2H), 6.41-6.30 (m, 1H) ppm.

### Step 3: methyl 4-amino-2-(2,2-difluoroethyl)benzoate

The title compound was obtained in analogy to step 4 in the preparation of Reference Example 450 using methyl 2-(2,2-difluorovinyl)-4-nitro-benzoate. MS (ESI) m/z: 216.1 [M+H]⁺

### Step 4: methyl 2-(2,2-difluoroethyl)-4-[[3-(2,3-difluoro-4-methoxy-phenyl)imidazo[1,2-a]pyrazin-8-yl]amino]benzoate

The title compound was obtained in analogy to step 2 in the preparation of Reference Example 479 using methyl 4-amino-2-(2,2-difluoroethyl)benzoate and 1-chloro-6-(2,3-difluoro-4-methoxy-phenyl)pyrrolo[1,2-a]pyrazine. MS (ESI) m/z: 475.2 [M+H]⁺

### Step 5: 2-(2,2-difluoroethyl)-4-[[3-(2,3-difluoro-4-methoxy-phenyl)imidazo[1,2-a]pyrazin-8-yl]amino]benzoic Acid

To a solution of methyl 2-(2,2-difluoroethyl)-4-[[3-(2,3-difluoro-4-methoxy-phenyl)imidazo[1,2-a]pyrazin-8-yl]amino]benzoate (60.0 mg, 0.130 mmol, 1 eq) in THF (3 mL) and water (1 mL) was added lithium hydroxide monohydrate (6.37 mg, 0.150 mmol, 1.2 eq). The mixture was acidified with 1N aq. HCl to pH=3 and extracted with ethyl acetate (50 mL), washed with brine, dried over sodium sulfate and concentrated to afford 2-(2,2-difluoroethyl)-4-[[3-(2,3-difluoro-4-methoxy-phenyl)imidazo[1,2-a]pyrazin-8-yl]amino]benzoic acid (50 mg, 0.110 mmol, 85% yield) as white solid. MS (ESI) m/z: 461.1 [M+H]⁺

### Step 6: tert-butyl N-[2-[2-[[2-(2,2-difluoroethyl)-4-[[3-(2,3-difluoro-4-methoxy-phenyl)imidazo[1,2-a]pyrazin-8-yl]amino]benzoyl]amino]ethoxy]ethyl]carbamate

The title compound was obtained in analogy to step 3 in the preparation of Reference Example 479 using 2-(2,2-difluoroethyl)-4-[[3-(2,3-difluoro-4-methoxy-phenyl)imidazo[1,2-a]pyrazin-8-yl]amino]benzoic acid and N-BOC-2-(2-amino-ethoxy)-ethylamine for this condensation. MS (ESI) m/z: 647.4 [M+H]⁺

### Step 7: N-[2-(2-aminoethoxy)ethyl]-2-(2,2-difluoroethyl)-4-[[3-(2,3-difluoro-4-methoxy-phenyl)imidazo[1,2-a]pyrazin-8-yl]amino]benzamide

The title compound was obtained in analogy to step 2 in the preparation of Reference Example 10 using tert-butyl N-[2-[2-[[2-(2,2-difluoroethyl)-4-[[3-(2,3-difluoro-4-methoxy-phenyl)imidazo[1,2-a]pyrazin-8-yl]amino]benzoyl]amino]ethoxy]ethyl]carbamate as substrate. MS (ESI) m/z: 547.2 [M+H]⁺

### Reference Example 502

### 4-[[3-[4-(difluoromethoxy)phenyl]imidazo[1,2-a]pyrazin-8-yl]amino]-2-(2-hydroxyethoxymethyl)-N-methyl-benzamide

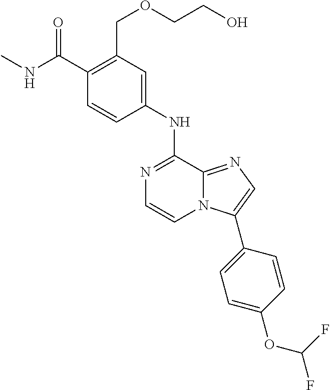 [SMILES: CNC(=O)c1ccc(Nc2nccn3c(-c4ccc(OC(F)F)cc4)cnc23)cc1COCCO]

### Step 1: 1-bromo-2-(chloromethyl)-4-nitro-benzene

A mixture of (2-bromo-5-nitro-phenyl)methanol (2.0 g, 8.62 mmol, 1 eq) and SOCl₂ (1.03 g, 8.62 mmol, 1 eq) in 1,4-dioxane (20 mL) was heated to 60° C. for 1 h. The mixture was concentrated and the residue was purified by silica gel chromatography eluting with PE:EA=20:1 to afford 1-bromo-2-(chloromethyl)-4-nitro-benzene (1.8 g, 7.19 mmol, 83% yield).

¹H NMR (400 MHz, CHLOROFORM-d) δ=8.31 (d, J=2.7 Hz, 1H), 7.99 (dd, J=2.7, 8.8 Hz, 1H), 7.72 (d, J=8.7 Hz, 1H), 4.73-4.64 (m, 2H) ppm.

### Step 2: 2-[(2-bromo-5-nitro-phenyl)methoxy]ethanol

To an ice-cooled solution of ethylene glycol (1.67 mL, 29.94 mmol, 5 eq) in THF (20 mL) and DMF (20 mL) was added sodium hydride, 60% in oil (0.36 g, 8.98 mmol, 1.5 eq). The resulting mixture was stirred for 5 min, and then 1-bromo-2-(chloromethyl)-4-nitro-benzene (1.5 g, 5.99 mmol, 1 eq) in THF (5 mL) was added. The resulting suspension was stirred at 10° C. for 15 h. The mixture was poured into saturated aq. NH₄Cl (200 mL) solution, extracted with EA (50 mL×2), washed with brine, dried over sodium sulfate and concentrated. The crude material was purified by silica gel chromatography eluting with PE:EA from 5:1 to 3:1 to afford 2-[(2-bromo-5-nitro-phenyl)methoxy]ethanol (1 g, 3.62 mmol, 60% yield).

¹H NMR (400 MHz, CHLOROFORM-d) δ=8.39 (d, J=2.8 Hz, 1H), 8.04 (dd, J=2.8, 8.7 Hz, 1H), 7.75 (d, J=8.7 Hz, 1H), 4.69 (s, 2H), 3.93-3.88 (m, 2H), 3.81-3.77 (m, 2H) ppm.

### Step 3: 2-(2-hydroxyethoxymethyl)-4-nitro-benzonitrile

A mixture of 2-[(2-bromo-5-nitro-phenyl)methoxy]ethanol (1.0 g, 3.62 mmol, 1 eq), zinc cyanide (1.28 g, 10.87 mmol, 3 eq) and tetrakis(triphenylphosphine)palladium(0) (418.56 mg, 0.360 mmol, 0.100 eq) in DMF (10 mL) was heated to 110° C. for 15 h under nitrogen protection. The mixture was diluted with water (100 mL), extracted with EA (50 mL×2), dried over sodium sulfate and concentrated. The residue was purified by silica gel chromatography eluting with PE:EA from 10:1 to 5:1 to afford 2-(2-hydroxyethoxymethyl)-4-nitro-benzonitrile (600 mg, 2.7 mmol, 74% yield).

¹H NMR (400 MHz, CHLOROFORM-d) δ=8.54-8.44 (m, 1H), 8.28 (dd, J=2.3, 8.5 Hz, 1H), 7.90 (d, J=8.4 Hz, 1H), 4.87 (s, 2H), 3.92-3.86 (m, 2H), 3.83-3.77 (m, 2H) ppm.

### Step 4: 2-(2-hydroxyethoxymethyl)-4-nitro-benzoic Acid

A mixture of 2-(2-hydroxyethoxymethyl)-4-nitro-benzonitrile (600 mg, 2.7 mmol, 1 eq) in aq. sodium hydroxide (10.0 mL, 0.270 mmol, 0.100 eq) was heated to 100° C. for 3 h. The mixture was acidified with 2 N HCl aq. to pH=4 and extracted with EA (100 mL×2), washed with brine, dried over Na₂SO₄ and concentrated. The residue was triturated with DCM to afford 2-(2-hydroxyethoxymethyl)-4-nitro-benzoic acid (450 mg, 1.87 mmol, 69.09% yield). MS (ESI) m/z: 264.0. [M+Na]⁺

### Step 5: 2-(2-hydroxyethoxymethyl)-N-methyl-4-nitro-benzamide

The title compound was obtained in analogy to step 3 in the preparation of Reference Example 479 using 2-(2-hydroxyethoxymethyl)-4-nitro-benzoic acid and methylamine (2 M in THF). MS (ESI) m/z: 277.0 [M+Na]⁺

### Step 6: 4-amino-2-(2-hydroxyethoxymethyl)-N-methyl-benzamide

The title compound was obtained in analogy to step 4 in the preparation of Reference Example 450 using 2-(2-hydroxyethoxymethyl)-N-methyl-4-nitro-benzamide. MS (ESI) m/z: 225.3 [M+H]⁺

### Step 7: 4-[[3-[4-(difluoromethoxy)phenyl]imidazo[1,2-a]pyrazin-8-yl]amino]-2-(2-hydroxyethoxymethyl)-N-methyl-benzamide

A mixture of 4-amino-2-(2-hydroxyethoxymethyl)-N-methyl-benzamide (120 mg, 0.540 mmol, 1 eq), 8-chloro-3-[4-(difluoromethoxy)phenyl]imidazo[1,2-a]pyrazine (158.21 mg, 0.540 mmol, 1 eq), Brettphos Pd G3 (45.88 mg, 0.050 mmol, 0.100 eq) and K₂CO₃ (147.91 mg, 1.07 mmol, 2 eq) was heated to 110° C. for 15 h under nitrogen. The mixture was diluted with water, and extracted with ethyl acetate (100 mL×2). The combined organic phases were washed with brine, dried over sodium sulfate and concentrated. The residue was purified by prep-TLC (DCM:MeOH=10:1) to afford 4-[[3-[4-(difluoromethoxy)phenyl]imidazo[1,2-a]pyrazin-8-yl]amino]-2-(2-hydroxyethoxymethyl)-N-methyl-benzamide (55.5 mg, 0.110 mmol, 21% yield). MS (ESI) m/z: 484.0 [M+H]⁺

### Reference Example 495

### 2-(3-aminopropyl)-6-[[3-(2,3-difluoro-4-methoxy-phenyl)imidazo[1,2-a]pyrazin-8-yl]amino]-3,4-dihydroisoquinolin-1-one

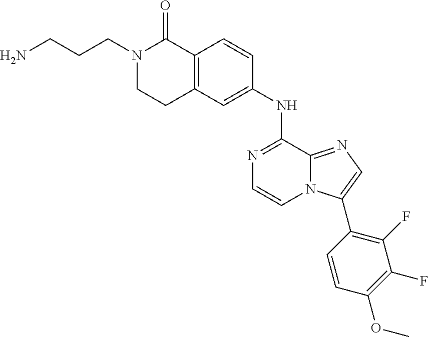 [SMILES: COc1ccc(-c2cnc3c(Nc4ccc5c(c4)CCN(CCCN)C5=O)nccn23)c(F)c1F]

### Step 1: 6-bromo-2-(3-tetrahydropyran-2-yloxypropyl)-3,4-dihydroisoquinolin-1-one

To a mixture of 6-bromo-3,4-dihydro-2H-isoquinolin-1-one (400 mg, 1.77 mmol, 1 eq) in DMF (10 mL) was added sodium hydride (141.55 mg, 3.54 mmol, 2 eq) at 0° C. Then the mixture was stirred at 0° C. for 0.5 h. Then 2-(3-bromopropoxy)tetrahydro-2H-pyran (1184.29 mg, 5.31 mmol, 3 eq) was added to the mixture at 25° C. and the mixture was stirred at 25° C. for another 15.5 h. Then the mixture was poured into water (30.0 mL) and extracted with ethyl acetate (50.0 mL*2). The organic phase was dried and concentrated in vacuo to give the crude product as brown oil. The crude product was purified by silica gel column chromatography eluting with PE/EA from 20:1 to 2:1 to give 6-bromo-2-(3-tetrahydropyran-2-yloxypropyl)-3,4-dihydroisoquinolin-1-one (500 mg, 1.36 mmol, 76.73% yield) as yellow oil. MS (ESI, m/z): 284.0 [M-84+H]⁺, 286.1 [M-84+2+H]⁺

### Step 2: 3-(2,3-difluoro-4-methoxyphenyl)-N-(4-methoxybenzyl)imidazo[1,2-a]pyrazin-8-amine

The title compound was obtained in analogy to step 2 in the preparation of Reference Example 479 using 8-chloro-3-(2,3-difluoro-4-methoxyphenyl)imidazo[1,2-a]pyrazine and (4-methoxyphenyl)methanamine. MS (ESI) m/z: 397.2 [M+H]⁺

### Step 3: 3-(2,3-difluoro-4-methoxyphenyl)imidazo[1,2-a]pyrazin-8-amine

To a stirred solution of 3-(2,3-difluoro-4-methoxyphenyl)-N-(4-methoxybenzyl)imidazo[1,2-a]pyrazin-8-amine (2 g, 5 mmol, 1 eq) in DCM (10 mL) was added TFA (10 mL). The reaction mixture was stirred at 30° C. for 16 h The mixture was concentrated under reduced pressure to give 1.5 g of the crude product, used directly in the next step without further purification.

### Step 4: 6-[[3-(2,3-difluoro-4-methoxy-phenyl)imidazo[1,2-a]pyrazin-8-yl]amino]-2-(3-tetrahydropyran-2-yloxypropyl)-3,4-dihydroisoquinolin-1-one

To a mixture of 6-bromo-2-(3-tetrahydropyran-2-yloxypropyl)-3,4-dihydroisoquinolin-1-one (440.16 mg, 1.2 mmol, 1.1 eq) and 3-(2,3-difluoro-4-methoxy-phenyl)imidazo[1,2-a]pyrazin-8-amine (300.0 mg, 1.09 mmol, 1 eq) in tert-butanol (10 mL) was added potassium carbonate (300 mg, 2.17 mmol, 2 eq) and BrettPhos-Pd-G3 (197.0 mg, 0.220 mmol, 0.200 eq) at 25° C. Then the mixture was stirred at 110° C. for 16 h. Then the mixture was poured into water (30.0 mL) and extracted with ethyl acetate (50.0 mL*2). The organic phase was dried and concentrated in vacuo to give the crude product as brown solid. The crude product was triturated with ethyl acetate (20.0 mL) to give 6-[[3-(2,3-difluoro-4-methoxy-phenyl)imidazo[1,2-a]pyrazin-8-yl]amino]-2-(3-tetrahydropyran-2-yloxypropyl)-3,4-dihydroisoquinolin-1-one (400 mg, 0.710 mmol, 65.32% yield) as white solid. MS (ESI) m/z: 564.3 [M+H]⁺

### Step 5: 6-((3-(2,3-difluoro-4-methoxyphenyl)imidazo[1,2-a]pyrazin-8-yl)amino)-2-(3-hydroxypropyl)-3,4-dihydroisoquinolin-1(2H)-one

A mixture of 6-[[3-(2,3-difluoro-4-methoxy-phenyl)imidazo[1,2-a]pyrazin-8-yl]amino]-2-(3-tetrahydropyran-2-yloxypropyl)-3,4-dihydroisoquinolin-1-one (400.0 mg, 0.710 mmol, 1 eq) in HCl/MeOH (10.0 mL, 40 mmol, 56.36 eq) was stirred at 25° C. for 16 h. Then the mixture was concentrated in vacuo to give the crude product as grey solid. The crude product was purified by prep-HPLC (FA) to give 250 mg desired product as white solid. MS (ESI) m/z: 480.2 [M+H]⁺

### Step 6: 3-[6-[[3-(2,3-difluoro-4-methoxy-phenyl)imidazo[1,2-a]pyrazin-8-yl]amino]-1-oxo-3,4-dihydroisoquinolin-2-yl]propyl methanesulfonate

The title compound was obtained in analogy to step 4 in the preparation of Reference Example 482 using 6-[[3-(2,3-difluoro-4-methoxy-phenyl)imidazo[1,2-a]pyrazin-8-yl]amino]-2-(3-hydroxypropyl)-3,4-dihydroisoquinolin-1-one as starting material. MS (ESI) m/z: 558.2 [M+H]⁺

### Step 7: 2-(3-aminopropyl)-6-[[3-(2,3-difluoro-4-methoxy-phenyl)imidazo[1,2-a]pyrazin-8-yl]amino]-3,4-dihydroisoquinolin-1-one

To a mixture of NH₃ in THF (2.0 mL, 8 mmol, 111.51 eq) was added 3-[6-[[3-(2,3-difluoro-4-methoxy-phenyl)imidazo[1,2-a]pyrazin-8-yl]amino]-1-oxo-3,4-dihydroisoquinolin-2-yl]propyl methanesulfonate (40.0 mg, 0.070 mmol, 1 eq) at −78° C. Then the mixture was stirred at −78° C. for 5 h. Then the mixture was concentrated in vacuo and purified by prep-HPLC to give 2-(3-aminopropyl)-6-[[3-(2,3-difluoro-4-methoxy-phenyl)imidazo[1,2-a]pyrazin-8-yl]amino]-3,4-dihydroisoquinolin-1-one (14 mg, 0.030 mmol, 40% yield) as a white solid. MS (ESI) m/z: 479.3 [M+H]⁺

The following additional Examples have been prepared with the methods described above:

| | | | ESI MS |
|---|---|---|---|
| Ex. | Name | Structure | [M + H]⁺ |
| 51 | [4-(aminomethyl)piperidin-1- yl]-[4-[[3-[4- (difluoromethoxy)phenyl] imidazo[1,2-a]pyrazin-8- yl]amino]-2- iodophenyl]methanone; hydrochloride | 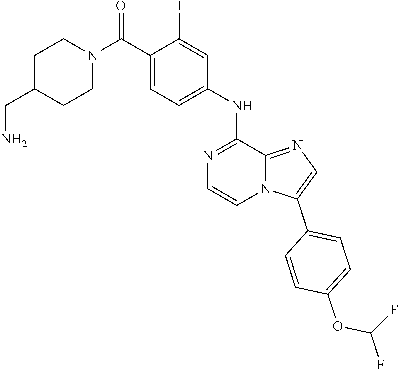 [SMILES: NCC1CCN(C(=O)c2ccc(Nc3nccn4c(-c5ccc(OC(F)F)cc5)cnc34)cc2I)CC1] | 619.2 |
| 52 | [4-[2- (aminomethyl)morpholine-4- carbonyl]piperidin-1-yl]-[2- chloro-4-[[3-(3-fluoro-4- methoxyphenyl)imidazo[1,2- a]pyrazin-8- yl]amino]phenyl]methanone; hydrochloride | 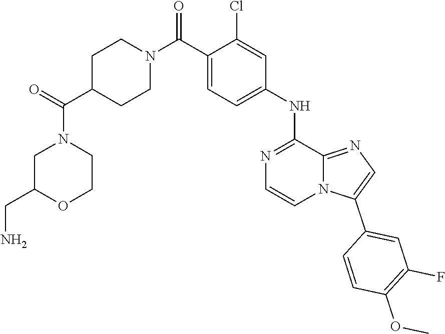 [SMILES: COc1ccc(-c2cnc3c(Nc4ccc(C(=O)N5CCC(C(=O)N6CCOC(CN)C6)CC5)c(Cl)c4)nccn23)cc1F] | 622.2 |
| 53 | 1-[4-[2-chloro-4-[[3-(2,3- difluoro-4- methoxyphenyl)imidazo[1,2- a]pyrazin-8- yl]amino]benzoyl]piperazin-1- yl]-2- (methylamino)ethanone; hydrochloride | 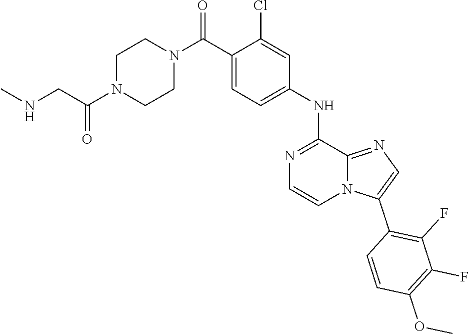 [SMILES: CNCC(=O)N1CCN(C(=O)c2ccc(Nc3nccn4c(-c5ccc(OC)c(F)c5F)cnc34)cc2Cl)CC1] | 570.2 |
| 54 | N-(azetidin-3-ylmethyl)-1-[2- chloro-4-[[3-(3-fluoro-4- methoxyphenyl)imidazo[1,2- a]pyrazin-8- yl]amino]benzoyl]piperidine- 4-carboxamide;hydrochloride | 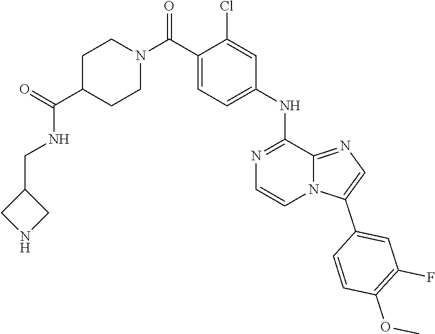 [SMILES: COc1ccc(-c2cnc3c(Nc4ccc(C(=O)N5CCC(C(=O)NCC6CNC6)CC5)c(Cl)c4)nccn23)cc1F] | 592.2 |
| 55 | [2-chloro-4-[[3-(3-fluoro-4- methoxyphenyl)imidazo[1,2- a]pyrazin-8-yl]amino]phenyl]- [4-[2- (dimethylamino)ethyl]piperazin-1- yl]methanone;hydrochloride | 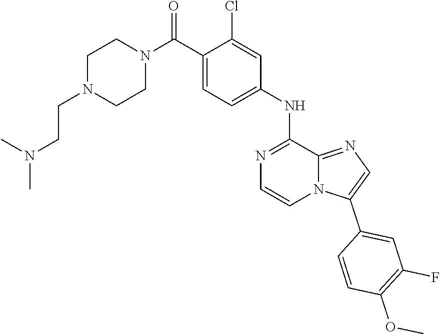 [SMILES: COc1ccc(-c2cnc3c(Nc4ccc(C(=O)N5CCN(CCN(C)C)CC5)c(Cl)c4)nccn23)cc1F] | 550.5 [M − H]− |
| 56 | 3-amino-1-[4-[2-chloro-4-[[3- (3-fluoro-4- methoxyphenyl)imidazo[1,2- a]pyrazin-8- yl]amino]benzoyl]piperazin-1- yl]-2-hydroxypropan-1- one; hydrochloride | 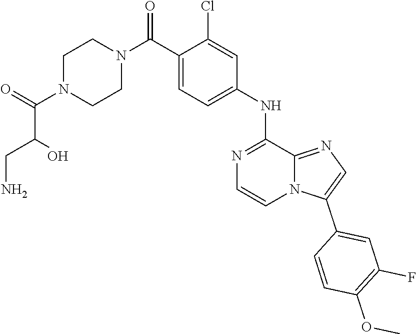 [SMILES: COc1ccc(-c2cnc3c(Nc4ccc(C(=O)N5CCN(C(=O)C(O)CN)CC5)c(Cl)c4)nccn23)cc1F] | 568.2 |
| 57 | [4-[2-(azetidin-1- yl)ethyl]piperazin-1-yl]-[2- chloro-4-[[3-(3-fluoro-4- methoxyphenyl)imidazo[1,2- a]pyrazin-8- yl]amino]phenyl]methanone | 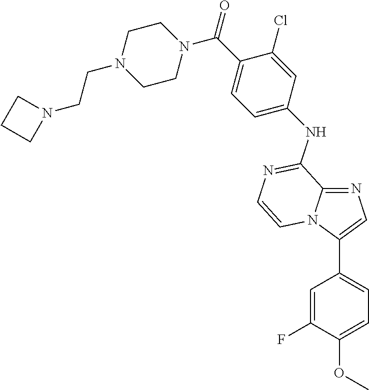 [SMILES: COc1ccc(-c2cnc3c(Nc4ccc(C(=O)N5CCN(CCN6CCC6)CC5)c(Cl)c4)nccn23)cc1F] | 564.2 |
| 58 | [2-chloro-4-[[3-(3-fluoro-4- methoxyphenyl)imidazo[1,2- a]pyrazin-8-yl]amino]phenyl]- [4-[2- (cyclopropylamino)ethyl] piperazin-1-yl]methanone | 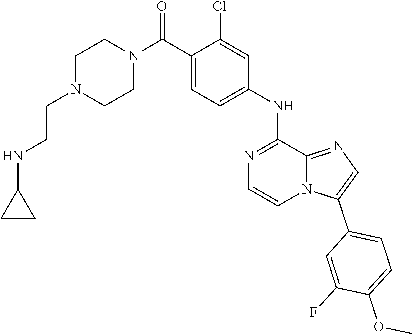 [SMILES: COc1ccc(-c2cnc3c(Nc4ccc(C(=O)N5CCN(CCNC6CC6)CC5)c(Cl)c4)nccn23)cc1F] | 564.2 |
| 59 | 4-[1-[2-chloro-4-[[3-(3-fluoro- 4- methoxyphenyl)imidazo[1,2- a]pyrazin-8- yl]amino]benzoyl]piperidine- 4-carbonyl]piperazine-2- carboxylic acid | 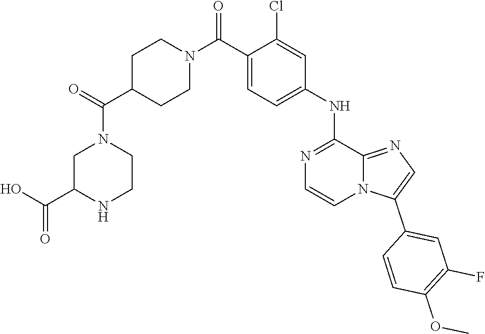 [SMILES: COc1ccc(-c2cnc3c(Nc4ccc(C(=O)N5CCC(C(=O)N6CCNC(C(=O)O)C6)CC5)c(Cl)c4)nccn23)cc1F] | 636.2 |
| 60 | N-(3-amino-2- hydroxypropyl)-1-[2-chloro-4- [[3-(3-fluoro-4- methoxyphenyl)imidazo[1,2- a]pyrazin-8- yl]amino]benzoyl]piperidine- 4-carboxamide;hydrochloride | 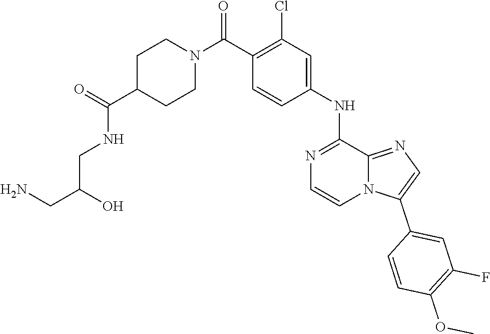 [SMILES: COc1ccc(-c2cnc3c(Nc4ccc(C(=O)N5CCC(C(=O)NCC(O)CN)CC5)c(Cl)c4)nccn23)cc1F] | 596.3 |
| 61 | [4-[[3-(4- methoxyphenyl)imidazo[1,2- a]pyrazin-8-yl]amino]phenyl]- morpholin-4-ylmethanone | 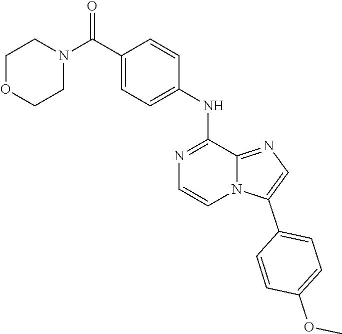 [SMILES: COc1ccc(-c2cnc3c(Nc4ccc(C(=O)N5CCOCC5)cc4)nccn23)cc1] | 429 (M − H)⁻ |
| 62 | [4-[[3-(3-chloro-4- methoxyphenyl)imidazo[1,2- a]pyrazin-8-yl]amino]phenyl]- (4-hydroxypiperidin-1- yl)methanone | 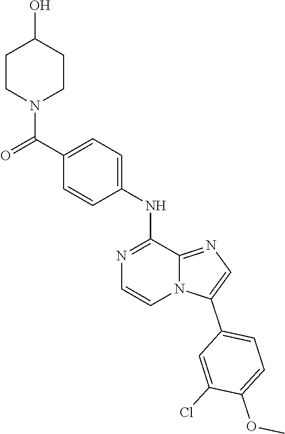 [SMILES: COc1ccc(-c2cnc3c(Nc4ccc(C(=O)N5CCC(O)CC5)cc4)nccn23)cc1Cl] | 478.2 |
| 63 | [4-(2-hydroxyethyl)piperazin- 1-yl]-[4-[[3-(4- methoxyphenyl)imidazo[1,2- a]pyrazin-8- yl]amino]phenyl]methanone | 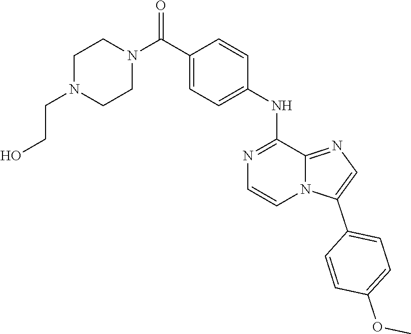 [SMILES: COc1ccc(-c2cnc3c(Nc4ccc(C(=O)N5CCN(CCO)CC5)cc4)nccn23)cc1] | 472 (M − H)⁻ |
| 64 | [4-[[3-(4- methoxyphenyl)imidazo[1,2- a]pyrazin-8-yl]amino]phenyl]- (4-methylpiperazin-1- yl)methanone | 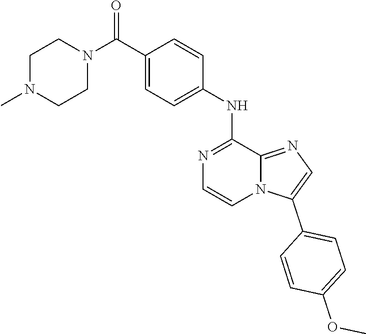 | 442 |
| 65 | [4-[[3-(3-chloro-4- methoxyphenyl)imidazo[1,2- a]pyrazin-8-yl]amino]phenyl]- piperidin-1-ylmethanone | 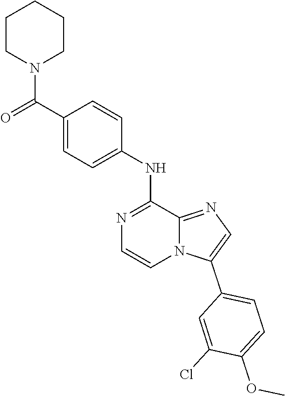 [SMILES: COc1ccc(-c2cnc3c(Nc4ccc(C(=O)N5CCCCC5)cc4)nccn23)cc1Cl] | 462.1 |
| 66 | [3-(dimethylamino)pyrrolidin- 1-yl]-[4-[[3-(4- methoxyphenyl)imidazo[1,2- a]pyrazin-8- yl]amino]phenyl]methanone | 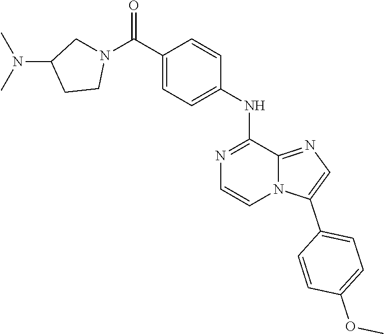 | 456 |
| 67 | [2-chloro-4-[[3-[4- (difluoromethoxy)phenyl]imidazo [1,2-a]pyrazin-8- yl]amino]phenyl]-(4- piperazin-1-ylpiperidin-1- yl)methanone;hydrochloride | 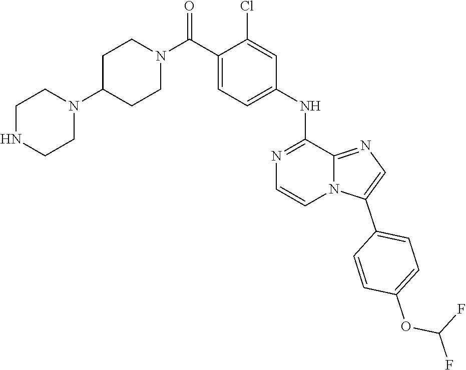 [SMILES: O=C(c1ccc(Nc2nccn3c(-c4ccc(OC(F)F)cc4)cnc23)cc1Cl)N1CCC(N2CCNCC2)CC1] | 583.2 |
| 68 | [2-chloro-4-[[3-(2-chloro-3- fluoro-4- methoxyphenyl)imidazo[1,2- a]pyrazin-8-yl]amino]phenyl]- [4-[2- (dimethylamino)ethyl]piperazin- 1-yl]methanone | 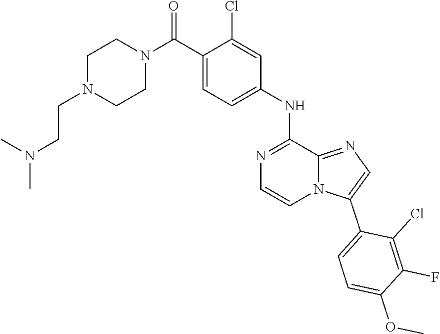 [SMILES: COc1ccc(-c2cnc3c(Nc4ccc(C(=O)N5CCN(CCN(C)C)CC5)c(Cl)c4)nccn23)c(Cl)c1F] | 587.4 |
| 69 | [2-chloro-4-[[3-[4- (difluoromethoxy)phenyl] imidazo[1,2-a]pyrazin-8- yl]amino]phenyl]-[4-[2- (dimethylamino)ethyl]piperazin- 1-yl]methanone | 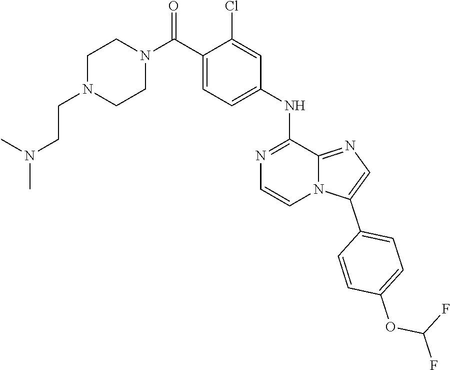 [SMILES: CN(C)CCN1CCN(C(=O)c2ccc(Nc3nccn4c(-c5ccc(OC(F)F)cc5)cnc34)cc2Cl)CC1] | 571.3 |
| 70 | [2-chloro-4-[[3-(3-fluoro-4- methoxyphenyl)imidazo[1,2- a]pyrazin-8-yl]amino]phenyl]- [4-[3- (hydroxymethyl)piperazine-1- carbonyl]piperidin-1- yl]methanone;hydrochloride | 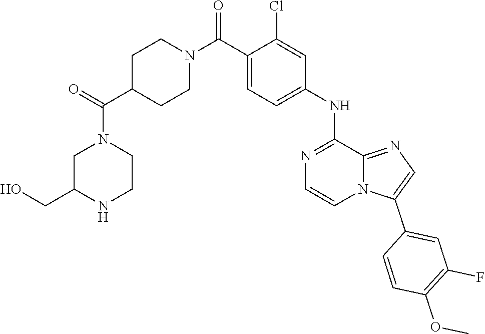 [SMILES: COc1ccc(-c2cnc3c(Nc4ccc(C(=O)N5CCC(C(=O)N6CCNC(CO)C6)CC5)c(Cl)c4)nccn23)cc1F] | 623.4 |
| 71 | [2-chloro-4-[[3-(3-fluoro-4- methoxyphenyl)imidazo[1,2- a]pyrazin-8-yl]amino]phenyl]- (4-pyrrolidin-3-ylpiperazin-1- yl)methanone;hydrochloride | 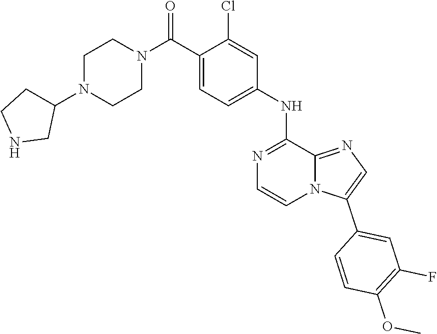 [SMILES: COc1ccc(-c2cnc3c(Nc4ccc(C(=O)N5CCN(C6CCNC6)CC5)c(Cl)c4)nccn23)cc1F] | 551.3 |
| 72 | [2-chloro-4-[[3-(3-fluoro-4- methoxyphenyl)imidazo[1,2- a]pyrazin-8-yl]amino]phenyl]- [4-(1H-imidazol-5- ylmethyl)piperazin-1- yl]methanone | 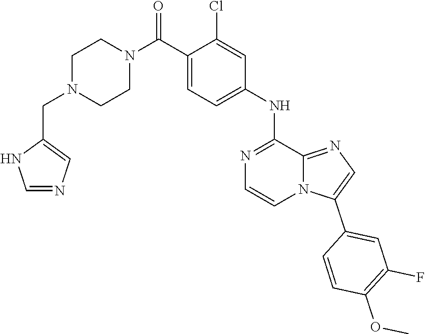 [SMILES: COc1ccc(-c2cnc3c(Nc4ccc(C(=O)N5CCN(Cc6cnc[nH]6)CC5)c(Cl)c4)nccn23)cc1F] | 562.4 |
| 73 | [4-(azetidin-3-yl)piperazin-1- yl]-[2-chloro-4-[[3-(3-fluoro- 4- methoxyphenyl)imidazo[1,2- a]pyrazin-8- yl]amino]phenyl]methanone; hydrochloride | 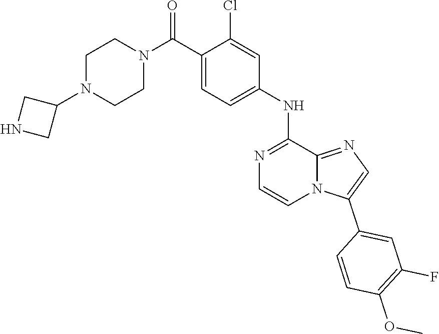 [SMILES: COc1ccc(-c2cnc3c(Nc4ccc(C(=O)N5CCN(C6CNC6)CC5)c(Cl)c4)nccn23)cc1F] | 537.2 |
| 74 | [4-[[3-(3-chloro-4- methoxyphenyl)imidazo[1,2- a]pyrazin-8-yl]amino]phenyl]- (4-methylpiperazin-1- yl)methanone | 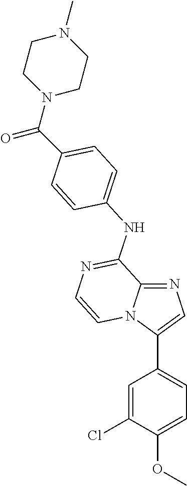 [SMILES: COc1ccc(-c2cnc3c(Nc4ccc(C(=O)N5CCN(C)CC5)cc4)nccn23)cc1Cl] | 478.2 |
| 75 | [4-(aminomethyl)piperidin-1- yl]-[2-chloro-4-[[3-(2,3- difluoro-4-prop-2- ynoxyphenyl)imidazo[1,2- a]pyrazin-8- yl]amino]phenyl]methanone | 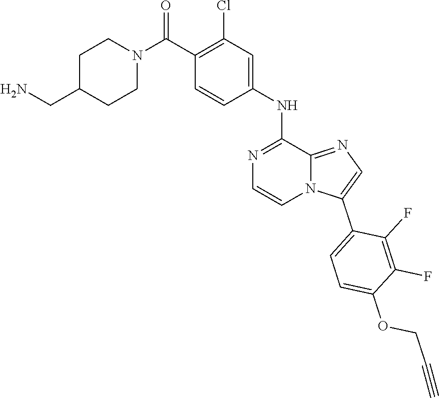 [SMILES: C#CCOc1ccc(-c2cnc3c(Nc4ccc(C(=O)N5CCC(CN)CC5)c(Cl)c4)nccn23)c(F)c1F] | 551.3 |
| 76 | [4-(aminomethyl)piperidin-1- yl]-[2-chloro-4-[[3-[3-fluoro- 4-(4-hydroxybut-2- ynoxy)phenyl]imidazo[1,2- a]pyrazin-8- yl]amino]phenyl]methanone | 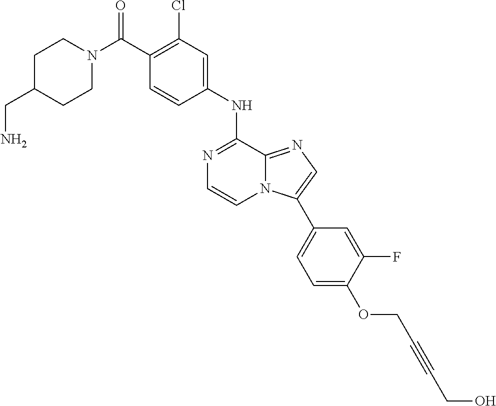 [SMILES: NCC1CCN(C(=O)c2ccc(Nc3nccn4c(-c5ccc(OCC#CCO)c(F)c5)cnc34)cc2Cl)CC1] | 562.9 |
| 77 | 2-[4-[8-[3-chloro-4-[4-[2- (methylamino)acetyl] piperazine-1- carbonyl]anilino]imidazo[1,2- a]pyrazin-3-yl]-2,3- difluorophenoxy]acetonitrile | 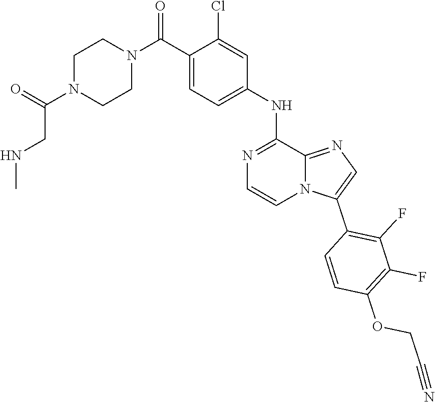 [SMILES: CNCC(=O)N1CCN(C(=O)c2ccc(Nc3nccn4c(-c5ccc(OCC#N)c(F)c5F)cnc34)cc2Cl)CC1] | 595.4 |
| 78 | 2-[4-[8-[3-bromo-4-[4-[2- (dimethylamino)ethyl] piperazine-1- carbonyl]anilino]imidazo[1,2- a]pyrazin-3-yl]-2,3- difluorophenoxy]acetonitrile; formic acid | 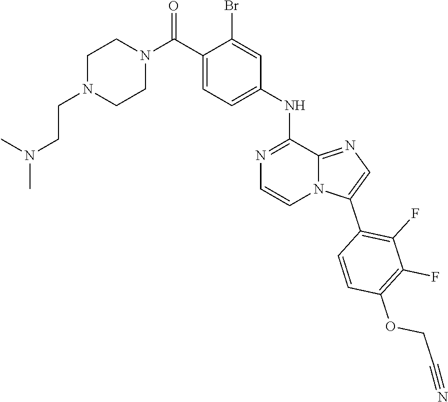 [SMILES: CN(C)CCN1CCN(C(=O)c2ccc(Nc3nccn4c(-c5ccc(OCC#N)c(F)c5F)cnc34)cc2Br)CC1] | 638.9 |
| 79 | 2-[4-[8-[3-chloro-4- (morpholine-4- carbonyl)anilino]imidazo[1,2- a]pyrazin-3-yl]-2,3- difluorophenoxy]acetonitrile | 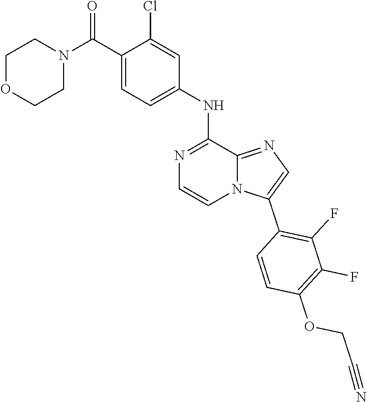 [SMILES: N#CCOc1ccc(-c2cnc3c(Nc4ccc(C(=O)N5CCOCC5)c(Cl)c4)nccn23)c(F)c1F] | 525 |
| 80 | [4-(aminomethyl)piperidin-1- yl]-[2-bromo-4-[[3-(3-fluoro- 4- methoxyphenyl)imidazo[1,2- a]pyrazin-8- yl]amino]phenyl]methanone | 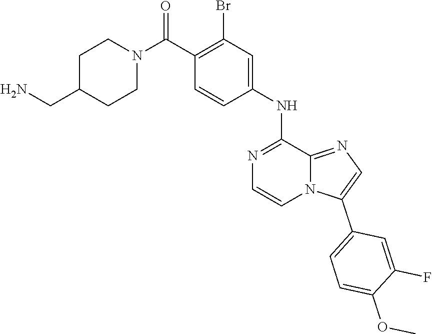 [SMILES: COc1ccc(-c2cnc3c(Nc4ccc(C(=O)N5CCC(CN)CC5)c(Br)c4)nccn23)cc1F] | 553.1 |
| 81 | [2-bromo-4-[[3-(3-fluoro-4- methoxyphenyl)imidazo[1,2- a]pyrazin-8-yl]amino]phenyl]- [4-[2- (dimethylamino)ethyl]piperazin- 1-yl]methanone;formic acid | 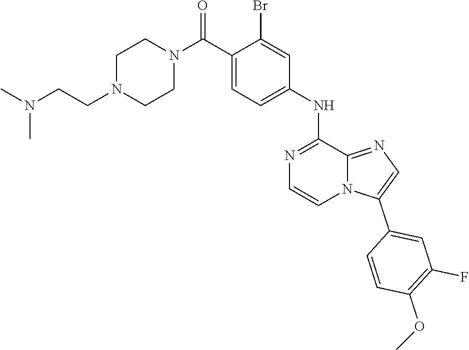 [SMILES: COc1ccc(-c2cnc3c(Nc4ccc(C(=O)N5CCN(CCN(C)C)CC5)c(Br)c4)nccn23)cc1F] | 596.0 |
| 82 | (2S)-4-[4-[4-[[3-[4- (difluoromethoxy)phenyl]imidazo [1,2-a]pyrazin-8- yl]amino]-2- methylbenzoyl]piperazine-1- carbonyl]piperazine-2- carboxylic acid;hydrochloride | 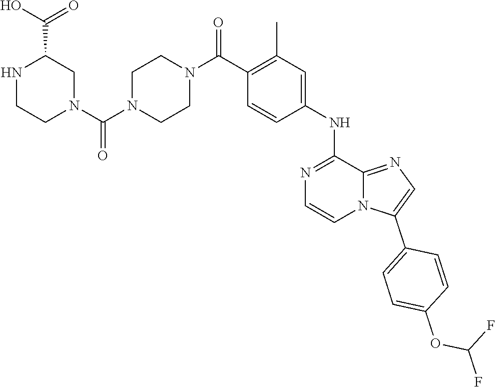 [SMILES: Cc1cc(Nc2nccn3c(-c4ccc(OC(F)F)cc4)cnc23)ccc1C(=O)N1CCN(C(=O)N2CCN[C@H](C(=O)O)C2)CC1] | 635.3 |
| 83 | piperazin-2-ylmethyl 1-[2- chloro-4-[[3-[4- (cyanomethoxy)-2,3- difluorophenyl]imidazo[1,2- a]pyrazin-8- yl]amino]benzoyl]piperidine- 4-carboxylate;2,2,2- trifluoroacetic acid | 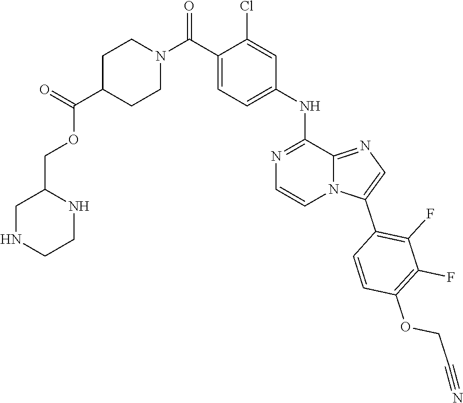 [SMILES: N#CCOc1ccc(-c2cnc3c(Nc4ccc(C(=O)N5CCC(C(=O)OCC6CNCCN6)CC5)c(Cl)c4)nccn23)c(F)c1F] | 665.3 |
| 84 | 2-[4-[8-[3-chloro-4-[4-[2- (dimethylamino)ethyl] piperazine-1- carbonyl]anilino]imidazo[1,2- a]pyrazin-3-yl]-2,3- difluorophenoxy]acetonitrile; 2,2,2-trifluoroacetic acid | 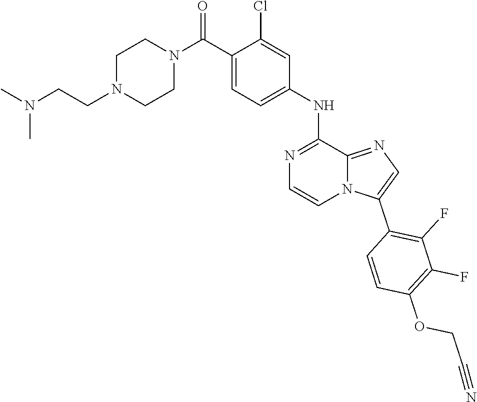 [SMILES: CN(C)CCN1CCN(C(=O)c2ccc(Nc3nccn4c(-c5ccc(OCC#N)c(F)c5F)cnc34)cc2Cl)CC1] | 595.3 |
| 85 | 2-[4-[8-[3-chloro-4-[4-[(3R)- 3-(hydroxymethyl)piperazine- 1-carbonyl]piperidine-1- carbonyl]anilino]imidazo[1,2- a]pyrazin-3-yl]-2,3- difluorophenoxy]acetonitrile; formic acid | 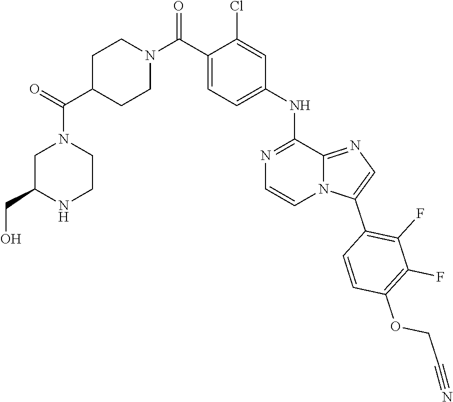 [SMILES: N#CCOc1ccc(-c2cnc3c(Nc4ccc(C(=O)N5CCC(C(=O)N6CCN[C@@H](CO)C6)CC5)c(Cl)c4)nccn23)c(F)c1F] | 665.5 |
| 86 | 2-[3-chloro-4-[8-[3-chloro-4- [4-[(3R)-3- (hydroxymethyl)piperazine-1- carbonyl]piperidine-1- carbonyl]anilino]imidazo[1,2- a]pyrazin-3-yl]-2- fluorophenoxy]acetonitrile; formic acid | 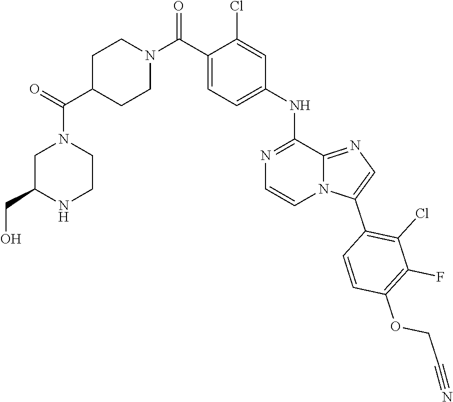 [SMILES: N#CCOc1ccc(-c2cnc3c(Nc4ccc(C(=O)N5CCC(C(=O)N6CCN[C@@H](CO)C6)CC5)c(Cl)c4)nccn23)c(Cl)c1F] | 681.3 |
| 87 | 2-[4-[8-[3-chloro-4-[4-[(3S)- 3-(hydroxymethyl)piperazine- 1-carbonyl]piperidine-1- carbonyl]anilino]imidazo[1,2- a]pyrazin-3-yl]-2,3- difluorophenoxy]acetonitrile; formic acid | 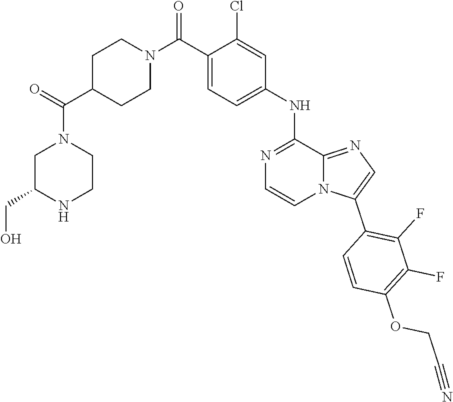 [SMILES: N#CCOc1ccc(-c2cnc3c(Nc4ccc(C(=O)N5CCC(C(=O)N6CCN[C@H](CO)C6)CC5)c(Cl)c4)nccn23)c(F)c1F] | 665.3 |
| 88 | 2-[3-chloro-4-[8-[3-chloro-4- [4-[(3S)-3- (hydroxymethyl)piperazine-1- carbonyl]piperidine-1- carbonyl]anilino]imidazo[1,2- a]pyrazin-3-yl]-2- fluorophenoxy]acetonitrile; formic acid | 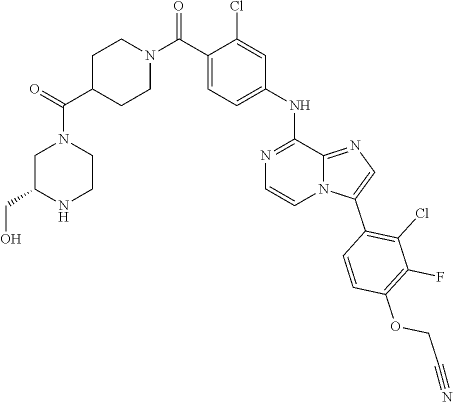 [SMILES: N#CCOc1ccc(-c2cnc3c(Nc4ccc(C(=O)N5CCC(C(=O)N6CCN[C@H](CO)C6)CC5)c(Cl)c4)nccn23)c(Cl)c1F] | 681.4 |
| 89 | 1-[2-[4-[2-chloro-4-[3-[4- (cyanomethoxy)-2,3- difluorophenyl]imidazo[1,2- a]pyrazin-8- yl]amino]benzoyl]piperazin-1- yl]ethyl]pyrrolidine-3- carboxamide;2,2,2- trifluoroacetic acid | 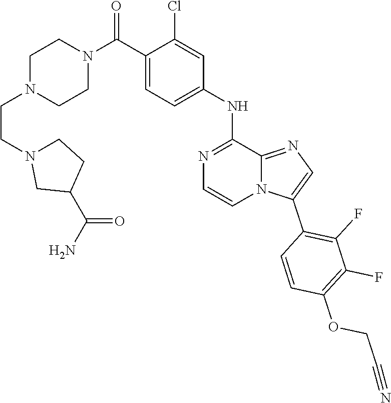 [SMILES: N#CCOc1ccc(-c2cnc3c(Nc4ccc(C(=O)N5CCN(CCN6CCC(C(N)=O)C6)CC5)c(Cl)c4)nccn23)c(F)c1F] | 664.1 |
| 90 | 2-[4-[8-[3-chloro-4-[4-[2-[3- (hydroxymethyl)pyrrolidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 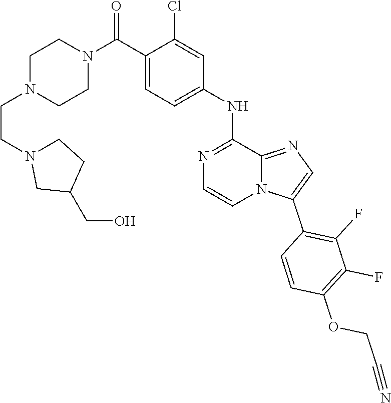 [SMILES: N#CCOc1ccc(-c2cnc3c(Nc4ccc(C(=O)N5CCN(CCN6CCC(CO)C6)CC5)c(Cl)c4)nccn23)c(F)c1F] | 651.1 |
| 91 | 2-[4-[8-[3-chloro-4-[4-[2- (methylamino)ethyl] piperazine-1- carbonyl]anilino]imidazo[1,2- a]pyrazin-3-yl]-2,3- difluorophenoxy]acetonitrile; 2,2,2-trifluoroacetic acid | 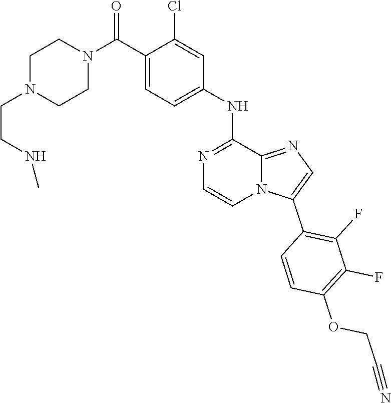 [SMILES: CNCCN1CCN(C(=O)c2ccc(Nc3nccn4c(-c5ccc(OCC#N)c(F)c5F)cnc34)cc2Cl)CC1] | 581.1 |
| 92 | 2-[4-[8-[3-chloro-4-[4-[2-[3- (hydroxymethyl)azetidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 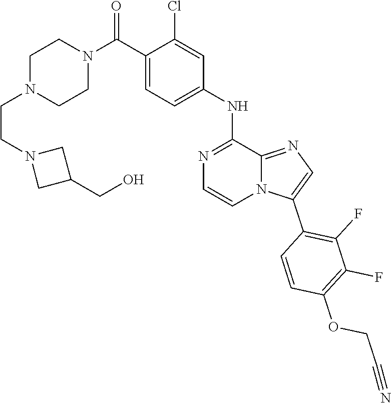 [SMILES: N#CCOc1ccc(-c2cnc3c(Nc4ccc(C(=O)N5CCN(CCN6CC(CO)C6)CC5)c(Cl)c4)nccn23)c(F)c1F] | 637 |
| 93 | 2-[4-[8-[3-chloro-4-[4-[2-[2- (hydroxymethyl)pyrrolidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 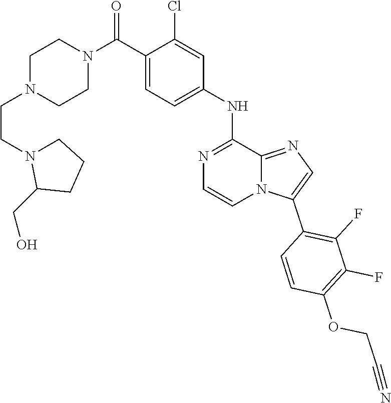 [SMILES: N#CCOc1ccc(-c2cnc3c(Nc4ccc(C(=O)N5CCN(CCN6CCCC6CO)CC5)c(Cl)c4)nccn23)c(F)c1F] | 651.4 |
| 94 | 1-[2-[4-[2-chloro-4-[[3-[4- (cyanomethoxy)-2,3- difluorophenyl]imidazo[1,2- a]pyrazin-8- yl]amino]benzoyl]piperazin-1- yl]ethyl]azetidine-3- carboxamide;2,2,2- trifluoroacetic acid | 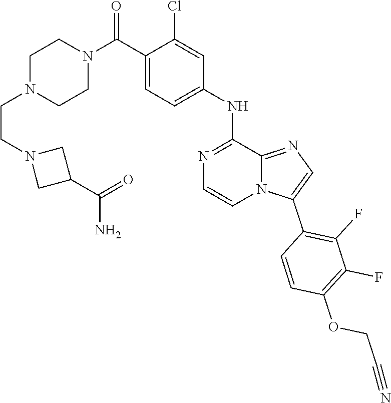 [SMILES: N#CCOc1ccc(-c2cnc3c(Nc4ccc(C(=O)N5CCN(CCN6CC(C(N)=O)C6)CC5)c(Cl)c4)nccn23)c(F)c1F] | 650.1 |
| 95 | 2-[4-[8-[3-chloro-4-[4-[2-(3- hydroxypyrrolidin-1- yl)ethyl]piperazine-1- carbonyl]anilino]imidazo[1,2- a]pyrazin-3-yl]-2,3- difluorophenoxy]acetonitrile; 2,2,2-trifluoroacetic acid | 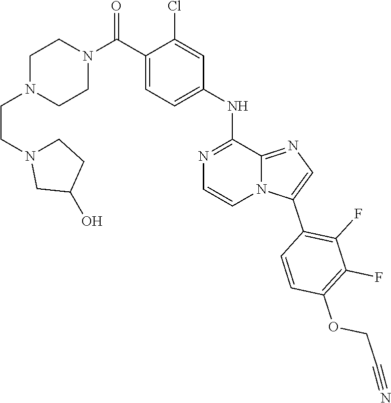 [SMILES: N#CCOc1ccc(-c2cnc3c(Nc4ccc(C(=O)N5CCN(CCN6CCC(O)C6)CC5)c(Cl)c4)nccn23)c(F)c1F] | 637.1 |
| 96 | 2-[4-[8-[3-chloro-4-[4-[2-(3- hydroxyazetidin-1- yl)ethyl]piperazine-1- carbonyl]anilino]imidazo[1,2- a]pyrazin-3-yl]-2,3- difluorophenoxy]acetonitrile; 2,2,2-trifluoroacetic acid | 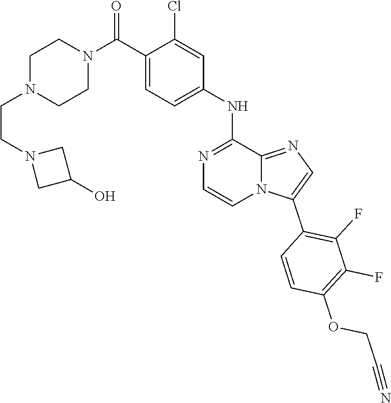 [SMILES: N#CCOc1ccc(-c2cnc3c(Nc4ccc(C(=O)N5CCN(CCN6CC(O)C6)CC5)c(Cl)c4)nccn23)c(F)c1F] | 623.3 |
| 97 | 2-[4-[8-[3-chloro-4-[4-[2-(3- methoxyazetidin-1- yl)ethyl]piperazine-1- carbonyl]anilino]imidazo[1,2- a]pyrazin-3-yl]-2,3- difluorophenoxy]acetonitrile; 2,2,2-trifluoroacetic acid | 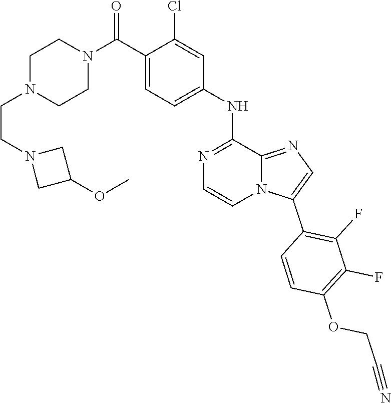 [SMILES: COC1CN(CCN2CCN(C(=O)c3ccc(Nc4nccn5c(-c6ccc(OCC#N)c(F)c6F)cnc45)cc3Cl)CC2)C1] | 637.2 |
| 98 | 2-[4-[8- [3-chloro-4-[4-[2-(3- methylsulfonylazetidin-1- yl)ethyl]piperazine-1- carbonyl]anilino]imidazo[1,2- a]pyrazin-3-yl]-2,3- difluorophenoxy]acetonitrile; 2,2,2-trifluoroacetic acid | 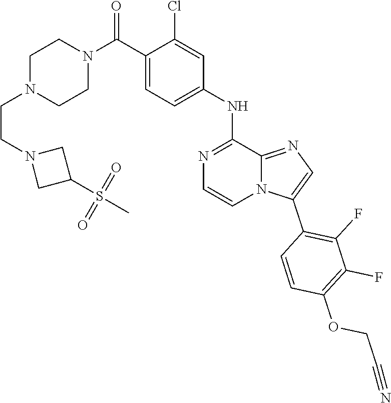 [SMILES: CS(=O)(=O)C1CN(CCN2CCN(C(=O)c3ccc(Nc4nccn5c(-c6ccc(OCC#N)c(F)c6F)cnc45)cc3Cl)CC2)C1] | 685.1 |
| 99 | 2-[4-[8-[3-chloro-4-[4-[2-[3- [(dimethylamino)methyl]azetidin- 1-yl]ethyl]piperazine-1- carbonyl]anilino]imidazo[1,2- a]pyrazin-3-yl]-2,3- difluorophenoxy]acetonitrile; 2,2,2-trifluoroacetic acid | 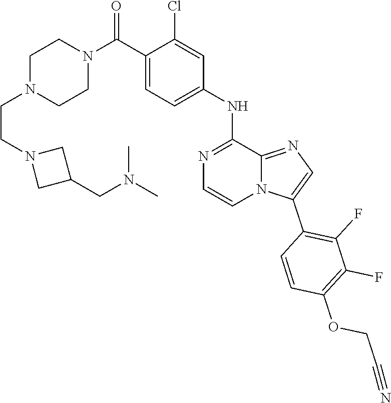 [SMILES: CN(C)CC1CN(CCN2CCN(C(=O)c3ccc(Nc4nccn5c(-c6ccc(OCC#N)c(F)c6F)cnc45)cc3Cl)CC2)C1] | 664.4 |
| 100 | 2-[4-[8-[3-chloro-4-[4-[2-[3- (dimethylamino)pyrrolidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 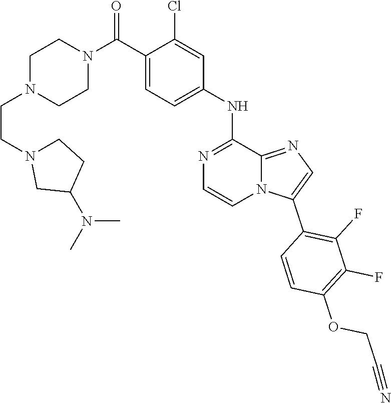 [SMILES: CN(C)C1CCN(CCN2CCN(C(=O)c3ccc(Nc4nccn5c(-c6ccc(OCC#N)c(F)c6F)cnc45)cc3Cl)CC2)C1] | 664.1 |
| 101 | 2-[4-[8-[3-chloro-4-[4-[2-[4- (dimethylamino)piperidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 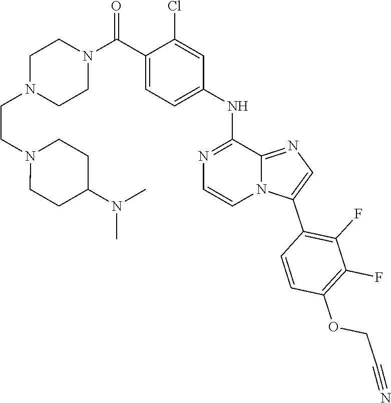 [SMILES: CN(C)C1CCN(CCN2CCN(C(=O)c3ccc(Nc4nccn5c(-c6ccc(OCC#N)c(F)c6F)cnc45)cc3Cl)CC2)CC1] | 678.1 |
| 102 | 2-[4-[8-[3-chloro-4-[4-[2-[3- (dimethylamino)azetidin-1- yl]ethyl]piperazine-1- carbonyl]anilino]imidazo[1,2- a]pyrazin-3-yl]-2,3- difluorophenoxy]acetonitrile; 2,2,2-trifluoroacetic acid | 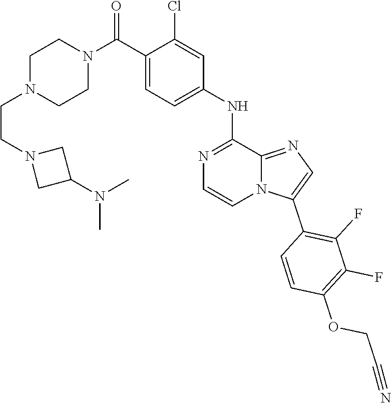 [SMILES: CN(C)C1CN(CCN2CCN(C(=O)c3ccc(Nc4nccn5c(-c6ccc(OCC#N)c(F)c6F)cnc45)cc3Cl)CC2)C1] | 650.3 |
| 103 | 2-[4-[8-[3-chloro-4-[4-[2-(3- methylsulfonylpyrrolidin-1- yl)ethyl]piperazine-1- carbonyl]anilino]imidazo[1,2- a]pyrazin-3-yl]-2,3- difluorophenoxy]acetonitrile; 2,2,2-trifluoroacetic acid | 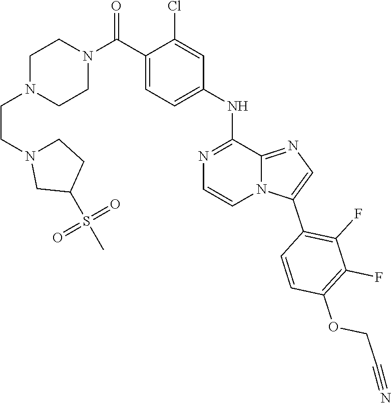 [SMILES: CS(=O)(=O)C1CCN(CCN2CCN(C(=O)c3ccc(Nc4nccn5c(-c6ccc(OCC#N)c(F)c6F)cnc45)cc3Cl)CC2)C1] | 699.1 |

The following additional Examples have been prepared with the methods described above:

| | | | ESI MS |
|---|---|---|---|
| Ex. | Name | Structure | [M + H]⁺ |
| 104 | (2-(4-(2-chloro-4-((3-(4- (cyanomethoxy)-2,3- difluorophenyl)imidazo[1,2- a]pyrazin-8- yl)amino)benzoyl)piperazin-1- yl)ethyl)proline trifluoroacetate | 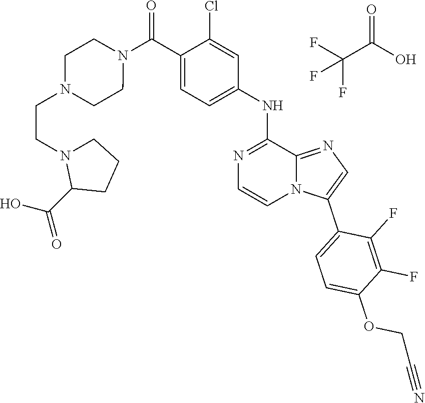 [SMILES: N#CCOc1ccc(-c2cnc3c(Nc4ccc(C(=O)N5CCN(CCN6CCCC6C(=O)O)CC5)c(Cl)c4)nccn23)c(F)c1F ; O=C(O)C(F)(F)F] | 665.2 |
| 105 | 2-(4-(8-((4-(4-(2-(3-(2H- tetrazol-5-yl)azetidin-1- yl)ethyl)piperazine-1- carbonyl)-3- chlorophenyl)amino)imidazo [1,2-a]pyrazin-3-yl)-2,3- difluorophenoxy)acetonitrile trifluoroacetate | 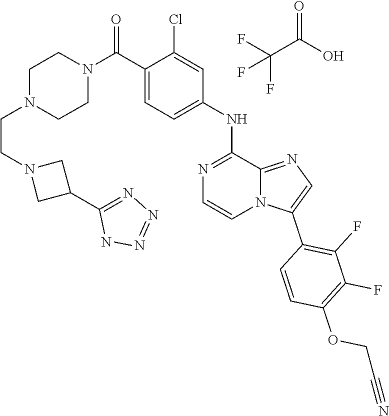 | 675.1 |
| 106 | 2-(1-(2-chloro-4-((3-(3-fluoro- 4- methoxyphenyl)imidazo[1,2- a]pyrazin-8- yl)amino)benzoyl)-4- hydroxypiperidin-4-yl)-N,N- dimethylacetamide | 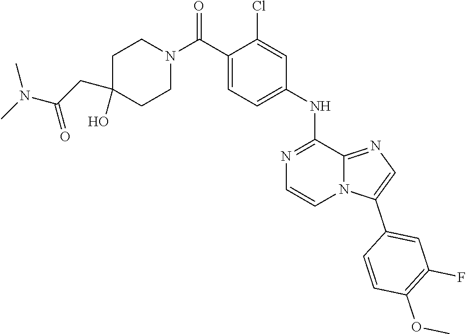 [SMILES: COc1ccc(-c2cnc3c(Nc4ccc(C(=O)N5CCC(O)(CC(=O)N(C)C)CC5)c(Cl)c4)nccn23)cc1F] | 581.2 |
| 107 | 1-(2-chloro-4-((3-(3-fluoro-4- methoxyphenyl)imidazo[1,2- a]pyrazin-8- yl)amino)benzoyl)-N- ((2S,3R,4R,5R)-2,3,4,5,6- pentahydroxyhexyl)piperidine- 4-carboxamide | 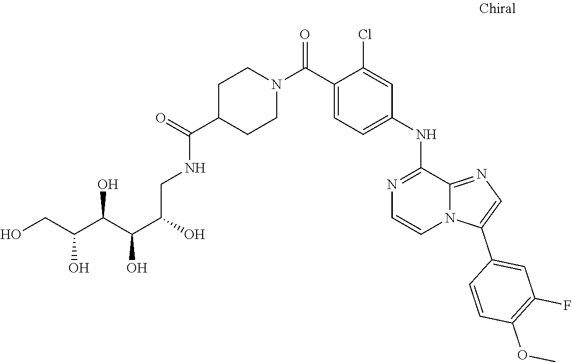 [SMILES: COc1ccc(-c2cnc3c(Nc4ccc(C(=O)N5CCC(C(=O)NC[C@H](O)[C@@H](O)[C@H](O)[C@H](O)CO)CC5)c(Cl)c4)nccn23)cc1F] | 687.3 |
| 108 | 1-(2-(4-(2-chloro-4-((3-(4- (cyanomethoxy)-2,3- difluorophenyl)imidazo[1,2- a]pyrazin-8- yl)amino)benzoyl)piperazin-1- yl)-2-oxoethyl)pyrrolidine-3- carboxylic acid trifluoroacetate | 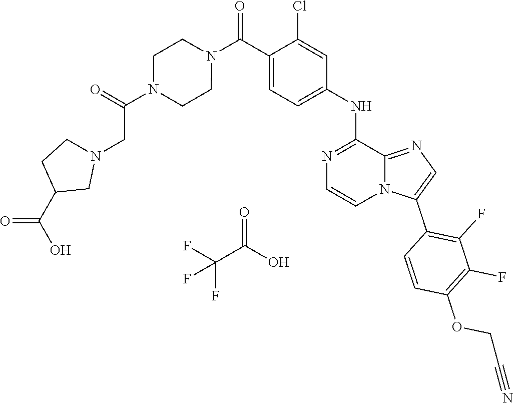 [SMILES: N#CCOc1ccc(-c2cnc3c(Nc4ccc(C(=O)N5CCN(C(=O)CN6CCC(C(=O)O)C6)CC5)c(Cl)c4)nccn23)c(F)c1F ; O=C(O)C(F)(F)F] | 679.5 |
| 109 | (4-(2-bromo-4-((3-(2,3- difluoro-4- methoxyphenyl)imidazo[1,2- a]pyrazin-8-yl)amino)-6- fluorobenzoyl)piperazin-1- yl)((2S,4R)-4- hydroxypyrrolidin-2- yl)methanone dihydrochloride | 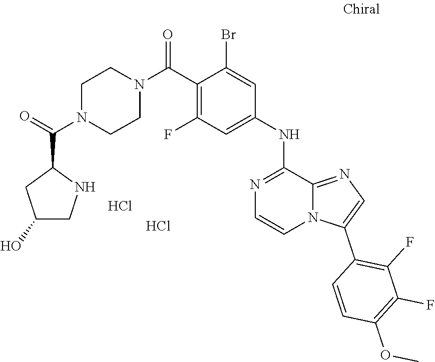 [SMILES: COc1ccc(-c2cnc3c(Nc4cc(F)c(C(=O)N5CCN(C(=O)[C@@H]6C[C@@H](O)CN6)CC5)c(Br)c4)nccn23)c(F)c1F ; Cl ; Cl] | 672.4, 674.4 (M − H)- |
| 110 | (2-chloro-4-((3-(3-fluoro-4- methoxyphenyl)imidazo[1,2- a]pyrazin-8- yl)amino)phenyl)(4-hydroxy- 4- ((methylamino)methyl)piperidin- 1-yl)methanone | 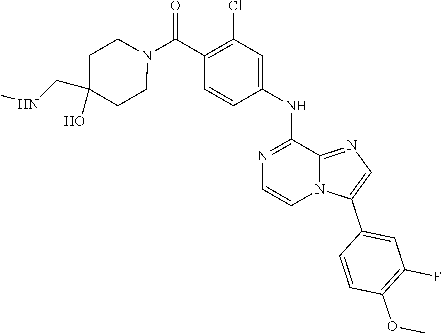 [SMILES: CNCC1(O)CCN(C(=O)c2ccc(Nc3nccn4c(-c5ccc(OC)c(F)c5)cnc34)cc2Cl)CC1] | 539.2 |
| 111 | (2-chloro-4-((3-(2,3-difluoro- 4- methoxyphenyl)imidazo[1,2- a]pyrazin-8- yl)amino)phenyl)(4-hydroxy- 4- ((methylamino)methyl)piperidin- 1-yl)methanone | 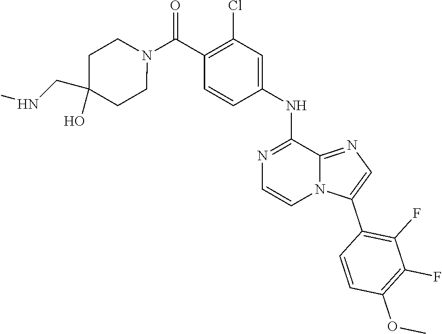 [SMILES: CNCC1(O)CCN(C(=O)c2ccc(Nc3nccn4c(-c5ccc(OC)c(F)c5F)cnc34)cc2Cl)CC1] | 555.5 (M − H)- |
| 112 | [2-chloro-4-[[3-[2,3-difluoro- 4- (fluoromethoxy)phenyl]imidazo [1,2-a]pyrazin-8- yl]amino]phenyl]-[4-[(2S,4R)- 4-hydroxypyrrolidine-2- carbonyl]piperazin-1- yl]methanone trifluoroacetate | 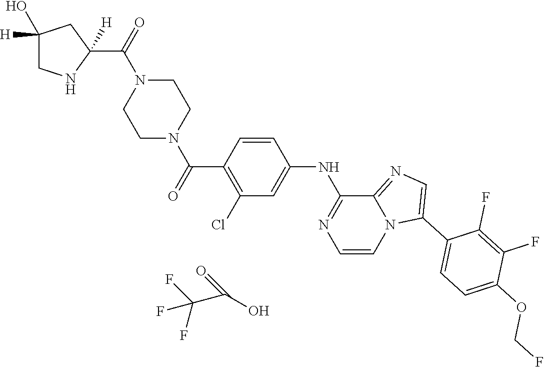 [SMILES: O=C(c1ccc(Nc2nccn3c(-c4ccc(OCF)c(F)c4F)cnc23)cc1Cl)N1CCN(C(=O)[C@@H]2C[C@@H](O)CN2)CC1 ; O=C(O)C(F)(F)F] | 630.1 |
| 113 | [2-chloro-4-[[3-[4- (difluoromethoxy)-2,3- difluoro-phenyl]imidazo[1,2- a]pyrazin-8-yl]amino]phenyl]- [4-[(2S,4R)-4- hydroxypyrrolidine-2- carbonyl]piperazin-1- yl]methanone formate | 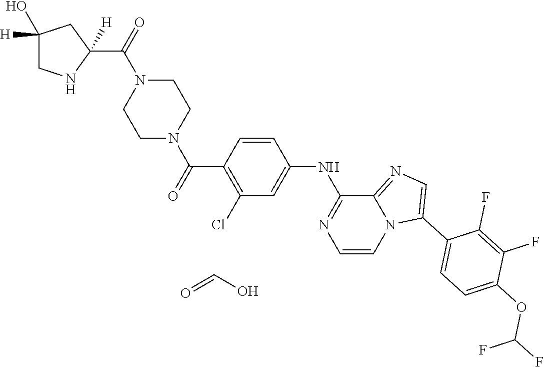 [SMILES: O=C(c1ccc(Nc2nccn3c(-c4ccc(OC(F)F)c(F)c4F)cnc23)cc1Cl)N1CCN(C(=O)[C@@H]2C[C@@H](O)CN2)CC1 ; O=CO] | 648.2 |
| 114 | (4-(4-((3-(4- (difluoromethoxy)-2,3- difluorophenyl)imidazo[1,2- a]pyrazin-8-yl)amino)-2- fluorobenzoyl)piperazin-1- yl)(1-(pyrrolidin-3- ylmethyl)piperidin-4- yl)methanone bis(2,2,2- trifluoroacetate) | 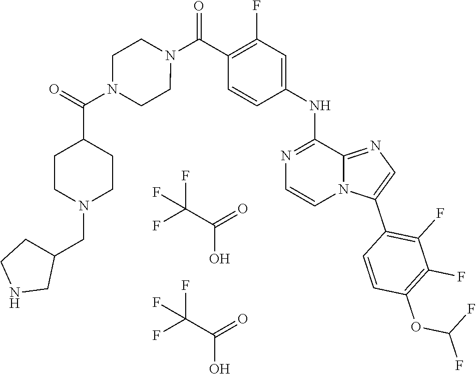 | 713.3 |

### Assay Procedures

### Antimicrobial Susceptibility Testing:

### 90% Growth Inhibitory Concentration (IC90) Determination

The in vitro antimicrobial activity of the compounds was determined according to the following procedure:

The assay used a 10-points Iso-Sensitest broth medium to measure quantitatively the in vitro activity of the compounds against Acinetobacter baumannii ATCC17978 and Acinetobacter baumannii ATCC17961.

Stock compounds in DMSO were serially twofold diluted (e.g. range from 50 to 0.097 μM final concentration) in 384 wells microtiter plates and inoculated with 49 μl the bacterial suspension in Iso-Sensitest medium to have a final cell concentration of ˜5×10⁽⁵⁾ CFU/ml in a final volume/well of 50 ul/well. Microtiter plates were incubated at 35±2° C.

Bacterial cell growth was determined with the measurement of optical density at λ=600 nm each 20 minutes over a time course of 16 h. Growth inhibition was calculated during the logarithmic growth of the bacterial cells with determination of the concentration inhibiting 50% (IC50) and 90% (IC90) of the growth.

Table 1 provides the 90% growth inhibitory concentrations (IC90) in micromoles per liter of the compounds of present invention obtained against the strain Acinetobacter baumannii ATCC17978 and Acinetobacter baumannii ATCC17961.

Particular compounds of the present invention exhibit an IC90 (A. baumannii ATCC17978 and Acinetobacter baumannii ATCC17961)≤25 μmol/l.

More particular compounds of the present invention exhibit an IC90 (A. baumannii ATCC17978 and Acinetobacter baumannii ATCC17961)≤5 μmol/l.

Most particular compounds of the present invention exhibit an IC90 (A. baumannii ATCC17978 and Acinetobacter baumannii ATCC17961)≤1 μmol/l.

| | ATCC17978 |
|---|---|
| Example | IC90 [μM] |
| 1 | 1.9 |
| 2 | 0.47 |
| 3 | 0.12 |
| 4 | 0.12 |
| 5 | 0.14 |
| 6 | 0.11 |
| 7 | 0.1 |
| 8 | 0.17 |
| 9 | 1.6 |
| 10 | 0.064 |
| 11 | 0.14 |
| 12 | 0.049 |
| 13 | 0.28 |
| 14 | 0.028 |
| 15 | 0.11 |
| 16 | 0.1 |
| 17 | 0.22 |
| 18 | 0.18 |
| 19 | 0.3 |
| 20 | 0.13 |
| 21 | 0.87 |
| 22 | 0.69 |
| 23 | 0.16 |
| 24 | 0.15 |
| 25 | 0.079 |
| 26 | 0.086 |
| 27 | 1.5 |
| 28 | 0.14 |
| 29 | 0.37 |
| 30 | 0.19 |
| 31 | 0.32 |
| 32 | 0.3 |
| 34 | 0.031 |
| 36 | 0.041 |
| 37 | 0.031 |
| 38 | 0.57 |
| 39 | 0.2 |
| 40 | 0.15 |
| 41 | 0.13 |
| 42 | 0.2 |
| 43 | 0.14 |
| 44 | 0.3 |
| 45 | 0.88 |
| 46 | 0.89 |
| 47 | 0.43 |
| 48 | 0.041 |
| 51 | 0.23 |
| 52 | 0.088 |
| 53 | 0.056 |
| 54 | 0.13 |
| 55 | 0.14 |
| 56 | 0.24 |
| 57 | 0.33 |
| 58 | 0.33 |
| 59 | 1.2 |
| 60 | 0.14 |
| 61 | 0.58 |
| 62 | 0.9 |
| 63 | 1.1 |
| 64 | 1.4 |
| 65 | 1.5 |
| 66 | 5.9 |
| 67 | 0.17 |
| 68 | 0.11 |
| 69 | 0.14 |
| 70 | 0.27 |
| 71 | 0.28 |
| 72 | 0.51 |
| 73 | 0.53 |
| 74 | 2.7 |
| 75 | 0.085 |
| 76 | 0.24 |
| 77 | 0.073 |
| 78 | 0.28 |
| 79 | 0.16 |
| 80 | 0.087 |
| 81 | 0.29 |
| 82 | 1 |
| 83 | 0.14 |
| 84 | 0.11 |
| 85 | 0.046 |
| 86 | 0.049 |
| 87 | 0.052 |
| 88 | 0.09 |
| 89 | 0.042 |
| 90 | 0.043 |
| 91 | 0.049 |
| 92 | 0.05 |
| 93 | 0.053 |
| 94 | 0.054 |
| 95 | 0.056 |
| 96 | 0.073 |
| 97 | 0.1 |
| 98 | 0.14 |
| 99 | 0.17 |
| 100 | 0.17 |
| 101 | 0.17 |
| 102 | 0.17 |
| 103 | 0.18 |

| | ATCC17961 |
|---|---|
| Example | IC90 [μM] |
| 33 | 0.03392 |
| 35 | 0.0412 |
| 49 | 0.07219 |
| 50 | 0.0821 |

| | ATCC17978 | ATCC17961 |
|---|---|---|
| Example | IC90 [μM] | IC90 [μM] |
| 104 | 0.63 | |
| 105 | 0.36 | |
| 106 | 0.54 | |
| 107 | 0.26 | |
| 108 | | 0.35 |
| 109 | | 0.069 |
| 110 | | 0.080 |
| 111 | | 0.068 |
| 112 | | 0.022 |
| 113 | | 0.022 |
| 114 | | 0.056 |

### Example 115

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 116

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example 117

A compound of formula (I) can be used in a manner known per se as the active ingredient for the production of an infusion solution of the following composition:

| Active ingredient | 100 mg |
|---|---|
| Lactic acid 90% | 100 mg |
| NaOH q.s. or HCl q.s. for adjustment to pH | 4.0 |

Sodium chloride q.s. or glucose q.s. for adjustment of the osmolality
to 290 mOsm/kg

| | |
|---|---|
| Water for injection (WFI) ad | 100 ml |

### Example 118

A compound of formula (I) can be used in a manner known per se as the active ingredient for the production of an infusion solution of the following composition:

| | | |
|---|---|---|
| Active ingredient | 100 | mg |
| Hydroxypropyl-beta-cyclodextrin | 10 | g |

| | |
|---|---|
| NaOH q.s. or HCl q.s. for adjustment to pH | 7.4 |

Sodium chloride q.s. or glucose q.s. for adjustment of the osmolality
to 290 mOsm/kg

| | | |
|---|---|---|
| Water for injection (WFI) ad | 100 | ml |

1. A compound of formula (I): 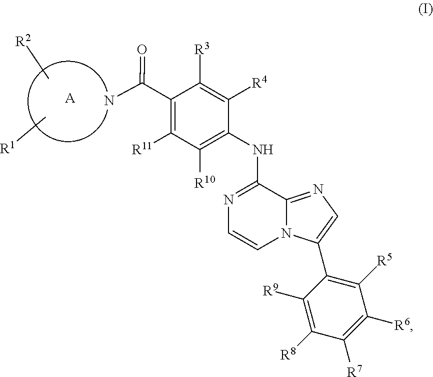
or a pharmaceutically acceptable salt thereof, wherein:
A is a mono- or bicyclic C₂-C₉-heterocycloalkyl ring;
R¹ is hydrogen, amino, C₁-C₆-alkyl-NH—, (C₁-C₆-alkyl)₂N—, sulfamoyl, C₁-C₆-alkylsulfamoyl, di-C₁-C₆-alkylsulfamoyl, C₁-C₆-alkylsulfonyl-NH—C(O)—, C₁-C₆-alkylsulfonyl-N(C₁-C₆-alkyl)-C(O)—, hydroxy, carboxy, carbamimidoyl, carbamoyl, C₁-C₆-alkoxycarbonyl, C₁-C₆-alkoxycarbonyl-NH—, C₁-C₆-alkoxycarbonyl-N(C₁-C₆-alkyl)-, carboxy-NH—, carboxy-N(C₁-C₆-alkyl)-, a group 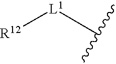
or a group 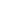
R² is hydrogen, hydroxy, carbamoyl, C₁-C₆-alkyl-NH—C(O)— or (C₁-C₆-alkyl)₂N—C(O)—;
R³ is hydrogen, halogen, NO₂ or CN;
R⁵, R⁶, R⁷, R⁸ and R⁹ are each independently hydrogen, halogen, C₁-C₆-alkoxycarbonyl-C₁-C₆-alkoxy, amino, C₁-C₆-alkyl-NH—, (C₁-C₆-alkyl)₂N—, hydroxy, C₁-C₆-alkoxy, C₁-C₆-alkylsulfanyl, C₁-C₆-alkylsulfonyloxy, C₃-C₁₂-cycloalkyl-C₁-C₆-alkoxy, halo-C₁-C₆-alkoxy, C₆-C₁₄-aryl-C₁-C₆-alkoxy, C₁-C₁₃-heteroaryloxy, C₁-C₁₃-heteroaryl-C₁-C₆-alkoxy, cyano-C₁-C₆-alkoxy, C₃-C₁₂-cycloalkyloxy, C₂-C₆-alkynyloxy, C₁-C₆-alkoxy-C₂-C₆-alkynyloxy, cyano-C₃-C₁₂-cycloalkyloxy, cyano-C₃-C₁₂-cycloalkyl-C₁-C₆-alkoxy, amino-C₂-C₆-alkynyloxy, hydroxy-C₂-C₆-alkynyloxy, halo-C₁-C₆-alkyl, sulfamoyl, C₁-C₆-alkylsulfamoyl, C₁-C₆-alkyl, amino-C₁-C₆-alkoxy-C₂-C₆-alkynyloxy or amino-C₁-C₆-alkoxy;
R⁴, R¹⁰ and R¹¹ are each independently hydrogen, halogen or C₁-C₆-alkyl;
R¹² is C₁-C₆-alkyl substituted with R¹⁷, R¹⁸, R¹⁹, R²⁰ or R²¹, or a combination thereof,
R¹³, R¹⁴, R¹⁵ and R¹⁶ are each independently hydrogen, halogen, cyano, hydroxy, C₁-C₆-alkylsulfonyl, amino, HO—SO₂—, C₁-C₆-alkyl-NH—, (C₁-C₆-alkyl)₂N—, C₁-C₆-alkyl, C₁-C₆-alkoxy, amino-C₁-C₆-alkyl, C₁-C₆-alkyl-NH—C₁-C₆-alkyl-, (C₁-C₆-alkyl)₂N—C₁-C₆-alkyl-, hydroxy-C₁-C₆-alkyl, C₃-C₁₂-cycloalkyl, C₁-C₁₃-heteroaryl, C₁-C₆-alkyl-C₁-C₁₃-heteroaryl, C₂-C₉-heterocycloalkyl-C₁-C₆-alkyl-, carbamoyl, C₁-C₆-alkyl-NH—C(O)—, (C₁-C₆-alkyl)₂N—C(O)— or carboxy;
R¹⁷, R¹⁸, R¹⁹, R²⁰ and R²¹ are each independently hydrogen, HO—SO₂—, hydroxy, cyano, amino, C₁-C₆-alkyl-NH—, (C₁-C₆-alkyl)₂N—, cyano-C₁-C₆-alkyl-NH—, cyano-C₁-C₆-alkyl-N(C₁-C₆-alkyl)-, amino-C₁-C₆-alkyl-C(O)—NH—, C₁-C₆-alkyl-NH—C₁-C₆-alkyl-C(O)—NH—, (C₁-C₆-alkyl)₂N—C₁-C₆-alkyl-C(O)—NH—, amino-C₁-C₆-alkyl-C(O)—N(C₁-C₆-alkyl)-, C₁-C₆-alkyl-NH—C₁-C₆-alkyl-C(O)—N(C₁-C₆-alkyl)-, (C₁-C₆-alkyl)₂N—C₁-C₆-alkyl-C(O)—N(C₁-C₆-alkyl)-, hydroxy-C₁-C₆-alkyl-NH—, hydroxy-C₁-C₆-alkyl-C(O)—NH—, hydroxy-C₁-C₆-alkyl-C(O)—N(C₁-C₆-alkyl)-, guanidino, carboxy, C₁-C₆-alkoxycarbonyl, C₁-C₆-alkoxycarbonyl-NH—, carbamoyl, C₁-C₆-alkyl-NH—C(O)—, (C₁-C₆-alkyl)₂N—C(O)—, C₁-C₆-alkyl-C(O)—NH—, C₁-C₆-alkyl-C(O)—N(C₁-C₆-alkyl)-, hydroxy-C₁-C₆-alkoxy, C₁-C₆-alkoxy, amino-C₁-C₆-alkyl-CH(NH₂)—C(O)—NH—, carboxy-C₁-C₆-alkyl-CH(NH₂)—C(O)—NH—, carboxy-CH(NH₂)—C₁-C₆-alkyl-C(O)—NH—, amino-C₁-C₆-alkyl-CH(COOH)—NH—, carboxy-C₁-C₆-alkyl-N(C₁-C₆-alkyl)-, carboxy-C₁-C₆-alkyl-NH—, ureido, amino-C₁-C₆-alkyl, C₁-C₆-alkyl-NH—C₁-C₆-alkyl- or (C₁-C₆-alkyl)₂N—C₁-C₆-alkyl-;
L¹ is a covalent bond, carbonyl, —NH—, —N(C₁-C₆-alkyl)-, —NH—C(O)—, —C(O)—NH—, —C(O)—N(C₁-C₆-alkyl)- or —N(C₁-C₆-alkyl)-C(O)—;
L² is a covalent bond, —C₁-C₆-alkyl-, carbonyl, SO₂, —C(O)—C₁-C₆-alkyl-, —C₁-C₆-alkyl-C(O)—, —C₁-C₆-alkyl-NH—C(O)—, —C₁-C₆-alkyl-N(C₁-C₆-alkyl)-C(O)—, —C₁-C₆-alkyl-O—C(O)—, —NH—C(O)—, —CH(NH₂)—C(O)—, —O—, —NH—C₁-C₆-alkyl-, —N(C₁-C₆-alkyl)-C₁-C₆-alkyl-, —C(O)—NH—C₁-C₆-alkyl-, —C(O)—N(C₁-C₆-alkyl)-C₁-C₆-alkyl-, —C₁-C₆-alkyl-CH(NH₂)—C(O)—, or —C(O)—NH—; and
B is C₆-C₁₄-aryl, C₁-C₁₃-heteroaryl, C₃-C₁₂-cycloalkyl, or C₂-C₉-heterocycloalkyl.

2. The compound of formula (I) according to claim 1, or a pharmaceutically acceptable salt thereof, wherein R¹ is hydrogen, (C₁-C₆-alkyl)₂N—, hydroxy, carboxy, a group 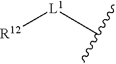
or a group 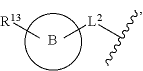
wherein:
R¹² is C₁-C₆-alkyl substituted with R¹⁷, R¹⁸, R¹⁹, R²⁰ or R²¹, or a combination thereof,
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl, C₁-C₆-alkylsulfonyl, C₁-C₁₃-heteroaryl or C₂-C₉-heterocycloalkyl-C₁-C₆-alkyl-;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen, hydroxy or amino;
R¹⁹, R²⁰ and R²¹ are each independently hydrogen or hydroxy;
L¹ is a covalent bond, carbonyl, —N(C₁-C₆-alkyl)-, —NH—C(O)— or —N(C₁-C₆-alkyl)-C(O)—;
L² is a covalent bond, —C₁-C₆-alkyl-, carbonyl, —C₁-C₆-alkyl-C(O)—, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-; and
B is C₁-C₁₃-heteroaryl or C₂-C₉-heterocycloalkyl.

3. The compound of formula (I) according to claim 1, or a pharmaceutically acceptable salt thereof, wherein R¹ is a group 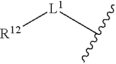
or a group 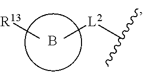
wherein:
R¹² is C₁-C₆-alkyl substituted with R¹⁷;
R¹³ is hydrogen or hydroxy;
R¹⁷ is C₁-C₆-alkyl-NH— or (C₁-C₆-alkyl)₂N—;
L¹ is a covalent bond, carbonyl or —NH—C(O)—;
L² is carbonyl or —C₁-C₆-alkyl-NH—C(O)—; and
B is C₂-C₉-heterocycloalkyl.

4. The compound of formula (I) according to claim 1, or a pharmaceutically acceptable salt thereof, wherein R¹ is a group 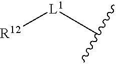
or a group 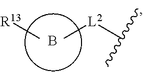
wherein:
R¹² is C₁-C₂-alkyl substituted with R¹⁷;
R¹³ is hydrogen or hydroxy;
R¹⁷ is methylamino or dimethylamino;
L¹ is a covalent bond, carbonyl or —NH—C(O)—;
L² is carbonyl or —CH₂NH—C(O)—; and
B is piperazinyl, pyrrolidinyl or azetidinyl.

5. The compound of formula (I) according to claim 1, or a pharmaceutically acceptable salt thereof, wherein R² is hydrogen or hydroxy.

6. The compound of formula (I) according to claim 1, or a pharmaceutically acceptable salt thereof, wherein R³ is hydrogen, halogen or CN.

7.-8. (canceled)

9. The compound of formula (I) according to claim 1, or a pharmaceutically acceptable salt thereof, wherein R⁴ is hydrogen.

10. The compound of formula (I) according to claim 1, or a pharmaceutically acceptable salt thereof, wherein R⁵ is hydrogen or halogen.

11. (canceled)

12. The compound of formula (I) according to claim 1, or a pharmaceutically acceptable salt thereof, wherein R⁶ is hydrogen or halogen.

13. (canceled)

14. The compound of formula (I) according to claim 1, or a pharmaceutically acceptable salt thereof, wherein R⁷ is C₁-C₆-alkoxy, halo-C₁-C₆-alkoxy, cyano-C₁-C₆-alkoxy or hydroxy-C₂-C₆-alkynyloxy.

15. (canceled)

16. The compound of formula (I) according to claim 14, or a pharmaceutically acceptable salt thereof, wherein R⁷ is methoxy or cyanomethoxy.

17. The compound of formula (I) according to claim 1, or a pharmaceutically acceptable salt thereof, wherein R⁸ is hydrogen or halogen.

18. (canceled)

19. The compound of formula (I) according to claim 1, or a pharmaceutically acceptable salt thereof, wherein R⁹ is hydrogen.

20. The compound of formula (I) according to claim 1, or a pharmaceutically acceptable salt thereof, wherein R¹⁰ is hydrogen.

21. The compound of formula (I) according to claim 1, or a pharmaceutically acceptable salt thereof, wherein R¹¹ is hydrogen or halogen.

22. (canceled)

23. The compound of formula (I) according to claim 1, or a pharmaceutically acceptable salt thereof, wherein A is a monocyclic C₂-C₉-heterocycloalkyl ring.

24. The compound of formula (I) according to claim 23, or a pharmaceutically acceptable salt thereof, wherein A is piperazin-1-yl or 1-piperidyl.

25. The compound of formula (I) according claim 1, or a pharmaceutically acceptable salt thereof, wherein:
A is a monocyclic C₂-C₉-heterocycloalkyl ring;
R¹ is hydrogen, (C₁-C₆-alkyl)₂N—, hydroxy, carboxy, a group 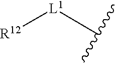
or a group 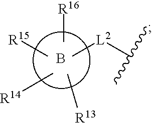
R² is hydrogen or hydroxy;
R³ is hydrogen, halogen or CN;
R⁵, R⁶ and R⁸ are each independently hydrogen or halogen;
R⁷ is C₁-C₆-alkoxy, halo-C₁-C₆-alkoxy, cyano-C₁-C₆-alkoxy or hydroxy-C₂-C₆-alkynyloxy;
R¹¹ is hydrogen or halogen;
R¹² is C₁-C₆-alkyl substituted with R¹⁷, R¹⁸, R¹⁹, R²⁰ or R²¹, or a combination thereof;
R⁴, R⁹, R¹⁰, R¹⁴, R¹⁵ and R¹⁶ are all hydrogen;
R¹³ is hydrogen, hydroxy, C₁-C₆-alkyl, C₁-C₆-alkoxy, amino-C₁-C₆-alkyl, hydroxy-C₁-C₆-alkyl, carboxy, carbamoyl, C₁-C₆-alkylsulfonyl, C₁-C₁₃-heteroaryl or C₂-C₉-heterocycloalkyl-C₁-C₆-alkyl-;
R¹⁷ is hydrogen, amino, C₁-C₆-alkyl-NH—, (C₁-C₆-alkyl)₂N—, hydroxy, hydroxy-C₁-C₆-alkyl-NH— or (C₁-C₆-alkyl)₂N—C₁-C₆-alkyl-;
R¹⁸ is hydrogen, hydroxy or amino;
R¹⁹, R²⁰ and R²¹ are each independently hydrogen or hydroxy;
B is C₁-C₁₃-heteroaryl or C₂-C₉-heterocycloalkyl;
L¹ is a covalent bond, carbonyl, —N(C₁-C₆-alkyl)-, —NH—C(O)— or —N(C₁-C₆-alkyl)-; and
L² is a covalent bond, —C₁-C₆-alkyl-, carbonyl, —C₁-C₆-alkyl-C(O)—, —C₁-C₆-alkyl-NH—C(O)—, —C₁-C₆-alkyl-O—C(O)—, —NH—C(O)—, —NH—C₁-C₆-alkyl- or —C(O)—NH—C₁-C₆-alkyl-.

26. The compound of formula (I) according to claim 1, or a pharmaceutically acceptable salt thereof, wherein:
A is a monocyclic C₂-C₉-heterocycloalkyl ring;
R¹ is a group 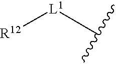
or a group 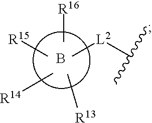
R² is hydrogen or hydroxy;
R³ is halogen;
R⁵, R⁶ and R⁸ are each independently hydrogen or halogen;
R⁷ is C₁-C₆-alkoxy or cyano-C₁-C₆-alkoxy;
R¹² is C₁-C₆-alkyl substituted with R¹⁷, R¹⁸, R¹⁹, R²⁰ or R²¹, or a combination thereof,
R⁴, R⁹, R¹⁰, R¹¹, R¹⁴, R¹⁵, R¹⁶, R¹⁸, R¹⁹, R²⁰ and R²¹ are all hydrogen;
R¹³ is hydrogen or hydroxy;
R¹⁷ is C₁-C₆-alkyl-NH— or (C₁-C₆-alkyl)₂N—;
B is C₂-C₉-heterocycloalkyl;
L¹ is a covalent bond, carbonyl or —NH—C(O)—; and
L² is a carbonyl or —C₁-C₆-alkyl-NH—C(O)—.

27. The compound of formula (I) according to claim 1, or a pharmaceutically acceptable salt thereof, wherein:
A is piperazinyl or piperidyl;
R¹ is a group 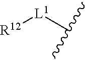
or a group 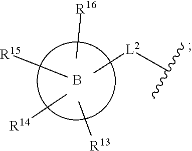
R² is hydrogen or hydroxy;
R³ is chloro;
R⁵ is hydrogen, fluoro or chloro;
R⁶ and R⁸ are each independently hydrogen or fluoro;
R⁷ is methoxy or cyanomethoxy;
R¹² is C₁-C₂-alkyl substituted with R¹⁷, R¹⁸, R¹⁹, R²⁰ or R²¹, or a combination thereof,
R⁴, R⁹, R¹⁰, R¹¹, R¹⁴, R¹⁵, R¹⁶, R¹⁸, R¹⁹, R²⁰ and R²¹ are all hydrogen;
R¹³ is hydrogen or hydroxy;
R¹⁷ is methylamino or dimethylamino;
B is piperazinyl, pyrrolidinyl or azetidinyl;
L¹ is a covalent bond, carbonyl or —NH—C(O)—; and
L² is carbonyl or —CH₂NH—C(O)—.

28. The compound of formula (I) according to claim 1, or a pharmaceutically acceptable salt thereof, wherein the compound of formula (I) is selected from:
[4-(2-aminoethyl)-1-piperidyl]-[2-chloro-4-[[3-(2,3-difluoro-4-methoxy-phenyl)imidazo[1,2-a]pyrazin-8-yl]amino]phenyl]methanone;
[2-chloro-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
[4-(aminomethyl)-1-piperidyl]-[2-chloro-4-[[3-(3-fluoro-4-methoxy-phenyl)imidazo[1,2-a]pyrazin-8-yl]amino]phenyl]methanone;
[4-(aminomethyl)-1-piperidyl]-[2-bromo-4-[[3-[4-(difluoromethoxy)phenyl]imidazo[1,2-a]pyrazin-8-yl]amino]phenyl]methanone;
[4-(aminomethyl)-1-piperidyl]-[2-chloro-4-[[3-(2,3-difluoro-4-methoxy-phenyl)imidazo[1,2-a]pyrazin-8-yl]amino]phenyl]methanone;
[4-(aminomethyl)-1-piperidyl]-[4-[[3-[4-(difluoromethoxy)phenyl]imidazo[1,2-a]pyrazin-8-yl]amino]-2-iodo-phenyl]methanone;
[4-[[3-(3-chloro-4-methoxy-phenyl)imidazo[1,2-a]pyrazin-8-yl]amino]phenyl]-morpholino-methanone;
[4-[[3-(4-methoxyphenyl)imidazo[1,2-a]pyrazin-8-yl]amino]phenyl]-morpholino-methanone;
[4-[[3-(3-chloro-4-methoxy-phenyl)imidazo[1,2-a]pyrazin-8-yl]amino]phenyl]-(4-hydroxy-1-piperidyl)methanone;
[4-(2-hydroxyethyl)piperazin-1-yl]-[4-[[3-(4-methoxyphenyl)imidazo[1,2-a]pyrazin-8-yl]amino]phenyl]methanone;
[4-[[3-(4-methoxyphenyl)imidazo[1,2-a]pyrazin-8-yl]amino]phenyl]-(4-methylpiperazin-1-yl)methanone;
2-[4-(aminomethyl)piperidine-1-carbonyl]-5-[[3-[4-(difluoromethoxy)phenyl]imidazo[1,2-a]pyrazin-8-yl]amino]benzonitrile;
[4-[[3-(3-chloro-4-methoxy-phenyl)imidazo[1,2-a]pyrazin-8-yl]amino]phenyl]-(1-piperidyl)methanone;
aziridin-1-yl-[2-chloro-4-[[3-(2,3-difluoro-4-methoxy-phenyl)imidazo[1,2-a]pyrazin-8-yl]amino]phenyl]methanone;
4-[2-chloro-4-[[3-(2,3-difluoro-4-methoxy-phenyl)imidazo[1,2-a]pyrazin-8-yl]amino]benzoyl]piperazine-2-carboxylic acid;
[4-[[3-(3-chloro-4-methoxy-phenyl)imidazo[1,2-a]pyrazin-8-yl]amino]phenyl]-(4-methylpiperazin-1-yl)methanone;
[3-(dimethylamino)pyrrolidin-1-yl]-[4-[[3-(4-methoxyphenyl)imidazo[1,2-a]pyrazin-8-yl]amino]phenyl]methanone;
2-[4-[8-[4-[4-(aminomethyl)piperidine-1-carbonyl]-3-chloro-anilino]imidazo[1,2-a]pyrazin-3-yl]-2,3-difluoro-phenoxy]acetonitrile;
2-[4-[8-[3-chloro-4-[4-[2-(dimethylamino)ethyl]piperazine-1-carbonyl]anilino]imidazo[1,2-a]pyrazin-3-yl]-2,3-difluoro-phenoxy]acetonitrile;
1-[2-chloro-4-[[3-(3-fluoro-4-methoxy-phenyl)imidazo[1,2-a]pyrazin-8-yl]amino]benzoyl]-N-[2-(methylamino)ethyl]piperidine-4-carboxamide;
[4-(aminomethyl)-1-piperidyl]-[2-bromo-4-[[3-(3-fluoro-4-methoxy-phenyl)imidazo[1,2-a]pyrazin-8-yl]amino]phenyl]methanone;
[4-[2-(aminomethyl)morpholine-4-carbonyl]-1-piperidyl]-[2-chloro-4-[[3-(3-fluoro-4-methoxy-phenyl)imidazo[1,2-a]pyrazin-8-yl]amino]phenyl]methanone;
2-[3-chloro-4-[8-[3-chloro-4-[4-[(dimethylamino)methyl]piperidine-1-carbonyl]anilino]imidazo[1,2-a]pyrazin-3-yl]-2-fluoro-phenoxy]acetonitrile;
1-[4-[2-chloro-4-[[3-(3-fluoro-4-methoxy-phenyl)imidazo[1,2-a]pyrazin-8-yl]amino]benzoyl]piperazin-1-yl]-2-(methylamino)ethanone;
[2-chloro-4-[[3-[4-(difluoromethoxy)phenyl]imidazo[1,2-a]pyrazin-8-yl]amino]phenyl]-(4-piperazin-1-yl-1-piperidyl)methanone;
2-[5-chloro-4-[8-[3-chloro-4-[4-[3-(hydroxymethyl)piperazine-1-carbonyl]piperidine-1-carbonyl]anilino]imidazo[1,2-a]pyrazin-3-yl]-2-fluoro-phenoxy]acetonitrile;
[2-chloro-4-[[3-(3-fluoro-4-methoxy-phenyl)imidazo[1,2-a]pyrazin-8-yl]amino]phenyl]-[4-[2-(methylamino)ethyl]piperazin-1-yl]methanone;
piperazin-2-ylmethyl 1-[2-chloro-4-[[3-[4-(cyanomethoxy)-2,3-difluoro-phenyl]imidazo[1,2-a]pyrazin-8-yl]amino]benzoyl]piperidine-4-carboxylate;
[2-chloro-4-[[3-(3-fluoro-4-methoxy-phenyl)imidazo[1,2-a]pyrazin-8-yl]amino]phenyl]-[4-[2-(2-hydroxyethylamino)ethyl]piperazin-1-yl]methanone;
2-[4-[8-[3-chloro-4-[4-[2-(hydroxymethyl)piperazine-1-carbonyl]piperidine-1-carbonyl]anilino]imidazo[1,2-a]pyrazin-3-yl]-2,3-difluoro-phenoxy]acetonitrile;
1-[2-chloro-4-[[3-(2,3-difluoro-4-methoxy-phenyl)imidazo[1,2-a]pyrazin-8-yl]amino]benzoyl]-N-[(3R)-pyrrolidin-3-yl]piperidine-4-carboxamide;
1-[2-chloro-4-[[3-(3-fluoro-4-methoxy-phenyl)imidazo[1,2-a]pyrazin-8-yl]amino]benzoyl]-N-[(3R)-pyrrolidin-3-yl]piperidine-4-carboxamide;
2-[4-[8-[3-chloro-4-[4-(1H-tetrazol-5-yl)piperidine-1-carbonyl]anilino]imidazo[1,2-a ]pyrazin-3-yl]-2,3-difluoro-phenoxy]acetonitrile;
2- [4-[8-[3-bromo-4-[4-[2-(dimethylamino)ethyl]piperazine-1-carbonyl ]anilino]imidazo[l,2-a]pyrazin-3-yl]-2,3-difluoro-phenoxy]acetonitrile;
N-(azetidin-3-ylmethyl)-1-[2-chloro-4-[[3-(3-fluoro-4-methoxy-phenyl)imidazo[l,2-a ]pyrazin-8-yl]amino]benzoyl]piperidine-4-carboxamide;
1-[2-bromo-4-[[3-(3-fluoro-4-methoxy-phenyl)imidazo[1,2-a]pyrazin-8-yl ]amino]benzoyl]-N-[2-(methylamino)ethyl]piperidine-4-carboxamide;
4-[1-[2-chloro-4-[[3-(3-fluoro-4-methoxy-phenyl)imidazo[1,2-a]pyrazin-8-yl ]amino]benzoyl]piperidine-4-carbonyl]piperazine-2-carboxylic acid;
(2S)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic acid;
(2R)-4-[1-[2-chloro-4-[[3-[4-(cyanomethoxy)-2,3-difluoro-phenyl]imidazo[1,2-a]pyrazin-8-yl]amino]benzoyl]piperidine-4-carbonyl]piperazine-2-carboxylic acid;
(2S)-4-[4-[4-[[3-[4-(difluoromethoxy)phenyl]imidazo[1,2-a]pyrazin-8-yl]amino]-1,2-methyl-benzoyl ]piperazine-1-carbonyl]piperazine-2-carboxylic acid;
1-[4-[1,2-chloro-4-[[3-(2,3-difluoro-4-methoxy-phenyl)imidazo[1,2-a]pyrazin-8-yl ]amino]benzoyl]piperazin-1-yl]-2-(methylamino)ethanone;
2-[3-chloro-4-[8-[3-chloro-4-[4-[3-(dimethylamino)propyl]piperazine-1-carbonyl ]anilino]imidazo[1,2-a]pyrazin-3-yl]-1,2-fluoro-phenoxy]acetonitrile;
[4-(aminomethyl)-1-piperidyl]-[1,2-chloro-4-[[3-(2,3-difluoro-4-prop-1,2-ynoxy-phenyl) imidazo[1,2-a]pyrazin-8-yl]amino]phenyl]methanone;
2-[3-chloro-4-[8-[3-chloro-4-[4-(1,2-pyrrolidin-1-ylethyl)piperazine-1-carbonyl ]anilino]imidazo[1,2-a]pyrazin-3-yl]-1,2-fluoro-phenoxy]acetonitrile;
[2-chloro-4-[[3-(2-chloro-3-fluoro-4-methoxy-phenyl)imidazo[1,2-a]pyrazin-8-yl ]amino]phenyl]-[4-[1,2-(dimethylamino)ethyl]piperazin-1-yl]methanone;
1,2-[4-[8-[3-chloro-4-[4-[1,2-(dimethylamino)ethyl]piperazine-1-carbonyl ]anilino]imidazo[1,2-a]pyrazin-3-yl]-2,3-difluoro-phenoxy]propanenitrile;
[2-chloro-4-[[3-[4-(difluoromethoxy)phenyl]imidazo[1,2-a]pyrazin-8-yl]amino]phenyl]-[4-[2-(dimethylamino)ethyl]piperazin-1-yl]methanone;
2-[4-[8-[3-chloro-4-[4-(1H-imidazol-5-yl)piperidine-1-carbonyl]anilino]imidazo[1,2-a]pyrazin-3-yl]-2,3-difluoro-phenoxy]acetonitrile;
2-[4-[8-[3-chloro-4-[4-(1,2,4-triazol-4-yl)piperidine-1-carbonyl]anilino]imidazo[1,2-a]pyrazin-3-yl]-2,3-difluoro-phenoxy]acetonitrile;
2-[4-[8-[3-chloro-4-[4-(4-methyl-1,2,4-triazol-3-yl)piperidine-1-carbonyl]anilino]imidazo[1,2-a]pyrazin-3-yl]-2,3-difluoro-phenoxy]acetonitrile;
2-[4-[8-[3-chloro-4-(morpholine-4-carbonyl)anilino]imidazo[1,2-a]pyrazin-3-yl]-2,3-difluoro-phenoxy]acetonitrile;
2-[4-[8-[3-chloro-4-[4-[3-(dimethylamino)propyl]piperazine-1-carbonyl]anilino]imidazo[1,2-a]pyrazin-3-yl]-2,3-difluoro-phenoxy]propanenitrile;
[4-(aminomethyl)-1-piperidyl]-[2-chloro-4-[[3-[3-fluoro-4-(4-hydroxybut-2-ynoxy)phenyl]imidazo[1,2-a]pyrazin-8-yl]amino]phenyl]methanone;
3-amino-1-[4-[2-chloro-4-[[3-(3-fluoro-4-methoxy-phenyl)imidazo[1,2-a]pyrazin-8-yl]amino]benzoyl]piperazin-1-yl]-2-hydroxy-propan-1-one;
[2-chloro-4-[[3-(3-fluoro-4-methoxy-phenyl)imidazo[1,2-a]pyrazin-8-yl]amino]phenyl]-[4-[3-(hydroxymethyl)piperazine-1-carbonyl]-1-piperidyl]methanone;
[2-chloro-4-[[3-(3-fluoro-4-methoxy-phenyl)imidazo[1,2-a]pyrazin-8-yl]amino]phenyl]-(4-pyrrolidin-3-ylpiperazin-1-yl)methanone;
[2-bromo-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
1-[2-chloro-4-[[3-[4-(cyanomethoxy)-2,3-difluoro-phenyl]imidazo[1,2-a]pyrazin-8-yl]amino]benzoyl]-N-[2-(methylamino)ethyl]piperidine-4-carboxamide;
2-[3-chloro-4-[8-[3-chloro-4-[4-[2-(hydroxymethyl)piperazine-1-carbonyl]piperidine-1-carbonyl]anilino]imidazo[1,2-a]pyrazin-3-yl]-2-fluoro-phenoxy]acetonitrile;
[4-[2-(azetidin-1-yl)ethyl]piperazin-1-yl]-[2-chloro-4-[[3-(3-fluoro-4-methoxy-phenyl)imidazo[1,2-a]pyrazin-8-yl]amino]phenyl]methanone;
[2-chloro-4-[[3-(3-fluoro-4-methoxy-phenyl)imidazo[1,2-a]pyrazin-8-yl]amino]phenyl]-[4-[2-(cyclopropylamino)ethyl]piperazin-1-yl]methanone;
N-[[1-[2-chloro-4-[[3-[4-(cyanomethoxy)-2,3-difluoro-phenyl]imidazo[1,2-a]pyrazin-8-yl]amino]benzoyl]-4-piperidyl]methyl]pyridine-4-carboxamide;
[2-chloro-4-[[3-(3-fluoro-4-methoxy-phenyl)imidazo[1,2-a]pyrazin-8-yl]amino]phenyl]-[4-(1H-imidazol-5-ylmethyl)piperazin-1-yl]methanone;
[4-(azetidin-3-yl)piperazin-1-yl]-[2-chloro-4-[[3-(3-fluoro-4-methoxy-phenyl)imidazo[1,2-a]pyrazin-8-yl]amino]phenyl]methanone;
2-[4-[8-[3-chloro-4-(4-pyrimidin-2-ylpiperazine-1-carbonyl)anilino]imidazo[1,2-a]pyrazin-3-yl]-2,3-difluoro-phenoxy]acetonitrile;
2-[4-[8-[3-chloro-4-[4-(4-pyridyl)piperidine-1-carbonyl]anilino]imidazo[1,2-a]pyrazin-3-yl]-2,3-difluoro-phenoxy]acetonitrile;
[2-chloro-4-[[3-(3-fluoro-4-methoxy-phenyl)imidazo[1,2-a]pyrazin-8-yl]amino]phenyl]-[4-[2-(dimethylamino)ethyl]-4-hydroxy-1-piperidyl]methanone;
[4-[[3-(2,3-difluoro-4-methoxy-phenyl)imidazo[1,2-a]pyrazin-8-yl]amino]-2,6-difluoro-phenyl]-[4-[(2S,4R)-4-hydroxypyrrolidine-2-carbonyl]piperazin-1-yl]methanone;
[2-chloro-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
2-[4-[8-[4-[4-[2-(azetidin-1-yl)ethyl]piperazine-1-carbonyl]-3-chloro-anilino]imidazo[1,2-a]pyrazin-3-yl]-2,3-difluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxylic acid;
2-[4-[8-[3-chloro-4-[4-[2-(dimethylamino)acetyl]piperazine-1-carbonyl]anilino]imidazo[1,2-a]pyrazin-3-yl]-2,3-difluoro-phenoxy]acetonitrile;
[2-chloro-4-[[3-(2,3-difluoro-4-methoxy-phenyl)imidazo[1,2-a]pyrazin-8-yl]amino]phenyl]-[4-[(2S,4R)-4-hydroxypyrrolidine-2-carbonyl]piperazin-1-yl]methanone;
2-[4-[8-[3-chloro-4-[4-[(3R)-3-(hydroxymethyl)piperazine-1-carbonyl]piperazine-1-carbonyl]anilino]imidazo[1,2-a]pyrazin-3-yl]-2,3-difluoro-phenoxy]acetonitrile;
2-[4-[8-[3-chloro-4-[4-[(3S)-3-(hydroxymethyl)piperazine-1-carbonyl]piperazine-1-carbonyl]anilino]imidazo[1,2-a]pyrazin-3-yl]-2,3-difluoro-phenoxy]acetonitrile;
2-[4-[8-[3-chloro-4-[4-[(2S,4R)-4-hydroxypyrrolidine-2-carbonyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-3-carboxamide;
2-[4-[8-[3-chloro-4-[4-[2-[3-(hydroxymethyl)pyrrolidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[(3R)-3-(hydroxymethyl)piperazine-1-carbonyl]piperidine-1-carbonyl]anilino]imidazo[1,2-a]pyrazin-3-yl]-2,3-difluoro-phenoxy]acetonitrile;
2-[4-[8-[3-chloro-4-[4-[2-(methylamino)ethyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(3R)-3-(hydroxymethyl)piperazine-1-carbonyl]piperidine-1-carbonyl]anilino]imidazo[1,2-a]pyrazin-3-yl]-2-fluoro-phenoxy]acetonitrile;
2-[4-[8-[3-chloro-4-[4-[2-[3-(hydroxymethyl)azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[(3S)-3-(hydroxymethyl)piperazine-1-carbonyl]piperidine-1-carbonyl]anilino]imidazo[1,2-a]pyrazin-3-yl]-2,3-difluoro-phenoxy]acetonitrile;
2-[4-[8-[3-chloro-4-[4-[2-[2-(hydroxymethyl)pyrrolidin-1-yl]ethyl]piperazine-1-carbonyl]anilino]imidazo[1,2-a]pyrazin-3-yl]-2,3-difluoro-phenoxy]acetonitrile;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]azetidine-3-carboxamide;
2-[4-[8-[3-chloro-4-[4-[2-(3-hydroxypyrrolidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(3-hydroxyazetidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(methylamino)acetyl]piperazine-1-carbonyl]anilino]imidazo[1,2-a]pyrazin-3-yl]-2,3-difluoro-phenoxy]acetonitrile;
2-[3-chloro-4-[8-[3-chloro-4-[4-[(3S)-3-(hydroxymethyl)piperazine-1-carbonyl]piperidine-1-carbonyl]anilino]imidazo[1,2-a]pyrazin-3-yl]-2-fluoro-phenoxy]acetonitrile;
2-[4-[8-[3-chloro-4-[4-[2-(3-methoxyazetidin-1-yl)ethyl]piperazine-1-carbonyl]anilino]imidazo[1,2-a]pyrazin-3-yl]-2,3-difluoro-phenoxy]acetonitrile;
N-(3-amino-2-hydroxy-propyl)-1-[2-chloro-4-[[3-(3-fluoro-4-methoxy-phenyl)imidazo[1,2-a]pyrazin-8-yl]amino]benzoyl]piperidine-4-carboxamide;
2-[4-[8-[3-chloro-4-[4-[2-(3-methylsulfonylazetidin-1-yl)ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-[(dimethylamino)methyl]azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-(dimethylamino)pyrrolidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[4-(dimethylamino)-1-piperidyl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-[3-(dimethylamino)azetidin-1-yl]ethyl]piperazine-1-carbonyl]anilino]imidazo[1,2-a]pyrazin-3-yl]-2,3-difluoro-phenoxy]acetonitrile;
2-[4-[8-[3-chloro-4-[4-[2-(3-methylsulfonylpyrrolidin-1-yl)ethyl]piperazine-1-carbonyl]anilino]imidazo[1,2-a]pyrazin-3-yl]-2,3-difluoro-phenoxy]acetonitrile;
[4-[[3-[4-(difluoromethoxy)-2,3-difluoro-phenyl]imidazo[1,2-a]pyrazin-8-yl]amino]-2-fluoro-phenyl]-[4-[1-(pyrrolidin-3-ylmethyl)piperidine-4-carbonyl]piperazin-1-yl]methanone;
[2-chloro-4-[[3-[4-(difluoromethoxy)-2,3-difluoro-phenyl]imidazo[1,2-a]pyrazin-8-yl]amino]phenyl]-[4-[(2S,4R)-4-hydroxypyrrolidine-2-carbonyl]piperazin-1-yl]methanone;
[2-chloro-4-[[3-[2,3-difluoro-4-(fluoromethoxy)phenyl]imidazo[1,2-a]pyrazin-8-yl]amino]phenyl]-[4-[(2S,4R)-4-hydroxypyrrolidine-2-carbonyl]piperazin-1-yl]methanone;
[2-chloro-4-[[3-(2,3-difluoro-4-methoxy-phenyl)imidazo[1,2-a]pyrazin-8-yl]amino]phenyl]-[4-hydroxy-4-(methylaminomethyl)-1-piperidyl]methanone;
[2-chloro-4-[[3-(3-fluoro-4-methoxy-phenyl)imidazo[1,2-a]pyrazin-8-yl]amino]phenyl]-[4-hydroxy-4-(methylaminomethyl)-1-piperidyl]methanone;
[2-bromo-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
[2-bromo-4-[[3-(2,3-difluoro-4-methoxy-phenyl)imidazo[1,2-a]pyrazin-8-yl]amino]-6-fluoro-phenyl]-[4-[(2S,4R)-4-hydroxypyrrolidine-2-carbonyl]piperazin-1-yl]methanone;
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]-2-oxo-ethyl]pyrrolidine-3-carboxylic acid;
1-[2-chloro-4-[[3-(3-fluoro-4-methoxy-phenyl)imidazo[1,2-a]pyrazin-8-yl]amino]benzoyl]-N-[(2S,3R,4R,5R)-2,3,4,5,6-pentahydroxyhexyl]piperidine-4-carboxamide;
2-[1-[2-chloro-4-[[3-(3-fluoro-4-methoxy-phenyl)imidazo[1,2-a]pyrazin-8-yl]amino]benzoyl]-4-hydroxy-4-piperidyl]-N,N-dimethyl-acetamide;
2-[4-[8-[3-chloro-4-[4-[2-[3-(1H-tetrazol-5-yl)azetidin-1-yl]ethyl]piperazine-1-carbonyl]anilino]imidazo[1,2-a]pyrazin-3-yl]-2,3-difluoro-phenoxy]acetonitrile; and
1-[2-[4-[2-chloro-4-[[3-[4-(cyanomethoxy)-2,3-difluoro-phenyl]imidazo[1,2-a]pyrazin-8-yl]amino]benzoyl]piperazin-1-yl]ethyl]pyrrolidine-2-carboxylic acid.

29. The compound of formula (I) according to claim 1, or a pharmaceutically acceptable salt thereof, wherein the compound of formula (I) is selected from:
[2-chloro-4-[[3-(3-fluoro-4-methoxy-phenyl)imidazo[1,2-a]pyrazin-8-yl]amino]phenyl]-[4-[2-(dimethylamino)ethyl]piperazin-1-yl]methanone;
1-[2-chloro-4-[[3-(3-fluoro-4-methoxy-phenyl)imidazo[1,2-a]pyrazin-8-yl]amino]benzoyl]-N-[2-(methylamino)ethyl]piperidine-4-carboxamide;
1-[4-[2-chloro-4-[[3-(3-fluoro-4-methoxy-phenyl)imidazo[1,2-a]pyrazin-8-yl]amino]benzoyl]piperazin-1-yl]-2-(methylamino)ethanone;
2-[5-chloro-4-[8-[3-chloro-4-[4-[3-(hydroxymethyl)piperazine-1-carbonyl]piperidine-1-carbonyl]anilino]imidazo[1,2-a]pyrazin-3-yl]-2-fluoro-phenoxy]acetonitrile;
[2-chloro-4-[[3-(3-fluoro-4-methoxy-phenyl)imidazo[1,2-a]pyrazin-8-yl]amino]phenyl]-[4-[2-(methylamino)ethyl]piperazin-1-yl]methanone;
N-(azetidin-3-ylmethyl)-1-[2-chloro-4-[[3-(3-fluoro-4-methoxy-phenyl)imidazo[1,2-a]pyrazin-8-yl]amino]benzoyl]piperidine-4-carboxamide;
[2-chloro-4-[[3-(3-fluoro-4-methoxy-phenyl)imidazo[1,2-a]pyrazin-8-yl]amino]phenyl]-[4-[2-(dimethylamino)ethyl]-4-hydroxy-1-piperidyl]methanone;
[2-chloro-4-[[3-(2,3-difluoro-4-methoxy-phenyl)imidazo[1,2-a]pyrazin-8-yl]amino]phenyl]-[4-[(2S,4R)-4-hydroxypyrrolidine-2-carbonyl]piperazin-1-yl]methanone;
2-[4-[8-[3-chloro-4-[4-[(2S,4R)-4-hydroxypyrrolidine-2-carbonyl]piperazine-1-carbonyl]anilino]imidazo[1,2-a]pyrazin-3-yl]-2,3-difluoro-phenoxy]acetonitrile; and
2-[3-chloro-4-[8-[3-chloro-4-[4-[(2S,4R)-4-hydroxypyrrolidine-2-carbonyl]piperazine-1-carbonyl]anilino]imidazo[1,2-a]pyrazin-3-yl]-2-fluoro-phenoxy]acetonitrile.

30. A process of manufacturing the compounds of formula (I) according to claim 1, the process comprising:
(i) reacting a carboxylic acid IVa, wherein R³ to R¹¹ are as defined in claim 1, 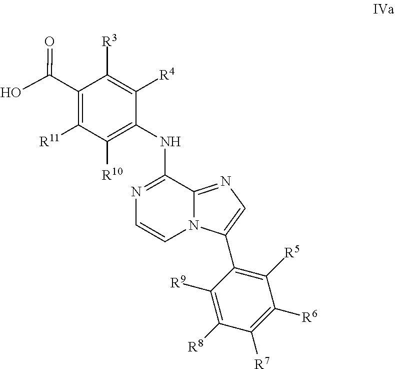
with an amine V, wherein A, R¹ and R² are as defined in claim 1, 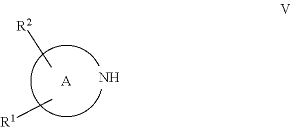
in the presence of a coupling reagent (such as HATU, TBTU, and the like) and a base (such as DIPEA, NEt₃, and the like), to form said compound of formula (I); or
(ii) reacting a compound VI, wherein R¹ to R⁴, R¹⁰, R¹¹ and A are as defined in claim 1 and X is halogen, 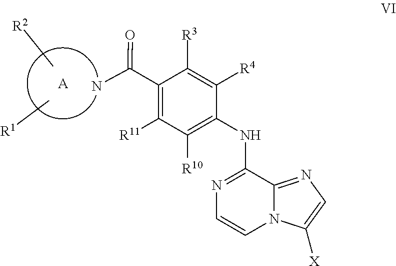
with a boronic acid VII, wherein R⁵ to R⁹ are as defined in claim 1 and Y is a boronic acid or a boronic acid ester, 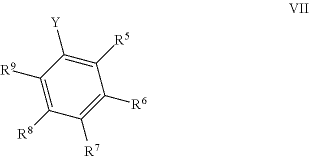
in the presence of a transition metal catalyst (such as PdCl₂(dppf)-CH₂Cl₂ adduct, Pd(PPh₃)₄, and the like) and a base (such as K₃PO₄, NaOtBu, and the like),
to form said compound of formula (I).

31.-32. (canceled)

33. A pharmaceutical composition comprising a compound of formula (I) according to claim 1, or a pharmaceutically acceptable salt thereof, and a therapeutically inert carrier.

34.-39. (canceled)

40. A method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, the method comprising administering a compound of formula (I) according to claim 1, or a pharmaceutically acceptable salt thereof, to a mammal in need thereof.

41.-44. (canceled)
